(19) United States
(12) Patent Application Publication
(10) Pub. No.: US 2025/0049959 A1
(43) Pub. Date: Feb. 13, 2025

(54) OPTIMIZED EXPRESSION CASSETTES FOR GENE THERAPY

(71) Applicant: Tenaya Therapeutics, Inc., South San Francisco, CA (US)

(72) Inventors: Huanyu ZHOU, San Mateo, CA (US); Christopher A. REID, Walnut Creek, CA (US)

(21) Appl. No.: 18/919,698

(22) Filed: Oct. 18, 2024

Related U.S. Application Data
(62) Division of application No. 18/468,594, filed on Sep. 15, 2023, now Pat. No. 12,151,001.
Application No. 18/468,594 is a continuation of application No. PCT/US2022/073574, filed on Jul. 8, 2022.
(60) Provisional application No. 63/219,651, filed on Jul. 8, 2021.

(51) Int. Cl. A61K 48/00; C07K 14/47; C07K 14/705; C12N 15/86

(52) U.S. Cl. CPC A61K 48/0058; C07K 14/47; C07K 14/4702; C07K 14/705; C12N 15/86; C12N 2750/14143; C12N 2750/14171; C12N 2830/008; C12N 2830/42; C12N 2830/48; C12N 2830/50

(57) ABSTRACT

In some aspects, cardiac-specific expression cassettes are provided herein. In some aspects, provided herein is an expression cassette comprising a polynucleotide sequence encoding a gene product for therapy of a heart disease, wherein the polynucleotide sequence is operably linked to promoter (e.g., a cardiac-specific promoter), and optionally an enhancer (e.g., a cardiac-specific enhancer). In some aspects, the disclosure provides recombinant adeno-associated virus (rAAV) virions, comprising a capsid protein and a viral genome comprising an expression cassette comprising a polynucleotide sequence encoding a therapeutic gene product, e.g., dwarf open reading frame (DWORF) polypeptide, operably linked to a promoter, the expression cassette flanked by inverted terminal repeats. The disclosure further provides pharmaceutical compositions and methods of treating or preventing heart disease.

### CROSS-REFERENCE TO RELATED APPLICATION

This application is a divisional of U.S. application Ser. No. 18/468,594, filed on Sep. 15, 2023, which is a continuation of International Patent Application No. PCT/US2022/073574, filed on Jul. 8, 2022, which claims the benefit of U.S. Provisional Patent Application No. 63/219,651, filed on Jul. 8, 2021, the contents of each of which are incorporated by reference herein in their entireties.

### FIELD OF THE INVENTION

This invention relates generally to gene therapies, e.g., optimized gene expression cassettes, recombinant adeno-associated virus (AAV) virions, and methods for treating and preventing heart disease using the same.

### REFERENCE TO AN ELECTRONIC SEQUENCE LISTING

The contents of the electronic sequence listing (TENA_021_04US_SeqList_ST26.xml; Size: 430,378 bytes; and Date of Creation: Oct. 16, 2024) are herein incorporated by reference in their entirety.

### BACKGROUND

Cardiomyopathy is responsible for about half of cardiac-related deaths. It is estimated that about 1 in 250 to 1 in 10,000 adults are affected by some form of cardiomyopathy (McKenna et al. Circ Res. 121:722-730 (2017)). Despite major efforts in screening, diagnostics, and therapeutic strategies, the prevalence of cardiomyopathies and incidence of cardiomyopathy-related deaths remains high (Brieler et al. Am Fam Physician. 96:640-646 (2017)).

Cardiomyopathy refers to a collection of conditions of the heart that occur when its ability to pump blood is reduced. Reduction in proper functioning, such as a contractile dysfunction, of the heart muscle can lead to myocardial infarction, heart failure, blood clots, valve problems, and cardiac arrest. Cardiomyopathies can be separated into primary and secondary categories that result in varied phenotypes (McKenna et al. Circ Res. 121:722-730 (2017)). Primary cardiomyopathies can be genetic, acquired, or mixed in etiology. Genetic cardiomyopathies are inherited and include arrhythmogenic right ventricular dysplasia, hypertrophic, ion channel disorders, left ventricular compaction, and mitochondrial myopathies. Acquired cardiomyopathies are due primarily to non-secondary, non-genetic causes that lead to cardiac complications and include myocarditis, peripartum, tachycardia-induced cardiomyopathy, and stress-induced cardiomyopathy. Cardiomyopathies with mixed etiology are caused by a combination of non-genetic and genetic factors, and include dilated cardiomyopathy and restrictive cardiomyopathy. Secondary cardiomyopathies refer to heart disease resulting from an extra cardiovascular cause. The underlying causes of secondary cardiomyopathies can be endocrine, infection, exposure to toxins, autoimmune related, nutritional, and/or neuromuscular.

Cardiomyocytes play a central role cardiomyopathy. Cardiomyocytes, also called cardiac muscle cells, cardiac myocytes, or myocardiocytes, are cardiac cells that make up the heart muscle and are responsible for the contractile function that allows the heart to act as a pump. There are many mechanisms that reduce cardiomyocytes' ability to function properly (Dadson et al. Clin Sci (Lond) 131:1375-1392 (2017)). In arrhythmogenic right ventricular cardiomyopathy, progressive replacement of cardiomyocytes with fibrotic tissue results in the electrical isolation of cardiomyocytes and atrophy of the ventricular myocardium, the major structure responsible for contractile function in the heart. In mitochondrial cardiomyopathy, a deficiency in ATP production has a direct effect on contractile function in cardiomyocytes that have a high metabolic demand. Cardiomyopathies also emerge as a result of abnormal contractile function resulting from loss of normal Ca², ion-release, uptake, and sequestration processes due to loss of activity in regulatory enzymes, such as sarco/endoplasmic reticulum calcium ATPase (SERCA) (Lennon et al. Int JMol Med. 7:131-41 (2001)).

Treatment strategies for cardiomyopathy are needed.

Gene therapy approaches for the treatment of heart disease often employ vectors configured to transduce cardiac cells and to express a transgene in a cardiac tissue-specific manner. Adeno-associated virus (AAV) vectors, cardiac-specific promoters, or both in combination, may be used to deliver a polynucleotide encoding a gene product (e.g., a therapeutic protein) to heart tissue and thereby express the gene product in that tissue to treat the heart disease.

However, achieving high expression of gene products remains challenging, especially in cardiac cells.

Given these challenges, there remains a need in the art for improved gene therapy vectors, especially for heart disease.

### SUMMARY

In some aspects, the present invention relates generally to vectors for delivery of a polynucleotide encoding a dwarf open reading frame (DWORF) or another transgene to cardiac cells, e.g., cardiomyocytes. Disclosed herein are recombinant adeno-associated virus virions (rAAV virions), including expression cassettes and capsid proteins, that effectively deliver DWORF polynucleotides into cardiac cells, along with related compositions and methods. In any aspects described herein where DWORF transgene is referenced, DWORF can be substituted by a reference to another transgene expression of which in cardiac cells is desired. In some embodiments, where AAV-based expression vectors and virions are referenced, the disclosure also contemplates use of other viral and non-viral vectors for delivery of transgenes. In particular, any viral and non-viral vectors that can be used for delivery of transgenes into cardiac cells are provided herein.

In some aspects, provided herein is an expression cassette comprising a polynucleotide sequence comprising:
i) one or more promoters, optionally wherein the one or more promoters are cardiac-specific promoters; and
ii) one or more copies of a transgene, optionally wherein the transgene encodes a polypeptide for treating or preventing a heart disease or alleviating symptoms associated with a heart disease; wherein in addition to elements (i) and (ii), the expression cassette comprises one or more of the following:
iii) one or more enhancers, optionally wherein the one or more enhancers are cardiac-specific enhancers;
iv) the one or more copies of a transgene is at least two copies of the transgene;
v) the polynucleotide sequence comprises one or more introns; and/or
vi) at least one copy of the one or more copies of the transgene is codon-optimized.

In some embodiments of the expression cassette described above, in addition to elements (i) and (ii), the expression cassette comprises one, two, three or all four of elements (iii), (iv), (v) and (vi) (any combination of elements (iii), (iv), (v) and (vi) can be used). In some embodiments of the expression cassette described above, in addition to elements (i) and (ii), the expression cassette comprises one or more enhancers, wherein the one or more enhancers are cardiac-specific enhancers, and/or the polynucleotide sequence comprises one or more introns. In some embodiments of the expression cassette described above, in addition to elements (i) and (ii), the expression cassette comprises one or more enhancers, wherein the one or more enhancers are cardiac-specific enhancers, and the polynucleotide sequence comprises one or more introns. In some embodiments, the one or more introns improve, or can improve, the efficiency of transgene expression. In some embodiments of the expression cassette described above, in addition to elements (i) and (ii), the expression cassette comprises two copies of the transgene, wherein the two copies are not identical, optionally wherein first copy is codon-optimized and second copy is not codon-optimized nucleotide sequence encoding the transgene. In some embodiments of the expression cassette described above, in addition to elements (i) and (ii), the expression cassette comprises two copies of the transgene, wherein the two copies are not identical to each other, optionally wherein first copy is codon-optimized and second copy is not codon-optimized nucleotide sequence encoding the transgene, and further the polynucleotide sequence comprises one or more introns. In some embodiments of the expression cassette described above, in addition to elements (i) and (ii), the expression cassette comprises two copies of the transgene, wherein the two copies are not identical to each other, optionally wherein first copy is codon-optimized and second copy is not codon-optimized nucleotide sequence encoding the transgene, and further the polynucleotide sequence comprises one or more introns, and further the polynucleotide sequence comprises one or more enhancers (e.g., wherein the one or more enhancers are cardiac-specific enhancers). In some embodiments, the one or more introns improve, or can improve, the efficiency of transgene expression. In some embodiments where two copies of the transgene are used, two copies of the promoters are also used.

In some embodiments of the expression cassette, the polynucleotide sequence comprises one or more promoters, wherein the one or more promoters are cardiac-specific enhancers. In some embodiments of the expression cassette, at least one promoter is a cardiac-specific promoter, or all of the promoters are cardiac-specific promoters. In some embodiments of the expression cassette, the polynucleotide sequence comprises a single promoter. In some embodiments of the expression cassette, the polynucleotide sequence comprises two promoters. In some embodiments of the expression cassette, at least one promoter of the one or more promoters is a chicken cTnT promoter. In some embodiments, the chicken cTnT promoter shares at least 90%, 95%, 96%, 97%, 98%, or 99% identity to SEQ ID NO: 11. In some embodiments, the chicken cTnT promoter comprises SEQ ID NO: 11. In some embodiments of the expression cassette, at least one promoter of the one or more promoters is a human cTnT promoter. In some embodiments, the human cTnT promoter shares at least 90%, 95%, 96%, 97%, 98%, or 99% identity to SEQ ID NO: 12 or SEQ ID NO: 13. In some embodiments, the human cTnT promoter comprises SEQ ID NO: 12 or SEQ ID NO: 13. In some embodiments, the expression cassette comprises a chicken cTnT promoter and a human cTnT promoter.

In some embodiments of the expression cassette, the polynucleotide sequence comprises one or more copies of a transgene, wherein the transgene encodes a polypeptide for treating or preventing a heart disease or alleviating symptoms associated with a heart disease.

In some embodiments of the expression cassette, one or more copies of a transgene is at least two copies of the transgene. In some embodiments of the expression cassette, one or more copies of a transgene is two copies of the transgene. In some embodiments, one or more copies of a transgene is at least two copies of the transgene, and wherein the polynucleotide sequence comprises at least two promoters each operably linked to the at least two copies of the transgene. In some embodiments, one or more copies of a transgene is two copies of the transgene, and wherein the polynucleotide sequence comprises two promoters each operably linked to the two copies of the transgene. In some embodiments of the expression cassette comprising two copies of the transgene, the two “copies” are not identical. While not being bound by any theory, using two nucleic acid sequences encoding a polypeptide that are not identical may prevent DNA recombination within the vector. In some embodiments, the expression cassette comprises one copy of the transgene that has the original DNA sequence encoding a polypeptide and one copy of the transgene that has a codon optimized DNA sequence encoding the polypeptide. In some embodiments of the expression cassette comprising two copies of the transgene, the first copy of the transgene is sufficiently different from the second copy of the transgene to prevent DNA recombination.

In some embodiments of the expression cassette, the polynucleotide sequence comprises one or more enhancers, optionally wherein the one or more enhancers are cardiac-specific enhancers. In some embodiments of the expression cassette, the polynucleotide sequence comprises two or more enhancers (e.g., 2, 3, or 4 enhancers). In some embodiments of the expression cassette, one or more enhancers are cardiac-specific enhancers (e.g., at least one enhancer is a cardiac-specific enhancer, or 2, 3, or 4, or all of the enhancers are cardiac-specific enhancers). In some embodiments of the expression cassette, the polynucleotide sequence comprises one enhancer. In some embodiments of the expression cassette, the polynucleotide sequence comprises no enhancers. In some embodiments, the one or more cardiac-specific enhancers are selected from a ACTC1 enhancer and a αMHC enhancer. In some embodiments, the ACTC1 enhancer shares at least 90%, 95%, 96%, 97%, 98%, or 99% identity to SEQ ID NO: 78. In some embodiments, the ACTC1 enhancer comprises SEQ ID NO: 78. In some embodiments, the αMHC enhancer shares at least 90%, 95%, 96%, 97%, 98%, or 99% identity to SEQ ID NO: 79. In some embodiments, the αMHC enhancer comprises SEQ ID NO: 79. In some embodiments, the expression cassette comprises an αMHC enhancer and an ACTC1 enhancer. In some embodiments of the expression cassette, the enhancer sequence comprises an αMHC enhancer followed by an ACTC1 enhancer. In some embodiments of the expression cassette, the enhancer sequence comprises an ACTC1 enhancer followed by an αMHC enhancer.

In some embodiments of the expression cassette, the polynucleotide sequence comprises one or more introns. In some embodiments of the expression cassette, the polynucleotide sequence comprises one intron. In some embodiments of the expression cassette, the polynucleotide sequence comprises two introns. In some embodiments of the expression cassette, the polynucleotide sequence comprises more than two introns. In some embodiments of the expression cassette, one or more introns are the same. In some embodiments of the expression cassette, one or more introns are different from each other. In some embodiments, the expression cassette comprises an intron and the intron is selected from a CMV intron and a chimeric intron. In some embodiments, the CMV intron shares at least 90%, 95%, 96%, 97%, 98%, or 99% identity to SEQ ID NO: 80. In some embodiments, the CMV intron comprises SEQ ID NO: 80. In some embodiments, the chimeric intron shares at least 90%, 95%, 96%, 97%, 98%, or 99% identity to SEQ ID NO: 81. In some embodiments, the chimeric intron comprises SEQ ID NO: 81. In some embodiments, the expression cassette comprises a CMV intron and a chimeric intron. In some embodiments, the expression cassette does not comprise an intron (e.g., does not comprise a CMV intron or a chimeric intron).

In some embodiments of the expression cassette, at least one copy of the one or more copies of the transgene is codon-optimized (e.g., codon-optimized for optimum human expression). In some embodiments of the expression cassette, two copies of the transgene are codon-optimized. In some embodiments of the expression cassette, first copy of the transgene is codon-optimized and second copy of the transgene is not codon optimized (e.g., original DNA sequence) or is otherwise different from the first copy. In some embodiments of the expression cassette, the first copy of the transgene is sufficiently different from the second copy of the transgene to prevent DNA recombination.

In some embodiments, the expression cassette further comprises one or more (e.g., two) post-transcriptional regulatory elements (“PTRE”). In some embodiments, the expression cassette further comprises one or more (e.g., two) WPRE sequences. In some embodiments, the expression cassette comprises one WPRE sequence. In some embodiments, the WPRE sequence shares at least 90%, 95%, 96%, 97%, 98%, or 99% identity to SEQ ID NO: 26. In some embodiments, the WPRE sequence comprises SEQ ID NO: 26. In some embodiments, the expression cassette does not comprise a WPRE sequence.

In some embodiments, the expression cassette further comprises one or more polyadenylation sequences (“p(A)”). In some embodiments, the expression cassette comprises one polyadenylation sequence. In some embodiments, the expression cassette comprises two polyadenylation sequences. In some embodiments, the polyadenylation sequence is selected from a BGH polyadenylation sequence and a SV40 polyadenylation sequence. In some embodiments, the BGH polyadenylation sequence shares at least 90%, 95%, 96%, 97%, 98%, or 99% identity to SEQ ID NO: 27. In some embodiments, the BGH polyadenylation sequence comprises SEQ ID NO: 27. In some embodiments, the SV40 polyadenylation sequence shares at least 90%, 95%, 96%, 97%, 98%, or 99% identity to SEQ ID NO: 28. In some embodiments, the SV40 polyadenylation sequence comprises SEQ ID NO: 28. In some embodiments, the expression cassette comprises a BGH polyadenylation sequence and a SV40 polyadenylation sequence.

In some embodiments, the expression cassette comprises 5′ to 3′ arrangement of elements selected from any one of the following:
(i) 5′-promoter-intron-transgene-PTRE-p(A)-3′;
(ii) 5′-promoter-transgene-PTRE-p(A)-promoter-transgene-PTRE-p(A);
(iii) 5′-enhancer-promoter-transgene-PTRE-p(A)-3′;
(iv) 5′-enhancer-promoter-intron-transgene-PTRE-p(A)-3′;
(v) 5′-enhancer-enhancer-promoter-transgene-PTRE-p(A)-3′;
(vi) 5′-enhancer-enhancer-promoter-intron-transgene-PTRE-p(A)-3′;
(vii) 5′-enhancer-promoter-intron-transgene-PTRE-p(A)-p(A)-transgene-intron-promoter-enhancer-3′;
(viii) 5′-enhancer-promoter-intron-transgene-PTRE-p(A)-enhancer-promoter-intron-transgene-p(A)-3′;
(ix) 5′-p(A)-PTRE-transgene-intron-promoter-enhancer-enhancer-promoter-intron-transgene-p(A)-3′;
(x) 5′-promoter-intron-transgene-PTRE-p(A)-p(A)-transgene-intron-promoter-3′;
(xi) 5′-promoter-intron-transgene-PTRE-p(A)-promoter-intron-transgene-p(A)-3′; and
(xii) 5′-p(A)-PTRE-transgene-intron-promoter-promoter-intron-transgene-p(A)-3′.

In some embodiments of the expression cassette, the transgene has an increased expression level compared to an expression cassette comprising a polynucleotide having an arrangement of elements from 5′ to 3′ comprising: 5′-promoter-transgene-WPRE-p(A)-3′. In some embodiments, the increased expression level is between about 1.5-fold and about 150-fold. In some embodiments, the increased expression level is at least 2 fold, at least 5 fold, at least 10 fold, at least 25 fold, at least 50 fold, at least 75 fold, or at least 100 fold.

In some embodiments, the expression cassette is flanked by ITRs. In some embodiments, the ITRs share at least 90%, 95%, 96%, 97%, 98%, or 99% identity to one or more of SEQ ID NO: 14 and SEQ ID NO: 15. In some embodiments, the ITRs comprise one or more of SEQ ID NO: 14 and SEQ ID NO: 15.

In some embodiments, the expression cassette comprises from about 1.9 kb to about 3.7 kb. In some embodiments, the expression cassette comprises from about 2.5 kb to about 3.7 kb, optionally from about 2.8 kb to about 3.6 kb.

In some embodiments, the transgene in the expression cassette encodes a polypeptide useful in the treatment of a heart disease or disorder, optionally when a wild type copy of the gene is introduced to a subject. In some embodiments, the transgene in the expression cassette encodes a polypeptide which is associated with a heart disease (e.g., where loss of function mutations in the gene encoding the polypeptide are associated with heart disease).

In some embodiments, the transgene in the expression cassette encodes a polypeptide selected from: DWORF, JPH2, BAG3, CRYAB, Lamin A isoform of LMNA, Lamin C isoform of LMNA, TNNI3, PLN, LAMP2a, LAMP2b, LAMP2c, DPI isoform of DSP, DPII isoform of DSP, DSG2, and JUP. In some embodiments, the expression cassette comprises a transgene which shares at least 75%, 80%, 90%, 95%, 96%, 97%, 98%, 99% or 100% sequence identity to SEQ ID NO:201, SEQ ID NO:203, SEQ ID NO:205, SEQ ID NO:207, SEQ ID NO:209, SEQ ID NO:211, SEQ ID NO:213, SEQ ID NO:215, SEQ ID NO:217, SEQ ID NO:219, SEQ ID NO:221, SEQ ID NO:223, SEQ ID NO:225, SEQ ID NO:227, or SEQ ID NO:229. In some embodiments, the polypeptide shares at least 75%, 80%, 90%, 95%, 96%, 97%, 98%, 99% or 100% sequence identity to SEQ ID NO:202, SEQ ID NO:204, SEQ ID NO:206, SEQ ID NO:208, SEQ ID NO:210, SEQ ID NO:212, SEQ ID NO:214, SEQ ID NO:216, SEQ ID NO:218, SEQ ID NO:220, SEQ ID NO:222, SEQ ID NO:224, SEQ ID NO:226, SEQ ID NO:228, or SEQ ID NO:230.

In some embodiments, the transgene in the expression cassette encodes a DWORF polypeptide. In some embodiments, the transgene shares at least 75%, 80%, 90%, 95%, 96%, 97%, 98%, 99% or 100% sequence identity to SEQ ID NO:2, SEQ ID NO:4, SEQ ID NO:6, SEQ ID NO:8, SEQ ID NO:10, SEQ ID NO:33, SEQ ID NO:44, SEQ ID NO:76, or SEQ ID NO:77. In some embodiments, the polypeptide shares at least 75%, 80%, 90%, 95%, 96%, 97%, 98%, 99% or 100% sequence identity to SEQ ID NO:1, SEQ ID NO:3, SEQ ID NO:5, SEQ ID NO:7, SEQ ID NO:9, SEQ ID NO:32, or SEQ ID NO:43.

In some embodiments, the expression cassette comprises a 5′ to 3′ arrangement of elements selected from any one of the following:
(i) 5′-human or chicken TnT promoter-chimeric intron-transgene-WPRE-p(A)-3′;
(ii) 5′-first human or chicken TnT promoter-first copy of transgene-WPRE-p(A)—second human or chicken TnT promoter—second copy of transgene-WPRE-p(A), optionally where the first and second promoter sequences and the first and second copies of the transgene are in the same forward orientation;
(iii) 5′-human TnT promoter-CMV intron-transgene-WPRE-bGHpA-3′;
(iv) 5′-ACTC1e enhancer-cardiac-human TnT promoter-transgene-WPRE-bGHpA-3′;
(v) 5′-αMHCe enhancer-human TnT promoter-transgene-WPRE-bGHpA-3′;
(vi) 5′-ACTC1e enhancer-human TnT promoter-CMV intron-transgene-WPRE-bGHpA-3′;
(vii) 5′-αMHCe enhancer-human TnT promoter-CMV intron-transgene-WPRE-bGHpA-3′;
(viii) 5′-ACTC1e enhancer-αMHCe enhancer-human TnT promoter-transgene-WPRE-bGHpA-3′;
(ix) 5′-αMHCe enhancer-ACTC1e enhancer-human TnT promoter-transgene-WPRE-bGHpA-3′;
(x) 5′-ACTC1e enhancer-αMHCe enhancer-human TnT promoter-CMV intron-transgene-WPRE-bGHpA-3′;
(xi) 5′-αMHCe enhancer-ACTC1e enhancer-human TnT promoter-CMV intron-transgene-WPRE-bGHpA-3′;
(xii) 5′-human TnT promoter-transgene with a codon-optimized polynucleotide sequence-WPRE-bGHpA-3′;
(xiii) 5′-αMHCe enhancer-human TnT promoter-CMV intron-first transgene-WPRE-bGHpA-SV40 pA-second transgene (e.g., with a codon-optimized polynucleotide sequence)-chimeric intron-chicken TnT promoter-ACTC1e enhancer-3′, optionally wherein the first transgene and the human TnT promoter are in a forward orientation, and the second transgene and the chicken TnT promoter are in a reverse orientation;
(xiv) 5′-αMHCe enhancer-human TnT promoter-CMV intron-first transgene-WPRE-bGHpA-ACTC1e enhancer-chicken TnT promoter-chimeric intron-second transgene (e.g., with a codon-optimized polynucleotide sequence)-SV40 pA-3′, optionally wherein the first transgene, the human TnT promoter, the second transgene and the chicken TnT promoter are in a forward orientation;
(xv) 5′-bGHpA-WPRE-first transgene-CMV intron-human TnT promoter-αMHCe enhancer-ACTC1e enhancer-chicken TnT promoter-chimeric intron-second transgene (e.g., with a codon-optimized polynucleotide sequence)-SV40 pA-3′, optionally wherein the first transgene and the human TnT promoter are in a reverse orientation, and the second transgene and the chicken TnT promoter are in a forward orientation;
(xvi) 5′-human TnT promoter-CMV intron-first transgene-WPRE-bGHpA-pSV40 pA-second transgene (e.g., with a codon-optimized polynucleotide sequence)-chimeric intron-chicken TnT promoter-3′, optionally wherein the first transgene and the human TnT promoter are in a forward orientation, and the second transgene and the chicken TnT promoter are in a reverse orientation; and
(xvii) 5′-huma TnT promoter-CMV intron-first transgene-WPRE-bGHpA-chicken TnT promoter-chimeric intron-second transgene (e.g., with a codon-optimized polynucleotide sequence)-SV40 pA-3′, optionally wherein the first transgene, the human TnT promoter, the second transgene and the chicken TnT promoter are in a forward orientation; and (xix) 5′-bGHpA-WPRE-first transgene-CMV intron-human TnT promoter-chicken TnT promoter-chimeric intron-second transgene (e.g., with a codon-optimized polynucleotide sequence)-SV40 pA-3′, optionally wherein the first transgene and the human TnT promoter are in a reverse orientation, and the second transgene and the chicken TnT promoter are in a forward orientation.

In some embodiments, the expression cassette comprises 5′ to 3′ arrangement of elements selected from any one of the following:
(i) 5′-ACTC1e enhancer-αMHCe enhancer-human TnT promoter-CMV intron-transgene-WPRE-bGHpA-3′;
(ii) 5′-αMHCe enhancer-ACTC1e enhancer-human TnT promoter-CMV intron-transgene-WPRE-bGHpA-3′;
(iii) 5′-αMHCe enhancer-human TnT promoter-CMV intron-first transgene-WPRE-bGHpA-SV40 pA-second transgene (e.g., with a codon-optimized polynucleotide sequence)-chimeric intron-chicken TnT promoter-ACTC1e enhancer-3′, optionally wherein the first transgene and the human TnT promoter are in a forward orientation, and the second transgene and the chicken TnT promoter are in a reverse orientation;
(iv) 5′-αMHCe enhancer-human TnT promoter-CMV intron-first transgene-WPRE-bGHpA-ACTC1e enhancer-chicken TnT promoter-chimeric intron-second transgene (e.g., with a codon-optimized polynucleotide sequence)-SV40 pA-3′, optionally wherein the first transgene, the human TnT promoter, the second transgene and the chicken TnT promoter are in a forward orientation;
(v) 5′-bGHpA-WPRE-first transgene-CMV intron-human TnT promoter-αMHCe enhancer-ACTC1e enhancer-chicken TnT promoter-chimeric intron-second transgene (e.g., with a codon-optimized polynucleotide sequence)-SV40 pA-3′, optionally wherein the first transgene and the human TnT promoter are in a reverse orientation, and the second transgene and the chicken TnT promoter are in a forward orientation;
(vi) 5′-human TnT promoter-CMV intron-first transgene-WPRE-bGHpA-pSV40 pA-second transgene (e.g., with a codon-optimized polynucleotide sequence)-chimeric intron-chicken TnT promoter-3′, optionally wherein the first transgene and the human TnT promoter are in a forward orientation, and the second transgene and the chicken TnT promoter are in a reverse orientation; (vii) 5′-huma TnT promoter-CMV intron-first transgene-WPRE-bGHpA-chicken TnT promoter-chimeric intron-second transgene (e.g., with a codon-optimized polynucleotide sequence)-SV40 pA-3′, optionally wherein the first transgene, the human TnT promoter, the second transgene and the chicken TnT promoter are in a forward orientation; and
(viii) 5′-bGHpA-WPRE-first transgene-CMV intron-human TnT promoter-chicken TnT promoter-chimeric intron-second transgene (e.g., with a codon-optimized polynucleotide sequence)-SV40 pA-3′, optionally wherein the first transgene and the human TnT promoter are in a reverse orientation, and the second transgene and the chicken TnT promoter are in a forward orientation.

In some embodiments, the expression cassette is a recombinant expression cassette.

In some aspects, provided herein is a recombinant vector comprising any of the expression cassettes described herein. In some embodiments, the vector is a viral vector. In some embodiments, the vector is a non-viral vector.

In some aspects, provided herein is a recombinant adeno-associated virus (rAAV) virion, comprising a capsid protein and a viral genome comprising any of the expression cassettes described herein, wherein the expression cassette is flanked by inverted terminal repeats (ITRs). In some embodiments, the ITRs share at least 90%, 95%, 96%, 97%, 98%, or 99% identity to one or more of SEQ ID NO: 14 and SEQ ID NO: 15. In some embodiments, the ITRs share at least 90%, 95%, 96%, 97%, 98%, or 99% identity to one or more of SEQ ID NO: 14 and SEQ ID NO: 15. In some embodiments, the capsid protein shares at least 98%, at least 99%, or 100% identity to an AAV9 capsid protein (SEQ ID NO: 143). In some embodiments, the capsid protein shares at least 98%, at least 99%, or 100% identity to an AAV5 capsid protein (SEQ ID NO: 144). In some embodiments, the capsid protein is a chimeric capsid protein. In some embodiments, the capsid protein is an AAV5/AAV9 chimeric capsid protein. In some embodiments, the capsid protein is selected from any one of SEQ ID NOs: 145-200.

In some aspects, provided herein is a pharmaceutical composition comprising any of the vectors described herein or any of the rAAV virions described herein, and a pharmaceutically acceptable carrier.

In some aspects, provided herein is a kit comprises any of the pharmaceutical compositions described herein, or any components of such pharmaceutical compositions (e.g., a vector or an rAAV virion).

In some aspects, provided herein is a method of increasing expression of a polypeptide in a cardiac cell or cardiac tissue comprising contacting a cell with any vector described herein, any rAAV virion described herein, or any pharmaceutical composition described herein. In some embodiments, the cardiac cell is a cardiomyocyte. In some embodiments, the cardiac tissue is heart tissue. In some embodiments, the polypeptide expression is increased between about 1.5-fold and 150-fold. In some embodiments, the polypeptide expression is increased at least 2 fold, at least 5 fold, at least 10 fold, at least 25 fold, at least 50 fold, at least 75 fold, or at least 100 fold. In some embodiments, the contacting is in vitro. In some embodiments, the contacting is in vivo.

In some aspects, provided herein is a method of increasing polypeptide expression in a subject comprising administering to the subject any vector described herein, any rAAV virion described herein, or any pharmaceutical composition described herein. In some embodiments, the subject is a mammal. In some embodiments, the subject is a human. In some embodiments, following administering, the polypeptide expression is increased in the heart of the subject. In some embodiments, the subject being treated has a heart disease or is at risk of a heart disease. In some embodiments, the subject being treated has borderline or reduced ejection fraction. In some embodiments, the subject being treated has normal ejection fraction. In some embodiments, wherein the subject being treated has a genetic mutation associated with a heart disease (e.g., a mutation in a PLN gene). In some embodiments, the subject has a low or undetectable level of expression of the polypeptide encoded by the transgene, compared to a healthy subject.

In some aspects, provided herein is a method of treating or preventing a heart disease or disorder in a subject in need thereof comprising administering to the subject any vector described herein, any rAAV virion described herein, or any pharmaceutical composition described herein. In some embodiments, the subject being treated has a heart disease or disorder. In some embodiments, the subject being treated is a risk of developing a heart disease or disorder. In some embodiments, the heart disease or disorder is cardiomyopathy. In some embodiments, the cardiomyopathy is dilated cardiomyopathy. In some embodiments, the heart disease or disorder is myocardial infarction. In some embodiments, the myocardial infarction is chronic myocardial infarction. In some embodiments, the subject has an inherited risk allele for a heart disease or disorder. In some embodiments, the subject has an inherited risk allele for a heart disease or disorder due to a genetic mutation. In some embodiments, the subject has an inherited risk allele for a heart disease or disorder due to a genetic mutation in a PLN gene (for example, one or more mutations in the PLN gene described herein or known in the art). In some embodiments, the heart disease or disorder is with reduced ejection fraction (HFrEF). In some embodiments, the heart disease of disorder is with preserved ejection fraction (HFpEF). In some embodiments, the method leads to expression of the polypeptide encoded by the transgene in the heart of the subject. In some embodiments, the method leads to expression of the polypeptide encoded by the transgene in cardiomyocytes of the subject. In some embodiments, the method causes no detectable expression of the polypeptide encoded by the transgene in the muscles of the subject except the heart, in the liver of the subject, and/or in the cardiac fibroblasts of the subject. In some embodiments, the method improves one or more measures of cardiac function, optionally fraction shortening and/or left ventricular internal dimension (LVID). In some embodiments, the improvement in cardiac function is observed at or later than week 2, week 4, week 6, week 8, week 10, week 12, week 14, week 16, week 18, week 20, week 22, and/or week 24, after the administering. In some embodiments, the administering is systemic administration. In some embodiments, the systemic administration is selected from intravenous or intracoronary injection. In some embodiments, when an rAAV virion is administered, it is administered as a unit dose. In some embodiments, the unit dose comprises about 3×10¹⁴ vg/kg or less, about 2×10¹⁴ vg/kg or less, about 1×10¹⁴ vg/kg or less, about 9×10¹³ vg/kg or less, about 8×10¹³ vg/kg or less, about 7×10¹³ vg/kg or less, about 6×10¹³ vg/kg or less, about 5×10¹³ vg/kg or less, about 4×10¹³ vg/kg or less, about 3×10¹³ vg/kg or less, about 2×10¹³ vg/kg or less, or about 1×10¹³ vg/kg or less. In some embodiments, the subject being treated is a mammal. In some embodiments, the subject being treated is a human.

In one aspect, the disclosure provides a recombinant adeno-associated virus (rAAV) virion, comprising a capsid protein and a viral genome comprising an expression cassette comprising a polynucleotide sequence encoding a dwarf open reading frame (DWORF) polypeptide operatively linked to a promoter, the expression cassette flanked by inverted terminal repeats.

In some embodiments, the DWORF polypeptide shares at least 90%, 95%, 96%, 97%, 98%, or 99% identity to a sequence selected from SEQ ID NOs: 1, 3, 4, 7, 9, 23, and 43. In some embodiments, the DWORF polypeptide is selected from SEQ ID NOs: 1, 3, 4, 7, 9, 23, and 43.

In some embodiments, the promoter is a chicken cTnT promoter. In some embodiments, the chicken cTnT promoter shares at least 90%, 95%, 96%, 97%, 98%, or 99% identity to SEQ ID NO: 11. In some embodiments, the chicken cTnT promoter comprises SEQ ID NO: 11. In some embodiments, the promoter is a human cTnT promoter. In some embodiments, the human cTnT promoter shares at least 90%, 95%, 96%, 97%, 98%, or 99% identity to SEQ ID NO: 12 or SEQ ID NO: 13. In some embodiments, the human cTnT promoter comprises SEQ ID NO: 12 or SEQ ID NO: 13.

In some embodiments, the expression cassette further comprises one or more enhancers. In some embodiments, the enhancer the one or more enhancers are selected from a ACTC1 cardiac enhancer and a αMHC enhancer. In some embodiments, the ACTC1 cardiac enhancer shares at least 90%, 95%, 96%, 97%, 98%, or 99% identity to SEQ ID NO: 78. In some embodiments, the ACTC1 cardiac enhancer comprises SEQ ID NO: 78. In some embodiments, the αMHC enhancer shares at least 90%, 95%, 96%, 97%, 98%, or 99% identity to SEQ ID NO: 79. In some embodiments, the αMHC enhancer comprises SEQ ID NO: 79.

In some embodiments, the expression cassette further comprises an intron. In some embodiments, the intron is selected from a CMV intron and a chimeric intron. In some embodiments, the CMV intron shares at least 90%, 95%, 96%, 97%, 98%, or 99% identity to SEQ ID NO: 80. In some embodiments, the CMV intron comprises SEQ ID NO: 80. In some embodiments, the chimeric intron shares at least 90%, 95%, 96%, 97%, 98%, or 99% identity to SEQ ID NO: 81. In some embodiments, the chimeric intron comprises SEQ ID NO: 81.

In some embodiments, the expression cassette further comprises a WPRE sequence. In some embodiments, the WPRE sequence shares at least 90%, 95%, 96%, 97%, 98%, or 99% identity to SEQ ID NO: 26. In some embodiments, the WPRE sequence comprises SEQ ID NO: 26.

In some embodiments, the expression cassette further comprises a polyadenylation sequence. In some embodiments, the polyadenylation sequence is selected from a BGH polyadenylation sequence and a SV40 polyadenylation sequence. In some embodiments, the BGH polyadenylation sequence shares at least 90%, 95%, 96%, 97%, 98%, or 99% identity to SEQ ID NO: 27. In some embodiments, the BGH polyadenylation sequence comprises SEQ ID NO: 27. In some embodiments, the SV40 polyadenylation sequence shares at least 90%, 95%, 96%, 97%, 98%, or 99% identity to SEQ ID NO: 28. In some embodiments, the SV40 polyadenylation sequence comprises SEQ ID NO: 28.

In some embodiments, the expression cassette is flanked by ITRs. In some embodiments, the ITRs share at least 90%, 95%, 96%, 97%, 98%, or 99% identity to one or more of SEQ ID NO: 14 and SEQ ID NO: 15. In some embodiments, the ITRs comprise one or more of SEQ ID NO: 14 and SEQ ID NO: 15.

In some embodiments, the expression cassette comprises a single promoter. In some embodiments, the expression cassette comprises two promoters. In some embodiments, the expression cassette comprises a single copy a sequence encoding the DWORF polypeptide. In some embodiments, the expression cassette comprises two copies of a sequence encoding the DWORF polypeptide. In some embodiments, where the expression cassette comprises two copies of a sequence encoding the DWORF polypeptide, the two “copies” are not identical. While not being bound by any theory, using two nucleic acid sequences encoding a polypeptide that are not identical may prevent DNA recombination within the vector. In some embodiments, the expression cassette comprises one copy that has the original DNA sequence encoding the DWORF polypeptide and one copy that has a codon optimized DNA sequence encoding the DWORF polypeptide. In some embodiments, the expression cassette comprises two copies of a sequence encoding the DWORF polypeptide, wherein one copy is codon-optimized and one copy is not codon optimized. In some embodiments, the expression cassette comprises one, two, three, or four enhancers. In some embodiments, the expression cassette comprises one or two introns. In some embodiments, the expression cassette comprises one or two WPRE sequences. In some embodiments, the expression cassette comprises one or two polyadenylation sequences.

In some embodiments, the expression cassette comprises about 3.2 kb, about, about 3.3 kb, about 3.4 kb, about 3.5 kb, about 3.6 kb, about 3.7 kb, or less. In some embodiments, the expression cassette comprises about 1.9 kb, about 2.1 kb, about 2.2 kb, about 2.3 kb, about 2.4 kb, about 2.5 kb, about 2.6 kb, about 2.7 kb, about 2.8 kb, about 2.9 kb, about 3.0 kb, about 3.1 kb, about 3.2 kb, or more.

In some embodiments, the expression cassette comprises a polynucleotide sequence that shares at least 75%, 80%, 90%, 95%, 96%, 97%, 98%, 99%, or 100% identity to any one of SEQ ID NOs: 20-24 or SEQ ID NOs: 45-75. In some embodiments, wherein the expression cassette comprises any one of SEQ ID NOs: 20-24 or SEQ ID NOs: 45-75. In some embodiments, the expression cassette comprises a polynucleotide sequence that shares at least 75%, 80%, 90%, 95%, 96%, 97%, 98%, or 99% identity to SEQ ID NO: 61. In some embodiments, the expression cassette comprises SEQ ID NO: 61. In some embodiments, the expression cassette comprises a polynucleotide sequence that shares at least 75%, 80%, 90%, 95%, 96%, 97%, 98%, or 99% identity to SEQ ID NO: 62. In some embodiments, the expression cassette comprises SEQ ID NO: 62. In some embodiments, the expression cassette comprises a polynucleotide sequence that shares at least 75%, 80%, 90%, 95%, 96%, 97%, 98%, or 99% identity to SEQ ID NO: 63. In some embodiments, the expression cassette comprises SEQ ID NO: 63.

In some embodiments, the capsid protein shares at least 98%, at least 99%, or 100% identity to an AAV9 capsid protein (SEQ ID NO: 143). In some embodiments, the capsid protein shares at least 98%, at least 99%, or 100% identity to an AAV5 capsid protein (SEQ ID NO: 144). In some embodiments, the capsid protein is a chimeric capsid protein. In some embodiments, the capsid protein is an AAV5/AAV9 chimeric capsid protein. In some embodiments, the capsid protein is selected from any one of SEQ ID NOs: 145-200.

In one aspect, the disclosure provides an expression cassette comprising polynucleotide sequence encoding a dwarf open reading frame (DWORF) polypeptide operatively linked to a promoter. In some embodiments, the DWORF polypeptide shares at least 75%, 80%, 90%, 95%, 96%, 97%, 98%, or 99% identity to SEQ ID NOs: 1, 3, 4, 7, 9, 23, and 43. In some embodiments, the DWORF polypeptide is selected from SEQ ID NOs: 1, 3, 4, 7, 9, 23, and 43.

In some embodiments, the promoter is a chicken cTnT promoter. In some embodiments, the chicken cTnT promoter shares at least 90%, 95%, 96%, 97%, 98%, or 99% identity to SEQ ID NO: 11. In some embodiments, the chicken cTnT promoter comprises SEQ ID NO: 11. In some embodiments, the promoter is a human cTnT promoter. In some embodiments, the human cTnT promoter shares at least 90%, 95%, 96%, 97%, 98%, or 99% identity to SEQ ID NO: 12 or SEQ ID NO: 13. In some embodiments, the human cTnT promoter comprises SEQ ID NO: 12 or SEQ ID NO: 13.

In some embodiments, the expression cassette further comprises one or more enhancers. In some embodiments, the enhancer the one or more enhancers are selected from a ACTC1 cardiac enhancer and a αMHC enhancer. In some embodiments, the ACTC1 cardiac enhancer shares at least 90%, 95%, 96%, 97%, 98%, or 99% identity to SEQ ID NO: 78. In some embodiments, the ACTC1 cardiac enhancer comprises SEQ ID NO: 78. In some embodiments, the αMHC enhancer shares at least 90%, 95%, 96%, 97%, 98%, or 99% identity to SEQ ID NO: 79. In some embodiments, the αMHC enhancer comprises SEQ ID NO: 79.

In some embodiments, the expression cassette further comprises an intron. In some embodiments, the intron is selected from a CMV intron and a chimeric intron. In some embodiments, the CMV intron shares at least 90%, 95%, 96%, 97%, 98%, or 99% identity to SEQ ID NO: 80. In some embodiments, the CMV intron comprises SEQ ID NO: 80. In some embodiments, the chimeric intron shares at least 90%, 95%, 96%, 97%, 98%, or 99% identity to SEQ ID NO: 81. In some embodiments, the chimeric intron comprises SEQ ID NO: 81.

In some embodiments, the expression cassette further comprises a WPRE sequence. In some embodiments, the WPRE sequence shares at least 90%, 95%, 96%, 97%, 98%, or 99% identity to SEQ ID NO: 26. In some embodiments, the WPRE sequence comprises SEQ ID NO: 26.

In some embodiments, the expression cassette further comprises a polyadenylation sequence. In some embodiments, the polyadenylation sequence is selected from a BGH polyadenylation sequence and a SV40 polyadenylation sequence. In some embodiments, the BGH polyadenylation sequence shares at least 90%, 95%, 96%, 97%, 98%, or 99% identity to SEQ ID NO: 27. In some embodiments, the BGH polyadenylation sequence comprises SEQ ID NO: 27. In some embodiments, the SV40 polyadenylation sequence shares at least 90%, 95%, 96%, 97%, 98%, or 99% identity to SEQ ID NO: 28. In some embodiments, the SV40 polyadenylation sequence comprises SEQ ID NO: 28.

In some embodiments, the expression cassette is flanked by ITRs. In some embodiments, the ITRs share at least 90%, 95%, 96%, 97%, 98%, or 99% identity to one or more of SEQ ID NO: 14 and SEQ ID NO: 15. In some embodiments, the ITRs comprise one or more of SEQ ID NO: 14 and SEQ ID NO: 15.

In some embodiments, the expression cassette comprises a single promoter. In some embodiments, the expression cassette comprises two promoters. In some embodiments, the expression cassette comprises a single copy a sequence encoding the DWORF polypeptide. In some embodiments, the expression cassette comprises two copies of a sequence encoding the DWORF polypeptide. In some embodiments, the expression cassette comprises one, two, three, or four enhancers. In some embodiments, the expression cassette comprises one or two introns. In some embodiments, the expression cassette comprises one or two WPRE sequences. In some embodiments, the expression cassette comprises one or two polyadenylation sequences.

In some embodiments, the expression cassette comprises about 3.2 kb, about, about 3.3 kb, about 3.4 kb, about 3.5 kb, about 3.6 kb, about 3.7 kb, or less. In some embodiments, the expression cassette comprises about 1.9 kb, about 2.1 kb, about 2.2 kb, about 2.3 kb, about 2.4 kb, about 2.5 kb, about 2.6 kb, about 2.7 kb, about 2.8 kb, about 2.9 kb, about 3.0 kb, about 3.1 kb, about 3.2 kb, or more.

In some embodiments, the expression cassette comprises a polynucleotide sequence that shares at least 75%, 80%, 90%, 95%, 96%, 97%, 98%, 99%, or 100% identity to any one of SEQ ID NOs: 20-24 or SEQ ID NOs: 45-75. In some embodiments, the expression cassette comprises any one of SEQ ID NOs: 20-24 or SEQ ID NOs: 45-75. In some embodiments, the expression cassette comprises a polynucleotide sequence that shares at least 75%, 80%, 90%, 95%, 96%, 97%, 98%, or 99% identity to SEQ ID NO: 61. In some embodiments, the expression cassette comprises SEQ ID NO: 61. In some embodiments, the expression cassette comprises a polynucleotide sequence that shares at least 75%, 80%, 90%, 95%, 96%, 97%, 98%, or 99% identity to SEQ ID NO: 62. In some embodiments, the expression cassette comprises SEQ ID NO: 62. In some embodiments, the expression cassette comprises a polynucleotide sequence that shares at least 75%, 80%, 90%, 95%, 96%, 97%, 98%, or 99% identity to SEQ ID NO: 63. In some embodiments, the expression cassette comprises SEQ ID NO: 63.

In some embodiments, the expression cassette comprises a 5′ inverted terminal repeat and a 3′ inverted terminal repeat.

In one aspect, the disclosure provides a pharmaceutical composition comprising the rAAV virion disclosed herein and an pharmaceutically acceptable diluent. In another aspect, the disclosure provides a kit comprising a pharmaceutical composition provided herein.

In one aspect, the disclosure provides a method of increasing DWORF expression in a cell comprising contacting a cell with the rAAV virion disclosed herein or the pharmaceutical composition disclosed herein. In some embodiments, the cell is a cardiac cell. In some embodiments, the cardiac cell is a cardiomyocyte. In some embodiments, DWORF expression is increased between about 1.5-fold and 150-fold. In some embodiments, the contacting is in vitro. In some embodiments, the contacting is in vivo.

In one aspect, the disclosure provides a method of increasing DWORF expression in a tissue comprising contacting the tissue with the rAAV virion disclosed herein or the pharmaceutical composition disclosed herein. In some embodiments, the tissue is cardiac tissue. In some embodiments, DWORF expression is increased between about 1.5-fold and 150-fold. In some embodiments, the contacting is in vitro. In some embodiments, the contacting is in vivo.

In one aspect, the disclosure provides a method of increasing DWORF expression in an organ comprising contacting the organ with the rAAV virion disclosed herein or the pharmaceutical composition disclosed herein. In some embodiments, DWORF expression is increased between about 1.5-fold and 150-fold.

In some embodiments, the organ is a heart. In some embodiments, the heart is diseased or is at risk of heart disease. In some embodiments, the heart has reduced or borderline ejection fraction. In some embodiments, the heart has a normal ejection fraction.

In some embodiments, the heart comprises a genetic mutation associated with a heart disease. In some embodiments, the genetic mutation is a PLN mutation. In some embodiments, the heart has low or undetectable DWORF expression compared to a healthy heart. In some embodiments, the contacting is in vitro. In some embodiments, the contacting is in vivo.

In one aspect, the disclosure provides a method of increasing DWORF expression in a subject comprising administering to the subject the rAAV virion disclosed herein or the pharmaceutical composition disclosed herein. In some embodiments, the subject is an animal. In some embodiments, the subject is a human. In some embodiments, DWORF expression is increased in the heart of the subject. In some embodiments, the subject has a heart disease or is at risk of a heart disease. In some embodiments, the subject has borderline or reduced ejection fraction. In some embodiments, the subject has normal ejection fraction. In some embodiments, the subject has a genetic mutation associated with a heart disease. In some embodiments, the genetic mutation is a PLN mutation. In some embodiments, the subject has a low or undetectable level of DWORF expression compared to a healthy subject.

In one aspect, the disclosure provides a method of treating a heart disease or disorder in a subject in need thereof comprising administering to the subject the rAAV virion disclosed herein or the pharmaceutical composition disclosed herein.

In some embodiments, the subject has a heart disease or disorder. In some embodiments, the subject is a risk of developing a heart disease or disorder. In some embodiments, the heart disease or disorder is cardiomyopathy. In some embodiments, the heart disease or disorder is dilated cardiomyopathy. In some embodiments, the heart disease or disorder is myocardial infarction. In some embodiments, the heart disease or disorder is chronic myocardial infarction. In some embodiments, the heart disease or disorder is acute myocardial infarction.

In some embodiments, the subject has an inherited risk allele for a heart disease or disorder. In some embodiments, the inherited risk allele comprises a mutation to the PLN gene. In some embodiments, the mutation to the PLN gene is a PLN promoter mutation. In some embodiments, the mutation to the PLN gene is a PLNL39stop mutation. In some embodiments, the mutation to the PLN gene is a RC9 mutation. In some embodiments, the mutation to the PLN gene is a R9L mutation. In some embodiments, the mutation to the PLN gene is a PLN gene duplication. In some embodiments, the mutation to the PLN gene is a R14del mutation.

In some embodiments, the heart disease or disorder is with reduced ejection fraction (HFrEF). In some embodiments, the heart disease of disorder is with preserved ejection fraction (HFpEF).

In some embodiments, the method causes expression of the DWORF polypeptide in the heart of the subject. In some embodiments, the method causes expression of the DWORF polypeptide in cardiomyocytes.

In some embodiments, the method causes no detectable expression of the DWORF polypeptide in the muscles of the subject except the heart. In some embodiments, the method causes no detectable expression of the DWORF polypeptide in the liver of the subject. In some embodiments, the method causes no detectable expression of the DWORF polypeptide in cardiac fibroblasts.

In some embodiments, the method improves one or more measures of cardiac function, optionally fraction shortening and/or left ventricular internal dimension (LVID). In some embodiments, the improvement in cardiac function is observed at weeks 2 through week 16. In some embodiments, the method reduces cardiac remodeling. In some embodiments, the method counteracts a decrease in DWORF expression in subjects suffering from or at risk of a heart disease.

In some embodiments, the rAAV virion is administered by systemic administration. In some embodiments, the systemic administration is selected from intravenous or intracoronary injection.

In some embodiments, the rAAV is administered as a unit dose. In some embodiments, the unit dose comprises about 3×10¹⁴ vg/kg or less, about 2×10¹⁴ vg/kg or less, about 1×10¹⁴ vg/kg or less, about 9×10¹³ vg/kg or less, about 8×10¹³ vg/kg or less, about 7×10¹³ vg/kg or less, about 6×10¹³ vg/kg or less, about 5×10¹³ vg/kg or less, about 4×10¹³ vg/kg or less, about 3×10¹³ vg/kg or less, about 2×10¹³ vg/kg or less, or about 1×10¹³ vg/kg or less.

In one aspect, the disclosure provides a method of alleviating one or more symptoms of a heart disease or disorder in a subject in need thereof comprising administering the rAAV virion disclosed herein or the pharmaceutical composition disclosed herein.

In one aspect, the disclosure provides a method of improving one or more symptoms of a heart disease or disorder in a subject in need thereof comprising administering the rAAV virion disclosed herein or the pharmaceutical composition disclosed herein.

In one aspect, the disclosure provides a method of preventing one or more symptoms of a heart disease or disorder in a subject in need thereof comprising administering the rAAV virion disclosed herein or the pharmaceutical composition disclosed herein.

In one aspect, the disclosure provides an expression cassette comprising a polynucleotide comprising a 5′ to 3′ arrangement of elements, wherein the elements comprise: i) one or more promoters; ii) optionally one or more enhancers; iii) optionally one or more introns; iv) one or more transgenes; v) optionally one or more WPRE sequences; and vi) optionally one or more polyadenylation sequences, p(A). In some embodiments, the 5′ to 3′ arrangement of elements is selected from: i) 5′-promoter-intron-transgene-WPRE-p(A)-3′; ii) 5′-enhancer-promoter-transgene-WPRE-p(A)-3′; iii) 5′-enhancer-enhancer-promoter-transgene-WPRE-p(A)-3′; iv) 5′-enhancer-enhancer-promoter-intron-transgene-WPRE-p(A)-3′; v) 5′-enhancer-enhancer-promoter-intron-transgene-WPRE-p(A)-3′; vi) 5′-enhancer-promoter-intron-transgene-WPRE-p(A)-p(A)-transgene-intron-promoter-enhancer-3′; vii) 5′-enhancer-promoter-intron-transgene-WPRE-p(A)-enhancer-promoter-intron-transgene-p(A)-3′; viii) 5′-p(A)-WPRE-transgene-intron-promoter-enhancer-enhancer-promoter-intron-transgene-p(A)-3′; ix) 5′-promoter-intron-transgene-WPRE-p(A)-p(A)-transgene-intron-promoter-3′; x) 5′-promoter-intron-transgene-WPRE-p(A)-promoter-intron-transgene-p(A)-3′; and xi) 5′-p(A)-WPRE-transgene-intron-promoter-promoter-intron-transgene-p(A)-3′. In some embodiments, the transgene has an increased expression level compared to a second expression cassette comprising a polynucleotide having an arrangement of elements from 5′ to 3′ comprising: 5′-promoter-transgene-WPRE-p(A)-3′. In some embodiments, the increased expression level is between about 1.5-fold and about 150-fold compared to the second expression cassette.

In one aspect, the disclosure provides a recombinant adeno-associated virus (rAAV) virion, comprising a capsid protein and a viral genome comprising an expression cassette of any one of disclosed herein, the expression cassette flanked by inverted terminal repeats. In some embodiments, the expression cassette comprises a transgene, wherein the transgene encodes a polypeptide use for treating or a preventing a heart disease, or alleviating symptoms associated with a heart disease. In some embodiments, the capsid protein is selected from any one of SEQ ID NOs: 145-200.

### BRIEF DESCRIPTIONS OF DRAWINGS

FIG. 1 shows a diagram of illustrative embodiments of expression cassettes comprising a polynucleotide encoding a promoter, a DWORF polypeptide, a WPRE sequence, and a poly(A) signal sequence flanked by AAV inverted terminal repeats.

FIG. 2 is a graph showing expression of GFP delivered to human induced pluripotent stem cell derived cardiomyocytes in vitro using an embodiment of an expression cassette packaged into an rAAV virion.

FIG. 3 is a graph showing expression of human DWORF polypeptide delivered in vivo in a murine model using an embodiment of an expression cassette packed into an rAAV virion using an AAV9 capsid protein.

FIG. 4A is a graph showing expression of human DWORF in induced pluripotent stem cell derived cardiomyocytes using an embodiment of an expression cassette packaged into an rAAV virion one of several AAV protein capsid proteins described herein.

FIG. 4B is a series of images showing expression of GFP using an embodiment of an expression cassette described herein packaged into an rAAV virion one of several AAV protein capsid proteins described herein.

FIG. 5A is a graph showing DWORF RNA expression in heart tissue from animals treated with rAAV virions containing an embodiment of an expression cassette packaged with one of five chimeric capsid proteins or the AAV9 capsid protein.

FIG. 5B is an immunoblot showing DWORF protein levels in heart tissue from animals treated with rAAV virions containing an embodiment of an expression cassette packaged with one of four chimeric capsid proteins or the AAV9 capsid protein.

FIG. 6A is a graph showing improved ejection fraction in a PLN-R14^{Δ/Δ} mouse model following treatment with an embodiment of an expression cassette packaged into an rAAV virion using AAV9 protein capsid.

FIG. 6B is a graph showing improved fractional shortening in a PLN-R14^{Δ/Δ} mouse model following treatment with an embodiment of an expression cassette packaged into an rAAV virion using AAV9 protein capsid.

FIG. 7A shows a diagram of an illustrative expression cassette orientation comprising a polynucleotide encoding a promoter, a DWORF polypeptide, a WPRE sequence, and a poly(A) signal sequence flanked by AAV inverted terminal repeats.

FIG. 7B shows a diagram of illustrative expression cassette orientations comprising a polynucleotide encoding a promoter, one or more enhancers, an intron, a DWORF polypeptide, a WPRE sequence, and a poly(A) signal sequence flanked by AAV inverted terminal repeats.

FIG. 7C shows a diagram of illustrative expression cassette orientations comprising a polynucleotide encoding a promoter, one or more enhancers, an intron, a DWORF polypeptide, a WPRE sequence, and a poly(A) signal sequence flanked by AAV inverted terminal repeats.

FIG. 8A is a schematic diagram which outlines the strategy for assessing the expression of DWORF in cardiomyocytes mice retro-orbitally injected with AAV9:DWORF constructs containing various regulatory elements and arrangements in vivo.

FIG. 8B is a western blot which demonstrates the expression of DWORF and GAPDH in cardiomyocytes mice retro-orbitally injected with AAV9:DWORF constructs containing various regulatory elements and arrangements in vivo.

FIG. 8C is a chart showing the DWORF expression level in an animal model achieved using a panel of rAAV virions comprising expression cassettes encoding a DWORF polypeptide.

FIG. 9 is a plot showing improved ejection fraction in an animal model of cardiomyopathy treated with a panel of rAAV virions comprising expression cassettes encoding a DWORF polypeptide.

FIG. 10 is a plot showing preserved ejection fraction in an animal model of cardiomyopathy treated with a panel of rAAV virions comprising expression cassettes encoding a DWORF polypeptide.

FIG. 11A is a schematic diagram of DWORF gene therapy efficacy study in the MLP-KO DCM mouse model.

FIGS. 11B and 11C demonstrate that AAV9:DWORF constructs containing novel promoters improve the ejection fraction relative to a saline control in the MLP-KO DCM mouse model.

FIGS. 11D and 11E demonstrate that AAV9:DWORF constructs improved exercise capacity, including running distance and time to exhaustion, in the MLP-KO DCM mouse model 26 weeks post-treatment.

FIG. 12A is a schematic diagram of detailing DWORF gene therapy tolerability study in naïve mice.

FIG. 12B demonstrates that AAV9:pHZ21 is well tolerated in naïve mice up to 2×10¹⁴ vg/kg dose with no difference in body weight, ejection fraction, heart rate, and left ventricular mass (LV mass).

### DETAILED DESCRIPTION

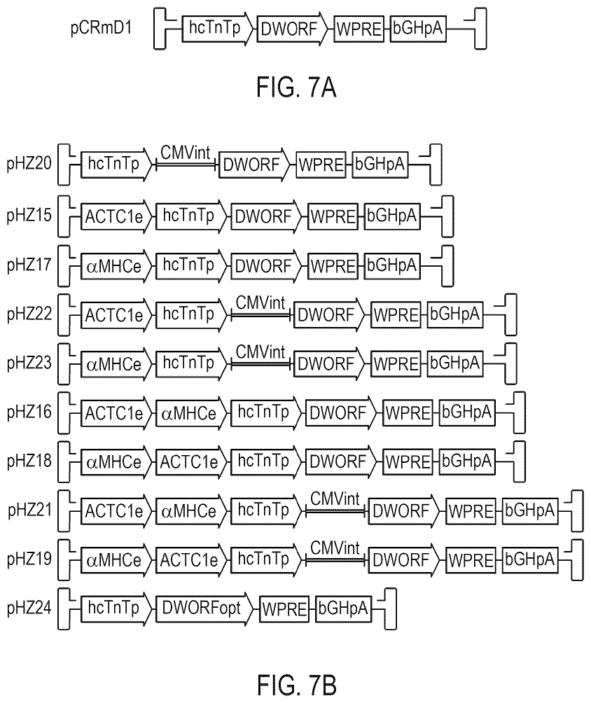

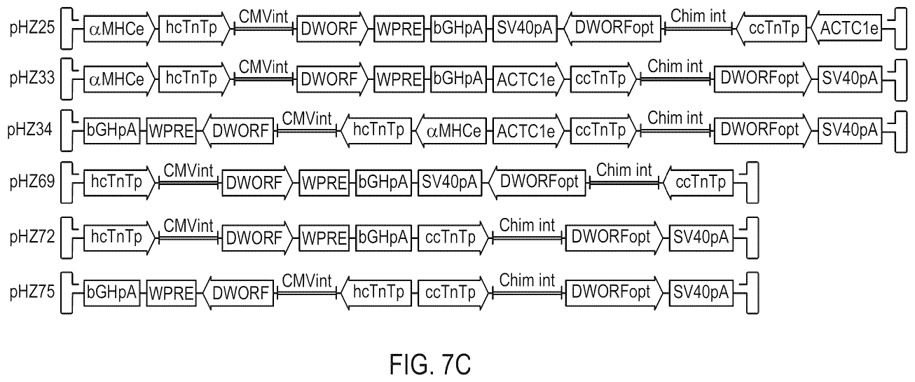

In some aspects, described herein are optimized gene therapy expression cassettes, and their use in the treatment of heart disease. In some aspects, described herein are gene therapy expression cassettes that are able to mediate high expression of transgenes. In some embodiments, described herein are cardiac-specific gene therapy expression cassettes that are able to mediate significantly higher expression of a transgene than can be achieved using a cTnT promoter alone (e.g., a chicken cTnT promoter alone and/or a human cTnT promoter alone) or using the expression cassette depicted in FIG. 7A. In some aspects, described herein are gene therapy expression cassettes that allow to lower the viral load while achieving desired expression of a transgene. In some aspects, described herein are gene therapy expression cassettes that allow to achieve durable expression of a transgene. In some embodiments, described herein are gene therapy expression cassettes that allow to achieve expression of a transgene for at least, or more than, 12 weeks, 16 weeks, 18 weeks, 20 weeks, 22 weeks, 24 weeks, or 26 weeks, after administration of a gene therapy expression cassette comprising the transgene to a subject. In some embodiments, described herein are gene therapy expression cassettes that allow to achieve expression of a transgene for at least, or more than, 24 weeks or at least 6 months after administration of a gene therapy expression cassette comprising the transgene to a subject. In some aspects, administration of a gene therapy expression cassette described herein to a subject results in one or more improvements in cardiac function (e.g., an improvement in ejection fraction or an improvement in exercise capacity). In some embodiments, administration of a gene therapy expression cassette described herein to a subject results in a durable improvement in cardiac function (e.g., a durable improvement in ejection fraction or a durable improvement in exercise capacity). In some embodiments, administration of a gene therapy expression cassette described herein to a subject results in one or more improvements in cardiac function for at least, or more than, 12 weeks, 16 weeks, 18 weeks, 20 weeks, 22 weeks, 24 weeks, or 26 weeks after the administration. In some embodiments, administration of a gene therapy expression cassette described herein to a subject results in one or more improvements in cardiac function for at least, or more than, 24 weeks or at least 6 months after the administration. In some embodiments, a gene therapy expression cassette described herein allows for cardiac cell-specific expression of a transgene (e.g., cardiomyocyte-specific expression of a transgene).

In some aspects, the present disclosure provides a viral or a non-viral vector comprising an expression cassette encoding a gene product, and methods of use thereof. In some embodiments, the expression cassettes described herein comprise a polynucleotide encoding a gene product operably linked to a cardiac cell-specific promoter and/or enhancer (such as any combination of cardiac cell-specific promoters and enhancers described herein, in any orientation as described herein). In some embodiments, the expression cassettes described herein comprise two copies of a polynucleotide encoding a gene product and a cardiac cell-specific promoter (such as any combination of such sequences, in any orientation as described herein). In some embodiments, the expression cassettes described herein comprise a polynucleotide encoding a gene product operably linked to a cardiac cell-specific promoter and/or enhancer (such as one promoter, or any combination of cardiac cell-specific promoters and enhancers described herein, in any orientation as described herein), a WPRE sequence, and/or one or two copies of a polyA sequence (such as any combination of such sequences, in any orientation as described herein). In some embodiments, the expression cassettes described herein comprise a polynucleotide encoding a gene product operably linked to a cardiac cell-specific promoter and/or an intron. In some embodiments, the expression cassettes described herein comprise one or two copies of a polynucleotide encoding a gene product, one or two copies of a cardiac cell-specific promoter, one, two or more copies of a cardiac-specific enhancer, and/or one or more intron sequences (such as any combination of such sequences, in any orientation as described herein). In some embodiments, the expression cassettes described herein comprise one or two copies of a polynucleotide encoding a gene product, one or two copies of a cardiac cell-specific promoter, one, two or more copies of a cardiac-specific enhancer, one or more intron sequences (such as any combination of such sequences, in any orientation as described herein). a WPRE sequence, and one or two copies of a polyA sequence (such as any combination of such sequences, in any orientation as described herein). In some embodiments, the vectors comprising the expression cassettes described herein may, for example, transduce cardiac cells. In some embodiments, targeted cardiac cells express the gene product, e.g., provide a high level of expression of the gene product. In some aspects, the present disclosure provides pharmaceutical compositions comprising the vectors described herein. In some aspects, the disclosure provides methods for treating a subject diagnosed with or at risk of a heart disease (e.g., cardiomyopathy) using the vectors and pharmaceutical compositions of the disclosure.

In some aspects, the present disclosure provides recombinant adeno-associated virus (rAAV) virions as a vector for the expression cassette described herein.

Abnormal calcium handling is a universal characteristic of cardiomyopathy, and reduced sarco/endoplasmic reticulum calcium ATPase (SERCA) activity plays a central role in both the initiation and progression of the disease. SERCA is a calcium pump that promotes the uptake, maintenance, and cycling of Ca²⁺, ions in cardiac cells, such as cardiomyocytes. SERCA activity is regulated by an inhibitory peptide, phospholamban. There is significant interest in increasing the activity of SERCA by increasing the abundance of a polypeptide called Dwarf Open Reading Frame (DWORF) that enhances SERCA activity through its direct displacement of the SERCA inhibitory peptide phospholamban. Contacting SERCA with DWORF is a strategy for increasing SERCA activity in a cell.

In some aspects, the present disclosure provides recombinant adeno-associated virus (rAAV) virions comprising a polynucleotide encoding a DWORF polypeptide, or a functional variant thereof, and methods of use thereof. In some embodiments, the rAAV virions described herein comprise a polynucleotide encoding a DWORF polypeptide, or a functional variant thereof, operably linked to a cardiac cell-specific promoter and/or enhancer (such as any combination of cardiac cell-specific promoters and enhancers described herein, in any orientation as described herein). In some embodiments, the rAAV virions described herein comprise one or two copies of a polynucleotide encoding a DWORF polypeptide, or a functional variant thereof, a WPRE sequence, and one or two copies of a polyA sequence (such as any combination of such sequences, in any orientation as described herein). In some embodiments, the rAAV virions described herein comprise a polynucleotide encoding a DWORF polypeptide operably linked to a cardiac cell-specific promoter and/or an intron. In some embodiments, the rAAV virions described herein comprise one or two copies of a polynucleotide encoding DWORF, one or two copies of a cardiac cell-specific promoter, one, two or more copies of a cardiac-specific enhancer, and/or one or more intron sequences (such as any combination of such sequences, in any orientation as described herein). In some embodiments, the rAAV virions described herein comprise one or two copies of a polynucleotide encoding DWORF, one or two copies of a cardiac cell-specific promoter, one, two or more copies of a cardiac-specific enhancer, one or more intron sequences (such as any combination of such sequences, in any orientation as described herein). a WPRE sequence, and one or two copies of a polyA sequence (such as any combination of such sequences, in any orientation as described herein). In some embodiments, the rAAV virions described herein may, for example, transduce cardiac cells with a polynucleotide with a sequence encoding DWORF polypeptide operatively linked to a cardiac cell-specific promoter region into the host cell genome. In some embodiments, targeted cardiac cells express the DWORF polypeptide and may have increased SERCA activity. Also provided in the disclosure are pharmaceutical compositions comprising the rAAV virions described herein. In an aspect, the disclosure provides methods for treating a subject diagnosed with or at risk of cardiomyopathy using the rAAV virions and pharmaceutical compositions of the disclosure.

### TERMINOLOGY

Unless the context indicates otherwise, the features of the invention can be used in any combination. Any feature or combination of features set forth can be excluded or omitted. Certain features of the invention, which are described in separate embodiments may also be provided in combination in a single embodiment. Features of the invention, which are described in a single embodiment may also be provided separately or in any suitable sub-combination.

Generally, technical and scientific terms used herein have the same meaning as commonly understood by one of ordinary skill in the art to which this invention belongs. The detailed description is divided into sections only for the reader's convenience and disclosure found in any section may be combined with that in another section.

The practice of the present disclosure will employ, unless otherwise indicated, conventional techniques of tissue culture, immunology, molecular biology, cell biology and recombinant DNA, which are within the skill of the art. See, e.g., Sambrook and Russell eds. (2001) Molecular Cloning: A Laboratory Manual, 3^{rd} edition; Ausubel et al. eds. (2007) Current Protocols in Molecular Biology; Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; IRL Press (1986) Immobilized Cells and Enzymes; Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (2002) Cold Spring Harbor Laboratory Press; Sohail (2004) Gene Silencing by RNA Interference: Technology and Application (CRC Press); and Sell (2013) Stem Cells Handbook.

The conjunction “and/or” means both “and” and “or,” and lists joined by “and/or” encompasses all possible combinations of one or more of the listed items.

As used herein, the term “about,” when used to modify a numeric value, indicates that deviations of up to 10% above and below the numeric value remain within the intended meaning of the recited value.

“AAV” is an abbreviation for adeno-associated virus. The term covers all subtypes of AAV, except where a subtype is indicated, and to both naturally occurring and recombinant forms. The abbreviation “rAAV” refers to recombinant adeno-associated virus. “AAV” includes AAV or any subtype. “AAV5” refers to AAV subtype 5. “AAV9” refers to AAV subtype 9. The genomic sequences of various serotypes of AAV, as well as the sequences of the native inverted terminal repeats (ITRs), Rep proteins, and capsid subunits may be found in the literature or in public databases such as GenBank. See, e.g., GenBank Accession Numbers NC_002077 (AAV1), AF063497 (AAV1), NC_001401 (AAV2), AF043303 (AAV2), NC_001729 (AAV3), NC_001829 (AAV4), U89790 (AAV4), NC_006152 (AAV5), AF513851 (AAV7), AF513852 (AAV8), NC_006261 (AAV8), and AY530579 (AAV9). Publications describing AAV include Srivistava et al. (1983) J. Virol. 45:555; Chiorini et al. (1998) J. Virol. 71:6823; Chiorini et al. (1999) J. Virol. 73:1309; Bantel-Schaal et al. (1999) J. Virol. 73:939; Xiao et al. (1999) J. Virol. 73:3994; Muramatsu et al. (1996) Virol. 221:208; Shade et al. (1986) J. Virol. 58:921; Gao et al. (2002) Proc. Nat. Acad. Sci. USA 99: 11854; Moris et al. (2004) Virology 33:375-383; Int'l Pat. Publ Nos. WO2018/222503A1, WO2012/145601A2, WO2000/028061A2, WO1999/61601A2, and WO1998/11244A2; U.S. patent application Ser. Nos. 15/782,980 and 15/433,322; and U.S. Pat. Nos. 10,036,016, 9,790,472, 9,737,618, 9,434,928, 9,233,131, 8,906,675, 7,790,449, 7,906,111, 7,718,424, 7,259,151, 7,198,951, 7,105,345, 6,962,815, 6,984,517, and 6,156,303.

An “rAAV virion” refers to a viral particle including at least one viral capsid protein (e.g. VP1) and an encapsidated rAAV vector (or fragment thereof).

An “infectious” virion or viral particle is one that comprises a competently assembled viral capsid and is capable of delivering a polynucleotide component into a cell for which the virion is tropic.

“Packaging” refers to a series of intracellular events that result in the assembly of an rAAV virion including encapsidation of the rAAV vector. AAV “rep” and “cap” genes refer to polynucleotide sequences encoding replication and encapsidation proteins of adeno-associated virus. AAV rep and cap are referred to herein as AAV “packaging genes.” Packaging requires either a helper virus itself or, more commonly in recombinant systems, helper virus function supplied by a helper-free system (i.e. one or more helper plasmids). A “helper virus” for AAV refers to a virus that allows AAV (e.g. wild-type AAV) to be replicated and packaged by a mammalian cell. The helper viruses may be an adenovirus, herpesvirus or poxvirus, such as vaccinia.

The term “inverted terminal repeats” or “ITRs” as used herein refers to AAV viral cis-elements named so because of their symmetry. These elements are essential for efficient multiplication of an AAV genome. In some embodiments, the minimal elements indispensable for ITR function are a Rep-binding site and a terminal resolution site plus a variable palindromic sequence allowing for hairpin formation.

The terms “parental capsid” or “parental sequence” refer to a reference sequence from which a particle capsid or sequence is derived. Unless otherwise specified, parental sequence refers to the sequence of the wild-type capsid protein of the same serotype as the engineered capsid protein.

“Recombinant,” as applied to a polynucleotide means that the polynucleotide is distinct from a polynucleotide found in nature (e.g., the polynucleotide is the product of various combinations of cloning, restriction or ligation steps, and other procedures, or the polynucleotide is assembled from synthetic oligonucleotides. A “recombinant” protein is a protein produced from a recombinant polypeptide. A recombinant virion is a virion that comprises a recombinant polynucleotide and/or a recombinant protein, e.g. a recombinant capsid protein.

As used herein, the term “percent sequence identity,” and the term “identity” when it is used to refer to % sequence identity, with respect to a reference nucleic acid or amino acid sequence is the percentage of nucleic acid bases or amino acid residues in a candidate sequence that are identical with the nucleic acid bases or amino acid residues in the reference sequence, respectively, after aligning the sequences and introducing gaps, if necessary, to achieve the maximum percent sequence identity. Methods of sequence alignment are well known in the art. Sequences can be aligned using various computer programs, such BLAST, available at ncbi.nlm.nih.gov. Alignments can be made using publicly available computer software such as BLASTp, BLASTn, BLAST-2, ALIGN or MegAlign Pro (DNASTAR) software. Other techniques for alignment are described in Methods in Enzymology, vol. 266: Computer Methods for Macromolecular Sequence Analysis (1996); and Meth. Mol. Biol. 70: 173-187 (1997); J. Mol. Biol. 48: 44. Skill artisans are capable of choosing an appropriate alignment method depending on various factors including sequence length, divergence, and the presence of absence of insertions or deletions with respect to the reference sequence.

The terms “operably linked” and “operatively linked” refer to a nucleic acid sequence placed into a functional relationship with another nucleic acid sequence. These terms, as used herein, have a meaning commonly known in the art. For example, a promoter is operably linked to a gene when that promoter is placed in a location that permits that promoter to initiate transcription of that gene. An enhancer is operably linked to a gene when that enhancer, when bound by an appropriate transcription factor, can regulate (e.g., enhance) expression of that gene.

“Treatment,” “treating,” and “treat” are defined as acting upon a disease, disorder, or condition with an agent to reduce or ameliorate harmful or any other undesired effects of the disease, disorder, or condition and/or its symptoms.

As used herein the term “effective amount” and the like in reference to an amount of a composition refers to an amount that is sufficient to induce a desired physiologic outcome (e.g., treatment of a disease). An effective amount can be administered in one or more administrations, applications or dosages. Such delivery is dependent on a number of variables including the time period which the individual dosage unit is to be used, the bioavailability of the composition, the route of administration, etc. It is understood, however, that specific amounts of the compositions (e.g., rAAV virions) for any particular subject depends upon a variety of factors including the activity of the specific agent employed, the age, body weight, general health, sex, and diet of the subject, the time of administration, the rate of excretion, the composition combination, severity of the particular disease being treated and form of administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “individual,” “subject,” and “patient” are used interchangeably herein, and refer to a mammal, including, but not limited to, human and non-human primates (e.g., simians); mammalian sport animals (e.g., horses); mammalian farm animals (e.g., sheep, goats, etc.); mammalian pets (e.g., dogs, cats, etc.); and rodents (e.g., mice, rats, etc.).

As used herein, the term “cardiomyopathy” refers to any disease or dysfunction that affects myocardium directly. The etiology of the disease or disorder may be, for example, inflammatory, metabolic, toxic, infiltrative, fibroplastic, hematological, genetic, or unknown in origin. Two fundamental forms are recognized (1) a primary type, consisting of heart muscle disease of unknown cause; and (2) a secondary type, consisting of myocardial disease of known cause or associated with a disease involving other organ systems. “Specific cardiomyopathy” refers to heart diseases associated with certain systemic or cardiac disorders; examples include hypertensive and metabolic cardiomyopathy. The cardiomyopathies include dilated cardiomyopathy (DCM), a disorder in which left and/or right ventricular systolic pump function is impaired, leading to progressive cardiac enlargement; hypertrophic cardiomyopathy, characterized by left ventricular hypertrophy without obvious causes such as hypertension or aortic stenosis; and restrictive cardiomyopathy, characterized by abnormal diastolic function and excessively rigid ventricular walls that impede ventricular filling. Cardiomyopathies also include left ventricular non-compaction, arrhythmogenic right ventricular cardiomyopathy, and arrhythmogenic right ventricular dysplasia.

“Heart failure” refers to the pathological state in which an abnormality of cardiac function is responsible for failure of the heart to pump blood at a rate commensurate with the requirements of the metabolizing tissues and/or allows the heart to do so only from an abnormally elevated diastolic volume. Heart failure includes systolic and diastolic failure. Patients with heart failure are classified into those with low cardiac output (typically secondary to ischemic heart disease, hypertension, dilated cardiomyopathy, and/or valvular or pericardial disease) and those with elevated cardiac output (typically due to hyperthyroidism, anemia, pregnancy, arteriovenous fistulas, beriberi, and Paget's disease). Heart failure includes heart failure with reduced ejection fraction (HFrEF) and heart failure with preserved ejection fraction (HFpEF).

The term “therapeutic gene” as used herein refers to a gene that, when expressed, confers a beneficial effect on the cell or tissue in which it is present, or on a mammal in which the gene is expressed. Examples of beneficial effects include amelioration of a sign or symptom of a condition or disease, prevention or inhibition of a condition or disease, or conferral of a desired characteristic. Therapeutic genes include genes that partially or wholly correct a genetic deficiency in a cell or mammal.

As used herein the term “cardiac cell” refers to any cell present in the heart that provides a cardiac function, such as heart contraction or blood supply, or otherwise serves to maintain the structure of the heart. Cardiac cells as used herein encompass cells that exist in the epicardium, myocardium or endocardium of the heart. Cardiac cells also include, for example, cardiac muscle cells or cardiomyocytes, and cells of the cardiac vasculatures, such as cells of a coronary artery or vein. Other non-limiting examples of cardiac cells include epithelial cells, endothelial cells, fibroblasts, cardiac stem or progenitor cells, cardiac conducting cells and cardiac pacemaking cells that constitute the cardiac muscle, blood vessels and cardiac cell supporting structure. Cardiac cells may be derived from stem cells, including, for example, embryonic stem cells or induced pluripotent stem cells.

### Expression Cassettes

### Expression Cassette Overview

The vectors of the disclosure may comprise any expression cassette described herein. In some aspects, the rAAV virions of the disclosure comprise a viral genome comprising an expression cassette as shown in FIG. 1, FIGS. 7A-7C, or variations thereof. The expression cassette may comprise a polynucleotide encoding any gene product described herein, or functional variant thereof, optionally operatively linked to a promoter, optionally an intron, optionally a polyadenylation (poly(A)) signal, optionally a woodchuck hepatitis virus post-transcriptional element (WPRE), and optionally a transcription termination signal. The expression cassette may be flanked by inverted terminal repeats (ITRs). These components provide the function of expressing the transgene after a host cell is targeted by, e.g., the rAAV virion. The promoter sequence, when present, controls expression of the polynucleotide encoding a gene product.

The expression cassette may comprise a polynucleotide encoding a DWORF polypeptide, or functional variant thereof, optionally operably linked to a promoter, optionally an intron, optionally a polyadenylation (poly(A)) signal, optionally a woodchuck hepatitis virus post-transcriptional element (WPRE), and optionally a transcription termination signal. The promoter sequence, when present, controls expression of the polynucleotide encoding the DWORF polypeptide, or functional variant thereof. The promoter sequence can be a cardiac cell-specific promoter. The promoter sequence can be further operably linked to an enhancer, such as any cardiac cell-specific enhancer described herein.

In any constructs shown in FIG. 1 and FIGS. 7A-7C, DWORF nucleotide sequence can be replaced by a nucleotide sequence encoding another gene product or polypeptide, such as any gene product or polypeptide described herein (e.g., see the description of transgenes and gene products encoded by such transgenes below). Accordingly, in some embodiments, provided herein is any expression cassette shown in FIG. 1 and FIGS. 7A-7C wherein the DWORF nucleotide sequence is replaced by a nucleotide sequence encoding another gene product or polypeptide.

Also, in any expression cassettes shown in Table 1, DWORF nucleotide sequence can be replaced by a nucleotide sequence encoding another gene product or polypeptide, such as any gene product or polypeptide described herein (e.g., see the description of transgenes and gene products encoded by such transgenes below). Further, in any expression cassette shown in Table 1, the ITR sequences can be omitted. Accordingly, in some embodiments, provided herein is any expression cassette shown in Table 1 wherein the DWORF nucleotide sequence is replaced by a nucleotide sequence encoding another gene product or polypeptide, and/or wherein the specified ITR sequence is not present.

### Transgenes

In some embodiments, the expression cassette of the disclosure comprises a transgene. Transgenes can include nucleotide sequences encoding any polypeptide for use in treating or preventing a heart disease or disorder, or alleviating symptoms therefrom. The promoters, enhancers and combinations thereof described herein are operably linked to a transgene encoding a product. A transgene can be a gene or nucleotide sequence that encodes a product, or functional fragment thereof. A product can be, for example, a polypeptide or a non-coding nucleotide. By non-coding nucleotide, it is meant that the sequence transcribed from the transgene or nucleotide sequence is not translated into a polypeptide. In some embodiments, the product encoded by the transgene or nucleotide operably linked to an enhancer described herein is a non-coding polynucleotide. A non-coding polynucleotide can be an RNA, such as for example a microRNA (miRNA or mIR), short hairpin RNA (shRNA), long non-coding RNA (lnRNA), and/or a short interfering RNA (siRNA). In some embodiments, the transgene encodes a product natively expressed by a cardiac cell, e.g., a cardiomyocyte. In some embodiments, the transgene encodes a product natively expressed in a cell type other than a cardiac cell. Without limitation, cell types other than cardiac fibroblasts can be from any multicellular organism, single-celled organism, or microorganism.

In some embodiments, the transgene encodes a polypeptide. In some embodiments, the transgene encodes a non-coding polynucleotide such as, for example, a microRNA (miRNA or mIR).

In some embodiments, the transgene comprises a sequence encoding a product selected from cadherins, connexins, Cx43, growth factors such as fibroblast growth factor (FGF)-2 and transforming growth factor-β, cytokines such as interleukin (IL)-1β and the IL-6 family, leukemia inhibitory factor, cardiotrophin-1, cardiogenic transcription factors, insulin-like growth factor, GATA4, MEF2C, TBX5, ESRRG, MESP1, MYOCD, ZFPM2, HAND2, miR-1, miR-133, Oct4, Sox2, Klf4, c-Myc, SRF, SMARCD3, Nkx2-5, Akt, PKB, Baf60c, BMP4, miR-208, and miR-499.

In some embodiments, the transgene encodes a functional cardiac protein. In some embodiments, the gene product is a genome-editing endonuclease (optionally with a guide RNA, single-guide RNA, and/or repair template) that replaces or repairs a non-functional cardiac protein into a functional cardiac protein. Functional cardiac proteins include, but are not limited to cardiac troponin T; a cardiac sarcomeric protein; β-myosin heavy chain; myosin ventricular essential light chain 1; myosin ventricular regulatory light chain 2; cardiac a-actin; a-tropomyosin; cardiac troponin I; cardiac myosin binding protein C; four-and-a-half LIM protein 1; titin; 5′-AMP-activated protein kinase subunit gamma-2; troponin I type 3, myosin light chain 2, actin alpha cardiac muscle 1; cardiac LIM protein; caveolin 3 (CAV3); galactosidase alpha (GLA); lysosomal-associated membrane protein 2 (LAMP2); mitochondrial transfer RNA glycine (MTTG); mitochondrial transfer RNA isoleucine (MTTI); mitochondrial transfer RNA lysine (MTTK); mitochondrial transfer RNA glutamine (MTTQ); myosin light chain 3 (MYL3); troponin C (TNNC1); transthyretin (TTR); sarcoendoplasmic reticulum calcium-ATPase 2a (SERCA2a); stromal-derived factor-1 (SDF-1); adenylate cyclase-6 (AC6); beta-ARKct (0-adrenergic receptor kinase C terminus); fibroblast growth factor (FGF); platelet-derived growth factor (PDGF); vascular endothelial growth factor (VEGF); hepatocyte growth factor; hypoxia inducible growth factor; thymosin beta 4 (TMSB4X); nitric oxide synthase-3 (NOS3); unocartin 3 (UCN3); melusin; apoplipoprotein-E (ApoE); superoxide dismutase (SOD); and S100A1 (a small calcium binding protein; see, e.g., Ritterhoff and Most (2012) Gene Ther. 19:613; Kraus et al. (2009) Mol. Cell. Cardiol. 47:445).

In some embodiments, the transgene can treat or prevent coronary heart disease. In some embodiments, the transgene comprises a sequence encoding a product selected from vascular endothelial growth factor (VEGF), a VEGF isoform, VEGF-A, VEGF-B, VEGF-C, VEGF-D, VEGF-D^{dNdC}, VEGF-A₁₁₆A, VEGF-A₁₆₅, VEGF-A₁₂₁, VEGF-2, placenta growth factor (PIGF), fibroblast growth factor 4 (FGF-4), human growth factor (HGF), human granulocyte colony-stimulating factor (hGCSF), and hypoxia inducible factor 1α (HIF-1α).

In some embodiments, the transgene can treat or prevent heart failure. In some embodiments, the transgene can treat or prevent chronic heart failure. In some embodiments, the transgene comprises a sequence encoding a product selected from SERCA2a, stromal cell-derived factor-1 (SDF-1), adenylyl cyclase type 6, S100A1, miRNA-17-92, miR-302-367, anti-miR-29a, anti-miR-30a, antimiR-141, cyclin A2, cyclin-dependent kinase 2, Tbx20, miRNA-590, miRNA-199, anti-sense oligonucleotide against Lp(a), interfering RNA against PCSK9, anti-sense oligonucleotide against apolipoprotein C-III, lipoprotein lipases^{S447X}, anti-sense oligonucleotide against apolipoprotein B, anti-sense oligonucleotide against c-myc, and E2F oligonucleotide decoy.

In some embodiments, the transgene encodes a gene product whose expression complements a defect in a gene responsible for a genetic disorder. The disclosure polynucleotides encoding one or more of the following—e.g., for use, without limitation, in the disorder indicated in parentheses, or for other disorders caused by each: TAZ (Barth syndrome); FXN (Freidrich's Ataxia); CASQ2 (CPVT); FBN1 (Marfan); RAF1 and SOS1s (Noonan); SCN5A (Brugada); KCNQ1 and KCNH2s (Long QT Syndrome); DMPK (Myotonic Dystrophy 1); LMNA (Limb Girdle Dystrophy Type 1B); JUP (Naxos); TGFBR2 (Loeys-Dietz); EMD (X-Linked EDMD); and ELN (SV Aortic Stenosis). In some embodiments, a polynucleotide encodes one or more of: cardiac troponin T (TNNT2); BAG family molecular chaperone regulator 3 (BAG3); myosin heavy chain (MYH7); tropomyosin 1 (TPM1); myosin binding protein C (MYBPC3); 5′-AMP-activated protein kinase subunit gamma-2 (PRKAG2); troponin I type 3 (TNNI3); titin (TTN); myosin, light chain 2 (MYL2); actin, alpha cardiac muscle 1 (ACTC1); potassium voltage-gated channel, KQT-like subfamily, member 1 (KCNQ1); myocyte enhancer factor 2c (MEF2C); and cardiac LIM protein (CSRP3).

In some embodiments, the transgene comprises a nucleotide sequence encoding a protein selected from DWORF, junctophilin (e.g., JPH2), BAG family molecular chaperone regulator 3 (BAG3), phospholamban (PLN), alpha-crystallin B chain (CRYAB), LMNA (such as Lamin A and Lamin C isoforms), troponin I type 3 (TNNI3), lysosomal-associated membrane protein 2 (LAMP2, such as LAMP2a, LAMP2b and LAMP2c isoforms), desmoplakin (DSP, such as DPI and DPII isoforms), desmoglein 2 (DSG2), and junction plakoglobin (JUP). In some embodiments, the transgene comprises a nucleotide sequence encoding a human protein. In some embodiments, the transgene comprises a human nucleotide sequence (a human DNA sequence). In some embodiments, the transgene comprises a DNA sequence that has been codon-optimized. In some embodiments, the transgene comprises a nucleotide sequence encoding a wild-type protein, or a functionally active fragment thereof.

In some embodiments, the transgene comprises a polynucleotide sequence encoding a DWORF polypeptide.

In some embodiments, the transgene comprises a polynucleotide sequence encoding a junctophilin 2 (JPH2) polypeptide. In some embodiments, the transgene comprises a polynucleotide sequence encoding a full-length JPH2 polypeptide. In some embodiments, the transgene comprises a polynucleotide sequence encoding an N-terminal fragment of the JPH2 polypeptide. In some embodiments, the transgene comprises a polynucleotide sequence encoding an N-terminal fragment of the JPH2 polypeptide, which retains the JPH2 activity.

In some embodiments, the transgene comprises a polynucleotide sequence encoding a BAG3 polypeptide.

In some embodiments, the transgene comprises a polynucleotide sequence encoding a CRYAB polypeptide.

In some embodiments, the transgene comprises a polynucleotide sequence encoding a LMNA polypeptide. In some embodiments, the transgene comprises a polynucleotide sequence encoding the LaminA isoform of LMNA. In some embodiments, the transgene comprises a polynucleotide sequence encoding the LaminC isoform of LMNA.

In some embodiments, the transgene comprises a polynucleotide sequence encoding a TNNI3 polypeptide.

In some embodiments, the transgene comprises a polynucleotide sequence encoding a PLN polypeptide.

In some embodiments, the transgene comprises a polynucleotide sequence encoding a LAMP2 polypeptide. In some embodiments, the transgene comprises a polynucleotide sequence encoding the LAMP2a isoform. In some embodiments, the transgene comprises a polynucleotide sequence encoding the LAMP2b isoform. In some embodiments, the transgene comprises a polynucleotide sequence encoding the LAMP2c isoform.

In some embodiments, the transgene comprises a polynucleotide sequence encoding a DSP polypeptide. In some embodiments, the transgene comprises a polynucleotide sequence encoding the DPI isoform of DSP. In some embodiments, the transgene comprises a polynucleotide sequence encoding the DPII isoform of DSP.

In some embodiments, the transgene comprises a polynucleotide sequence encoding a DSG2 polypeptide.

In some embodiments, the transgene comprises a polynucleotide sequence encoding a JUP polypeptide.

It is appreciated that the transgenes described herein are non-limiting and transgenes useful for treating a heart disease may be discovered for use in the expression cassettes described herein.

### DWORF Transgene

In some embodiments, the expression cassette of the present disclosure comprises a polynucleotide sequence encoding a DWORF polypeptide. In some embodiments, the expression cassette provides increased expression of a DWORF polypeptide in cardiac cell. In some embodiments, the cardiac cell is a cardiomyocyte. In some embodiments, expression of the DWORF polypeptide may be increased 5%, 10%, 15%, 20%, or 25% compared to expression of the DWORF polypeptide factor in an untreated subject. In some embodiments, expression of the DWORF polypeptide may be increased 1-fold, 2-fold, 3-fold, 4-fold, or 5-fold compared to expression of the DWORF polypeptide in an untreated subject. In some embodiments, the DWORF polypeptide may be expressed at any detectable level in the cardiac cell, whereas the DWORF polypeptide may not be expressed, or expressed at undetectable levels, in an untreated subject. Put another way, the cardiac cell to which the rAAV virion is administered may express a DWORF polypeptide in higher abundance than in a cardiac cell that has only endogenous (i.e., native) expression of the DWORF polypeptide.

DWORF polypeptide is an endogenous enhancer of SERCA calcium pump activity, a desirable drug target for regulation of cardiac contractility. DWORF is also an unusually small protein, which makes it a good candidate for delivery to a target cell or tissue by rAAV virions. Because DWORF is an endogenous protein, expression of DWORF in humans would not be immunogenic, allowing for long-term dosing and expression. The structural features of DWORF polypeptides are as follows. First, the polypeptides may have 5 to 35 consecutive residues of the Dwarf Open Reading Frame (DWORF), located on chromosome 3 of a mammalian species, including mouse and human (Nelson et al. Science. 351: 271-275 (2016); U.S. Pat. No. 10,570,183). Thus, the term “a peptide having no more than X consecutive residues,” even when including the term “comprising,” cannot be understood to comprise a greater number of consecutive residues. In general, the peptides will be 35 residues or less, again, comprising no more than 20 consecutive residues of DWORF. The overall length may be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 residues. Ranges of peptide length of 5-34/35 residues, 6-34/35 residues, 7-50 residues, 7-25, residues, 5-20 residues, 6-20 residues, 7-20 residues, and 7-15 residues are contemplated. The number of consecutive DWORF residues may be 5, 6, 7, 8, 9, 10, 11, 12, 13, 14, 15, 16, 17, 18, 19 or 20. Ranges of consecutive residues of 5-20 residues, 5-20 residues, 6-20 residues, 7-20 residues and 5-15 residues, 5-15, residues, 6-15 residues or 7-15 residues are contemplated. Illustrative DWORF sequences can be found in Table 2a.

In some embodiments, DWORF polypeptide is human DWORF polypeptide. In some embodiments, the expression cassette comprises a single polynucleotide sequence encoding a dwarf open reading frame (DWORF) polypeptide. In some embodiments, the polynucleotide sequence encoding DWORF is codon optimized. In some embodiments, the DWORF polypeptide comprises a polypeptide sequence that shares at least 95% identity to SEQ ID NO: 1, SEQ ID NO: 3, SEQ ID NO: 5, SEQ ID NO: 7, or SEQ ID NO: 9. In some embodiments, the DWORF polypeptide comprises a polypeptide sequence that shares at least 98% identity to SEQ ID NO: 1, SEQ ID NO: 3, SEQ ID NO: 5, SEQ ID NO: 7, or SEQ ID NO: 9. In some embodiments, the DWORF polypeptide comprises the polypeptide sequence of SEQ ID NO: 1, SEQ ID NO: 3, SEQ ID NO: 5, SEQ ID NO: 7, or SEQ ID NO: 9.

TABLE 2a
Illustrative DWORF Sequences

| DWORF | | |
|---|---|---|
| Variant | DWORF Polypeptide | Nucleotide (Open Reading Frame) |
| Mouse | MAEKESTSPHLMVPILLLVGWIVGCIIVI | ATGGCTGAGAAAGAGTCAACATCACCACACCTCATGGTTCC |
| Variant | YIVFF (SEQ ID NO: 1) | CATTCTTCTCCTGGTTGGATGGATTGTAGGCTGCATCATCG |
| | | TTATTTACATTGTCTTCTTCTAA (SEQ ID NO: 2) |
| Human | MAEKAGSTFSHLLVPILLLIGWIVGCIIM | ATGGCTGAAAAAGCGGGGTCTACATTTTCACACCTTCTGGT |
| Variant | IYVVFS (SEQ ID NO: 3) | TCCTATTCTTCTCCTGATTGGCTGGATTGTGGGCTGCATCA |
| | | TAATGATTTATGTTGTCTTCTCTTAG (SEQ ID NO: 4) |
| Artificial | MAEKAESTSPHLMVPILLLVGWIVGCIIV | ATGGCTGAGAAAGCAGAGTCAACATCACCACACCTCATGGT |
| | IYIVFF (SEQ ID NO: 5) | TCCCATTCTTCTCCTGGTTGGATGGATTGTAGGCTGCATCA |
| | | TCGTTATTTACATTGTCTTCTTCTAA (SEQ ID NO: 6) |
| Artificial | MAEKESTSPHLIVPILLLVGWIVGCIIVI | ATGGCTGAGAAAGAGTCAACATCACCACACCTCATTGTTCC |
| | YIVFF (SEQ ID NO: 7) | CATTCTTCTCCTGGTTGGATGGATTGTAGGCTGCATCATCG |
| | | TTATTTACATTGTCTTCTTCTAA (SEQ ID NO: 8) |
| Artificial | MAEKAESTSPHLIVPILLLVGWIVGCIIV | ATGGCTGAGAAAGCAGAGTCAACATCACCACACCTCATTGT |
| | IYIVFF (SEQ ID NO: 9) | TCCCATTCTTCTCCTGGTTGGATGGATTGTAGGCTGCATCA |
| | | TCGTTATTTACATTGTCTTCTTCTAA (SEQ ID NO: 10) |
| Human | MAEKESTSPHLMVPILLLVGWIVGCIIVI | ATGGCAGAGAAGGCTGGAAGCACTTTCTCTCACCTGCTCGT |
| Variant | YIVFF (SEQ ID NO: 32) | GCCGATTTTGCTTTTGATTGGGTGGATAGTTGGCTGTATCA |
| | | TAATGATCTACGTTGTCTTTTCATAG (SEQ ID NO: 33) |
| Human | MAEKGSTFSHLLVPILLLIGWIVGCIIMI | ATGGCCGAGAAGGGCAGCACCTTCAGCCACCTGCTGGTGCC |
| Variant | YVVFS (SEQ ID NO: 43) | CATCCTGCTGCTGATCGGCTGGATCGTGGGCTGCATCATCA |
| | | TGATCTACGTGGTGTTCAGC (SEQ ID NO: 44) |
| Codon | MAEKESTSPHLMVPILLLVGWIVGCIIVI | ATGGCCGAGAAGGAATCTACCAGCCCCCACCTGATGGTGCC |
| Optimized | YIVFF (SEQ ID NO: 1) | TATTCTGCTGCTGGTGGGCTGGATCGTCGGCTGCATCATCG |
| Mouse DWORF | | TGATCTACATCGTGTTCTTCTGA (SEQ ID NO: 76) |
| Codon | MAEKAGSTFSHLLVPILLLIGWIVGCIIM | ATGGCCGAGAAGGCCGGATCTACCTTCAGCCACCTGCTGGT |
| Optimized | IYVVFS (SEQ ID NO: 3) | CCCTATTCTGCTGCTGATCGGCTGGATCGTGGGCTGCATCA |
| Human DWORF | | TCATGATCTACGTGGTGTTCAGCTGA (SEQ ID NO: 77) |

### Other Exemplary Transgenes

In some embodiments, the expression cassette of the present disclosure comprises a polynucleotide sequence encoding another gene product (not DWORF), for example, a polypeptide selected from JPH2, BAG3, CRYAB, LMNA (e.g., Lamin A or Lamin C isoform), TNNI3, PLN, LAMP2 (e.g., LAMP2a, LAMP2b or LAMP2c isoform), DSP (e.g., DPI or DPII isoform), desmoglein 2 (DSG2), and junction plakoglobin (JUP). In some embodiments, the expression cassette provides increased expression of the gene product in a cardiac cell. In some embodiments, the cardiac cell is a cardiomyocyte. In some embodiments, expression of the polypeptide encoded by the polynucleotide sequence may be increased 5%, 10%, 15%, 20%, or 25% compared to expression in an untreated subject. In some embodiments, expression of the polypeptide encoded by the polynucleotide sequence may be increased 1-fold, 2-fold, 3-fold, 4-fold, or 5-fold compared to expression in an untreated subject. In some embodiments, the polypeptide encoded by the polynucleotide sequence may be expressed at any detectable level in the cardiac cell, whereas it may not be expressed, or expressed at undetectable levels, in an untreated subject. In some embodiments, the cardiac cell to which a vector described herein is administered may express a polypeptide encoded by the polynucleotide sequence in higher abundance than in a cardiac cell that has only endogenous (i.e., native) expression of the polypeptide.

In some embodiments, the polypeptide is a human polypeptide. In some embodiments, the polynucleotide sequence encoding the polypeptide is codon optimized. In some embodiments, the expression cassette comprises a single polynucleotide sequence encoding a polypeptide. In some embodiments, the expression cassette comprises two polynucleotide sequences encoding a polypeptide. In some embodiments, the expression cassette comprises two polynucleotide sequences encoding a polypeptide, wherein at least one of the sequences is codon-optimized.

In some embodiments, a polynucleotide sequence encodes JPH2, e.g., human JPH2. In some embodiments, a polynucleotide sequence has at least 75%, 80%, 85%, 90%, 95%, 98%, 99% or 100% sequence identity to SEQ ID NO:201. In some embodiments, a polynucleotide sequence is a codon-optimized sequence encoding JPH2, e.g., human JPH2. In some embodiments, the gene product or polypeptide expressed using any expression construct described herein is JPH2, e.g., human JPH2. In some embodiments, the JPH2 polypeptide has at least 75%, 80%, 85%, 90%, 95%, 98%, 99% or 100% sequence identity to SEQ ID NO:202.

In some embodiments, a polynucleotide sequence encodes an N-terminal fragment of JPH2, e.g., human JPH2. In some embodiments, a polynucleotide sequence of an N-terminal fragment of JPH2 has at least 75%, 80%, 85%, 90%, 95%, 98%, 99% or 100% sequence identity to SEQ ID NO:227. In some embodiments, a polynucleotide sequence is a codon-optimized sequence encoding an N-terminal fragment of JPH2, e.g., human JPH2. In some embodiments, the gene product or polypeptide expressed using any expression construct described herein is an N-terminal fragment of JPH2, e.g., human JPH2. In some embodiments, the N-terminal fragment of JPH2 polypeptide has at least 75%, 80%, 85%, 90%, 95%, 98%, 99% or 100% sequence identity to SEQ ID NO:228. The human sequences of an N-terminal fragment of JPH2 correspond to the mouse JPH2 N-terminal peptide with amino acids 1-565, generated by a Calpain cleavage (see Guo et al., 2018, Science 362, doi: 10.1126/science.aan3303).

In some embodiments, a polynucleotide sequence encodes BAG3, e.g., human BAG3. In some embodiments, a polynucleotide sequence has at least 75%, 80%, 85%, 90%, 95%, 98%, 99% or 100% sequence identity to SEQ ID NO:203. In some embodiments, a polynucleotide sequence is a codon-optimized sequence encoding BAG3, e.g., human BAG3. In some embodiments, the gene product or polypeptide expressed using any expression construct described herein is BAG3, e.g., human BAG3. In some embodiments, the BAG3 polypeptide has at least 75%, 80%, 85%, 90%, 95%, 98%, 99% or 100% sequence identity to SEQ ID NO:204.

In some embodiments, a polynucleotide sequence encodes CRYAB, e.g., human CRYAB. In some embodiments, a polynucleotide sequence has at least 75%, 80%, 85%, 90%, 95%, 98%, 99% or 100% sequence identity to SEQ ID NO:205. In some embodiments, a polynucleotide sequence is a codon-optimized sequence encoding CRYAB, e.g., human CRYAB. In some embodiments, the gene product or polypeptide expressed using any expression construct described herein is CRYAB, e.g., human CRYAB. In some embodiments, the CRYAB polypeptide has at least 75%, 80%, 85%, 90%, 95%, 98%, 99% or 100% sequence identity to SEQ ID NO:206.

In some embodiments, a polynucleotide sequence encodes LMNA, e.g., human LMNA. In some embodiments, a polynucleotide sequence encodes Lamin A isoform of LMNA, e.g., human Lamin A. In some embodiments, a polynucleotide sequence has at least 75%, 80%, 85%, 90%, 95%, 98%, 99% or 100% sequence identity to SEQ ID NO:207. In some embodiments, a polynucleotide sequence encodes Lamin C isoform of LMNA, e.g., human Lamin C. In some embodiments, a polynucleotide sequence has at least 75%, 80%, 85%, 90%, 95%, 98%, 99% or 100% sequence identity to SEQ ID NO:209. In some embodiments, a polynucleotide sequence is a codon-optimized sequence encoding an LMNA polypeptide. In some embodiments, a polynucleotide sequence is a codon-optimized sequence encoding Lamin A or Lamin C, e.g., human Lamin A or Lamin C. In some embodiments, the gene product or polypeptide expressed using any expression construct described herein is LMNA, e.g., human LMNA. In some embodiments, the gene product or polypeptide expressed using any expression construct described herein is Lamin A isoform of LMNA, e.g., human. In some embodiments, the Lamin A polypeptide has at least 75%, 80%, 85%, 90%, 95%, 98%, 99% or 100% sequence identity to SEQ ID NO:208. In some embodiments, the gene product or polypeptide expressed using any expression construct described herein is Lamin C isoform of LMNA, e.g., human. In some embodiments, the Lamin C polypeptide has at least 75%, 80%, 85%, 90%, 95%, 98%, 99% or 100% sequence identity to SEQ ID NO:210.

In some embodiments, a polynucleotide sequence encodes TNNI3, e.g., human TNNI3. In some embodiments, a polynucleotide sequence has at least 75%, 80%, 85%, 90%, 95%, 98%, 99% or 100% sequence identity to SEQ ID NO:211. In some embodiments, a polynucleotide sequence is a codon-optimized sequence encoding TNNI3, e.g., human TNNI3. In some embodiments, the gene product or polypeptide expressed using any expression construct described herein is TNNI3, e.g., human TNNI3. In some embodiments, the TNNI3 polypeptide has at least 75%, 80%, 85%, 90%, 95%, 98%, 99% or 100% sequence identity to SEQ ID NO:212.

In some embodiments, a polynucleotide sequence encodes PLN, e.g., human PLN. In some embodiments, a polynucleotide sequence has at least 75%, 80%, 85%, 90%, 95%, 98%, 99% or 100% sequence identity to SEQ ID NO:229. In some embodiments, a polynucleotide sequence is a codon-optimized sequence encoding PLN, e.g., human PLN. In some embodiments, the gene product or polypeptide expressed using any expression construct described herein is PLN, e.g., human PLN. In some embodiments, the PLN polypeptide has at least 75%, 80%, 85%, 90%, 95%, 98%, 99% or 100% sequence identity to SEQ ID NO:230.

In some embodiments, a polynucleotide sequence encodes LAMP2, e.g., human LAMP2. In some embodiments, a polynucleotide sequence encodes LAMP2a isoform of LAMP2, e.g., human LAMP2a. In some embodiments, a polynucleotide sequence has at least 75%, 80%, 85%, 90%, 95%, 98%, 99% or 100% sequence identity to SEQ ID NO:213. In some embodiments, a polynucleotide sequence encodes LAMP2b isoform of LAMP, e.g., human LAMP2b. In some embodiments, a polynucleotide sequence has at least 75%, 80%, 85%, 90%, 95%, 98%, 99% or 100% sequence identity to SEQ ID NO:215. In some embodiments, a polynucleotide sequence encodes LAMP2c isoform of LAMP, e.g., human LAMP2c. In some embodiments, a polynucleotide sequence has at least 75%, 80%, 85%, 90%, 95%, 98%, 99% or 100% sequence identity to SEQ ID NO:217. In some embodiments, a polynucleotide sequence is a codon-optimized sequence encoding LAMP2, e.g., human LAMP2. In some embodiments, a polynucleotide sequence is a codon-optimized sequence encoding LAMP2a, LAMP2b or LAMP2c. In some embodiments, the gene product or polypeptide expressed using any expression construct described herein is a LAMP2 polypeptide, e.g., human LAMP2. In some embodiments, the gene product or polypeptide expressed using any expression construct described herein is LAMP2a isoform of LAMP2, e.g., human. In some embodiments, the LAMP2a polypeptide has at least 75%, 80%, 85%, 90%, 95%, 98%, 99% or 100% sequence identity to SEQ ID NO:214. In some embodiments, the gene product or polypeptide expressed using any expression construct described herein is LAMP2b isoform of LAMP2, e.g., human. In some embodiments, the LAMP2b polypeptide has at least 75%, 80%, 85%, 90%, 95%, 98%, 99% or 100% sequence identity to SEQ ID NO:216. In some embodiments, the gene product or polypeptide expressed using any expression construct described herein is LAMP2c isoform of LAMP2, e.g., human. In some embodiments, the LAMP2c polypeptide has at least 75%, 80%, 85%, 90%, 95%, 98%, 99% or 100% sequence identity to SEQ ID NO:218.

In some embodiments, a polynucleotide sequence encodes DSP, e.g., human DSP. In some embodiments, a polynucleotide sequence encodes DPI isoform of DSP, e.g., human DPI. In some embodiments, a polynucleotide sequence has at least 75%, 80%, 85%, 90%, 95%, 98%, 99% or 100% sequence identity to SEQ ID NO:219. In some embodiments, a polynucleotide sequence encodes DPII isoform of DSP, e.g., human DPII. In some embodiments, a polynucleotide sequence has at least 75%, 80%, 85%, 90%, 95%, 98%, 99% or 100% sequence identity to SEQ ID NO:221. In some embodiments, a polynucleotide sequence is a codon-optimized sequence encoding a DSP polypeptide. In some embodiments, a polynucleotide sequence is a codon-optimized sequence encoding DPI or DPII, e.g., human DPI or DPII. In some embodiments, the gene product or polypeptide expressed using any expression construct described herein is DSP, e.g., human DSP. In some embodiments, the gene product or polypeptide expressed using any expression construct described herein is DPI isoform of DSP, e.g., human. In some embodiments, the DPI polypeptide has at least 75%, 80%, 85%, 90%, 95%, 98%, 99% or 100% sequence identity to SEQ ID NO:220. In some embodiments, the gene product or polypeptide expressed using any expression construct described herein is DPII isoform of DSP, e.g., human. In some embodiments, the DPII polypeptide has at least 75%, 80%, 85%, 90%, 95%, 98%, 99% or 100% sequence identity to SEQ ID NO:222.

In some embodiments, a polynucleotide sequence encodes DSG2, e.g., human DSG2. In some embodiments, a polynucleotide sequence has at least 75%, 80%, 85%, 90%, 95%, 98%, 99% or 100% sequence identity to SEQ ID NO:223. In some embodiments, a polynucleotide sequence is a codon-optimized sequence encoding DSG2, e.g., human DSG2. In some embodiments, the gene product or polypeptide expressed using any expression construct described herein is DSG2, e.g., human DSG2. In some embodiments, the DSG2 polypeptide has at least 75%, 80%, 85%, 90%, 95%, 98%, 99% or 100% sequence identity to SEQ ID NO:224.

In some embodiments, a polynucleotide sequence encodes JUP, e.g., human JUP. In some embodiments, a polynucleotide sequence has at least 75%, 80%, 85%, 90%, 95%, 98%, 99% or 100% sequence identity to SEQ ID NO:225. In some embodiments, a polynucleotide sequence is a codon-optimized sequence encoding JUP, e.g., human JUP. In some embodiments, the gene product or polypeptide expressed using any expression construct described herein is JUP, e.g., human JUP. In some embodiments, the JUP polypeptide has at least 75%, 80%, 85%, 90%, 95%, 98%, 99% or 100% sequence identity to SEQ ID NO:226.

In some embodiments, any other polynucleotide sequence described herein can be used in any expression construct described herein. In some embodiments, such polynucleotide sequence encodes any gene product or polypeptide described herein. The polynucleotide sequence can be sequence-optimized (such as for expression in a human). In some embodiments, the sequence encodes a human polypeptide. The sequences of the polynucleotides and polypeptides described herein are known in the art. Sequences that have at least 75%, 80%, 85%, 90%, 95%, 98%, 99% or 100% sequence identity to such sequence are also contemplated herein. Illustrative sequences can be found in Table 2b.

TABLE 2b
Illustrative Gene product Sequences

| Transgene | Polypeptide | Nucleotide (Open Reading Frame) |
|---|---|---|
| Human | MSGGRFDFDDGGAYCGGWEG | ATGAGTGGGGGCCGCTTCGACTTTGATGATG |
| JPH2 | GKAHGHGLCTGPKGQGEYSGS | GAGGGGCGTACTGCGGGGGCTGGGAGGGG |
| (the N- | WNFGFEVAGVYTWPSGNTFEG | GGAAAGGCCCATGGGCATGGACTGTGCACA |
| terminal | YWSQGKRHGLGIETKGRWLYK | GGCCCCAAGGGCCAGGGCGAATACTCTGGC |
| part of the | GEWTHGFKGRYGIRQSSSSGAK | TCCTGGAACTTTGGCTTTGAGGTGGCAGGTG |
| sequence | YEGTWNNGLQDGYGTETYADG | TCTACACCTGGCCCAGCGGAAACACCTTTGA |
| that, in | GTYQGQFTNGMRHGYGVRQSV | GGGATACTGGAGCCAGGGCAAACGGCATGG |
| some | PYGMAVVVRSPLRTSLSSLRSE | GCTGGGCATAGAGACCAAGGGGCGCTGGCT |
| instances, | HSNGTVAPDSPASPASDGPALPS | CTACAAGGGCGAGTGGACACATGGCTTCAA |
| can be used | PAIPRGGFALSLLANAEAAARAP | GGGACGCTACGGAATCCGGCAGAGCTCAAG |
| on its own, | KGGGLFQRGALLGKLRRAESR | CAGCGGTGCCAAGTATGAGGGCACCTGGAA |
| as an | TSVGSQRSRVSFLKSDLSSGASD | CAATGGCCTGCAAGACGGCTATGGCACCGA |
| alternative | AASTASLGEAAEGADEAAPFEA | GACCTATGCTGATGGAGGGACGTACCAAGG |
| to the full- | DIDATTTETYMGEWKNDKRSG | CCAGTTCACCAACGGCATGCGCCATGGCTAC |
| length | FGVSERSSGLRYEGEWLDNLRH | GGAGTACGCCAGAGCGTGCCCTACGGGATG |
| JPH2, is | GYGCTTLPDGHREEGKYRHNV | GCCGTGGTGGTGCGCTCGCCGCTGCGCACG |
| shown in | LVKDTKRRMLQLKSNKVRQKV | TCGCTGTCGTCCCTGCGCAGCGAGCACAGC |
| bold) | EHSVEGAQRAAAIARQKAEIAA | AACGGCACGGTGGCCCCGGACTCTCCCGCC |
| | SRTSHAKAKAEAAEQAALAAN | TCGCCGGCCTCCGACGGCCCCGCGCTGCCC |
| | QESNIARTLARELAPDFYQPGPE | TCGCCCGCCATCCCGCGTGGCGGCTTCGCG |
| | YQKRRLLQEILENSESLLEPPDR | CTCAGCCTCCTGGCCAATGCCGAGGCGGCC |
| | GAGAAGLPQPPRESPQLHERET | GCGCGGGCGCCCAAGGGCGGCGGCCTCTTC |
| | PRPEGGSPSPAGTPPQPKRPRPG | CAGCGGGGCGCGCTGCTGGGCAAGCTGCGG |
| | VSKDGLLSPGAWNGEPSGEGSR | CGCGCAG |
| | SVTPSEGAGRRSPARPATERMAI | AGTCGCGCACGTCCGTGGGTAGCCAGCGCA |
| | EALQAPPAPSREPEVALYQGYH | GCCGTGTCAGCTTCCTTAAGAGCGACCTCAG |
| | SYAVRTTPPEPPPFEDQPEPEVSGS | CTCGGGCGCCAGCGACGCCGCGTCCACCGC |
| | ESAPSSPATAPLQAPTLRGPEPAR | CAGCCTGGGAGAGGCCGCCGAGGGCGCCGA |
| | ETPAKLEPKPIIPKAEPRAKARKTE | CGAGGCCGCACCCTTCGAGGCCGATATCGA |
| | ARGLTKAGAKKKARKEAALAAE | CGCCACCACCACCGAGACCTACATGGGCGA |
| | AEVEVEEVPNTILICMVILLNIGLA | GTGGAAGAACGACAAACGCTCGGGCTTCGG |
| | ILFVHLLT (SEQ ID NO: 202) | CGTGAGCGAACGCTCCAGTGGCCTCCGCTA |
| | | CGAGGGCGAGTGGCTGGACAACCTGCGCCA |
| | | CGGCTATGGCTGCACCACGCTGCCCGACGG |
| | | CCACCGCGAGGAGGGCAAGTACCGCCACAA |
| | | CGTGCTGGTCAAGGACACCAAGCGCCGCAT |
| | | GCTGCAGCTCAAGAGCAACAAGGTCCGCCA |
| | | GAAAGTGGAGCACAGTGTGGAGGGTGCCCA |
| | | GCGCGCCGCTGCTATCGCGCGCCAGAAGGC |
| | | CGAGATTGCCGCCTCCAGGACAAGCCACGC |
| | | CAAGGCCAAAGCTGAGGCAGCGGAACAGGC |
| | | CGCCCTGGCTGCCAACCAGGAGTCCAACATT |
| | | GCTCGCACTTTGGCCAGGGAGCTGGCTCCG |
| | | GACTTCTACCAGCCAGGTCCGGAATATCAGA |
| | | AGCGCCGGCTGCTGCAGGAGATCCTGGAGA |
| | | ACTCGGAGAGCCTGCTGGAGCCCCCCGACC |
| | | GGGGCGCCGGCGCAGCGGGCCTCCCACAGC |
| | | CGCCCCGCGAGAGCCCGCAGCTGCACGAGC |
| | | GTGAGACCCCTCGGCCCGAGGGTGGCTCCC |
| | | CGTCACCGGCCGGGACGCCCCCGCAGCCCA |
| | | AGCGGCCCAGGCCCGGGGTGTCCAAGGACG |
| | | GCCTGCTGAGCCCAGGCGCCTGGAACGGCG |
| | | AGCCCAGCGGTGAGGGCAGCCGGTCAGTCA |
| | | CTCCGTCCGAGGGCGCGGGCCGCCGCAGCC |
| | | CCGCGCGTCCAGCCACCGAGCGCATGGCCA |
| | | TCGAGGCTCTGCAGGCACCGCCTGCGCCGT |
| | | CGCGGGAGCCGGAGGTGGCGCTTTACCAGG |
| | | GCTACCACAGCTATGCTGTGCGCACCACGCC |
| | | GCCCGAGCCCCCACCCTTTGAGGACCAGCCCG |
| | | AGCCCGAGGTCTCCGGGTCCGAGTCCGCGCCCT |
| | | CGTCCCCGGCCACCGCCCCGCTGCAGGCCCCCA |
| | | CGCTCCGAGGCCCCGAGCCTGCACGCGAGACC |
| | | CCCGCCAAGCTGGAGCCCAAGCCCATCATCCCC |
| | | AAAGCCGAGCCCAGGGCCAAGGCCCGCAAGAC |
| | | TGAGGCTCGAGGGCTGACCAAGGCGGGGGCCA |
| | | AGAAGAAGGCGCGGAAGGAGGCCGCACTGGC |
| | | GGCAGAGGCGGAGGTGGAGGTGGAAGAGGTCC |
| | | CCAACACCATCCTCATCTGCATGGTGATCCTGC |
| | | TGAACATCGGCCTGGCCATCCTCTTTGTTCACC |
| | | TCCTGACCTGA (SEQ ID NO: 201) |
| Human | MSAATHSPMMQVASGNGDRDPL | ATGAGCGCCGCCACCCACTCGCCCATGATGCA |
| BAG3 | PPGWEIKIDPQTGWPFFVDHNSRT | GGTGGCGTCCGGCAACGGTGACCGCGACCCTTT |
| | TTWNDPRVPSEGPKETPSSANGPS | GCCCCCCGGATGGGAGATCAAGATCGACCCGC |
| | REGSRLPPAREGHPVYPQLRPGYI | AGACCGGCTGGCCCTTCTTCGTGGACCACAACA |
| | PIPVLHEGAENRQVHPFHVYPQPG | GCCGCACCACTACGTGGAACGACCCGCGCGTG |
| | MQRFRTEAAAAAPQRSQSPLRGM | CCCTCTGAGGGCCCCAAGGAGACTCCATCCTCT |
| | PETTQPDKQCGQVAAAAAAQPPA | GCCAATGGCCCTTCCCGGGAGGGCTCTAGGCTG |
| | SHGPERSQSPAASDCSSSSSSASLP | CCGCCTGCTAGGGAAGGCCACCCTGTGTACCCC |
| | SSGRSSLGSHQLPRGYISIPVIHEQ | CAGCTCCGACCAGGCTACATTCCCATTCCTGTG |
| | NVTRPAAQPSFHQAQKTHYPAQQ | CTCCATGAAGGCGCTGAGAACCGGCAGGTGCA |
| | GEYQTHQPVYHKIQGDDWEPRPL | CCCTTTCCATGTCTATCCCCAGCCTGGGATGCA |
| | RAASPFRSSVQGASSREGSPARSS | GCGATTCCGAACTGAGGCGGCAGCAGCGGCTC |
| | TPLHSPSPIRVHTVVDRPQQPMTH | CTCAGAGGTCCCAGTCACCTCTGCGGGGCATGC |
| | RETAPVSQPENKPESKPGPVGPEL | CAGAAACCACTCAGCCAGATAAACAGTGTGGA |
| | PPGHIPIQVIRKEVDSKPVSQKPPP | CAGGTGGCAGCGGCGGCGGCAGCCCAGCCCCC |
| | PSEKVEVKVPPAPVPCPPPSPGPS | AGCCTCCCACGGACCTGAGCGGTCCCAGTCTCC |
| | AVPSSPKSVATEERAAPSTAPAEA | AGCTGCCTCTGACTGCTCATCCTCATCCTCCTC |
| | TPPKPGEAEAPPKHPGVLKVEAIL | GGCCAGCCTGCCTTCCTCCGGCAGGAGCAGCCT |
| | EKVQGLEQAVDNFEGKKTDKKY | GGGCAGTCACCAGCTCCCGCGGGGGTACATCT |
| | LMIEEYLTKELLALDSVDPEGRA | CCATTCCGGTGATACACGAGCAGAACGTTACCC |
| | DVRQARRDGVRKVQTILEKLEQK | GGCCAGCAGCCCAGCCCTCCTTCCACCAAGCCC |
| | AIDVPGQVQVYELQPSNLEADQP | AGAAGACGCACTACCCAGCGCAGCAGGGGGAG |
| | LQAIMEMGAVAADKGKKNAGN | TACCAGACCCACCAGCCTGTGTACCACAAGATC |
| | AEDPHTETQQPEATAAATSNPSS | CAGGGGGATGACTGGGAGCCCCGGCCCCTGCG |
| | MTDTPGNPAAP (SEQ ID NO: 204) | GGCGGCATCCCCGTTCAGGTCATCTGTCCAGGG |
| | | TGCATCGAGCCGGGAGGGCTCACCAGCCAGGA |
| | | GCAGCACGCCACTCCACTCCCCCTCGCCCATCC |
| | | GTGTGCACACCGTGGTCGACAGGCCTCAGCAG |
| | | CCCATGACCCATCGAGAAACTGCACCTGTTTCC |
| | | CAGCCTGAAAACAAACCAGAAAGTAAGCCAGG |
| | | CCCAGTTGGACCAGAACTCCCTC |
| | | CTGGACACATCCCAATTCAAGTGATCCGCAAA |
| | | GAGGTGGATTCTAAACCTGTTTCCCAGAAGCCC |
| | | CCACCTCCCTCTGAGAAGGTAGAGGTGAAAGT |
| | | TCCCCCTGCTCCAGTTCCTTGTCCTCCTCCCAGC |
| | | CCTGGCCCTTCTGCTGTCCCCTCTTCCCCCAAG |
| | | AGTGTGGCTACAGAAGAGAGGGCAGCCCCCAG |
| | | CACTGCCCCTGCAGAAGCTACACCTCCAAAACC |
| | | AGGAGAAGCCGAGGCTCCCCCAAAACATCCAG |
| | | GAGTGCTGAAAGTGGAAGCCATCCTGGAGAAG |
| | | GTACAGGGGCTGGAGCAGGCTGTAGACAACTT |
| | | TGAAGGCAAGAAGACTGACAAAAAG |
| | | TACCTGATGATCGAAGAGTATTTGACCAAAGA |
| | | GCTGCTGGCCCTGGATTCAGTGGACCCCGAGG |
| | | GACGAGCCGATGTGCGTCAGGCCAGGAGAGAC |
| | | GGTGTCAGGAAGGTTCAGACCATCTTGGAAAA |
| | | ACTTGAACAGAAAGCCATTGATGTCCCAGGTC |
| | | AAGTCCAGGTCTATGAACTCCAGCCCAGCAAC |
| | | CTTGAAGCAGATCAGCCACTGCAGGCAATCAT |
| | | GGAGATGGGTGCCGTGGCAGCAGACAAGGGCA |
| | | AGAAAAATGCTGGAAATGCAGAAGATCCCCAC |
| | | ACAGAAACCCAGCAGCCAGAAGCCACAGCAGC |
| | | AGCGACTTCAAACCCCAGCAGCATGACAGACA |
| | | CCCCTGGTAACCCAGCAGCACCGTAG (SEQ ID |
| | | NO: 203) |
| Human | MDIAIHHPWIRRPFFPFHSPSRLFD | ATGGACATCGCCATCCACCACCCCTGGATCCGC |
| CRYAB | QFFGEHLLESDLFPTSTSLSPFYLR | CGCCCCTTCTTTCCTTTCCACTCCCCCAGCCGCC |
| | PPSFLRAPSWFDTGLSEMRLEKDR | TCTTTGACCAGTTCTTCGGAGAGCACCTGTTGG |
| | FSVNLDVKHFSPEELKVKVLGDVI | AGTCTGATCTTTTCCCGACGTCTACTTCCCTGA |
| | EVHGKHEERQDEHGFISREFHRK | GTCCCTTCTACCTTCGGCCACCCTCCTTCCTGCG |
| | YRIPADVDPLTITSSLSSDGVLTVN | GGCACCCAGCTGGTTTGACACTGGACTCTCAGA |
| | GPRKQVSGPERTIPITREEKPAVT | GATGCGCCTGGAGAAGGACAGGTTCTCTGTCA |
| | AAPKK (SEQ ID NO: 206) | ACCTGGATGTGAAGCACTTCTCCCCAGAGGAA |
| | | CTCAAAGTTAAGGTGTTGGGAGATGTGATTGA |
| | | GGTGCATGGAAAACATGAAGAGCGCCAGGATG |
| | | AACATGGTTTCATCTCCAGGGAGTTCCACAGGA |
| | | AATACCGGATCCCAGCTGATGTAGACCCTCTCA |
| | | CCATTACTTCATCCCTGTCATCTGATGGGGTCC |
| | | TCACTGTGAATGGACCAAGGAAACAGGTCTCT |
| | | GGCCCTGAGCGCACCATTCCCATCACCCGTGAA |
| | | GAGAAGCCTGCTGTCACCGCAGCCCCCAAGAA |
| | | ATAG (SEQ ID NO: 205) |
| Human | METPSQRRATRSGAQASSTPLSPT | ATGGAGACCCCGTCCCAGCGGCGCGCCACCCG |
| LMNA | RITRLQEKEDLQELNDRLAVYIDR | CAGCGGGGCGCAGGCCAGCTCCACTCCGCTGT |
| LaminA | VRSLETENAGLRLRITESEEVVSR | CGCCCACCCGCATCACCCGGCTGCAGGAGAAG |
| | EVSGIKAAYEAELGDARKTLDSV | GAGGACCTGCAGGAGCTCAATGATCGCTTGGC |
| | AKERARLQLELSKVREEFKELKA | GGTCTACATCGACCGTGTGCGCTCGCTGGAAAC |
| | RNTKKEGDLIAAQARLKDLEALL | GGAGAACGCAGGGCTGCGCCTTCGCATCACCG |
| | NSKEAALSTALSEKRTLEGELHDL | AGTCTGAAGAGGTGGTCAGCCGCGAGGTGTCC |
| | RGQVAKLEAALGEAKKQLQDEM | GGCATCAAGGCCGCCTACGAGGCCGAGCTCGG |
| | LRRVDAENRLQTMKEELDFQKNI | GGATGCCCGCAAGACCCTTGACTCAGTAGCCA |
| | YSEELRETKRRHETRLVEIDNGKQ | AGGAGCGCGCCCGCCTGCAGCTGGAGCTGAGC |
| | REFESRLADALQELRAQHEDQVE | AAAGTGCGTGAGGAGTTTAAGGAGCTGAA |
| | QYKKELEKTYSAKLDNARQSAER | AGCGCGCAATACCAAGAAGGAGGGTGACCTGA |
| | NSNLVGAAHEELQQSRIRIDSLSA | TAGCTGCTCAGGCTCGGCTGAAGGACCTGGAG |
| | QLSQLQKQLAAKEAKLRDLEDSL | GCTCTGCTGAACTCCAAGGAGGCCGCACTGAG |
| | ARERDTSRRLLAEKEREMAEMRA | CACTGCTCTCAGTGAGAAGCGCACGCTGGAGG |
| | RMQQQLDEYQELLDIKLALDMEI | GCGAGCTGCATGATCTGCGGGGCCAGGTGGCC |
| | HAYRKLLEGEEERLRLSPSPTSQR | AAGCTTGAGGCAGCCCTAGGTGAGGCCAAGAA |
| | SRGRASSHSSQTQGGGSVTKKRK | GCAACTTCAGGATGAGATGCTGCGGCGGGTGG |
| | LESTESRSSFSQHARTSGRVAVEE | ATGCTGAGAACAGGCTGCAGACCATGAAGGAG |
| | VDEEGKFVRLRNKSNEDQSMGN | GAACTGGACTTCCAGAAGAACATCTACAGTGA |
| | WQIKRQNGDDPLLTYRFPPKFTL | GGAGCTGCGTGAGACCAAGCGCCGTCATGAGA |
| | KAGQVVTIWAAGAGATHSPPTDL | CCCGACTGGTGGAGATTGACAATGGGAAGCAG |
| | VWKAQNTWGCGNSLRTALINST | CGTGAGTTTGAGAGCCGGCTGGCGGATGCGCT |
| | GEEVAMRKLVRSVTVVEDDEDE | GCAGGAACTGCGGGCCCAGCATGAGGACCAGG |
| | DGDDLLHHHHGSHCSSSGDPAEY | TGGAGCAGTATAAGAAGGAGCTGGAGAAGACT |
| | NLRSRTVLCGTCGQPADKASASG | TATTCTGCCAAGCTGGACAATGCCAGGCAGTCT |
| | SGAQVGGPISSGSSASSVTVTRSY | GCTGAGAGGAACAGCAACCTGGTGGGGGCTGC |
| | RSVGGSGGGSFGDNLVTRSYLLG | CCACGAGGAGCTGCAGCAGTCGCGCATCCGCA |
| | NSSPRTQSPQNCSIM (SEQ ID NO: | TCGACAGCCTCTCTGCCCAGCTCAGCCAGCTCC |
| | 208) | AGAAGCAGCTGGCAGCCAAGGAGGCGAAGCTT |
| | | CGAGACCTGGAGGACTCACTGGCCCGTGAGCG |
| | | GGACACCAGCCGGCGGCTGCTGGCGGAAAAGG |
| | | AGCGGGAGATGGCCGAGATGCGGGCAAGGATG |
| | | CAGCAGCAGCTGGACGAGTACCAGGAGCTTCT |
| | | GGACATCAAGCT |
| | | GGCCCTGGACATGGAGATCCACGCCTACCGCA |
| | | AGCTCTTGGAGGGCGAGGAGGAGAGGCTACGC |
| | | CTGTCCCCCAGCCCTACCTCGCAGCGCAGCCGT |
| | | GGCCGTGCTTCCTCTCACTCATCCCAGACACAG |
| | | GGTGGGGGCAGCGTCACCAAAAAGCGCAAACT |
| | | GGAGTCCACTGAGAGCCGCAGCAGCTTCTCAC |
| | | AGCACGCACGCACTAGCGGGCGCGTGGCCGTG |
| | | GAGGAGGTGGATGAGGAGGGCAAGTTTGTCCG |
| | | GCTGCGCAACAAGTCCAATGAGGACCAGTCCA |
| | | TGGGCAATTGGCAGATCAAGCGCCAGAATGGA |
| | | GATGATCCCTTGCTGACTTACCGGTTCC |
| | | CACCAAAGTTCACCCTGAAGGCTGGGCAGGTG |
| | | GTGACGATCTGGGCTGCAGGAGCTGGGGCCAC |
| | | CCACAGCCCCCCTACCGACCTGGTGTGGAAGG |
| | | CACAGAACACCTGGGGCTGCGGGAACAGCCTG |
| | | CGTACGGCTCTCATCAACTCCACTGGGGAAGA |
| | | AGTGGCCATGCGCAAGCTGGTGCGCTCAGTGA |
| | | CTGTGGTTGAGGACGACGAGGATGAGGATGGA |
| | | GATGACCTGCTCCATCACCACCACGGCTCCCAC |
| | | TGCAGCAGCTCGGGGGACCCCGCTGAGTACAA |
| | | CCTGCGCTCGCGCACCGTGCTGTGCGGGACCTG |
| | | CGGGCAGCCTGCCGACAAGGCATCTGCCAGCG |
| | | GCTCAGGAGCCCAGGTGGGCGGACCCATCTCC |
| | | TCTGGCTCTTCTGCCTCCAGTGTCACGGTCACT |
| | | CGCAGCTACCGCAGTGTGGGGGGCAGTGGGGG |
| | | TGGCAGCTTCGGGGACAATCTGGTCACCCGCTC |
| | | CTACCTCCTGGGCAACTCCAGCCCCCGAACCCA |
| | | GAGCCCCCAGAACTGCAGCATCATGTAA (SEQ |
| | | ID NO: 207) |
| Human | METPSQRRATRSGAQASSTPLSPT | ATGGAGACCCCGTCCCAGCGGCGCGCCACCCG |
| LMNA | RITRLQEKEDLQELNDRLAVYIDR | CAGCGGGGCGCAGGCCAGCTCCACTCCGCTGT |
| LaminC | VRSLETENAGLRLRITESEEVVSR | CGCCCACCCGCATCACCCGGCTGCAGGAGAAG |
| | EVSGIKAAYEAELGDARKTLDSV | GAGGACCTGCAGGAGCTCAATGATCGCTTGGC |
| | AKERARLQLELSKVREEFKELKA | GGTCTACATCGACCGTGTGCGCTCGCTGGAAAC |
| | RNTKKEGDLIAAQARLKDLEALL | GGAGAACGCAGGGCTGCGCCTTCGCATCACCG |
| | NSKEAALSTALSEKRTLEGELHDL | AGTCTGAAGAGGTGGTCAGCCGCGAGGTGTCC |
| | RGQVAKLEAALGEAKKQLQDEM | GGCATCAAGGCCGCCTACGAGGCCGAGCTCGG |
| | LRRVDAENRLQTMKEELDFQKNI | GGATGCCCGCAAGACCCTTGACTCAGTAGCCA |
| | YSEELRETKRRHETRLVEIDNGKQ | AGGAGCGCGCCCGCCTGCAGCTGGAGCTGAGC |
| | REFESRLADALQELRAQHEDQVE | AAAGTGCGTGAGGAGTTTAAGGAGCTGAA |
| | QYKKELEKTYSAKLDNARQSAER | AGCGCGCAATACCAAGAAGGAGGGTGACCTGA |
| | NSNLVGAAHEELQQSRIRIDSLSA | TAGCTGCTCAGGCTCGGCTGAAGGACCTGGAG |
| | QLSQLQKQLAAKEAKLRDLEDSL | GCTCTGCTGAACTCCAAGGAGGCCGCACTGAG |
| | ARERDTSRRLLAEKEREMAEMRA | CACTGCTCTCAGTGAGAAGCGCACGCTGGAGG |
| | RMQQQLDEYQELLDIKLALDMEI | GCGAGCTGCATGATCTGCGGGGCCAGGTGGCC |
| | HAYRKLLEGEEERLRLSPSPTSQR | AAGCTTGAGGCAGCCCTAGGTGAGGCCAAGAA |
| | SRGRASSHSSQTQGGGSVTKKRK | GCAACTTCAGGATGAGATGCTGCGGCGGGTGG |
| | LESTESRSSFSQHARTSGRVAVEE | ATGCTGAGAACAGGCTGCAGACCATGAAGGAG |
| | VDEEGKFVRLRNKSNEDQSMGN | GAACTGGACTTCCAGAAGAACATCTACAGTGA |
| | WQIKRQNGDDPLLTYRFPPKFTL | GGAGCTGCGTGAGACCAAGCGCCGTCATGAGA |
| | KAGQVVTIWAAGAGATHSPPTDL | CCCGACTGGTGGAGATTGACAATGGGAAGCAG |
| | VWKAQNTWGCGNSLRTALINST | CGTGAGTTTGAGAGCCGGCTGGCGGATGCGCT |
| | GEEVAMRKLVRSVTVVEDDEDE | GCAGGAACTGCGGGCCCAGCATGAGGACCAGG |
| | DGDDLLHHHHVSGSRR(SEQ ID | TGGAGCAGTATAAGAAGGAGCTGGAGAAGACT |
| | NO: 210) | TATTCTGCCAAGCTGGACAATGCCAGGCAGTCT |
| | | GCTGAGAGGAACAGCAACCTGGTGGGGGCTGC |
| | | CCACGAGGAGCTGCAGCAGTCGCGCATCCGCA |
| | | TCGACAGCCTCTCTGCCCAGCTCAGCCAGCTCC |
| | | AGAAGCAGCTGGCAGCCAAGGAGGCGAAGCTT |
| | | CGAGACCTGGAGGACTCACTGGCCCGTGAGCG |
| | | GGACACCAGCCGGCGGCTGCTGGCGGAAAAGG |
| | | AGCGGGAGATGGCCGAGATGCGGGCAAGGATG |
| | | CAGCAGCAGCTGGACGAGTACCAGGAGCTTCT |
| | | GGACATCAAGCTGGCCCTGGACATGGAGATCC |
| | | ACGCCTACCGCAAGCTCTTGGAGGGCGAGGAG |
| | | GAGAGGCTACGCCTGTCCCCCAGCCCTACCTCG |
| | | CAGCGCAGCCGTGGCCGTGCTTCCTCTCACTCA |
| | | TCCCAGACACAGGGTGGGGGCAGCGTCACCAA |
| | | AAAGCGCAAACTGGAGTCCACTGAGAGCCGCA |
| | | GCAGCTTCTCACAGCACGCACGCACTAGCGGG |
| | | CGCGTGGCCGTGGAGGAGGTGGATGAGGAGGG |
| | | CAAGTTTGTCCGGCTGCGCAACAAGTCCAATGA |
| | | GGACCAGTCCATGGGCAATTGGCAGATCAAGC |
| | | GCCAGAATGGAGATGATCCCTTGCTGACTTACC |
| | | GGTTCC |
| | | CACCAAAGTTCACCCTGAAGGCTGGGCAGGTG |
| | | GTGACGATCTGGGCTGCAGGAGCTGGGGCCAC |
| | | CCACAGCCCCCCTACCGACCTGGTGTGGAAGG |
| | | CACAGAACACCTGGGGCTGCGGGAACAGCCTG |
| | | CGTACGGCTCTCATCAACTCCACTGGGGAAGA |
| | | AGTGGCCATGCGCAAGCTGGTGCGCTCAGTGA |
| | | CTGTGGTTGAGGACGACGAGGATGAGGATGGA |
| | | GATGACCTGCTCCATCACCACCACGTGAGTGGT |
| | | AGCCGCCGCTGA (SEQ ID NO: 209) |
| Human | MADGSSDAAREPRPAPAPIRRRSS | ATGGCGGATGGGAGCAGCGATGCGGCTAGGGA |
| TNNI3 | NYRAYATEPHAKKKSKISASRKL | ACCTCGCCCTGCACCAGCCCCAATCAGACGCCG |
| | QLKTLLLQIAKQELEREAEERRGE | CTCCTCCAACTACCGCGCTTATGCCACGGAGCC |
| | KGRALSTRCQPLELAGLGFAELQ | GCACGCCAAGAAAAAATCTAAGATCTCCGCCT |
| | DLCRQLHARVDKVDEERYDIEAK | CGAGAAAATTGCAGCTGAAGACTCTGCTGCTG |
| | VTKNITEIADLTQKIFDLRGKFKR | CAGATTGCAAAGCAAGAGCTGGAGCGAGAGGC |
| | PTLRRVRISADAMMQALLGARAK | GGAGGAGCGGCGCGGAGAGAAGGGGCGCGCT |
| | ESLDLRAHLKQVKKEDTEKENRE | CTGAGCACCCGCTGCCAGCCGCTGGAGTTGGCC |
| | VGDWRKNIDALSGMEGRKKKFE | GGGCTGGGCTTCGCGGAGCTGCAGGACTTGTG |
| | S (SEQ ID NO: 212) | CCGACAGCTCCACGCCCGTGTGGACAAGGTGG |
| | | ATGAAGAGAGATACGACATAGAGGCAAAAGTC |
| | | ACCAAGAACATCACGGAGATTGCAGATCTGAC |
| | | TCAGAAGATCTTTGACCTTCGAGGCAAGTTTAA |
| | | GCGGCCCACCCTGCGGAGAGTGAGGATCTCTG |
| | | CAGATGCCATGATGCAGGCGCTGCTGGGGGCC |
| | | CGGGCTAAGGAGTCCCTGGACCTGCGGGCCCA |
| | | CCTCAAGCAGGTGAAGAAGGAGGACACCGAGA |
| | | AGGAAAACCGGGAGGTGGGAGACTGGCGCAA |
| | | GAACATCGATGCACTGAGTGGAATGGAGGGCC |
| | | GCAAGAAAAAGTTTGAGAGCTGA |
| | | (SEQ ID NO: 211) |
| Human | MVCFRLFPVPGSGLVLVCLVLGA | ATGGTGTGCTTCCGCCTCTTCCCGGTTCCGGGC |
| LAMP2a | VRSYALELNLTDSENATCLYAKW | TCAGGGCTCGTTCTGGTCTGCCTAGTCCTGGGA |
| | QMNFTVRYETTNKTYKTVTISDH | GCTGTGCGGTCTTATGCATTGGAACTTAATTTG |
| | GTVTYNGSICGDDQNGPKIAVQF | ACAGATTCAGAAAATGCCACTTGCCTTTATGCA |
| | GPGFSWIANFTKAASTYSIDSVSF | AAATGGCAGATGAATTTCACAGTACGCTATGA |
| | SYNTGDNTTFPDAEDKGILTVDEL | AACTACAAATAAAACTTATAAAACTGTAACCA |
| | LAIRIPLNDLFRCNSLSTLEKNDV | TTTCAGACCATGGCACTGTGACATATAATGGAA |
| | VQHYWDVLVQAFVQNGTVSTNE | GCATTTGTGGGGATGATCAGAATGGTCCCAAA |
| | FLCDKDKTSTVAPTIHTTVPSPTT | ATAGCAGTGCAGTTCGGACCTGGCTTTTCCTGG |
| | TPTPKEKPEAGTYSVNNGNDTCL | ATTGCGAATTTTACCAAGGCAGCATCTACTTAT |
| | LATMGLQLNITQDKVASVININPN | TCAATTGACAGCGTCTCATTTTCCTACAACACT |
| | TTHSTGSCRSHTALLRLNSSTIKY | GGTGATAACACAACATTTCCTGATGCTGAAGAT |
| | LDFVFAVKNENRFYLKEVNISMY | AAAGGAATTCTTACTGTTGATGAACTTTTGGCC |
| | LVNGSVFSIANNNLSYWDAPLGS | ATCAGAATTCCATTGAATGACCTTTTTAGATGC |
| | SYMCNKEQTVSVSGAFQINTFDL | AATAGTTTATCAACTTTGGAAAAGAATGATGTT |
| | RVQPFNVTQGKYSTAQDCSADD | GTCCAACACTACTGGGATGTTCTTGTACAAGCT |
| | DNFLVPIAVGAALAGVLILVLLAY | TTTGTCCAAAATGGCACAGTGAGCACAAATGA |
| | FIGLKHHHAGYEQF | GTTCCTGTGTGATAAAGACAAAACTTCAACAGT |
| | (SEQ ID NO: 214) | GGCACCCACCATACACACCACTGTGCCATCTCC |
| | | TACTACAACACCTACTCCAAAGGAAAAACCAG |
| | | AAGCTGGAACCTATTCAGTTAATAATGGCAATG |
| | | ATACTTGTCTGCTGGCTACCATGGGGCTGCAGC |
| | | TGAACATCACTCAGGATAAGGTTGCTTCAGTTA |
| | | TTAACATCAACCCCAATACAACTCACTCCACAG |
| | | GCAGCTGCCGTTCTCACACTGCTCTACTTAGAC |
| | | TCAATAGCAGCACCATTAAGTATCTAGACTTTG |
| | | TCTTTGCTGTGAAAAATGAAAACCGATTTTATC |
| | | TGAAGGAAGTGAACATCAGCATGTATTTGGTTA |
| | | ATGGCTCCGTTTTCAGCATTGCAAATAACAATC |
| | | TCAGCTACTGGGATGCCCCCCTGGGAAGTTCTT |
| | | ATATGTGCAACAAAGAGCAGACTGTTTCAGTGT |
| | | CTGGAGCATTTCAGATAAATACCTTTGATCTAA |
| | | GGGTTCAGCCTTTCAATGTGACACAAGGAAAG |
| | | TATTCTACAGCTCAAGACTGCAGTGCAGATGAC |
| | | GACAACTTCCTTGTGCCCATAGCGGTGGGAGCT |
| | | GCCTTGGCAGGAGTACTTATTCTAGTGTTGCTG |
| | | GCTTATTTTATTGGTCTCAAGCACCATCATGCT |
| | | GGATATGAGCAATTTTAG (SEQ ID NO: 213) |
| Human | MVCFRLFPVPGSGLVLVCLVLGA | ATGGTGTGCTTCCGCCTCTTCCCGGTTCCGGGC |
| LAMP2b | VRSYALELNLTDSENATCLYAKW | TCAGGGCTCGTTCTGGTCTGCCTAGTCCTGGGA |
| | QMNFTVRYETTNKTYKTVTISDH | GCTGTGCGGTCTTATGCATTGGAACTTAATTTG |
| | GTVTYNGSICGDDQNGPKIAVQF | ACAGATTCAGAAAATGCCACTTGCCTTTATGCA |
| | GPGFSWIANFTKAASTYSIDSVSF | AAATGGCAGATGAATTTCACAGTACGCTATGA |
| | SYNTGDNTTFPDAEDKGILTVDEL | AACTACAAATAAAACTTATAAAACTGTAACCA |
| | LAIRIPLNDLFRCNSLSTLEKNDV | TTTCAGACCATGGCACTGTGACATATAATGGAA |
| | VQHYWDVLVQAFVQNGTVSTNE | GCATTTGTGGGGATGATCAGAATGGTCCCAAA |
| | FLCDKDKTSTVAPTIHTTVPSPTT | ATAGCAGTGCAGTTCGGACCTGGCTTTTCCTGG |
| | TPTPKEKPEAGTYSVNNGNDTCL | ATTGCGAATTTTACCAAGGCAGCATCTACTTAT |
| | LATMGLQLNITQDKVASVININPN | TCAATTGACAGCGTCTCATTTTCCTACAACACT |
| | TTHSTGSCRSHTALLRLNSSTIKY | GGTGATAACACAACATTTCCTGATGCTGAAGAT |
| | LDFVFAVKNENRFYLKEVNISMY | AAAGGAATTCTTACTGTTGATGAACTTTTGGCC |
| | LVNGSVFSIANNNLSYWDAPLGS | ATCAGAATTCCATTGAATGACCTTTTTAGATGC |
| | SYMCNKEQTVSVSGAFQINTFDL | AATAGTTTATCAACTTTGGAAAAGAATGATGTT |
| | RVQPFNVTQGKYSTAQECSLDDD | GTCCAACACTACTGGGATGTTCTTGTACAAGCT |
| | TILIPIIVGAGLSGLIIVIVIAYVIGR | TTTGTCCAAAATGGCACAGTGAGCACAAATGA |
| | RKSYAGYQTL (SEQ ID NO: 216) | GTTCCTGTGTGATAAAGACAAAACTTCAACAGT |
| | | GGCACCCACCATACACACCACTGTGCCATCTCC |
| | | TACTACAACACCTACTCCAAAGGAAAAACCAG |
| | | AAGCTGGAACCTATTCAGTTAATAATGGCAATG |
| | | ATACTTGTCTGCTGGCTACCATGGGGCTGCAGC |
| | | TGAACATCACTCAGGATAAGGTTGCTTCAGTTA |
| | | TTAACATCAACCCCAATACAACTCACTCCACAG |
| | | GCAGCTGCCGTTCTCACACTGCTCTACTTAGAC |
| | | TCAATAGCAGCACCATTAAGTATCTAGACTTTG |
| | | TCTTTGCTGTGAAAAATGAAAACCGATTTTATC |
| | | TGAAGGAAGTGAACATCAGCATGTATTTGGTTA |
| | | ATGGCTCCGTTTTCAGCATTGCAAATAACAATC |
| | | TCAGCTACTGGGATGCCCCCCTGGGAAGTTCTT |
| | | ATATGTGCAACAAAGAGCAGACTGTTTCAGTGT |
| | | CTGGAGCATTTCAGATAAATACCTTTGATCTAA |
| | | GGGTTCAGCCTTTCAATGTGACACAAGGAAAG |
| | | TATTCTACAGCCCAAGAGTGTTCGCTGGATGAT |
| | | GACACCATTCTAATCCCAATTATAGTTGGTGCT |
| | | GGTCTTTCAGGCTTGATTATCGTTATAGTGATT |
| | | GCTTACGTAATTGGCAGA |
| | | AGAAAAAGTTATGCTGGATATCAGACTCTGTA |
| | | A (SEQ ID NO: 215) |
| Human | MVCFRLFPVPGSGLVLVCLVLGA | ATGGTGTGCTTCCGCCTCTTCCCGGTTCCGGGC |
| LAMP2c | VRSYALELNLTDSENATCLYAKW | TCAGGGCTCGTTCTGGTCTGCCTAGTCCTGGGA |
| | QMNFTVRYETTNKTYKTVTISDH | GCTGTGCGGTCTTATGCATTGGAACTTAATTTG |
| | GTVTYNGSICGDDQNGPKIAVQF | ACAGATTCAGAAAATGCCACTTGCCTTTATGCA |
| | GPGFSWIANFTKAASTYSIDSVSF | AAATGGCAGATGAATTTCACAGTACGCTATGA |
| | SYNTGDNTTFPDAEDKGILTVDEL | AACTACAAATAAAACTTATAAAACTGTAACCA |
| | LAIRIPLNDLFRCNSLSTLEKNDV | TTTCAGACCATGGCACTGTGACATATAATGGAA |
| | VQHYWDVLVQAFVQNGTVSTNE | GCATTTGTGGGGATGATCAGAATGGTCCCAAA |
| | FLCDKDKTSTVAPTIHTTVPSPTT | ATAGCAGTGCAGTTCGGACCTGGCTTTTCCTGG |
| | TPTPKEKPEAGTYSVNNGNDTCL | ATTGCGAATTTTACCAAGGCAGCATCTACTTAT |
| | LATMGLQLNITQDKVASVININPN | TCAATTGACAGCGTCTCATTTTCCTACAACACT |
| | TTHSTGSCRSHTALLRLNSSTIKY | GGTGATAACACAACATTTCCTGATGCTGAAGAT |
| | LDFVFAVKNENRFYLKEVNISMY | AAAGGAATTCTTACTGTTGATGAACTTTTGGCC |
| | LVNGSVFSIANNNLSYWDAPLGS | ATCAGAATTCCATTGAATGACCTTTTTAGATGC |
| | SYMCNKEQTVSVSGAFQINTFDL | AATAGTTTATCAACTTTGGAAAAGAATGATGTT |
| | RVQPFNVTQGKYSTAEECSADSD | GTCCAACACTACTGGGATGTTCTTGTACAAGCT |
| | LNFLIPVAVGVALGFLIIVVFISYM | TTTGTCCAAAATGGCACAGTGAGCACAAATGA |
| | IGRRKSRTGYQSV | GTTCCTGTGTGATAAAGACAAAACTTCAACAGT |
| | (SEQ ID NO: 218) | GGCACCCACCATACACACCACTGTGCCATCTCC |
| | | TACTACAACACCTACTCCAAAGGAAAAACCAG |
| | | AAGCTGGAACCTATTCAGTTAATAATGGCAATG |
| | | ATACTTGTCTGCTGGCTACCATGGGGCTGCAGC |
| | | TGAACATCACTCAGGATAAGGTTGCTTCAGTTA |
| | | TTAACATCAACCCCAATACAACTCACTCCACAG |
| | | GCAGCTGCCGTTCTCACACTGCTCTACTTAGAC |
| | | TCAATAGCAGCACCATTAAGTATCTAGACTTTG |
| | | TCTTTGCTGTGAAAAATGAAAACCGATTTTATC |
| | | TGAAGGAAGTGAACATCAGCATGTATTTGGTTA |
| | | ATGGCTCCGTTTTCAGCATTGCAAATAACAATC |
| | | TCAGCTACTGGGATGCCCCCCTGGGAAGTTCTT |
| | | ATATGTGCAACAAAGAGCAGACTGTTTCAGTGT |
| | | CTGGAGCATTTCAGATAAATACCTTTGATCTAA |
| | | GGGTTCAGCCTTTCAATGTGACACAAGGAAAG |
| | | TATTCTACAGCTGAAGAATGTTCTGCTGACTCT |
| | | GACCTCAACTTTCTTATTCCTGTTGCAGTGGGT |
| | | GTGGCCTTGGGCTTCCTTATAATTGTTGTCTTTA |
| | | TCTCTTATATGATTGGAAGAAGGAAAAGTCGTA |
| | | CTGGTTATCAGTCTGTGTAA (SEQ ID NO: 217) |
| Human | MSCNGGSHPRINTLGRMIRAESGP | ATGAGCTGCAACGGAGGCTCCCACCCGCGGAT |
| DSP_DPI | DLRYEVTSGGGGTSRMYYSRRG | CAACACTCTGGGCCGCATGATCCGCGCCGAGTC |
| | VITDQNSDGYCQTGTMSRHQNQ | TGGCCCGGACCTGCGCTACGAGGTGACCAGCG |
| | NTIQELLQNCSDCLMRAELIVQPE | GCGGCGGGGGCACCAGCAGGATGTACTATTCT |
| | LKYGDGIQLTRSRELDECFAQAN | CGGCGCGGCGTGATCACCGACCAGAACTCGGA |
| | DQMEILDSLIREMRQMGQPCDAY | CGGCTACTGTCAAACCGGCACGATGTCCAGGC |
| | QKRLLQLQEQMRALYKAISVPRV | ACCAGAACCAGAACACCATCCAGGAGCTGCTG |
| | RRASSKGGGGYTCQSGSGWDEFT | CAGAACTGCTCCGACTGCTTGATGCGAGCAGA |
| | KHVTSECLGWMRQQRAEMDMV | GCTCATCGTGCAGCCTGAATTGAAGTATGGAG |
| | AWGVDLASVEQHINSHRGIHNSI | ATGGAATACAACTGACTCGGAGTCGAGAATTG |
| | GDYRWQLDKIKADLREKSAIYQL | GATGAGTGTTTTGCCCAGGCCAATGACCA |
| | EEEYENLLKASFERMDHLRQLQN | AATGGAAATCCTCGACAGCTTGATCAGAGAGA |
| | IIQATSREIMWINDCEEEELLYDW | TGCGGCAGATGGGCCAGCCCTGTGATGCTTACC |
| | SDKNTNIAQKQEAFSIRMSQLEVK | AGAAAAGGCTTCTTCAGCTCCAAGAGCAAATG |
| | EKELNKLKQESDQLVLNQHPASD | CGAGCCCTTTATAAAGCCATCAGTGTCCCTCGA |
| | KIEAYMDTLQTQWSW | GTCCGCAGGGCCAGCTCCAAGGGTGGTGGAGG |
| | ILQITKCIDVHLKENAAYFQFFEE | CTACACTTGTCAGAGTGGCTCTGGCTGGGATGA |
| | AQSTEAYLKGLQDSIRKKYPCDK | GTTCACCAAACATGTCACCAGTGAATGTTTGGG |
| | NMPLQHLLEQIKELEKEREKILEY | GTGGATGAGGCAGCAAAGGGCGGAGATGGACA |
| | KRQVQNLVNKSKKIVQLKPRNPD | TGGTGGCCTGGGGTGTGGACCTGGCCTCAGTGG |
| | YRSNKPIILRALCDYKQDQKIVHK | AGCAGCACATTAACAGCCACCGGGGCATCCAC |
| | GDECILKDNNERSKWYVTGPGGV | AACTCCATCGGCGACTATCGCTGGC |
| | DMLVPSVGLIIPPPNPLAVDLSCKI | AGCTGGACAAAATCAAAGCCGACCTGCGCGAG |
| | EQYYEAILALWNQLYINMKSLVS | AAATCTGCGATCTACCAGTTGGAGGAGGAGTA |
| | WHYCMIDIEKIRAMTIAKLKTMR | TGAAAACCTGCTGAAAGCGTCCTTTGAGAGGA |
| | QEDYMKTIADLELHYQEFIRNSQ | TGGATCACCTGCGACAGCTGCAGAACATCATTC |
| | GSEMFGDDDKRKIQSQFTDAQKH | AGGCCACGTCCAGGGAGATCATGTGGATCAAT |
| | YQTLVIQLPGYPQHQTVTTTEITH | GACTGCGAGGAGGAGGAGCTGCTGTACGACTG |
| | HGTCQDVNHNKVIETNRENDKQE | GAGCGACAAGAACACCAACATCGCTCAGAAAC |
| | TWMLMELQKIRRQIEHCEGRMTL | AGGAGGCCTTCTCCATACGCATGAGTCAACTGG |
| | KNLPLADQGSSHHITVKINELKSV | AAGTTAAAGAAAAAGAGCTCAATAAGCTGAAA |
| | QNDSQAIAEVLNQLKDMLANFRG | CAAGAAAGTGACCAACTTGTCCTCAATCAGCAT |
| | SEKYCYLQNEVFGLFQKLENING | CCAGCTTCAGACAAAATTGAGGCCTATATGGA |
| | VTDGYLNSLCTVRALLQAILQTE | CACTCTGCAGACGCAGTGGAGTTGGATTCTTCA |
| | DMLKVYEARLTEEETVCLDLDKV | GATCACCAAGTGCATTGATGTTCATCTGAAAGA |
| | EAYRCGLKKIKNDLNLKKSLLAT | AAATGCTGCCTACTTTCAGTTTTTTGAAGAGGC |
| | MKTELQKAQQIHS | GCAGTCTACTGAAGCATACCTGAAGGGGCTCC |
| | QTSQQYPLYDLDLGKFGEKVTQL | AGGACTCCATCAGGAAGAAGTACCCCTGCGAC |
| | TDRWQRIDKQIDFRLWDLEKQIK | AAGAACATGCCCCTGCAGCACCTGCTGGAACA |
| | QLRNYRDNYQAFCKWLYDAKRR | GATCAAGGAGCTGGAGAAAGAACGAGAGAAA |
| | QDSLESMKFGDSNTVMRFLNEQK | ATCCTTGAATACAAGCGTCAGGTGCAGAACTTG |
| | NLHSEISGKRDKSEEVQKIAELCA | GTAAACAAGTCTAAGAAGATTGTACAGCTGAA |
| | NSIKDYELQLASYTSGLETLLNIPI | GCCTCGTAACCCAGACTACAGAAGCAATAAAC |
| | KRTMIQSPSGVILQEAADVHARYI | CCATTATTCTCAGAGCTCTCTG |
| | ELLTRSGDYYRFLSEMLKSLEDL | TGACTACAAACAAGATCAGAAAATCGTGCATA |
| | KLKNTKIEVLEEERLARDANSEN | AGGGGGATGAGTGTATCCTGAAGGACAACAAC |
| | CNKNKFLDQNLQKYQAECSQFK | GAGCGCAGCAAGTGGTACGTGACGGGCCCGGG |
| | AKLASLEELKRQAELDGKSAKQN | AGGCGTTGACATGCTTGTTCCCTCTGTGGGGCT |
| | LDKCYGQIKELNEKITRLTYEIED | GATCATCCCTCCTCCGAACCCACTGGCCGTGGA |
| | EKRRRKSVEDRFDQQKNDYDQL | CCTCTCTTGCAAGATTGAGCAGTACTACGAAGC |
| | QKARQCEKENLGWQKLESEKAIK | CATCTTGGCTCTGTGGAACCAGCTCTACATCAA |
| | EKEYEIERLRVLLQEEGTRKREYE | CATGAAGAGCCTGGTGTCCTGGCACTACTGCAT |
| | NELAKVRNHYNEEMSNLENKYE | GATTGACATAGAGAAGATCAGGGCCATGACAA |
| | TEINITKTTIKEISMQKEDDSKNLR | TCGCCAAGCTGAAAACAATGCGGCAGGAAGAT |
| | NQLDRLSRENRDLKDEIVRLNDSI | TACATGAAGACGATAGCCGACCTTGAGTTACAT |
| | LQATEQRRRAEENALQQKACGSE | TACCAAGAGTTCATCAGAAATAGCCAAGGCTC |
| | IMQKKQHLEIELKQVMQQRSEDN | AGAGATGTTTGGAGATGATGACAAGCGGAAAA |
| | ARHKQSLEEAAKTIQDKNKEIERL | TACAGTCTCAGTTCACCGATGCCCAGAAGCATT |
| | KAEFQEEAKRRWEYENELSKVRN | ACCAGACCCTGGTCATTCAGCTCCCTGGCTATC |
| | NYDEEIISLKNQFETEINITKTTIHQ | CCCAGCACCAGACAGTGACCACAACTGAAATCC |
| | LTMQKEEDTSGYRAQIDNLTREN | ACTCATCATGGAACCTGCCAAGATGTCAACCAT |
| | RSLEEIKRLKNTLTQTTENLRRV | AATAAAGTAATTGAAACCAACAGAGAAAATGA |
| | EEDIQQQKATGSEVSQRKQQLEV | CAAGCAAGAAACATGGATGCTGATGGAGCTGC |
| | ELRQVTQMRTEESVRYKQSLDDA | AGAAGATTCGCAGGCAGATAGAGCACTGCGAG |
| | AKTIQDKNKEIERLKQLIDKETND | GGCAGGATGACTCTCAAAAACCTCCCTCTAGCA |
| | RKCLEDENARLQRVQYDLQKAN | GACCAGGGATCTTCTCACCACATCACAGTGAA |
| | SSATETINKLKVQEQELTRLRIDY | AATTAACGAGCTTAAGAGTGTGCAGAATGATT |
| | ERVSQERTVKDQDITRFQNSLKEL | CACAAGCAATTGCTGAGGTTCTCAACCAGCTTA |
| | QLQKQKVEEELNRLKRTASEDSC | AAGATATGCTTGCCAACTTCAGAGGTTCTGAAA |
| | KRKKLEEELEGMRRSLKEQAIKIT | AGTACTGCTATTTACAGAATGAAGTATTTGGAC |
| | NLTQQLEQASIVKKRSEDDLRQQ | TATTTCAGAAACTGGAAAATATCAATGGTGTTA |
| | RDVLDGHLREKQRTQEELRRLSS | CAGATGGCTACTTAAATAGCTTATGCACAGTAA |
| | EVEALRRQLLQEQESVKQAHLRN | GGGCACTGCTCCAGGCTATTCTCCAAACAGAA |
| | EHFQKAIEDKSRSLNESKIEIERLQ | GACATGTTAAAGGTTTATGAAGCCAGGCTCACT |
| | SLTENLTKEHLMLEEELRNLRLEY | GAGGAGGAAACTGTCTGCCTGGACCTGGATAA |
| | DDLRRGRSEADSDKNATILELRSQ | AGTGGAAGCTTACCGCTGTGGACTGAAGAAAA |
| | LQISNNRTLELQGLINDLQREREN | TAAAAAATGA |
| | LRQEIEKFQKQALEASNRIQESKN | CTTGAACTTGAAGAAGTCGTTGTTGGCCACTAT |
| | QCTQVVQERESLLVKIKVLEQDK | GAAGACAGAACTACAGAAAGCCCAGCAGATCC |
| | ARLQRLEDELNRAKSTLEAETRV | ACTCTCAGACTTCACAGCAGTATCCACTTTATG |
| | KQRLECEKQQIQNDLNQWKTQY | ATCTGGACTTGGGCAAGTTCGGTGAAAAAGTC |
| | SRKEEAIRKIESEREKSEREKNSLR | ACACAGCTGACAGACCGCTGGCAAAGGATAGA |
| | SEIERLQAEIKRIEERCRRKLEDST | TAAACAGATCGACTTTAGGTTATGGGACCTGGA |
| | RETQSQLETERSRYQREIDKLRQR | GAAACAAATCAAGCAATTGAGGAATTATCGTG |
| | PYGSHRETQTECEWTVDTSKLVF | ATAACTATCAGGCTTTCTGCAAGTGGCTCTATG |
| | DGLRKKVTAMQLYECQLIDKTTL | ATGCTAAACGCCGCCAGGATTCCTTAGAATCCA |
| | DKLLKGKKSVEEVASEIQPFLRGA | TGAAATTTGGAGATTCCAACACAGTCATGCGGT |
| | GSIAGASASPKEKYSLVEAKRKK | TTTTGAATGAGCAGAAGAACTTGC |
| | LISPESTVMLLEAQAATGGIIDPHR | ACAGTGAAATATCTGGCAAACGAGACAAATCA |
| | NEKLTVDSAIARDLIDFDDRQQIY | GAGGAAGTACAAAAAATTGCTGAACTTTGCGC |
| | AAEKAITGFDDPFSGKTVSVSEAI | CAATTCAATTAAGGATTATGAGCTCCAGCTGGC |
| | KKNLIDRETGMRLLEAQIASGGV | CTCATACACCTCAGGACTGGAAACTCTGCTGAA |
| | VDPVNSVFLPKDVALARGLIDRD | CATACCTATCAAGAGGACCATGATTCAGTCCCC |
| | LYR | TTCTGGGGTGATTCTGCAAGAGGCTGCAGATGT |
| | SLNDPRDSQKNFVDPVTKKKVSY | TCATGCTCGGTACATTGAACTACTTACAAGATC |
| | VQLKERCRIEPHTGLLLLSVQKRS | TGGAGACTATTACAGGTTCTTAAGTGAGATGCT |
| | MSFQGIRQPVTVTELVDSGILRPS | GAAGAGTTTGGAAGATCTGAAGCTGAAAAATA |
| | TVNELESGQISYDEVGERIKDFLQ | CCAAGATCGAAGTTTTGGAAGAGGAGCTCAGA |
| | GSSCIAGIYNETTKQKLGIYEAMKI | CTGGCCCGAGATGCCAACTCGGAAAACTGTAA |
| | GLVRPGTALELLEAQAATGFIVDP | TAAGAACAAATTCCTGGATCAGAACCTGCAGA |
| | VSNLRLPVEEAYKRGLVGIEFKEK | AATACCAGGCAGAGTGTTCCCAGTTCAAAGCG |
| | LLSAERAVTGYNDPETGNIISLFQ | AAGCTTGCGAGCCTGGAGGAGCTGAAGAGACA |
| | AMNKELIEKGHGIRLLEAQIATGG | GGCTGAGCTGGATGGGAAGTCGGCTAAGCAAA |
| | IIDPKESHRLPVDIAYKRGYFNEEL | ATCTAGACAAGTGCTACGGCCAAATAAAAGAA |
| | SEILSDPSDDTKGFFDPNTEENLT | CTCAATGAGAAGATCACCCGACTGACTTATGA |
| | YLQLKERCIKDEETGLCLLPLKEK | GATTGAAGATGAAAAGAGAAGAAGAAAATCTG |
| | KKQVQTSQKNTLRKRRVVIVDPE | TGGAAGACAGATTTGACCAACAGAAGAATGAC |
| | TNKEMSVQEAYKKGLIDYETFKE | TATGACCAACTGCAGAAAGCAAGGCAATGTGA |
| | LCEQECEWEEITITGSDGSTRVVL | AAAGGAGAACCTTGGTTGGCAGAAATTAGAGT |
| | VDRKTGSQYDIQDAIDKGLVDRK | CTGAGAAAGCCATCAAGGAGAAGGAGTACGAG |
| | FFDQYRSGSLSLTQFADMISLKNG | ATTGAAAGGTTGAGGGTTCTACTGCAGGAAGA |
| | VGTSSSMGSGVSDDVFSSSRHESV | AGGCACCC |
| | SKISTISSVRNLTIRSSSFSDTLEES | GGAAGAGAGAATATGAAAATGAGCTGGCAAAG |
| | SPIAAIFDTENLEKISITEGIERGIV | GTAAGAAACCACTATAATGAGGAGATGAGTAA |
| | DSITGQRLLEAQACTGGIIHPTTG | TTTAAGGAACAAGTATGAAACAGAGATTAACA |
| | QKLSLQDAVSQGVIDQDMATRLK | TTACGAAGACCACCATCAAGGAGATATCCATG |
| | PAQKAFIGFEGVKGKKKMSAAEA | CAAAAAGAGGATGATTCCAAAAATCTTAGAAA |
| | VKEKWLPYEAGQRFLEFQYLTGG | CCAGCTTGATAGACTTTCAAGGGAAAATCGAG |
| | LVDPEVHGRISTEEAIRKGFIDGR | ATCTGAAGGATGAAATTGTCAGGCTCAATGAC |
| | AAQRLQDTSSYAKILTCPKTKLKI | AGCATCTTGCAGGCCACTGAGCAGCGAAGGCG |
| | SYKDAINRSMVEDITGLRLLEAAS | AGCTGAAGAAAACGCCCTTCAGCAAAAGGCCT |
| | VSSKGLPSPYNMSSAPGSRSGSRS | GTGGCTCTGAGATAATGCAGAAGAAGCAGCAT |
| | GSRSGSRSGSRSGSRRGSFDATGN | CTGGAGATAGAACTGAAGCAGGTCATGCAGCA |
| | SSYSYSYSFSSSSIGH (SEQ ID | GCGCTCTGAGGACAATGCCCGGCACAAGCAGT |
| | NO: 220) | CCCTGGAGGAGGCTGCCAAGACCATTCAGGAC |
| | | AAAAATAAGGAGATCGAGAGACTCAAAGCTGA |
| | | GTTTCAGGAGGAGGCCAAGCGCCGCTGGGAAT |
| | | ATGAAAATGAACTGAGTAAGGTAAGAAACAAT |
| | | TATGATGAGGAGATCATTAGCTTAAAAAATCA |
| | | GTTTGAGACCGAGATCAACATCACCAAGACCA |
| | | CCATCCACCAGCTCACCATGCAGAAGGAAGAG |
| | | GATACCAGTGGCTACCGGGCTCAGATAGACAA |
| | | TCTCACCCGAGAAAACAGGAGCTTATCTGAAG |
| | | AAATAAAGAGGCTGAAGAACACTCTAACCCAG |
| | | ACCACAGAGAATCTCAGGAGGGTGGAAGAAGA |
| | | CATCCAACAGCAAAAGGCCACTGGCTCTGAGG |
| | | TGTCTCAGAGGAAACAGCAGCTGGAGGTTGAG |
| | | CTGAGACAAGTCACTCAGATGCGAACAGAGGA |
| | | GAGCGTAAGATATAAGCAATCTCTTGATGATGC |
| | | TGCCAAAACCATCCAGGATAAAAACAAGGAGA |
| | | TAGAAAGGTTAAAACAACTGATCGACAAAGAA |
| | | ACAAATGACCGGAAATGCCTGGAAGATGAAAA |
| | | CGCGAGATTACAAAGGGTCCAGTATGACCTGC |
| | | AGAAAGCAAACAGTAGTGCGACGGAGACAATA |
| | | AACAAACTGAAGGTTCAGGAGCAAGAACTGAC |
| | | ACGCCTGAGGATCGACTATGAAAGGGTTTCCC |
| | | AGGAGAGGACTGTGAAGGACCAGGATATCACG |
| | | CGGTTCCAGAACTCTCTGAAAGAGCTGCAGCTG |
| | | CAGAAGCAGAAGGTGGAAGAGGAGCTGAATCG |
| | | GCTGAAGAGGACCGCGTCAGAAGACTCCTGCA |
| | | AGAGGAAGAAGCTGGAGGAAGAGCTGGAAGG |
| | | CATGAGGAGGTCGCTGAAGGAGCAAGCCATCA |
| | | AAATCACCAACCTGACCCAGCAGCTGGAGCAG |
| | | GCATCCATTGTTAAGAAGAGGAGTGAGGATGA |
| | | CCTCCGGCAGCAGAGGGACGTGCTGGATGGCC |
| | | ACCTGAGGGAAAAGCAGAGGACCCAGGAAGA |
| | | GCTGAGGAGGCTCTCTTCTGAGGTCGAGGCCCT |
| | | GAGGCGGCAGTTACTCCAGGAACAGGAAAGTG |
| | | TCAAACAAGCTCACTTGAGGAATGAGCATTTCC |
| | | AGAAGGCGATAGAAGATAAAAGCAGAAGCTTA |
| | | AATGAAAGCAAAATAGAAATTGAGAGGCTGCA |
| | | GTCTCTCACAGAGAACCTGACCAAGGAGCA |
| | | CTTGATGTTAGAAGAAGAACTGCGGAACCTGA |
| | | GGCTGGAGTACGATGACCTGAGGAGAGGACGA |
| | | AGCGAAGCGGACAGTGATAAAAATGCAACCAT |
| | | CTTGGAACTAAGGAGCCAGCTGCAGATCAGCA |
| | | ACAACCGGACCCTGGAACTGCAGGGGCTGATT |
| | | AATGATTTACAGAGAGAGAGGGAAAATTTGAG |
| | | ACAGGAAATTGAGAAATTCCAAAAGCAGGCTT |
| | | TAGAGGCATCTAATAGGATTCAGGAATCAAAG |
| | | AATCAGTGTACTCAGGTGGTACAGGAAAGAGA |
| | | GAGCCTTCTGGTGAAAATCAAAGTCCTGGAGC |
| | | AAGACAAGGCAAGGCTGCAGAGGCTGGAGGAT |
| | | GAGCTGAATCGTGCAAAATCAACTCTAGAGGC |
| | | AGAAACCAGGGTGAAACAGCGCCTGGAGTGTG |
| | | AGAAACAGCAAATTCAGAATGACCTGAATCAG |
| | | TGGAAGACTCAATATTCCCGCAAGGAGGAGGC |
| | | TATTAGGAAGATAGAATCGGAAAGAGAAAAGA |
| | | GTGAGAGAGAGAAGAACAGTCTTAGGAGTGAG |
| | | ATCGAAAGACTCCAAGCAGAGATCAAGAGAAT |
| | | TGAAGAGAGGTGCAGGCGTAAGCTGGAGGATT |
| | | CTACCAGGGAGACACAGTCACAGTTAGAAACA |
| | | GAACGCTCCCGATATCAGAGGGAGATTGATAA |
| | | ACTCAGACAGCGCCCATATGGGTCCCATCGAG |
| | | AGACCCAGACTGAGTGTGAGTGGACCGTTGAC |
| | | ACCTCCAAGCTGGTGTTTGATGGGCTGAGGAA |
| | | GAAGGTGACAGCAATGCAGCTCTATGAGTGTC |
| | | AGCTGATCGACAAAACAACCTTGGACAAACTA |
| | | TTGAAGGGGAAGAAGTCAGTGGAAGAAGTTGC |
| | | TTCTGAAATCCAGCCATTCCTTCGGGGTGCAGG |
| | | ATCTATCGCTGGAGCATCTGCTTCTCCTAAGGA |
| | | AAAATACTCTTTGGTAGAGGCCAAGAGAAAGA |
| | | AATTAATCAGCCCAGAATCCACAGTCATGCTTC |
| | | TGGAGGCCCAGGCAGCTACAGGTGGTATAATT |
| | | GATCCCCATCGGAATGAGAAGCTGACTGTCGA |
| | | CAGTGCCATAGCTCGGGACCTCATTGACTTCGA |
| | | TGACCGTCAGCAGATATATGCAGCAGAAAAAG |
| | | CTATCACTGGTTTTGATGATCCATTTTCAGGCA |
| | | AGACAGTATCTGTTTCAGAAGCCATCAAGAAA |
| | | AATTTGATTGATAGAGAAACCGGAATGCGCCT |
| | | GCTGGAAGCCCAGATTGCTTCAGGGGGTGTAG |
| | | TAGACCCTGTGAACAGTGTCTTTTTGCCAAAAG |
| | | ATGTCGCCTTGGCCCGGGGGCTGATTGATAGAG |
| | | ATTTGTATCGATCCCTGAATGATCCCCGAGATA |
| | | GTCAGAAAAACTTTGTGGATCCAGTCACCAAA |
| | | AAGAAGGTCAGTTACGTGCAGCTGAAGGAACG |
| | | GTGCAGAATCGAACCACATACTGGTCTGCTCTTG |
| | | CTTTCAGTACAGAAGAGAAGCATGTCCTTCCAA |
| | | GGAATCAGACAACCTGTGACCGTCACTGAGCT |
| | | AGTAGATTCTGGTATATTGAGACCGTCCACTGT |
| | | CAATGAACTGGAATCTGGTCAGATTTCTTATGA |
| | | CGAGGTTGGTGAGAGAATTAAGGACTTCCTCC |
| | | AGGGTTCAAGCTGCATAGCAGGCATATACAAT |
| | | GAGACCACAAAACAGAAGCTTGGCATTTATGA |
| | | GGCCATGAAAATTGGCTTAGTCCGACCTGGTAC |
| | | TGCTCTGGAGTTGCTGGAAGCCCAAGCAGCTAC |
| | | TGGCTTTATAGTGGATCCTGTTAGCAACTTGAG |
| | | GTTACCAGTGGAGGAAGCCTACAAGAGAGGTC |
| | | TGGTGGGCATTGAGTTCAAAGAGAAGCTCCTGT |
| | | CTGCAGAACGAGCTGTCACTGGGTATAATGATC |
| | | CTGAAACAGGAAACATCATCTCTTTGTTCCAAG |
| | | CCATGAATAAGGAACTCATCGAAAAGGGCCAC |
| | | GGTATTCGCTTATTAGAAGCACAGATCGCAACC |
| | | GGGGGGATCATTGACCCAAAGGAGAGCCATCG |
| | | TTTACCAGTTGACATAGCATATAAGAGGGGCTA |
| | | TTTCAATGAGGAACTCAGTGAGATTCTCTCAGA |
| | | TCCAAGTGATGATACCAAAGGATTTTTTGACCC |
| | | CAACACTGAAGAAAATCTTACCTATCTGCAACT |
| | | AAAAGAAAGATGCATTAAGGATGAGGAAACAG |
| | | GGCTCTGTCTTCTGCCTCTGAAAGAAAAGAAGA |
| | | AACAGGTGCAGACATCACAAAAGAATACCCTC |
| | | AGGAAGCGTAGAGTGGTCATAGTTGACCCAGA |
| | | AACCAATAAAGAAATGTCTGTTCAGGAGGCCT |
| | | ACAAGAAGGGCCTAATTGATTATGAAACCTTC |
| | | AAAGAACTGTGTGAGCAGGAATGTGAATGGGA |
| | | AGAAATAACCATCACGGGATCAGATGGCTCCA |
| | | CCAGGGTGGTCCTGGTAGATAGAAAGACAGGC |
| | | AGTCAGTATGATATTCAAGATGCTATTGACAAG |
| | | GGCCTTGTTGACAGGAAGTTCTTTGATCAGTAC |
| | | CGATCCGGCAGCCTCAGCCTCACTCAATTTGCT |
| | | GACATGATCTCCTTGAAAAATGGTGTCGGCACC |
| | | AGCAGCAGCATGGGCAGTGGTGTCAGCGATGA |
| | | TGTTTTTAGCAGCTCCCGACATGAATCAGTAAG |
| | | TAAGATTTCCACCATATCCAGCGTCAGGAATTT |
| | | AACCATAAGGAGCAGCTCTTTTTCAGACACCCT |
| | | GGAAGAATCGAGCCCCATTGCAGCCATCTTTGA |
| | | CACAGAAAACCTGGAGAAAATCTCCATTACAG |
| | | AAGGTATAGAGCGGGGCATCGTTGACAGCATC |
| | | ACGGGTCAGAGGCTT |
| | | CTGGAGGCTCAGGCCTGCACAGGTGGCATCAT |
| | | CCACCCAACCACGGGCCAGAAGCTGTCACTTC |
| | | AGGACGCAGTCTCCCAGGGTGTGATTGACCAA |
| | | GACATGGCCACCAGGCTGAAGCCTGCTCAGAA |
| | | AGCCTTCATAGGCTTCGAGGGTGTGAAGGGAA |
| | | AGAAGAAGATGTCAGCAGCAGAGGCAGTGAAA |
| | | GAAAAATGGCTCCCGTATGAGGCTGGCCAGCG |
| | | CTTCCTGGAGTTCCAGTACCTCACGGGAGGTCT |
| | | TGTTGACCCGGAAGTGCATGGGAGGATAAGCA |
| | | CCGAAGAAGCCATCCGGAAGGGGTTCATAGAT |
| | | GGCCGCGCCGCACAGAGGCTGCAAGACACCAG |
| | | CAGCTATGCCAAAATCCTGACCTGCCCCAAAAC |
| | | CAAATTAAAAATATCCTATAAGGATGCCATAA |
| | | ATCGCTCCATGGTAGAAGATATCACTGGGCTGC |
| | | GCCTTCTGGAAGCCGCCTCCGTGTCGTCCAAGG |
| | | GCTTACCCAGCCCTTACAACATGTCTTCGGCTC |
| | | CGGGGTCCCGCTCCGGCTCCCGCTCGGGATCTC |
| | | GCTCCGGATCTCGCTCCGGGTCCCGCAGTGGGT |
| | | CCCGGAGAGGAAGCTTTGACGCCACAGGGAAT |
| | | TCTTCCTACTCTTATTCCTACTCATTTAGCAGTA |
| | | GTTCTATTGGGCACTAG (SEQ ID NO: 219) |
| Human DSP | MSCNGGSHPRINTLGRMIRAESGP | ATGAGCTGCAACGGAGGCTCCCACCCGCGGAT |
| DPII | DLRYEVTSGGGGTSRMYYSRRG | CAACACTCTGGGCCGCATGATCCGCGCCGAGTC |
| isoform | VITDQNSDGYCQTGTMSRHQNQ | TGGCCCGGACCTGCGCTACGAGGTGACCAGCG |
| | NTIQELLQNCSDCLMRAELIVQPE | GCGGCGGGGGCACCAGCAGGATGTACTATTCT |
| | LKYGDGIQLTRSRELDECFAQAN | CGGCGCGGCGTGATCACCGACCAGAACTCGGA |
| | DQMEILDSLIREMRQMGQPCDAY | CGGCTACTGTCAAACCGGCACGATGTCCAGGC |
| | QKRLLQLQEQMRALYKAISVPRV | ACCAGAACCAGAACACCATCCAGGAGCTGCTG |
| | RRASSKGGGGYTCQSGSGWDEFT | CAGAACTGCTCCGACTGCTTGATGCGAGCAGA |
| | KHVTSECLGWMRQQRAEMDMV | GCTCATCGTGCAGCCTGAATTGAAGTATGGAG |
| | AWGVDLASVEQHINSHRGIHNSI | ATGGAATACAACTGACTCGGAGTCGAGAATTG |
| | GDYRWQLDKIKADLREKSAIYQL | GATGAGTGTTTTGCCCAGGCCAATGACCAAATG |
| | EEEYENLLKASFERMDHLRQLQN | GAAATCCTCGACAGCTTGATCAGAGAGATGCG |
| | IIQATSREIMWINDCEEEELLYDW | GCAGATGGGCCAGCCCTGTGATGCTTACCAGA |
| | SDKNTNIAQKQEAFSIRMSQLEVK | AAAGGCTTCTTCAGCTCCAAGAGCAAATGCGA |
| | EKELNKLKQESDQLVLNQHPASD | GCCCTTTATAAAGCCATCAGTGTCCCTCGAGTC |
| | KIEAYMDTLQTQWSW | CGCAGGGCCAGCTCCAAGGGTGGTGGAGGCTA |
| | ILQITKCIDVHLKENAAYFQFFEE | CACTTGTCAGAGTGGCTCTGGCTGGGATGAGTT |
| | AQSTEAYLKGLQDSIRKKYPCDK | CACCAAACATGTCACCAGTGAATGTTTGGGGTG |
| | NMPLQHLLEQIKELEKEREKILEY | GATGAGGCAGCAAAGGGCGGAGATGGACATGG |
| | KRQVQNLVNKSKKIVQLKPRNPD | TGGCCTGGGGTGTGGACCTGGCCTCAGTGGAG |
| | YRSNKPIILRALCDYKQDQKIVHK | CAGCACATTAACAGCCACCGGGGCATCCACAA |
| | GDECILKDNNERSKWYVTGPGGV | CTCCATCGGCGACTATCGCTGGC |
| | DMLVPSVGLIIPPPNPLAVDLSCKI | AGCTGGACAAAATCAAAGCCGACCTGCGCGAG |
| | EQYYEAILALWNQLYINMKSLVS | AAATCTGCGATCTACCAGTTGGAGGAGGAGTA |
| | WHYCMIDIEKIRAMTIAKLKTMR | TGAAAACCTGCTGAAAGCGTCCTTTGAGAGGA |
| | QEDYMKTIADLELHYQEFIRNSQ | TGGATCACCTGCGACAGCTGCAGAACATCATTC |
| | GSEMFGDDDKRKIQSQFTDAQKH | AGGCCACGTCCAGGGAGATCATGTGGATCAAT |
| | YQTLVIQLPGYPQHQTVTTTEITH | GACTGCGAGGAGGAGGAGCTGCTGTACGACTG |
| | HGTCQDVNHNKVIETNRENDKQE | GAGCGACAAGAACACCAACATCGCTCAGAAAC |
| | TWMLMELQKIRRQIEHCEGRMTL | AGGAGGCCTTCTCCATACGCATGAGTCAACTGG |
| | KNLPLADQGSSHHITVKINELKSV | AAGTTAAAGAAAAAGAGCTCAATAAGCTGAAA |
| | QNDSQAIAEVLNQLKDMLANFRG | CAAGAAAGTGACCAACTTGTCCTCAATCAGCAT |
| | SEKYCYLQNEVFGLFQKLENING | CCAGCTTCAGACAAAATTGAGGCCTATATGGA |
| | VTDGYLNSLCTVRALLQAILQTE | CACTCTGCAGACGCAGTGGAGTTGGATTCTTCA |
| | DMLKVYEARLTEEETVCLDLDKV | GATCACCAAGTGCATTGATGTTCATCTGAAAGA |
| | EAYRCGLKKIKNDLNLKKSLLAT | AAATGCTGCCTACTTTCAGTTTTTTGAAGAGGC |
| | MKTELQKAQQIHS | GCAGTCTACTGAAGCATACCTGAAGGGGCTCC |
| | QTSQQYPLYDLDLGKFGEKVTQL | AGGACTCCATCAGGAAGAAGTACCCCTGCGAC |
| | TDRWQRIDKQIDFRLWDLEKQIK | AAGAACATGCCCCTGCAGCACCTGCTGGAACA |
| | QLRNYRDNYQAFCKWLYDAKRR | GATCAAGGAGCTGGAGAAAGAACGAGAGAAA |
| | QDSLESMKFGDSNTVMRFLNEQK | ATCCTTGAATACAAGCGTCAGGTGCAGAACTTG |
| | NLHSEISGKRDKSEEVQKIAELCA | GTAAACAAGTCTAAGAAGATTGTACAGCTGAA |
| | NSIKDYELQLASYTSGLETLLNIPI | GCCTCGTAACCCAGACTACAGAAGCAATAAAC |
| | KRTMIQSPSGVILQEAADVHARYI | CCATTATTCTCAGAGCTCTCTG |
| | ELLTRSGDYYRELSEMLKSLEDL | TGACTACAAACAAGATCAGAAAATCGTGCATA |
| | KLKNTKIEVLEEELRLARDANSEN | AGGGGGATGAGTGTATCCTGAAGGACAACAAC |
| | CNKNKFLDQNLQKYQAECSQFK | GAGCGCAGCAAGTGGTACGTGACGGGCCCGGG |
| | AKLASLEELKRQAELDGKSAKQN | AGGCGTTGACATGCTTGTTCCCTCTGTGGGGCT |
| | LDKCYGQIKELNEKITRLTYEIED | GATCATCCCTCCTCCGAACCCACTGGCCGTGGA |
| | EKRRRKSVEDRFDQQKNDYDQL | CCTCTCTTGCAAGATTGAGCAGTACTACGAAGC |
| | QKARQCEKENLGWQKLESEKAIK | CATCTTGGCTCTGTGGAACCAGCTCTACATCAA |
| | EKEYEIERLRVLLQEEGTRKREYE | CATGAAGAGCCTGGTGTCCTGGCACTACTGCAT |
| | NELAKASNRIQESKNQCTQVVQE | GATTGACATAGAGAAGATCAGGGCCATGACAA |
| | RESLLVKIKVLEQDKARLQRLED | TCGCCAAGCTGAAAACAATGCGGCAGGAAGAT |
| | ELNRAKSTLEAETRVKQRLECEKQQ | TACATGAAGACGATAGCCGACCTTGAGTTACAT |
| | IQNDLNQWKTQYSRKEEAIRKIES | TACCAAGAGTTCATCAGAAATAGCCAAGGCTC |
| | EREKSEREKNSLRSEIERLQAEIKR | AGAGATGTTTGGAGATGATGACAAGCGGAAAA |
| | IEERCRRKLED | TACAGTCTCAGTTCACCGATGCCCAGAAGCATT |
| | STRETQSQLETERSRYQREIDKLR | ACCAGACCCTGGTCATTCAGCTCCCTGGCTATC |
| | QRPYGSHRETQTECEWTVDTSKL | CCCAGCACCAGACAGTGACCACAACTGAAATC |
| | VFDGLRKKVTAMQLYECQLIDKT | ACTCATCATGGAACCTGCCAAGATGTCAACCAT |
| | TLDKLLKGKKSVEEVASEIQPFLR | AATAAAGTAATTGAAACCAACAGAGAAAATGA |
| | GAGSIAGASASPKEKYSLVEAKR | CAAGCAAGAAACATGGATGCTGATGGAGCTGC |
| | KKLISPESTVMLLEAQAATGGIID | AGAAGATTCGCAGGCAGATAGAGCACTGCGAG |
| | PHRNEKLTVDSAIARDLIDFDDRQ | GGCAGGATGACTCTCAAAAACCTCCCTCTAGCA |
| | QIYAAEKAITGFDDPFSGKTVSVS | GACCAGGGATCTTCTCACCACATCACAGTGAA |
| | EAIKKNLIDRETGMRLLEAQIASG | AATTAACGAGCTTAAGAGTGTGCAGAATGATT |
| | GVVDPVNSVFLPKDVALARGLID | CACAAGCAATTGCTGAGGTTCTCAACCAGCTTA |
| | RDLYRSLNDPRDSQKNFVDPVTK | AAGATATGCTTGCCAACTTCAGAGGTTCTGAAA |
| | KKVSYVQLKERVRIEPHTGLLLLS | AGTACTGCTATTTACAGAATGAAGTATTTGGAC |
| | VQKRSMSFQGIRQPVTVTELVDS | TATTTCAGAAACTGGAAAATATCAATGGTGTTA |
| | GILRPSTVNELESGQISYDEVGERI | CAGATGGCTACTTAAATAGCTTATGCACAGTAA |
| | KDFLQGSSCIAGIYNETTKQKLGI | GGGCACTGCTCCAGGCTATTCTCCAAACAGAA |
| | YEAMKIGLVRPGTALELLEAQAA | GACATGTTAAAGGTTTATGAAGCCAGGCTCACT |
| | TGFIVDPVSNLRLPVEEAYKRGLV | GAGGAGGAAACTGTCTGCCTGGACCTGGATAA |
| | GIEFKEKLLSAERAVTGYNDPETG | AGTGGAAGCTTACCGCTGTGGACTGAAGAAAA |
| | NIISLFQAMNKELIEKGHGIRLLEA | TAAAAAATGA (SEQ ID NO: 221) |
| | QIATGGIIDPKESHRLPVDIAYKRG | CTTGAACTTGAAGAAGTCGTTGTTGGCCACTAT |
| | YFNEELSEILSDPSDDTKGFFDPNT | GAAGACAGAACTACAGAAAGCCCAGCAGATCC |
| | EENLTYLQLKERCIKDEETGLCLL | ACTCTCAGACTTCACAGCAGTATCCACTTTATG |
| | PLKEKKKQVQTSQKNTLRKRRVV | ATCTGGACTTGGGCAAGTTCGGTGAAAAAGTC |
| | IVDPETNKEMSVQEAYKKGLIDY | ACACAGCTGACAGACCGCTGGCAAAGGATAGA |
| | ETFKELCEQECEWEEITITGSDGST | TAAACAGATCGACTTTAGGTTATGGGACCTGGA |
| | RVVLVDRKTGSQYDIQDAIDKGL | GAAACAAATCAAGCAATTGAGGAATTATCGTG |
| | VDRKFFDQYRSGSLSLTQFADMIS | ATAACTATCAGGCTTTCTGCAAGTGGCTCTATG |
| | LKNGVGTSSSMGSGVSDDVFSSS | ATGCTAAACGCCGCCAGGATTCCTTAGAATCCA |
| | RHESVSKISTISSVRNLTIRSSSFSD | TGAAATTTGGAGATTCCAACACAGTCATGCGGT |
| | TLEESSPIAAIFDTENLEKISITEGIE | TTTTGAATGAGCAGAAGAACTTGC |
| | RGIVDSITGQRLLEAQACTGGIIHP | ACAGTGAAATATCTGGCAAACGAGACAAATCA |
| | TTGQKLSLQDAVSQGVIDQDMAT | GAGGAAGTACAAAAAATTGCTGAACTTTGCGC |
| | RLKPAQKAFIGFEGVKGKKKMSA | CAATTCAATTAAGGATTATGAGCTCCAGCTGGC |
| | AEAVKEKWLPYEAGQRFLEFQYL | CTCATACACCTCAGGACTGGAAACTCTGCTGAA |
| | TGGLVDPEVHGRISTEEAIRKGFI | CATACCTATCAAGAGGACCATGATTCAGTCCCC |
| | DGRAAQRLQDTSSAKILTCPKT | TTCTGGGGTGATTCTGCAAGAGGCTGCAGATGT |
| | KLKISYKDAINRSMVEDITGLRLL | TCATGCTCGGTACATTGAACTACTTACAAGATC |
| | EAASVSSKGLPSPYNMSSAPGSRS | TGGAGACTATTACAGGTTCTTAAGTGAGATGCT |
| | GSRSGSRSGSRSGSRSGSRRGSFD | GAAGAGTTTGGAAGATCTGAAGCTGAAAAATA |
| | ATGNSSYSYSYSFSSSSIGH | CCAAGATCGAAGTTTTGGAAGAGGAGCTCAGA |
| | (SEQ ID NO: 222) | CTGGCCCGAGATGCCAACTCGGAA |
| | | AACTGTAATAAGAACAAATTCCTGGATCAGAA |
| | | CCTGCAGAAATACCAGGCAGAGTGTTCCCAGTT |
| | | CAAAGCGAAGCTTGCGAGCCTGGAGGAGCTGA |
| | | AGAGACAGGCTGAGCTGGATGGGAAGTCGGCT |
| | | AAGCAAAATCTAGACAAGTGCTACGGCCAAAT |
| | | AAAAGAACTCAATGAGAAGATCACCCGACTGA |
| | | CTTATGAGATTGAAGATGAAAAGAGAAGAAGA |
| | | AAATCTGTGGAAGACAGATTTGACCAACAGAA |
| | | GAATGACTATGACCAACTGCAGAAAGCAAGGC |
| | | AATGTGAAAAGGAGAACCTTGGTTGGCAGAAA |
| | | TTAGAGTCTGAGAAAGCCATCAAGGAGAAGGA |
| | | GTACGAGATTGAAAGGTTGAGGGTTCTACTGC |
| | | AGGAAGAAGGCACCC |
| | | GGAAGAGAGAATATGAAAATGAGCTGGCAAAG |
| | | GCATCTAATAGGATTCAGGAATCAAAGAATCA |
| | | GTGTACTCAGGTGGTACAGGAAAGAGAGAGCC |
| | | TTCTGGTGAAAATCAAAGTCCTGGAGCAAGAC |
| | | AAGGCAAGGCTGCAGAGGCTGGAGGATGAGCT |
| | | GAATCGTGCAAAATCAACTCTAGAGGCAGAAA |
| | | CCAGGGTGAAACAGCGCCTGGAGTGTGAGAAA |
| | | CAGCAAATTCAGAATGACCTGAATCAGTGGAA |
| | | GACTCAATATTCCCGCAAGGAGGAGGCTATTA |
| | | GGAAGATAGAATCGGAAAGAGAAAAGAGTGA |
| | | GAGAGAGAAGAACAGTCTTAGGAGTGAGATCG |
| | | AAAGACTCCAAGCAGAGATCAAGAGAATTGAA |
| | | GAGAGGTGCAGGCGTAAGCTGGAGGATTCTAC |
| | | CAGGGAGACACAGTCACAGTTAGAAACAGAACGC |
| | | TCCCGATATCAGAGGGAGATTGATAAACTCAGA |
| | | CAGCGCCCATATGGGTCCCATCGAGAGACCCA |
| | | GACTGAGTGTGAGTGGACCGTTGACACCTCCA |
| | | AGCTGGTGTTTGATGGGCTGAGGAAGAAGGTG |
| | | ACAGCAATGCAGCTCTATGAGTGTCAGCTGATC |
| | | GACAAAACAACCTTGGACAAACTATTGAAGGG |
| | | GAAGAAGTCAGTGGAAGAAGTTGCTTCTGAAA |
| | | TCCAGCCATTCCTTCGGGGTGCAGGATCTATCG |
| | | CTGGAGCATCTGCTTCTCCTAAGGAAAAATACT |
| | | CTTTGGTAGAGGCCAAGAGAAAGAAATTAATC |
| | | AGCCCAGAATCCACAGTCATGCTTCTGGAGGCC |
| | | CAGGCAGCTACAGGTGGTATAATTGATCCCCAT |
| | | CGGAATGAGAAGCTGACTGTCGACAGTGCCAT |
| | | AGCTCGGGACCTCATTGACTTCGATGACCGTCA |
| | | GCAGATATATGCAGCAGAAAAAGCTATCACTG |
| | | GTTTTGATGATCCATTTTCAGGCAAGACAGTAT |
| | | CTGTTTCAGAAGCCATCAAGAAAAATTTGATTG |
| | | ATAGAGAAACCGGAATGCGCCTGCTGGAAGCC |
| | | CAGATTGCTTCAGGGGGTGTAGTAGACCCTGTG |
| | | AACAGTGTCTTTTTGCCAAAAGATGTCGCCTTG |
| | | GCCCGGGGGCTGATTGATAGAGATTTGTATCGA |
| | | TCCCTGAATGATCCCCGAGATAGTCAGAAAAA |
| | | CTTTGTGGATCCAGTCACCAAAAAGAAGGTCA |
| | | GTTACGTGCAGCTGAAGGAACGGTGCAGAATC |
| | | GAACCACATACTGGTCTGCTCTTGCTTTCAGTA |
| | | CAGAAGAGAAGCATGTCCTTCCAAGGAATCAG |
| | | ACAACCTGTGACCGTCACTGAGCTAGTAGATTC |
| | | TGGTATATTGAGACCGTCCACTGTCAATGAACT |
| | | GGAATCTGGTCAGATTTCTTATGACGAGGTTGG |
| | | TGAGAGAATTAAGGACTTCCTCCAGGGTTCAA |
| | | GCTGCATAGCAGGCATATACAATGAGACCACA |
| | | AAACAGAAGCTTGGCATTTATGAGGCCATGAA |
| | | AATTGGCTTAGTCCGACCTGGTACTGCTCTGGA |
| | | GTTGCTGGAAGCCCAAGCAGCTACTGGCTTTAT |
| | | AGTGGATCCTGTTAGCAACTTGAGGTTACCAGT |
| | | GGAGGAAGCCTACAAGAGAGGTCTGGTGGGCA |
| | | TTGAGTTCAAAGAGAAGCTCCTGTCTGCAGAAC |
| | | GAGCTGTCACTGGGTATAATGATCCTGAAACA |
| | | GGAAACATCATCTCTTTGTTCCAAGCCATGAAT |
| | | AAGGAACTCATCGAAAAGGGCCACGGTATTCG |
| | | CTTATTAGAAGCACAGATCGCAACCGGGGGGA |
| | | TCATTGACCCAAAGGAGAGCCATCGTTTACCAG |
| | | TTGACATAGCATATAAGAGGGGCTATTTCAATGA |
| | | GGAACTCAGTGAGATTCTCTCAGATCCAAGTGAT |
| | | GATACCAAAGGATTTTTTGACCCCAACACTGAA |
| | | GAAAATCTTACCTATCTGCAACTAAAAGAAAG |
| | | ATGCATTAAGGATGAGGAAACAGGGCTCTGTC |
| | | TTCTGCCTCTGAAAGAAAAGAAGAAACAGGTG |
| | | CAGACATCACAAAAGAATACCCTCAGGAAGCG |
| | | TAGAGTGGTCATAGTTGACCCAGAAACCAATA |
| | | AAGAAATGTCTGTTCAGGAGGCCTACAAGAAG |
| | | GGCCTAATTGATTATGAAACCTTCAAAGAACTG |
| | | TGTGAGCAGGAATGTGAATGGGAAGAAATAAC |
| | | CATCACGGGATCAGATGGCTCCACCAGGGTGG |
| | | TCCTGGTAGATAGAAAGACAGGCAGTCAGTAT |
| | | GATATTCAAGATGCTATTGACAAGGGCCTTGTT |
| | | GACAGGAAGTTCTTTGATCAGTACCGATCCGGC |
| | | AGCCTCAGCCTCACTCAATTTGCTGACATGATC |
| | | TCCTTGAAAAATGGTGTCGGCACCAGCAGCAG |
| | | CATGGGCAGTGGTGTCAGCGATGATGTTTTTAG |
| | | CAGCTCCCGACATGAATCAGTAAGTAAGATTTC |
| | | CACCATATCCAGCGTCAGGAATTTAACCATAAG |
| | | GAGCAGCTCTTTTTCAGACACCCTGGAAGAATC |
| | | GAGCCCCATTGCAGCCATCTTTGACACAGAAA |
| | | ACCTGGAGAAAATCTCCA |
| | | TTACAGAAGGTATAGAGCGGGGCATCGTTGAC |
| | | AGCATCACGGGTCAGAGGCTTCTGGAGGCTCA |
| | | GGCCTGCACAGGTGGCATCATCCACCCAACCA |
| | | CGGGCCAGAAGCTGTCACTTCAGGACGCAGTC |
| | | TCCCAGGGTGTGATTGACCAAGACATGGCCAC |
| | | CAGGCTGAAGCCTGCTCAGAAAGCCTTCATAG |
| | | GCTTCGAGGGTGTGAAGGGAAAGAAGAAGATG |
| | | TCAGCAGCAGAGGCAGTGAAAGAAAAATGGCT |
| | | CCCGTATGAGGCTGGCCAGCGCTTCCTGGAGTT |
| | | CCAGTACCTCACGGGAGGTCTTGTTGACCCGGA |
| | | AGTGCATGGGAGGATAAGCACCGAAGAAGCCA |
| | | TCCGGAAGGGGTTCATAGATGGCCGCGCCGCA |
| | | CAGAGGCTGCAAGACACCAGCAGCTATGCCAA |
| | | AATCCTGACCTGCCCCAAAACCAAATTAAAAA |
| | | TATCCTATAAGGATGCCATAAATCGCTCCATGG |
| | | TAGAAGATATCACTGGGCTGCGCCTTCTGGAAG |
| | | CCGCCTCCGTGTCGTCCAAGGGCTTACCCAGCC |
| | | CTTACAACATGTCTTCGGCTCCGGGGTCCCGCT |
| | | CCGGCTCCCGCTCGGGATCTCGCTCCGGATCTC |
| | | GCTCCGGGTCCCGCAGTGGGTCCCGGAGAGGA |
| | | AGCTTTGACGCCACAGGGAATTCTTCCTACTCT |
| | | TATTCCTACTCATTTAGCAGTAGTTCTATTGGG |
| | | CACTAG (SEQ ID NO: 221) |
| Human | MARSPGRAYALLLLLICFNVGSG | ATGGCGCGGAGCCCGGGACGCGCGTACGCCCT |
| DSG2 | LHLQVLSTRNENKLLPKHPHLVR | GCTGCTTCTCCTGATCTGCTTTAACGTTGGAAG |
| | QKRAWITAPVALREGEDLSKKNP | TGGACTTCACTTACAGGTCTTAAGCACAAGAAA |
| | IAKIHSDLAEERGLKITYKYTGKG | TGAAAATAAGCTGCTTCCTAAACATCCTCATTT |
| | ITEPPFGIFVFNKDTGELNVTSILD | AGTGCGGCAAAAGCGCGCCTGGATCACCGCCC |
| | REETPFFLLTGYALDARGNNVEK | CCGTGGCTCTTCGGGAGGGAGAGGATCTGTCC |
| | PLELRIKVLDINDNEPVFTQDVFV | AAGAAGAATCCAATTGCCAAGATACATTCTGA |
| | GSVEELSAAHTLVMKINATDADE | TCTTGCAGAAGAAAGAGGACTCAAAATTACTT |
| | PNTLNSKISYRIVSLEPAYPPVFYL | ACAAATACACTGGAAAAGGGATTACAGAGCCA |
| | NKDTGEIYTTSVTLDREEHSSYTL | CCTTTTGGTATATTTGTCTTTAACAAAGATACT |
| | TVEARDGNGEVTDKPVKQAQVQI | GGAGAACTGAATGTTACCAGCATTCTTGATCGA |
| | RILDVNDNIPVVENKVLEGMVEE | GAAGAAACACCATTTTTTCTGCTAACAGGTTAC |
| | NQVNVEVTRIKVFDADEIGSDNW | GCTTTGGATGCAAGAGGAAACAATGTAGAGAA |
| | LANFTFASGNEGGYFHIETDAQT | ACCCTTAGAGCTACGCATTAAGGTTCTTGATAT |
| | NEGIVTLIKEVDYEEMKNLDFSVI | CAATGACAACGAACCAGTGTTCACACAGGATG |
| | VANKAAFHKSIRSKYKPTPIPIKV | TCTTTGTTGGGTCTGTTGAAGAGTTGAGTGCAG |
| | KVKNVKEGIHFKSSVISIYVSESM | CACATACTCTTGTGATGAAAATCAATGCAACAG |
| | DRSSKGQIIGNFQAFDEDTGLPAH | ATGCAGATGAGCCCAATACCCTGAATTCGAAA |
| | ARYVKLEDRDNWISVDSVTSEIK | ATTTCCTATAGAATCGTATCTCTGGAGCCTGCT |
| | LAKLPDFESRYVQNGTYTVKIVAI | TATCCTCCAGTGTTCTACCTAAATAAAGATACA |
| | SEDYPRKTITGTVLINVEDINDNC | GGAGAGATTTATACAACCAGTGTTACCTTGGAC |
| | PTLIEPVQTICHDAEYVNVTAEDL | AGAGAGGAACACAGCAGCTACACTTTGACAGT |
| | DGHPNSGPFSFSVIDKPPGMAEK | AGAAGCAAGAGATGGCAATGGAGAAGTTACAG |
| | WKIARQESTSVLLQQSEKKLGRS | ACAAACCTGTAAAACAAGCTCAAGTTCAGATT |
| | EIQFLISDNQGFSCPEKQVLTLTVC | CGTATTTTGGATGTCAATGACAATATACCTGTA |
| | ECLHGSGCREAQHDSYVGLGPAA | GTAGAAAATAAAGTGCTTGAAGGGATGGTTGA |
| | IALMILAFLLLLLVPLLLLMCHCG | AGAAAATCAAGTCAACGTAGAAGTTACGCGCA |
| | KGAKGFTPIPGTIEMLHPWNNEG | TAAAAGTGTTCGATGCAGATGAAATAGGTTCTG |
| | APPEDKVVPSFLPVDQGGSLVGR | ATAATTGGCTGGCAAATTTTACATTTGCATCAG |
| | NGVGGMAKEATMKGSSSASIVK | GAAATGA |
| | GQHEMSEMDGRWEEHRSLLSGR | AGGAGGTTATTTCCACATAGAAACAGATGCTC |
| | ATQFTGATGAIMTTETTKTARAT | AAACTAACGAAGGAATTGTGACCCTTATTAAG |
| | GASRDMAGAQAAAVALNEEFLR | GAAGTAGATTATGAAGAAATGAAGAATCTTGA |
| | NYFTDKAASYTEEDENHTAKDCL | CTTCAGTGTTATTGTCGCTAATAAAGCAGCTTT |
| | LVYSQEETESLNASIGCCSFIEGEL | TCACAAGTCGATTAGGAGTAAATACAAGCCTA |
| | DDRFLDDLGLKFKTLAEVCLGQK | CACCCATTCCCATCAAGGTCAAAGTGAAAAAT |
| | IDINKEIEQRQKPATETSMNTASH | GTGAAAGAAGGCATTCATTTTAAAAGCAGCGT |
| | SLCEQTMVNSENTYSSGSSFPVPK | CATCTCAATTTATGTTAGCGAGAGCATGGATAG |
| | SLQEANAEKVTQEIVTERSVSSRQ | ATCAAGCAAAGGCCAAATAATTGGAAATTTTC |
| | AQKVATPLPDPMASRNVIATETS | AAGCTTTTGATGAGGACACTGGACTACCAGCCC |
| | YVTGSTMPPTTVILGPSQPQSLIVT | ATGCAAGATATGTAAAATTAGAAGATAGAGAT |
| | ERVYAPASTLVDQPYANEGTVVV | AATTGGATCTCTGTGGATTCTGTCACATCTGAA |
| | TERVIQPHGGGSNPLEGTQHLQD | ATTAAACTTGCAAAACTTCCTGATTTTGAATCT |
| | VPYVMVRERESFLAPSSGVQPTL | AGATATGTTCAAAATGGCACATACACTGTAAA |
| | AMPNIAVGQNVTVTERVLAPAST | GATTGTGGCCATATCAGAAGATTATCCTAGAAA |
| | LQSSYQIPTENSMTARNTTVSGAG | AACCATCACTGGCACAGTCCTTATCAATGTTGA |
| | VPGPLPDFGLEESGHSNSTITTSST | AGACATCAACGACAACTGTCCCACACTGATAG |
| | RVTKHSTVQHSYS | AGCCTGTGCAGACAATCTGTCACGATGCAGAG |
| | (SEQ ID NO: 224) | TATGTGAATGTTACTGCAGAGGACCTGGATGG |
| | | ACACCCAAACAGTGGCCCTTTCAGTTTCTCCGT |
| | | CATTGACAAACCACCTGGCATGGCAGAAAAAT |
| | | GGAAAATAGCACGCCAAGAAAGTACCAGTGTG |
| | | CTGCTGCAACAAAGTGAGAAAAAGCTTGGGAG |
| | | AAGTGAAATTCAGTTCCTGATTTCAGACAATCA |
| | | GGGTTTTAGTTGTCCTGAAAAGCAGGTCCTTAC |
| | | ACTCACAGTTTGTGAGTGTCTGCATGGCAGCGG |
| | | CTGCAGGGAAGCACAGCATGACTCCTATGTGG |
| | | GCCTGGGACCCGCAGCAATTGCGCTCATGATTT |
| | | TGGCCTTTCTGCTCCTGCTATTGGTACCACTTTT |
| | | ACTGCTGA |
| | | TGTGCCATTGCGGAAAGGGCGCCAAAGGCTTT |
| | | ACCCCCATACCTGGCACCATAGAGATGCTGCAT |
| | | CCTTGGAATAATGAAGGAGCACCACCTGAAGA |
| | | CAAGGTGGTGCCATCATTTCTGCCAGTGGATCA |
| | | AGGGGGCAGTCTAGTAGGAAGAAATGGAGTAG |
| | | GAGGTATGGCCAAGGAAGCCACGATGAAAGGA |
| | | AGTAGCTCTGCTTCCATTGTCAAAGGGCAACAT |
| | | GAGATGTCCGAGATGGATGGAAGGTGGGAAGA |
| | | ACACAGAAGCCTGCTTTCTGGTAGAGCTACCCA |
| | | GTTTACAGGGGCCACAGGCGCTATCATGACCA |
| | | CTGAAACCACGAAGACCGCAAGGGCCACAGGG |
| | | GCTTCCAGAGACATGGCCGGAGCTCAGGCAGC |
| | | TGCTGTTGCACTGAACGAAGAATTCTTAAGAAA |
| | | TTATTTCACTGATAAAGCGGCCTCTTACACTGA |
| | | GGAAGATGAAAATCACACAGCCAAAGATTGCC |
| | | TTCTGGTTTATTCTCAGGAAGAAACTGAATCGC |
| | | TGAATGCTTCTATTGGTTGTTGCAGTTTTATTGA |
| | | AGGAGAGCTAGATGACCGCTTCTTAGATGATTT |
| | | GGGACTTAAATTCAAGACACTAGCTGAAGTTTG |
| | | CCTGGGTCAAAAAATAGATATAAATAAGGAAA |
| | | TTGAGCAGAGACAAAAACCTGCCACAGAAACA |
| | | AGTATGAACACAGCTTC |
| | | ACATTCACTCTGTGAGCAAACTATGGTTAATTC |
| | | AGAGAATACCTACTCCTCTGGCAGTAGCTTCCC |
| | | AGTTCCAAAATCTTTGCAAGAAGCCAATGCAG |
| | | AGAAAGTAACTCAGGAAATAGTCACTGAAAGA |
| | | TCTGTGTCTTCTAGGCAGGCGCAAAAGGTAGCT |
| | | ACACCTCTTCCTGACCCAATGGCTTCTAGAAAT |
| | | GTGATAGCAACAGAAACTTCCTATGTCACAGG |
| | | GTCCACTATGCCACCAACCACTGTGATCCTGGG |
| | | TCCTAGCCAGCCACAGAGCCTTATTGTGACAGA |
| | | GAGGGTGTATGCTCCAGCTTCTACCTTGGTAGA |
| | | TCAGCCTTATGCTAATGAAGGTACAGTTGTGGT |
| | | CACTGAAAGAGTAATACAGCCTCATGGGGGTG |
| | | GATCGAATCCTCTGGAAGGCACTCAGCATCTTC |
| | | AAGATGTACCTTACGTCATGGTGAGGGAAAGA |
| | | GAGAGCTTCCTTGCCCCCAGCTCAGGTGTGCAG |
| | | CCTACTCTGGCCATGCCTAATATAGCAGTAGGA |
| | | CAGAATGTGACAGTGACAGAAAGAGTTCTAGC |
| | | ACCTGCTTCCACTCTGCAATCCAGTTACCAGAT |
| | | TCCCACTGAAAATTCTATGACGGCTAGGAACAC |
| | | CACGGTGTCTGGAGCTGGAGTCCCTGGCC |
| | | CTCTGCCAGATTTTGGTTTAGAGGAATCTGGTC |
| | | ATTCTAATTCTACCATAACCACATCTTCCACCA |
| | | GAGTTACCAAGCATAGCACTGTACAGCATTCTT |
| | | ACTCCTAA (SEQ ID NO: 223) |
| Human JUP | MEVMNLMEQPIKVTEWQQTYTY | ATGGAGGTGATGAACCTGATGGAGCAGCCTAT |
| | DSGIHSGANTCVPSVSSKGIMEED | CAAGGTGACTGAGTGGCAGCAGACATACACCT |
| | EACGRQYTLKKTTTYTQGVPPSQ | ACGACTCGGGTATCCACTCGGGCGCCAACACCT |
| | GDLEYQMSTTARAKRVREAMCP | GCGTGCCCTCCGTCAGCAGCAAGGGCATCATG |
| | GVSGEDSSLLLATQVEGQATNLQ | GAGGAGGATGAGGCCTGCGGGCGCCAGTACAC |
| | RLAEPSQLLKSAIVHLINYQDDAE | GCTCAAGAAAACCACCACTTACACCCAGGGGG |
| | LATRALPELTKLLNDEDPVVVTK | TGCCCCCCAGCCAAGGTGATCTGGAGTACCAG |
| | AAMIVNQLSKKEASRRALMGSPQ | ATGTCCACAACAGCCAGGGCCAAACGGGTGCG |
| | LVAAVVRTMQNTSDLDTARCTTS | GGAGGCCATGTGCCCTGGTGTGTCAGGCGAGG |
| | ILHNLSHHREGLLAIFKSGGIPALV | ACAGCTCGCTTCTGCTGGCCACCCAGGTGGAGG |
| | RMLSSPVESVLFYAITTLHNLLLY | GGCAGGCCACCAACCTGCAGCGACTGGCCGAG |
| | QEGAKMAVRLADGLQKMVPLLN | CCGTCCCAGCTGCTCAAGTCGGCCATTGTGCAT |
| | KNNPKFLAITTDCLQLLAYGNQE | CTCATCAACTACC |
| | SKLIILANGGPQALVQIMRNYSYE | AGGACGATGCCGAGCTGGCCACTCGCGCCCTG |
| | KLLWTTSRVLKVLSVCPSNKPAIV | CCCGAGCTCACCAAACTGCTCAACGACGAGGA |
| | EAGGMQALGKHLTSNSPRLVQN | CCCGGTGGTGGTGACCAAGGCGGCCATGATTG |
| | CLWTLRNLSDVATKQEGLESVLK | TGAACCAGCTGTCGAAGAAGGAGGCGTCGCGG |
| | ILVNQLSVDDVNVLTCATGTLSN | CGGGCCCTGATGGGCTCGCCCCAGCTGGTGGCC |
| | LTCNNSKNKTLVTQNSGVEALIH | GCTGTCGTGCGTACCATGCAGAATACCAGCGA |
| | AILRAGDKDDITEPAVCALRHLTS | CCTGGACACAGCCCGCTGCACCACCAGCATCCT |
| | RHPEAEMAQNSVRLNY | GCACAACCTCTCCCACCACCGGGAGGGGCTGC |
| | GIPAIVKLLNQPNQWPLVKATIGL | TCGCCATCTTCAAGTCGGGTGGCATCCCTGCTC |
| | IRNLALCPANHAPLQEAAVIPRLV | TGGTCCGCATGCTCAGCTCCCCTGTGGAGTCGG |
| | QLLVKAHQDAQRHVAAGTQQPY | TCCTGTTCTATGCCATCACCACGCTGCACAACC |
| | TDGVRMEEIVEGCTGALHILARD | TGCTCCTGTACCAGGAGGGCGCCAAGATGGCC |
| | PMNRMEIFRLNTIPLFVQLLYSSV | GTGCGCCTGGCCGACGGGCTGCAAAAGATGGT |
| | ENIQRVAAGVLCELAQDKEAADA | GCCCCTGCTCAACAAGAACAACCCCAAGTTCCT |
| | IDAEGASAPLMELLHSRNEGTAT | GGCCATCACCACCGACTGCCTGCAGCTCCTGGC |
| | YAAAVLFRISEDKNPDYRKRVSV | CTACGGCAACCAGGAGAGCAAGCTGATCATCC |
| | ELTNSLFKHDPAAWEAAQSMIPIN | TGGCCAATGGTGGGCCCCAGGCCCTCGTGCAG |
| | EPYGDDMDATYRPMYSSDVPLDP | ATCATGCGTAACTACAGTTATGAAAAGCTGCTC |
| | LEMHMDMDGDYPIDTYSDGLRPP | TGGACCACCAGTCGTGTGCTCAAGGTGCTATCC |
| | YPTADHMLA (SEQ ID NO: 226) | GTGTGTCCCAGCAATAAGCCTGCCATTGTGGAG |
| | | GCTGGTGGGATGCAGGCCCTGGGCAAGCACCT |
| | | GACCAGCAACAGCCCCCGCCTGGTGCAGAACT |
| | | GCCTGTGGACCCTGCGCAACCTCTCAGATGTGG |
| | | CCACCAAGCAGGAGGGCCTGGAGAGTGTGCTG |
| | | AAGATTCTGGTGAATCAGCTGAGTGTGGATGA |
| | | CGTCAACGTCCTCACCTGTGCCACGGGCACACT |
| | | CTCCAACCTGACATGCAACAACAGCAAGAACA |
| | | AGACGCTGGTGACACAGAACAGCGGTGTGGAG |
| | | GCTCTCATCCATGCCATCCTGCGTGCTGGTGAC |
| | | AAGGACGACATCACGGAGCCTGCCGTCTGCGC |
| | | TCTGCGCCACCTCACTAGCCGCCACCCTGAGGC |
| | | CGAGATGGCCCAGAACTCTGTGCGTCTCAACTA |
| | | TGGCATCCCAGCCATCGTGAAGCTGCTCAACCA |
| | | GCCCAACCAGTGGCCACTGGTCAAGGCAACCA |
| | | TCGGCTTGATCAGGAATCTGGCCCTGTGCCCAG |
| | | CCAACCATGCCCCGCTGCAGGAGGCAGCGGTC |
| | | ATCCCCCGCCTCGTCCAACTGCTGGTGAAGGCC |
| | | CACCAGGATGCCCAGCGCCACGTAGCTGCAGG |
| | | CACACAGCAGCCCTACACGGATGGTGTGAGGA |
| | | TGGAGGAGATTGTGGAGGGCTGCACCGGAGCA |
| | | CTGCACATCCTCGCCCGGGACCCCATGAACCGC |
| | | ATGGAGATCTTCCGGCTCAACACCATTCCCCTG |
| | | TTTGTGCAGCTCCTGTACTCGTCGGTGGAGAAC |
| | | ATCCAGCGCGTGGCTGCCGGGGTGCTGTGTGA |
| | | GCTGGCCCAGGACAAGGAGGCGGCCGACGCCA |
| | | TTGATGCAGAGGGGGCCTCGGCCCCACTCATG |
| | | GAGTTGCTGCACTCCCGCAACGAGGGCACTGC |
| | | CACCTACGCTGCTGCCGTCCTGTTCCGCATCTC |
| | | CGAGGACAAGAACCCAGACTACCGGAAGCGCG |
| | | TGTCCGTGGAGCTCACCAACTCCCTCTTCAAGC |
| | | ATGACCCGGCTGCCTGGGAGGCTGCCCAGAGC |
| | | ATGATTCCCATCAATGAGCCCTATGGAGATGAC |
| | | ATGGATGCCACCTACCGCCCCATGTACTCCAGC |
| | | GATGTGCCCCTTGACCCGCTGGAGATGCACATG |
| | | GACATGGATGGAGACTACCCCATCGACACCTA |
| | | CAGCGACGGCCTCAGGCCCCCGTACCCCACTGC |
| | | AGACCACATGCTGGCCTAG (SEQ ID NO: 225) |
| Human | MSGGRFDFDDGGAYCGGWEGGK | ATGAGTGGGGGCCGCTTCGACTTTGATGATGGA |
| JPH2 N- | AHGHGLCTGPKGQGEYSGSWNF | GGGGCGTACTGCGGGGGCTGGGAGGGGGGAAA |
| terminal | GFEVAGVYTWPSGNTFEGYWSQ | GGCCCATGGGCATGGACTGTGCACAGGCCCCA |
| fragment | GKRHGLGIETKGRWLYKGEWTH | AGGGCCAGGGCGAATACTCTGGCTCCTGGAAC |
| | GFKGRYGIRQSSSSGAKYEGTWN | TTTGGCTTTGAGGTGGCAGGTGTCTACACCTGG |
| | NGLQDGYGTETYADGGTYQGQF | CCCAGCGGAAACACCTTTGAGGGATACTGGAG |
| | TNGMRHGYGVRQSVPYGMAVV | CCAGGGCAAACGGCATGGGCTGGGCATAGAGA |
| | VRSPLRTSLSSLRSEHSNGTVAPD | CCAAGGGGCGCTGGCTCTACAAGGGCGAGTGG |
| | SPASPASDGPALPSPAIPRGGFALS | ACACATGGCTTCAAGGGACGCTACGGAATCCG |
| | LLANAEAAARAPKGGGLFQRGA | GCAGAGCTCAAGCAGCGGTGCCAAGTATGAGG |
| | LLGKLRRAESRTSVGSQRSRVSFL | GCACCTGGAACAATGGCCTGCAAGACGGCTAT |
| | KSDLSSGASDAASTASLGEAAEG | GGCACCGAGACCTATGCTGATGGAGGGACGTA |
| | ADEAAPFEADIDATTTETYMGEW | CCAAGGCCAGTTCACCAACGGCATGCGCCATG |
| | KNDKRSGFGVSERSSGLRYEGEW | GCTACGGAGTACGCCAGAGCGTGCCCTACGGG |
| | LDNLRHGYGCTTLPDGHREEGKY | ATGGCCGTGGTGGTGCGCTCGCCGCTGCGCACG |
| | RHNVLVKDTKRRMLQLKSNKVR | TCGCTGTCGTCCCTGCGCAGCGAGCACAGCAAC |
| | QKVEHSVEGAQRAAAIARQKAEI | GGCACGGTGGCCCCGGACTCTCCCGCCTCGCCG |
| | AASRTSHAKAKAEAAEQAALAA | GCCTCCGACGGCCCCGCGCTGCCCTCGCCCGCC |
| | NQESNIARTLARELAPDFYQPGPE | ATCCCGCGTGGCGGCTTCGCGCTCAGCCTCCTG |
| | YQKRRLLQEILENSESLLEPPDRG | GCCAATGCCGAGGCGGCCGCGCGGGCGCCCAA |
| | AGAAGLPQPPRESPQLHERETPRP | GGGCGGCGGCCTCTTCCAGCGGGGCGCGCTGC |
| | EGGSPSPAGTPPQPKRPRPGVSKD | TGGGCAAGCTGCGGCGCGCAGAGTCGCGCACG |
| | GLLSPGAWNGEPSGEGSRSVTPSE | TCCGTGGGTAGCCAGCGCAGCCGTGTCAGCTTC |
| | GAGRRSPARPATERMAIEALQAP | CTTAAGAGCGACCTCAGCTCGGGCGCCAGCGA |
| | PAPSREPEVALYQGYHSYAVR | CGCCGCGTCCACCGCCAGCCTGGGAGAGGCCG |
| | (SEQ ID NO: 228) | CCGAGGGCGCCGACGAGGCCGCACCCTTCGAG |
| | | GCCGATATCGACGCCACCACCACCGAGACCTA |
| | | CATGGGCGAGTGGAAGAACGACAAACGCTCGG |
| | | GCTTCGGCGTGAGCGAACGCTCCAGTGGCCTCC |
| | | GCTACGAGGGCGAGTGGCTGGACAACCTGCGC |
| | | CACGGCTATGGCTGCACCACGCTGCCCGACGG |
| | | CCACCGCGAGGAGGGCAAGTACCGCCACAACG |
| | | TGCTGGTCAAGGACACCAAGCGCCGCATGCTG |
| | | CAGCTCAAGAGCAACAAGGTCCGCCAGAAAGT |
| | | GGAGCACAGTGTGGAGGGTGCCCAGCGCGCCG |
| | | CTGCTATCGCGCGCCAGAAGGCCGAGATTGCC |
| | | GCCTCCAGGACAAGCCACGCCAAGGCCAAAGC |
| | | TGAGGCAGCGGAACAGGCCGCCCTGGCTGCCA |
| | | ACCAGGAGTCCAACATTGCTCGCACTTTGGCCA |
| | | GGGAGCTGGCTCCGGACTTCTACCAGCCAGGTC |
| | | CGGAATATCAGAAGCGCCGGCTGCTGCAGGAG |
| | | ATCCTGGAGAACTCGGAGAGCCTGCTGGAGCC |
| | | CCCCGACCGGGGCGCCGGCGCAGCGGGCCTCC |
| | | CACAGCCGCCCCGCGAGAGCCCGCAGCTGCAC |
| | | GAGCGTGAGACCCCTCGGCCCGAGGGTGGCTC |
| | | CCCGTCACCGGCCGGGACGCCCCCGCAGCCCA |
| | | AGCGGCCCAGGCCCGGGGTGTCCAAGGACGGC |
| | | CTGCTGAGCCCAGGCGCCTGGAACGGCGAGCC |
| | | CAGCGGTGAGGGCAGCCGGTCAGTCACTCCGT |
| | | CCGAGGGCGCGGGCCGCCGCAGCCCCGCGCGT |
| | | CCAGCCACCGAGCGCATGGCCATCGAGGCTCT |
| | | GCAGGCACCGCCTGCGCCGTCGCGGGAGCCGG |
| | | AGGTGGCGCTTTACCAGGGCTACCACAGCTATG |
| | | CTGTGCGC (SEQ ID NO: 227) |
| Human PLN | MEKVQYLTRSAIRRASTIEMPQQ | ATGGAGAAAGTCCAATACCTCACTCGCTCAGCT |
| | ARQKLQNLFINFCLILICLLLICIIV | ATAAGAAGAGCCTCAACCATTGAAATGCCTCA |
| | MLL(SEQ ID NO: 230) | ACAAGCACGTCAAAAGCTACAGAATCTATTTAT |
| | | CAATTTCTGTCTCATCTTAATATGTCTCTTGCTG |
| | | ATCTGTATCATCGTGATGCTTCTCTGA |
| | | (SEQ ID NO: 229) |

### Promoters and Enhancers

In some embodiments, the expression cassette of the disclosure comprises a promoter. The term “promoter” as used herein refers to a DNA sequence that directs the binding of RNA polymerase and thereby promotes RNA synthesis. Promoters and corresponding protein or polypeptide expression may be ubiquitous, meaning strongly active in a wide range of cells, tissues and species or cell-type specific, tissue-specific, or species specific. Examples of ubiquitous promoters include the CAG promoter and CMB promoter (Yue et al. BioTechniques 33:672-678 (2002)). Promoters may be “constitutive,” meaning continually active, or “inducible,” meaning the promoter can be activated or deactivated by the presence or absence of biotic or abiotic factors. Also included in the nucleic acid constructs or vectors of the invention are enhancer sequences that may or may not be contiguous with the promoter sequence. Enhancer sequences influence promoter-dependent gene expression and may be located in the 5′ or 3′ regions of the native gene.

Various promoters may be used. The promoter may be cell-type specific.

Constitutive promoters are used in expression cassettes and can be, for example, the cytomegalovirus enhancer fused to the chicken 3-actin promoter (CAG), simian virus 40 (SV40) promoter, and the herpes simplex virus thymidine kinase (HSV-TK) promoter (Damdindorj et al. PLoS One. 9:e106472 (2014)). Other cell-type specific promoters may also be used. Cardiac cell specific promoters can be, for example, the MLC2v promoter (Phillips et al. Hypertension 39:651-5 (2002)) and the cardiac Troponin-T (cTnT) promoter (Konkalmatt et al. Circ Cardiovasc Imaging. 6:478-486 (2013)). The transgene polynucleotide sequence in an expression cassette can be, for example, an open reading frame encoding a protein. The ITRs in an expression cassette serve as markers used for viral packaging of the expression cassette (Clark et al. Hum Gene Ther. 6:1329-41 (1995)).

Advantageously, the promoter, optionally in conjunction with an enhancer, enables expression of the polynucleotide encoding a polypeptide (e.g., a DWORF polypeptide), or functional variant thereof, in a target cell.

In some embodiments, the expression cassette comprises a single promoter. In some embodiments, the expression cassette comprises at least one promoter. In some embodiments, the expression cassette comprises two promoters. In some embodiments, the expression cassette comprises a ubiquitous promoter. In some embodiments, the expression cassette comprises an inducible promoter. In some embodiments, the expression cassette comprises a cell-type specific promoter. In some embodiments, the promoter specifically promotes expression of the polynucleotide encoding a polypeptide, or functional variant thereof, in a cardiac cell (e.g., a cardiomyocyte). In some embodiments, the promoter specifically promotes expression of the polynucleotide encoding the DWORF polypeptide, or functional variant thereof, in a cardiac cell. In some embodiments, the promoter specifically promotes expression of the polynucleotide encoding the DWORF polypeptide, or functional variant thereof, in a cardiomyocyte. Illustrative promoter and enhancer sequences are provided in Table 3.

In some embodiments, the promoter is a chicken cardiac troponin-T (cTnT or ccTnT) promoter. In some embodiments, the chicken cTnT promoter comprises a polynucleotide sequence that shares at least 90%, 95%, 96%, 97%, 98%, 99%, or 100% identity to SEQ ID NO: 11. In some embodiments, the chicken cTnT promoter comprises SEQ ID NO: 11.

In some embodiments, the promoter is a human cTnT promoter. In some embodiments, the promoter is a short human cTnT promoter. In some embodiments, the short human cTnT promoter comprises a polynucleotide sequence that shares at least 90%, 95%, 96%, 97%, 98%, 99%, or 100% identity to SEQ ID NO: 12. In some embodiments, the short human cTnT promoter comprises SEQ ID NO: 12. In some embodiments, the promoter is a long human cTnT promoter. In some embodiments, the long human cTnT promoter comprises a polynucleotide sequence that shares at least 90%, 95%, 96%, 97%, 98%, 99%, or 100% identity to SEQ ID NO: 13. In some embodiments, the long human cTnT promoter comprises SEQ ID NO: 13.

The expression cassette can include one or more enhancers. The term “enhancer” as used herein refers to a DNA sequence that directs the binding of transcriptional regulatory proteins (e.g., transcriptional machinery) and RNA polymerase, and thereby promotes RNA synthesis. The enhancer can be operably linked to a promoter and modulate the expression of a transgene operably linked to a promoter. The presence of an enhancer can modulate transgene expression by, for example, increasing expression or decreasing expression. An enhancer can modulate transgene expression by, for example, increasing expression levels in a desired cell type, for example, a cardiac cell. An enhancer can modulate transgene expression by, for example, decreasing expression levels in an “off-target” cell type, or a cell type in which expression is not desired.

In some embodiments, the expression cassette comprises a single enhancer. In some embodiments, the expression cassette comprises at least one enhancer. In some embodiments, the expression cassette comprises two enhancers. In some embodiments, the expression cassette comprises three enhancers. In some embodiments, the expression cassette comprises four enhancers. In some embodiments, the expression cassette comprises an enhancer that is operably linked to a promoter. For example, a ACTC1 cardiac enhancer can be linked to a human cTnT promoter. In some embodiments, the expression cassette comprises an enhancer that is operably linked to another enhancer. For example, a ACTC1 cardiac enhancer can be operably linked to an αMHC enhancer. In some embodiments, the expression cassette comprises an enhancer that is operably linked to a promoter and operably linked to another enhancer.

In some embodiments, the enhancer comprises an ACTC1 cardiac enhancer (ACTC1e). In some embodiments, the ACTC1 cardiac enhancer shares at least 90%, 95%, 96%, 97%, 98%, 99%, or 100% identity to SEQ ID NO: 78. In some embodiments, the ACTC1 cardiac enhancer comprises SEQ ID NO: 78. In some embodiments, the enhancer comprises an αMHC enhancer (αMHCe). In some embodiments, the αMHC enhancer shares at least 90%, 95%, 96%, 97%, 98%, 99%, or 100% identity to SEQ ID NO: 79. In some embodiments, the αMHC enhancer comprises SEQ ID NO: 79.

TABLE 3
Illustrative Promoter and Enhancer Sequences

| Promoter/ | |
|---|---|
| Enhancer | |
| Name | Sequences |
| Chicken cTnT | GGGATAAAAGCAGTCTGGGCTTTCACATGA |
| | CAGCATCTGGGGCTGCGGCAGAGGGTCGGG |
| | TCCGAAGCGCTGCCTTATCAGCGTCCCCAG |
| | CCCTGGGAGGTGACAGCTGGCTGGCTTGTG |
| | TCAGCCCCTCGGGCACTCACGTATCTCCGT |
| | CCGACGGGTTTAAAATAGCAAAACTCTGAG |
| | GCCACACAATAGCTTGGGCTTATATGGGCT |
| | CCTGTGGGGGAAGGGGGAGCACGGAGGGGG |
| | CCGGGGCCGCTGCTGCCAAAATAGCAGCTC |
| | ACAAGTGTTGCATTCCTCTCTGGGCGCCGG |
| | GCACATTCCTGCTGGCTCTGCCCGCCCCGG |
| | GGTGGGCGCCGGGGGGACCTTAAAGCCTCT |
| | GCCCCCCAAGGAGCCCTTCCCAGACAGCCG |
| | CCGGCACCCACCGCTCCGTGGGA |
| | (SEQ ID NO: 11) |
| Short Human | GTCATGGAGAAGACCCACCTTGCAGATGTC |
| cTnT | CTCACTGGGGCTGGCAGAGCCGGCAACCTG |
| | CCTAAGGCTGCTCAGTCCATTAGGAGCCAG |
| | TAGCCTGGAAGATGTCTTTACCCCCAGCAT |
| | CAGTTCAAGTGGAGCAGCACATAACTCTTG |
| | CCCTCTGCCTTCCAAGATTCTGGTGCTGAG |
| | ACTTATGGAGTGTCTTGGAGGTTGCCTTCT |
| | GCCCCCCAACCCTGCTCCCAGCTGGCCCTC |
| | CCAGGCCTGGGTTGCTGGCCTCTGCTTTAT |
| | CAGGATTCTCAAGAGGGACAGCTGGTTTAT |
| | GTTGCATGACTGTTCCCTGCATATCTGCTC |
| | TGGTTTTAAATAGCTTATCTGAGCAGCTGG |
| | AGGACCACATGGGCTTATATGGCGTGGGGT |
| | ACATGTTCCTGTAGCCTTGTCCCTGGCACC |
| | TGCCAAAATAGCAGCCAACACCCCCCACCC |
| | CCACCGCCATCCCCCTGCCCCACCCGTCCC |
| | CTGTCGCACATTCCTCCCTCCGCAGGGCTG |
| | GCTCACCAGGCCCCAGCCCACATGCCTGCT |
| | TAAAGCCCTCTCCATCCTCTGCCTCACCCA |
| | GTCCCCGCTGAGACTGAGCAGACGCCTCCA |
| | (SEQ ID NO: 12) |
| Long Human | AGAGGACCCTTTCAAGGACATTAGTGGTGG |
| cTnT | AGGCAGCATAGTAGCTCCCAAGGCAGAGGG |
| | ATTGAGAGAAGAGTTTGAGGACTGGGAAGG |
| | CGGGACACATGATTGGGTGATGGGAGAAGG |
| | GGGCAGAGAATAGCGAGATTGCTTTCTTTG |
| | CCCACGGAGAAACAGAGGAGTGTGGATCAT |
| | GAATGGGCAAGATCTTTAAGTGCCAGGGGG |
| | GGTCATGGAGGAGGGGAGGGCCTGCTCCAG |
| | AGGAGGACCATTCCTGCTTCAGAGCCAAGC |
| | AGGACCTAGGCTGTGAAGATTCGGAGAAAG |
| | AGATGGAGGGGAGAGTCAGCTCAGCTGCTT |
| | ACTGGCTTGCTTTCCTCCTGTCTCTTTCAT |
| | TTTCATAATCTACCAAACCCTGCAATGGGC |
| | CAGCCTTGAACATACAAGTGCATGTGCATG |
| | GTCAGACACAGGCAAGCAAGCAAGACCCCT |
| | AGGCCTGACCTATGCATCTGCAATCTAGTA |
| | GGTTTAGCAGATCATAGCCCCGCACTGCTT |
| | GATTTTAAAGCCGTTAGGGGATGACCTTTG |
| | ACAGTCCGCATCACCCCTCTCACACAACGA |
| | GCGCCTGTTCAAGGTTCTTGACTGGAAGTT |
| | CTACCTTGTATCTGGCCTCCTGTAGCAGTT |
| | TCAGTCCATTCCCTGTGAGGAGGGTGTGCC |
| | ACATGGCTTTGGGGGTCATGGAGAAGACCC |
| | ACCTTGCAGATGTCCTCACTGGGGCTGGCA |
| | GAGCCGGCAACCTGCCTAAGGCTGCTCAGT |
| | CCATTAGGAGCCAGTAGCCTGGAAGATGTC |
| | TTTACCCCCAGCATCAGTTCAAGTGGAGCA |
| | GCACATAACTCTTGCCCTCTGCCTTCCAAG |
| | ATTCTGGTGCTGAGACTTATGGAGTGTCTT |
| | GGAGGTTGCCTTCTGCCCCCCAACCCTGCT |
| | CCCAGCTGGCCCTCCCAGGCCTGGGTTGCT |
| | GGCCTCTGCTTTATCAGGATTCTCAAGAGG |
| | GACAGCTGGTTTATGTTGCATGACTGTTCC |
| | CTGCATATCTGCTCTGGTTTTAAATAGCTT |
| | ATCTGAGCAGCTGGAGGACCACATGGGCTT |
| | ATATGGCGTGGGGTACATGTTCCTGTAGCC |
| | TTGTCCCTGGCACCTGCCAAAATAGCAGCC |
| | AACACCCCCCACCCCCACCGCCATCCCCCT |
| | GCCCCACCCGTCCCCTGTCGCACATTCCTC |
| | CCTCCGCAGGGCTGGCTCACCAGGCCCCAG |
| | CCCACATGCCTGCTTAAAGCCCTCTCCATC |
| | CTCTGCCTCACCCAGTCCCCGCTGAGACTG |
| | AGCAGACGCCTCCAGGATCTGTCGGCAGCT |
| | GCTGTTCTGAGGTAAGGCTCGGGCAGGGCT |
| | CTGGGGAAGAGGAGAGCAGAGAATGGACGG |
| | GGAGATGTGAGGGTCTTGGGCCCTGGCATA |
| | TTTACCCAGAGTCTGCCTGTGTCCGCAGAA |
| | GTCCATGGCCCCTCCTGGTGGAGGCCACAC |
| | TTCAGAGGACAGGTTGCCAGGTCTGGGCTC |
| | CAAGATTGGTACAATAGAGCAGAGAGA |
| | (SEQ ID NO: 13) |
| ACTC1 cardiac | AACTGGCCTGCCCGAGACCAAACGTGCGGA |
| enhancer | ACGTAGTTAAGTGTTAGAGGTAGGATTTGA |
| (ACTC1e) | AGCCTGTCGATCATTCTGATTCTCCTTTTC |
| | TCTACGTCTGCTTCCTGTCAATGGGCATCC |
| | TCACTGTCAAATGCAGATGGTACAGCAGGG |
| | CTTGGTCTCAGCCAGGCAGGCCTCTCCCCA |
| | GTCTCCATGGCTCAGCTGTCCAGCAGTTTC |
| | ATCCCTAGACCATCCCAAACATGGTTGAGA |
| | AGCTCTGAGGGGAGGACCCAGCACTGCCCG |
| | GCCCCTGAAGATAATCAGCAGTCCTGCTCA |
| | GCATATCAATCCAAGCCCACTCTAGACAGA |
| | GATGCCGGTGCCCAGTTTTCTATTTTTAAC |
| | TGGTGTGAACTGAAGGAAAAGCACAGCATT |
| | AGAAGTCCAAGCA |
| | (SEQ ID NO: 78) |
| αMHC cardiac | CCTTCAGATTAAAAATAACTAAGGTAAGGG |
| enhancer | CCATGTGGGTAGGGGAGGTGGTGTGAGACG |
| (αMHCe) | GTCCTGTCTCTCCTCTATCTGCCCATCGGC |
| | CCTTTGGGGAGGAGGAATGTGCCCAAGGAC |
| | TAAAAAAAGGCCCTGGAGCCAGAGGGGCGA |
| | GGGCAGCAGACCTTTCATGGGCAAACCTCA |
| | GGGCTGCTGTC |
| | (SEQ ID NO: 79) |

### Introns

The expression cassette can include an intron sequence, for example, a synthetic or chimeric intron sequence. The intron sequence can be used to adjust the length (i.e., size) of the expression cassette for improving recombinant AAV packaging. The intron sequence can be used to improve the efficiency of transgene expression (i.e., mRNA production or transcription) in a host cell containing the expression cassette. In some embodiments, the expression cassette comprises an intron. In some embodiments, the intron comprises the CMV intron (CMVint). In some embodiments, the CMV intron shares at least 90%, 95%, 96%, 97%, 98%, 99%, or 100% identity to SEQ ID NO: 80. In some embodiments, the CMV intron comprises SEQ ID NO: 80. In some embodiments, the intron comprises a chimeric intron. In some embodiments, the chimeric intron shares at least 90%, 95%, 96%, 97%, 98%, 99%, or 100% identity to SEQ ID NO: 81. In some embodiments, the chimeric intron comprises SEQ ID NO: 81.

TABLE 9
Illustrative Intron Sequences

| Intron | Intron Sequence |
|---|---|
| CMV intron | GTAAGTACCGCCTATAGACT |
| | CTATAGGCACACCCCTTTGG |
| | CTCTTATGCATGCTGACAGA |
| | CTAACAGACTGTTCCTTTCC |
| | TGGGTCTTTTCTGCAG |
| | (SEQ ID NO: 80) |
| Chimeric Intron | GTAAGTATCAAGGTTACAAG |
| (Chimint) | ACAGGTTTAAGGAGACCAAT |
| | AGAAACTGGGCTTGTCGAGA |
| | CAGAGAAGACTCTTGCGTTT |
| | CTGATAGGCACCTATTGGTC |
| | TTACTGACATCCACTTTGCC |
| | TTTCTCTCCACAG |
| | (SEQ ID NO: 81) |

### WPRE Sequences and Other Post-Transcriptional Elements

In some embodiments, the expression cassette comprises a posttranscriptional regulatory element.

In some embodiments, the expression cassette comprises a woodchuck hepatitis virus post-transcriptional element (WPRE). The WPRE sequence can be inserted, for example, proximal to on the 3′ end of a transgene in a viral vector to, for example, optimize gene expression in a viral vector (Lee et al. Exp Physiol. 90:33-37 (2005)). In some embodiments, the WPRE comprises a polynucleotide sequence that shares at least 90%, 95%, 96%, 97%, 98%, 99%, or 100% identity to SEQ ID NO: 26. In some embodiments, the WPRE comprises SEQ ID NO: 26.

TABLE 4
Illustrative WPRE Sequence
WPRE Sequence

TCAACCTCTGGATTACAAAATTTGTGAAAGATTGACTGGTATTCT
TAACTATGTTGCTCCTTTTACGCTATGTGGATACGCTGCTTTAAT
GCCTTTGTATCATGCTATTGCTTCCCGTATGGCTTTCATTTTCTC
CTCCTTGTATAAATCCTGGTTGCTGTCTCTTTATGAGGAGTTGTG
GCCCGTTGTCAGGCAACGTGGCGTGGTGTGCACTGTGTTTGCTGA
CGCAACCCCCACTGGTTGGGGCATTGCCACCACCTGTCAGCTCCT
TTCCGGGACTTTCGCTTTCCCCCTCCCTATTGCCACGGCGGAACT
CATCGCCGCCTGCCTTGCCCGCTGCTGGACAGGGGCTCGGCTGTT
GGGCACTGACAATTCCGTGGTGTTGTCGGGGAAATCATCGTCCTT
TCCTTGGCTGCTCGCCTGTGTTGCCACCTGGATTCTGCGCGGGAC
GTCCTTCTGCTACGTCCCTTCGGCCCTCAATCCAGCGGACCTTCC
TTCCCGCGGCCTGCTGCCGGCTCTGCGGCCTCTTCCGCGTCTTCG
(SEQ ID NO: 26)

### Poly Adenylation Sequences

In some embodiments, the expression cassette comprises a poly(A) signal sequence. In some embodiments, the poly(A) signal is a BGH poly(A) sequence. In some embodiments, the BGH poly(A) signal sequence comprises the polynucleotide sequence that shares at least 90%, 95%, 96%, 97%, 98%, 99%, or 100% identity to SEQ ID NO: 27. In some embodiments, the poly(A) signal is an SV40 poly(A) signal. In some embodiments, the SV40 poly(A) signal sequence comprises the polynucleotide sequence that shares at least 90%, 95%, 96%, 97%, 98%, 99%, or 100% identity to SEQ ID NO: 28.

TABLE 5
Illustrative Poly(A) Sequences

| Poly(A) | |
|---|---|
| Sequence | Sequence |
| BGH | GCCTCGACTGTGCCTTCTAG |
| | TTGCCAGCCATCTGTTGTTT |
| | GCCCCTCCCCCGTGCCTTCC |
| | TTGACCCTGGAAGGTGCCAC |
| | TCCCACTGTCCTTTCCTAAT |
| | AAAATGAGGAAATTGCATCG |
| | CATTGTCTGAGTAGGTGTCA |
| | TTCTATTCTGGGGGGTGGGG |
| | TGGGGCAGGACAGCAAGGGG |
| | GAGGATTGGGAAGACAATAG |
| | CAGGCATGCTGGGGA |
| | (SEQ ID NO: 27) |
| SV40 | GATCCAGACATGATAAGATA |
| | CATTGATGAGTTTGGACAAA |
| | CCACAACTAGAATGCAGTGA |
| | AAAAAATGCTTTATTTGTGA |
| | AATTTGTGATGCTATTGCTT |
| | TATTTGTAACCATTATAAGC |
| | TGCAATAAACAAGT |
| | (SEQ ID NO: 28) |

### Inverted Terminal Repeat Sequences

In some embodiments, the expression cassette is flanked by AAV inverted terminal repeats (ITRs). In some embodiments, the ITRs comprise the polynucleotide sequence that shares at least 90%, 95%, 96%, 97%, 98%, 99%, or 100% identity to SEQ ID NO: 14 and/or SEQ ID NO: 15.

TABLE 6
Illustrative ITR Sequences
ITR Sequences

CTGCGCGCTCGCTCGCTCAC
TGAGGCCGCCCGGGCAAAGC
CCGGGCGTCGGGCGACCTTT
GGTCGCCCGGCCTCAGTGAG
CGAGCGAGCGCGCAGAGAGG
GAGTGGCCAACTCCATCACT
AGGGGTTCCT
(SEQ ID NO: 14)
AGGAACCCCTAGTGATGGAG
TTGGCCACTCCCTCTCTGCG
CGCTCGCTCGCTCACTGAGG
CCGGGCGACCAAAGGTCGCC
CGACGCCCGGGCTTTGCCCG
GGCGGCCTCAGTGAGCGAGC
GAGCGCGCAG
(SEQ ID NO: 15)

### Illustrative Expression Cassettes

The disclosure provides expression cassettes comprising a polynucleotide comprising a 5′ to 3′ arrangement (sometimes referred to as an orientation) of elements. In some embodiments, the elements comprise one or more promoters; optionally one or more enhancers; optionally one or more introns; one or more transgenes; optionally one or more WPRE sequences; and optionally one or more polyadenylation sequences (p(A)). Illustrative order of the elements in the polynucleotide are shown in FIG. 1, FIG. 7A, FIG. 7B, FIG. 7C and Table 1. Illustrative orientations of the elements on the polynucleotide are also shown in FIG. 1, FIG. 7A, FIG. 7B, FIG. 7C and Table 1. In some embodiments, the 5′ to 3′ arrangement of elements is selected from:
5′-promoter-transgene-WPRE-p(A)-3′;
5′-promoter-intron-transgene-WPRE-p(A)-3′;
5′-promoter-transgene-WPRE-p(A)-promoter-transgene-WPRE-p(A);
5′-enhancer-promoter-transgene-WPRE-p(A)-3′;
5′-enhancer-promoter-intron-transgene-WPRE-p(A)-3′;
5′-enhancer-enhancer-promoter-transgene-WPRE-p(A)-3′;
5′-enhancer-enhancer-promoter-intron-transgene-WPRE-p(A)-3′;
5′-enhancer-promoter-intron-transgene-WPRE-p(A)-p(A)-transgene-intron-promoter-enhancer-3′;
5′-enhancer-promoter-intron-transgene-WPRE-p(A)-enhancer-promoter-intron-transgene-p(A)-3′;
5′-p(A)-WPRE-transgene-intron-promoter-enhancer-enhancer-promoter-intron-transgene-p(A)-3′;
5′-promoter-intron-transgene-WPRE-p(A)-p(A)-transgene-intron-promoter-3′;
5′-promoter-intron-transgene-WPRE-p(A)-promoter-intron-transgene-p(A)-3′; and
5′-p(A)-WPRE-transgene-intron-promoter-promoter-intron-transgene-p(A)-3′.

In some embodiments, the expression cassettes described herein achieve an increased expression level of the transgene compared to a second expression cassette comprising a polynucleotide having an arrangement of elements from 5′ to 3′ comprising: 5′-promoter-transgene-WPRE-p(A)-3′. In some embodiments, the expression level is increased between about 1.5-fold and about 150-fold compared the second expression cassette.

In some embodiments, the expression cassettes provided herein comprise the following elements (where the elements can be those described herein, e.g., the sequences of which are provided herein):
5′-promoter-transgene-WPRE-p(A)-3′;
5′-promoter-intron-transgene-WPRE-p(A)-3′;
5′-promoter-transgene-WPRE-p(A)-promoter-transgene-WPRE-p(A);
5′-enhancer-promoter-transgene-WPRE-p(A)-3′;
5′-enhancer-promoter-intron-transgene-WPRE-p(A)-3′;
5′-enhancer-enhancer-promoter-transgene-WPRE-p(A)-3′;
5′-enhancer-enhancer-promoter-intron-transgene-WPRE-p(A)-3′;
5′-enhancer-promoter-intron-transgene-WPRE-p(A)-p(A)-transgene-intron-promoter-enhancer-3′;
5′-enhancer-promoter-intron-transgene-WPRE-p(A)-enhancer-promoter-intron-transgene-p(A)-3′;
5′-p(A)-WPRE-transgene-intron-promoter-enhancer-enhancer-promoter-intron-transgene-p(A)-3′;
5′-promoter-intron-transgene-WPRE-p(A)-p(A)-transgene-intron-promoter-3′;
5′-promoter-intron-transgene-WPRE-p(A)-promoter-intron-transgene-p(A)-3′; or
5′-p(A)-WPRE-transgene-intron-promoter-promoter-intron-transgene-p(A)-3′.

In the expression cassettes described herein (such as those listed above), the orientation of the promoter, enhancer, transgene and poly(A) elements can be forward or reverse (e.g., in cases where there are more than one promoters, one promoter, optionally enhancer, and operably linked transgene can be oriented in a forward direction, and another promoter, optionally enhancer, and operably linked transgene can be oriented in a reverse direction).

In some embodiments, the expression cassettes provided herein comprise the following elements:
5′-cardiac-specific promoter-transgene-WPRE-p(A)-3′;
5′-cardiac-specific promoter-intron (e.g., chimeric intron)-transgene-WPRE-p(A)-3′;
5′-cardiac-specific promoter-transgene-WPRE-p(A)-promoter-transgene-WPRE-p(A), where both promoters and transgene sequences are in the same, forward orientation;
5′-cardiac-specific promoter-transgene-WPRE-p(A) (e.g., bGHpA)-3′;
5′-cardiac-specific promoter-intron (e.g., CMV intron)-transgene-WPRE-p(A) (e.g., bGHpA)-3′;
5′-enhancer (e.g., ACTC1e)-cardiac-specific promoter-transgene-WPRE-p(A) (e.g., bGHpA)-3′;
5′-enhancer (e.g., αMHCe)-cardiac-specific promoter-transgene-WPRE-p(A) (e.g., bGHpA)-3′;
5′-enhancer (e.g., ACTC1e)-cardiac-specific promoter-intron (e.g., CMV intron)-transgene-WPRE-p(A) (e.g., bGHpA)-3′;
5′-enhancer (e.g., αMHCe)-cardiac-specific promoter-intron (e.g., CMV intron)-transgene-WPRE-p(A) (e.g., bGHpA)-3′;
5′-enhancer (e.g., ACTC1e)-enhancer (e.g., αMHCe)-cardiac-specific promoter-transgene-WPRE-p(A) (e.g., bGHpA)-3′;
5′-enhancer (e.g., αMHCe)-enhancer (e.g., ACTC1e)-cardiac-specific promoter-transgene-WPRE-p(A) (e.g., bGHpA)-3′;
5′-enhancer (e.g., ACTC1e)-enhancer (e.g., αMHCe)-cardiac-specific promoter-intron (e.g., CMV intron)-transgene-WPRE-p(A) (e.g., bGHpA)-3′;
5′-enhancer (e.g., αMHCe)-enhancer (e.g., ACTC1e)-cardiac-specific promoter-intron (e.g., CMV intron)-transgene-WPRE-p(A) (e.g., bGHpA)-3′;
5′-cardiac-specific promoter-transgene with a codon-optimized polynucleotide sequence-WPRE-p(A) (e.g., bGHpA)-3′;
5′-enhancer (e.g., αMHCe)-cardiac-specific promoter-intron (e.g., CMV intron)-transgene-WPRE-p(A) (e.g., bGHpA)-p(A) (e.g., SV40 pA)-transgene (e.g., with a codon-optimized polynucleotide sequence)-intron (e.g., chimeric intron)-cardiac-specific promoter-enhancer (e.g., ACTC1e)-3′, optionally wherein the first in order transgene and the promoter/enhancer sequences operably linked thereto are in a forward orientation, and the second in order transgene and the promoter/enhancer sequences operably linked thereto are in a reverse orientation;
5′-enhancer (e.g., αMHCe)-cardiac-specific promoter-intron (e.g., CMV intron)-transgene-WPRE-p(A) (e.g., bGHpA)-enhancer (e.g., ACTC1e)-cardiac-specific promoter-intron (e.g., chimeric intron)-transgene (e.g., with a codon-optimized polynucleotide sequence)-p(A) (e.g., SV40 pA)-3′, optionally wherein both the first and the second in order transgenes and the promoter/enhancer sequences operably linked thereto are in a forward orientation;
5′-p(A) (e.g., bGHpA)-WPRE-transgene-intron (e.g., CMV intron)-cardiac-specific promoter-enhancer (e.g., αMHCe)-enhancer (e.g., ACTC1e)-cardiac-specific promoter-intron (e.g., chimeric intron)-transgene (e.g., with a codon-optimized polynucleotide sequence)-p(A) (e.g., SV40 pA)-3′, optionally wherein the first in order transgene and the promoter/enhancer sequences operably linked thereto are in a reverse orientation, and the second in order transgene and the promoter/enhancer sequences operably linked thereto are in a forward orientation;
5′-cardiac-specific promoter-intron (e.g., CMV intron)-transgene-WPRE-p(A) (e.g., bGHpA)-p(A) (e.g., SV40 pA)-transgene (e.g., with a codon-optimized polynucleotide sequence)-intron (e.g., chimeric intron)-cardiac-specific promoter-3′, optionally wherein the first in order transgene and the promoter/enhancer sequences operably linked thereto are in a forward orientation, and the second in order transgene and the promoter/enhancer sequences operably linked thereto are in a reverse orientation;
5′-cardiac-specific promoter-intron (e.g., CMV intron)-transgene-WPRE-p(A) (e.g., bGHpA)-cardiac-specific promoter-intron (e.g., chimeric intron)-transgene (e.g., with a codon-optimized polynucleotide sequence)-p(A) (e.g., SV40 pA)-3′, optionally wherein both the first and the second in order transgenes and the promoter/enhancer sequences operably linked thereto are in a forward orientation; or
5′-p(A) (e.g., bGHpA)-WPRE-transgene-intron (e.g., CMV intron)-cardiac-specific promoter-cardiac-specific promoter-intron (e.g., chimeric intron)-transgene (e.g., with a codon-optimized polynucleotide sequence)-p(A) (e.g., SV40 pA)-3′, optionally wherein the first in order transgene and the promoter/enhancer sequences operably linked thereto are in a reverse orientation, and the second in order transgene and the promoter/enhancer sequences operably linked thereto are in a forward orientation.

In the expression cassettes described herein (such as those listed above), the cardiac-specific promoter can be a short human cTnT promoter (such as hcTnTp) or chicken cTnT promoter (such as ccTnTp). The more specific examples of the expression cassettes described above can be found in, e.g., FIG. 1, FIG. 7A, FIG. 7B, FIG. 7C and Table 1.

In some embodiments, the expression cassettes described herein enable an increased expression level of the transgene compared to a second expression cassette comprising a polynucleotide having an arrangement of elements from 5′ to 3′ comprising: 5′-promoter-transgene-WPRE-p(A)-3′. In some embodiments, the expression level is increased between about 1.5-fold and about 150-fold compared the second expression cassette.

In some embodiments, one or more (e.g., one, two, three or four) elements of the expression cassettes described herein can be omitted.

In some embodiments, one or more (e.g., one, two, three or four) elements of the expression cassettes described herein can be replaced by other elements, such as functionally equivalent elements.

In some embodiments of the expression cassettes provided herein, the WPRE element is replaced by any other post-transcriptional regulatory element known in the art. In some embodiments, the expression cassettes provided herein comprise any post-transcriptional regulatory element known in the art. In some embodiments, the expression cassettes provided herein do not comprise a post-transcriptional regulatory element (e.g., do not comprise the WPRE element). In some embodiments, the expression cassettes provided herein comprise WPRE.

In some embodiments of the expression cassettes provided herein, the bGHpA and/or SV40 pA poly(A) element is replaced by any other poly(A) element known in the art. In some embodiments, the expression cassettes provided herein comprise any poly(A) element known in the art. In some embodiments, the expression cassettes provided herein do not comprise a poly(A) element. In some embodiments, the expression cassettes provided herein do not comprise bGHpA. In some embodiments, the expression cassettes provided herein do not comprise SV40 pA. In some embodiments, the expression cassettes provided herein do not comprise bGHpA or SV40 pA. In some embodiments, the expression cassettes provided herein comprise one or both of bGHpA and SV40 pA.

In some embodiments of the expression cassettes provided herein, the CMV intron and/or chimeric intron element is replaced by any other intron element known in the art. In some embodiments, the expression cassettes provided herein comprise any intron element known in the art. In some embodiments, the expression cassettes provided herein do not comprise an intron. In some embodiments, the expression cassettes provided herein do not comprise a CMV intron. In some embodiments, the expression cassettes provided herein do not comprise a chimeric intron (e.g., do not comprise Chim int). In some embodiments, the expression cassettes provided herein do not comprise CMV intron or Chim int. In some embodiments, the expression cassettes provided herein comprise one or both of CMV intron and Chim int.

It should be understood that the illustrative orientations of the expression cassette can include flanking inverted terminal repeat (ITR) sequences on the 5′ and 3′ ends of the expression cassette. It should be understood that the ITR sequences can be optional. In some embodiments, the expression cassettes described herein do not include the ITR sequences (e.g., non-AAV, such as DNA plasmid-based, expression cassettes).

Operably linked elements, such as those in the illustrative orientations above, can be on one or both strands of the polynucleotide.

In some embodiments, the expression cassette comprises one copy of a sequence encoding a polypeptide (i.e., one copy of a transgene). In some embodiments, the expression cassette comprises two copies of a sequence encoding a polypeptide (i.e., two copies of a transgene). In some embodiments, where the expression cassette comprises two copies of a sequence encoding a polypeptide, the two “copies” are not identical. While not being bound by any theory, using two sequences encoding a polypeptide that are not identical may prevent DNA recombination within the vector. In some embodiments, the expression cassette comprises one copy that has the original DNA sequence encoding a polypeptide and one copy that has a codon optimized DNA sequence encoding the polypeptide. In some embodiments, where the expression cassette comprises two copies of a sequence encoding a polypeptide, the two copies are identical.

In some embodiments, the expression cassette comprises one or more promoters described herein (with or without one or more enhancers described herein) driving one or more copies of a transgene (such as any transgene described herein). In some embodiments, the expression cassette does not comprise an enhancer (e.g., αMHCe and/or ACTC1e). In some embodiments, the expression cassette comprises one or more enhancers such as cardiac-specific enhancers (e.g., αMHCe and/or ACTC1e). In some embodiments, the expression cassette comprises αMHCe enhancer (and, optionally, does not comprise ACTC1e enhancer). In some embodiments, the expression cassette comprises ACTC1e enhancer (and, optionally, does not comprise αMHCe enhancer). In some embodiments, the expression cassette comprises at least two enhancers in the order of first αMHCe and then ACTC1e. In some embodiments, the expression cassette comprises at least two enhancers in the order of first ACTC1e and then αMHCe. In some embodiments, the expression cassette comprises an intron element, e.g., a CMV intron element and/or a chimeric intron (such as Chim int described herein). In some embodiments, the expression cassette comprises an intron element but does not comprise an enhancer. In some embodiments, the expression cassette comprises an intron element (e.g., CMV intron and/or a chimeric intron) and further comprises an enhancer (e.g., αMHCe and/or ACTC1e). In some embodiments, the expression cassette comprises a transgene with a codon-optimized polynucleotide sequence. In some embodiments, the expression cassette comprises a transgene with a codon-optimized polynucleotide sequence but does not comprise an enhancer. In some embodiments, the expression cassette comprises a transgene with a codon-optimized polynucleotide sequence and further comprises an enhancer (e.g., αMHCe and/or ACTC1e). In some embodiments, the expression cassette comprises one or more promoters described herein and one, two or more enhancers described herein (e.g., comprises an αMHCe and/or ACTC1e enhancer) driving the expression of one or more copies of a transgene (without or without CMV intron or chimeric intron elements). In some embodiments, the promoter is a cardiac-specific promoter, e.g., a human cTnT promoter (such as a short human promoter, hcTnTp) and/or a chicken cTnT promoter (such as ccTnTp). In some of the embodiments, the enhancer is a cardiac-specific enhancer, e.g., αMHCe and/or ACTC1e. In some embodiments, two or more cardiac-specific enhancers are used, where the two or more of the enhancers can be the same or different (e.g., both or all αMHCe, both or all ACTC1e, or at least one αMHCe and at least one ACTC1e). In some embodiments, two cardiac-specific enhancers are used, where the two enhancers can be the same or different (e.g., both αMHCe, both ACTC1e, or one αMHCe and one ACTC1e). In some embodiments, the transgene comprises a non-codon-optimized polynucleotide sequence encoding a gene product. In some embodiments, the order of the elements is as shown in any of the expression cassettes depicted in FIG. 1, FIG. 7A, FIG. 7B, FIG. 7C and Table 1. For example, (i) the order of the promoter and transgene elements can be as shown in FIG. 1, FIG. 7A, FIG. 7B, FIG. 7C or Table 1, (ii) the order of promoter, enhancer and transgene elements can be as shown in FIG. 1, FIG. 7A, FIG. 7B, FIG. 7C or Table 1, (iii) the order of promoter, transgene, WPRE and poly(A) elements can be as shown in FIG. 1, FIG. 7A, FIG. 7B, FIG. 7C or Table 1, optionally with or without enhancer elements being in the same order as shown in FIGi, FIG. 7A, FIG. 7B, FIG. 7C or Table 1, or (iv) the order of promoter, transgene, WPRE, poly(A), CMV intron (such as CMVint) elements can be as shown in FIG. 1, FIG. 7A, FIG. 7B, FIG. 7C or Table 1, optionally with or without enhancer elements being in the same order as shown in FIG. 1, FIG. 7A, FIG. 7B, FIG. 7C or Table 1. In some embodiments, the orientation of any of the elements is as shown in any of the expression cassettes depicted in FIG. 1, FIG. 7A, FIG. 7B, FIG. 7C and Table 1. The sequences of individual expression cassette elements discussed herein (such as promoters, enhancers, transgenes, WPRE, poly(A), CMV intron and chimeric intron) can be any of the sequences of such elements provided herein or any sequences with at least, e.g., 75%, 80%, 85%, 90%, 95%, 97%, 98%, 99% or 100% sequence identity thereto.

In some embodiments, the expression cassette comprises one promoter described herein (with or without one or more enhancers described herein) driving one copy of a transgene (such as any transgene described herein). In some embodiments, the expression cassette comprises one promoter described herein, without any enhancer (e.g., without any enhancer described herein, e.g., without αMHCe and/or without ACTC1e) driving one copy of a transgene, optionally, such an expression cassette comprises an intron element, e.g., a CMV intron element and/or a chimeric intron (such as Chim int described herein), and/or comprises a transgene with a codon-optimized polynucleotide sequence. In some embodiments, the expression cassette comprises one promoter described herein, without any enhancer (e.g., without any enhancer described herein, e.g., without αMHCe and/or without ACTC1e) driving one copy of a transgene, and further comprises a CMV intron and/or a chimeric intron (such as Chim int). In some embodiments, the expression cassette comprises one promoter described herein, without any enhancer (e.g., without any enhancer described herein, e.g., without αMHCe and/or without ACTC1e) driving one copy of a transgene, and further comprises a CMV intron. In some embodiments, the expression cassette comprises one promoter described herein, without any enhancer (e.g., without any enhancer described herein, e.g., without αMHCe and/or without ACTC1e) driving one copy of a transgene, wherein the transgene comprises a codon-optimized polynucleotide sequence. In some embodiments, the expression cassette comprises one promoter described herein and one, two or more enhancers described herein (e.g., comprises an αMHCe and/or ACTC1e enhancer) driving the expression of one copy of a transgene. In some embodiments, the expression cassette comprises one promoter described herein and one enhancer described herein (e.g., αMHCe or ACTC1e enhancer) driving the expression of one copy of a transgene. In some embodiments, the expression cassette comprises one promoter described herein and two enhancers described herein (e.g., both αMHCe, both ACTC1e, or one αMHCe and one ACTC1e) operably linked to one copy of a transgene. In some embodiments, the promoter is a cardiac-specific promoter, e.g., a human cTnT promoter (such as a short human promoter, hcTnTp) and/or a chicken cTnT promoter (such as ccTnTp). In some of the embodiments where one or more enhancers are used, the enhancer is a cardiac-specific enhancer, e.g., αMHCe and/or ACTC1e. In some embodiments, two or more cardiac-specific enhancers are used, where the two or more of the enhancers can be the same or different (e.g., both or all αMHCe, both or all ACTC1e, or at least one αMHCe and at least one ACTC1e). In some embodiments, two cardiac-specific enhancers are used, where the two enhancers can be the same or different (e.g., both αMHCe, both ACTC1e, or one αMHCe and one ACTC1e). In some embodiments, the expression cassette comprises at least two enhancers in the order of first αMHCe and then ACTC1e. In some embodiments, the expression cassette comprises at least two enhancers in the order of first ACTC1e and then αMHCe. In some embodiments, the transgene comprises a non-codon-optimized polynucleotide sequence encoding a gene product. In some embodiments, the transgene comprises a codon-optimized polynucleotide sequence encoding the gene product. In some embodiments, one or more intron elements are also used in addition to promoter and enhancer elements. In some embodiments, a CMV intron element is used. In some embodiments, a chimeric intron element (Chim int) is used. In some embodiments, both a CMV intron and a chimeric intron (Chim int) are used. In some embodiments where one promoter is used, the order of the elements is as shown in any of the expression cassettes depicted in FIG. 7B. For example, (i) the order of the promoter and transgene elements can be as shown in FIG. 7B, (ii) the order of promoter, enhancer and transgene elements can be as shown in FIG. 7B, (iii) the order of promoter, transgene, WPRE and poly(A) elements can be as shown in FIG. 7B, optionally with or without enhancer elements being in the same order as shown in FIG. 7B, or (iv) the order of promoter, transgene, WPRE, poly(A), CMV intron (such as CMVint) elements can be as shown in FIG. 7B, optionally with or without enhancer elements being in the same order as shown in FIG. 7B. In some embodiments where one promoter is used, the orientation of any of the elements is as shown in any of the expression cassettes depicted in FIG. 7B. In some embodiments, the orientation of the elements is forward orientation. In some embodiments, the orientation of the elements is reverse orientation. The sequences of individual expression cassette elements discussed herein (such as promoters, enhancers, transgenes, WPRE, poly(A), CMV intron and chimeric intron) can be any of the sequences of such elements provided herein or any sequences with at least, e.g., 75%, 80%, 85%, 90%, 95%, 97%, 98%, 99% or 100% sequence identity thereto.

In some embodiments, the expression cassette comprises two promoters described herein (with or without one or more enhancers described herein) driving the expression of two copies of a transgene (such as any transgene described herein). In some embodiments, the expression cassette comprises two promoters described herein (with or without one or more enhancers described herein) each promoter operably linked to one copy of a transgene (such as any transgene described herein). In some embodiments, the expression cassette comprises two promoters described herein, without any enhancer (e.g., without any enhancer described herein, e.g., without αMHCe and/or without ACTC1e) driving the expression of two copies of a transgene. In some embodiments, the expression cassette comprises two promoters described herein and one, two or more enhancers described herein (e.g., comprising an αMHCe and/or ACTC1e enhancer) driving the expression of two copies of a transgene. In some embodiments, the expression cassette comprises two promoters described herein and two enhancers described herein (e.g., comprising an αMHCe and/or ACTC1e enhancer) operably linked to two copies of a transgene, where each transgene is operably linked to one promoter and one enhancer. In some embodiments, the promoter is a cardiac-specific promoter, e.g., a human cTnT promoter (such as a short human promoter, hcTnTp) and/or a chicken cTnT promoter (such as ccTnTp). In the embodiments where two promoters are used, the two promoters can be the same or different. In some embodiments, where the two promoters drive the expression of two copies of a transgene (each promoter driving expression of one copy of the transgene), both promoters can be cardiac-specific promoters, either the same cardiac-specific promoters or different from each other. In some embodiments, both promoters can be human cTnT promoters (e.g., both can be a short human promoter, hcTnTp). In some embodiments, both promoters can be chicken cTnT promoters (such as ccTnT). In some embodiments, the two promoters are different, e.g., one is a human cTnT promoter (such as a short human cTnT promoter, hcTnTp) and one is a chicken cTnTpromoter (such as ccTnT). In some embodiments where two promoters and two transgenes are used, the two transgenes can be the same or different (such as the same or different variants of the same transgene). For example, the first copy of the transgene can be a non-codon-optimized polynucleotide sequence encoding a gene product, and the second copy of the transgene can be a codon-optimized polynucleotide sequence encoding the gene product. In some embodiments, both copies of the transgene used in an expression cassette are the same. In some of the embodiments where one or more enhancers are used, the enhancer is a cardiac-specific enhancer, e.g., αMHCe and/or ACTC1e. In some embodiments, two or more cardiac-specific enhancers are used, where the two or more of the enhancers can be the same or different (e.g., both or all αMHCe, both or all ACTC1e, or at least one αMHCe and at least one ACTC1e). In some embodiments, two cardiac-specific enhancers are used, where the two enhancers can be the same or different (e.g., both αMHCe, both ACTC1e, or one αMHCe and one ACTC1e). In some embodiments where two promoters are used, two cardiac-specific enhancers operably linked to the transgene are used as well, optionally wherein one enhancer is αMHCe and another is ACTC1e. In some embodiments, the expression cassette comprises at least two enhancers in the order of first αMHCe and then ACTC1e. In some embodiments, the expression cassette comprises at least two enhancers in the order of first ACTC1e and then αMHCe. In some embodiments where two promoters are used, one or more intron elements are also used. In some embodiments where two promoters are used, a CMV intron element is also used. In some embodiments where two promoters are used, a chimeric intron element (Chim int) is also used. In some embodiments where two promoters are used, a CMV intron and a chimeric intron (Chim int) are used. In some embodiments where two promoters are used, the order of the elements is as shown in any of the expression cassettes depicted in FIG. 7C. For example, (i) the order of the promoter and transgene elements can be as shown in FIG. 7C, (ii) the order of promoter, enhancer and transgene elements can be as shown in FIG. 7C, (iii) the order of promoter, transgene, WPRE and poly(A) elements can be as shown in FIG. 7C, optionally with or without enhancer elements being in the same order as shown in FIG. 7C, or (iv) the order of promoter, transgene, WPRE, poly(A), CMV intron (such as CMVint) and chimeric intron (such as Chim int) elements can be as shown in FIG. 7C, optionally with or without enhancer elements being in the same order as shown in FIG. 7C. In some embodiments where two promoters are used, the first promoter and the associated transgene (and, optionally an enhancer) are oriented in a forward 5′ to 3′ direction, and the second promoter and the associated transgene (and, optionally an enhancer) are oriented in a reverse direction. In some embodiments where two promoters are used, the first promoter and the associated transgene (and, optionally an enhancer) are oriented in reverse relative to 5′ to 3′ direction, and the second promoter and the associated transgene (and, optionally an enhancer) are oriented in a forward direction. In some embodiments where two promoters are used, both promoters and the associated transgenes (and, optionally an enhancer) are oriented in a forward 5′ to 3′ direction. In some embodiments where two promoters are used, both promoters and the associated transgenes (and, optionally an enhancer) are oriented in reverse relative to the 5′ to 3′ direction. In some embodiments where two promoters are used, the orientation of any of the elements (such forward or reverse orientation in 5′ to 3′ direction) is as shown in any of the expression cassettes depicted in FIG. 7C. In some embodiments where two promoters are used, the orientation of promoters, enhancers if any, transgenes, WPRE, poly(A), CMV intron and chimeric intron elements (such forward or reverse orientation in 5′ to 3′ direction) is as shown in any of the expression cassettes depicted in FIG. 7C. The sequences of individual expression cassette elements discussed herein (such as promoters, enhancers, transgenes, WPRE, poly(A), CMV intron and chimeric intron) can be any of the sequences of such elements provided herein or any sequences with at least, e.g., 75%, 80%, 85%, 90%, 95%, 97%, 98%, 99% or 100% sequence identity thereto.

Expression cassette sequences of the disclosure can be found, without limitation, in Table 1. In some embodiments, the expression cassette comprises about 3.2 kilobases (kb), 3.3 kb, 3.4 kb, 3.5 kb, 3.6 kb, 3.7 kb, or less. In some embodiments, the expression cassette comprises about 1.9 kb, 2.1 kb, 2.2 kb, 2.3 kb, 2.4 kb, 2.5 kb, 2.6 kb, 2.7 kb, 2.8 kb, 2.9 kb, 3.0 kb, 3.1 kb, 3.2 kb, or more.

In some embodiments, the expression cassette comprises a polynucleotide sequence that shares at least 75%, 80%, 90%, 95%, 96%, 97%, 98%, 99%, or 100% identity to SEQ ID NOs: 20-24 and SEQ ID NOs: 45-63. In some embodiments, the expression cassette comprises a polynucleotide sequence that shares at least 75%, 80%, 90%, 95%, 96%, 97%, 98%, 99%, or 100% identity to SEQ ID NOs: 64-75. In some embodiments, the expression cassette comprises a polynucleotide sequence that shares at least 75%, 80%, 90%, 95%, 96%, 97%, 98%, 99%, or 100% identity to SEQ ID NO: 61. In some embodiments, the expression cassette comprises SEQ ID NO: 61. In some embodiments, the expression cassette comprises a polynucleotide sequence that shares at least 75%, 80%, 90%, 95%, 96%, 97%, 98%, 99%, or 100% identity to SEQ ID NO: 62. In some embodiments, the expression cassette comprises SEQ ID NO: 62. In some embodiments, the expression cassette comprises a polynucleotide sequence that shares at least 75%, 80%, 90%, 95%, 96%, 97%, 98%, 99%, or 100% identity to SEQ ID NO: 63. In some embodiments, the expression cassette comprises SEQ ID NO: 63. In some embodiments, the expression cassette comprises a polynucleotide sequence that shares at least 75%, 80%, 90%, 95%, 96%, 97%, 98%, 99%, or 100% identity to SEQ ID NO: 49. In some embodiments, the expression cassette comprises a polynucleotide sequence that shares at least 75%, 80%, 90%, 95%, 96%, 97%, 98%, 99%, or 100% identity to SEQ ID NO: 51. In some embodiments, the expression cassette comprises a polynucleotide sequence that shares at least 75%, 80%, 90%, 95%, 96%, 97%, 98%, 99%, or 100% identity to SEQ ID NO: 55. In some embodiments, the expression cassette comprises a polynucleotide sequence that shares at least 75%, 80%, 90%, 95%, 96%, 97%, 98%, 99%, or 100% identity to SEQ ID NO: 56. In some embodiments, the expression cassette comprises a polynucleotide sequence that shares at least 75%, 80%, 90%, 95%, 96%, 97%, 98%, 99%, or 100% identity to SEQ ID NO: 57. In some embodiments, the expression cassette comprises a polynucleotide sequence that shares at least 75%, 80%, 90%, 95%, 96%, 97%, 98%, 99%, or 100% identity to SEQ ID NO: 58. In some embodiments, the expression cassette comprises a polynucleotide sequence that shares at least 75%, 80%, 90%, 95%, 96%, 97%, 98%, 99%, or 100% identity to SEQ ID NO: 59. In some embodiments, the expression cassette comprises a polynucleotide sequence that shares at least 75%, 80%, 90%, 95%, 96%, 97%, 98%, 99%, or 100% identity to SEQ ID NO: 60. In some embodiments, the expression cassette comprises a polynucleotide sequence that shares at least 75%, 80%, 90%, 95%, 96%, 97%, 98%, 99%, or 100% identity to SEQ ID NO: 67. In some embodiments, the expression cassette comprises a polynucleotide sequence that shares at least 75%, 80%, 90%, 95%, 96%, 97%, 98%, 99%, or 100% identity to SEQ ID NO: 69. In some embodiments, the expression cassette comprises a polynucleotide sequence that shares at least 75%, 80%, 90%, 95%, 96%, 97%, 98%, 99%, or 100% identity to SEQ ID NO: 74. In some embodiments, the expression cassette comprises a polynucleotide sequence that shares at least 75%, 80%, 90%, 95%, 96%, 97%, 98%, 99%, or 100% identity to SEQ ID NO: 75. In some of these embodiments, the sequence encoding DWORF (the DWORF open reading frame) can be replaced by a sequence encoding another polypeptide described herein, and the sequence identity referenced above does not take into account the part of the polynucleotide sequence encoding DWORF (the DWORF open reading frame).

In some embodiments, the transgene in the expression cassette encodes a polypeptide for use in treating or preventing a heart disease or disorder. In some embodiments, the transgene in the expression cassette encodes a polypeptide selected from: DWORF, junctophilin (e.g., JPH2), BAG family molecular chaperone regulator 3 (BAG3), alpha-crystallin B chain (CRYAB), LMNA (such as Lamin A and Lamin C isoforms), troponin I type 3 (TNNI3), phospholamban (PLN), lysosomal-associated membrane protein 2 (LAMP2, such as LAMP2a, LAMP2b and LAMP2c isoforms), desmoplakin (DSP, such as DPI and DPII isoforms), desmoglein 2 (DSG2), and junction plakoglobin (JUP), or a variant of any of these polypeptides (e.g., having at least 75%, at least 85%, at least 95%, at least 97% or at least 99% sequence identity thereto). In some embodiments, the transgene in the expression cassette encodes DWORF (or a variant thereof). In some embodiments, the transgene in the expression cassette encodes JPH2 (e.g., a full-length JPH2 or an N-terminal fragment of JPH2) (or a variant thereof). In some embodiments, the transgene in the expression cassette encodes BAG3 (or a variant thereof). In some embodiments, the transgene in the expression cassette encodes CRYAB (or a variant thereof). In some embodiments, the transgene in the expression cassette encodes LMNA Lamin A isoform (or a variant thereof). In some embodiments, the transgene in the expression cassette encodes LMNA Lamin C isoform (or a variant thereof). In some embodiments, the transgene in the expression cassette encodes TNNI3 (or a variant thereof). In some embodiments, the transgene in the expression cassette encodes PLN (or a variant thereof). In some embodiments, the transgene in the expression cassette encodes LAMP2a (or a variant thereof). In some embodiments, the transgene in the expression cassette encodes LAMP2b (or a variant thereof). In some embodiments, the transgene in the expression cassette encodes LAMP2c (or a variant thereof). In some embodiments, the transgene in the expression cassette encodes DSP DPI isoform (or a variant thereof). In some embodiments, the transgene in the expression cassette encodes DSP DPII isoform (or a variant thereof). In some embodiments, the transgene in the expression cassette encodes DSG2 (or a variant thereof). In some embodiments, the transgene in the expression cassette encodes JUP (or a variant thereof). In some embodiments, the transgene in the expression cassette encodes a human polypeptide (such as any human polypeptide described herein).

In some embodiments, the expression cassettes described herein lead to cardiac cell-specific expression of a transgene. In some embodiments, the expression cassettes described herein lead to cardiomyocyte-specific expression of a transgene. In some embodiments, the expression cassettes described herein allow high expression of a transgene in a cardiac cell (e.g., a cardiomyocyte) and low or no expression in other cells (e.g., low or no expression in liver cells, low or no expression in muscle cells except for muscle cells of the heart, low or no expression in cardiac fibroblasts). In some embodiments, the expression cassettes described herein allow high expression of a transgene in heart tissue of a subject (e.g., in human heart). In some embodiments, the expression cassettes described herein allow no or low expression of a transgene in tissues of a subject other than the heart (e.g., in liver or in muscles except those of the heart). “High” and “low” can be relative to each other, for example, the expression of a transgene in cardiac cells (e.g., cardiomyocytes) and/or heart tissue can be at least 2 fold, 5 fold, 10 fold, 15 fold, 20 fold, 50 fold, 100 fold, 150 fold, or 200 fold higher than its expression in other cells and tissues (e.g., liver, muscle except for the heart).

TABLE 1
Illustrative Expression Cassette Sequences

| Expression Cassette | Sequence |
|---|---|
| pCR-MD1 | CTGCGCGCTCGCTCGCTCACTGAGGCCGCCCGGGCAAAGC |
| distinct sequence elements | CCGGGCGTCGGGCGACCTTTGGTCGCCCGGCCTCAGTGAG |
| are indicated in bold, | CGAGCGAGCGCGCAGAGAGGGAGTGGCCAACTCCATCACT |
| starting with ITR, hcTnTp, | AGGGGTTCCTTGTAGTTAATGATTAACCCGCCATGCTACT |
| DWORF (which is also | TATCTACGTAGCCATGCTCTAGGAAGATCGGAATTCGCCC |
| underlined), WPRE, polyA | TTAAGGTCATGGAGAAGACCCACCTTGCAGATGTCCTCAC |
| (hGHpA), ITR | TGGGGCTGGCAGAGCCGGCAACCTGCCTAAGGCTGCTCAG |
| | TCCATTAGGAGCCAGTAGCCTGGAAGATGTCTTTACCCCC |
| | AGCATCAGTTCAAGTGGAGCAGCACATAACTCTTGCCCTC |
| | TGCCTTCCAAGATTCTGGTGCTGAGACTTATGGAGTGTCT |
| | TGGAGGTTGCCTTCTGCCCCCCAACCCTGCTCCCAGCTGG |
| | CCCTCCCAGGCCTGGGTTGCTGGCCTCTGCTTTATCAGGA |
| | TTCTCAAGAGGGACAGCTGGTTTATGTTGCATGACTGTTC |
| | CCTGCATATCTGCTCTGGTTTTAAATAGCTTATCTGAGCA |
| | GCTGGAGGACCACATGGGCTTATATGGCGTGGGGTACATG |
| | TTCCTGTAGCCTTGTCCCTGGCACCTGCCAAAATAGCAGC |
| | CAACACCCCCCACCCCCACCGCCATCCCCCTGCCCCACCC |
| | GTCCCCTGTCGCACATTCCTCCCTCCGCAGGGCTGGCTCA |
| | CCAGGCCCCAGCCCACATGCCTGCTTAAAGCCCTCTCCAT |
| | CCTCTGCCTCACCCAGTCCCCGCTGAGACTGAGCAGACGC |
| | CTCCAGCGGCCGCCCGCCACCATGGCTGAGAAAGAGTCAA |
| | CATCACCACACCTCATGGTTCCCATTCTTCTCCTGGTTGG |
| | ATGGATTGTAGGCTGCATCATCGTTATTTACATTGTCTTC |
| | TTCTAAAAGCTTTGGATCCAATCAACCTCTGGATTACAAA |
| | ATTTGTGAAAGATTGACTGGTATTCTTAACTATGTTGCTC |
| | CTTTTACGCTATGTGGATACGCTGCTTTAATGCCTTTGTA |
| | TCATGCTATTGCTTCCCGTATGGCTTTCATTTTCTCCTCC |
| | TTGTATAAATCCTGGTTGCTGTCTCTTTATGAGGAGTTGT |
| | GGCCCGTTGTCAGGCAACGTGGCGTGGTGTGCACTGTGTT |
| | TGCTGACGCAACCCCCACTGGTTGGGGCATTGCCACCACC |
| | TGTCAGCTCCTTTCCGGGACTTTCGCTTTCCCCCTCCCTA |
| | TTGCCACGGCGGAACTCATCGCCGCCTGCCTTGCCCGCTG |
| | CTGGACAGGGGCTCGGCTGTTGGGCACTGACAATTCCGTG |
| | GTGTTGTCGGGGAAATCATCGTCCTTTCCTTGGCTGCTCG |
| | CCTGTGTTGCCACCTGGATTCTGCGCGGGACGTCCTTCTG |
| | CTACGTCCCTTCGGCCCTCAATCCAGCGGACCTTCCTTCC |
| | CGCGGCCTGCTGCCGGCTCTGCGGCCTCTTCCGCGTCTTC |
| | GAGATCTGCCTCGACTGTGCCTTCTAGTTGCCAGCCATCT |
| | GTTGTTTGCCCCTCCCCCGTGCCTTCCTTGACCCTGGAAG |
| | GTGCCACTCCCACTGTCCTTTCCTAATAAAATGAGGAAAT |
| | TGCATCGCATTGTCTGAGTAGGTGTCATTCTATTCTGGGG |
| | GGTGGGGTGGGGCAGGACAGCAAGGGGGAGGATTGGGAAG |
| | ACAATAGCAGGCATGCTGGGGACTCGAGTTAAGGGCGAAT |
| | TCCCGATTAGGATCTTCCTAGAGCATGGCTACGTAGATAA |
| | GTAGCATGGCGGGTTAATCATTAACTACAAGGAACCCCTA |
| | GTGATGGAGTTGGCCACTCCCTCTCTGCGCGCTCGCTCGC |
| | TCACTGAGGCCGGGCGACCAAAGGTCGCCCGACGCCCGGG |
| | CTTTGCCCGGGCGGCCTCAGTGAGCGAGCGAGCGCGCAG |
| | (SEQ ID NO: 20) |
| pCR-MD2 | CTGCGCGCTCGCTCGCTCACTGAGGCCGCCCGGGCAAAGC |
| | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGAGAGGACCCTTTCAAGGACATTAGTGGTGGAGGCA |
| | GCATAGTAGCTCCCAAGGCAGAGGGATTGAGAGAAGAGTT |
| | TGAGGACTGGGAAGGCGGGACACATGATTGGGTGATGGGA |
| | GAAGGGGGCAGAGAATAGCGAGATTGCTTTCTTTGCCCAC |
| | GGAGAAACAGAGGAGTGTGGATCATGAATGGGCAAGATCT |
| | TTAAGTGCCAGGGGGGGTCATGGAGGAGGGGAGGGCCTGC |
| | TCCAGAGGAGGACCATTCCTGCTTCAGAGCCAAGCAGGAC |
| | CTAGGCTGTGAAGATTCGGAGAAAGAGATGGAGGGGAGAG |
| | TCAGCTCAGCTGCTTACTGGCTTGCTTTCCTCCTGTCTCT |
| | TTCATTTTCATAATCTACCAAACCCTGCAATGGGCCAGCC |
| | TTGAACATACAAGTGCATGTGCATGGTCAGACACAGGCAA |
| | GCAAGCAAGACCCCTAGGCCTGACCTATGCATCTGCAATC |
| | TAGTAGGTTTAGCAGATCATAGCCCCGCACTGCTTGATTT |
| | TAAAGCCGTTAGGGGATGACCTTTGACAGTCCGCATCACC |
| | CCTCTCACACAACGAGCGCCTGTTCAAGGTTCTTGACTGG |
| | AAGTTCTACCTTGTATCTGGCCTCCTGTAGCAGTTTCAGT |
| | CCATTCCCTGTGAGGAGGGTGTGCCACATGGCTTTGGGGG |
| | TCATGGAGAAGACCCACCTTGCAGATGTCCTCACTGGGGC |
| | TGGCAGAGCCGGCAACCTGCCTAAGGCTGCTCAGTCCATT |
| | AGGAGCCAGTAGCCTGGAAGATGTCTTTACCCCCAGCATC |
| | AGTTCAAGTGGAGCAGCACATAACTCTTGCCCTCTGCCTT |
| | CCAAGATTCTGGTGCTGAGACTTATGGAGTGTCTTGGAGG |
| | TTGCCTTCTGCCCCCCAACCCTGCTCCCAGCTGGCCCTCC |
| | CAGGCCTGGGTTGCTGGCCTCTGCTTTATCAGGATTCTCA |
| | AGAGGGACAGCTGGTTTATGTTGCATGACTGTTCCCTGCA |
| | TATCTGCTCTGGTTTTAAATAGCTTATCTGAGCAGCTGGA |
| | GGACCACATGGGCTTATATGGCGTGGGGTACATGTTCCTG |
| | TAGCCTTGTCCCTGGCACCTGCCAAAATAGCAGCCAACAC |
| | CCCCCACCCCCACCGCCATCCCCCTGCCCCACCCGTCCCC |
| | TGTCGCACATTCCTCCCTCCGCAGGGCTGGCTCACCAGGC |
| | CCCAGCCCACATGCCTGCTTAAAGCCCTCTCCATCCTCTG |
| | CCTCACCCAGTCCCCGCTGAGACTGAGCAGACGCCTCCAG |
| | GATCTGTCGGCAGCTGCTGTTCTGAGGTAAGGCTCGGGCA |
| | GGGCTCTGGGGAAGAGGAGAGCAGAGAATGGACGGGGAGA |
| | TGTGAGGGTCTTGGGCCCTGGCATATTTACCCAGAGTCTG |
| | CCTGTGTCCGCAGAAGTCCATGGCCCCTCCTGGTGGAGGC |
| | CACACTTCAGAGGACAGGTTGCCAGGTCTGGGCTCCAAGA |
| | TTGGTACAATAGAGCAGAGAGAGGAGTCGCTGCGACGCTG |
| | CCTTCGCCCCGTGCCCCGCTCCGCCGCCGCCTCGCGCCGC |
| | CCGCCCCGGCTCTGACTGACCGCGTTACTCCCACAGGTGA |
| | GCGGGCGGGACGGCCCTTCTCCTCCGGGCTGTAATTAGCG |
| | CTTGGTTTAATGACGGCTTGTTTCTTTTCTGTGGCTGCGT |
| | GAAAGCCTTGAGGGGCTCCGGGAGCTAGAGCCTCTGCTAA |
| | CCATGTTCATGCCTTCTTCTTTTTCCTACAGCTCCTGGGC |
| | AACGTGCTGGTTATTGTGCTGTCTCATCATTTTGGCAAAG |
| | AATTCCCAATCGATACCCAATCGATACAGATCTAGCGGCC |
| | GCGCCGCCACCATGGCTGAGAAAGAGTCAACATCACCACA |
| | CCTCATGGTTCCCATTCTTCTCCTGGTTGGATGGATTGTA |
| | GGCTGCATCATCGTTATTTACATTGTCTTCTTCTAACCAG |
| | AGGTTGATTGGATCCAAGCTTTGGATCCAATGGATCCAAT |
| | CAACCTCTGGATTACAAAATTTGTGAAAGATTGACTGGTA |
| | TTCTTAACTATGTTGCTCCTTTTACGCTATGTGGATACGC |
| | TGCTTTAATGCCTTTGTATCATGCTATTGCTTCCCGTATG |
| | GCTTTCATTTTCTCCTCCTTGTATAAATCCTGGTTGCTGT |
| | CTCTTTATGAGGAGTTGTGGCCCGTTGTCAGGCAACGTGG |
| | CGTGGTGTGCACTGTGTTTGCTGACGCAACCCCCACTGGT |
| | TGGGGCATTGCCACCACCTGTCAGCTCCTTTCCGGGACTT |
| | TCGCTTTCCCCCTCCCTATTGCCACGGCGGAACTCATCGC |
| | CGCCTGCCTTGCCCGCTGCTGGACAGGGGCTCGGCTGTTG |
| | GGCACTGACAATTCCGTGGTGTTGTCGGGGAAATCATCGT |
| | CCTTTCCTTGGCTGCTCGCCTGTGTTGCCACCTGGATTCT |
| | GCGCGGGACGTCCTTCTGCTACGTCCCTTCGGCCCTCAAT |
| | CCAGCGGACCTTCCTTCCCGCGGCCTGCTGCCGGCTCTGC |
| | GGCCTCTTCCGCGTCTTCGAGATCTGCCTCGACTGTGCCT |
| | TCTAGTTGCCAGCCATCTGTTGTTTGCCCCTCCCCCGTGC |
| | CTTCCTTGACCCTGGAAGGTGCCACTCCCACTGTCCTTTC |
| | CTAATAAAATGAGGAAATTGCATCGCATTGTCTGAGTAGG |
| | TGTCATTCTATTCTGGGGGGTGGGGTGGGGCAGGACAGCA |
| | AGGGGGAGGATTGGGAAGACAATAGCAGGCATGCTGGGGA |
| | CTCGAGTTAAGGGCGAATTCCCGATTAGGATCTTCCTAGA |
| | GCATGGCTACGTAGATAAGTAGCATGGCGGGTTAATCATT |
| | AACTACAAGGAACCCCTAGTGATGGAGTTGGCCACTCCCT |
| | CTCTGCGCGCTCGCTCGCTCACTGAGGCCGGGCGACCAAA |
| | GGTCGCCCGACGCCCGGGCTTTGCCCGGGCGGCCTCAGTG |
| | AGCGAGCGAGCGCGCAG (SEQ ID NO: 21) |
| pCR-HD1 | CTGCGCGCTCGCTCGCTCACTGAGGCCGCCCGGGCAAAGC |
| | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGGTCATGGAGAAGACCCACCTTGCAGATGTCCTCAC |
| | TGGGGCTGGCAGAGCCGGCAACCTGCCTAAGGCTGCTCAG |
| | TCCATTAGGAGCCAGTAGCCTGGAAGATGTCTTTACCCCC |
| | AGCATCAGTTCAAGTGGAGCAGCACATAACTCTTGCCCTC |
| | TGCCTTCCAAGATTCTGGTGCTGAGACTTATGGAGTGTCT |
| | TGGAGGTTGCCTTCTGCCCCCCAACCCTGCTCCCAGCTGG |
| | CCCTCCCAGGCCTGGGTTGCTGGCCTCTGCTTTATCAGGA |
| | TTCTCAAGAGGGACAGCTGGTTTATGTTGCATGACTGTTC |
| | CCTGCATATCTGCTCTGGTTTTAAATAGCTTATCTGAGCA |
| | GCTGGAGGACCACATGGGCTTATATGGCGTGGGGTACATG |
| | TTCCTGTAGCCTTGTCCCTGGCACCTGCCAAAATAGCAGC |
| | CAACACCCCCCACCCCCACCGCCATCCCCCTGCCCCACCC |
| | GTCCCCTGTCGCACATTCCTCCCTCCGCAGGGCTGGCTCA |
| | CCAGGCCCCAGCCCACATGCCTGCTTAAAGCCCTCTCCAT |
| | CCTCTGCCTCACCCAGTCCCCGCTGAGACTGAGCAGACGC |
| | CTCCAGCGGCCGCCCGCCACCATGGCAGAGAAGGCTGGAA |
| | GCACTTTCTCTCACCTGCTCGTGCCGATTTTGCTTTTGAT |
| | TGGGTGGATAGTTGGCTGTATCATAATGATCTACGTTGTC |
| | TTTTCATAGAAGCTTTGGATCCAATCAACCTCTGGATTAC |
| | AAAATTTGTGAAAGATTGACTGGTATTCTTAACTATGTTG |
| | CTCCTTTTACGCTATGTGGATACGCTGCTTTAATGCCTTT |
| | GTATCATGCTATTGCTTCCCGTATGGCTTTCATTTTCTCC |
| | TCCTTGTATAAATCCTGGTTGCTGTCTCTTTATGAGGAGT |
| | TGTGGCCCGTTGTCAGGCAACGTGGCGTGGTGTGCACTGT |
| | GTTTGCTGACGCAACCCCCACTGGTTGGGGCATTGCCACC |
| | ACCTGTCAGCTCCTTTCCGGGACTTTCGCTTTCCCCCTCC |
| | CTATTGCCACGGCGGAACTCATCGCCGCCTGCCTTGCCCG |
| | CTGCTGGACAGGGGCTCGGCTGTTGGGCACTGACAATTCC |
| | GTGGTGTTGTCGGGGAAATCATCGTCCTTTCCTTGGCTGC |
| | TCGCCTGTGTTGCCACCTGGATTCTGCGCGGGACGTCCTT |
| | CTGCTACGTCCCTTCGGCCCTCAATCCAGCGGACCTTCCT |
| | TCCCGCGGCCTGCTGCCGGCTCTGCGGCCTCTTCCGCGTC |
| | TTCGAGATCTGCCTCGACTGTGCCTTCTAGTTGCCAGCCA |
| | TCTGTTGTTTGCCCCTCCCCCGTGCCTTCCTTGACCCTGG |
| | AAGGTGCCACTCCCACTGTCCTTTCCTAATAAAATGAGGA |
| | AATTGCATCGCATTGTCTGAGTAGGTGTCATTCTATTCTG |
| | GGGGGTGGGGTGGGGCAGGACAGCAAGGGGGAGGATTGGG |
| | AAGACAATAGCAGGCATGCTGGGGACTCGAGTTAAGGGCG |
| | AATTCCCGATTAGGATCTTCCTAGAGCATGGCTACGTAGA |
| | TAAGTAGCATGGCGGGTTAATCATTAACTACAAGGAACCC |
| | CTAGTGATGGAGTTGGCCACTCCCTCTCTGCGCGCTCGCT |
| | CGCTCACTGAGGCCGGGCGACCAAAGGTCGCCCGACGCCC |
| | GGGCTTTGCCCGGGCGGCCTCAGTGAGCGAGCGAGCGCGC |
| | AG (SEQ ID NO: 22) |
| pCR-HD2 | CTGCGCGCTCGCTCGCTCACTGAGGCCGCCCGGGCAAAGC |
| | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGAGAGGACCCTTTCAAGGACATTAGTGGTGGAGGCA |
| | GCATAGTAGCTCCCAAGGCAGAGGGATTGAGAGAAGAGTT |
| | TGAGGACTGGGAAGGCGGGACACATGATTGGGTGATGGGA |
| | GAAGGGGGCAGAGAATAGCGAGATTGCTTTCTTTGCCCAC |
| | GGAGAAACAGAGGAGTGTGGATCATGAATGGGCAAGATCT |
| | TTAAGTGCCAGGGGGGGTCATGGAGGAGGGGAGGGCCTGC |
| | TCCAGAGGAGGACCATTCCTGCTTCAGAGCCAAGCAGGAC |
| | CTAGGCTGTGAAGATTCGGAGAAAGAGATGGAGGGGAGAG |
| | TCAGCTCAGCTGCTTACTGGCTTGCTTTCCTCCTGTCTCT |
| | TTCATTTTCATAATCTACCAAACCCTGCAATGGGCCAGCC |
| | TTGAACATACAAGTGCATGTGCATGGTCAGACACAGGCAA |
| | GCAAGCAAGACCCCTAGGCCTGACCTATGCATCTGCAATC |
| | TAGTAGGTTTAGCAGATCATAGCCCCGCACTGCTTGATTT |
| | TAAAGCCGTTAGGGGATGACCTTTGACAGTCCGCATCACC |
| | CCTCTCACACAACGAGCGCCTGTTCAAGGTTCTTGACTGG |
| | AAGTTCTACCTTGTATCTGGCCTCCTGTAGCAGTTTCAGT |
| | CCATTCCCTGTGAGGAGGGTGTGCCACATGGCTTTGGGGG |
| | TCATGGAGAAGACCCACCTTGCAGATGTCCTCACTGGGGC |
| | TGGCAGAGCCGGCAACCTGCCTAAGGCTGCTCAGTCCATT |
| | AGGAGCCAGTAGCCTGGAAGATGTCTTTACCCCCAGCATC |
| | AGTTCAAGTGGAGCAGCACATAACTCTTGCCCTCTGCCTT |
| | CCAAGATTCTGGTGCTGAGACTTATGGAGTGTCTTGGAGG |
| | TTGCCTTCTGCCCCCCAACCCTGCTCCCAGCTGGCCCTCC |
| | CAGGCCTGGGTTGCTGGCCTCTGCTTTATCAGGATTCTCA |
| | AGAGGGACAGCTGGTTTATGTTGCATGACTGTTCCCTGCA |
| | TATCTGCTCTGGTTTTAAATAGCTTATCTGAGCAGCTGGA |
| | GGACCACATGGGCTTATATGGCGTGGGGTACATGTTCCTG |
| | TAGCCTTGTCCCTGGCACCTGCCAAAATAGCAGCCAACAC |
| | CCCCCACCCCCACCGCCATCCCCCTGCCCCACCCGTCCCC |
| | TGTCGCACATTCCTCCCTCCGCAGGGCTGGCTCACCAGGC |
| | CCCAGCCCACATGCCTGCTTAAAGCCCTCTCCATCCTCTG |
| | CCTCACCCAGTCCCCGCTGAGACTGAGCAGACGCCTCCAG |
| | GATCTGTCGGCAGCTGCTGTTCTGAGGTAAGGCTCGGGCA |
| | GGGCTCTGGGGAAGAGGAGAGCAGAGAATGGACGGGGAGA |
| | TGTGAGGGTCTTGGGCCCTGGCATATTTACCCAGAGTCTG |
| | CCTGTGTCCGCAGAAGTCCATGGCCCCTCCTGGTGGAGGC |
| | CACACTTCAGAGGACAGGTTGCCAGGTCTGGGCTCCAAGA |
| | TTGGTACAATAGAGCAGAGAGAGGAGTCGCTGCGACGCTG |
| | CCTTCGCCCCGTGCCCCGCTCCGCCGCCGCCTCGCGCCGC |
| | CCGCCCCGGCTCTGACTGACCGCGTTACTCCCACAGGTGA |
| | GCGGGCGGGACGGCCCTTCTCCTCCGGGCTGTAATTAGCG |
| | CTTGGTTTAATGACGGCTTGTTTCTTTTCTGTGGCTGCGT |
| | GAAAGCCTTGAGGGGCTCCGGGAGCTAGAGCCTCTGCTAA |
| | CCATGTTCATGCCTTCTTCTTTTTCCTACAGCTCCTGGGC |
| | AACGTGCTGGTTATTGTGCTGTCTCATCATTTTGGCAAAG |
| | AATTCCCAATCGATACCCAATCGATACAGATCTAGCGGCC |
| | GCGCCGCCACCATGGCAGAGAAGGCTGGAAGCACTTTCTC |
| | TCACCTGCTCGTGCCGATTTTGCTTTTGATTGGGTGGATA |
| | GTTGGCTGTATCATAATGATCTACGTTGTCTTTTCATAGC |
| | CAGAGGTTGATTGGATCCAAGCTTTGGATCCAATGGATCC |
| | AATCAACCTCTGGATTACAAAATTTGTGAAAGATTGACTG |
| | GTATTCTTAACTATGTTGCTCCTTTTACGCTATGTGGATA |
| | CGCTGCTTTAATGCCTTTGTATCATGCTATTGCTTCCCGT |
| | ATGGCTTTCATTTTCTCCTCCTTGTATAAATCCTGGTTGC |
| | TGTCTCTTTATGAGGAGTTGTGGCCCGTTGTCAGGCAACG |
| | TGGCGTGGTGTGCACTGTGTTTGCTGACGCAACCCCCACT |
| | GGTTGGGGCATTGCCACCACCTGTCAGCTCCTTTCCGGGA |
| | CTTTCGCTTTCCCCCTCCCTATTGCCACGGCGGAACTCAT |
| | CGCCGCCTGCCTTGCCCGCTGCTGGACAGGGGCTCGGCTG |
| | TTGGGCACTGACAATTCCGTGGTGTTGTCGGGGAAATCAT |
| | CGTCCTTTCCTTGGCTGCTCGCCTGTGTTGCCACCTGGAT |
| | TCTGCGCGGGACGTCCTTCTGCTACGTCCCTTCGGCCCTC |
| | AATCCAGCGGACCTTCCTTCCCGCGGCCTGCTGCCGGCTC |
| | TGCGGCCTCTTCCGCGTCTTCGAGATCTGCCTCGACTGTG |
| | CCTTCTAGTTGCCAGCCATCTGTTGTTTGCCCCTCCCCCG |
| | TGCCTTCCTTGACCCTGGAAGGTGCCACTCCCACTGTCCT |
| | TTCCTAATAAAATGAGGAAATTGCATCGCATTGTCTGAGT |
| | AGGTGTCATTCTATTCTGGGGGGTGGGGTGGGGCAGGACA |
| | GCAAGGGGGAGGATTGGGAAGACAATAGCAGGCATGCTGG |
| | GGACTCGAGTTAAGGGCGAATTCCCGATTAGGATCTTCCT |
| | AGAGCATGGCTACGTAGATAAGTAGCATGGCGGGTTAATC |
| | ATTAACTACAAGGAACCCCTAGTGATGGAGTTGGCCACTC |
| | CCTCTCTGCGCGCTCGCTCGCTCACTGAGGCCGGGCGACC |
| | AAAGGTCGCCCGACGCCCGGGCTTTGCCCGGGCGGCCTCA |
| | GTGAGCGAGCGAGCGCGCAG (SEQ ID NO: 23) |
| | CTGCGCGCTCGCTCGCTCACTGAGGCCGCCCGGGCAAAGC |
| | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACCAGGGTAATGGGGATCCTCTAGAACTATAGCTA |
| | GAATTCGCCCTTACGGGCCCCCCCTCGAGGTCGGGATAAA |
| | AGCAGTCTGGGCTTTCACATGACAGCATCTGGGGCTGCGG |
| | CAGAGGGTCGGGTCCGAAGCGCTGCCTTATCAGCGTCCCC |
| | AGCCCTGGGAGGTGACAGCTGGCTGGCTTGTGTCAGCCCC |
| | TCGGGCACTCACGTATCTCCGTCCGACGGGTTTAAAATAG |
| | CAAAACTCTGAGGCCACACAATAGCTTGGGCTTATATGGG |
| | CTCCTGTGGGGGAAGGGGGAGCACGGAGGGGGCCGGGGCC |
| | GCTGCTGCCAAAATAGCAGCTCACAAGTGTTGCATTCCTC |
| | TCTGGGCGCCGGGCACATTCCTGCTGGCTCTGCCCGCCCC |
| | GGGGTGGGCGCCGGGGGGACCTTAAAGCCTCTGCCCCCCA |
| | AGGAGCCCTTCCCAGACAGCCGCCGGCACCCACCGCTCCG |
| | TGGGACGATCCCCGAAGCTCTAGAGCTTTATTGCGGTAGT |
| | TTATCACAGTTAAATTGCTAACGCAGTCAGTGCTTCTGAC |
| | ACAACAGTCTCGAACTTAAGCTGCAGAAGTTGGTCGTGAG |
| | GCACTGGGCAGGTAAGTATCAAGGTTACAAGACAGGTTTA |
| | AGGAGACCAATAGAAACTGGGCTTGTCGAGACAGAGAAGA |
| | CTCTTGCGTTTCTGATAGGCACCTATTGGTCTTACTGACA |
| | TCCACTTTGCCTTTCTCTCCACAGGTGTCCACTCCCAGTT |
| | CAATTACAGCTCTTAAGGCTAGAGTACTTAATACGACTCA |
| | CTATAGGCTAGCCGCCACCATGGCTGAGAAAGAGTCAACA |
| | TCACCACACCTCATGGTTCCCATTCTTCTCCTGGTTGGAT |
| | GGATTGTAGGCTGCATCATCGTTATTTACATTGTCTTCTT |
| | CTAACGGCCGCGCGGATCCAGACATGATAAGATACATTGA |
| | TGAGTTTGGACAAACCACAACTAGAATGCAGTGAAAAAAA |
| | TGCTTTATTTGTGAAATTTGTGATGCTATTGCTTTATTTG |
| | TAACCATTATAAGCTGCAATAAACAAGTTAACAACAACAA |
| | TTGCATTCATTTTATGTTTCAGGTTCAGGGGGAGGTGTGG |
| | GAGGTTTTTTAGTCGACCCGGGCGGCCTCGAGGACGGGGT |
| | GAACTACGCCTGAGGATCCGATCTTTTTCCCTCTGCCAAA |
| | AATTATGGGGACATCATGAAGCCCCTTGAGCATCTGACTT |
| | CTGGCTAATAAAGGAAATTTATTTTCATTGCAATAGTGTG |
| | TTGGAATTTTTTGTGTCTCTCACTCGGAAGCAATTCGTTG |
| | ATCTGAATTTCGACCACCCATAATACCCATTACCCTGGTA |
| | GATAAGTAGCATGGCGGGTTAATCATTAACTACAAGGAAC |
| | CCCTAGTGATGGAGTTGGCCACTCCCTCTCTGCGCGCTCG |
| | CTCGCTCACTGAGGCCGGGCGACCAAAGGTCGCCCGACGC |
| | CCGGGCTTTGCCCGGGCGGCCTCAGTGAGCGAGCGAGCGC |
| | GCAG (SEQ ID NO: 24) |
| pHZ15 | CTGCGCGCTCGCTCGCTCACTGAGGCCGCCCGGGCAAAGC |
| | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGAACTGGCCTGCCCGAGACCAAACGTGCGGAACGTA |
| | GTTAAGTGTTAGAGGTAGGATTTGAAGCCTGTCGATCATT |
| | CTGATTCTCCTTTTCTCTACGTCTGCTTCCTGTCAATGGG |
| | CATCCTCACTGTCAAATGCAGATGGTACAGCAGGGCTTGG |
| | TCTCAGCCAGGCAGGCCTCTCCCCAGTCTCCATGGCTCAG |
| | CTGTCCAGCAGTTTCATCCCTAGACCATCCCAAACATGGT |
| | TGAGAAGCTCTGAGGGGAGGACCCAGCACTGCCCGGCCCC |
| | TGAAGATAATCAGCAGTCCTGCTCAGCATATCAATCCAAG |
| | CCCACTCTAGACAGAGATGCCGGTGCCCAGTTTTCTATTT |
| | TTAACTGGTGTGAACTGAAGGAAAAGCACAGCATTAGAAG |
| | TCCAAGCAGTCATGGAGAAGACCCACCTTGCAGATGTCCT |
| | CACTGGGGCTGGCAGAGCCGGCAACCTGCCTAAGGCTGCT |
| | CAGTCCATTAGGAGCCAGTAGCCTGGAAGATGTCTTTACC |
| | CCCAGCATCAGTTCAAGTGGAGCAGCACATAACTCTTGCC |
| | CTCTGCCTTCCAAGATTCTGGTGCTGAGACTTATGGAGTG |
| | TCTTGGAGGTTGCCTTCTGCCCCCCAACCCTGCTCCCAGC |
| | TGGCCCTCCCAGGCCTGGGTTGCTGGCCTCTGCTTTATCA |
| | GGATTCTCAAGAGGGACAGCTGGTTTATGTTGCATGACTG |
| | TTCCCTGCATATCTGCTCTGGTTTTAAATAGCTTATCTGA |
| | GCAGCTGGAGGACCACATGGGCTTATATGGCGTGGGGTAC |
| | ATGTTCCTGTAGCCTTGTCCCTGGCACCTGCCAAAATAGC |
| | AGCCAACACCCCCCACCCCCACCGCCATCCCCCTGCCCCA |
| | CCCGTCCCCTGTCGCACATTCCTCCCTCCGCAGGGCTGGC |
| | TCACCAGGCCCCAGCCCACATGCCTGCTTAAAGCCCTCTC |
| | CATCCTCTGCCTCACCCAGTCCCCGCTGAGACTGAGCAGA |
| | CGCCTCCAGCGGCCGCCCGCCACCATGGCTGAGAAAGAGT |
| | CAACATCACCACACCTCATGGTTCCCATTCTTCTCCTGGT |
| | TGGATGGATTGTAGGCTGCATCATCGTTATTTACATTGTC |
| | TTCTTCTAAAAGCTTTGGATCCAATCAACCTCTGGATTAC |
| | AAAATTTGTGAAAGATTGACTGGTATTCTTAACTATGTTG |
| | CTCCTTTTACGCTATGTGGATACGCTGCTTTAATGCCTTT |
| | GTATCATGCTATTGCTTCCCGTATGGCTTTCATTTTCTCC |
| | TCCTTGTATAAATCCTGGTTGCTGTCTCTTTATGAGGAGT |
| | TGTGGCCCGTTGTCAGGCAACGTGGCGTGGTGTGCACTGT |
| | GTTTGCTGACGCAACCCCCACTGGTTGGGGCATTGCCACC |
| | ACCTGTCAGCTCCTTTCCGGGACTTTCGCTTTCCCCCTCC |
| | CTATTGCCACGGCGGAACTCATCGCCGCCTGCCTTGCCCG |
| | CTGCTGGACAGGGGCTCGGCTGTTGGGCACTGACAATTCC |
| | GTGGTGTTGTCGGGGAAATCATCGTCCTTTCCTTGGCTGC |
| | TCGCCTGTGTTGCCACCTGGATTCTGCGCGGGACGTCCTT |
| | CTGCTACGTCCCTTCGGCCCTCAATCCAGCGGACCTTCCT |
| | TCCCGCGGCCTGCTGCCGGCTCTGCGGCCTCTTCCGCGTC |
| | TTCGAGATCTGCCTCGACTGTGCCTTCTAGTTGCCAGCCA |
| | TCTGTTGTTTGCCCCTCCCCCGTGCCTTCCTTGACCCTGG |
| | AAGGTGCCACTCCCACTGTCCTTTCCTAATAAAATGAGGA |
| | AATTGCATCGCATTGTCTGAGTAGGTGTCATTCTATTCTG |
| | GGGGGTGGGGTGGGGCAGGACAGCAAGGGGGAGGATTGGG |
| | AAGACAATAGCAGGCATGCTGGGGACTCGAGTTAAGGGCG |
| | AATTCCCGATTAGGATCTTCCTAGAGCATGGCTACGTAGA |
| | TAAGTAGCATGGCGGGTTAATCATTAACTACAAGGAACCC |
| | CTAGTGATGGAGTTGGCCACTCCCTCTCTGCGCGCTCGCT |
| | CGCTCACTGAGGCCGGGCGACCAAAGGTCGCCCGACGCCC |
| | GGGCTTTGCCCGGGCGGCCTCAGTGAGCGAGCGAGCGCGC |
| | AG (SEQ ID NO: 45) |
| pHZ16 | CTGCGCGCTCGCTCGCTCACTGAGGCCGCCCGGGCAAAGC |
| | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGAACTGGCCTGCCCGAGACCAAACGTGCGGAACGTA |
| | GTTAAGTGTTAGAGGTAGGATTTGAAGCCTGTCGATCATT |
| | CTGATTCTCCTTTTCTCTACGTCTGCTTCCTGTCAATGGG |
| | CATCCTCACTGTCAAATGCAGATGGTACAGCAGGGCTTGG |
| | TCTCAGCCAGGCAGGCCTCTCCCCAGTCTCCATGGCTCAG |
| | CTGTCCAGCAGTTTCATCCCTAGACCATCCCAAACATGGT |
| | TGAGAAGCTCTGAGGGGAGGACCCAGCACTGCCCGGCCCC |
| | TGAAGATAATCAGCAGTCCTGCTCAGCATATCAATCCAAG |
| | CCCACTCTAGACAGAGATGCCGGTGCCCAGTTTTCTATTT |
| | TTAACTGGTGTGAACTGAAGGAAAAGCACAGCATTAGAAG |
| | TCCAAGCACCTTCAGATTAAAAATAACTAAGGTAAGGGCC |
| | ATGTGGGTAGGGGAGGTGGTGTGAGACGGTCCTGTCTCTC |
| | CTCTATCTGCCCATCGGCCCTTTGGGGAGGAGGAATGTGC |
| | CCAAGGACTAAAAAAAGGCCCTGGAGCCAGAGGGGCGAGG |
| | GCAGCAGACCTTTCATGGGCAAACCTCAGGGCTGCTGTCG |
| | TCATGGAGAAGACCCACCTTGCAGATGTCCTCACTGGGGC |
| | TGGCAGAGCCGGCAACCTGCCTAAGGCTGCTCAGTCCATT |
| | AGGAGCCAGTAGCCTGGAAGATGTCTTTACCCCCAGCATC |
| | AGTTCAAGTGGAGCAGCACATAACTCTTGCCCTCTGCCTT |
| | CCAAGATTCTGGTGCTGAGACTTATGGAGTGTCTTGGAGG |
| | TTGCCTTCTGCCCCCCAACCCTGCTCCCAGCTGGCCCTCC |
| | CAGGCCTGGGTTGCTGGCCTCTGCTTTATCAGGATTCTCA |
| | AGAGGGACAGCTGGTTTATGTTGCATGACTGTTCCCTGCA |
| | TATCTGCTCTGGTTTTAAATAGCTTATCTGAGCAGCTGGA |
| | GGACCACATGGGCTTATATGGCGTGGGGTACATGTTCCTG |
| | TAGCCTTGTCCCTGGCACCTGCCAAAATAGCAGCCAACAC |
| | CCCCCACCCCCACCGCCATCCCCCTGCCCCACCCGTCCCC |
| | TGTCGCACATTCCTCCCTCCGCAGGGCTGGCTCACCAGGC |
| | CCCAGCCCACATGCCTGCTTAAAGCCCTCTCCATCCTCTG |
| | CCTCACCCAGTCCCCGCTGAGACTGAGCAGACGCCTCCAG |
| | CGGCCGCCCGCCACCATGGCTGAGAAAGAGTCAACATCAC |
| | CACACCTCATGGTTCCCATTCTTCTCCTGGTTGGATGGAT |
| | TGTAGGCTGCATCATCGTTATTTACATTGTCTTCTTCTAA |
| | AAGCTTTGGATCCAATCAACCTCTGGATTACAAAATTTGT |
| | GAAAGATTGACTGGTATTCTTAACTATGTTGCTCCTTTTA |
| | CGCTATGTGGATACGCTGCTTTAATGCCTTTGTATCATGC |
| | TATTGCTTCCCGTATGGCTTTCATTTTCTCCTCCTTGTAT |
| | AAATCCTGGTTGCTGTCTCTTTATGAGGAGTTGTGGCCCG |
| | TTGTCAGGCAACGTGGCGTGGTGTGCACTGTGTTTGCTGA |
| | CGCAACCCCCACTGGTTGGGGCATTGCCACCACCTGTCAG |
| | CTCCTTTCCGGGACTTTCGCTTTCCCCCTCCCTATTGCCA |
| | CGGCGGAACTCATCGCCGCCTGCCTTGCCCGCTGCTGGAC |
| | AGGGGCTCGGCTGTTGGGCACTGACAATTCCGTGGTGTTG |
| | TCGGGGAAATCATCGTCCTTTCCTTGGCTGCTCGCCTGTG |
| | TTGCCACCTGGATTCTGCGCGGGACGTCCTTCTGCTACGT |
| | CCCTTCGGCCCTCAATCCAGCGGACCTTCCTTCCCGCGGC |
| | CTGCTGCCGGCTCTGCGGCCTCTTCCGCGTCTTCGAGATC |
| | TGCCTCGACTGTGCCTTCTAGTTGCCAGCCATCTGTTGTT |
| | TGCCCCTCCCCCGTGCCTTCCTTGACCCTGGAAGGTGCCA |
| | CTCCCACTGTCCTTTCCTAATAAAATGAGGAAATTGCATC |
| | GCATTGTCTGAGTAGGTGTCATTCTATTCTGGGGGGTGGG |
| | GTGGGGCAGGACAGCAAGGGGGAGGATTGGGAAGACAATA |
| | GCAGGCATGCTGGGGACTCGAGTTAAGGGCGAATTCCCGA |
| | TTAGGATCTTCCTAGAGCATGGCTACGTAGATAAGTAGCA |
| | TGGCGGGTTAATCATTAACTACAAGGAACCCCTAGTGATG |
| | GAGTTGGCCACTCCCTCTCTGCGCGCTCGCTCGCTCACTG |
| | AGGCCGGGCGACCAAAGGTCGCCCGACGCCCGGGCTTTGC |
| | CCGGGCGGCCTCAGTGAGCGAGCGAGCGCGCAG |
| | (SEQ ID NO: 46) |
| pHZ17 | CTGCGCGCTCGCTCGCTCACTGAGGCCGCCCGGGCAAAGC |
| | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGCCTTCAGATTAAAAATAACTAAGGTAAGGGCCATG |
| | TGGGTAGGGGAGGTGGTGTGAGACGGTCCTGTCTCTCCTC |
| | TATCTGCCCATCGGCCCTTTGGGGAGGAGGAATGTGCCCA |
| | AGGACTAAAAAAAGGCCCTGGAGCCAGAGGGGCGAGGGCA |
| | GCAGACCTTTCATGGGCAAACCTCAGGGCTGCTGTCGTCA |
| | TGGAGAAGACCCACCTTGCAGATGTCCTCACTGGGGCTGG |
| | CAGAGCCGGCAACCTGCCTAAGGCTGCTCAGTCCATTAGG |
| | AGCCAGTAGCCTGGAAGATGTCTTTACCCCCAGCATCAGT |
| | TCAAGTGGAGCAGCACATAACTCTTGCCCTCTGCCTTCCA |
| | AGATTCTGGTGCTGAGACTTATGGAGTGTCTTGGAGGTTG |
| | CCTTCTGCCCCCCAACCCTGCTCCCAGCTGGCCCTCCCAG |
| | GCCTGGGTTGCTGGCCTCTGCTTTATCAGGATTCTCAAGA |
| | GGGACAGCTGGTTTATGTTGCATGACTGTTCCCTGCATAT |
| | CTGCTCTGGTTTTAAATAGCTTATCTGAGCAGCTGGAGGA |
| | CCACATGGGCTTATATGGCGTGGGGTACATGTTCCTGTAG |
| | CCTTGTCCCTGGCACCTGCCAAAATAGCAGCCAACACCCC |
| | CCACCCCCACCGCCATCCCCCTGCCCCACCCGTCCCCTGT |
| | CGCACATTCCTCCCTCCGCAGGGCTGGCTCACCAGGCCCC |
| | AGCCCACATGCCTGCTTAAAGCCCTCTCCATCCTCTGCCT |
| | CACCCAGTCCCCGCTGAGACTGAGCAGACGCCTCCAGCGG |
| | CCGCCCGCCACCATGGCTGAGAAAGAGTCAACATCACCAC |
| | ACCTCATGGTTCCCATTCTTCTCCTGGTTGGATGGATTGT |
| | AGGCTGCATCATCGTTATTTACATTGTCTTCTTCTAAAAG |
| | CTTTGGATCCAATCAACCTCTGGATTACAAAATTTGTGAA |
| | AGATTGACTGGTATTCTTAACTATGTTGCTCCTTTTACGC |
| | TATGTGGATACGCTGCTTTAATGCCTTTGTATCATGCTAT |
| | TGCTTCCCGTATGGCTTTCATTTTCTCCTCCTTGTATAAA |
| | TCCTGGTTGCTGTCTCTTTATGAGGAGTTGTGGCCCGTTG |
| | TCAGGCAACGTGGCGTGGTGTGCACTGTGTTTGCTGACGC |
| | AACCCCCACTGGTTGGGGCATTGCCACCACCTGTCAGCTC |
| | CTTTCCGGGACTTTCGCTTTCCCCCTCCCTATTGCCACGG |
| | CGGAACTCATCGCCGCCTGCCTTGCCCGCTGCTGGACAGG |
| | GGCTCGGCTGTTGGGCACTGACAATTCCGTGGTGTTGTCG |
| | GGGAAATCATCGTCCTTTCCTTGGCTGCTCGCCTGTGTTG |
| | CCACCTGGATTCTGCGCGGGACGTCCTTCTGCTACGTCCC |
| | TTCGGCCCTCAATCCAGCGGACCTTCCTTCCCGCGGCCTG |
| | CTGCCGGCTCTGCGGCCTCTTCCGCGTCTTCGAGATCTGC |
| | CTCGACTGTGCCTTCTAGTTGCCAGCCATCTGTTGTTTGC |
| | CCCTCCCCCGTGCCTTCCTTGACCCTGGAAGGTGCCACTC |
| | CCACTGTCCTTTCCTAATAAAATGAGGAAATTGCATCGCA |
| | TTGTCTGAGTAGGTGTCATTCTATTCTGGGGGGTGGGGTG |
| | GGGCAGGACAGCAAGGGGGAGGATTGGGAAGACAATAGCA |
| | GGCATGCTGGGGACTCGAGTTAAGGGCGAATTCCCGATTA |
| | GGATCTTCCTAGAGCATGGCTACGTAGATAAGTAGCATGG |
| | CGGGTTAATCATTAACTACAAGGAACCCCTAGTGATGGAG |
| | TTGGCCACTCCCTCTCTGCGCGCTCGCTCGCTCACTGAGG |
| | CCGGGCGACCAAAGGTCGCCCGACGCCCGGGCTTTGCCCG |
| | GGCGGCCTCAGTGAGCGAGCGAGCGCGCAG |
| | (SEQ ID NO: 47) |
| pHZ18 | CTGCGCGCTCGCTCGCTCACTGAGGCCGCCCGGGCAAAGC |
| | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGCCTTCAGATTAAAAATAACTAAGGTAAGGGCCATG |
| | TGGGTAGGGGAGGTGGTGTGAGACGGTCCTGTCTCTCCTC |
| | TATCTGCCCATCGGCCCTTTGGGGAGGAGGAATGTGCCCA |
| | AGGACTAAAAAAAGGCCCTGGAGCCAGAGGGGCGAGGGCA |
| | GCAGACCTTTCATGGGCAAACCTCAGGGCTGCTGTCAACT |
| | GGCCTGCCCGAGACCAAACGTGCGGAACGTAGTTAAGTGT |
| | TAGAGGTAGGATTTGAAGCCTGTCGATCATTCTGATTCTC |
| | CTTTTCTCTACGTCTGCTTCCTGTCAATGGGCATCCTCAC |
| | TGTCAAATGCAGATGGTACAGCAGGGCTTGGTCTCAGCCA |
| | GGCAGGCCTCTCCCCAGTCTCCATGGCTCAGCTGTCCAGC |
| | AGTTTCATCCCTAGACCATCCCAAACATGGTTGAGAAGCT |
| | CTGAGGGGAGGACCCAGCACTGCCCGGCCCCTGAAGATAA |
| | TCAGCAGTCCTGCTCAGCATATCAATCCAAGCCCACTCTA |
| | GACAGAGATGCCGGTGCCCAGTTTTCTATTTTTAACTGGT |
| | GTGAACTGAAGGAAAAGCACAGCATTAGAAGTCCAAGCAG |
| | TCATGGAGAAGACCCACCTTGCAGATGTCCTCACTGGGGC |
| | TGGCAGAGCCGGCAACCTGCCTAAGGCTGCTCAGTCCATT |
| | AGGAGCCAGTAGCCTGGAAGATGTCTTTACCCCCAGCATC |
| | AGTTCAAGTGGAGCAGCACATAACTCTTGCCCTCTGCCTT |
| | CCAAGATTCTGGTGCTGAGACTTATGGAGTGTCTTGGAGG |
| | TTGCCTTCTGCCCCCCAACCCTGCTCCCAGCTGGCCCTCC |
| | CAGGCCTGGGTTGCTGGCCTCTGCTTTATCAGGATTCTCA |
| | AGAGGGACAGCTGGTTTATGTTGCATGACTGTTCCCTGCA |
| | TATCTGCTCTGGTTTTAAATAGCTTATCTGAGCAGCTGGA |
| | GGACCACATGGGCTTATATGGCGTGGGGTACATGTTCCTG |
| | TAGCCTTGTCCCTGGCACCTGCCAAAATAGCAGCCAACAC |
| | CCCCCACCCCCACCGCCATCCCCCTGCCCCACCCGTCCCC |
| | TGTCGCACATTCCTCCCTCCGCAGGGCTGGCTCACCAGGC |
| | CCCAGCCCACATGCCTGCTTAAAGCCCTCTCCATCCTCTG |
| | CCTCACCCAGTCCCCGCTGAGACTGAGCAGACGCCTCCAG |
| | CGGCCGCCCGCCACCATGGCTGAGAAAGAGTCAACATCAC |
| | CACACCTCATGGTTCCCATTCTTCTCCTGGTTGGATGGAT |
| | TGTAGGCTGCATCATCGTTATTTACATTGTCTTCTTCTAA |
| | AAGCTTTGGATCCAATCAACCTCTGGATTACAAAATTTGT |
| | GAAAGATTGACTGGTATTCTTAACTATGTTGCTCCTTTTA |
| | CGCTATGTGGATACGCTGCTTTAATGCCTTTGTATCATGC |
| | TATTGCTTCCCGTATGGCTTTCATTTTCTCCTCCTTGTAT |
| | AAATCCTGGTTGCTGTCTCTTTATGAGGAGTTGTGGCCCG |
| | TTGTCAGGCAACGTGGCGTGGTGTGCACTGTGTTTGCTGA |
| | CGCAACCCCCACTGGTTGGGGCATTGCCACCACCTGTCAG |
| | CTCCTTTCCGGGACTTTCGCTTTCCCCCTCCCTATTGCCA |
| | CGGCGGAACTCATCGCCGCCTGCCTTGCCCGCTGCTGGAC |
| | AGGGGCTCGGCTGTTGGGCACTGACAATTCCGTGGTGTTG |
| | TCGGGGAAATCATCGTCCTTTCCTTGGCTGCTCGCCTGTG |
| | TTGCCACCTGGATTCTGCGCGGGACGTCCTTCTGCTACGT |
| | CCCTTCGGCCCTCAATCCAGCGGACCTTCCTTCCCGCGGC |
| | CTGCTGCCGGCTCTGCGGCCTCTTCCGCGTCTTCGAGATC |
| | TGCCTCGACTGTGCCTTCTAGTTGCCAGCCATCTGTTGTT |
| | TGCCCCTCCCCCGTGCCTTCCTTGACCCTGGAAGGTGCCA |
| | CTCCCACTGTCCTTTCCTAATAAAATGAGGAAATTGCATC |
| | GCATTGTCTGAGTAGGTGTCATTCTATTCTGGGGGGTGGG |
| | GTGGGGCAGGACAGCAAGGGGGAGGATTGGGAAGACAATA |
| | GCAGGCATGCTGGGGACTCGAGTTAAGGGCGAATTCCCGA |
| | TTAGGATCTTCCTAGAGCATGGCTACGTAGATAAGTAGCA |
| | TGGCGGGTTAATCATTAACTACAAGGAACCCCTAGTGATG |
| | GAGTTGGCCACTCCCTCTCTGCGCGCTCGCTCGCTCACTG |
| | AGGCCGGGCGACCAAAGGTCGCCCGACGCCCGGGCTTTGC |
| | CCGGGCGGCCTCAGTGAGCGAGCGAGCGCGCAG |
| | (SEQ ID NO: 48) |
| pHZ19 | CTGCGCGCTCGCTCGCTCACTGAGGCCGCCCGGGCAAAGC |
| distinct sequence elements | CCGGGCGTCGGGCGACCTTTGGTCGCCCGGCCTCAGTGAG |
| are indicated in bold, | CGAGCGAGCGCGCAGAGAGGGAGTGGCCAACTCCATCACT |
| starting with ITR, | AGGGGTTCCTTGTAGTTAATGATTAACCCGCCATGCTACT |
| enhancer/promoter combo | TATCTACGTAGCCATGCTCTAGGAAGATCGGAATTCGCCC |
| (αMHCe, followed by | TTAAGCCTTCAGATTAAAAATAACTAAGGTAAGGGCCATG |
| ACTC1e, followed by | TGGGTAGGGGAGGTGGTGTGAGACGGTCCTGTCTCTCCTC |
| hcTnTp), DWORF (which | TATCTGCCCATCGGCCCTTTGGGGAGGAGGAATGTGCCCA |
| is also underlined), WPRE, | AGGACTAAAAAAAGGCCCTGGAGCCAGAGGGGCGAGGGCA |
| polyA (hGHpA), ITR | GCAGACCTTTCATGGGCAAACCTCAGGGCTGCTGTCAACT |
| | GGCCTGCCCGAGACCAAACGTGCGGAACGTAGTTAAGTGT |
| | TAGAGGTAGGATTTGAAGCCTGTCGATCATTCTGATTCTC |
| | CTTTTCTCTACGTCTGCTTCCTGTCAATGGGCATCCTCAC |
| | TGTCAAATGCAGATGGTACAGCAGGGCTTGGTCTCAGCCA |
| | GGCAGGCCTCTCCCCAGTCTCCATGGCTCAGCTGTCCAGC |
| | AGTTTCATCCCTAGACCATCCCAAACATGGTTGAGAAGCT |
| | CTGAGGGGAGGACCCAGCACTGCCCGGCCCCTGAAGATAA |
| | TCAGCAGTCCTGCTCAGCATATCAATCCAAGCCCACTCTA |
| | GACAGAGATGCCGGTGCCCAGTTTTCTATTTTTAACTGGT |
| | GTGAACTGAAGGAAAAGCACAGCATTAGAAGTCCAAGCAG |
| | TCATGGAGAAGACCCACCTTGCAGATGTCCTCACTGGGGC |
| | TGGCAGAGCCGGCAACCTGCCTAAGGCTGCTCAGTCCATT |
| | AGGAGCCAGTAGCCTGGAAGATGTCTTTACCCCCAGCATC |
| | AGTTCAAGTGGAGCAGCACATAACTCTTGCCCTCTGCCTT |
| | CCAAGATTCTGGTGCTGAGACTTATGGAGTGTCTTGGAGG |
| | TTGCCTTCTGCCCCCCAACCCTGCTCCCAGCTGGCCCTCC |
| | CAGGCCTGGGTTGCTGGCCTCTGCTTTATCAGGATTCTCA |
| | AGAGGGACAGCTGGTTTATGTTGCATGACTGTTCCCTGCA |
| | TATCTGCTCTGGTTTTAAATAGCTTATCTGAGCAGCTGGA |
| | GGACCACATGGGCTTATATGGCGTGGGGTACATGTTCCTG |
| | TAGCCTTGTCCCTGGCACCTGCCAAAATAGCAGCCAACAC |
| | CCCCCACCCCCACCGCCATCCCCCTGCCCCACCCGTCCCC |
| | TGTCGCACATTCCTCCCTCCGCAGGGCTGGCTCACCAGGC |
| | CCCAGCCCACATGCCTGCTTAAAGCCCTCTCCATCCTCTG |
| | CCTCACCCAGTCCCCGCTGAGACTGAGCAGACGCCTCCAT |
| | AACTGGTAAGTACCGCCTATAGACTCTATAGGCACACCCC |
| | TTTGGCTCTTATGCATGCTGACAGACTAACAGACTGTTCC |
| | TTTCCTGGGTCTTTTCTGCAGGCCTGTACGGAAGTGTTAC |
| | TTCTGCTCTAAAAGCTGCGGAATTGTACCCGCGGCCGATC |
| | CAATCGATACAGATCTAGCGGCCGCCCGCCACCATGGCTG |
| | AGAAAGAGTCAACATCACCACACCTCATGGTTCCCATTCT |
| | TCTCCTGGTTGGATGGATTGTAGGCTGCATCATCGTTATT |
| | TACATTGTCTTCTTCTAAAAGCTTTGGATCCAATCAACCT |
| | CTGGATTACAAAATTTGTGAAAGATTGACTGGTATTCTTA |
| | ACTATGTTGCTCCTTTTACGCTATGTGGATACGCTGCTTT |
| | AATGCCTTTGTATCATGCTATTGCTTCCCGTATGGCTTTC |
| | ATTTTCTCCTCCTTGTATAAATCCTGGTTGCTGTCTCTTT |
| | ATGAGGAGTTGTGGCCCGTTGTCAGGCAACGTGGCGTGGT |
| | GTGCACTGTGTTTGCTGACGCAACCCCCACTGGTTGGGGC |
| | ATTGCCACCACCTGTCAGCTCCTTTCCGGGACTTTCGCTT |
| | TCCCCCTCCCTATTGCCACGGCGGAACTCATCGCCGCCTG |
| | CCTTGCCCGCTGCTGGACAGGGGCTCGGCTGTTGGGCACT |
| | GACAATTCCGTGGTGTTGTCGGGGAAATCATCGTCCTTTC |
| | CTTGGCTGCTCGCCTGTGTTGCCACCTGGATTCTGCGCGG |
| | GACGTCCTTCTGCTACGTCCCTTCGGCCCTCAATCCAGCG |
| | GACCTTCCTTCCCGCGGCCTGCTGCCGGCTCTGCGGCCTC |
| | TTCCGCGTCTTCGAGATCTGCCTCGACTGTGCCTTCTAGT |
| | TGCCAGCCATCTGTTGTTTGCCCCTCCCCCGTGCCTTCCT |
| | TGACCCTGGAAGGTGCCACTCCCACTGTCCTTTCCTAATA |
| | AAATGAGGAAATTGCATCGCATTGTCTGAGTAGGTGTCAT |
| | TCTATTCTGGGGGGTGGGGTGGGGCAGGACAGCAAGGGGG |
| | AGGATTGGGAAGACAATAGCAGGCATGCTGGGGACTCGAG |
| | TTAAGGGCGAATTCCCGATTAGGATCTTCCTAGAGCATGG |
| | CTACGTAGATAAGTAGCATGGCGGGTTAATCATTAACTAC |
| | AAGGAACCCCTAGTGATGGAGTTGGCCACTCCCTCTCTGC |
| | GCGCTCGCTCGCTCACTGAGGCCGGGCGACCAAAGGTCGC |
| | CCGACGCCCGGGCTTTGCCCGGGCGGCCTCAGTGAGCGAG |
| | CGAGCGCGCAG (SEQ ID NO: 49) |
| pHZ20 | CTGCGCGCTCGCTCGCTCACTGAGGCCGCCCGGGCAAAGC |
| | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGGTCATGGAGAAGACCCACCTTGCAGATGTCCTCAC |
| | TGGGGCTGGCAGAGCCGGCAACCTGCCTAAGGCTGCTCAG |
| | TCCATTAGGAGCCAGTAGCCTGGAAGATGTCTTTACCCCC |
| | AGCATCAGTTCAAGTGGAGCAGCACATAACTCTTGCCCTC |
| | TGCCTTCCAAGATTCTGGTGCTGAGACTTATGGAGTGTCT |
| | TGGAGGTTGCCTTCTGCCCCCCAACCCTGCTCCCAGCTGG |
| | CCCTCCCAGGCCTGGGTTGCTGGCCTCTGCTTTATCAGGA |
| | TTCTCAAGAGGGACAGCTGGTTTATGTTGCATGACTGTTC |
| | CCTGCATATCTGCTCTGGTTTTAAATAGCTTATCTGAGCA |
| | GCTGGAGGACCACATGGGCTTATATGGCGTGGGGTACATG |
| | TTCCTGTAGCCTTGTCCCTGGCACCTGCCAAAATAGCAGC |
| | CAACACCCCCCACCCCCACCGCCATCCCCCTGCCCCACCC |
| | GTCCCCTGTCGCACATTCCTCCCTCCGCAGGGCTGGCTCA |
| | CCAGGCCCCAGCCCACATGCCTGCTTAAAGCCCTCTCCAT |
| | CCTCTGCCTCACCCAGTCCCCGCTGAGACTGAGCAGACGC |
| | CTCCATAACTGGTAAGTACCGCCTATAGACTCTATAGGCA |
| | CACCCCTTTGGCTCTTATGCATGCTGACAGACTAACAGAC |
| | TGTTCCTTTCCTGGGTCTTTTCTGCAGGCCTGTACGGAAG |
| | TGTTACTTCTGCTCTAAAAGCTGCGGAATTGTACCCGCGG |
| | CCGATCCAATCGATACAGATCTAGCGGCCGCCCGCCACCA |
| | TGGCTGAGAAAGAGTCAACATCACCACACCTCATGGTTCC |
| | CATTCTTCTCCTGGTTGGATGGATTGTAGGCTGCATCATC |
| | GTTATTTACATTGTCTTCTTCTAAAAGCTTTGGATCCAAT |
| | CAACCTCTGGATTACAAAATTTGTGAAAGATTGACTGGTA |
| | TTCTTAACTATGTTGCTCCTTTTACGCTATGTGGATACGC |
| | TGCTTTAATGCCTTTGTATCATGCTATTGCTTCCCGTATG |
| | GCTTTCATTTTCTCCTCCTTGTATAAATCCTGGTTGCTGT |
| | CTCTTTATGAGGAGTTGTGGCCCGTTGTCAGGCAACGTGG |
| | CGTGGTGTGCACTGTGTTTGCTGACGCAACCCCCACTGGT |
| | TGGGGCATTGCCACCACCTGTCAGCTCCTTTCCGGGACTT |
| | TCGCTTTCCCCCTCCCTATTGCCACGGCGGAACTCATCGC |
| | CGCCTGCCTTGCCCGCTGCTGGACAGGGGCTCGGCTGTTG |
| | GGCACTGACAATTCCGTGGTGTTGTCGGGGAAATCATCGT |
| | CCTTTCCTTGGCTGCTCGCCTGTGTTGCCACCTGGATTCT |
| | GCGCGGGACGTCCTTCTGCTACGTCCCTTCGGCCCTCAAT |
| | CCAGCGGACCTTCCTTCCCGCGGCCTGCTGCCGGCTCTGC |
| | GGCCTCTTCCGCGTCTTCGAGATCTGCCTCGACTGTGCCT |
| | TCTAGTTGCCAGCCATCTGTTGTTTGCCCCTCCCCCGTGC |
| | CTTCCTTGACCCTGGAAGGTGCCACTCCCACTGTCCTTTC |
| | CTAATAAAATGAGGAAATTGCATCGCATTGTCTGAGTAGG |
| | TGTCATTCTATTCTGGGGGGTGGGGTGGGGCAGGACAGCA |
| | AGGGGGAGGATTGGGAAGACAATAGCAGGCATGCTGGGGA |
| | CTCGAGTTAAGGGCGAATTCCCGATTAGGATCTTCCTAGA |
| | GCATGGCTACGTAGATAAGTAGCATGGCGGGTTAATCATT |
| | AACTACAAGGAACCCCTAGTGATGGAGTTGGCCACTCCCT |
| | CTCTGCGCGCTCGCTCGCTCACTGAGGCCGGGCGACCAAA |
| | GGTCGCCCGACGCCCGGGCTTTGCCCGGGCGGCCTCAGTG |
| | AGCGAGCGAGCGCGCAG (SEQ ID NO: 50) |
| pHZ21 | CTGCGCGCTCGCTCGCTCACTGAGGCCGCCCGGGCAAAGC |
| distinct sequence elements | CCGGGCGTCGGGCGACCTTTGGTCGCCCGGCCTCAGTGAG |
| are indicated in bold, | CGAGCGAGCGCGCAGAGAGGGAGTGGCCAACTCCATCACT |
| starting with ITR, | AGGGGTTCCTTGTAGTTAATGATTAACCCGCCATGCTACT |
| enhancer/promoter combo | TATCTACGTAGCCATGCTCTAGGAAGATCGGAATTCGCCC |
| (ACTC1e, followed by | TTAAGAACTGGCCTGCCCGAGACCAAACGTGCGGAACGTA |
| αMHCe, followed by | GTTAAGTGTTAGAGGTAGGATTTGAAGCCTGTCGATCATT |
| hcTnTp), DWORF (which | CTGATTCTCCTTTTCTCTACGTCTGCTTCCTGTCAATGGG |
| is also underlined), WPRE, | CATCCTCACTGTCAAATGCAGATGGTACAGCAGGGCTTGG |
| polyA (hGHpA), ITR | TCTCAGCCAGGCAGGCCTCTCCCCAGTCTCCATGGCTCAG |
| | CTGTCCAGCAGTTTCATCCCTAGACCATCCCAAACATGGT |
| | TGAGAAGCTCTGAGGGGAGGACCCAGCACTGCCCGGCCCC |
| | TGAAGATAATCAGCAGTCCTGCTCAGCATATCAATCCAAG |
| | CCCACTCTAGACAGAGATGCCGGTGCCCAGTTTTCTATTT |
| | TTAACTGGTGTGAACTGAAGGAAAAGCACAGCATTAGAAG |
| | TCCAAGCACCTTCAGATTAAAAATAACTAAGGTAAGGGCC |
| | ATGTGGGTAGGGGAGGTGGTGTGAGACGGTCCTGTCTCTC |
| | CTCTATCTGCCCATCGGCCCTTTGGGGAGGAGGAATGTGC |
| | CCAAGGACTAAAAAAAGGCCCTGGAGCCAGAGGGGCGAGG |
| | GCAGCAGACCTTTCATGGGCAAACCTCAGGGCTGCTGTCG |
| | TCATGGAGAAGACCCACCTTGCAGATGTCCTCACTGGGGC |
| | TGGCAGAGCCGGCAACCTGCCTAAGGCTGCTCAGTCCATT |
| | AGGAGCCAGTAGCCTGGAAGATGTCTTTACCCCCAGCATC |
| | AGTTCAAGTGGAGCAGCACATAACTCTTGCCCTCTGCCTT |
| | CCAAGATTCTGGTGCTGAGACTTATGGAGTGTCTTGGAGG |
| | TTGCCTTCTGCCCCCCAACCCTGCTCCCAGCTGGCCCTCC |
| | CAGGCCTGGGTTGCTGGCCTCTGCTTTATCAGGATTCTCA |
| | AGAGGGACAGCTGGTTTATGTTGCATGACTGTTCCCTGCA |
| | TATCTGCTCTGGTTTTAAATAGCTTATCTGAGCAGCTGGA |
| | GGACCACATGGGCTTATATGGCGTGGGGTACATGTTCCTG |
| | TAGCCTTGTCCCTGGCACCTGCCAAAATAGCAGCCAACAC |
| | CCCCCACCCCCACCGCCATCCCCCTGCCCCACCCGTCCCC |
| | TGTCGCACATTCCTCCCTCCGCAGGGCTGGCTCACCAGGC |
| | CCCAGCCCACATGCCTGCTTAAAGCCCTCTCCATCCTCTG |
| | CCTCACCCAGTCCCCGCTGAGACTGAGCAGACGCCTCCAT |
| | AACTGGTAAGTACCGCCTATAGACTCTATAGGCACACCCC |
| | TTTGGCTCTTATGCATGCTGACAGACTAACAGACTGTTCC |
| | TTTCCTGGGTCTTTTCTGCAGGCCTGTACGGAAGTGTTAC |
| | TTCTGCTCTAAAAGCTGCGGAATTGTACCCGCGGCCGATC |
| | CAATCGATACAGATCTAGCGGCCGCCCGCCACCATGGCTG |
| | AGAAAGAGTCAACATCACCACACCTCATGGTTCCCATTCT |
| | TCTCCTGGTTGGATGGATTGTAGGCTGCATCATCGTTATT |
| | TACATTGTCTTCTTCTAAAAGCTTTGGATCCAATCAACCT |
| | CTGGATTACAAAATTTGTGAAAGATTGACTGGTATTCTTA |
| | ACTATGTTGCTCCTTTTACGCTATGTGGATACGCTGCTTT |
| | AATGCCTTTGTATCATGCTATTGCTTCCCGTATGGCTTTC |
| | ATTTTCTCCTCCTTGTATAAATCCTGGTTGCTGTCTCTTT |
| | ATGAGGAGTTGTGGCCCGTTGTCAGGCAACGTGGCGTGGT |
| | GTGCACTGTGTTTGCTGACGCAACCCCCACTGGTTGGGGC |
| | ATTGCCACCACCTGTCAGCTCCTTTCCGGGACTTTCGCTT |
| | TCCCCCTCCCTATTGCCACGGCGGAACTCATCGCCGCCTG |
| | CCTTGCCCGCTGCTGGACAGGGGCTCGGCTGTTGGGCACT |
| | GACAATTCCGTGGTGTTGTCGGGGAAATCATCGTCCTTTC |
| | CTTGGCTGCTCGCCTGTGTTGCCACCTGGATTCTGCGCGG |
| | GACGTCCTTCTGCTACGTCCCTTCGGCCCTCAATCCAGCG |
| | GACCTTCCTTCCCGCGGCCTGCTGCCGGCTCTGCGGCCTC |
| | TTCCGCGTCTTCGAGATCTGCCTCGACTGTGCCTTCTAGT |
| | TGCCAGCCATCTGTTGTTTGCCCCTCCCCCGTGCCTTCCT |
| | TGACCCTGGAAGGTGCCACTCCCACTGTCCTTTCCTAATA |
| | AAATGAGGAAATTGCATCGCATTGTCTGAGTAGGTGTCAT |
| | TCTATTCTGGGGGGTGGGGTGGGGCAGGACAGCAAGGGGG |
| | AGGATTGGGAAGACAATAGCAGGCATGCTGGGGACTCGAG |
| | TTAAGGGCGAATTCCCGATTAGGATCTTCCTAGAGCATGG |
| | CTACGTAGATAAGTAGCATGGCGGGTTAATCATTAACTAC |
| | AAGGAACCCCTAGTGATGGAGTTGGCCACTCCCTCTCTGC |
| | GCGCTCGCTCGCTCACTGAGGCCGGGCGACCAAAGGTCGC |
| | CCGACGCCCGGGCTTTGCCCGGGCGGCCTCAGTGAGCGAG |
| | CGAGCGCGCAG (SEQ ID NO: 51) |
| pHZ22 | CTGCGCGCTCGCTCGCTCACTGAGGCCGCCCGGGCAAAGC |
| | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGAACTGGCCTGCCCGAGACCAAACGTGCGGAACGTA |
| | GTTAAGTGTTAGAGGTAGGATTTGAAGCCTGTCGATCATT |
| | CTGATTCTCCTTTTCTCTACGTCTGCTTCCTGTCAATGGG |
| | CATCCTCACTGTCAAATGCAGATGGTACAGCAGGGCTTGG |
| | TCTCAGCCAGGCAGGCCTCTCCCCAGTCTCCATGGCTCAG |
| | CTGTCCAGCAGTTTCATCCCTAGACCATCCCAAACATGGT |
| | TGAGAAGCTCTGAGGGGAGGACCCAGCACTGCCCGGCCCC |
| | TGAAGATAATCAGCAGTCCTGCTCAGCATATCAATCCAAG |
| | CCCACTCTAGACAGAGATGCCGGTGCCCAGTTTTCTATTT |
| | TTAACTGGTGTGAACTGAAGGAAAAGCACAGCATTAGAAG |
| | TCCAAGCAGTCATGGAGAAGACCCACCTTGCAGATGTCCT |
| | CACTGGGGCTGGCAGAGCCGGCAACCTGCCTAAGGCTGCT |
| | CAGTCCATTAGGAGCCAGTAGCCTGGAAGATGTCTTTACC |
| | CCCAGCATCAGTTCAAGTGGAGCAGCACATAACTCTTGCC |
| | CTCTGCCTTCCAAGATTCTGGTGCTGAGACTTATGGAGTG |
| | TCTTGGAGGTTGCCTTCTGCCCCCCAACCCTGCTCCCAGC |
| | TGGCCCTCCCAGGCCTGGGTTGCTGGCCTCTGCTTTATCA |
| | GGATTCTCAAGAGGGACAGCTGGTTTATGTTGCATGACTG |
| | TTCCCTGCATATCTGCTCTGGTTTTAAATAGCTTATCTGA |
| | GCAGCTGGAGGACCACATGGGCTTATATGGCGTGGGGTAC |
| | ATGTTCCTGTAGCCTTGTCCCTGGCACCTGCCAAAATAGC |
| | AGCCAACACCCCCCACCCCCACCGCCATCCCCCTGCCCCA |
| | CCCGTCCCCTGTCGCACATTCCTCCCTCCGCAGGGCTGGC |
| | TCACCAGGCCCCAGCCCACATGCCTGCTTAAAGCCCTCTC |
| | CATCCTCTGCCTCACCCAGTCCCCGCTGAGACTGAGCAGA |
| | CGCCTCCATAACTGGTAAGTACCGCCTATAGACTCTATAG |
| | GCACACCCCTTTGGCTCTTATGCATGCTGACAGACTAACA |
| | GACTGTTCCTTTCCTGGGTCTTTTCTGCAGGCCTGTACGG |
| | AAGTGTTACTTCTGCTCTAAAAGCTGCGGAATTGTACCCG |
| | CGGCCGATCCAATCGATACAGATCTAGCGGCCGCCCGCCA |
| | CCATGGCTGAGAAAGAGTCAACATCACCACACCTCATGGT |
| | TCCCATTCTTCTCCTGGTTGGATGGATTGTAGGCTGCATC |
| | ATCGTTATTTACATTGTCTTCTTCTAAAAGCTTTGGATCC |
| | AATCAACCTCTGGATTACAAAATTTGTGAAAGATTGACTG |
| | GTATTCTTAACTATGTTGCTCCTTTTACGCTATGTGGATA |
| | CGCTGCTTTAATGCCTTTGTATCATGCTATTGCTTCCCGT |
| | ATGGCTTTCATTTTCTCCTCCTTGTATAAATCCTGGTTGC |
| | TGTCTCTTTATGAGGAGTTGTGGCCCGTTGTCAGGCAACG |
| | TGGCGTGGTGTGCACTGTGTTTGCTGACGCAACCCCCACT |
| | GGTTGGGGCATTGCCACCACCTGTCAGCTCCTTTCCGGGA |
| | CTTTCGCTTTCCCCCTCCCTATTGCCACGGCGGAACTCAT |
| | CGCCGCCTGCCTTGCCCGCTGCTGGACAGGGGCTCGGCTG |
| | TTGGGCACTGACAATTCCGTGGTGTTGTCGGGGAAATCAT |
| | CGTCCTTTCCTTGGCTGCTCGCCTGTGTTGCCACCTGGAT |
| | TCTGCGCGGGACGTCCTTCTGCTACGTCCCTTCGGCCCTC |
| | AATCCAGCGGACCTTCCTTCCCGCGGCCTGCTGCCGGCTC |
| | TGCGGCCTCTTCCGCGTCTTCGAGATCTGCCTCGACTGTG |
| | CCTTCTAGTTGCCAGCCATCTGTTGTTTGCCCCTCCCCCG |
| | TGCCTTCCTTGACCCTGGAAGGTGCCACTCCCACTGTCCT |
| | TTCCTAATAAAATGAGGAAATTGCATCGCATTGTCTGAGT |
| | AGGTGTCATTCTATTCTGGGGGGTGGGGTGGGGCAGGACA |
| | GCAAGGGGGAGGATTGGGAAGACAATAGCAGGCATGCTGG |
| | GGACTCGAGTTAAGGGCGAATTCCCGATTAGGATCTTCCT |
| | AGAGCATGGCTACGTAGATAAGTAGCATGGCGGGTTAATC |
| | ATTAACTACAAGGAACCCCTAGTGATGGAGTTGGCCACTC |
| | CCTCTCTGCGCGCTCGCTCGCTCACTGAGGCCGGGCGACC |
| | AAAGGTCGCCCGACGCCCGGGCTTTGCCCGGGCGGCCTCA |
| | GTGAGCGAGCGAGCGCGCAG (SEQ ID NO: 52) |
| pHZ23 | CTGCGCGCTCGCTCGCTCACTGAGGCCGCCCGGGCAAAGC |
| | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGCCTTCAGATTAAAAATAACTAAGGTAAGGGCCATG |
| | TGGGTAGGGGAGGTGGTGTGAGACGGTCCTGTCTCTCCTC |
| | TATCTGCCCATCGGCCCTTTGGGGAGGAGGAATGTGCCCA |
| | AGGACTAAAAAAAGGCCCTGGAGCCAGAGGGGCGAGGGCA |
| | GCAGACCTTTCATGGGCAAACCTCAGGGCTGCTGTCGTCA |
| | TGGAGAAGACCCACCTTGCAGATGTCCTCACTGGGGCTGG |
| | CAGAGCCGGCAACCTGCCTAAGGCTGCTCAGTCCATTAGG |
| | AGCCAGTAGCCTGGAAGATGTCTTTACCCCCAGCATCAGT |
| | TCAAGTGGAGCAGCACATAACTCTTGCCCTCTGCCTTCCA |
| | AGATTCTGGTGCTGAGACTTATGGAGTGTCTTGGAGGTTG |
| | CCTTCTGCCCCCCAACCCTGCTCCCAGCTGGCCCTCCCAG |
| | GCCTGGGTTGCTGGCCTCTGCTTTATCAGGATTCTCAAGA |
| | GGGACAGCTGGTTTATGTTGCATGACTGTTCCCTGCATAT |
| | CTGCTCTGGTTTTAAATAGCTTATCTGAGCAGCTGGAGGA |
| | CCACATGGGCTTATATGGCGTGGGGTACATGTTCCTGTAG |
| | CCTTGTCCCTGGCACCTGCCAAAATAGCAGCCAACACCCC |
| | CCACCCCCACCGCCATCCCCCTGCCCCACCCGTCCCCTGT |
| | CGCACATTCCTCCCTCCGCAGGGCTGGCTCACCAGGCCCC |
| | AGCCCACATGCCTGCTTAAAGCCCTCTCCATCCTCTGCCT |
| | CACCCAGTCCCCGCTGAGACTGAGCAGACGCCTCCATAAC |
| | TGGTAAGTACCGCCTATAGACTCTATAGGCACACCCCTTT |
| | GGCTCTTATGCATGCTGACAGACTAACAGACTGTTCCTTT |
| | CCTGGGTCTTTTCTGCAGGCCTGTACGGAAGTGTTACTTC |
| | TGCTCTAAAAGCTGCGGAATTGTACCCGCGGCCGATCCAA |
| | TCGATACAGATCTAGCGGCCGCCCGCCACCATGGCTGAGA |
| | AAGAGTCAACATCACCACACCTCATGGTTCCCATTCTTCT |
| | CCTGGTTGGATGGATTGTAGGCTGCATCATCGTTATTTAC |
| | ATTGTCTTCTTCTAAAAGCTTTGGATCCAATCAACCTCTG |
| | GATTACAAAATTTGTGAAAGATTGACTGGTATTCTTAACT |
| | ATGTTGCTCCTTTTACGCTATGTGGATACGCTGCTTTAAT |
| | GCCTTTGTATCATGCTATTGCTTCCCGTATGGCTTTCATT |
| | TTCTCCTCCTTGTATAAATCCTGGTTGCTGTCTCTTTATG |
| | AGGAGTTGTGGCCCGTTGTCAGGCAACGTGGCGTGGTGTG |
| | CACTGTGTTTGCTGACGCAACCCCCACTGGTTGGGGCATT |
| | GCCACCACCTGTCAGCTCCTTTCCGGGACTTTCGCTTTCC |
| | CCCTCCCTATTGCCACGGCGGAACTCATCGCCGCCTGCCT |
| | TGCCCGCTGCTGGACAGGGGCTCGGCTGTTGGGCACTGAC |
| | AATTCCGTGGTGTTGTCGGGGAAATCATCGTCCTTTCCTT |
| | GGCTGCTCGCCTGTGTTGCCACCTGGATTCTGCGCGGGAC |
| | GTCCTTCTGCTACGTCCCTTCGGCCCTCAATCCAGCGGAC |
| | CTTCCTTCCCGCGGCCTGCTGCCGGCTCTGCGGCCTCTTC |
| | CGCGTCTTCGAGATCTGCCTCGACTGTGCCTTCTAGTTGC |
| | CAGCCATCTGTTGTTTGCCCCTCCCCCGTGCCTTCCTTGA |
| | CCCTGGAAGGTGCCACTCCCACTGTCCTTTCCTAATAAAA |
| | TGAGGAAATTGCATCGCATTGTCTGAGTAGGTGTCATTCT |
| | ATTCTGGGGGGTGGGGTGGGGCAGGACAGCAAGGGGGAGG |
| | ATTGGGAAGACAATAGCAGGCATGCTGGGGACTCGAGTTA |
| | AGGGCGAATTCCCGATTAGGATCTTCCTAGAGCATGGCTA |
| | CGTAGATAAGTAGCATGGCGGGTTAATCATTAACTACAAG |
| | GAACCCCTAGTGATGGAGTTGGCCACTCCCTCTCTGCGCG |
| | CTCGCTCGCTCACTGAGGCCGGGCGACCAAAGGTCGCCCG |
| | ACGCCCGGGCTTTGCCCGGGCGGCCTCAGTGAGCGAGCGA |
| | GCGCGCAG (SEQ ID NO: 53) |
| pHZ24 | CTGCGCGCTCGCTCGCTCACTGAGGCCGCCCGGGCAAAGC |
| | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGGTCATGGAGAAGACCCACCTTGCAGATGTCCTCAC |
| | TGGGGCTGGCAGAGCCGGCAACCTGCCTAAGGCTGCTCAG |
| | TCCATTAGGAGCCAGTAGCCTGGAAGATGTCTTTACCCCC |
| | AGCATCAGTTCAAGTGGAGCAGCACATAACTCTTGCCCTC |
| | TGCCTTCCAAGATTCTGGTGCTGAGACTTATGGAGTGTCT |
| | TGGAGGTTGCCTTCTGCCCCCCAACCCTGCTCCCAGCTGG |
| | CCCTCCCAGGCCTGGGTTGCTGGCCTCTGCTTTATCAGGA |
| | TTCTCAAGAGGGACAGCTGGTTTATGTTGCATGACTGTTC |
| | CCTGCATATCTGCTCTGGTTTTAAATAGCTTATCTGAGCA |
| | GCTGGAGGACCACATGGGCTTATATGGCGTGGGGTACATG |
| | TTCCTGTAGCCTTGTCCCTGGCACCTGCCAAAATAGCAGC |
| | CAACACCCCCCACCCCCACCGCCATCCCCCTGCCCCACCC |
| | GTCCCCTGTCGCACATTCCTCCCTCCGCAGGGCTGGCTCA |
| | CCAGGCCCCAGCCCACATGCCTGCTTAAAGCCCTCTCCAT |
| | CCTCTGCCTCACCCAGTCCCCGCTGAGACTGAGCAGACGC |
| | CTCCAGCGGCCGCCCGCCACCATGGCCGAGAAGGAATCTA |
| | CCAGCCCCCACCTGATGGTGCCTATTCTGCTGCTGGTGGG |
| | CTGGATCGTCGGCTGCATCATCGTGATCTACATCGTGTTC |
| | TTCTGAAAGCTTTGGATCCAATCAACCTCTGGATTACAAA |
| | ATTTGTGAAAGATTGACTGGTATTCTTAACTATGTTGCTC |
| | CTTTTACGCTATGTGGATACGCTGCTTTAATGCCTTTGTA |
| | TCATGCTATTGCTTCCCGTATGGCTTTCATTTTCTCCTCC |
| | TTGTATAAATCCTGGTTGCTGTCTCTTTATGAGGAGTTGT |
| | GGCCCGTTGTCAGGCAACGTGGCGTGGTGTGCACTGTGTT |
| | TGCTGACGCAACCCCCACTGGTTGGGGCATTGCCACCACC |
| | TGTCAGCTCCTTTCCGGGACTTTCGCTTTCCCCCTCCCTA |
| | TTGCCACGGCGGAACTCATCGCCGCCTGCCTTGCCCGCTG |
| | CTGGACAGGGGCTCGGCTGTTGGGCACTGACAATTCCGTG |
| | GTGTTGTCGGGGAAATCATCGTCCTTTCCTTGGCTGCTCG |
| | CCTGTGTTGCCACCTGGATTCTGCGCGGGACGTCCTTCTG |
| | CTACGTCCCTTCGGCCCTCAATCCAGCGGACCTTCCTTCC |
| | CGCGGCCTGCTGCCGGCTCTGCGGCCTCTTCCGCGTCTTC |
| | GAGATCTGCCTCGACTGTGCCTTCTAGTTGCCAGCCATCT |
| | GTTGTTTGCCCCTCCCCCGTGCCTTCCTTGACCCTGGAAG |
| | GTGCCACTCCCACTGTCCTTTCCTAATAAAATGAGGAAAT |
| | TGCATCGCATTGTCTGAGTAGGTGTCATTCTATTCTGGGG |
| | GGTGGGGTGGGGCAGGACAGCAAGGGGGAGGATTGGGAAG |
| | ACAATAGCAGGCATGCTGGGGACTCGAGTTAAGGGCGAAT |
| | TCCCGATTAGGATCTTCCTAGAGCATGGCTACGTAGATAA |
| | GTAGCATGGCGGGTTAATCATTAACTACAAGGAACCCCTA |
| | GTGATGGAGTTGGCCACTCCCTCTCTGCGCGCTCGCTCGC |
| | TCACTGAGGCCGGGCGACCAAAGGTCGCCCGACGCCCGGG |
| | CTTTGCCCGGGCGGCCTCAGTGAGCGAGCGAGCGCGCAG |
| | (SEQ ID NO: 54) |
| pHZ25 | CTGCGCGCTCGCTCGCTCACTGAGGCCGCCCGGGCAAAGC |
| distinct sequence elements | CCGGGCGTCGGGCGACCTTTGGTCGCCCGGCCTCAGTGAG |
| are indicated in bold, | CGAGCGAGCGCGCAGAGAGGGAGTGGCCAACTCCATCACT |
| starting with ITR, | AGGGGTTCCTTGTAGTTAATGATTAACCCGCCATGCTACT |
| promoter/enhancer combo | TATCTACGTAGCCATGCTCTAGGAAGATCGGAATTCGCCC |
| (αMHCe, followed by | TTAAGCCTTCAGATTAAAAATAACTAAGGTAAGGGCCATG |
| hcTnTp), DWORF (which | TGGGTAGGGGAGGTGGTGTGAGACGGTCCTGTCTCTCCTC |
| is also underlined), WPRE, | TATCTGCCCATCGGCCCTTTGGGGAGGAGGAATGTGCCCA |
| polyA (hGHpA), SV40pA, | AGGACTAAAAAAAGGCCCTGGAGCCAGAGGGGCGAGGGCA |
| optimized DWORF (which | GCAGACCTTTCATGGGCAAACCTCAGGGCTGCTGTCGTCA |
| is also underlined), | TGGAGAAGACCCACCTTGCAGATGTCCTCACTGGGGCTGG |
| promoter/enhancer combo | CAGAGCCGGCAACCTGCCTAAGGCTGCTCAGTCCATTAGG |
| (ccTnTp, followed by | AGCCAGTAGCCTGGAAGATGTCTTTACCCCCAGCATCAGT |
| ACTC1e), ITR | TCAAGTGGAGCAGCACATAACTCTTGCCCTCTGCCTTCCA |
| | AGATTCTGGTGCTGAGACTTATGGAGTGTCTTGGAGGTTG |
| | CCTTCTGCCCCCCAACCCTGCTCCCAGCTGGCCCTCCCAG |
| | GCCTGGGTTGCTGGCCTCTGCTTTATCAGGATTCTCAAGA |
| | GGGACAGCTGGTTTATGTTGCATGACTGTTCCCTGCATAT |
| | CTGCTCTGGTTTTAAATAGCTTATCTGAGCAGCTGGAGGA |
| | CCACATGGGCTTATATGGCGTGGGGTACATGTTCCTGTAG |
| | CCTTGTCCCTGGCACCTGCCAAAATAGCAGCCAACACCCC |
| | CCACCCCCACCGCCATCCCCCTGCCCCACCCGTCCCCTGT |
| | CGCACATTCCTCCCTCCGCAGGGCTGGCTCACCAGGCCCC |
| | AGCCCACATGCCTGCTTAAAGCCCTCTCCATCCTCTGCCT |
| | CACCCAGTCCCCGCTGAGACTGAGCAGACGCCTCCATAAC |
| | TGGTAAGTACCGCCTATAGACTCTATAGGCACACCCCTTT |
| | GGCTCTTATGCATGCTGACAGACTAACAGACTGTTCCTTT |
| | CCTGGGTCTTTTCTGCAGGCCTGTACGGAAGTGTTACTTC |
| | TGCTCTAAAAGCTGCGGAATTGTACCCGCGGCCGATCCAA |
| | TCGATACAGATCTAGCGGCCGCCCGCCACCATGGCTGAGA |
| | AAGAGTCAACATCACCACACCTCATGGTTCCCATTCTTCT |
| | CCTGGTTGGATGGATTGTAGGCTGCATCATCGTTATTTAC |
| | ATTGTCTTCTTCTAAAAGCTTTGGATCCAATCAACCTCTG |
| | GATTACAAAATTTGTGAAAGATTGACTGGTATTCTTAACT |
| | ATGTTGCTCCTTTTACGCTATGTGGATACGCTGCTTTAAT |
| | GCCTTTGTATCATGCTATTGCTTCCCGTATGGCTTTCATT |
| | TTCTCCTCCTTGTATAAATCCTGGTTGCTGTCTCTTTATG |
| | AGGAGTTGTGGCCCGTTGTCAGGCAACGTGGCGTGGTGTG |
| | CACTGTGTTTGCTGACGCAACCCCCACTGGTTGGGGCATT |
| | GCCACCACCTGTCAGCTCCTTTCCGGGACTTTCGCTTTCC |
| | CCCTCCCTATTGCCACGGCGGAACTCATCGCCGCCTGCCT |
| | TGCCCGCTGCTGGACAGGGGCTCGGCTGTTGGGCACTGAC |
| | AATTCCGTGGTGTTGTCGGGGAAATCATCGTCCTTTCCTT |
| | GGCTGCTCGCCTGTGTTGCCACCTGGATTCTGCGCGGGAC |
| | GTCCTTCTGCTACGTCCCTTCGGCCCTCAATCCAGCGGAC |
| | CTTCCTTCCCGCGGCCTGCTGCCGGCTCTGCGGCCTCTTC |
| | CGCGTCTTCGAGATCTGCCTCGACTGTGCCTTCTAGTTGC |
| | CAGCCATCTGTTGTTTGCCCCTCCCCCGTGCCTTCCTTGA |
| | CCCTGGAAGGTGCCACTCCCACTGTCCTTTCCTAATAAAA |
| | TGAGGAAATTGCATCGCATTGTCTGAGTAGGTGTCATTCT |
| | ATTCTGGGGGGTGGGGTGGGGCAGGACAGCAAGGGGGAGG |
| | ATTGGGAAGACAATAGCAGGCATGCTGGGGACTCGAGTTA |
| | AGGGCAGCCAGAAGTCAGATGCTCAAGGGGCTTCATGATG |
| | TCCCCATAATTTTTGGCAGAGGGAAAAAGATCGGATCCTC |
| | AGGCGTAGTTCACCCCGTCCTCGAGGCCGCCCGGGTCGAC |
| | TAAAAAACCTCCCACACCTCCCCCTGAACCTGAAACATAA |
| | AATGAATGCAATTGTTGTTGTTAACTTGTTTATTGCAGCT |
| | TATAATGGTTACAAATAAAGCAATAGCATCACAAATTTCA |
| | CAAATAAAGCATTTTTTTCACTGCATTCTAGTTGTGGTTT |
| | GTCCAAACTCATCAATGTATCTTATCATGTCTGGATCCGC |
| | GCGGCCGTCAGAAGAACACGATGTAGATCACGATGATGCA |
| | GCCGACGATCCAGCCCACCAGCAGCAGAATAGGCACCATC |
| | AGGTGGGGGCTGGTAGATTCCTTCTCGGCCATGGTGGCGG |
| | CTAGCCTATAGTGAGTCGTATTAAGTACTCTAGCCTTAAG |
| | AGCTGTAATTGAACTGGGAGTGGACACCTGTGGAGAGAAA |
| | GGCAAAGTGGATGTCAGTAAGACCAATAGGTGCCTATCAG |
| | AAACGCAAGAGTCTTCTCTGTCTCGACAAGCCCAGTTTCT |
| | ATTGGTCTCCTTAAACCTGTCTTGTAACCTTGATACTTAC |
| | CTGCCCAGTGCCTCACGACCAACTTCTGCAGCTTAAGTTC |
| | GAGACTGTTGTGTCAGAAGCACTGACTGCGTTAGCAATTT |
| | AACTGTGATAAACTACCGCAATAAAGCTCTAGAGCTTCGG |
| | GGATCGTCCCACGGAGCGGTGGGTGCCGGCGGCTGTCTGG |
| | GAAGGGCTCCTTGGGGGGCAGAGGCTTTAAGGTCCCCCCG |
| | GCGCCCACCCCGGGGGGGGCAGAGCCAGCAGGAATGTGCC |
| | CGGCGCCCAGAGAGGAATGCAACACTTGTGAGCTGCTATT |
| | TTGGCAGCAGCGGCCCCGGCCCCCTCCGTGCTCCCCCTTC |
| | CCCCACAGGAGCCCATATAAGCCCAAGCTATTGTGTGGCC |
| | TCAGAGTTTTGCTATTTTAAACCCGTCGGACGGAGATACG |
| | TGAGTGCCCGAGGGGCTGACACAAGCCAGCCAGCTGTCAC |
| | CTCCCAGGGCTGGGGACGCTGATAAGGCAGCGCTTCGGAC |
| | CCGACCCTCTGCCGCAGCCCCAGATGCTGTCATGTGAAAG |
| | CCCAGACTGCTTTTATCCCTGCTTGGACTTCTAATGCTGT |
| | GCTTTTCCTTCAGTTCACACCAGTTAAAAATAGAAAACTG |
| | GGCACCGGCATCTCTGTCTAGAGTGGGCTTGGATTGATAT |
| | GCTGAGCAGGACTGCTGATTATCTTCAGGGGCCGGGCAGT |
| | GCTGGGTCCTCCCCTCAGAGCTTCTCAACCATGTTTGGGA |
| | TGGTCTAGGGATGAAACTGCTGGACAGCTGAGCCATGGAG |
| | ACTGGGGAGAGGCCTGCCTGGCTGAGACCAAGCCCTGCTG |
| | TACCATCTGCATTTGACAGTGAGGATGCCCATTGACAGGA |
| | AGCAGACGTAGAGAAAAGGAGAATCAGAATGATCGACAGG |
| | CTTCAAATCCTACCTCTAACACTTAACTACGTTCCGCACG |
| | TTTGGTCTCGGGCAGGCCAGTTGAATTCCCGATTAGGATC |
| | TTCCTAGAGCATGGCTACGTAGATAAGTAGCATGGCGGGT |
| | TAATCATTAACTACAAGGAACCCCTAGTGATGGAGTTGGC |
| | CACTCCCTCTCTGCGCGCTCGCTCGCTCACTGAGGCCGGG |
| | CGACCAAAGGTCGCCCGACGCCCGGGCTTTGCCCGGGCGG |
| | CCTCAGTGAGCGAGCGAGCGCGCAG (SEQ ID NO: 55) |
| pHZ33 | CTGCGCGCTCGCTCGCTCACTGAGGCCGCCCGGGCAAAGC |
| distinct sequence elements | CCGGGCGTCGGGCGACCTTTGGTCGCCCGGCCTCAGTGAG |
| are indicated in bold, | CGAGCGAGCGCGCAGAGAGGGAGTGGCCAACTCCATCACT |
| starting with ITR, | AGGGGTTCCTTGTAGTTAATGATTAACCCGCCATGCTACT |
| promoter/enhancer combo | TATCTACGTAGCCATGCTCTAGGAAGATCGGAATTCGCCC |
| (αMHCe, followed by | TTAAGCCTTCAGATTAAAAATAACTAAGGTAAGGGCCATG |
| hcTnTp), DWORF (which | TGGGTAGGGGAGGTGGTGTGAGACGGTCCTGTCTCTCCTC |
| is also underlined), WPRE, | TATCTGCCCATCGGCCCTTTGGGGAGGAGGAATGTGCCCA |
| polyA (hGHpA), | AGGACTAAAAAAAGGCCCTGGAGCCAGAGGGGCGAGGGCA |
| promoter/enhancer combo | GCAGACCTTTCATGGGCAAACCTCAGGGCTGCTGTCGTCA |
| (ACTC1e, followed by | TGGAGAAGACCCACCTTGCAGATGTCCTCACTGGGGCTGG |
| ccTnTp), optimized | CAGAGCCGGCAACCTGCCTAAGGCTGCTCAGTCCATTAGG |
| DWORF (which is also | AGCCAGTAGCCTGGAAGATGTCTTTACCCCCAGCATCAGT |
| underlined), SV40pA, ITR | TCAAGTGGAGCAGCACATAACTCTTGCCCTCTGCCTTCCA |
| | AGATTCTGGTGCTGAGACTTATGGAGTGTCTTGGAGGTTG |
| | CCTTCTGCCCCCCAACCCTGCTCCCAGCTGGCCCTCCCAG |
| | GCCTGGGTTGCTGGCCTCTGCTTTATCAGGATTCTCAAGA |
| | GGGACAGCTGGTTTATGTTGCATGACTGTTCCCTGCATAT |
| | CTGCTCTGGTTTTAAATAGCTTATCTGAGCAGCTGGAGGA |
| | CCACATGGGCTTATATGGCGTGGGGTACATGTTCCTGTAG |
| | CCTTGTCCCTGGCACCTGCCAAAATAGCAGCCAACACCCC |
| | CCACCCCCACCGCCATCCCCCTGCCCCACCCGTCCCCTGT |
| | CGCACATTCCTCCCTCCGCAGGGCTGGCTCACCAGGCCCC |
| | AGCCCACATGCCTGCTTAAAGCCCTCTCCATCCTCTGCCT |
| | CACCCAGTCCCCGCTGAGACTGAGCAGACGCCTCCATAAC |
| | TGGTAAGTACCGCCTATAGACTCTATAGGCACACCCCTTT |
| | GGCTCTTATGCATGCTGACAGACTAACAGACTGTTCCTTT |
| | CCTGGGTCTTTTCTGCAGGCCTGTACGGAAGTGTTACTTC |
| | TGCTCTAAAAGCTGCGGAATTGTACCCGCGGCCGATCCAA |
| | TCGATACAGATCTAGCGGCCGCCCGCCACCATGGCTGAGA |
| | AAGAGTCAACATCACCACACCTCATGGTTCCCATTCTTCT |
| | CCTGGTTGGATGGATTGTAGGCTGCATCATCGTTATTTAC |
| | ATTGTCTTCTTCTAAAAGCTTTGGATCCAATCAACCTCTG |
| | GATTACAAAATTTGTGAAAGATTGACTGGTATTCTTAACT |
| | ATGTTGCTCCTTTTACGCTATGTGGATACGCTGCTTTAAT |
| | GCCTTTGTATCATGCTATTGCTTCCCGTATGGCTTTCATT |
| | TTCTCCTCCTTGTATAAATCCTGGTTGCTGTCTCTTTATG |
| | AGGAGTTGTGGCCCGTTGTCAGGCAACGTGGCGTGGTGTG |
| | CACTGTGTTTGCTGACGCAACCCCCACTGGTTGGGGCATT |
| | GCCACCACCTGTCAGCTCCTTTCCGGGACTTTCGCTTTCC |
| | CCCTCCCTATTGCCACGGCGGAACTCATCGCCGCCTGCCT |
| | TGCCCGCTGCTGGACAGGGGCTCGGCTGTTGGGCACTGAC |
| | AATTCCGTGGTGTTGTCGGGGAAATCATCGTCCTTTCCTT |
| | GGCTGCTCGCCTGTGTTGCCACCTGGATTCTGCGCGGGAC |
| | GTCCTTCTGCTACGTCCCTTCGGCCCTCAATCCAGCGGAC |
| | CTTCCTTCCCGCGGCCTGCTGCCGGCTCTGCGGCCTCTTC |
| | CGCGTCTTCGAGATCTGCCTCGACTGTGCCTTCTAGTTGC |
| | CAGCCATCTGTTGTTTGCCCCTCCCCCGTGCCTTCCTTGA |
| | CCCTGGAAGGTGCCACTCCCACTGTCCTTTCCTAATAAAA |
| | TGAGGAAATTGCATCGCATTGTCTGAGTAGGTGTCATTCT |
| | ATTCTGGGGGGTGGGGTGGGGCAGGACAGCAAGGGGGAGG |
| | ATTGGGAAGACAATAGCAGGCATGCTGGGGACTCGAGTTA |
| | AGGGCAGCCAGAAGTCAGATGCTCAAGGGGCTTCATGATG |
| | TCCCCATAATTTTTGGCAGAGGGAAAAAGATCGGATCCTC |
| | AGGCGTAGTTCACCCCGTCCTCGAGGCCGCCCGGGTCGAC |
| | TAAAAAACCTCCCACACCTCCCCCTGAACCTGAAACATAA |
| | AATGAATGCAATTGTTGTTGTTAAACTGGCCTGCCCGAGA |
| | CCAAACGTGCGGAACGTAGTTAAGTGTTAGAGGTAGGATT |
| | TGAAGCCTGTCGATCATTCTGATTCTCCTTTTCTCTACGT |
| | CTGCTTCCTGTCAATGGGCATCCTCACTGTCAAATGCAGA |
| | TGGTACAGCAGGGCTTGGTCTCAGCCAGGCAGGCCTCTCC |
| | CCAGTCTCCATGGCTCAGCTGTCCAGCAGTTTCATCCCTA |
| | GACCATCCCAAACATGGTTGAGAAGCTCTGAGGGGAGGAC |
| | CCAGCACTGCCCGGCCCCTGAAGATAATCAGCAGTCCTGC |
| | TCAGCATATCAATCCAAGCCCACTCTAGACAGAGATGCCG |
| | GTGCCCAGTTTTCTATTTTTAACTGGTGTGAACTGAAGGA |
| | AAAGCACAGCATTAGAAGTCCAAGCAGGGATAAAAGCAGT |
| | CTGGGCTTTCACATGACAGCATCTGGGGCTGCGGCAGAGG |
| | GTCGGGTCCGAAGCGCTGCCTTATCAGCGTCCCCAGCCCT |
| | GGGAGGTGACAGCTGGCTGGCTTGTGTCAGCCCCTCGGGC |
| | ACTCACGTATCTCCGTCCGACGGGTTTAAAATAGCAAAAC |
| | TCTGAGGCCACACAATAGCTTGGGCTTATATGGGCTCCTG |
| | TGGGGGAAGGGGGAGCACGGAGGGGGCCGGGGCCGCTGCT |
| | GCCAAAATAGCAGCTCACAAGTGTTGCATTCCTCTCTGGG |
| | CGCCGGGCACATTCCTGCTGGCTCTGCCCGCCCCGGGGTG |
| | GGCGCCGGGGGGACCTTAAAGCCTCTGCCCCCCAAGGAGC |
| | CCTTCCCAGACAGCCGCCGGCACCCACCGCTCCGTGGGAC |
| | GATCCCCGAAGCTCTAGAGCTTTATTGCGGTAGTTTATCA |
| | CAGTTAAATTGCTAACGCAGTCAGTGCTTCTGACACAACA |
| | GTCTCGAACTTAAGCTGCAGAAGTTGGTCGTGAGGCACTG |
| | GGCAGGTAAGTATCAAGGTTACAAGACAGGTTTAAGGAGA |
| | CCAATAGAAACTGGGCTTGTCGAGACAGAGAAGACTCTTG |
| | CGTTTCTGATAGGCACCTATTGGTCTTACTGACATCCACT |
| | TTGCCTTTCTCTCCACAGGTGTCCACTCCCAGTTCAATTA |
| | CAGCTCTTAAGGCTAGAGTACTTAATACGACTCACTATAG |
| | GCTAGCCGCCACCATGGCCGAGAAGGAATCTACCAGCCCC |
| | CACCTGATGGTGCCTATTCTGCTGCTGGTGGGCTGGATCG |
| | TCGGCTGCATCATCGTGATCTACATCGTGTTCTTCTGACG |
| | GCCGCGCGGATCCAGACATGATAAGATACATTGATGAGTT |
| | TGGACAAACCACAACTAGAATGCAGTGAAAAAAATGCTTT |
| | ATTTGTGAAATTTGTGATGCTATTGCTTTATTTGTAACCA |
| | TTATAAGCTGCAATAAACAAGTGAATTCCCGATTAGGATC |
| | TTCCTAGAGCATGGCTACGTAGATAAGTAGCATGGCGGGT |
| | TAATCATTAACTACAAGGAACCCCTAGTGATGGAGTTGGC |
| | CACTCCCTCTCTGCGCGCTCGCTCGCTCACTGAGGCCGGG |
| | CGACCAAAGGTCGCCCGACGCCCGGGCTTTGCCCGGGCGG |
| | CCTCAGTGAGCGAGCGAGCGCGCAG (SEQ ID NO: 56) |
| pHZ34 | CTGCGCGCTCGCTCGCTCACTGAGGCCGCCCGGGCAAAGC |
| distinct sequence elements | CCGGGCGTCGGGCGACCTTTGGTCGCCCGGCCTCAGTGAG |
| are indicated in bold, | CGAGCGAGCGCGCAGAGAGGGAGTGGCCAACTCCATCACT |
| starting with ITR, polyA | AGGGGTTCCTTGTAGTTAATGATTAACCCGCCATGCTACT |
| (hGHpA), WPRE, DWORF | TATCTACGTAGCCATGCTCTAGGAAGATCGGAATTCTCCC |
| (which is also underlined), | CAGCATGCCTGCTATTGTCTTCCCAATCCTCCCCCTTGCT |
| promoter/enhancer combo | GTCCTGCCCCACCCCACCCCCCAGAATAGAATGACACCTA |
| (hcTnTp, followed by | CTCAGACAATGCGATGCAATTTCCTCATTTTATTAGGAAA |
| αMHCe, followed by | GGACAGTGGGAGTGGCACCTTCCAGGGTCAAGGAAGGCAC |
| ACTC1e, followed by | GGGGGAGGGGCAAACAACAGATGGCTGGCAACTAGAAGGC |
| ccTnTp), optimized | ACAGTCGAGGCAGATCTCGAAGACGCGGAAGAGGCCGCAG |
| DWORF (which is also | AGCCGGCAGCAGGCCGCGGGAAGGAAGGTCCGCTGGATTG |
| underlined), SV40pA, ITR | AGGGCCGAAGGGACGTAGCAGAAGGACGTCCCGCGCAGAA |
| | TCCAGGTGGCAACACAGGCGAGCAGCCAAGGAAAGGACGA |
| | TGATTTCCCCGACAACACCACGGAATTGTCAGTGCCCAAC |
| | AGCCGAGCCCCTGTCCAGCAGCGGGCAAGGCAGGCGGCGA |
| | TGAGTTCCGCCGTGGCAATAGGGAGGGGGAAAGCGAAAGT |
| | CCCGGAAAGGAGCTGACAGGTGGTGGCAATGCCCCAACCA |
| | GTGGGGGTTGCGTCAGCAAACACAGTGCACACCACGCCAC |
| | GTTGCCTGACAACGGGCCACAACTCCTCATAAAGAGACAG |
| | CAACCAGGATTTATACAAGGAGGAGAAAATGAAAGCCATA |
| | CGGGAAGCAATAGCATGATACAAAGGCATTAAAGCAGCGT |
| | ATCCACATAGCGTAAAAGGAGCAACATAGTTAAGAATACC |
| | AGTCAATCTTTCACAAATTTTGTAATCCAGAGGTTGATTG |
| | GATCCAAAGCTTTTAGAAGAAGACAATGTAAATAACGATG |
| | ATGCAGCCTACAATCCATCCAACCAGGAGAAGAATGGGAA |
| | CCATGAGGTGTGGTGATGTTGACTCTTTCTCAGCCATGGT |
| | GGCGGGCGGCCGCTAGATCTGTATCGATTGGATCGGCCGC |
| | GGGTACAATTCCGCAGCTTTTAGAGCAGAAGTAACACTTC |
| | CGTACAGGCCTGCAGAAAAGACCCAGGAAAGGAACAGTCT |
| | GTTAGTCTGTCAGCATGCATAAGAGCCAAAGGGGTGTGCC |
| | TATAGAGTCTATAGGCGGTACTTACCAGTTATGGAGGCGT |
| | CTGCTCAGTCTCAGCGGGGACTGGGTGAGGCAGAGGATGG |
| | AGAGGGCTTTAAGCAGGCATGTGGGCTGGGGCCTGGTGAG |
| | CCAGCCCTGCGGAGGGAGGAATGTGCGACAGGGGACGGGT |
| | GGGGCAGGGGGATGGCGGTGGGGGTGGGGGGTGTTGGCTG |
| | CTATTTTGGCAGGTGCCAGGGACAAGGCTACAGGAACATG |
| | TACCCCACGCCATATAAGCCCATGTGGTCCTCCAGCTGCT |
| | CAGATAAGCTATTTAAAACCAGAGCAGATATGCAGGGAAC |
| | AGTCATGCAACATAAACCAGCTGTCCCTCTTGAGAATCCT |
| | GATAAAGCAGAGGCCAGCAACCCAGGCCTGGGAGGGCCAG |
| | CTGGGAGCAGGGTTGGGGGGCAGAAGGCAACCTCCAAGAC |
| | ACTCCATAAGTCTCAGCACCAGAATCTTGGAAGGCAGAGG |
| | GCAAGAGTTATGTGCTGCTCCACTTGAACTGATGCTGGGG |
| | GTAAAGACATCTTCCAGGCTACTGGCTCCTAATGGACTGA |
| | GCAGCCTTAGGCAGGTTGCCGGCTCTGCCAGCCCCAGTGA |
| | GGACATCTGCAAGGTGGGTCTTCTCCATGACGACAGCAGC |
| | CCTGAGGTTTGCCCATGAAAGGTCTGCTGCCCTCGCCCCT |
| | CTGGCTCCAGGGCCTTTTTTTAGTCCTTGGGCACATTCCT |
| | CCTCCCCAAAGGGCCGATGGGCAGATAGAGGAGAGACAGG |
| | ACCGTCTCACACCACCTCCCCTACCCACATGGCCCTTACC |
| | TTAGTTATTTTTAATCTGAAGGCTCGAGTTAAGGGCAGCC |
| | AGAAGTCAGATGCTCAAGGGGCTTCATGATGTCCCCATAA |
| | TTTTTGGCAGAGGGAAAAAGATCGGATCCTCAGGCGTAGT |
| | TCACCCCGTCCTCGAGGCCGCCCGGGTCGACTAAAAAACC |
| | TCCCACACCTCCCCCTGAACCTGAAACATAAAATGAATGC |
| | AATTGTTGTTGTTAAACTGGCCTGCCCGAGACCAAACGTG |
| | CGGAACGTAGTTAAGTGTTAGAGGTAGGATTTGAAGCCTG |
| | TCGATCATTCTGATTCTCCTTTTCTCTACGTCTGCTTCCT |
| | GTCAATGGGCATCCTCACTGTCAAATGCAGATGGTACAGC |
| | AGGGCTTGGTCTCAGCCAGGCAGGCCTCTCCCCAGTCTCC |
| | ATGGCTCAGCTGTCCAGCAGTTTCATCCCTAGACCATCCC |
| | AAACATGGTTGAGAAGCTCTGAGGGGAGGACCCAGCACTG |
| | CCCGGCCCCTGAAGATAATCAGCAGTCCTGCTCAGCATAT |
| | CAATCCAAGCCCACTCTAGACAGAGATGCCGGTGCCCAGT |
| | TTTCTATTTTTAACTGGTGTGAACTGAAGGAAAAGCACAG |
| | CATTAGAAGTCCAAGCAGGGATAAAAGCAGTCTGGGCTTT |
| | CACATGACAGCATCTGGGGCTGCGGCAGAGGGTCGGGTCC |
| | GAAGCGCTGCCTTATCAGCGTCCCCAGCCCTGGGAGGTGA |
| | CAGCTGGCTGGCTTGTGTCAGCCCCTCGGGCACTCACGTA |
| | TCTCCGTCCGACGGGTTTAAAATAGCAAAACTCTGAGGCC |
| | ACACAATAGCTTGGGCTTATATGGGCTCCTGTGGGGGAAG |
| | GGGGAGCACGGAGGGGGCCGGGGCCGCTGCTGCCAAAATA |
| | GCAGCTCACAAGTGTTGCATTCCTCTCTGGGCGCCGGGCA |
| | CATTCCTGCTGGCTCTGCCCGCCCCGGGGTGGGCGCCGGG |
| | GGGACCTTAAAGCCTCTGCCCCCCAAGGAGCCCTTCCCAG |
| | ACAGCCGCCGGCACCCACCGCTCCGTGGGACGATCCCCGA |
| | AGCTCTAGAGCTTTATTGCGGTAGTTTATCACAGTTAAAT |
| | TGCTAACGCAGTCAGTGCTTCTGACACAACAGTCTCGAAC |
| | TTAAGCTGCAGAAGTTGGTCGTGAGGCACTGGGCAGGTAA |
| | GTATCAAGGTTACAAGACAGGTTTAAGGAGACCAATAGAA |
| | ACTGGGCTTGTCGAGACAGAGAAGACTCTTGCGTTTCTGA |
| | TAGGCACCTATTGGTCTTACTGACATCCACTTTGCCTTTC |
| | TCTCCACAGGTGTCCACTCCCAGTTCAATTACAGCTCTTA |
| | AGGCTAGAGTACTTAATACGACTCACTATAGGCTAGCCGC |
| | CACCATGGCCGAGAAGGAATCTACCAGCCCCCACCTGATG |
| | GTGCCTATTCTGCTGCTGGTGGGCTGGATCGTCGGCTGCA |
| | TCATCGTGATCTACATCGTGTTCTTCTGACGGCCGCGCGG |
| | ATCCAGACATGATAAGATACATTGATGAGTTTGGACAAAC |
| | CACAACTAGAATGCAGTGAAAAAAATGCTTTATTTGTGAA |
| | ATTTGTGATGCTATTGCTTTATTTGTAACCATTATAAGCT |
| | GCAATAAACAAGTGAATTCCCGATTAGGATCTTCCTAGAG |
| | CATGGCTACGTAGATAAGTAGCATGGCGGGTTAATCATTA |
| | ACTACAAGGAACCCCTAGTGATGGAGTTGGCCACTCCCTC |
| | TCTGCGCGCTCGCTCGCTCACTGAGGCCGGGCGACCAAAG |
| | GTCGCCCGACGCCCGGGCTTTGCCCGGGCGGCCTCAGTGA |
| | GCGAGCGAGCGCGCAG (SEQ ID NO: 57) |
| pHZ69 | CTGCGCGCTCGCTCGCTCACTGAGGCCGCCCGGGCAAAGC |
| distinct sequence elements | CCGGGCGTCGGGCGACCTTTGGTCGCCCGGCCTCAGTGAG |
| are indicated in bold, | CGAGCGAGCGCGCAGAGAGGGAGTGGCCAACTCCATCACT |
| starting with ITR, hcTnTp, | AGGGGTTCCTTGTAGTTAATGATTAACCCGCCATGCTACT |
| DWORF (which is also | TATCTACGTAGCCATGCTCTAGGAAGATCGGAATTCGCCC |
| underlined), WPRE, polyA | TTAAGGTCATGGAGAAGACCCACCTTGCAGATGTCCTCAC |
| (hGHpA), SV40pA, | TGGGGCTGGCAGAGCCGGCAACCTGCCTAAGGCTGCTCAG |
| optimized DWORF (which | TCCATTAGGAGCCAGTAGCCTGGAAGATGTCTTTACCCCC |
| is also underlined), ccTnTp, | AGCATCAGTTCAAGTGGAGCAGCACATAACTCTTGCCCTC |
| ITR | TGCCTTCCAAGATTCTGGTGCTGAGACTTATGGAGTGTCT |
| | TGGAGGTTGCCTTCTGCCCCCCAACCCTGCTCCCAGCTGG |
| | CCCTCCCAGGCCTGGGTTGCTGGCCTCTGCTTTATCAGGA |
| | TTCTCAAGAGGGACAGCTGGTTTATGTTGCATGACTGTTC |
| | CCTGCATATCTGCTCTGGTTTTAAATAGCTTATCTGAGCA |
| | GCTGGAGGACCACATGGGCTTATATGGCGTGGGGTACATG |
| | TTCCTGTAGCCTTGTCCCTGGCACCTGCCAAAATAGCAGC |
| | CAACACCCCCCACCCCCACCGCCATCCCCCTGCCCCACCC |
| | GTCCCCTGTCGCACATTCCTCCCTCCGCAGGGCTGGCTCA |
| | CCAGGCCCCAGCCCACATGCCTGCTTAAAGCCCTCTCCAT |
| | CCTCTGCCTCACCCAGTCCCCGCTGAGACTGAGCAGACGC |
| | CTCCATAACTGGTAAGTACCGCCTATAGACTCTATAGGCA |
| | CACCCCTTTGGCTCTTATGCATGCTGACAGACTAACAGAC |
| | TGTTCCTTTCCTGGGTCTTTTCTGCAGGCCTGTACGGAAG |
| | TGTTACTTCTGCTCTAAAAGCTGCGGAATTGTACCCGCGG |
| | CCGATCCAATCGATACAGATCTAGCGGCCGCCCGCCACCA |
| | TGGCTGAGAAAGAGTCAACATCACCACACCTCATGGTTCC |
| | CATTCTTCTCCTGGTTGGATGGATTGTAGGCTGCATCATC |
| | GTTATTTACATTGTCTTCTTCTAAAAGCTTTGGATCCAAT |
| | CAACCTCTGGATTACAAAATTTGTGAAAGATTGACTGGTA |
| | TTCTTAACTATGTTGCTCCTTTTACGCTATGTGGATACGC |
| | TGCTTTAATGCCTTTGTATCATGCTATTGCTTCCCGTATG |
| | GCTTTCATTTTCTCCTCCTTGTATAAATCCTGGTTGCTGT |
| | CTCTTTATGAGGAGTTGTGGCCCGTTGTCAGGCAACGTGG |
| | CGTGGTGTGCACTGTGTTTGCTGACGCAACCCCCACTGGT |
| | TGGGGCATTGCCACCACCTGTCAGCTCCTTTCCGGGACTT |
| | TCGCTTTCCCCCTCCCTATTGCCACGGCGGAACTCATCGC |
| | CGCCTGCCTTGCCCGCTGCTGGACAGGGGCTCGGCTGTTG |
| | GGCACTGACAATTCCGTGGTGTTGTCGGGGAAATCATCGT |
| | CCTTTCCTTGGCTGCTCGCCTGTGTTGCCACCTGGATTCT |
| | GCGCGGGACGTCCTTCTGCTACGTCCCTTCGGCCCTCAAT |
| | CCAGCGGACCTTCCTTCCCGCGGCCTGCTGCCGGCTCTGC |
| | GGCCTCTTCCGCGTCTTCGAGATCTGCCTCGACTGTGCCT |
| | TCTAGTTGCCAGCCATCTGTTGTTTGCCCCTCCCCCGTGC |
| | CTTCCTTGACCCTGGAAGGTGCCACTCCCACTGTCCTTTC |
| | CTAATAAAATGAGGAAATTGCATCGCATTGTCTGAGTAGG |
| | TGTCATTCTATTCTGGGGGGTGGGGTGGGGCAGGACAGCA |
| | AGGGGGAGGATTGGGAAGACAATAGCAGGCATGCTGGGGA |
| | CTCGAGTTAAGGGCAGCCAGAAGTCAGATGCTCAAGGGGC |
| | TTCATGATGTCCCCATAATTTTTGGCAGAGGGAAAAAGAT |
| | CGGATCCTCAGGCGTAGTTCACCCCGTCCTCGAGGCCGCC |
| | CGGGTCGACTAAAAAACCTCCCACACCTCCCCCTGAACCT |
| | GAAACATAAAATGAATGCAATTGTTGTTGTTAACTTGTTT |
| | ATTGCAGCTTATAATGGTTACAAATAAAGCAATAGCATCA |
| | CAAATTTCACAAATAAAGCATTTTTTTCACTGCATTCTAG |
| | TTGTGGTTTGTCCAAACTCATCAATGTATCTTATCATGTC |
| | TGGATCCGCGCGGCCGTCAGAAGAACACGATGTAGATCAC |
| | GATGATGCAGCCGACGATCCAGCCCACCAGCAGCAGAATA |
| | GGCACCATCAGGTGGGGGCTGGTAGATTCCTTCTCGGCCA |
| | TGGTGGCGGCTAGCCTATAGTGAGTCGTATTAAGTACTCT |
| | AGCCTTAAGAGCTGTAATTGAACTGGGAGTGGACACCTGT |
| | GGAGAGAAAGGCAAAGTGGATGTCAGTAAGACCAATAGGT |
| | GCCTATCAGAAACGCAAGAGTCTTCTCTGTCTCGACAAGC |
| | CCAGTTTCTATTGGTCTCCTTAAACCTGTCTTGTAACCTT |
| | GATACTTACCTGCCCAGTGCCTCACGACCAACTTCTGCAG |
| | CTTAAGTTCGAGACTGTTGTGTCAGAAGCACTGACTGCGT |
| | TAGCAATTTAACTGTGATAAACTACCGCAATAAAGCTCTA |
| | GAGCTTCGGGGATCGTCCCACGGAGCGGTGGGTGCCGGCG |
| | GCTGTCTGGGAAGGGCTCCTTGGGGGGCAGAGGCTTTAAG |
| | GTCCCCCCGGCGCCCACCCCGGGGGGGGCAGAGCCAGCAG |
| | GAATGTGCCCGGCGCCCAGAGAGGAATGCAACACTTGTGA |
| | GCTGCTATTTTGGCAGCAGCGGCCCCGGCCCCCTCCGTGC |
| | TCCCCCTTCCCCCACAGGAGCCCATATAAGCCCAAGCTAT |
| | TGTGTGGCCTCAGAGTTTTGCTATTTTAAACCCGTCGGAC |
| | GGAGATACGTGAGTGCCCGAGGGGCTGACACAAGCCAGCC |
| | AGCTGTCACCTCCCAGGGCTGGGGACGCTGATAAGGCAGC |
| | GCTTCGGACCCGACCCTCTGCCGCAGCCCCAGATGCTGTC |
| | ATGTGAAAGCCCAGACTGCTTTTATCCCGAATTCCCGATT |
| | AGGATCTTCCTAGAGCATGGCTACGTAGATAAGTAGCATG |
| | GCGGGTTAATCATTAACTACAAGGAACCCCTAGTGATGGA |
| | GTTGGCCACTCCCTCTCTGCGCGCTCGCTCGCTCACTGAG |
| | GCCGGGCGACCAAAGGTCGCCCGACGCCCGGGCTTTGCCC |
| | GGGCGGCCTCAGTGAGCGAGCGAGCGCGCAG |
| | (SEQ ID NO: 58) |
| pHZ72 | CTGCGCGCTCGCTCGCTCACTGAGGCCGCCCGGGCAAAGC |
| distinct sequence elements | CCGGGCGTCGGGCGACCTTTGGTCGCCCGGCCTCAGTGAG |
| are indicated in bold, | CGAGCGAGCGCGCAGAGAGGGAGTGGCCAACTCCATCACT |
| starting with ITR, hcTnTp, | AGGGGTTCCTTGTAGTTAATGATTAACCCGCCATGCTACT |
| DWORF (which is also | TATCTACGTAGCCATGCTCTAGGAAGATCGGAATTCGCCC |
| underlined), WPRE, polyA | TTAAGGTCATGGAGAAGACCCACCTTGCAGATGTCCTCAC |
| (hGHpA), ccTnTp, | TGGGGCTGGCAGAGCCGGCAACCTGCCTAAGGCTGCTCAG |
| optimized DWORF (which | TCCATTAGGAGCCAGTAGCCTGGAAGATGTCTTTACCCCC |
| is also underlined), | AGCATCAGTTCAAGTGGAGCAGCACATAACTCTTGCCCTC |
| SV40pA, ITR | TGCCTTCCAAGATTCTGGTGCTGAGACTTATGGAGTGTCT |
| | TGGAGGTTGCCTTCTGCCCCCCAACCCTGCTCCCAGCTGG |
| | CCCTCCCAGGCCTGGGTTGCTGGCCTCTGCTTTATCAGGA |
| | TTCTCAAGAGGGACAGCTGGTTTATGTTGCATGACTGTTC |
| | CCTGCATATCTGCTCTGGTTTTAAATAGCTTATCTGAGCA |
| | GCTGGAGGACCACATGGGCTTATATGGCGTGGGGTACATG |
| | TTCCTGTAGCCTTGTCCCTGGCACCTGCCAAAATAGCAGC |
| | CAACACCCCCCACCCCCACCGCCATCCCCCTGCCCCACCC |
| | GTCCCCTGTCGCACATTCCTCCCTCCGCAGGGCTGGCTCA |
| | CCAGGCCCCAGCCCACATGCCTGCTTAAAGCCCTCTCCAT |
| | CCTCTGCCTCACCCAGTCCCCGCTGAGACTGAGCAGACGC |
| | CTCCATAACTGGTAAGTACCGCCTATAGACTCTATAGGCA |
| | CACCCCTTTGGCTCTTATGCATGCTGACAGACTAACAGAC |
| | TGTTCCTTTCCTGGGTCTTTTCTGCAGGCCTGTACGGAAG |
| | TGTTACTTCTGCTCTAAAAGCTGCGGAATTGTACCCGCGG |
| | CCGATCCAATCGATACAGATCTAGCGGCCGCCCGCCACCA |
| | TGGCTGAGAAAGAGTCAACATCACCACACCTCATGGTTCC |
| | CATTCTTCTCCTGGTTGGATGGATTGTAGGCTGCATCATC |
| | GTTATTTACATTGTCTTCTTCTAAAAGCTTTGGATCCAAT |
| | CAACCTCTGGATTACAAAATTTGTGAAAGATTGACTGGTA |
| | TTCTTAACTATGTTGCTCCTTTTACGCTATGTGGATACGC |
| | TGCTTTAATGCCTTTGTATCATGCTATTGCTTCCCGTATG |
| | GCTTTCATTTTCTCCTCCTTGTATAAATCCTGGTTGCTGT |
| | CTCTTTATGAGGAGTTGTGGCCCGTTGTCAGGCAACGTGG |
| | CGTGGTGTGCACTGTGTTTGCTGACGCAACCCCCACTGGT |
| | TGGGGCATTGCCACCACCTGTCAGCTCCTTTCCGGGACTT |
| | TCGCTTTCCCCCTCCCTATTGCCACGGCGGAACTCATCGC |
| | CGCCTGCCTTGCCCGCTGCTGGACAGGGGCTCGGCTGTTG |
| | GGCACTGACAATTCCGTGGTGTTGTCGGGGAAATCATCGT |
| | CCTTTCCTTGGCTGCTCGCCTGTGTTGCCACCTGGATTCT |
| | GCGCGGGACGTCCTTCTGCTACGTCCCTTCGGCCCTCAAT |
| | CCAGCGGACCTTCCTTCCCGCGGCCTGCTGCCGGCTCTGC |
| | GGCCTCTTCCGCGTCTTCGAGATCTGCCTCGACTGTGCCT |
| | TCTAGTTGCCAGCCATCTGTTGTTTGCCCCTCCCCCGTGC |
| | CTTCCTTGACCCTGGAAGGTGCCACTCCCACTGTCCTTTC |
| | CTAATAAAATGAGGAAATTGCATCGCATTGTCTGAGTAGG |
| | TGTCATTCTATTCTGGGGGGTGGGGTGGGGCAGGACAGCA |
| | AGGGGGAGGATTGGGAAGACAATAGCAGGCATGCTGGGGA |
| | CTCGAGTTAAGGGCAGCCAGAAGTCAGATGCTCAAGGGGC |
| | TTCATGATGTCCCCATAATTTTTGGCAGAGGGAAAAAGAT |
| | CGGATCCTCAGGCGTAGTTCACCCCGTCCTCGAGGCCGCC |
| | CGGGTCGACTAAAAAACCTCCCACACCTCCCCCTGAACCT |
| | GAAACATAAAATGAATGCAATTGTTGTTGTTAGGGATAAA |
| | AGCAGTCTGGGCTTTCACATGACAGCATCTGGGGCTGCGG |
| | CAGAGGGTCGGGTCCGAAGCGCTGCCTTATCAGCGTCCCC |
| | AGCCCTGGGAGGTGACAGCTGGCTGGCTTGTGTCAGCCCC |
| | TCGGGCACTCACGTATCTCCGTCCGACGGGTTTAAAATAG |
| | CAAAACTCTGAGGCCACACAATAGCTTGGGCTTATATGGG |
| | CTCCTGTGGGGGAAGGGGGAGCACGGAGGGGGCCGGGGCC |
| | GCTGCTGCCAAAATAGCAGCTCACAAGTGTTGCATTCCTC |
| | TCTGGGCGCCGGGCACATTCCTGCTGGCTCTGCCCGCCCC |
| | GGGGTGGGCGCCGGGGGGACCTTAAAGCCTCTGCCCCCCA |
| | AGGAGCCCTTCCCAGACAGCCGCCGGCACCCACCGCTCCG |
| | TGGGACGATCCCCGAAGCTCTAGAGCTTTATTGCGGTAGT |
| | TTATCACAGTTAAATTGCTAACGCAGTCAGTGCTTCTGAC |
| | ACAACAGTCTCGAACTTAAGCTGCAGAAGTTGGTCGTGAG |
| | GCACTGGGCAGGTAAGTATCAAGGTTACAAGACAGGTTTA |
| | AGGAGACCAATAGAAACTGGGCTTGTCGAGACAGAGAAGA |
| | CTCTTGCGTTTCTGATAGGCACCTATTGGTCTTACTGACA |
| | TCCACTTTGCCTTTCTCTCCACAGGTGTCCACTCCCAGTT |
| | CAATTACAGCTCTTAAGGCTAGAGTACTTAATACGACTCA |
| | CTATAGGCTAGCCGCCACCATGGCCGAGAAGGAATCTACC |
| | AGCCCCCACCTGATGGTGCCTATTCTGCTGCTGGTGGGCT |
| | GGATCGTCGGCTGCATCATCGTGATCTACATCGTGTTCTT |
| | CTGACGGCCGCGCGGATCCAGACATGATAAGATACATTGA |
| | TGAGTTTGGACAAACCACAACTAGAATGCAGTGAAAAAAA |
| | TGCTTTATTTGTGAAATTTGTGATGCTATTGCTTTATTTG |
| | TAACCATTATAAGCTGCAATAAACAAGTGAATTCCCGATT |
| | AGGATCTTCCTAGAGCATGGCTACGTAGATAAGTAGCATG |
| | GCGGGTTAATCATTAACTACAAGGAACCCCTAGTGATGGA |
| | GTTGGCCACTCCCTCTCTGCGCGCTCGCTCGCTCACTGAG |
| | GCCGGGCGACCAAAGGTCGCCCGACGCCCGGGCTTTGCCC |
| | GGGCGGCCTCAGTGAGCGAGCGAGCGCGCAG |
| | (SEQ ID NO: 59) |
| pHZ75 | CTGCGCGCTCGCTCGCTCACTGAGGCCGCCCGGGCAAAGC |
| distinct sequence elements | CCGGGCGTCGGGCGACCTTTGGTCGCCCGGCCTCAGTGAG |
| are indicated in bold, | CGAGCGAGCGCGCAGAGAGGGAGTGGCCAACTCCATCACT |
| starting with ITR, polyA | AGGGGTTCCTTGTAGTTAATGATTAACCCGCCATGCTACT |
| (hGHpA), WPRE, | TATCTACGTAGCCATGCTCTAGGAAGATCGGAATTCTCCC |
| DWORF(which is also | CAGCATGCCTGCTATTGTCTTCCCAATCCTCCCCCTTGCT |
| underlined), hcTnTp/ | GTCCTGCCCCACCCCACCCCCCAGAATAGAATGACACCTA |
| ccTnTp combo, optimized | CTCAGACAATGCGATGCAATTTCCTCATTTTATTAGGAAA |
| DWORF (which is also | GGACAGTGGGAGTGGCACCTTCCAGGGTCAAGGAAGGCAC |
| underlined), SV40pA, ITR | GGGGGAGGGGCAAACAACAGATGGCTGGCAACTAGAAGGC |
| | ACAGTCGAGGCAGATCTCGAAGACGCGGAAGAGGCCGCAG |
| | AGCCGGCAGCAGGCCGCGGGAAGGAAGGTCCGCTGGATTG |
| | AGGGCCGAAGGGACGTAGCAGAAGGACGTCCCGCGCAGAA |
| | TCCAGGTGGCAACACAGGCGAGCAGCCAAGGAAAGGACGA |
| | TGATTTCCCCGACAACACCACGGAATTGTCAGTGCCCAAC |
| | AGCCGAGCCCCTGTCCAGCAGCGGGCAAGGCAGGCGGCGA |
| | TGAGTTCCGCCGTGGCAATAGGGAGGGGGAAAGCGAAAGT |
| | CCCGGAAAGGAGCTGACAGGTGGTGGCAATGCCCCAACCA |
| | GTGGGGGTTGCGTCAGCAAACACAGTGCACACCACGCCAC |
| | GTTGCCTGACAACGGGCCACAACTCCTCATAAAGAGACAG |
| | CAACCAGGATTTATACAAGGAGGAGAAAATGAAAGCCATA |
| | CGGGAAGCAATAGCATGATACAAAGGCATTAAAGCAGCGT |
| | ATCCACATAGCGTAAAAGGAGCAACATAGTTAAGAATACC |
| | AGTCAATCTTTCACAAATTTTGTAATCCAGAGGTTGATTG |
| | GATCCAAAGCTTTTAGAAGAAGACAATGTAAATAACGATG |
| | ATGCAGCCTACAATCCATCCAACCAGGAGAAGAATGGGAA |
| | CCATGAGGTGTGGTGATGTTGACTCTTTCTCAGCCATGGT |
| | GGCGGGCGGCCGCTAGATCTGTATCGATTGGATCGGCCGC |
| | GGGTACAATTCCGCAGCTTTTAGAGCAGAAGTAACACTTC |
| | CGTACAGGCCTGCAGAAAAGACCCAGGAAAGGAACAGTCT |
| | GTTAGTCTGTCAGCATGCATAAGAGCCAAAGGGGTGTGCC |
| | TATAGAGTCTATAGGCGGTACTTACCAGTTATGGAGGCGT |
| | CTGCTCAGTCTCAGCGGGGACTGGGTGAGGCAGAGGATGG |
| | AGAGGGCTTTAAGCAGGCATGTGGGCTGGGGCCTGGTGAG |
| | CCAGCCCTGCGGAGGGAGGAATGTGCGACAGGGGACGGGT |
| | GGGGCAGGGGGATGGCGGTGGGGGTGGGGGGTGTTGGCTG |
| | CTATTTTGGCAGGTGCCAGGGACAAGGCTACAGGAACATG |
| | TACCCCACGCCATATAAGCCCATGTGGTCCTCCAGCTGCT |
| | CAGATAAGCTATTTAAAACCAGAGCAGATATGCAGGGAAC |
| | AGTCATGCAACATAAACCAGCTGTCCCTCTTGAGAATCCT |
| | GATAAAGCAGAGGCCAGCAACCCAGGCCTGGGAGGGCCAG |
| | CTGGGAGCAGGGTTGGGGGGCAGAAGGCAACCTCCAAGAC |
| | ACTCCATAAGTCTCAGCACCAGAATCTTGGAAGGCAGAGG |
| | GCAAGAGTTATGTGCTGCTCCACTTGAACTGATGCTGGGG |
| | GTAAAGACATCTTCCAGGCTACTGGCTCCTAATGGACTGA |
| | GCAGCCTTAGGCAGGTTGCCGGCTCTGCCAGCCCCAGTGA |
| | GGACATCTGCAAGGTGGGTCTTCTCCATGACCTCGAGTTA |
| | AGGGCAGCCAGAAGTCAGATGCTCAAGGGGCTTCATGATG |
| | TCCCCATAATTTTTGGCAGAGGGAAAAAGATCGGATCCTC |
| | AGGCGTAGTTCACCCCGTCCTCGAGGCCGCCCGGGTCGAC |
| | TAAAAAACCTCCCACACCTCCCCCTGAACCTGAAACATAA |
| | AATGAATGCAATTGTTGTTGTTAGGGATAAAAGCAGTCTG |
| | GGCTTTCACATGACAGCATCTGGGGCTGCGGCAGAGGGTC |
| | GGGTCCGAAGCGCTGCCTTATCAGCGTCCCCAGCCCTGGG |
| | AGGTGACAGCTGGCTGGCTTGTGTCAGCCCCTCGGGCACT |
| | CACGTATCTCCGTCCGACGGGTTTAAAATAGCAAAACTCT |
| | GAGGCCACACAATAGCTTGGGCTTATATGGGCTCCTGTGG |
| | GGGAAGGGGGAGCACGGAGGGGGCCGGGGCCGCTGCTGCC |
| | AAAATAGCAGCTCACAAGTGTTGCATTCCTCTCTGGGCGC |
| | CGGGCACATTCCTGCTGGCTCTGCCCGCCCCGGGGTGGGC |
| | GCCGGGGGGACCTTAAAGCCTCTGCCCCCCAAGGAGCCCT |
| | TCCCAGACAGCCGCCGGCACCCACCGCTCCGTGGGACGAT |
| | CCCCGAAGCTCTAGAGCTTTATTGCGGTAGTTTATCACAG |
| | TTAAATTGCTAACGCAGTCAGTGCTTCTGACACAACAGTC |
| | TCGAACTTAAGCTGCAGAAGTTGGTCGTGAGGCACTGGGC |
| | AGGTAAGTATCAAGGTTACAAGACAGGTTTAAGGAGACCA |
| | ATAGAAACTGGGCTTGTCGAGACAGAGAAGACTCTTGCGT |
| | TTCTGATAGGCACCTATTGGTCTTACTGACATCCACTTTG |
| | CCTTTCTCTCCACAGGTGTCCACTCCCAGTTCAATTACAG |
| | CTCTTAAGGCTAGAGTACTTAATACGACTCACTATAGGCT |
| | AGCCGCCACCATGGCCGAGAAGGAATCTACCAGCCCCCAC |
| | CTGATGGTGCCTATTCTGCTGCTGGTGGGCTGGATCGTCG |
| | GCTGCATCATCGTGATCTACATCGTGTTCTTCTGACGGCC |
| | GCGCGGATCCAGACATGATAAGATACATTGATGAGTTTGG |
| | ACAAACCACAACTAGAATGCAGTGAAAAAAATGCTTTATT |
| | TGTGAAATTTGTGATGCTATTGCTTTATTTGTAACCATTA |
| | TAAGCTGCAATAAACAAGTGAATTCCCGATTAGGATCTTC |
| | CTAGAGCATGGCTACGTAGATAAGTAGCATGGCGGGTTAA |
| | TCATTAACTACAAGGAACCCCTAGTGATGGAGTTGGCCAC |
| | TCCCTCTCTGCGCGCTCGCTCGCTCACTGAGGCCGGGCGA |
| | CCAAAGGTCGCCCGACGCCCGGGCTTTGCCCGGGCGGCCT |
| | CAGTGAGCGAGCGAGCGCGCAG (SEQ ID NO: 60) |
| pHZ51 (human DWORF | CTGCGCGCTCGCTCGCTCACTGAGGCCGCCCGGGCAAAGC |
| version of pHZ15) | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGAACTGGCCTGCCCGAGACCAAACGTGCGGAACGTA |
| | GTTAAGTGTTAGAGGTAGGATTTGAAGCCTGTCGATCATT |
| | CTGATTCTCCTTTTCTCTACGTCTGCTTCCTGTCAATGGG |
| | CATCCTCACTGTCAAATGCAGATGGTACAGCAGGGCTTGG |
| | TCTCAGCCAGGCAGGCCTCTCCCCAGTCTCCATGGCTCAG |
| | CTGTCCAGCAGTTTCATCCCTAGACCATCCCAAACATGGT |
| | TGAGAAGCTCTGAGGGGAGGACCCAGCACTGCCCGGCCCC |
| | TGAAGATAATCAGCAGTCCTGCTCAGCATATCAATCCAAG |
| | CCCACTCTAGACAGAGATGCCGGTGCCCAGTTTTCTATTT |
| | TTAACTGGTGTGAACTGAAGGAAAAGCACAGCATTAGAAG |
| | TCCAAGCAGTCATGGAGAAGACCCACCTTGCAGATGTCCT |
| | CACTGGGGCTGGCAGAGCCGGCAACCTGCCTAAGGCTGCT |
| | CAGTCCATTAGGAGCCAGTAGCCTGGAAGATGTCTTTACC |
| | CCCAGCATCAGTTCAAGTGGAGCAGCACATAACTCTTGCC |
| | CTCTGCCTTCCAAGATTCTGGTGCTGAGACTTATGGAGTG |
| | TCTTGGAGGTTGCCTTCTGCCCCCCAACCCTGCTCCCAGC |
| | TGGCCCTCCCAGGCCTGGGTTGCTGGCCTCTGCTTTATCA |
| | GGATTCTCAAGAGGGACAGCTGGTTTATGTTGCATGACTG |
| | TTCCCTGCATATCTGCTCTGGTTTTAAATAGCTTATCTGA |
| | GCAGCTGGAGGACCACATGGGCTTATATGGCGTGGGGTAC |
| | ATGTTCCTGTAGCCTTGTCCCTGGCACCTGCCAAAATAGC |
| | AGCCAACACCCCCCACCCCCACCGCCATCCCCCTGCCCCA |
| | CCCGTCCCCTGTCGCACATTCCTCCCTCCGCAGGGCTGGC |
| | TCACCAGGCCCCAGCCCACATGCCTGCTTAAAGCCCTCTC |
| | CATCCTCTGCCTCACCCAGTCCCCGCTGAGACTGAGCAGA |
| | CGCCTCCAGCGGCCGCCCGCCACCATGGCTGAAAAAGCGG |
| | GGTCTACATTTTCACACCTTCTGGTTCCTATTCTTCTCCT |
| | GATTGGCTGGATTGTGGGCTGCATCATAATGATTTATGTT |
| | GTCTTCTCTTAGAAGCTTTGGATCCAATCAACCTCTGGAT |
| | TACAAAATTTGTGAAAGATTGACTGGTATTCTTAACTATG |
| | TTGCTCCTTTTACGCTATGTGGATACGCTGCTTTAATGCC |
| | TTTGTATCATGCTATTGCTTCCCGTATGGCTTTCATTTTC |
| | TCCTCCTTGTATAAATCCTGGTTGCTGTCTCTTTATGAGG |
| | AGTTGTGGCCCGTTGTCAGGCAACGTGGCGTGGTGTGCAC |
| | TGTGTTTGCTGACGCAACCCCCACTGGTTGGGGCATTGCC |
| | ACCACCTGTCAGCTCCTTTCCGGGACTTTCGCTTTCCCCC |
| | TCCCTATTGCCACGGCGGAACTCATCGCCGCCTGCCTTGC |
| | CCGCTGCTGGACAGGGGCTCGGCTGTTGGGCACTGACAAT |
| | TCCGTGGTGTTGTCGGGGAAATCATCGTCCTTTCCTTGGC |
| | TGCTCGCCTGTGTTGCCACCTGGATTCTGCGCGGGACGTC |
| | CTTCTGCTACGTCCCTTCGGCCCTCAATCCAGCGGACCTT |
| | CCTTCCCGCGGCCTGCTGCCGGCTCTGCGGCCTCTTCCGC |
| | GTCTTCGAGATCTGCCTCGACTGTGCCTTCTAGTTGCCAG |
| | CCATCTGTTGTTTGCCCCTCCCCCGTGCCTTCCTTGACCC |
| | TGGAAGGTGCCACTCCCACTGTCCTTTCCTAATAAAATGA |
| | GGAAATTGCATCGCATTGTCTGAGTAGGTGTCATTCTATT |
| | CTGGGGGGTGGGGTGGGGCAGGACAGCAAGGGGGAGGATT |
| | GGGAAGACAATAGCAGGCATGCTGGGGACTCGAGTTAAGG |
| | GCGAATTCCCGATTAGGATCTTCCTAGAGCATGGCTACGT |
| | AGATAAGTAGCATGGCGGGTTAATCATTAACTACAAGGAA |
| | CCCCTAGTGATGGAGTTGGCCACTCCCTCTCTGCGCGCTC |
| | GCTCGCTCACTGAGGCCGGGCGACCAAAGGTCGCCCGACG |
| | CCCGGGCTTTGCCCGGGCGGCCTCAGTGAGCGAGCGAGCG |
| | CGCAG (SEQ ID NO: 61) |
| pHZ100 (human DWORF | CTGCGCGCTCGCTCGCTCACTGAGGCCGCCCGGGCAAAGC |
| version of pHZ72) | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGGTCATGGAGAAGACCCACCTTGCAGATGTCCTCAC |
| | TGGGGCTGGCAGAGCCGGCAACCTGCCTAAGGCTGCTCAG |
| | TCCATTAGGAGCCAGTAGCCTGGAAGATGTCTTTACCCCC |
| | AGCATCAGTTCAAGTGGAGCAGCACATAACTCTTGCCCTC |
| | TGCCTTCCAAGATTCTGGTGCTGAGACTTATGGAGTGTCT |
| | TGGAGGTTGCCTTCTGCCCCCCAACCCTGCTCCCAGCTGG |
| | CCCTCCCAGGCCTGGGTTGCTGGCCTCTGCTTTATCAGGA |
| | TTCTCAAGAGGGACAGCTGGTTTATGTTGCATGACTGTTC |
| | CCTGCATATCTGCTCTGGTTTTAAATAGCTTATCTGAGCA |
| | GCTGGAGGACCACATGGGCTTATATGGCGTGGGGTACATG |
| | TTCCTGTAGCCTTGTCCCTGGCACCTGCCAAAATAGCAGC |
| | CAACACCCCCCACCCCCACCGCCATCCCCCTGCCCCACCC |
| | GTCCCCTGTCGCACATTCCTCCCTCCGCAGGGCTGGCTCA |
| | CCAGGCCCCAGCCCACATGCCTGCTTAAAGCCCTCTCCAT |
| | CCTCTGCCTCACCCAGTCCCCGCTGAGACTGAGCAGACGC |
| | CTCCATAACTGGTAAGTACCGCCTATAGACTCTATAGGCA |
| | CACCCCTTTGGCTCTTATGCATGCTGACAGACTAACAGAC |
| | TGTTCCTTTCCTGGGTCTTTTCTGCAGGCCTGTACGGAAG |
| | TGTTACTTCTGCTCTAAAAGCTGCGGAATTGTACCCGCGG |
| | CCGATCCAATCGATACAGATCTAGCGGCCGCCCGCCACCA |
| | TGGCTGAAAAAGCGGGGTCTACATTTTCACACCTTCTGGT |
| | TCCTATTCTTCTCCTGATTGGCTGGATTGTGGGCTGCATC |
| | ATAATGATTTATGTTGTCTTCTCTTAGAAGCTTTGGATCC |
| | AATCAACCTCTGGATTACAAAATTTGTGAAAGATTGACTG |
| | GTATTCTTAACTATGTTGCTCCTTTTACGCTATGTGGATA |
| | CGCTGCTTTAATGCCTTTGTATCATGCTATTGCTTCCCGT |
| | ATGGCTTTCATTTTCTCCTCCTTGTATAAATCCTGGTTGC |
| | TGTCTCTTTATGAGGAGTTGTGGCCCGTTGTCAGGCAACG |
| | TGGCGTGGTGTGCACTGTGTTTGCTGACGCAACCCCCACT |
| | GGTTGGGGCATTGCCACCACCTGTCAGCTCCTTTCCGGGA |
| | CTTTCGCTTTCCCCCTCCCTATTGCCACGGCGGAACTCAT |
| | CGCCGCCTGCCTTGCCCGCTGCTGGACAGGGGCTCGGCTG |
| | TTGGGCACTGACAATTCCGTGGTGTTGTCGGGGAAATCAT |
| | CGTCCTTTCCTTGGCTGCTCGCCTGTGTTGCCACCTGGAT |
| | TCTGCGCGGGACGTCCTTCTGCTACGTCCCTTCGGCCCTC |
| | AATCCAGCGGACCTTCCTTCCCGCGGCCTGCTGCCGGCTC |
| | TGCGGCCTCTTCCGCGTCTTCGAGATCTGCCTCGACTGTG |
| | CCTTCTAGTTGCCAGCCATCTGTTGTTTGCCCCTCCCCCG |
| | TGCCTTCCTTGACCCTGGAAGGTGCCACTCCCACTGTCCT |
| | TTCCTAATAAAATGAGGAAATTGCATCGCATTGTCTGAGT |
| | AGGTGTCATTCTATTCTGGGGGGTGGGGTGGGGCAGGACA |
| | GCAAGGGGGAGGATTGGGAAGACAATAGCAGGCATGCTGG |
| | GGACTCGAGTTAAGGGCAGCCAGAAGTCAGATGCTCAAGG |
| | GGCTTCATGATGTCCCCATAATTTTTGGCAGAGGGAAAAA |
| | GATCGGATCCTCAGGCGTAGTTCACCCCGTCCTCGAGGCC |
| | GCCCGGGTCGACTAAAAAACCTCCCACACCTCCCCCTGAA |
| | CCTGAAACATAAAATGAATGCAATTGTTGTTGTTAGGGAT |
| | AAAAGCAGTCTGGGCTTTCACATGACAGCATCTGGGGCTG |
| | CGGCAGAGGGTCGGGTCCGAAGCGCTGCCTTATCAGCGTC |
| | CCCAGCCCTGGGAGGTGACAGCTGGCTGGCTTGTGTCAGC |
| | CCCTCGGGCACTCACGTATCTCCGTCCGACGGGTTTAAAA |
| | TAGCAAAACTCTGAGGCCACACAATAGCTTGGGCTTATAT |
| | GGGCTCCTGTGGGGGAAGGGGGAGCACGGAGGGGGCCGGG |
| | GCCGCTGCTGCCAAAATAGCAGCTCACAAGTGTTGCATTC |
| | CTCTCTGGGCGCCGGGCACATTCCTGCTGGCTCTGCCCGC |
| | CCCGGGGTGGGCGCCGGGGGGACCTTAAAGCCTCTGCCCC |
| | CCAAGGAGCCCTTCCCAGACAGCCGCCGGCACCCACCGCT |
| | CCGTGGGACGATCCCCGAAGCTCTAGAGCTTTATTGCGGT |
| | AGTTTATCACAGTTAAATTGCTAACGCAGTCAGTGCTTCT |
| | GACACAACAGTCTCGAACTTAAGCTGCAGAAGTTGGTCGT |
| | GAGGCACTGGGCAGGTAAGTATCAAGGTTACAAGACAGGT |
| | TTAAGGAGACCAATAGAAACTGGGCTTGTCGAGACAGAGA |
| | AGACTCTTGCGTTTCTGATAGGCACCTATTGGTCTTACTG |
| | ACATCCACTTTGCCTTTCTCTCCACAGGTGTCCACTCCCA |
| | GTTCAATTACAGCTCTTAAGGCTAGAGTACTTAATACGAC |
| | TCACTATAGGCTAGCCGCCACCATGGCCGAGAAGGCCGGA |
| | TCTACCTTCAGCCACCTGCTGGTCCCTATTCTGCTGCTGA |
| | TCGGCTGGATCGTGGGCTGCATCATCATGATCTACGTGGT |
| | GTTCAGCTGACGGCCGCGCGGATCCAGACATGATAAGATA |
| | CATTGATGAGTTTGGACAAACCACAACTAGAATGCAGTGA |
| | AAAAAATGCTTTATTTGTGAAATTTGTGATGCTATTGCTT |
| | TATTTGTAACCATTATAAGCTGCAATAAACAAGTGAATTC |
| | CCGATTAGGATCTTCCTAGAGCATGGCTACGTAGATAAGT |
| | AGCATGGCGGGTTAATCATTAACTACAAGGAACCCCTAGT |
| | GATGGAGTTGGCCACTCCCTCTCTGCGCGCTCGCTCGCTC |
| | ACTGAGGCCGGGCGACCAAAGGTCGCCCGACGCCCGGGCT |
| | TTGCCCGGGCGGCCTCAGTGAGCGAGCGAGCGCGCAG |
| | (SEQ ID NO: 62) |
| pHZ101 (human DWORF | CTGCGCGCTCGCTCGCTCACTGAGGCCGCCCGGGCAAAGC |
| version of pHZ75) | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCTCCC |
| | CAGCATGCCTGCTATTGTCTTCCCAATCCTCCCCCTTGCT |
| | GTCCTGCCCCACCCCACCCCCCAGAATAGAATGACACCTA |
| | CTCAGACAATGCGATGCAATTTCCTCATTTTATTAGGAAA |
| | GGACAGTGGGAGTGGCACCTTCCAGGGTCAAGGAAGGCAC |
| | GGGGGAGGGGCAAACAACAGATGGCTGGCAACTAGAAGGC |
| | ACAGTCGAGGCAGATCTCGAAGACGCGGAAGAGGCCGCAG |
| | AGCCGGCAGCAGGCCGCGGGAAGGAAGGTCCGCTGGATTG |
| | AGGGCCGAAGGGACGTAGCAGAAGGACGTCCCGCGCAGAA |
| | TCCAGGTGGCAACACAGGCGAGCAGCCAAGGAAAGGACGA |
| | TGATTTCCCCGACAACACCACGGAATTGTCAGTGCCCAAC |
| | AGCCGAGCCCCTGTCCAGCAGCGGGCAAGGCAGGCGGCGA |
| | TGAGTTCCGCCGTGGCAATAGGGAGGGGGAAAGCGAAAGT |
| | CCCGGAAAGGAGCTGACAGGTGGTGGCAATGCCCCAACCA |
| | GTGGGGGTTGCGTCAGCAAACACAGTGCACACCACGCCAC |
| | GTTGCCTGACAACGGGCCACAACTCCTCATAAAGAGACAG |
| | CAACCAGGATTTATACAAGGAGGAGAAAATGAAAGCCATA |
| | CGGGAAGCAATAGCATGATACAAAGGCATTAAAGCAGCGT |
| | ATCCACATAGCGTAAAAGGAGCAACATAGTTAAGAATACC |
| | AGTCAATCTTTCACAAATTTTGTAATCCAGAGGTTGATTG |
| | GATCCAAAGCTTCTAAGAGAAGACAACATAAATCATTATG |
| | ATGCAGCCCACAATCCAGCCAATCAGGAGAAGAATAGGAA |
| | CCAGAAGGTGTGAAAATGTAGACCCCGCTTTTTCAGCCAT |
| | GGTGGCGGGCGGCCGCTAGATCTGTATCGATTGGATCGGC |
| | CGCGGGTACAATTCCGCAGCTTTTAGAGCAGAAGTAACAC |
| | TTCCGTACAGGCCTGCAGAAAAGACCCAGGAAAGGAACAG |
| | TCTGTTAGTCTGTCAGCATGCATAAGAGCCAAAGGGGTGT |
| | GCCTATAGAGTCTATAGGCGGTACTTACCAGTTATGGAGG |
| | CGTCTGCTCAGTCTCAGCGGGGACTGGGTGAGGCAGAGGA |
| | TGGAGAGGGCTTTAAGCAGGCATGTGGGCTGGGGCCTGGT |
| | GAGCCAGCCCTGCGGAGGGAGGAATGTGCGACAGGGGACG |
| | GGTGGGGCAGGGGGATGGCGGTGGGGGTGGGGGGTGTTGG |
| | CTGCTATTTTGGCAGGTGCCAGGGACAAGGCTACAGGAAC |
| | ATGTACCCCACGCCATATAAGCCCATGTGGTCCTCCAGCT |
| | GCTCAGATAAGCTATTTAAAACCAGAGCAGATATGCAGGG |
| | AACAGTCATGCAACATAAACCAGCTGTCCCTCTTGAGAAT |
| | CCTGATAAAGCAGAGGCCAGCAACCCAGGCCTGGGAGGGC |
| | CAGCTGGGAGCAGGGTTGGGGGGCAGAAGGCAACCTCCAA |
| | GACACTCCATAAGTCTCAGCACCAGAATCTTGGAAGGCAG |
| | AGGGCAAGAGTTATGTGCTGCTCCACTTGAACTGATGCTG |
| | GGGGTAAAGACATCTTCCAGGCTACTGGCTCCTAATGGAC |
| | TGAGCAGCCTTAGGCAGGTTGCCGGCTCTGCCAGCCCCAG |
| | TGAGGACATCTGCAAGGTGGGTCTTCTCCATGACCTCGAG |
| | TTAAGGGCAGCCAGAAGTCAGATGCTCAAGGGGCTTCATG |
| | ATGTCCCCATAATTTTTGGCAGAGGGAAAAAGATCGGATC |
| | CTCAGGCGTAGTTCACCCCGTCCTCGAGGCCGCCCGGGTC |
| | GACTAAAAAACCTCCCACACCTCCCCCTGAACCTGAAACA |
| | TAAAATGAATGCAATTGTTGTTGTTAGGGATAAAAGCAGT |
| | CTGGGCTTTCACATGACAGCATCTGGGGCTGCGGCAGAGG |
| | GTCGGGTCCGAAGCGCTGCCTTATCAGCGTCCCCAGCCCT |
| | GGGAGGTGACAGCTGGCTGGCTTGTGTCAGCCCCTCGGGC |
| | ACTCACGTATCTCCGTCCGACGGGTTTAAAATAGCAAAAC |
| | TCTGAGGCCACACAATAGCTTGGGCTTATATGGGCTCCTG |
| | TGGGGGAAGGGGGAGCACGGAGGGGGCCGGGGCCGCTGCT |
| | GCCAAAATAGCAGCTCACAAGTGTTGCATTCCTCTCTGGG |
| | CGCCGGGCACATTCCTGCTGGCTCTGCCCGCCCCGGGGTG |
| | GGCGCCGGGGGGACCTTAAAGCCTCTGCCCCCCAAGGAGC |
| | CCTTCCCAGACAGCCGCCGGCACCCACCGCTCCGTGGGAC |
| | GATCCCCGAAGCTCTAGAGCTTTATTGCGGTAGTTTATCA |
| | CAGTTAAATTGCTAACGCAGTCAGTGCTTCTGACACAACA |
| | GTCTCGAACTTAAGCTGCAGAAGTTGGTCGTGAGGCACTG |
| | GGCAGGTAAGTATCAAGGTTACAAGACAGGTTTAAGGAGA |
| | CCAATAGAAACTGGGCTTGTCGAGACAGAGAAGACTCTTG |
| | CGTTTCTGATAGGCACCTATTGGTCTTACTGACATCCACT |
| | TTGCCTTTCTCTCCACAGGTGTCCACTCCCAGTTCAATTA |
| | CAGCTCTTAAGGCTAGAGTACTTAATACGACTCACTATAG |
| | GCTAGCCGCCACCATGGCCGAGAAGGCCGGATCTACCTTC |
| | AGCCACCTGCTGGTCCCTATTCTGCTGCTGATCGGCTGGA |
| | TCGTGGGCTGCATCATCATGATCTACGTGGTGTTCAGCTG |
| | ACGGCCGCGCGGATCCAGACATGATAAGATACATTGATGA |
| | GTTTGGACAAACCACAACTAGAATGCAGTGAAAAAAATGC |
| | TTTATTTGTGAAATTTGTGATGCTATTGCTTTATTTGTAA |
| | CCATTATAAGCTGCAATAAACAAGTGAATTCCCGATTAGG |
| | ATCTTCCTAGAGCATGGCTACGTAGATAAGTAGCATGGCG |
| | GGTTAATCATTAACTACAAGGAACCCCTAGTGATGGAGTT |
| | GGCCACTCCCTCTCTGCGCGCTCGCTCGCTCACTGAGGCC |
| | GGGCGACCAAAGGTCGCCCGACGCCCGGGCTTTGCCCGGG |
| | CGGCCTCAGTGAGCGAGCGAGCGCGCAG |
| | (SEQ ID NO: 63) |
| pHZ52 (human DWORF | CTGCGCGCTCGCTCGCTCACTGAGGCCGCCCGGGCAAAGC |
| version of pHZ16) | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGAACTGGCCTGCCCGAGACCAAACGTGCGGAACGTA |
| | GTTAAGTGTTAGAGGTAGGATTTGAAGCCTGTCGATCATT |
| | CTGATTCTCCTTTTCTCTACGTCTGCTTCCTGTCAATGGG |
| | CATCCTCACTGTCAAATGCAGATGGTACAGCAGGGCTTGG |
| | TCTCAGCCAGGCAGGCCTCTCCCCAGTCTCCATGGCTCAG |
| | CTGTCCAGCAGTTTCATCCCTAGACCATCCCAAACATGGT |
| | TGAGAAGCTCTGAGGGGAGGACCCAGCACTGCCCGGCCCC |
| | TGAAGATAATCAGCAGTCCTGCTCAGCATATCAATCCAAG |
| | CCCACTCTAGACAGAGATGCCGGTGCCCAGTTTTCTATTT |
| | TTAACTGGTGTGAACTGAAGGAAAAGCACAGCATTAGAAG |
| | TCCAAGCACCTTCAGATTAAAAATAACTAAGGTAAGGGCC |
| | ATGTGGGTAGGGGAGGTGGTGTGAGACGGTCCTGTCTCTC |
| | CTCTATCTGCCCATCGGCCCTTTGGGGAGGAGGAATGTGC |
| | CCAAGGACTAAAAAAAGGCCCTGGAGCCAGAGGGGCGAGG |
| | GCAGCAGACCTTTCATGGGCAAACCTCAGGGCTGCTGTCG |
| | TCATGGAGAAGACCCACCTTGCAGATGTCCTCACTGGGGC |
| | TGGCAGAGCCGGCAACCTGCCTAAGGCTGCTCAGTCCATT |
| | AGGAGCCAGTAGCCTGGAAGATGTCTTTACCCCCAGCATC |
| | AGTTCAAGTGGAGCAGCACATAACTCTTGCCCTCTGCCTT |
| | CCAAGATTCTGGTGCTGAGACTTATGGAGTGTCTTGGAGG |
| | TTGCCTTCTGCCCCCCAACCCTGCTCCCAGCTGGCCCTCC |
| | CAGGCCTGGGTTGCTGGCCTCTGCTTTATCAGGATTCTCA |
| | AGAGGGACAGCTGGTTTATGTTGCATGACTGTTCCCTGCA |
| | TATCTGCTCTGGTTTTAAATAGCTTATCTGAGCAGCTGGA |
| | GGACCACATGGGCTTATATGGCGTGGGGTACATGTTCCTG |
| | TAGCCTTGTCCCTGGCACCTGCCAAAATAGCAGCCAACAC |
| | CCCCCACCCCCACCGCCATCCCCCTGCCCCACCCGTCCCC |
| | TGTCGCACATTCCTCCCTCCGCAGGGCTGGCTCACCAGGC |
| | CCCAGCCCACATGCCTGCTTAAAGCCCTCTCCATCCTCTG |
| | CCTCACCCAGTCCCCGCTGAGACTGAGCAGACGCCTCCAG |
| | CGGCCGCCCGCCACCATGGCTGAAAAAGCGGGGTCTACAT |
| | TTTCACACCTTCTGGTTCCTATTCTTCTCCTGATTGGCTG |
| | GATTGTGGGCTGCATCATAATGATTTATGTTGTCTTCTCT |
| | TAGAAGCTTTGGATCCAATCAACCTCTGGATTACAAAATT |
| | TGTGAAAGATTGACTGGTATTCTTAACTATGTTGCTCCTT |
| | TTACGCTATGTGGATACGCTGCTTTAATGCCTTTGTATCA |
| | TGCTATTGCTTCCCGTATGGCTTTCATTTTCTCCTCCTTG |
| | TATAAATCCTGGTTGCTGTCTCTTTATGAGGAGTTGTGGC |
| | CCGTTGTCAGGCAACGTGGCGTGGTGTGCACTGTGTTTGC |
| | TGACGCAACCCCCACTGGTTGGGGCATTGCCACCACCTGT |
| | CAGCTCCTTTCCGGGACTTTCGCTTTCCCCCTCCCTATTG |
| | CCACGGCGGAACTCATCGCCGCCTGCCTTGCCCGCTGCTG |
| | GACAGGGGCTCGGCTGTTGGGCACTGACAATTCCGTGGTG |
| | TTGTCGGGGAAATCATCGTCCTTTCCTTGGCTGCTCGCCT |
| | GTGTTGCCACCTGGATTCTGCGCGGGACGTCCTTCTGCTA |
| | CGTCCCTTCGGCCCTCAATCCAGCGGACCTTCCTTCCCGC |
| | GGCCTGCTGCCGGCTCTGCGGCCTCTTCCGCGTCTTCGAG |
| | ATCTGCCTCGACTGTGCCTTCTAGTTGCCAGCCATCTGTT |
| | GTTTGCCCCTCCCCCGTGCCTTCCTTGACCCTGGAAGGTG |
| | CCACTCCCACTGTCCTTTCCTAATAAAATGAGGAAATTGC |
| | ATCGCATTGTCTGAGTAGGTGTCATTCTATTCTGGGGGGT |
| | GGGGTGGGGCAGGACAGCAAGGGGGAGGATTGGGAAGACA |
| | ATAGCAGGCATGCTGGGGACTCGAGTTAAGGGCGAATTCC |
| | CGATTAGGATCTTCCTAGAGCATGGCTACGTAGATAAGTA |
| | GCATGGCGGGTTAATCATTAACTACAAGGAACCCCTAGTG |
| | ATGGAGTTGGCCACTCCCTCTCTGCGCGCTCGCTCGCTCA |
| | CTGAGGCCGGGCGACCAAAGGTCGCCCGACGCCCGGGCTT |
| | TGCCCGGGCGGCCTCAGTGAGCGAGCGAGCGCGCAG |
| | (SEQ ID NO: 64) |
| pHZ53 (human DWORF | CTGCGCGCTCGCTCGCTCACTGAGGCCGCCCGGGCAAAGC |
| version of pHZ17) | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGCCTTCAGATTAAAAATAACTAAGGTAAGGGCCATG |
| | TGGGTAGGGGAGGTGGTGTGAGACGGTCCTGTCTCTCCTC |
| | TATCTGCCCATCGGCCCTTTGGGGAGGAGGAATGTGCCCA |
| | AGGACTAAAAAAAGGCCCTGGAGCCAGAGGGGCGAGGGCA |
| | GCAGACCTTTCATGGGCAAACCTCAGGGCTGCTGTCGTCA |
| | TGGAGAAGACCCACCTTGCAGATGTCCTCACTGGGGCTGG |
| | CAGAGCCGGCAACCTGCCTAAGGCTGCTCAGTCCATTAGG |
| | AGCCAGTAGCCTGGAAGATGTCTTTACCCCCAGCATCAGT |
| | TCAAGTGGAGCAGCACATAACTCTTGCCCTCTGCCTTCCA |
| | AGATTCTGGTGCTGAGACTTATGGAGTGTCTTGGAGGTTG |
| | CCTTCTGCCCCCCAACCCTGCTCCCAGCTGGCCCTCCCAG |
| | GCCTGGGTTGCTGGCCTCTGCTTTATCAGGATTCTCAAGA |
| | GGGACAGCTGGTTTATGTTGCATGACTGTTCCCTGCATAT |
| | CTGCTCTGGTTTTAAATAGCTTATCTGAGCAGCTGGAGGA |
| | CCACATGGGCTTATATGGCGTGGGGTACATGTTCCTGTAG |
| | CCTTGTCCCTGGCACCTGCCAAAATAGCAGCCAACACCCC |
| | CCACCCCCACCGCCATCCCCCTGCCCCACCCGTCCCCTGT |
| | CGCACATTCCTCCCTCCGCAGGGCTGGCTCACCAGGCCCC |
| | AGCCCACATGCCTGCTTAAAGCCCTCTCCATCCTCTGCCT |
| | CACCCAGTCCCCGCTGAGACTGAGCAGACGCCTCCAGCGG |
| | CCGCCCGCCACCATGGCTGAAAAAGCGGGGTCTACATTTT |
| | CACACCTTCTGGTTCCTATTCTTCTCCTGATTGGCTGGAT |
| | TGTGGGCTGCATCATAATGATTTATGTTGTCTTCTCTTAG |
| | AAGCTTTGGATCCAATCAACCTCTGGATTACAAAATTTGT |
| | GAAAGATTGACTGGTATTCTTAACTATGTTGCTCCTTTTA |
| | CGCTATGTGGATACGCTGCTTTAATGCCTTTGTATCATGC |
| | TATTGCTTCCCGTATGGCTTTCATTTTCTCCTCCTTGTAT |
| | AAATCCTGGTTGCTGTCTCTTTATGAGGAGTTGTGGCCCG |
| | TTGTCAGGCAACGTGGCGTGGTGTGCACTGTGTTTGCTGA |
| | CGCAACCCCCACTGGTTGGGGCATTGCCACCACCTGTCAG |
| | CTCCTTTCCGGGACTTTCGCTTTCCCCCTCCCTATTGCCA |
| | CGGCGGAACTCATCGCCGCCTGCCTTGCCCGCTGCTGGAC |
| | AGGGGCTCGGCTGTTGGGCACTGACAATTCCGTGGTGTTG |
| | TCGGGGAAATCATCGTCCTTTCCTTGGCTGCTCGCCTGTG |
| | TTGCCACCTGGATTCTGCGCGGGACGTCCTTCTGCTACGT |
| | CCCTTCGGCCCTCAATCCAGCGGACCTTCCTTCCCGCGGC |
| | CTGCTGCCGGCTCTGCGGCCTCTTCCGCGTCTTCGAGATC |
| | TGCCTCGACTGTGCCTTCTAGTTGCCAGCCATCTGTTGTT |
| | TGCCCCTCCCCCGTGCCTTCCTTGACCCTGGAAGGTGCCA |
| | CTCCCACTGTCCTTTCCTAATAAAATGAGGAAATTGCATC |
| | GCATTGTCTGAGTAGGTGTCATTCTATTCTGGGGGGTGGG |
| | GTGGGGCAGGACAGCAAGGGGGAGGATTGGGAAGACAATA |
| | GCAGGCATGCTGGGGACTCGAGTTAAGGGCGAATTCCCGA |
| | TTAGGATCTTCCTAGAGCATGGCTACGTAGATAAGTAGCA |
| | TGGCGGGTTAATCATTAACTACAAGGAACCCCTAGTGATG |
| | GAGTTGGCCACTCCCTCTCTGCGCGCTCGCTCGCTCACTG |
| | AGGCCGGGCGACCAAAGGTCGCCCGACGCCCGGGCTTTGC |
| | CCGGGCGGCCTCAGTGAGCGAGCGAGCGCGCAG |
| | (SEQ ID NO: 65) |
| pHZ54 (human DWORF | CTGCGCGCTCGCTCGCTCACTGAGGCCGCCCGGGCAAAGC |
| version of pHZ18) | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGCCTTCAGATTAAAAATAACTAAGGTAAGGGCCATG |
| | TGGGTAGGGGAGGTGGTGTGAGACGGTCCTGTCTCTCCTC |
| | TATCTGCCCATCGGCCCTTTGGGGAGGAGGAATGTGCCCA |
| | AGGACTAAAAAAAGGCCCTGGAGCCAGAGGGGCGAGGGCA |
| | GCAGACCTTTCATGGGCAAACCTCAGGGCTGCTGTCAACT |
| | GGCCTGCCCGAGACCAAACGTGCGGAACGTAGTTAAGTGT |
| | TAGAGGTAGGATTTGAAGCCTGTCGATCATTCTGATTCTC |
| | CTTTTCTCTACGTCTGCTTCCTGTCAATGGGCATCCTCAC |
| | TGTCAAATGCAGATGGTACAGCAGGGCTTGGTCTCAGCCA |
| | GGCAGGCCTCTCCCCAGTCTCCATGGCTCAGCTGTCCAGC |
| | AGTTTCATCCCTAGACCATCCCAAACATGGTTGAGAAGCT |
| | CTGAGGGGAGGACCCAGCACTGCCCGGCCCCTGAAGATAA |
| | TCAGCAGTCCTGCTCAGCATATCAATCCAAGCCCACTCTA |
| | GACAGAGATGCCGGTGCCCAGTTTTCTATTTTTAACTGGT |
| | GTGAACTGAAGGAAAAGCACAGCATTAGAAGTCCAAGCAG |
| | TCATGGAGAAGACCCACCTTGCAGATGTCCTCACTGGGGC |
| | TGGCAGAGCCGGCAACCTGCCTAAGGCTGCTCAGTCCATT |
| | AGGAGCCAGTAGCCTGGAAGATGTCTTTACCCCCAGCATC |
| | AGTTCAAGTGGAGCAGCACATAACTCTTGCCCTCTGCCTT |
| | CCAAGATTCTGGTGCTGAGACTTATGGAGTGTCTTGGAGG |
| | TTGCCTTCTGCCCCCCAACCCTGCTCCCAGCTGGCCCTCC |
| | CAGGCCTGGGTTGCTGGCCTCTGCTTTATCAGGATTCTCA |
| | AGAGGGACAGCTGGTTTATGTTGCATGACTGTTCCCTGCA |
| | TATCTGCTCTGGTTTTAAATAGCTTATCTGAGCAGCTGGA |
| | GGACCACATGGGCTTATATGGCGTGGGGTACATGTTCCTG |
| | TAGCCTTGTCCCTGGCACCTGCCAAAATAGCAGCCAACAC |
| | CCCCCACCCCCACCGCCATCCCCCTGCCCCACCCGTCCCC |
| | TGTCGCACATTCCTCCCTCCGCAGGGCTGGCTCACCAGGC |
| | CCCAGCCCACATGCCTGCTTAAAGCCCTCTCCATCCTCTG |
| | CCTCACCCAGTCCCCGCTGAGACTGAGCAGACGCCTCCAG |
| | CGGCCGCCCGCCACCATGGCTGAAAAAGCGGGGTCTACAT |
| | TTTCACACCTTCTGGTTCCTATTCTTCTCCTGATTGGCTG |
| | GATTGTGGGCTGCATCATAATGATTTATGTTGTCTTCTCT |
| | TAGAAGCTTTGGATCCAATCAACCTCTGGATTACAAAATT |
| | TGTGAAAGATTGACTGGTATTCTTAACTATGTTGCTCCTT |
| | TTACGCTATGTGGATACGCTGCTTTAATGCCTTTGTATCA |
| | TGCTATTGCTTCCCGTATGGCTTTCATTTTCTCCTCCTTG |
| | TATAAATCCTGGTTGCTGTCTCTTTATGAGGAGTTGTGGC |
| | CCGTTGTCAGGCAACGTGGCGTGGTGTGCACTGTGTTTGC |
| | TGACGCAACCCCCACTGGTTGGGGCATTGCCACCACCTGT |
| | CAGCTCCTTTCCGGGACTTTCGCTTTCCCCCTCCCTATTG |
| | CCACGGCGGAACTCATCGCCGCCTGCCTTGCCCGCTGCTG |
| | GACAGGGGCTCGGCTGTTGGGCACTGACAATTCCGTGGTG |
| | TTGTCGGGGAAATCATCGTCCTTTCCTTGGCTGCTCGCCT |
| | GTGTTGCCACCTGGATTCTGCGCGGGACGTCCTTCTGCTA |
| | CGTCCCTTCGGCCCTCAATCCAGCGGACCTTCCTTCCCGC |
| | GGCCTGCTGCCGGCTCTGCGGCCTCTTCCGCGTCTTCGAG |
| | ATCTGCCTCGACTGTGCCTTCTAGTTGCCAGCCATCTGTT |
| | GTTTGCCCCTCCCCCGTGCCTTCCTTGACCCTGGAAGGTG |
| | CCACTCCCACTGTCCTTTCCTAATAAAATGAGGAAATTGC |
| | ATCGCATTGTCTGAGTAGGTGTCATTCTATTCTGGGGGGT |
| | GGGGTGGGGCAGGACAGCAAGGGGGAGGATTGGGAAGACA |
| | ATAGCAGGCATGCTGGGGACTCGAGTTAAGGGCGAATTCC |
| | CGATTAGGATCTTCCTAGAGCATGGCTACGTAGATAAGTA |
| | GCATGGCGGGTTAATCATTAACTACAAGGAACCCCTAGTG |
| | ATGGAGTTGGCCACTCCCTCTCTGCGCGCTCGCTCGCTCA |
| | CTGAGGCCGGGCGACCAAAGGTCGCCCGACGCCCGGGCTT |
| | TGCCCGGGCGGCCTCAGTGAGCGAGCGAGCGCGCAG |
| | (SEQ ID NO: 66) |
| pHZ55 (human DWORF | CTGCGCGCTCGCTCGCTCACTGAGGCCGCCCGGGCAAAGC |
| version of pHZ19) | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGCCTTCAGATTAAAAATAACTAAGGTAAGGGCCATG |
| | TGGGTAGGGGAGGTGGTGTGAGACGGTCCTGTCTCTCCTC |
| | TATCTGCCCATCGGCCCTTTGGGGAGGAGGAATGTGCCCA |
| | AGGACTAAAAAAAGGCCCTGGAGCCAGAGGGGCGAGGGCA |
| | GCAGACCTTTCATGGGCAAACCTCAGGGCTGCTGTCAACT |
| | GGCCTGCCCGAGACCAAACGTGCGGAACGTAGTTAAGTGT |
| | TAGAGGTAGGATTTGAAGCCTGTCGATCATTCTGATTCTC |
| | CTTTTCTCTACGTCTGCTTCCTGTCAATGGGCATCCTCAC |
| | TGTCAAATGCAGATGGTACAGCAGGGCTTGGTCTCAGCCA |
| | GGCAGGCCTCTCCCCAGTCTCCATGGCTCAGCTGTCCAGC |
| | AGTTTCATCCCTAGACCATCCCAAACATGGTTGAGAAGCT |
| | CTGAGGGGAGGACCCAGCACTGCCCGGCCCCTGAAGATAA |
| | TCAGCAGTCCTGCTCAGCATATCAATCCAAGCCCACTCTA |
| | GACAGAGATGCCGGTGCCCAGTTTTCTATTTTTAACTGGT |
| | GTGAACTGAAGGAAAAGCACAGCATTAGAAGTCCAAGCAG |
| | TCATGGAGAAGACCCACCTTGCAGATGTCCTCACTGGGGC |
| | TGGCAGAGCCGGCAACCTGCCTAAGGCTGCTCAGTCCATT |
| | AGGAGCCAGTAGCCTGGAAGATGTCTTTACCCCCAGCATC |
| | AGTTCAAGTGGAGCAGCACATAACTCTTGCCCTCTGCCTT |
| | CCAAGATTCTGGTGCTGAGACTTATGGAGTGTCTTGGAGG |
| | TTGCCTTCTGCCCCCCAACCCTGCTCCCAGCTGGCCCTCC |
| | CAGGCCTGGGTTGCTGGCCTCTGCTTTATCAGGATTCTCA |
| | AGAGGGACAGCTGGTTTATGTTGCATGACTGTTCCCTGCA |
| | TATCTGCTCTGGTTTTAAATAGCTTATCTGAGCAGCTGGA |
| | GGACCACATGGGCTTATATGGCGTGGGGTACATGTTCCTG |
| | TAGCCTTGTCCCTGGCACCTGCCAAAATAGCAGCCAACAC |
| | CCCCCACCCCCACCGCCATCCCCCTGCCCCACCCGTCCCC |
| | TGTCGCACATTCCTCCCTCCGCAGGGCTGGCTCACCAGGC |
| | CCCAGCCCACATGCCTGCTTAAAGCCCTCTCCATCCTCTG |
| | CCTCACCCAGTCCCCGCTGAGACTGAGCAGACGCCTCCAT |
| | AACTGGTAAGTACCGCCTATAGACTCTATAGGCACACCCC |
| | TTTGGCTCTTATGCATGCTGACAGACTAACAGACTGTTCC |
| | TTTCCTGGGTCTTTTCTGCAGGCCTGTACGGAAGTGTTAC |
| | TTCTGCTCTAAAAGCTGCGGAATTGTACCCGCGGCCGATC |
| | CAATCGATACAGATCTAGCGGCCGCCCGCCACCATGGCTG |
| | AAAAAGCGGGGTCTACATTTTCACACCTTCTGGTTCCTAT |
| | TCTTCTCCTGATTGGCTGGATTGTGGGCTGCATCATAATG |
| | ATTTATGTTGTCTTCTCTTAGAAGCTTTGGATCCAATCAA |
| | CCTCTGGATTACAAAATTTGTGAAAGATTGACTGGTATTC |
| | TTAACTATGTTGCTCCTTTTACGCTATGTGGATACGCTGC |
| | TTTAATGCCTTTGTATCATGCTATTGCTTCCCGTATGGCT |
| | TTCATTTTCTCCTCCTTGTATAAATCCTGGTTGCTGTCTC |
| | TTTATGAGGAGTTGTGGCCCGTTGTCAGGCAACGTGGCGT |
| | GGTGTGCACTGTGTTTGCTGACGCAACCCCCACTGGTTGG |
| | GGCATTGCCACCACCTGTCAGCTCCTTTCCGGGACTTTCG |
| | CTTTCCCCCTCCCTATTGCCACGGCGGAACTCATCGCCGC |
| | CTGCCTTGCCCGCTGCTGGACAGGGGCTCGGCTGTTGGGC |
| | ACTGACAATTCCGTGGTGTTGTCGGGGAAATCATCGTCCT |
| | TTCCTTGGCTGCTCGCCTGTGTTGCCACCTGGATTCTGCG |
| | CGGGACGTCCTTCTGCTACGTCCCTTCGGCCCTCAATCCA |
| | GCGGACCTTCCTTCCCGCGGCCTGCTGCCGGCTCTGCGGC |
| | CTCTTCCGCGTCTTCGAGATCTGCCTCGACTGTGCCTTCT |
| | AGTTGCCAGCCATCTGTTGTTTGCCCCTCCCCCGTGCCTT |
| | CCTTGACCCTGGAAGGTGCCACTCCCACTGTCCTTTCCTA |
| | ATAAAATGAGGAAATTGCATCGCATTGTCTGAGTAGGTGT |
| | CATTCTATTCTGGGGGGTGGGGTGGGGCAGGACAGCAAGG |
| | GGGAGGATTGGGAAGACAATAGCAGGCATGCTGGGGACTC |
| | GAGTTAAGGGCGAATTCCCGATTAGGATCTTCCTAGAGCA |
| | TGGCTACGTAGATAAGTAGCATGGCGGGTTAATCATTAAC |
| | TACAAGGAACCCCTAGTGATGGAGTTGGCCACTCCCTCTC |
| | TGCGCGCTCGCTCGCTCACTGAGGCCGGGCGACCAAAGGT |
| | CGCCCGACGCCCGGGCTTTGCCCGGGCGGCCTCAGTGAGC |
| | GAGCGAGCGCGCAG (SEQ ID NO: 67) |
| pHZ56 (human DWORF | CTGCGCGCTCGCTCGCTCACTGAGGCCGCCCGGGCAAAGC |
| version of pHZ20) | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGGTCATGGAGAAGACCCACCTTGCAGATGTCCTCAC |
| | TGGGGCTGGCAGAGCCGGCAACCTGCCTAAGGCTGCTCAG |
| | TCCATTAGGAGCCAGTAGCCTGGAAGATGTCTTTACCCCC |
| | AGCATCAGTTCAAGTGGAGCAGCACATAACTCTTGCCCTC |
| | TGCCTTCCAAGATTCTGGTGCTGAGACTTATGGAGTGTCT |
| | TGGAGGTTGCCTTCTGCCCCCCAACCCTGCTCCCAGCTGG |
| | CCCTCCCAGGCCTGGGTTGCTGGCCTCTGCTTTATCAGGA |
| | TTCTCAAGAGGGACAGCTGGTTTATGTTGCATGACTGTTC |
| | CCTGCATATCTGCTCTGGTTTTAAATAGCTTATCTGAGCA |
| | GCTGGAGGACCACATGGGCTTATATGGCGTGGGGTACATG |
| | TTCCTGTAGCCTTGTCCCTGGCACCTGCCAAAATAGCAGC |
| | CAACACCCCCCACCCCCACCGCCATCCCCCTGCCCCACCC |
| | GTCCCCTGTCGCACATTCCTCCCTCCGCAGGGCTGGCTCA |
| | CCAGGCCCCAGCCCACATGCCTGCTTAAAGCCCTCTCCAT |
| | CCTCTGCCTCACCCAGTCCCCGCTGAGACTGAGCAGACGC |
| | CTCCATAACTGGTAAGTACCGCCTATAGACTCTATAGGCA |
| | CACCCCTTTGGCTCTTATGCATGCTGACAGACTAACAGAC |
| | TGTTCCTTTCCTGGGTCTTTTCTGCAGGCCTGTACGGAAG |
| | TGTTACTTCTGCTCTAAAAGCTGCGGAATTGTACCCGCGG |
| | CCGATCCAATCGATACAGATCTAGCGGCCGCCCGCCACCA |
| | TGGCTGAAAAAGCGGGGTCTACATTTTCACACCTTCTGGT |
| | TCCTATTCTTCTCCTGATTGGCTGGATTGTGGGCTGCATC |
| | ATAATGATTTATGTTGTCTTCTCTTAGAAGCTTTGGATCC |
| | AATCAACCTCTGGATTACAAAATTTGTGAAAGATTGACTG |
| | GTATTCTTAACTATGTTGCTCCTTTTACGCTATGTGGATA |
| | CGCTGCTTTAATGCCTTTGTATCATGCTATTGCTTCCCGT |
| | ATGGCTTTCATTTTCTCCTCCTTGTATAAATCCTGGTTGC |
| | TGTCTCTTTATGAGGAGTTGTGGCCCGTTGTCAGGCAACG |
| | TGGCGTGGTGTGCACTGTGTTTGCTGACGCAACCCCCACT |
| | GGTTGGGGCATTGCCACCACCTGTCAGCTCCTTTCCGGGA |
| | CTTTCGCTTTCCCCCTCCCTATTGCCACGGCGGAACTCAT |
| | CGCCGCCTGCCTTGCCCGCTGCTGGACAGGGGCTCGGCTG |
| | TTGGGCACTGACAATTCCGTGGTGTTGTCGGGGAAATCAT |
| | CGTCCTTTCCTTGGCTGCTCGCCTGTGTTGCCACCTGGAT |
| | TCTGCGCGGGACGTCCTTCTGCTACGTCCCTTCGGCCCTC |
| | AATCCAGCGGACCTTCCTTCCCGCGGCCTGCTGCCGGCTC |
| | TGCGGCCTCTTCCGCGTCTTCGAGATCTGCCTCGACTGTG |
| | CCTTCTAGTTGCCAGCCATCTGTTGTTTGCCCCTCCCCCG |
| | TGCCTTCCTTGACCCTGGAAGGTGCCACTCCCACTGTCCT |
| | TTCCTAATAAAATGAGGAAATTGCATCGCATTGTCTGAGT |
| | AGGTGTCATTCTATTCTGGGGGGTGGGGTGGGGCAGGACA |
| | GCAAGGGGGAGGATTGGGAAGACAATAGCAGGCATGCTGG |
| | GGACTCGAGTTAAGGGCGAATTCCCGATTAGGATCTTCCT |
| | AGAGCATGGCTACGTAGATAAGTAGCATGGCGGGTTAATC |
| | ATTAACTACAAGGAACCCCTAGTGATGGAGTTGGCCACTC |
| | CCTCTCTGCGCGCTCGCTCGCTCACTGAGGCCGGGCGACC |
| | AAAGGTCGCCCGACGCCCGGGCTTTGCCCGGGCGGCCTCA |
| | GTGAGCGAGCGAGCGCGCAG (SEQ ID NO: 68) |
| pHZ57 (human DWORF | CTGCGCGCTCGCTCGCTCACTGAGGCCGCCCGGGCAAAGC |
| version of pHZ21) | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGAACTGGCCTGCCCGAGACCAAACGTGCGGAACGTA |
| | GTTAAGTGTTAGAGGTAGGATTTGAAGCCTGTCGATCATT |
| | CTGATTCTCCTTTTCTCTACGTCTGCTTCCTGTCAATGGG |
| | CATCCTCACTGTCAAATGCAGATGGTACAGCAGGGCTTGG |
| | TCTCAGCCAGGCAGGCCTCTCCCCAGTCTCCATGGCTCAG |
| | CTGTCCAGCAGTTTCATCCCTAGACCATCCCAAACATGGT |
| | TGAGAAGCTCTGAGGGGAGGACCCAGCACTGCCCGGCCCC |
| | TGAAGATAATCAGCAGTCCTGCTCAGCATATCAATCCAAG |
| | CCCACTCTAGACAGAGATGCCGGTGCCCAGTTTTCTATTT |
| | TTAACTGGTGTGAACTGAAGGAAAAGCACAGCATTAGAAG |
| | TCCAAGCACCTTCAGATTAAAAATAACTAAGGTAAGGGCC |
| | ATGTGGGTAGGGGAGGTGGTGTGAGACGGTCCTGTCTCTC |
| | CTCTATCTGCCCATCGGCCCTTTGGGGAGGAGGAATGTGC |
| | CCAAGGACTAAAAAAAGGCCCTGGAGCCAGAGGGGCGAGG |
| | GCAGCAGACCTTTCATGGGCAAACCTCAGGGCTGCTGTCG |
| | TCATGGAGAAGACCCACCTTGCAGATGTCCTCACTGGGGC |
| | TGGCAGAGCCGGCAACCTGCCTAAGGCTGCTCAGTCCATT |
| | AGGAGCCAGTAGCCTGGAAGATGTCTTTACCCCCAGCATC |
| | AGTTCAAGTGGAGCAGCACATAACTCTTGCCCTCTGCCTT |
| | CCAAGATTCTGGTGCTGAGACTTATGGAGTGTCTTGGAGG |
| | TTGCCTTCTGCCCCCCAACCCTGCTCCCAGCTGGCCCTCC |
| | CAGGCCTGGGTTGCTGGCCTCTGCTTTATCAGGATTCTCA |
| | AGAGGGACAGCTGGTTTATGTTGCATGACTGTTCCCTGCA |
| | TATCTGCTCTGGTTTTAAATAGCTTATCTGAGCAGCTGGA |
| | GGACCACATGGGCTTATATGGCGTGGGGTACATGTTCCTG |
| | TAGCCTTGTCCCTGGCACCTGCCAAAATAGCAGCCAACAC |
| | CCCCCACCCCCACCGCCATCCCCCTGCCCCACCCGTCCCC |
| | TGTCGCACATTCCTCCCTCCGCAGGGCTGGCTCACCAGGC |
| | CCCAGCCCACATGCCTGCTTAAAGCCCTCTCCATCCTCTG |
| | CCTCACCCAGTCCCCGCTGAGACTGAGCAGACGCCTCCAT |
| | AACTGGTAAGTACCGCCTATAGACTCTATAGGCACACCCC |
| | TTTGGCTCTTATGCATGCTGACAGACTAACAGACTGTTCC |
| | TTTCCTGGGTCTTTTCTGCAGGCCTGTACGGAAGTGTTAC |
| | TTCTGCTCTAAAAGCTGCGGAATTGTACCCGCGGCCGATC |
| | CAATCGATACAGATCTAGCGGCCGCCCGCCACCATGGCTG |
| | AAAAAGCGGGGTCTACATTTTCACACCTTCTGGTTCCTAT |
| | TCTTCTCCTGATTGGCTGGATTGTGGGCTGCATCATAATG |
| | ATTTATGTTGTCTTCTCTTAGAAGCTTTGGATCCAATCAA |
| | CCTCTGGATTACAAAATTTGTGAAAGATTGACTGGTATTC |
| | TTAACTATGTTGCTCCTTTTACGCTATGTGGATACGCTGC |
| | TTTAATGCCTTTGTATCATGCTATTGCTTCCCGTATGGCT |
| | TTCATTTTCTCCTCCTTGTATAAATCCTGGTTGCTGTCTC |
| | TTTATGAGGAGTTGTGGCCCGTTGTCAGGCAACGTGGCGT |
| | GGTGTGCACTGTGTTTGCTGACGCAACCCCCACTGGTTGG |
| | GGCATTGCCACCACCTGTCAGCTCCTTTCCGGGACTTTCG |
| | CTTTCCCCCTCCCTATTGCCACGGCGGAACTCATCGCCGC |
| | CTGCCTTGCCCGCTGCTGGACAGGGGCTCGGCTGTTGGGC |
| | ACTGACAATTCCGTGGTGTTGTCGGGGAAATCATCGTCCT |
| | TTCCTTGGCTGCTCGCCTGTGTTGCCACCTGGATTCTGCG |
| | CGGGACGTCCTTCTGCTACGTCCCTTCGGCCCTCAATCCA |
| | GCGGACCTTCCTTCCCGCGGCCTGCTGCCGGCTCTGCGGC |
| | CTCTTCCGCGTCTTCGAGATCTGCCTCGACTGTGCCTTCT |
| | AGTTGCCAGCCATCTGTTGTTTGCCCCTCCCCCGTGCCTT |
| | CCTTGACCCTGGAAGGTGCCACTCCCACTGTCCTTTCCTA |
| | ATAAAATGAGGAAATTGCATCGCATTGTCTGAGTAGGTGT |
| | CATTCTATTCTGGGGGGTGGGGTGGGGCAGGACAGCAAGG |
| | GGGAGGATTGGGAAGACAATAGCAGGCATGCTGGGGACTC |
| | GAGTTAAGGGCGAATTCCCGATTAGGATCTTCCTAGAGCA |
| | TGGCTACGTAGATAAGTAGCATGGCGGGTTAATCATTAAC |
| | TACAAGGAACCCCTAGTGATGGAGTTGGCCACTCCCTCTC |
| | TGCGCGCTCGCTCGCTCACTGAGGCCGGGCGACCAAAGGT |
| | CGCCCGACGCCCGGGCTTTGCCCGGGCGGCCTCAGTGAGC |
| | GAGCGAGCGCGCAG (SEQ ID NO: 69) |
| pHZ58 (human DWORF | CTGCGCGCTCGCTCGCTCACTGAGGCCGCCCGGGCAAAGC |
| version of pHZ22) | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGAACTGGCCTGCCCGAGACCAAACGTGCGGAACGTA |
| | GTTAAGTGTTAGAGGTAGGATTTGAAGCCTGTCGATCATT |
| | CTGATTCTCCTTTTCTCTACGTCTGCTTCCTGTCAATGGG |
| | CATCCTCACTGTCAAATGCAGATGGTACAGCAGGGCTTGG |
| | TCTCAGCCAGGCAGGCCTCTCCCCAGTCTCCATGGCTCAG |
| | CTGTCCAGCAGTTTCATCCCTAGACCATCCCAAACATGGT |
| | TGAGAAGCTCTGAGGGGAGGACCCAGCACTGCCCGGCCCC |
| | TGAAGATAATCAGCAGTCCTGCTCAGCATATCAATCCAAG |
| | CCCACTCTAGACAGAGATGCCGGTGCCCAGTTTTCTATTT |
| | TTAACTGGTGTGAACTGAAGGAAAAGCACAGCATTAGAAG |
| | TCCAAGCACCTTCAGATTAAAAATAACTAAGGTAAGGGCC |
| | ATGTGGGTAGGGGAGGTGGTGTGAGACGGTCCTGTCTCTC |
| | CTCTATCTGCCCATCGGCCCTTTGGGGAGGAGGAATGTGC |
| | CCAAGGACTAAAAAAAGGCCCTGGAGCCAGAGGGGCGAGG |
| | GCAGCAGACCTTTCATGGGCAAACCTCAGGGCTGCTGTCG |
| | TCATGGAGAAGACCCACCTTGCAGATGTCCTCACTGGGGC |
| | TGGCAGAGCCGGCAACCTGCCTAAGGCTGCTCAGTCCATT |
| | AGGAGCCAGTAGCCTGGAAGATGTCTTTACCCCCAGCATC |
| | AGTTCAAGTGGAGCAGCACATAACTCTTGCCCTCTGCCTT |
| | CCAAGATTCTGGTGCTGAGACTTATGGAGTGTCTTGGAGG |
| | TTGCCTTCTGCCCCCCAACCCTGCTCCCAGCTGGCCCTCC |
| | CAGGCCTGGGTTGCTGGCCTCTGCTTTATCAGGATTCTCA |
| | AGAGGGACAGCTGGTTTATGTTGCATGACTGTTCCCTGCA |
| | TATCTGCTCTGGTTTTAAATAGCTTATCTGAGCAGCTGGA |
| | GGACCACATGGGCTTATATGGCGTGGGGTACATGTTCCTG |
| | TAGCCTTGTCCCTGGCACCTGCCAAAATAGCAGCCAACAC |
| | CCCCCACCCCCACCGCCATCCCCCTGCCCCACCCGTCCCC |
| | TGTCGCACATTCCTCCCTCCGCAGGGCTGGCTCACCAGGC |
| | CCCAGCCCACATGCCTGCTTAAAGCCCTCTCCATCCTCTG |
| | CCTCACCCAGTCCCCGCTGAGACTGAGCAGACGCCTCCAT |
| | AACTGGTAAGTACCGCCTATAGACTCTATAGGCACACCCC |
| | TTTGGCTCTTATGCATGCTGACAGACTAACAGACTGTTCC |
| | TTTCCTGGGTCTTTTCTGCAGGCCTGTACGGAAGTGTTAC |
| | TTCTGCTCTAAAAGCTGCGGAATTGTACCCGCGGCCGATC |
| | CAATCGATACAGATCTAGCGGCCGCCCGCCACCATGGCTG |
| | AAAAAGCGGGGTCTACATTTTCACACCTTCTGGTTCCTAT |
| | TCTTCTCCTGATTGGCTGGATTGTGGGCTGCATCATAATG |
| | ATTTATGTTGTCTTCTCTTAGAAGCTTTGGATCCAATCAA |
| | CCTCTGGATTACAAAATTTGTGAAAGATTGACTGGTATTC |
| | TTAACTATGTTGCTCCTTTTACGCTATGTGGATACGCTGC |
| | TTTAATGCCTTTGTATCATGCTATTGCTTCCCGTATGGCT |
| | TTCATTTTCTCCTCCTTGTATAAATCCTGGTTGCTGTCTC |
| | TTTATGAGGAGTTGTGGCCCGTTGTCAGGCAACGTGGCGT |
| | GGTGTGCACTGTGTTTGCTGACGCAACCCCCACTGGTTGG |
| | GGCATTGCCACCACCTGTCAGCTCCTTTCCGGGACTTTCG |
| | CTTTCCCCCTCCCTATTGCCACGGCGGAACTCATCGCCGC |
| | CTGCCTTGCCCGCTGCTGGACAGGGGCTCGGCTGTTGGGC |
| | ACTGACAATTCCGTGGTGTTGTCGGGGAAATCATCGTCCT |
| | TTCCTTGGCTGCTCGCCTGTGTTGCCACCTGGATTCTGCG |
| | CGGGACGTCCTTCTGCTACGTCCCTTCGGCCCTCAATCCA |
| | GCGGACCTTCCTTCCCGCGGCCTGCTGCCGGCTCTGCGGC |
| | CTCTTCCGCGTCTTCGAGATCTGCCTCGACTGTGCCTTCT |
| | AGTTGCCAGCCATCTGTTGTTTGCCCCTCCCCCGTGCCTT |
| | CCTTGACCCTGGAAGGTGCCACTCCCACTGTCCTTTCCTA |
| | ATAAAATGAGGAAATTGCATCGCATTGTCTGAGTAGGTGT |
| | CATTCTATTCTGGGGGGTGGGGTGGGGCAGGACAGCAAGG |
| | GGGAGGATTGGGAAGACAATAGCAGGCATGCTGGGGACTC |
| | GAGTTAAGGGCGAATTCCCGATTAGGATCTTCCTAGAGCA |
| | TGGCTACGTAGATAAGTAGCATGGCGGGTTAATCATTAAC |
| | TACAAGGAACCCCTAGTGATGGAGTTGGCCACTCCCTCTC |
| | TGCGCGCTCGCTCGCTCACTGAGGCCGGGCGACCAAAGGT |
| | CGCCCGACGCCCGGGCTTTGCCCGGGCGGCCTCAGTGAGC |
| | GAGCGAGCGCGCAG (SEQ ID NO: 70) |
| pHZ59 (human DWORF | CTGCGCGCTCGCTCGCTCACTGAGGCCGCCCGGGCAAAGC |
| version of pHZ23) | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGCCTTCAGATTAAAAATAACTAAGGTAAGGGCCATG |
| | TGGGTAGGGGAGGTGGTGTGAGACGGTCCTGTCTCTCCTC |
| | TATCTGCCCATCGGCCCTTTGGGGAGGAGGAATGTGCCCA |
| | AGGACTAAAAAAAGGCCCTGGAGCCAGAGGGGCGAGGGCA |
| | GCAGACCTTTCATGGGCAAACCTCAGGGCTGCTGTCGTCA |
| | TGGAGAAGACCCACCTTGCAGATGTCCTCACTGGGGCTGG |
| | CAGAGCCGGCAACCTGCCTAAGGCTGCTCAGTCCATTAGG |
| | AGCCAGTAGCCTGGAAGATGTCTTTACCCCCAGCATCAGT |
| | TCAAGTGGAGCAGCACATAACTCTTGCCCTCTGCCTTCCA |
| | AGATTCTGGTGCTGAGACTTATGGAGTGTCTTGGAGGTTG |
| | CCTTCTGCCCCCCAACCCTGCTCCCAGCTGGCCCTCCCAG |
| | GCCTGGGTTGCTGGCCTCTGCTTTATCAGGATTCTCAAGA |
| | GGGACAGCTGGTTTATGTTGCATGACTGTTCCCTGCATAT |
| | CTGCTCTGGTTTTAAATAGCTTATCTGAGCAGCTGGAGGA |
| | CCACATGGGCTTATATGGCGTGGGGTACATGTTCCTGTAG |
| | CCTTGTCCCTGGCACCTGCCAAAATAGCAGCCAACACCCC |
| | CCACCCCCACCGCCATCCCCCTGCCCCACCCGTCCCCTGT |
| | CGCACATTCCTCCCTCCGCAGGGCTGGCTCACCAGGCCCC |
| | AGCCCACATGCCTGCTTAAAGCCCTCTCCATCCTCTGCCT |
| | CACCCAGTCCCCGCTGAGACTGAGCAGACGCCTCCATAAC |
| | TGGTAAGTACCGCCTATAGACTCTATAGGCACACCCCTTT |
| | GGCTCTTATGCATGCTGACAGACTAACAGACTGTTCCTTT |
| | CCTGGGTCTTTTCTGCAGGCCTGTACGGAAGTGTTACTTC |
| | TGCTCTAAAAGCTGCGGAATTGTACCCGCGGCCGATCCAA |
| | TCGATACAGATCTAGCGGCCGCCCGCCACCATGGCTGAAA |
| | AAGCGGGGTCTACATTTTCACACCTTCTGGTTCCTATTCT |
| | TCTCCTGATTGGCTGGATTGTGGGCTGCATCATAATGATT |
| | TATGTTGTCTTCTCTTAGAAGCTTTGGATCCAATCAACCT |
| | CTGGATTACAAAATTTGTGAAAGATTGACTGGTATTCTTA |
| | ACTATGTTGCTCCTTTTACGCTATGTGGATACGCTGCTTT |
| | AATGCCTTTGTATCATGCTATTGCTTCCCGTATGGCTTTC |
| | ATTTTCTCCTCCTTGTATAAATCCTGGTTGCTGTCTCTTT |
| | ATGAGGAGTTGTGGCCCGTTGTCAGGCAACGTGGCGTGGT |
| | GTGCACTGTGTTTGCTGACGCAACCCCCACTGGTTGGGGC |
| | ATTGCCACCACCTGTCAGCTCCTTTCCGGGACTTTCGCTT |
| | TCCCCCTCCCTATTGCCACGGCGGAACTCATCGCCGCCTG |
| | CCTTGCCCGCTGCTGGACAGGGGCTCGGCTGTTGGGCACT |
| | GACAATTCCGTGGTGTTGTCGGGGAAATCATCGTCCTTTC |
| | CTTGGCTGCTCGCCTGTGTTGCCACCTGGATTCTGCGCGG |
| | GACGTCCTTCTGCTACGTCCCTTCGGCCCTCAATCCAGCG |
| | GACCTTCCTTCCCGCGGCCTGCTGCCGGCTCTGCGGCCTC |
| | TTCCGCGTCTTCGAGATCTGCCTCGACTGTGCCTTCTAGT |
| | TGCCAGCCATCTGTTGTTTGCCCCTCCCCCGTGCCTTCCT |
| | TGACCCTGGAAGGTGCCACTCCCACTGTCCTTTCCTAATA |
| | AAATGAGGAAATTGCATCGCATTGTCTGAGTAGGTGTCAT |
| | TCTATTCTGGGGGGTGGGGTGGGGCAGGACAGCAAGGGGG |
| | AGGATTGGGAAGACAATAGCAGGCATGCTGGGGACTCGAG |
| | TTAAGGGCGAATTCCCGATTAGGATCTTCCTAGAGCATGG |
| | CTACGTAGATAAGTAGCATGGCGGGTTAATCATTAACTAC |
| | AAGGAACCCCTAGTGATGGAGTTGGCCACTCCCTCTCTGC |
| | GCGCTCGCTCGCTCACTGAGGCCGGGCGACCAAAGGTCGC |
| | CCGACGCCCGGGCTTTGCCCGGGCGGCCTCAGTGAGCGAG |
| | CGAGCGCGCAG (SEQ ID NO: 71) |
| pHZ60 (human DWORF | CTGCGCGCTCGCTCGCTCACTGAGGCCGCCCGGGCAAAGC |
| version of pHZ24) | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGGTCATGGAGAAGACCCACCTTGCAGATGTCCTCAC |
| | TGGGGCTGGCAGAGCCGGCAACCTGCCTAAGGCTGCTCAG |
| | TCCATTAGGAGCCAGTAGCCTGGAAGATGTCTTTACCCCC |
| | AGCATCAGTTCAAGTGGAGCAGCACATAACTCTTGCCCTC |
| | TGCCTTCCAAGATTCTGGTGCTGAGACTTATGGAGTGTCT |
| | TGGAGGTTGCCTTCTGCCCCCCAACCCTGCTCCCAGCTGG |
| | CCCTCCCAGGCCTGGGTTGCTGGCCTCTGCTTTATCAGGA |
| | TTCTCAAGAGGGACAGCTGGTTTATGTTGCATGACTGTTC |
| | CCTGCATATCTGCTCTGGTTTTAAATAGCTTATCTGAGCA |
| | GCTGGAGGACCACATGGGCTTATATGGCGTGGGGTACATG |
| | TTCCTGTAGCCTTGTCCCTGGCACCTGCCAAAATAGCAGC |
| | CAACACCCCCCACCCCCACCGCCATCCCCCTGCCCCACCC |
| | GTCCCCTGTCGCACATTCCTCCCTCCGCAGGGCTGGCTCA |
| | CCAGGCCCCAGCCCACATGCCTGCTTAAAGCCCTCTCCAT |
| | CCTCTGCCTCACCCAGTCCCCGCTGAGACTGAGCAGACGC |
| | CTCCAGCGGCCGCCCGCCACCATGGCCGAGAAGGCCGGAT |
| | CTACCTTCAGCCACCTGCTGGTCCCTATTCTGCTGCTGAT |
| | CGGCTGGATCGTGGGCTGCATCATCATGATCTACGTGGTG |
| | TTCAGCTGAAAGCTTTGGATCCAATCAACCTCTGGATTAC |
| | AAAATTTGTGAAAGATTGACTGGTATTCTTAACTATGTTG |
| | CTCCTTTTACGCTATGTGGATACGCTGCTTTAATGCCTTT |
| | GTATCATGCTATTGCTTCCCGTATGGCTTTCATTTTCTCC |
| | TCCTTGTATAAATCCTGGTTGCTGTCTCTTTATGAGGAGT |
| | TGTGGCCCGTTGTCAGGCAACGTGGCGTGGTGTGCACTGT |
| | GTTTGCTGACGCAACCCCCACTGGTTGGGGCATTGCCACC |
| | ACCTGTCAGCTCCTTTCCGGGACTTTCGCTTTCCCCCTCC |
| | CTATTGCCACGGCGGAACTCATCGCCGCCTGCCTTGCCCG |
| | CTGCTGGACAGGGGCTCGGCTGTTGGGCACTGACAATTCC |
| | GTGGTGTTGTCGGGGAAATCATCGTCCTTTCCTTGGCTGC |
| | TCGCCTGTGTTGCCACCTGGATTCTGCGCGGGACGTCCTT |
| | CTGCTACGTCCCTTCGGCCCTCAATCCAGCGGACCTTCCT |
| | TCCCGCGGCCTGCTGCCGGCTCTGCGGCCTCTTCCGCGTC |
| | TTCGAGATCTGCCTCGACTGTGCCTTCTAGTTGCCAGCCA |
| | TCTGTTGTTTGCCCCTCCCCCGTGCCTTCCTTGACCCTGG |
| | AAGGTGCCACTCCCACTGTCCTTTCCTAATAAAATGAGGA |
| | AATTGCATCGCATTGTCTGAGTAGGTGTCATTCTATTCTG |
| | GGGGGTGGGGTGGGGCAGGACAGCAAGGGGGAGGATTGGG |
| | AAGACAATAGCAGGCATGCTGGGGACTCGAGTTAAGGGCG |
| | AATTCCCGATTAGGATCTTCCTAGAGCATGGCTACGTAGA |
| | TAAGTAGCATGGCGGGTTAATCATTAACTACAAGGAACCC |
| | CTAGTGATGGAGTTGGCCACTCCCTCTCTGCGCGCTCGCT |
| | CGCTCACTGAGGCCGGGCGACCAAAGGTCGCCCGACGCCC |
| | GGGCTTTGCCCGGGCGGCCTCAGTGAGCGAGCGAGCGCGC |
| | AG (SEQ ID NO: 72) |
| pHZ61 (human DWORF | CTGCGCGCTCGCTCGCTCACTGAGGCCGCCCGGGCAAAGC |
| version of pHZ25) | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGCCTTCAGATTAAAAATAACTAAGGTAAGGGCCATG |
| | TGGGTAGGGGAGGTGGTGTGAGACGGTCCTGTCTCTCCTC |
| | TATCTGCCCATCGGCCCTTTGGGGAGGAGGAATGTGCCCA |
| | AGGACTAAAAAAAGGCCCTGGAGCCAGAGGGGCGAGGGCA |
| | GCAGACCTTTCATGGGCAAACCTCAGGGCTGCTGTCGTCA |
| | TGGAGAAGACCCACCTTGCAGATGTCCTCACTGGGGCTGG |
| | CAGAGCCGGCAACCTGCCTAAGGCTGCTCAGTCCATTAGG |
| | AGCCAGTAGCCTGGAAGATGTCTTTACCCCCAGCATCAGT |
| | TCAAGTGGAGCAGCACATAACTCTTGCCCTCTGCCTTCCA |
| | AGATTCTGGTGCTGAGACTTATGGAGTGTCTTGGAGGTTG |
| | CCTTCTGCCCCCCAACCCTGCTCCCAGCTGGCCCTCCCAG |
| | GCCTGGGTTGCTGGCCTCTGCTTTATCAGGATTCTCAAGA |
| | GGGACAGCTGGTTTATGTTGCATGACTGTTCCCTGCATAT |
| | CTGCTCTGGTTTTAAATAGCTTATCTGAGCAGCTGGAGGA |
| | CCACATGGGCTTATATGGCGTGGGGTACATGTTCCTGTAG |
| | CCTTGTCCCTGGCACCTGCCAAAATAGCAGCCAACACCCC |
| | CCACCCCCACCGCCATCCCCCTGCCCCACCCGTCCCCTGT |
| | CGCACATTCCTCCCTCCGCAGGGCTGGCTCACCAGGCCCC |
| | AGCCCACATGCCTGCTTAAAGCCCTCTCCATCCTCTGCCT |
| | CACCCAGTCCCCGCTGAGACTGAGCAGACGCCTCCATAAC |
| | TGGTAAGTACCGCCTATAGACTCTATAGGCACACCCCTTT |
| | GGCTCTTATGCATGCTGACAGACTAACAGACTGTTCCTTT |
| | CCTGGGTCTTTTCTGCAGGCCTGTACGGAAGTGTTACTTC |
| | TGCTCTAAAAGCTGCGGAATTGTACCCGCGGCCGATCCAA |
| | TCGATACAGATCTAGCGGCCGCCCGCCACCATGGCTGAAA |
| | AAGCGGGGTCTACATTTTCACACCTTCTGGTTCCTATTCT |
| | TCTCCTGATTGGCTGGATTGTGGGCTGCATCATAATGATT |
| | TATGTTGTCTTCTCTTAGAAGCTTTGGATCCAATCAACCT |
| | CTGGATTACAAAATTTGTGAAAGATTGACTGGTATTCTTA |
| | ACTATGTTGCTCCTTTTACGCTATGTGGATACGCTGCTTT |
| | AATGCCTTTGTATCATGCTATTGCTTCCCGTATGGCTTTC |
| | ATTTTCTCCTCCTTGTATAAATCCTGGTTGCTGTCTCTTT |
| | ATGAGGAGTTGTGGCCCGTTGTCAGGCAACGTGGCGTGGT |
| | GTGCACTGTGTTTGCTGACGCAACCCCCACTGGTTGGGGC |
| | ATTGCCACCACCTGTCAGCTCCTTTCCGGGACTTTCGCTT |
| | TCCCCCTCCCTATTGCCACGGCGGAACTCATCGCCGCCTG |
| | CCTTGCCCGCTGCTGGACAGGGGCTCGGCTGTTGGGCACT |
| | GACAATTCCGTGGTGTTGTCGGGGAAATCATCGTCCTTTC |
| | CTTGGCTGCTCGCCTGTGTTGCCACCTGGATTCTGCGCGG |
| | GACGTCCTTCTGCTACGTCCCTTCGGCCCTCAATCCAGCG |
| | GACCTTCCTTCCCGCGGCCTGCTGCCGGCTCTGCGGCCTC |
| | TTCCGCGTCTTCGAGATCTGCCTCGACTGTGCCTTCTAGT |
| | TGCCAGCCATCTGTTGTTTGCCCCTCCCCCGTGCCTTCCT |
| | TGACCCTGGAAGGTGCCACTCCCACTGTCCTTTCCTAATA |
| | AAATGAGGAAATTGCATCGCATTGTCTGAGTAGGTGTCAT |
| | TCTATTCTGGGGGGTGGGGTGGGGCAGGACAGCAAGGGGG |
| | AGGATTGGGAAGACAATAGCAGGCATGCTGGGGACTCGAG |
| | TTAAGGGCAGCCAGAAGTCAGATGCTCAAGGGGCTTCATG |
| | ATGTCCCCATAATTTTTGGCAGAGGGAAAAAGATCGGATC |
| | CTCAGGCGTAGTTCACCCCGTCCTCGAGGCCGCCCGGGTC |
| | GACTAAAAAACCTCCCACACCTCCCCCTGAACCTGAAACA |
| | TAAAATGAATGCAATTGTTGTTGTTAACTTGTTTATTGCA |
| | GCTTATAATGGTTACAAATAAAGCAATAGCATCACAAATT |
| | TCACAAATAAAGCATTTTTTTCACTGCATTCTAGTTGTGG |
| | TTTGTCCAAACTCATCAATGTATCTTATCATGTCTGGATC |
| | CGCGCGGCCGTCAGCTGAACACCACGTAGATCATGATGAT |
| | GCAGCCCACGATCCAGCCGATCAGCAGCAGAATAGGGACC |
| | AGCAGGTGGCTGAAGGTAGATCCGGCCTTCTCGGCCATGG |
| | TGGCGGCTAGCCTATAGTGAGTCGTATTAAGTACTCTAGC |
| | CTTAAGAGCTGTAATTGAACTGGGAGTGGACACCTGTGGA |
| | GAGAAAGGCAAAGTGGATGTCAGTAAGACCAATAGGTGCC |
| | TATCAGAAACGCAAGAGTCTTCTCTGTCTCGACAAGCCCA |
| | GTTTCTATTGGTCTCCTTAAACCTGTCTTGTAACCTTGAT |
| | ACTTACCTGCCCAGTGCCTCACGACCAACTTCTGCAGCTT |
| | AAGTTCGAGACTGTTGTGTCAGAAGCACTGACTGCGTTAG |
| | CAATTTAACTGTGATAAACTACCGCAATAAAGCTCTAGAG |
| | CTTCGGGGATCGTCCCACGGAGCGGTGGGTGCCGGCGGCT |
| | GTCTGGGAAGGGCTCCTTGGGGGGCAGAGGCTTTAAGGTC |
| | CCCCCGGCGCCCACCCCGGGGGGGGCAGAGCCAGCAGGAA |
| | TGTGCCCGGCGCCCAGAGAGGAATGCAACACTTGTGAGCT |
| | GCTATTTTGGCAGCAGCGGCCCCGGCCCCCTCCGTGCTCC |
| | CCCTTCCCCCACAGGAGCCCATATAAGCCCAAGCTATTGT |
| | GTGGCCTCAGAGTTTTGCTATTTTAAACCCGTCGGACGGA |
| | GATACGTGAGTGCCCGAGGGGCTGACACAAGCCAGCCAGC |
| | TGTCACCTCCCAGGGCTGGGGACGCTGATAAGGCAGCGCT |
| | TCGGACCCGACCCTCTGCCGCAGCCCCAGATGCTGTCATG |
| | TGAAAGCCCAGACTGCTTTTATCCCTGCTTGGACTTCTAA |
| | TGCTGTGCTTTTCCTTCAGTTCACACCAGTTAAAAATAGA |
| | AAACTGGGCACCGGCATCTCTGTCTAGAGTGGGCTTGGAT |
| | TGATATGCTGAGCAGGACTGCTGATTATCTTCAGGGGCCG |
| | GGCAGTGCTGGGTCCTCCCCTCAGAGCTTCTCAACCATGT |
| | TTGGGATGGTCTAGGGATGAAACTGCTGGACAGCTGAGCC |
| | ATGGAGACTGGGGAGAGGCCTGCCTGGCTGAGACCAAGCC |
| | CTGCTGTACCATCTGCATTTGACAGTGAGGATGCCCATTG |
| | ACAGGAAGCAGACGTAGAGAAAAGGAGAATCAGAATGATC |
| | GACAGGCTTCAAATCCTACCTCTAACACTTAACTACGTTC |
| | CGCACGTTTGGTCTCGGGCAGGCCAGTTGAATTCCCGATT |
| | AGGATCTTCCTAGAGCATGGCTACGTAGATAAGTAGCATG |
| | GCGGGTTAATCATTAACTACAAGGAACCCCTAGTGATGGA |
| | GTTGGCCACTCCCTCTCTGCGCGCTCGCTCGCTCACTGAG |
| | GCCGGGCGACCAAAGGTCGCCCGACGCCCGGGCTTTGCCC |
| | GGGCGGCCTCAGTGAGCGAGCGAGCGCGCAG |
| | (SEQ ID NO: 73) |
| pHZ62 (human DWORF | CTGCGCGCTCGCTCGCTCACTGAGGCCGCCCGGGCAAAGC |
| version of pHZ33) | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCGCCC |
| | TTAAGCCTTCAGATTAAAAATAACTAAGGTAAGGGCCATG |
| | TGGGTAGGGGAGGTGGTGTGAGACGGTCCTGTCTCTCCTC |
| | TATCTGCCCATCGGCCCTTTGGGGAGGAGGAATGTGCCCA |
| | AGGACTAAAAAAAGGCCCTGGAGCCAGAGGGGCGAGGGCA |
| | GCAGACCTTTCATGGGCAAACCTCAGGGCTGCTGTCGTCA |
| | TGGAGAAGACCCACCTTGCAGATGTCCTCACTGGGGCTGG |
| | CAGAGCCGGCAACCTGCCTAAGGCTGCTCAGTCCATTAGG |
| | AGCCAGTAGCCTGGAAGATGTCTTTACCCCCAGCATCAGT |
| | TCAAGTGGAGCAGCACATAACTCTTGCCCTCTGCCTTCCA |
| | AGATTCTGGTGCTGAGACTTATGGAGTGTCTTGGAGGTTG |
| | CCTTCTGCCCCCCAACCCTGCTCCCAGCTGGCCCTCCCAG |
| | GCCTGGGTTGCTGGCCTCTGCTTTATCAGGATTCTCAAGA |
| | GGGACAGCTGGTTTATGTTGCATGACTGTTCCCTGCATAT |
| | CTGCTCTGGTTTTAAATAGCTTATCTGAGCAGCTGGAGGA |
| | CCACATGGGCTTATATGGCGTGGGGTACATGTTCCTGTAG |
| | CCTTGTCCCTGGCACCTGCCAAAATAGCAGCCAACACCCC |
| | CCACCCCCACCGCCATCCCCCTGCCCCACCCGTCCCCTGT |
| | CGCACATTCCTCCCTCCGCAGGGCTGGCTCACCAGGCCCC |
| | AGCCCACATGCCTGCTTAAAGCCCTCTCCATCCTCTGCCT |
| | CACCCAGTCCCCGCTGAGACTGAGCAGACGCCTCCATAAC |
| | TGGTAAGTACCGCCTATAGACTCTATAGGCACACCCCTTT |
| | GGCTCTTATGCATGCTGACAGACTAACAGACTGTTCCTTT |
| | CCTGGGTCTTTTCTGCAGGCCTGTACGGAAGTGTTACTTC |
| | TGCTCTAAAAGCTGCGGAATTGTACCCGCGGCCGATCCAA |
| | TCGATACAGATCTAGCGGCCGCCCGCCACCATGGCTGAAA |
| | AAGCGGGGTCTACATTTTCACACCTTCTGGTTCCTATTCT |
| | TCTCCTGATTGGCTGGATTGTGGGCTGCATCATAATGATT |
| | TATGTTGTCTTCTCTTAGAAGCTTTGGATCCAATCAACCT |
| | CTGGATTACAAAATTTGTGAAAGATTGACTGGTATTCTTA |
| | ACTATGTTGCTCCTTTTACGCTATGTGGATACGCTGCTTT |
| | AATGCCTTTGTATCATGCTATTGCTTCCCGTATGGCTTTC |
| | ATTTTCTCCTCCTTGTATAAATCCTGGTTGCTGTCTCTTT |
| | ATGAGGAGTTGTGGCCCGTTGTCAGGCAACGTGGCGTGGT |
| | GTGCACTGTGTTTGCTGACGCAACCCCCACTGGTTGGGGC |
| | ATTGCCACCACCTGTCAGCTCCTTTCCGGGACTTTCGCTT |
| | TCCCCCTCCCTATTGCCACGGCGGAACTCATCGCCGCCTG |
| | CCTTGCCCGCTGCTGGACAGGGGCTCGGCTGTTGGGCACT |
| | GACAATTCCGTGGTGTTGTCGGGGAAATCATCGTCCTTTC |
| | CTTGGCTGCTCGCCTGTGTTGCCACCTGGATTCTGCGCGG |
| | GACGTCCTTCTGCTACGTCCCTTCGGCCCTCAATCCAGCG |
| | GACCTTCCTTCCCGCGGCCTGCTGCCGGCTCTGCGGCCTC |
| | TTCCGCGTCTTCGAGATCTGCCTCGACTGTGCCTTCTAGT |
| | TGCCAGCCATCTGTTGTTTGCCCCTCCCCCGTGCCTTCCT |
| | TGACCCTGGAAGGTGCCACTCCCACTGTCCTTTCCTAATA |
| | AAATGAGGAAATTGCATCGCATTGTCTGAGTAGGTGTCAT |
| | TCTATTCTGGGGGGTGGGGTGGGGCAGGACAGCAAGGGGG |
| | AGGATTGGGAAGACAATAGCAGGCATGCTGGGGACTCGAG |
| | TTAAGGGCAGCCAGAAGTCAGATGCTCAAGGGGCTTCATG |
| | ATGTCCCCATAATTTTTGGCAGAGGGAAAAAGATCGGATC |
| | CTCAGGCGTAGTTCACCCCGTCCTCGAGGCCGCCCGGGTC |
| | GACTAAAAAACCTCCCACACCTCCCCCTGAACCTGAAACA |
| | TAAAATGAATGCAATTGTTGTTGTTAAACTGGCCTGCCCG |
| | AGACCAAACGTGCGGAACGTAGTTAAGTGTTAGAGGTAGG |
| | ATTTGAAGCCTGTCGATCATTCTGATTCTCCTTTTCTCTA |
| | CGTCTGCTTCCTGTCAATGGGCATCCTCACTGTCAAATGC |
| | AGATGGTACAGCAGGGCTTGGTCTCAGCCAGGCAGGCCTC |
| | TCCCCAGTCTCCATGGCTCAGCTGTCCAGCAGTTTCATCC |
| | CTAGACCATCCCAAACATGGTTGAGAAGCTCTGAGGGGAG |
| | GACCCAGCACTGCCCGGCCCCTGAAGATAATCAGCAGTCC |
| | TGCTCAGCATATCAATCCAAGCCCACTCTAGACAGAGATG |
| | CCGGTGCCCAGTTTTCTATTTTTAACTGGTGTGAACTGAA |
| | GGAAAAGCACAGCATTAGAAGTCCAAGCAGGGATAAAAGC |
| | AGTCTGGGCTTTCACATGACAGCATCTGGGGCTGCGGCAG |
| | AGGGTCGGGTCCGAAGCGCTGCCTTATCAGCGTCCCCAGC |
| | CCTGGGAGGTGACAGCTGGCTGGCTTGTGTCAGCCCCTCG |
| | GGCACTCACGTATCTCCGTCCGACGGGTTTAAAATAGCAA |
| | AACTCTGAGGCCACACAATAGCTTGGGCTTATATGGGCTC |
| | CTGTGGGGGAAGGGGGAGCACGGAGGGGGCCGGGGCCGCT |
| | GCTGCCAAAATAGCAGCTCACAAGTGTTGCATTCCTCTCT |
| | GGGCGCCGGGCACATTCCTGCTGGCTCTGCCCGCCCCGGG |
| | GTGGGCGCCGGGGGGACCTTAAAGCCTCTGCCCCCCAAGG |
| | AGCCCTTCCCAGACAGCCGCCGGCACCCACCGCTCCGTGG |
| | GACGATCCCCGAAGCTCTAGAGCTTTATTGCGGTAGTTTA |
| | TCACAGTTAAATTGCTAACGCAGTCAGTGCTTCTGACACA |
| | ACAGTCTCGAACTTAAGCTGCAGAAGTTGGTCGTGAGGCA |
| | CTGGGCAGGTAAGTATCAAGGTTACAAGACAGGTTTAAGG |
| | AGACCAATAGAAACTGGGCTTGTCGAGACAGAGAAGACTC |
| | TTGCGTTTCTGATAGGCACCTATTGGTCTTACTGACATCC |
| | ACTTTGCCTTTCTCTCCACAGGTGTCCACTCCCAGTTCAA |
| | TTACAGCTCTTAAGGCTAGAGTACTTAATACGACTCACTA |
| | TAGGCTAGCCGCCACCATGGCCGAGAAGGCCGGATCTACC |
| | TTCAGCCACCTGCTGGTCCCTATTCTGCTGCTGATCGGCT |
| | GGATCGTGGGCTGCATCATCATGATCTACGTGGTGTTCAG |
| | CTGACGGCCGCGCGGATCCAGACATGATAAGATACATTGA |
| | TGAGTTTGGACAAACCACAACTAGAATGCAGTGAAAAAAA |
| | TGCTTTATTTGTGAAATTTGTGATGCTATTGCTTTATTTG |
| | TAACCATTATAAGCTGCAATAAACAAGTGAATTCCCGATT |
| | AGGATCTTCCTAGAGCATGGCTACGTAGATAAGTAGCATG |
| | GCGGGTTAATCATTAACTACAAGGAACCCCTAGTGATGGA |
| | GTTGGCCACTCCCTCTCTGCGCGCTCGCTCGCTCACTGAG |
| | GCCGGGCGACCAAAGGTCGCCCGACGCCCGGGCTTTGCCC |
| | GGGCGGCCTCAGTGAGCGAGCGAGCGCGCAG |
| | (SEQ ID NO: 74) |
| pHZ63 (human DWORF | CTGCGCGCTCGCTCGCTCACTGAGGCCGCCCGGGCAAAGC |
| version of pHZ34) | CCGGGCGTCGGGCGACCTTTGGTCGCCCGGCCTCAGTGAG |
| | CGAGCGAGCGCGCAGAGAGGGAGTGGCCAACTCCATCACT |
| | AGGGGTTCCTTGTAGTTAATGATTAACCCGCCATGCTACT |
| | TATCTACGTAGCCATGCTCTAGGAAGATCGGAATTCTCCC |
| | CAGCATGCCTGCTATTGTCTTCCCAATCCTCCCCCTTGCT |
| | GTCCTGCCCCACCCCACCCCCCAGAATAGAATGACACCTA |
| | CTCAGACAATGCGATGCAATTTCCTCATTTTATTAGGAAA |
| | GGACAGTGGGAGTGGCACCTTCCAGGGTCAAGGAAGGCAC |
| | GGGGGAGGGGCAAACAACAGATGGCTGGCAACTAGAAGGC |
| | ACAGTCGAGGCAGATCTCGAAGACGCGGAAGAGGCCGCAG |
| | AGCCGGCAGCAGGCCGCGGGAAGGAAGGTCCGCTGGATTG |
| | AGGGCCGAAGGGACGTAGCAGAAGGACGTCCCGCGCAGAA |
| | TCCAGGTGGCAACACAGGCGAGCAGCCAAGGAAAGGACGA |
| | TGATTTCCCCGACAACACCACGGAATTGTCAGTGCCCAAC |
| | AGCCGAGCCCCTGTCCAGCAGCGGGCAAGGCAGGCGGCGA |
| | TGAGTTCCGCCGTGGCAATAGGGAGGGGGAAAGCGAAAGT |
| | CCCGGAAAGGAGCTGACAGGTGGTGGCAATGCCCCAACCA |
| | GTGGGGGTTGCGTCAGCAAACACAGTGCACACCACGCCAC |
| | GTTGCCTGACAACGGGCCACAACTCCTCATAAAGAGACAG |
| | CAACCAGGATTTATACAAGGAGGAGAAAATGAAAGCCATA |
| | CGGGAAGCAATAGCATGATACAAAGGCATTAAAGCAGCGT |
| | ATCCACATAGCGTAAAAGGAGCAACATAGTTAAGAATACC |
| | AGTCAATCTTTCACAAATTTTGTAATCCAGAGGTTGATTG |
| | GATCCAAAGCTTCTAAGAGAAGACAACATAAATCATTATG |
| | ATGCAGCCCACAATCCAGCCAATCAGGAGAAGAATAGGAA |
| | CCAGAAGGTGTGAAAATGTAGACCCCGCTTTTTCAGCCAT |
| | GGTGGCGGGCGGCCGCTAGATCTGTATCGATTGGATCGGC |
| | CGCGGGTACAATTCCGCAGCTTTTAGAGCAGAAGTAACAC |
| | TTCCGTACAGGCCTGCAGAAAAGACCCAGGAAAGGAACAG |
| | TCTGTTAGTCTGTCAGCATGCATAAGAGCCAAAGGGGTGT |
| | GCCTATAGAGTCTATAGGCGGTACTTACCAGTTATGGAGG |
| | CGTCTGCTCAGTCTCAGCGGGGACTGGGTGAGGCAGAGGA |
| | TGGAGAGGGCTTTAAGCAGGCATGTGGGCTGGGGCCTGGT |
| | GAGCCAGCCCTGCGGAGGGAGGAATGTGCGACAGGGGACG |
| | GGTGGGGCAGGGGGATGGCGGTGGGGGTGGGGGGTGTTGG |
| | CTGCTATTTTGGCAGGTGCCAGGGACAAGGCTACAGGAAC |
| | ATGTACCCCACGCCATATAAGCCCATGTGGTCCTCCAGCT |
| | GCTCAGATAAGCTATTTAAAACCAGAGCAGATATGCAGGG |
| | AACAGTCATGCAACATAAACCAGCTGTCCCTCTTGAGAAT |
| | CCTGATAAAGCAGAGGCCAGCAACCCAGGCCTGGGAGGGC |
| | CAGCTGGGAGCAGGGTTGGGGGGCAGAAGGCAACCTCCAA |
| | GACACTCCATAAGTCTCAGCACCAGAATCTTGGAAGGCAG |
| | AGGGCAAGAGTTATGTGCTGCTCCACTTGAACTGATGCTG |
| | GGGGTAAAGACATCTTCCAGGCTACTGGCTCCTAATGGAC |
| | TGAGCAGCCTTAGGCAGGTTGCCGGCTCTGCCAGCCCCAG |
| | TGAGGACATCTGCAAGGTGGGTCTTCTCCATGACGACAGC |
| | AGCCCTGAGGTTTGCCCATGAAAGGTCTGCTGCCCTCGCC |
| | CCTCTGGCTCCAGGGCCTTTTTTTAGTCCTTGGGCACATT |
| | CCTCCTCCCCAAAGGGCCGATGGGCAGATAGAGGAGAGAC |
| | AGGACCGTCTCACACCACCTCCCCTACCCACATGGCCCTT |
| | ACCTTAGTTATTTTTAATCTGAAGGCTCGAGTTAAGGGCA |
| | GCCAGAAGTCAGATGCTCAAGGGGCTTCATGATGTCCCCA |
| | TAATTTTTGGCAGAGGGAAAAAGATCGGATCCTCAGGCGT |
| | AGTTCACCCCGTCCTCGAGGCCGCCCGGGTCGACTAAAAA |
| | ACCTCCCACACCTCCCCCTGAACCTGAAACATAAAATGAA |
| | TGCAATTGTTGTTGTTAAACTGGCCTGCCCGAGACCAAAC |
| | GTGCGGAACGTAGTTAAGTGTTAGAGGTAGGATTTGAAGC |
| | CTGTCGATCATTCTGATTCTCCTTTTCTCTACGTCTGCTT |
| | CCTGTCAATGGGCATCCTCACTGTCAAATGCAGATGGTAC |
| | AGCAGGGCTTGGTCTCAGCCAGGCAGGCCTCTCCCCAGTC |
| | TCCATGGCTCAGCTGTCCAGCAGTTTCATCCCTAGACCAT |
| | CCCAAACATGGTTGAGAAGCTCTGAGGGGAGGACCCAGCA |
| | CTGCCCGGCCCCTGAAGATAATCAGCAGTCCTGCTCAGCA |
| | TATCAATCCAAGCCCACTCTAGACAGAGATGCCGGTGCCC |
| | AGTTTTCTATTTTTAACTGGTGTGAACTGAAGGAAAAGCA |
| | CAGCATTAGAAGTCCAAGCAGGGATAAAAGCAGTCTGGGC |
| | TTTCACATGACAGCATCTGGGGCTGCGGCAGAGGGTCGGG |
| | TCCGAAGCGCTGCCTTATCAGCGTCCCCAGCCCTGGGAGG |
| | TGACAGCTGGCTGGCTTGTGTCAGCCCCTCGGGCACTCAC |
| | GTATCTCCGTCCGACGGGTTTAAAATAGCAAAACTCTGAG |
| | GCCACACAATAGCTTGGGCTTATATGGGCTCCTGTGGGGG |
| | AAGGGGGAGCACGGAGGGGGCCGGGGCCGCTGCTGCCAAA |
| | ATAGCAGCTCACAAGTGTTGCATTCCTCTCTGGGCGCCGG |
| | GCACATTCCTGCTGGCTCTGCCCGCCCCGGGGTGGGCGCC |
| | GGGGGGACCTTAAAGCCTCTGCCCCCCAAGGAGCCCTTCC |
| | CAGACAGCCGCCGGCACCCACCGCTCCGTGGGACGATCCC |
| | CGAAGCTCTAGAGCTTTATTGCGGTAGTTTATCACAGTTA |
| | AATTGCTAACGCAGTCAGTGCTTCTGACACAACAGTCTCG |
| | AACTTAAGCTGCAGAAGTTGGTCGTGAGGCACTGGGCAGG |
| | TAAGTATCAAGGTTACAAGACAGGTTTAAGGAGACCAATA |
| | GAAACTGGGCTTGTCGAGACAGAGAAGACTCTTGCGTTTC |
| | TGATAGGCACCTATTGGTCTTACTGACATCCACTTTGCCT |
| | TTCTCTCCACAGGTGTCCACTCCCAGTTCAATTACAGCTC |
| | TTAAGGCTAGAGTACTTAATACGACTCACTATAGGCTAGC |
| | CGCCACCATGGCCGAGAAGGCCGGATCTACCTTCAGCCAC |
| | CTGCTGGTCCCTATTCTGCTGCTGATCGGCTGGATCGTGG |
| | GCTGCATCATCATGATCTACGTGGTGTTCAGCTGACGGCC |
| | GCGCGGATCCAGACATGATAAGATACATTGATGAGTTTGG |
| | ACAAACCACAACTAGAATGCAGTGAAAAAAATGCTTTATT |
| | TGTGAAATTTGTGATGCTATTGCTTTATTTGTAACCATTA |
| | TAAGCTGCAATAAACAAGTGAATTCCCGATTAGGATCTTC |
| | CTAGAGCATGGCTACGTAGATAAGTAGCATGGCGGGTTAA |
| | TCATTAACTACAAGGAACCCCTAGTGATGGAGTTGGCCAC |
| | TCCCTCTCTGCGCGCTCGCTCGCTCACTGAGGCCGGGCGA |
| | CCAAAGGTCGCCCGACGCCCGGGCTTTGCCCGGGCGGCCT |
| | CAGTGAGCGAGCGAGCGCGCAG (SEQ ID NO: 75) |

### Vectors

In some aspects, the disclosure provides vectors comprising the expression cassettes provided herein. The vector can be any viral vector or any non-viral vector known in the art or described herein.

In some embodiments, the vector is a viral vector. In some embodiments the viral vector is an adeno-associated virus vector (AAV), an adenoviral vector (AV), a lentiviral vector (LV), a retroviral vector (RV), a herpes simplex virus vector (HSV), or a poxvirus vector.

In some embodiments, provided herein is an AAV comprising any expression cassette described herein. In some embodiments, provided herein is an AV comprising any expression cassette described herein. In some embodiments, provided herein is an LV comprising any expression cassette described herein. In some embodiments, provided herein is an RV comprising any expression cassette described herein. In some embodiments, provided herein is an HSV comprising any expression cassette described herein. In some embodiments, provided herein is a poxvirus-based vector comprising any expression cassette described herein.

In some embodiments, the vector is a non-viral vector. In some embodiments, the non-viral vector is a naked DNA (e.g., a DNA plasmid). In some embodiments, the non-viral vector is a plasmid. In some embodiments, the non-viral vector is a liposome or lipid vector comprising plasmid DNA and a lipid solution.

For example, viral and non-viral vectors and delivery systems are described in Sung & Kim 2019, Biomaterials Research 23:8; Mali, 2013, Indian Journal of Human Genetics, 19(1):3-8; Hardee et al., 2017, Genes 8:65; Bulcha et al., 2020, Signal Transduction and Targeted Therapy; Ghosh et al., 2020, Applied Biosafety: Journal of ABSA International 25(1):7-18, the disclosures of each of which are hereby incorporated by reference herein in their entireties.

In some embodiments, the vectors are recombinant vectors.

In some embodiments, the vectors described herein comprise an expression cassette comprising a polynucleotide encoding any gene product described herein. In some embodiments, the expression cassette comprises a sequence that shares at least 75%, 80%, 90%, 95%, 96%, 97%, 98%, 99%, or 100% identity to SEQ ID NOS: 201, 203, 205, 207, 209, 211, 213, 215, 217, 219, 221, 223, 225,

In some embodiments, the vectors described herein comprise an expression cassette comprises a polynucleotide encoding DWORF. In some embodiments, the expression cassette comprises a polynucleotide sequence that shares at least 75%, 80%, 90%, 95%, 96%, 97%, 98%, 99%, or 100% identity to SEQ ID NOs: 20-24 and SEQ ID NOs: 45-75.

In some aspects of the disclosure, a vector is used to deliver the expression cassettes described herein to cardiac cells of a subject, e.g., to treat cardiomyopathy. In some embodiments, the disclosure provides a viral vector comprising an expression cassette comprising a polynucleotide encoding a gene product (such as any gene product described herein, e.g., a DWORF polypeptide) operatively linked to a promoter and a pharmaceutically acceptable carrier. In some embodiments, the disclosure provides a virion comprising a capsid and an expression cassette comprising a polynucleotide encoding a gene product (such as any gene product described herein, e.g., a DWORF polypeptide) operatively linked to a promoter and a pharmaceutically acceptable carrier. In some embodiments, the disclosure provides a plasmid comprising an expression cassette comprising a polynucleotide encoding a gene product (such as any gene product described herein, e.g., a DWORF polypeptide) operatively linked to a promoter and a pharmaceutically acceptable carrier.

In some embodiments, the viral vectors described herein are replication incompetent, in that it cannot independently further replicate and package its genome. For example, when a cardiac cell is targeted with a virion, the transgene is expressed in the targeted cardiac cell, however, due to the fact that the targeted cardiac cell lacks packaging and accessory function genes, the virion is not able to replicate. In some embodiments, the viral vectors described herein are replication-competent.

In some embodiments, the vectors described herein are capable of being delivered to both dividing and non-dividing cells. In some embodiments, the vectors described herein are capable of being delivered to non-dividing cells. In some embodiments, the vectors described herein are capable of being delivered to dividing cells.

In some embodiments, the vectors comprising the expression cassettes described herein lead to cardiac cell-specific expression of a transgene. In some embodiments, the vectors comprising the expression cassettes described herein lead to cardiomyocyte-specific expression of a transgene. In some embodiments, the vectors comprising the expression cassettes described herein allow high expression of a transgene in a cardiac cell (e.g., a cardiomyocyte) and low or no expression in other cells (e.g., low or no expression in liver cells, low or no expression in muscle cells except for muscle cells of the heart, low or no expression in cardiac fibroblasts). In some embodiments, the vectors comprising the expression cassettes described herein allow high expression of a transgene in heart tissue of a subject (e.g., in human heart). In some embodiments, the vectors comprising the expression cassettes described herein allow no or low expression of a transgene in tissues of a subject other than the heart (e.g., in liver or in muscles except those of the heart). “High” and “low” can be relative to each other, for example, the expression of a transgene in cardiac cells (e.g., cardiomyocytes) and/or heart tissue can be at least 2 fold, 5 fold, 10 fold, 15 fold, 20 fold, 50 fold, 100 fold, 150 fold, or 200 fold higher than its expression in other cells and tissues (e.g., liver, muscle except for the heart).

### Recombinant AAV Virions

In some aspects, the disclosure provides recombinant AAV (rAAV) virions comprising the expression cassettes provided herein. In some embodiments, the rAAV virion comprises a capsid protein and an expression cassette. In some embodiments, the expression cassette comprises a polynucleotide encoding any gene product described herein.

In some embodiments, the expression cassette comprises a polynucleotide encoding DWORF. In some embodiments, the expression cassette comprises a polynucleotide sequence that shares at least 75%, 80%, 90%, 95%, 96%, 97%, 98%, 99%, or 100% identity to SEQ ID NOs: 20-24 and SEQ ID NOs: 45-63. In some embodiments, the expression cassette comprises a polynucleotide sequence that shares at least 75%, 80%, 90%, 95%, 96%, 97%, 98%, 99%, or 100% identity to SEQ ID NO: 61. In some embodiments, the expression cassette comprises SEQ ID NO: 61. In some embodiments, the expression cassette comprises a polynucleotide sequence that shares at least 75%, 80%, 90%, 95%, 96%, 97%, 98%, 99%, or 100% identity to SEQ ID NO: 62. In some embodiments, the expression cassette comprises SEQ ID NO: 62. In some embodiments, the expression cassette comprises a polynucleotide sequence that shares at least 75%, 80%, 90%, 95%, 96%, 97%, 98%, 99%, or 100% identity to SEQ ID NO: 63. In some embodiments, the expression cassette comprises SEQ ID NO: 63.

In some aspects of the disclosure, an rAAV virion is used to deliver the expression cassettes described herein to cardiac cells of a subject, e.g., to treat cardiomyopathy. Accordingly, the disclosure provides an rAAV virion, the rAAV virion comprising an AAV capsid and an expression cassette comprising a polynucleotide encoding a DWORF polypeptide operatively linked to a promoter and a pharmaceutically acceptable carrier.

The rAAV virions of the disclosure comprise a capsid protein. Capsid proteins are structural proteins that make up the assembled icosahedral packaging of the rAAV virion that contains the expression cassette. Capsid proteins are classified by the serotype. Wild type capsid serotypes in rAAV virions can be, for example, AAV1, AAV2, AAV3, AAV4, AAV5, AAV6, AAV7, AAV8, AAV9, AAV10, AAV11, or AAV12 (Naso et al. BioDrugs 31:317-334 (2017)). Engineered capsid types include chimeric capsids and mosaic capsids (Choi et al. Curr Gene Ther. 5: 299-310 (2005)). Capsids are selected for rAAV virions based on their ability to transduce specific tissue or cell types (Liu et al. Curr Pharm Des. 21:3248-56 (2015)).

Any capsid protein that can facilitate rAAV virion transduction into cardiac cells for delivery of a transgene, as described herein, can be used. Capsid proteins used in rAAV virions for transgene delivery to cardiac cells that result in high expression can be, for example, AAV4, AAV6, AAV7, AAV8, and AAV9 (Zincarelli et al. Mol. Ther. 16:P1073-1080 (2008)). Artificial capsids, such as chimeric capsids generated through combinatorial libraries, can also be used for transgene delivery to cardiac cells that results in high expression (see U.S. 63/012,703, the contents of which are herein incorporated by reference). Other capsid proteins with various features can also be used in the rAAV virions of the disclosure. AAV vectors and capsids are provided in U.S. Pat. Pub. Nos. U.S. Ser. No. 10/011,640B2; U.S. Pat. No. 7,892,809B2, U.S. Pat. No. 8,632,764B2, U.S. Pat. No. 8,889,641B2, U.S. Pat. No. 9,475,845B2, U.S. Ser. No. 10/889,833B2, U.S. Ser. No. 10/480,011B2, and U.S. Ser. No. 10/894,949B2, the contents of which are herein incorporated by reference; and Int'l Pat. Pub. Nos. WO2020198737A1, WO2019028306A2, WO2016054554A1, WO2018152333A1, WO2017106236A1, WO2008124724A1, WO2017212019A1, WO2020117898A1, WO2017192750A1, WO2020191300A1, and WO2017100671A1, the contents of which are herein incorporated by reference.

In some embodiments, the rAAV virions of the disclosure comprise an engineered capsid protein. Engineered capsid proteins can be derived from a parental, e.g., wild type, capsid and include, for example, variant polypeptide sequence with respect to a parental capsid sequence at one or more sites. For example, variant sites of the parental capsid can occur at the VR-IV site, VR-V site, VR-VII site and/or VR-VIII site (see, e.g., Bining and Srivastava. Mol Ther Methods Clin Dev. 12:248-265 (2019)).

In some embodiments, the capsid protein is an AAV5/AAV9 chimeric capsid protein. In some embodiments, the chimeric capsid protein comprises at least 1, 2, 3, 4, 5 or more polypeptide segments that are derived from AAV5 capsid protein (SEQ ID NO. 144). In some embodiments, the chimeric capsid protein comprises at least 1, 2, 3, 4, 5 or more polypeptide segments that are derived from AAV9 capsid protein (SEQ ID NO: 143). In some embodiments, at least one polypeptide segment is derived from the AAV5 capsid protein and at least one polypeptide segment is derived from the AAV9 capsid protein.

In some embodiments, the capsid protein is a combinatory capsid proteins. As used herein, “combinatory capsid protein” refers to a AAV5/AAV9 chimeric capsid protein, which further comprises amino acid variations with respect to the chimeric parental sequence at one or more sites. In some embodiments, the one or more sites of the chimeric parental sequence are selected from those equivalent to the VR-IV site, the VR-V site, the VR-VII site and the VR-VIII site of the AAV9 capsid protein.

In some embodiments, the rAAV virions comprise an engineered capsid protein selected from Table 7.

TABLE 7
Engineered Capsid Proteins

| Engineered | |
|---|---|
| Capsid | SEQ ID NO: |
| CR9-01 | 145 |
| CR9-07 | 146 |
| CR9-08 | 147 |
| CR9-09 | 148 |
| CR9-10 | 149 |
| CR9-11 | 150 |
| CR9-13 | 151 |
| CR9-14 | 152 |
| CR9-15 | 153 |
| CR9-16 | 154 |
| CR9-17 | 155 |
| CR9-20 | 156 |
| CR9-21 | 157 |
| CR9-22 | 158 |
| ZC23 | 159 |
| ZC24 | 160 |
| ZC25 | 161 |
| ZC26 | 162 |
| ZC27 | 163 |
| ZC28 | 164 |
| ZC29 | 165 |
| ZC30 | 166 |
| ZC31 | 167 |
| ZC32 | 168 |
| ZC33 | 169 |
| ZC34 | 170 |
| ZC35 | 171 |
| ZC40 | 172 |
| ZC41 | 173 |
| ZC42 | 174 |
| ZC43 | 175 |
| ZC44 | 176 |
| ZC45 | 177 |
| ZC46 | 178 |
| ZC47 | 179 |
| ZC48 | 180 |
| ZC49 | 181 |
| ZC50 | 182 |
| TN47-07 | 183 |
| TN47-10 | 184 |
| TN47-13 | 185 |
| TN47-14 | 186 |
| TN47-17 | 187 |
| TN47-22 | 188 |
| TN40-07 | 189 |
| TN40-10 | 190 |
| TN40-13 | 191 |
| TN40-14 | 192 |
| TN40-17 | 193 |
| TN40-22 | 194 |
| TN44-07 | 195 |
| TN44-10 | 196 |
| TN44-13 | 197 |
| TN44-14 | 198 |
| TN44-17 | 199 |
| TN44-22 | 200 |

In some embodiments, the rAAV is replication defective, in that the rAAV virion cannot independently further replicate and package its genome. For example, when a cardiac cell is targeted with rAAV virions, the transgene is expressed in the targeted cardiac cell, however, due to the fact that the targeted cardiac cell lacks AAV rep and cap genes and accessory function genes, the rAAV is not able to replicate.

In some embodiments, rAAV virions of the present disclosure encapsulating the expression cassettes as described herein, can be produced using helper-free production. rAAVs are replication-deficient viruses and normally require components from a live helper virus, such as adenovirus, in a host cell for packaging of infectious rAAV virions. rAAV helper-free production systems allow the production of infectious rAAV virions without the use of a live helper virus. In the helper-free system, a host packaging cell line is co-transfected with three plasmids. A first plasmid may contain adenovirus gene products (e.g., E2A, E4, and VA RNA genes) needed for the packaging of rAAV virions. A second plasmid may contain required AAV genes (e.g., REP and CAP genes). A third plasmid contains the polynucleotide sequence encoding the transgene of interest and a promoter flanked by ITRs. A host packaging cell line can be, for example, AAV-293 host cells. Suitable host cells contain additional components required for packaging infectious rAAV virions that are not supplied by the plasmids. In some embodiments, the CAP genes can encode, for example, AAV capsid proteins as described herein.

In some embodiments, the CAP genes can encode, for example, AAV capsid proteins as described herein. In some embodiments, the promoter is a promoter sequence as described herein. In some embodiments, the promoter sequence is a cTnT promoter sequence. In some embodiments, the polypeptide of interest is a DWORF polypeptide.

The expression cassettes, enhancers and/or promoters described herein with respect to AAV virions need not be limited to their use in AAV virions and can be incorporated in essentially any other construct where expression of a polynucleotide encoding a gene product is desired.

rAAV virions can deliver transgenes to cells in a subject that are, in turn, expressed in the cell. A transgene delivered by an rAAV virion may be incorporated into the genome of the targeted cell, allowing for potential long-term expression of the transgene product. Compared to other viral transgene delivery systems, such as adenoviruses, rAAV virions have the advantage of low immunogenicity. rAAV virions can be used to transduce and deliver transgenes to many cells types, including eye, blood, liver, heart, joint tissue, muscle, brain kidney or lung cells (U.S. Pat. No. 10,308,957; U.S. Pat. No. 9,803,218). rAAV virions can contain genomes up to about 5.2 kilobases (kb), limiting the size of the polynucleotide that can be integrated into the host cell to about 4.4 kb (Choi et al. Mol Brain. 7:1 (2014)).

### Methods of Use

### Methods of Increasing Polypeptide Expression

The disclosure provides methods of increasing polypeptide expression in a cell comprising contacting the cell with any vector or virion (e.g., rAAV virion) described herein. In some embodiments, the cell is a cardiac cell. In some embodiments, the cell is a cardiomyocyte. In some embodiments, the contacting is in vitro. In some embodiments, the contacting is in vivo. In some embodiments, the polypeptide is any polypeptide for use in treating or preventing a heart disease. In some embodiments, the polypeptide is any polypeptide described herein. In some embodiments, the polypeptide is encoded by any transgene described herein.

The disclosure provides methods of increasing polypeptide expression in a tissue comprising contacting the tissue with any vector or virion (e.g., rAAV virion) described herein. In some embodiments, the tissue is cardiac tissue. In some embodiments, the contacting is in vitro. In some embodiments, the contacting is in vivo.

The disclosure provides methods of increasing polypeptide expression in an organ comprising contacting the organ with any vector or virion (e.g., rAAV virion) described herein. In some embodiments, the organ is a heart. In some embodiments, the heart is diseased or at risk of disease. In some embodiments, the heart has borderline or reduced ejection fraction. In some embodiments, the heart has a normal ejection fraction. In some embodiments, the heart comprises a genetic mutation associated with a heart disease. In some embodiments, the genetic mutation is a PLN mutation. In some embodiments, the heart has low or undetectable polypeptide expression compared to a healthy heart. In some embodiments, the contacting is in vitro. In some embodiments, the contacting is in vivo.

The disclosure provides methods of increasing polypeptide expression in a subject comprising administering to the subject any vector or virion (e.g., rAAV virion) described herein. In some embodiments the subject is an animal. An animal can be, without limitation, a mouse, rat, dog, or non-human primate. In some embodiments, the subject is a human. In some embodiments, the increased polypeptide expression is in the heart of the subject. In some embodiments, the subject has a heart disease or is at risk of a heart disease. In some embodiments, the subject has borderline or reduced ejection fraction. In some embodiments, the subject has a normal ejection fraction. In some embodiments, the subject has a genetic mutation associated with a heart disease. In some embodiments, the genetic mutation is a PLN mutation. In some embodiments, the subject has low or undetectable level of DWORF expression compared to a healthy subject.

In some embodiments, the polypeptide is expressed in a cell, tissue, organ, or subject at a desired level of expression. A “desired level of expression” can be selected such that the level of expression is relative to the polypeptide expression in a healthy or diseased cell, tissue, organ, or subject. For example, an increased level in polypeptide expression relative to a diseased cardiac cell, cardiac tissue, heart, or subject with a heart disease or disorder can be a desired level of expression. The desired level of expression can be expressed relative to the difference in expression achieved between different vector or virions (e.g., rAAV virions) containing different expression cassettes. For example, a vector or virion (e.g., rAAV virion) comprising an expression cassette comprising a promoter and an enhancer may achieve a desired level of expression compared to a vector or virion (e.g., rAAV virion) comprising only a promoter.

The polypeptide expression level achieved by any vector or virion (e.g., rAAV virion) comprising an expression cassette can be described as a “fold” change (i.e., increase or decrease) compared to basal polypeptide expression. The polypeptide expression level achieved by a vector or virion (e.g., rAAV virion) comprising an expression cassette can be described as a “fold change” compared to polypeptide expression achieved by an expression cassette comprising a single promoter, no enhancers, and a sequence encoding the polypeptide. Fold change is a relative quantity, such that the levels of expression between the expression level achieved by an expression cassette and reference expression level are expressed as a ratio. It is understood that when describing fold change of polypeptide expression, “about” refers to ±0.5-fold. Polypeptide expression levels can be categorized as “low expression”, “medium expression”, or “high expression.” “Low expression” is meant to include expression levels between about 1.5-fold and 20-fold increase in polypeptide expression. “Medium expression” is meant to include expression levels between about 20-fold increase and about 60-fold increase in polypeptide expression. “High expression” is meant to include expression levels between about 60-fold and 140-fold increase in polypeptide expression.

In some embodiments, the polypeptide expression level is between about a 1.5-fold and 150-fold increase. In some embodiments, the polypeptide expression level is increased at least about 1.5-fold, about 3.5-fold, about 5.5-fold, about 7.5-fold, about 9.5-fold, about 11.5-fold, about 13.5-fold, about 15.5-fold, about 17.5-fold, about 19.5-fold, about 21.5-fold, about 23.5-fold, about 25.5-fold, about 27.5-fold, about 29.5-fold, about 31.5-fold, about 33.5-fold, about 35.5-fold, about 37.5-fold, about 39.5-fold, about 41.5-fold, about 43.5-fold, about 45.5-fold, about 47.5-fold, about 49.5-fold, about 51.5-fold, about 53.5-fold, about 55.5-fold, about 57.5-fold, about 59.5-fold, about 61.5-fold, about 63.5-fold, about 65.5-fold, about 67.5-fold, about 69.5-fold, about 71.5-fold, about 73.5-fold, about 75.5-fold, about 77.5-fold, about 79.5-fold, about 81.5-fold, about 83.5-fold, about 85.5-fold, about 87.5-fold, about 89.5-fold, about 91.5-fold, about 93.5-fold, about 95.5-fold, about 97.5-fold, about 99.5-fold, about 101.5-fold, about 103.5-fold, about 105.5-fold, about 107.5-fold, about 109.5-fold, about 111.5-fold, about 113.5-fold, about 115.5-fold, about 117.5-fold, about 119.5-fold, about 121.5-fold, about 123.5-fold, about 125.5-fold, about 127.5-fold, about 129.5-fold, about 131.5-fold, about 133.5-fold, about 135.5-fold, about 137.5-fold, about 139.5-fold, about 141.5-fold, about 143.5-fold, about 145.5-fold, about 147.5-fold, or about 149.5-fold.

In some embodiments, the polypeptide expression level is increased at least or more than about 5-fold. In some embodiments, the polypeptide expression level is increased at least or more than about 10-fold. In some embodiments, the polypeptide expression level is increased at least or more than about 15-fold. In some embodiments, the polypeptide expression level is increased at least or more than about 25-fold. In some embodiments, the polypeptide expression level is increased at least or more than about 35-fold. In some embodiments, the polypeptide expression level is increased at least or more than about 50-fold. In some embodiments, the polypeptide expression level is increased at least or more than about 60-fold. In some embodiments, the polypeptide expression level is increased at least or more than about 75-fold. In some embodiments, the polypeptide expression level is increased at least or more than about 85-fold. In some embodiments, the polypeptide expression level is increased at least or more than about 100-fold. In some embodiments, the polypeptide expression level is increased at least or more than about 110-fold. In some embodiments, the polypeptide expression level is increased at least or more than about 125-fold.

In some embodiments, the fold increase is relative to an expression cassette comprising a single promoter, no enhancers, and a sequence encoding the polypeptide. In some embodiments, the fold increase is relative to a healthy cell, tissue, organ, or subject. In some embodiments, the fold increase is relative to a diseased cell, tissue, organ, or subject.

### Methods of Increasing DWORF Expression

The disclosure provides methods of increasing DWORF expression in a cell comprising contacting the cell with the rAAV virions described herein. In some embodiments, the cell is a cardiac cell. In some embodiments, the cell is a cardiomyocyte. In some embodiments, the contacting is in vitro. In some embodiments, the contacting is in vivo.

The disclosure provides methods of increasing DWORF expression in a tissue comprising contacting the tissue with the rAAV virions described herein. In some embodiments, the tissue is cardiac tissue. In some embodiments, the contacting is in vitro. In some embodiments, the contacting is in vivo.

The disclosure provides methods of increasing DWORF expression in an organ comprising contacting the organ with the rAAV virions described herein. In some embodiments, the organ is a heart. In some embodiments, the heart is diseased or at risk of disease. In some embodiments, the heart has borderline or reduced ejection fraction. In some embodiments, the heart has a normal ejection fraction. In some embodiments, the heart comprises a genetic mutation associated with a heart disease. In some embodiments, the genetic mutation is a PLN mutation. In some embodiments, the heart has low or undetectable DWORF expression compared to a healthy heart. In some embodiments, the contacting is in vitro. In some embodiments, the contacting is in vivo.

The disclosure provides methods of increasing DWORF expression in a subject comprising administering to the subject the rAAV virions described herein. In some embodiments the subject is an animal. An animal can be, without limitation, a mouse, rat, dog, or non-human primate. In some embodiments, the subject is a human. In some embodiments, the increased DWORF expression is in the heart of the subject. In some embodiments, the subject has a heart disease or is at risk of a heart disease. In some embodiments, the subject has borderline or reduced ejection fraction. In some embodiments, the subject has a normal ejection fraction. In some embodiments, the subject has a genetic mutation associated with a heart disease. In some embodiments, the genetic mutation is a PLN mutation. In some embodiments, the subject has low or undetectable level of DWORF expression compared to a healthy subject.

In some embodiments, DWORF is expressed in a cell, tissue, organ, or subject at a desired level of expression. A “desired level of expression” can be selected such that the level of expression is relative to DWORF expression in a healthy or diseased cell, tissue, organ, or subject. For example, an increased level in DWORF expression relative to a diseased cardiac cell, cardiac tissue, heart, or subject with a heart disease or disorder can be a desired level of expression. The desired level of expression can be expressed relative to the difference in expression achieved between different rAAV virions containing different expression cassettes. For example, an rAAV virion comprising an expression cassette comprising a promoter and an enhancer may achieve a desired level of expression compared to an rAAV virion comprising only a promoter.

The DWORF expression level achieved by an rAAV virion comprising an expression cassette can be described as a “fold” change (i.e., increase or decrease) compared to basal DWORF expression. The DWORF expression level achieved by an rAAV virion comprising an expression cassette can be described as a “fold change” compared to DWORF expression achieved by an expression cassette comprising a single promoter, no enhancers, and a sequence encoding DWORF. Fold change is a relative quantity, such that the levels of expression between the expression level achieved by an expression cassette and reference expression level are expressed as a ratio. It is understood that when describing fold change of DWORF expression, “about” refers to ±0.5-fold. DWORF expression levels can be categorized as “low expression”, “medium expression”, or “high expression.” “Low expression” is meant to include expression levels between about 1.5-fold and 20-fold increase in DWORF expression. “Medium expression” is meant to include expression levels between about 20-fold increase and about 60-fold increase in DWORF expression. “High expression” is meant to include expression levels between about 60-fold and 140-fold increase in DWORF expression.

In some embodiments, the DWORF expression level is between about a 1.5-fold and 150-fold increase. In some embodiments, the DWORF expression level is increased about 1.5-fold, about 3.5-fold, about 5.5-fold, about 7.5-fold, about 9.5-fold, about 11.5-fold, about 13.5-fold, about 15.5-fold, about 17.5-fold, about 19.5-fold, about 21.5-fold, about 23.5-fold, about 25.5-fold, about 27.5-fold, about 29.5-fold, about 31.5-fold, about 33.5-fold, about 35.5-fold, about 37.5-fold, about 39.5-fold, about 41.5-fold, about 43.5-fold, about 45.5-fold, about 47.5-fold, about 49.5-fold, about 51.5-fold, about 53.5-fold, about 55.5-fold, about 57.5-fold, about 59.5-fold, about 61.5-fold, about 63.5-fold, about 65.5-fold, about 67.5-fold, about 69.5-fold, about 71.5-fold, about 73.5-fold, about 75.5-fold, about 77.5-fold, about 79.5-fold, about 81.5-fold, about 83.5-fold, about 85.5-fold, about 87.5-fold, about 89.5-fold, about 91.5-fold, about 93.5-fold, about 95.5-fold, about 97.5-fold, about 99.5-fold, about 101.5-fold, about 103.5-fold, about 105.5-fold, about 107.5-fold, about 109.5-fold, about 111.5-fold, about 113.5-fold, about 115.5-fold, about 117.5-fold, about 119.5-fold, about 121.5-fold, about 123.5-fold, about 125.5-fold, about 127.5-fold, about 129.5-fold, about 131.5-fold, about 133.5-fold, about 135.5-fold, about 137.5-fold, about 139.5-fold, about 141.5-fold, about 143.5-fold, about 145.5-fold, about 147.5-fold, or about 149.5-fold.

In some embodiments, the DWORF expression level is increased at least or more than about 5-fold. In some embodiments, the DWORF expression level is increased at least or more than about 10-fold. In some embodiments, the DWORF expression level is increased at least or more than about 15-fold. In some embodiments, the DWORF expression level is increased at least or more than about 25-fold. In some embodiments, the DWORF expression level is increased at least or more than about 35-fold. In some embodiments, the DWORF expression level is increased at least or more than about 50-fold. In some embodiments, the DWORF expression level is increased at least or more than about 60-fold. In some embodiments, the DWORF expression level is increased at least or more than about 75-fold. In some embodiments, the DWORF expression level is increased at least or more than about 85-fold. In some embodiments, the DWORF expression level is increased at least or more than about 100-fold. In some embodiments, the DWORF expression level is increased at least or more than about 110-fold. In some embodiments, the DWORF expression level is increased at least or more than about 125-fold.

In some embodiments, the fold increase is relative to an expression cassette comprising a single promoter, no enhancers, and a sequence encoding DWORF. In some embodiments, the fold increase is relative to a healthy cell, tissue, organ, or subject. In some embodiments, the fold increase is relative to a diseased cell, tissue, organ, or subject.

### Methods of Treatment

In an aspect, any vector comprising an expression cassette described herein may be used for treating disease, such as heart disease.

In an aspect, rAAV virions comprising an expression cassette described herein may be used for treating disease (Wang et al. Nat Rev Drug Discov. 18:358-378 (2019)). For treatment, rAAV virions have been used to deliver transgenes encoding polypeptides such as microdystrophin (Chamberlain et al. Mol Ther. 25:1125-1131 (2017)), glial cell line-derived neurotrophic factor (McFarthing et al. J Parkinsons Dis. 9:251-264 (2019)), and Factor IX (Nathwani et al. N Engl J Med. 371:1994-2004 (2014)).

A variety of strategies for treating heart failure using rAAV-based delivery of a transgene have been pursued in vivo. In a pig model of heart failure, β-adrenergic receptor, a regulator of contractility, has been targeted by delivery of a small polypeptide, βARKct that indirectly prevents disruption of β-adrenergic receptor signaling (Raake et al. Eur Heart J. 34:1437-47 (2013)). In a canine model, cardiomyocyte viability was enhanced by rAAV-based delivery of a vascular endothelial growth factor (VEGF) isoform. In human clinical trials, rAAV-based delivery of an isoform of the SERCA calcium pump, SERCA2a, to the heart was tested as a treatment for heart failure. SERCA, or sarco/endoplasmic reticulum Ca²⁺-ATPase, or SR Ca²⁺-ATPase, is a calcium ATPase-type P-ATPase. SERCA resides in the sarcoplasmic reticulum (SR) within muscle cells. It is a Ca²⁺ ATPase that transfers Ca²⁺ from the cytosol of the cell to the lumen of the SR at the expense of ATP hydrolysis during muscle relaxation. SERCA activity is necessary for proper contractile function of the heart. However, direct replacement of SERCA activity by rAAV-based delivery of the SERCA2a isoform failed to show a significant effect in clinical trials (Bass-Stringer et al. Heart, Lung and Circulation. 27:1285-1300 (2018)). Enhancing SERCA activity using alternative strategies is desired for treating diseases of the heart, e.g., heart failure and cardiomyopathy.

There are three major domains on the cytoplasmic face of SERCA: the phosphorylation and nucleotide-binding domains, which form the catalytic site, and the actuator domain, which is involved in the transmission of major conformational changes. The rate at which SERCA moves Ca²⁺ across the SR membrane can be controlled by the regulatory protein phospholamban (PLN). SERCA is normally inhibited by PLN, with which it is closely associated. Increased β-adrenergic stimulation reduces the association between SERCA and PLN by the phosphorylation of PLN by PKA. When PLN is associated with SERCA, the rate of Ca²⁺ movement is reduced; upon dissociation of PLN, Ca²⁺ movement increases.

An alternative strategy to enhancing SERCA activity by delivering a SERCA2a isoform is to enhance activity of natively expressed SERCA by displacing PLN. Contacting SERCA with the DWORF polypeptide, described in detail above, can displace PLN and enhance SERCA activity.

In some embodiments, the disclosure provides a method of treating a heart disease or disorder in a subject in need thereof, the method comprising administering an effective amount of a vector comprising an expression cassette comprising a polynucleotide encoding a therapeutic polypeptide operatively linked to a promoter, wherein the therapeutic polypeptide can be any polypeptide useful for treating heart disease. As described herein, the vector can be any viral or non-viral vector.

In some embodiments, the disclosure provides a method of treating a heart disease or disorder in a subject in need thereof, the method comprising administering an effective amount of a recombinant adeno-associated virus (rAAV) virion, the rAAV virion comprising an AAV capsid and an expression cassette comprising a polynucleotide encoding a DWORF polypeptide operatively linked to a promoter.

In a method of treating a subject as described herein, “treating” or “treatment of a condition or subject in need thereof” refers to (1) taking steps to obtain beneficial or desired results, including clinical results such as the reduction of symptoms; (2) inhibiting the disease, for example, arresting or reducing the development of the disease or its clinical symptoms; (3) relieving the disease, for example, causing regression of the disease or its clinical symptoms; or (4) delaying the disease. For purposes of the methods described herein, beneficial or desired clinical results include, but are not limited to, reduction of symptoms associated with heart failure, cardiomyopathy, dilated cardiomyopathy, myocardial infarction, acute myocardial infarction, and chronic myocardial infarction.

In other aspects, the disclosure provides a method of preventing a heart disease or disorder in a subject in need thereof, the method comprising administering an effective amount of a vector comprising an expression cassette comprising a polynucleotide encoding a therapeutic polypeptide operatively linked to a promoter, wherein the therapeutic polypeptide can be any polypeptide useful for preventing heart disease. As described herein, the vector can be any viral or non-viral vector. In some embodiments, prevention of a disease causes the clinical symptoms of the disease not to develop in a patient that may be predisposed to the disease, but does not yet experience or display symptoms of the disease.

Subjects in need of treatment using the compositions and methods of the present disclosure include, but are not limited to, a subject suffering from or being at risk of heart failure. A subject “suffering from” heart failure is considered to have symptoms associated with or a confirmed diagnosis of any of the heart diseases described herein. A subject “at risk of” heart failure is considered to have one or more risk factors associated with any of the heart diseases described herein.

In some embodiments, the methods described herein are useful to treat heart disease or disorder with reduced ejection fraction (HFrEF). In some embodiments, the methods described herein are useful to treat heart disease or disorder with preserved ejection fraction (HFpEF).

In some embodiments, the methods described herein are useful to treat cardiomyopathy. In some embodiments, a method described herein is useful to treat dilated cardiomyopathy. In some embodiments, the subject suffers from or is at risk for cardiomyopathy. In some embodiments, the cardiomyopathy is dilated cardiomyopathy (DCM). In some embodiments, the DCM is genetic DCM (e.g., DCM associated with a PLN mutation in a subject to be treated). In some embodiments, the methods described herein are useful to treat PLN mutation-associated cardiomyopathy. In some embodiments, the DCM is non-genetic DCM. In some embodiments, subject suffers from or is at risk for myocardial infarction. In some embodiments, the myocardial infarction is chronic myocardial infarction. In some embodiments, the myocardial infarction is acute myocardial infarction.

Cardiomyopathy phenotypes can manifest in a subject through a multitude of molecular mechanisms. Transgenic animals have been developed to investigate the molecular pathophysiology of specific mechanisms and the efficacy of potential therapeutic and prevention strategies for cardiomyopathy phenotypes (Law et al. J Clin Med. 9:520 (2020)). These animal models can be used to evaluate aspects of the rAAV viral genomes, rAAV virions, and compositions thereof described herein. The MLP^{-/-} transgenic mouse model, for example, recapitulates the phenotype of dilated cardiomyopathy by knocking out the muscle LIM protein, a positive regulator of myogenic differentiation associated with the actin-based cytoskeleton. The absence of LIM protein results in a disruption of cytoskeletal architecture and decreased Ca²⁺ cycling (Minamisawa et al. Cell. 99:313-22 (1999)). Artificial replacement with a phosphomimetic PLN transgene delivered by rAAV reduced DCM symptoms in the animals, including improved ejection fraction (Iwanaga Y et al. J Clin Invest. 113:727-736 (2004). While the MLP^{-/-} model recapitulates DCM phenotype in a general way, other transgenic mouse models of DCM are more appropriate for specific DCM phenotypes, such as those driven by a mutation to the PLN gene. For example, a transgenic mouse model to recapitulate the clinically observed PLN-R14Del mutation has been developed (Haghighi et al. Proc. Natl. Acad. Sci. U.S.A 103:1388-1393 (2006)). It is expected that mouse models with different transgenic modifications to induce a DCM phenotype are not interchangeable for the purpose of evaluating the efficacy of a given therapeutic or prevention strategy, and that each model provides different information about the translation of a therapy.

In some embodiments, the subject in need of treatment has an inherited risk allele (i.e., mutation) for a heart disease or disorder. A risk allele can be, for example, a mutation to the PLN gene. Mutations to the PLN gene can cause a dysfunctional inhibitory effect on SERCA activity. Clinically observable mutations in the PLN gene and protein include a mutation in the PLN promoter, a truncation resulting in a PLN^{L39stop} mutant, aberrant R9C, R9L, and R9H mutations, PLN gene duplications, and deletion of arginine 14 (R14del) in the regulatory domain of PLN. Each of these mutations have been directly linked to dilated cardiomyopathy, hypertrophic cardiomyopathy, or arrhythmic right ventricular cardiomyopathy (Table 8.) (Landstrom et al. Am Heart J. 161:165-171 (2011), Lee et al. Cardiol Young. 24:953-954 (2014); Haghighi et al. J. Clin. Invest. 111:869-876 (2003); Schmitt et al. Science 299:1410-1413 (2003); Haghighi et al. Proc. Natd. Acad. Sci. U.S.A 103:1388-1393 (2006); Medeiros A et al. Am. Heart J. 162:1088-1095 (2011)).

TABLE 8
PLN mutations and associated heart diseases

| PLN Mutation | Associated Heart Disease |
|---|---|
| PLN promoter mutation | Dilated Cardiomyopathy; |
| | Hypertrophic Cardiomyopathy |
| PLN^{L39stop} | Dilated Cardiomyopathy; |
| | Hypertrophic Cardiomyopathy |
| R9C | Dilated Cardiomyopathy |
| R9L | Dilated Cardiomyopathy |
| R9H | Dilated Cardiomyopathy |
| PLN gene duplication | Dilated Cardiomyopathy |
| R14del | Dilated Cardiomyopathy; |
| | Arrhythmic Right Ventricular Cardiomyopathy |

The various mutations in PLN have different mechanisms of inducing a cardiomyopathy phenotype. For example, the R9C mutation indirectly blocks the phosphorylation of PLN by PKA and prevents the formation of monomeric PLN that can bind to SERCA. In some embodiments, the subject in need of treatment has a PLN promoter mutation. In some embodiments, the subject in need of treatment has a PLN^{L39stop} mutation. In some embodiments, the subject in need of treatment has a R9C mutation. In some embodiments, the subject in need of treatment has a R9L mutation. In some embodiments, the subject in need of treatment has a R9H mutation. In some embodiments, the subject in need of treatment has a PLN gene duplication. In some embodiments, the subject in need of treatment has a R14del mutation.

Mutations can be detected by many types of genetic analysis known in the art. Genetic analysis can be, for example, direct sequencing, fluorescent in situ hybridization assays, polymerase chain reaction-based assays, nucleotide microarray assays, or any other technique known in the art to determine the sequence characteristics of polynucleotides sampled from a subject. For example, DNA was isolated from the peripheral blood samples patients diagnosed with either dilated cardiomyopathy (DCM) or arrhythmic right ventricular cardiomyopathy (ARVC). The coding region of the PLN gene in the isolated DNA was sequenced using a BigDye Terminator DNA sequencing kit (version 2.0) on a 3730 Genetic Analyzer (Applied Biosystems, Foster City, CA, USA). Patients diagnosed with either DCM or ARVC both carried the PLN R14del mutation (van der Zwaag et al. Eur J Heart Fail. 14:1199-1207 (2012)).

### Methods of Reducing, Improving, and Preventing Symptoms

In some embodiments, the disclosure provides a method of reducing one or more symptoms of a heart disease or disorder in a subject comprising administration of any vector comprising an expression cassette described herein. In some embodiments, the symptoms are reduced compared to the symptoms of the heart disease or disorder prior to administration of the vector comprising an expression cassette described herein to the subject. In some embodiments, the disclosure provides a method of reducing one or more symptoms of a heart disease or disorder in a subject comprising administration of an rAAV virion described herein. In some embodiments, the symptoms are reduced compared to the symptoms of the heart disease or disorder prior to administration of the rAAV virion to the subject. In some embodiments, the heart disease or disorder is heart failure. In some embodiments, the heart disease or disorder is cardiomyopathy. In some embodiments, the heart disease or disorder is dilated cardiomyopathy. In some embodiments, the heart disease or disorder is myocardial infarction. In some embodiments, the heart disease or disorder is chronic myocardial infarction. In some embodiments, the heart disease or disorder is acute myocardial infarction.

In some embodiments, the disclosure provides a method of improving one or more symptoms of a heart disease or disorder in a subject comprising administration of a vector comprising an expression cassette described herein. In some embodiments, the symptoms are improved compared to the symptoms of the heart disease or disorder prior to administration of the vector to the subject. In some embodiments, the disclosure provides a method of improving one or more symptoms of a heart disease or disorder in a subject comprising administration of an rAAV virion described herein. In some embodiments, the symptoms are improved compared to the symptoms of the heart disease or disorder prior to administration of the rAAV virion to the subject. In some embodiments, the heart disease or disorder is heart failure. In some embodiments, the heart disease or disorder is cardiomyopathy. In some embodiments, the heart disease or disorder is dilated cardiomyopathy. In some embodiments, the heart disease or disorder is myocardial infarction. In some embodiments, the heart disease or disorder is chronic myocardial infarction. In some embodiments, the heart disease or disorder is acute myocardial infarction.

In some embodiments, the disclosure provides a method of preventing one or more symptoms of a heart disease or disorder in a subject comprising administration of a vector comprising an expression cassette described herein. In some embodiments, the disclosure provides a method of preventing one or more symptoms of a heart disease or disorder in a subject comprising administration of the rAAV virion described herein. In some embodiments, the symptoms are prevented in a subject considered to be at-risk of the heart disease or disorder. In some embodiments, the heart disease or disorder is heart failure. In some embodiments, the heart disease or disorder is cardiomyopathy. In some embodiments, the heart disease or disorder is dilated cardiomyopathy. In some embodiments, the heart disease or disorder is myocardial infarction. In some embodiments, the heart disease or disorder is chronic myocardial infarction. In some embodiments, the heart disease or disorder is acute myocardial infarction.

In some embodiments, the symptoms are reduced compared to the symptoms of the heart disease or disorder prior to administration of the vector to the subject. In some embodiments, the symptoms are reduced compared to the symptoms of the heart disease or disorder prior to administration of the rAAV virion to the subject. In some embodiments, the heart disease or disorder is heart failure. In some embodiments, the heart disease or disorder is cardiomyopathy. In some embodiments, the heart disease or disorder is dilated cardiomyopathy. In some embodiments, the heart disease or disorder is myocardial infarction. In some embodiments, the heart disease or disorder is chronic myocardial infarction. In some embodiments, the heart disease or disorder is acute myocardial infarction.

As used herein, “symptoms” include any of the diagnostic criteria or symptoms associated with, e.g., heart diseases described herein. Severity and changes of symptoms and diagnostic results are determined by a medical professional qualified to deliver assessments and analyze the results of such assessments.

Common symptoms in subjects with or at risk of developing heart disease are fatigue, dyspnea, edema, chest pain, arrhythmias, blood clots, impaired heart valve function, and heart murmur. In some embodiments, the subject experiences reduced symptoms associated with the heart diseases described herein following administration of the vector, an rAAV virion or compositions of the disclosure. In some embodiments, the improved symptoms are one or more of enhanced contractility; reduced fatigue; reduced dyspnea; reduced edema; reduced chest pain; reduced arrhythmias; reduced blood clots; improved heart valve function; and reduced heart murmur. In some embodiments, the symptom is a change in 6 minute walk distance. In some embodiments, symptoms are determined by the Minnesota Living with Heart Failure Questionnaire. In some embodiments, the symptom is an abnormal level of B-type natriuretic peptide (i.e., BNP, NT-proBNP). In some embodiments, the severity of symptoms are determined by measuring LV remodeling. In some embodiments of the method described herein improves one or more measures of cardiac function. In some embodiments, the measures of cardiac function comprise fractional shortening and/or left ventricular internal dimension (LVID). In some embodiments, the measures of cardiac function comprises left ventricular end-systolic volume (LVESV). In some embodiments, the improvement in cardiac function is ejection fraction. In some embodiments, improvement in cardiac function is observed at weeks 2 through 12. In some embodiments, the method reduces cardiac remodeling. In some embodiments, the method counteracts a decrease in DWORF expression in subjects suffering from myocardial infarction.

Ejection fraction is a measurement of the percentage of blood leaving the heart each time it contracts. The ejection fraction is determined using the stroke volume (SV) and the end-diastolic volume (EDV), calculated as: EF(%) (SV/EDV)×100. Ejection fraction can be measured in a subject with imaging tests, including echocardiogram, cardiac catheterization, magnetic resonance imaging (MRI), computerized tomography (CT), and/or nuclear medicine scan. A normal ejection fraction is between about 50% and about 75%. A “borderline” ejection fraction can range between about 41% and about 50%. A reduced ejection fraction is less than about 41%. A borderline or reduced ejection fraction can be used as a symptom in diagnosing a heart disease or disorder. It is understood that the cutoff values between normal, borderline, and reduced ejection fraction are approximate and one skilled in the art, e.g., a cardiologist, will ultimately make the determination.

In some embodiments of the methods provided herein, it may be desirable to improve ejection fraction. Ejection fraction can be considered to be improved if the ejection fraction percentage increases. In some embodiments, the ejection fraction

In some embodiments of the methods provided herein, it may be desirable to preserve ejection fraction. Preserving ejection fraction can be used to prevent the onset of a heart disease or disorder in a subject at risk thereof, prevent the progression of a heart disease or disorder, or prevent worsening of symptoms associated with a heart disease or disorder in a subject at risk of or suffering therefrom.

The disclosure provides methods of improving ejection fraction in a subject at risk or suffering from a heart disease or disorder. In some embodiments, ejection fraction is improved (i.e., increased) in the subject following administration of a vector comprising an expression cassette described herein. In some embodiments, ejection fraction is improved (i.e., increased) in the subject following administration of a vector or an rAAV virion described herein. In some embodiments, ejection fraction is improved about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 22 weeks or about 24 weeks following administration of the vector or the rAAV virion to the subject. In some embodiments, ejection fraction is improved about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% following administration of the vector or the rAAV virion to the subject.

The disclosure provides methods of preserving ejection fraction in a subject at risk or suffering from a heart disease or disorder. For example, the subject may maintain an ejection fraction that would otherwise be expected to reduce in the absence of administration of the vector or the rAAV virion or pharmaceutical composition of the disclosure. In some embodiments, ejection fraction is preserved in the subject following administration of the vector or the rAAV virion of the disclosure. In some embodiments, ejection fraction is preserved about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 22 weeks or about 24 weeks following administration of the vector or rAAV virion to the subject. In some embodiments, ejection fraction is preserved by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% following administration of the vector or rAAV virion to the subject.

Assessment of heart contractility can be used to assess acute and chronic forms of heart failure. Heart contractility may be monitored by using invasive hemodynamic monitoring, continuous ECG monitoring, central venous pressure, kidney function, pulse oximetry, arterial pressure monitoring, pulmonary artery catheter, and/or transeophageal echocardiography (Kuhn C, Werdan K. Surgical Treatment: Evidence-Based and Problem-Oriented. Munich: Zuckschwerdt; 2001. Available from: https://www.ncbi.nlm.nih.gov/books/NBK6895/).

Dyspnea and fatigue associated with heart disease described herein can be measured using questionnaires. The Modified Pulmonary Functional Status and Dyspnea Questionnaire (PFSDQ-M)10 (Huang et al. Am J Crit Care. 17:436-442 (2008)) and Minnesota Living with Heart Failure Questionnaire (MLHFQ)11 (Bilbao et al. Health Qual Life Outcomes. 14:23 (2016)), for example, can be used to measure subjects with a heart disease as described herein. The questionnaires are self-administered and allow a score to be derived that is used to assess symptom severity for dyspnea, fatigue, and other heart-health related symptoms.

Cardiomyopathy, myocardial infarction and heart valve function may be assessed using one or more of an exercise stress test, electrocardiogram, echocardiogram, chest X-ray, cardiac CT scan, or angiogram with cardiac catheterization, cardiac MRI, B-type natriuretic peptide (BNP) levels in the blood, and/or genetic screening. Further testing is required to diagnose specific types of cardiomyopathy, myocardial infarction, or heart valve dysfunction.

In some aspects, administration of a vector comprising an expression cassette described herein to a subject results in an improvement in exercise capacity of the subject (e.g., improvement in running distance and/or time to exhaustion). In some aspects, administration of an rAAV comprising an expression cassette described herein encoding DWORF to a subject results in an improvement in exercise capacity of the subject (e.g., improvement in running distance and/or time to exhaustion).

Dilated cardiomyopathy (DCM) is a progressive disease of heart muscle characterized by chamber enlargement and contractile dysfunction of the left ventricle in the absence of chronic pressure and/or volume overload. DCM is diagnosed primarily using echocardiography.

Echocardiography with a PLAX view in 2D/M-mode is used to measure several parameters, including ejection fraction, LVIDd/s, IVSd, LVPWd, and fractional shortening. These parameters are used to assess the left ventricle cavity size, wall thickness, and radial function. Diagnostic criterion for DCM includes LVIDd/s greater than 112% (2 S.D) corrected for age and body surface area (BSA). Fractional shortening less than 25% is a criterion for the diagnosis of DCM in the presence of a dilated ventricle (Mathew et al. Echo Res Pract. 4:G1-G13 (2017)).

Qualitative assessment of left and right ventricular structure and function with special reference to radial and longitudinal function and regional wall motion abnormalities are assessed by echocardiography in the apical four-chamber (A4C) view in 2D mode. Ejection fraction (EF) can be estimated using, for example, biplane Simpsons method. EF of less than 45% is a diagnostic criterion for DCM in the presence of dilated ventricle (Mathew et al. Echo Res Pract. 4: G1-G13 (2017)).

### Administration

In some embodiments, the vectors and compositions of the present disclosure can be administered to a subject in need thereof by systemic application, e.g., by intravenous, intra-arterial or intraperitoneal delivery. In some embodiments, the rAAV virion and compositions of the present disclosure can be administered to a subject in need thereof by systemic application, e.g., by intravenous, intra-arterial or intraperitoneal delivery of a vector in analogy to what has been shown in animal models (Katz et al., Gene Ther 19:659-669 (2012)). In some embodiments, the vectors, rAAV virions and compositions of the present disclosure treat or prevent heart failure. In some embodiments, the cardiomyopathy, wherein the vector is administered systemically. In some embodiments, the rAAV virion is administered by intravenous or intracoronary injection.

The disclosure provides methods for expressing a polypeptide in a cell in vitro, ex vivo, or in vivo. In some embodiments, the disclosure provides methods for expressing a DWORF polypeptide in a cell in vitro, ex vivo, or in vivo. The method comprises, for example, exposing a target cell to the vectors, rAAV virions or pharmaceutical compositions described herein. A target cell can be, for example and without limitation, a cardiac cell, a muscle cell, an induced pluripotent stem cell-derived cardiomyocyte (iPSC-CM), and/or a cardiomyocyte. In some embodiments, a method of expressing a polypeptide (e.g., DWORF polypeptide) in a cell comprises transfecting or transducing (alternating referred to as “infecting”) a target cell or population of target cells with a vector described herein. In some embodiments, a method of expressing a polypeptide (e.g., DWORF polypeptide) in a cell comprises transducing (alternating referred to as “infecting”) a target cell or population of target cells with an rAAV virion or pharmaceutical compositions described herein. In some embodiments, the rAAV transduces cardiac cells. In some embodiments, the rAAV transduces cardiomyocytes. In some embodiments, the rAAV transduces induced pluripotent stem cell-derived cardiomyocytes (iPSC-CM).

In some embodiments, the vector transfection or transduction increases polypeptide expression in the heart of the subject. In some embodiments, the rAAV transduction increases DWORF polypeptide expression in the heart of the subject. “Increased polypeptide expression” typically refers to expression at least 5%, 10%, 15%, 20% or more compared to a control subject or tissue not treated with the vector. “Increased DWORF polypeptide expression” typically refers to expression at least 5%, 10%, 15%, 20% or more compared to a control subject or tissue not treated with the vector. In some embodiments, detectable expression means expression at 1.5-fold, 2-fold, 2.5-fold, or 3-fold greater than a no-vector control. Expression can be assessed by Western blot, as described in the example that follows, or enzyme-linked immunosorbent assay (ELISA), or other methods known in the art. In some cases, expression is measured quantitatively using a standard curve. Standard curves can be generated using purified protein, e.g., purified DWORF polypeptide, by methods described in the examples or known in the art. Alternatively, expression of the therapeutic gene product can be assessed by quantification of the corresponding mRNA. In some embodiments, the method causes the expression of the polypeptide (e.g., DWORF polypeptide) in the heart of the subject.

In some embodiments, the method causes no detectable expression of the polypeptide in the muscles of the subject except the heart, in the liver of the subject, and/or in cardiac fibroblasts. In some embodiments, the method causes expression of the polypeptide in cardiomyocytes.

In some embodiments, the method causes no detectable expression of the DWORF polypeptide in the muscles of the subject except the heart. In some embodiments, the method causes no detectable expression of the DWORF polypeptide in the liver of the subject. In some embodiments, the method causes expression of the DWORF polypeptide in cardiomyocytes. In some embodiments, the method causes no detectable expression of the DWORF polypeptide in cardiac fibroblasts.

In some embodiments, the increased polypeptide expression in heart tissue occurs at doses, in vector genomes (vg) per kilogram weight of subject (kg), of 3×10¹⁴ vg/kg or less, 2×10¹⁴ vg/kg or less, 1×10¹⁴ vg/kg or less, 9×10¹³ vg/kg or less, 8×10¹³ vg/kg or less, 7×10¹³ vg/kg or less, 6×10¹³ vg/kg or less, 5×10¹³ vg/kg or less, 4×10¹³ vg/kg or less, 3×10¹³ vg/kg or less, 2×10¹³ vg/kg or less, or 1×10¹³ vg/kg or less.

In some embodiments, the increased DWORF expression in heart tissue occurs at doses, in vector genomes (vg) per kilogram weight of subject (kg), of 3×10¹⁴ vg/kg or less, 2×10¹⁴ vg/kg or less, 1×10¹⁴ vg/kg or less, 9×10¹³ vg/kg or less, 8×10¹³ vg/kg or less, 7×10¹³ vg/kg or less, 6×10¹³ vg/kg or less, 5×10¹³ vg/kg or less, 4×10¹³ vg/kg or less, 3×10¹³ vg/kg or less, 2×10¹³ vg/kg or less, or 1×10¹³ vg/kg or less.

### Pharmaceutical Compositions and Kits

The vectors of the disclosure are generally delivered to the subject as a pharmaceutical composition. In some embodiments, the rAAV virion of the disclosure is delivered to the subject as a pharmaceutical composition. Pharmaceutical compositions comprise a pharmaceutically acceptable solvent (e.g., water, etc.) and one or more excipients. In some embodiments, the pharmaceutical compositions comprise a buffer at about neutral pH (pH 5, 6, 7, 8, or 9). In some embodiments, the pharmaceutical composition comprises phosphate buffered saline (e.g., PBS at pH of about 7). The pharmaceutical compositions may comprise a pharmaceutically acceptable salt. The concentration of the salt may be selected to ensure that the pharmaceutical composition is isotonic to, or nearly isotonic to, the target tissue.

In various embodiments, the compositions described herein contain vehicles (e.g., carriers, diluents and excipients) that are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. Illustrative pharmaceutical forms suitable for injectable use include, e.g., sterile aqueous solutions or dispersions; formulations including sesame oil, peanut oil or aqueous propylene glycol; and sterile powders for the extemporaneous preparation of sterile injectable solutions or dispersions.

In various embodiments, the pharmaceutical compositions of the disclosure comprise about 1×10⁸ genome copies per milliliter (GC/mL), about 5×10⁸ GC/mL, about 1×10⁹ GC/mL, about 5×10⁹ GC/mL, about 1×10¹⁰ GC/mL, about 5×10¹⁰ GC/mL, about 1×10¹¹ GC/mL, about 5×10¹¹ GC/mL, about 1×10¹² GC/mL, about 5×10¹² GC/mL, about 5×10¹³ GC/mL, about 1×10¹⁴ GC/mL, or about 5×10¹⁴ GC/mL of the viral vector (e.g., rAAV virion).

In various embodiments, the pharmaceutical compositions of the disclosure comprise about 1×10⁸ viral genomes per milliliter (vg/mL), about 5×10⁸ vg/mL, about 1×10⁹ vg/mL, about 5×10⁹ vg/mL, about 1×10¹⁰ vg/mL, about 5×10¹⁰ vg/mL, about 1×10¹¹ vg/mL, about 5×10¹¹ vg/mL, about 1×10¹² vg/mL, about 5×10¹² vg/mL, about 5×10¹³ vg/mL, about 1×10¹⁴ vg/mL, or about 5×10¹⁴ vg/mL of the viral vector (e.g., rAAV virion).

In some embodiments, the pharmaceutical compositions of the disclosure are administered in a total volume of about 1 mL, 5 mL, 10 mL, about 20 mL, about 25 mL, about 30 mL, about 35 mL, about 40 mL, about 45 mL, about 50 mL, about 55 mL, about 60 mL, 65 mL, about 70 mL, about 75 mL, about 80 mL, about 85 mL, about 90 mL, about 95 mL, about 100 mL, about 105 mL, about 110 mL, about 115 mL, about 120 mL, about 125 mL, about 130 mL, about 135 mL, about 140 mL, about 145 mL, about 150 mL, about 155 mL, about 160 mL, about 165 mL, about 170 mL, about 175 mL, about 180 mL, about 185 mL, about 190 mL, about 200 mL, about 205 mL, about 210 mL, about 215 mL, or about 220 mL.

Genome copies per milliliter can be determined by quantitative polymerase change reaction (qPCR) using a standard curve generated with a reference sample having a known concentration of the polynucleotide genome of the virus. For AAV, the reference sample used is often the transfer plasmid used in generation of the rAAV virion but other reference samples may be used.

Alternatively or in addition, the concentration of a viral vector can be determined by measuring the titer of the vector on a cell line. Viral titer is typically expressed as viral particles (vp) per unit volume (e.g., vp/mL). In various embodiments, the pharmaceutical compositions of the disclosure comprise about 1×10⁸ viral particles per milliliter (vp/mL), about 5×10⁸ vp/mL, about 1×10⁹ vp/mL, about 5×10⁹ vp/mL, about 1×10¹⁰ vp/mL, about 5×10¹⁰ vp/mL, about 1×10¹¹ vp/mL, about 5×10¹¹ vp/mL, about 1×10¹² vp/mL, about 5×10¹² vp/mL, about 5×10¹³ vp/mL, or about 1×10¹⁴ vp/mL, or about 5×10¹⁴ of the viral vector (e.g., rAAV virion).

In some embodiments, the present disclosure provides a kit comprising a container housing a pharmaceutical composition as described herein.

### Numbered Embodiments of the Invention I

Embodiment 1: A recombinant adeno-associated virus (rAAV) virion, comprising a capsid protein and a viral genome comprising an expression cassette comprising a polynucleotide sequence encoding a dwarf open reading frame (DWORF) polypeptide operatively linked to a promoter, the expression cassette flanked by inverted terminal repeats.

Embodiment 2: The rAAV virion of embodiment 1, wherein the DWORF polypeptide shares at least 90%, 95%, 96%, 97%, 98%, or 99% identity to a sequence selected from SEQ ID NOs: 1, 3, 4, 7, 9, 23, and 43.

Embodiment 3: The rAAV virion of embodiment 1, wherein the DWORF polypeptide is selected from SEQ ID NOs: 1, 3, 4, 7, 9, 23, and 43.

Embodiment 4: The rAAV virion of embodiment 1 or 2, wherein the promoter is a chicken cTnT promoter.

Embodiment 5: The rAAV virion of embodiment 4, wherein the chicken cTnT promoter shares at least 90%, 95%, 96%, 97%, 98%, or 99% identity to SEQ ID NO: 11.

Embodiment 6: The rAAV virion of embodiment 4, wherein the chicken cTnT promoter comprises SEQ ID NO: 11.

Embodiment 7: The rAAV virion of embodiment 1 or 2, wherein the promoter is a human cTnT promoter.

Embodiment 8: The rAAV virion of embodiment 7, wherein the human cTnT promoter shares at least 90%, 95%, 96%, 97%, 98%, or 99% identity to SEQ ID NO: 12 or SEQ ID NO: 13.

Embodiment 9: The rAAV virion of embodiment 7, wherein the human cTnT promoter comprises SEQ ID NO: 12 or SEQ ID NO: 13.

Embodiment 10: The rAAV virion of any one of embodiments 1 to 9, wherein the expression cassette further comprises one or more enhancers.

Embodiment 11: The rAAV virion of embodiment 10, wherein the enhancer the one or more enhancers are selected from a ACTC1 cardiac enhancer and a αMHC enhancer.

Embodiment 12: The rAAV virion of embodiment 11, wherein the ACTC1 cardiac enhancer shares at least 90%, 95%, 96%, 97%, 98%, or 99% identity to SEQ ID NO: 78.

Embodiment 13: The rAAV virion of embodiment 11, wherein the ACTC1 cardiac enhancer comprises SEQ ID NO: 78.

Embodiment 14: The rAAV virion of embodiment 11, wherein the αMHC enhancer shares at least 90%, 95%, 96%, 97%, 98%, or 99% identity to SEQ ID NO: 79.

Embodiment 15: The rAAV virion of embodiment 11, wherein the αMHC enhancer comprises SEQ ID NO: 79.

Embodiment 16: The rAAV virion of any one of embodiments 1 to 15, wherein the expression cassette further comprises an intron.

Embodiment 17: The rAAV virion of embodiment 16, wherein the intron is selected from a CMV intron and a chimeric intron.

Embodiment 18: The rAAV virion of embodiment 17, wherein the CMV intron shares at least 90%, 95%, 96%, 97%, 98%, or 99% identity to SEQ ID NO: 80.

Embodiment 19: The rAAV virion of embodiment 17, wherein the CMV intron comprises SEQ ID NO: 80.

Embodiment 20: The rAAV virion of embodiment 17, wherein the chimeric intron shares at least 90%, 95%, 96%, 97%, 98%, or 99% identity to SEQ ID NO: 81.

Embodiment 21: The rAAV virion of embodiment 17, wherein the chimeric intron comprises SEQ ID NO: 81.

Embodiment 22: The rAAV virion of any one of embodiments 1 to 21, wherein the expression cassette further comprises a WPRE sequence.

Embodiment 23: The rAAV virion of embodiment 22, wherein the WPRE sequence shares at least 90%, 95%, 96%, 97%, 98%, or 99% identity to SEQ ID NO: 26.

Embodiment 24: The rAAV virion of embodiment 22, wherein the WPRE sequence comprises SEQ ID NO: 26.

Embodiment 25: The rAAV virion of any one of embodiments 1 to 24, wherein the expression cassette further comprises a polyadenylation sequence.

Embodiment 26: The rAAV virion of embodiment 25, wherein the polyadenylation sequence is selected from a BGH polyadenylation sequence and a SV40 polyadenylation sequence.

Embodiment 27: The rAAV virion of embodiment 26, wherein the BGH polyadenylation sequence shares at least 90%, 95%, 96%, 97%, 98%, or 99% identity to SEQ ID NO: 27.

Embodiment 28: The rAAV virion of embodiment 26, wherein the BGH polyadenylation sequence comprises SEQ ID NO: 27.

Embodiment 29: The rAAV virion of embodiment 26, wherein the SV40 polyadenylation sequence shares at least 90%, 95%, 96%, 97%, 98%, or 99% identity to SEQ ID NO: 28.

Embodiment 30: The rAAV virion of embodiment 26, wherein the SV40 polyadenylation sequence comprises SEQ ID NO: 28.

Embodiment 31: The rAAV virion of any one of embodiments 1 to 30, wherein the expression cassette is flanked by ITRs.

Embodiment 32: The rAAV virion of embodiment 31, wherein the ITRs share at least 90%, 95%, 96%, 97%, 98%, or 99% identity to one or more of SEQ ID NO: 14 and SEQ ID NO: 15.

Embodiment 33: The rAAV virion of embodiment 31, wherein the ITRs comprise one or more of SEQ ID NO: 14 and SEQ ID NO: 15.

Embodiment 34: The rAAV virion of any one of embodiments 1 to 33, wherein the expression cassette comprises a single promoter.

Embodiment 35: The rAAV virion of any one of embodiments 1 to 33, wherein the expression cassette comprises two promoters.

Embodiment 36: The rAAV virion of any one of embodiments 1 to 35 wherein the expression cassette comprises a single copy a sequence encoding the DWORF polypeptide.

Embodiment 37: The rAAV virion of any one of embodiments 1 to 35 wherein the expression cassette comprises two copies of a sequence encoding the DWORF polypeptide.

Embodiment 38: The rAAV virion of any one of embodiments 1 to 37 wherein the expression cassette comprises one, two, three, or four enhancers.

Embodiment 39: The rAAV virion of any one of embodiments 1 to 38 wherein the expression cassette comprises one or two introns.

Embodiment 40: The rAAV virion of any one of embodiments 1 to 39 wherein the expression cassette comprises one or two WPRE sequences.

Embodiment 41: The rAAV virion of any one of embodiments 1 to 40 wherein the expression cassette comprises one or two polyadenylation sequences.

Embodiment 42: The rAAV virion of any one of embodiments 1 to 41 wherein the expression cassette comprises about 3.2 kb, about, about 3.3 kb, about 3.4 kb, about 3.5 kb, about 3.6 kb, about 3.7 kb, or less.

Embodiment 43: The rAAV virion of any one of embodiments 1 to 41 wherein the expression cassette comprises about 1.9 kb, about 2.1 kb, about 2.2 kb, about 2.3 kb, about 2.4 kb, about 2.5 kb, about 2.6 kb, about 2.7 kb, about 2.8 kb, about 2.9 kb, about 3.0 kb, about 3.1 kb, about 3.2 kb, or more.

Embodiment 44: The rAAV virion of embodiment 1, wherein the expression cassette comprises a polynucleotide sequence that shares at least 75%, 80%, 90%, 95%, 96%, 97%, 98%, 99%, or 100% identity to any one of SEQ ID NOs: 20-24 or SEQ ID NOs: 45-75.

Embodiment 45: The rAAV virion of embodiment 1, wherein the expression cassette comprises any one of SEQ ID NOs: 20-24 or SEQ ID NOs: 45-75.

Embodiment 46: The rAAV virion of embodiment 1, wherein the expression cassette comprises a polynucleotide sequence that shares at least 75%, 80%, 90%, 95%, 96%, 97%, 98%, or 99% identity to SEQ ID NO: 61.

Embodiment 47: The rAAV virion of embodiment 1, wherein the expression cassette comprises SEQ ID NO: 61.

Embodiment 48: The rAAV virion of embodiment 1, wherein the expression cassette comprises a polynucleotide sequence that shares at least 75%, 80%, 90%, 95%, 96%, 97%, 98%, or 99% identity to SEQ ID NO: 62.

Embodiment 49: The rAAV virion of embodiment 1, wherein the expression cassette comprises SEQ ID NO: 62.

Embodiment 50: The rAAV virion of embodiment 1, wherein the expression cassette comprises a polynucleotide sequence that shares at least 75%, 80%, 90%, 95%, 96%, 97%, 98%, or 99% identity to SEQ ID NO: 63.

Embodiment 51: The rAAV virion of embodiment 1, wherein the expression cassette comprises SEQ ID NO: 63.

Embodiment 52: The rAAV virion of any one of embodiments 1 to 51, wherein the capsid protein shares at least 98%, at least 99%, or 100% identity to an AAV9 capsid protein (SEQ ID NO: 143).

Embodiment 53: The rAAV virion of any one of embodiments 1 to 51, wherein the capsid protein shares at least 98%, at least 99%, or 100% identity to an AAV5 capsid protein (SEQ ID NO: 144).

Embodiment 54: The rAAV virion of any one of embodiments 1 to 51, wherein the capsid protein is a chimeric capsid protein.

Embodiment 55: The rAAV virion of any one of embodiments 1 to 51, wherein the capsid protein is an AAV5/AAV9 chimeric capsid protein.

Embodiment 56: The rAAV virion of any one of embodiments 1 to 51, wherein the capsid protein is selected from any one of SEQ ID NOs: 145-200.

Embodiment 57: An expression cassette comprising polynucleotide sequence encoding a dwarf open reading frame (DWORF) polypeptide operatively linked to a promoter.

Embodiment 58: The expression cassette of embodiment 57, wherein the DWORF polypeptide is selected from SEQ ID NOs: 1, 3, 4, 7, 9, 23, and 43.

Embodiment 59: The expression cassette of embodiment 57 or 58, wherein the promoter is a chicken cTnT promoter.

Embodiment 60: The expression cassette of embodiment 59, wherein the chicken cTnT promoter shares at least 90%, 95%, 96%, 97%, 98%, or 99% identity to SEQ ID NO: 11.

Embodiment 61: The expression cassette of embodiment 59, wherein the chicken cTnT promoter comprises SEQ ID NO: 11.

Embodiment 62: The expression cassette of embodiment 57 or 58, wherein the promoter is a human cTnT promoter.

Embodiment 63: The expression cassette of embodiment 62, wherein the human cTnT promoter shares at least 90%, 95%, 96%, 97%, 98%, or 99% identity to SEQ ID NO: 12 or SEQ ID NO: 13.

Embodiment 64: The expression cassette of embodiment 62, wherein the human cTnT promoter comprises SEQ ID NO: 12 or SEQ ID NO: 13.

Embodiment 65: The expression cassette of any one of embodiments 57 to 64, wherein the expression cassette further comprises one or more enhancers.

Embodiment 66: The expression cassette of embodiment 65, wherein the enhancer the one or more enhancers are selected from a ACTC1 cardiac enhancer and a αMHC enhancer.

Embodiment 67: The expression cassette of embodiment 66, wherein the ACTC1 cardiac enhancer shares at least 90%, 95%, 96%, 97%, 98%, or 99% identity to SEQ ID NO: 78.

Embodiment 68: The expression cassette of embodiment 66, wherein the ACTC1 cardiac enhancer comprises SEQ ID NO: 78.

Embodiment 69: The expression cassette of embodiment 66, wherein the αMHC enhancer shares at least 90%, 95%, 96%, 97%, 98%, or 99% identity to SEQ ID NO: 79.

Embodiment 70: The expression cassette of embodiment 66, wherein the αMHC enhancer comprises SEQ ID NO: 79.

Embodiment 71: The expression cassette of any one of embodiments 57 to 70, wherein the expression cassette further comprises an intron.

Embodiment 72: The expression cassette of any one of embodiments 57 to 70, wherein the intron is selected from a CMV intron and a chimeric intron.

Embodiment 73: The expression cassette of embodiment 72, wherein the CMV intron shares at least 90%, 95%, 96%, 97%, 98%, or 99% identity to SEQ ID NO: 80.

Embodiment 74: The expression cassette of embodiment 72, wherein the CMV intron comprises SEQ ID NO: 80.

Embodiment 75: The expression cassette of embodiment 72, wherein the chimeric intron shares at least 90%, 95%, 96%, 97%, 98%, or 99% identity to SEQ ID NO: 81.

Embodiment 76: The expression cassette of embodiment 72, wherein the chimeric intron comprises SEQ ID NO: 81.

Embodiment 77: The expression cassette of any one of embodiments 57 to 76, wherein the expression cassette further comprises a WPRE sequence.

Embodiment 78: The expression cassette of embodiment 77, wherein the WPRE sequence shares at least 90%, 95%, 96%, 97%, 98%, or 99% identity to SEQ ID NO: 26.

Embodiment 79: The expression cassette of embodiment 77, wherein the WPRE sequence comprises SEQ ID NO: 26.

Embodiment 80: The expression cassette of any one of embodiments 57 to 79, wherein the expression cassette further comprises a polyadenylation sequence.

Embodiment 81: The expression cassette of embodiment 80, wherein the polyadenylation sequence is selected from a BGH polyadenylation sequence and a SV40 polyadenylation sequence.

Embodiment 82: The expression cassette of embodiment 81, wherein the BGH polyadenylation sequence shares at least 90%, 95%, 96%, 97%, 98%, or 99% identity to SEQ ID NO: 27.

Embodiment 83: The expression cassette of embodiment 81, wherein the BGH polyadenylation sequence comprises SEQ ID NO: 27.

Embodiment 84: The expression cassette of embodiment 81, wherein the SV40 polyadenylation sequence shares at least 90%, 95%, 96%, 97%, 98%, or 99% identity to SEQ ID NO: 28.

Embodiment 85: The expression cassette of embodiment 81, wherein the SV40 polyadenylation sequence comprises SEQ ID NO: 28.

Embodiment 86: The expression cassette of any one of embodiments 57 to 85, wherein the expression cassette is flanked by ITRs.

Embodiment 87: The expression cassette of embodiment 86, wherein the ITRs share at least 90%, 95%, 96%, 97%, 98%, or 99% identity to one or more of SEQ ID NO: 14 and SEQ ID NO: 15.

Embodiment 88: The expression cassette of embodiment 86, wherein the ITRs comprise one or more of SEQ ID NO: 14 and SEQ ID NO: 15.

Embodiment 89: The expression cassette of any one of embodiments 57 to 88 comprising a single promoter.

Embodiment 90: The expression cassette of any one of embodiments 57 to 88 comprising two promoters.

Embodiment 91: The expression cassette of any one of embodiments 57 to 90 comprising a single copy a sequence encoding the DWORF polypeptide.

Embodiment 92: The expression cassette of any one of embodiments 57 to 90 comprising two copies of a sequence encoding the DWORF polypeptide.

Embodiment 93: The expression cassette of any one of embodiments 57 to 92 comprising one, two, three, or four enhancers.

Embodiment 94: The expression cassette of any one of embodiments 57 to 93 comprising one or two introns.

Embodiment 95: The expression cassette of any one of embodiments 57 to 94 comprising one or two WPRE sequences.

Embodiment 96: The expression cassette of any one of embodiments 57 to 95 comprising one or two polyadenylation sequences.

Embodiment 97: The expression cassette of any one of embodiments 57 to 96 comprising about 3.2 kb, about, about 3.3 kb, about 3.4 kb, about 3.5 kb, about 3.6 kb, about 3.7 kb, or less.

Embodiment 98: The expression cassette of any one of embodiments 57 to 96 comprising about 1.9 kb, about 2.1 kb, about 2.2 kb, about 2.3 kb, about 2.4 kb, about 2.5 kb, about 2.6 kb, about 2.7 kb, about 2.8 kb, about 2.9 kb, about 3.0 kb, about 3.1 kb, about 3.2 kb, or more.

Embodiment 99: The expression cassette of embodiment 57, comprising a polynucleotide sequence that shares at least 75%, 80%, 90%, 95%, 96%, 97%, 98%, 99%, or 100% identity to any one of SEQ ID NOs: 20-24 or SEQ ID NOs: 45-75.

Embodiment 100: The expression cassette of embodiment 57, comprising any one of SEQ ID NOs: 20-24 or SEQ ID NOs: 45-75.

Embodiment 101: The expression cassette of embodiment 57, comprising a polynucleotide sequence that shares at least 75%, 80%, 90%, 95%, 96%, 97%, 98%, or 99% identity to SEQ ID NO: 61.

Embodiment 102: The expression cassette of embodiment 57, comprising SEQ ID NO: 61.

Embodiment 103: The expression cassette of embodiment 57, comprising a polynucleotide sequence that shares at least 75%, 80%, 90%, 95%, 96%, 97%, 98%, or 99% identity to SEQ ID NO: 62.

Embodiment 104: The expression cassette of embodiment 57, comprising SEQ ID NO: 62.

Embodiment 105: The expression cassette of embodiment 57, comprising a polynucleotide sequence that shares at least 75%, 80%, 90%, 95%, 96%, 97%, 98%, or 99% identity to SEQ ID NO: 63.

Embodiment 106: The expression cassette of embodiment 57, comprising SEQ ID NO: 63.

Embodiment 107: The expression cassette of any one of embodiments 57 to 98, further comprising a 5′ inverted terminal repeat and a 3′ inverted terminal repeat.

Embodiment 108: A pharmaceutical composition comprising the rAAV virion of any one of embodiments 1 to 56 and an pharmaceutically acceptable diluent.

Embodiment 109: A kit comprising the pharmaceutical composition of embodiment 108.

Embodiment 110: A method of increasing DWORF expression in a cell comprising contacting a cell with the rAAV virion of any one of embodiments 1 to 56 or the composition of embodiment 108.

Embodiment 111: The method of embodiment 110, wherein the cell is a cardiac cell.

Embodiment 112: The method of embodiment 111, wherein the cardiac cell is a cardiomyocyte.

Embodiment 113: The method of any one of embodiments 110 to 112, wherein DWORF expression is increased between about 1.5-fold and 150-fold.

Embodiment 114: The method of any one of embodiments 110 to 113, wherein the contacting is in vitro.

Embodiment 115: The method of any one of embodiments 110 to 113, wherein the contacting is in vivo.

Embodiment 116: A method of increasing DWORF expression in a tissue comprising contacting the tissue with the rAAV virion of any one of embodiments 1 to 56 or the composition of embodiment 108.

Embodiment 117: The method of embodiment 116, wherein the tissue is cardiac tissue.

Embodiment 118: The method of embodiment 116 or 117, wherein DWORF expression is increased between about 1.5-fold and 150-fold.

Embodiment 119: The method of any one of embodiments 116 to 118, wherein the contacting is in vitro.

Embodiment 120: The method of embodiment 116 or 118, wherein the contacting is in vivo.

Embodiment 121: A method of increasing DWORF expression in an organ comprising contacting the organ with the rAAV virion of any one of embodiments 1 to 56 or the composition of embodiment 108.

Embodiment 122: The method of embodiment 121, wherein the organ is a heart.

Embodiment 123: The method of embodiment 122, wherein the heart is diseased or is at risk of disease.

Embodiment 124: The method of embodiment 122 or embodiment 123, wherein the heart has reduced or borderline ejection fraction.

Embodiment 125: The method of embodiment 122 or embodiment 123, wherein the heart has a normal ejection fraction.

Embodiment 126: The method of any one of embodiments 122 to 125, wherein the heart comprises a genetic mutation associated with a heart disease.

Embodiment 127: The method of embodiment 126, wherein the genetic mutation is a PLN mutation.

Embodiment 128: The method of any one of embodiments 121 to 127, wherein the heart has low or undetectable DWORF expression compared to a healthy heart.

Embodiment 129: The method of any one of embodiments 121 to 128, wherein DWORF expression is increased between about 1.5-fold and 150-fold.

Embodiment 130: The method of any one of embodiments 121 to 129, wherein the contacting is in vitro.

Embodiment 131: The method of any one of embodiments 121 to 129, wherein the contacting is in vivo.

Embodiment 132: A method of increasing DWORF expression in an subject comprising administering to the subject the rAAV virion of any one of embodiments 1 to 56 or the composition of embodiment 108.

Embodiment 133: The method of embodiment 132, wherein the subject is an animal.

Embodiment 134: The method of embodiment 132, wherein the subject is a human.

Embodiment 135: The method of any one of embodiments 132 to 134, wherein DWORF expression is increased in the heart of the subject.

Embodiment 136: The method of any one of embodiments 132 to 135, wherein subject has a heart disease or is at risk of a heart disease.

Embodiment 137: The method of any one of embodiments 132 to 136, wherein subject has borderline or reduced ejection fraction.

Embodiment 138: The method of any one of embodiments 132 to 136, wherein the subject has normal ejection fraction.

Embodiment 139: The method of any one of embodiments 132 to 138, wherein the subject has a genetic mutation associated with a heart disease.

Embodiment 140: The method of embodiment 139, wherein the genetic mutation is a PLN mutation.

Embodiment 141: The method of any one of embodiments 132 to 140, wherein the subject has a low or undetectable level of DWORF expression compared to a healthy subject.

Embodiment 142: A method of treating a heart disease or disorder in a subject in need thereof comprising administering to the subject the rAAV virion of any one of embodiments 1 to 56 or the composition of embodiment 108.

Embodiment 143: The method of embodiment 142, wherein the subject has a heart disease or disorder.

Embodiment 144: The method of embodiment 142, wherein the subject is a risk of developing a heart disease or disorder.

Embodiment 145: The method or any one of embodiments 142 to 144, wherein the heart disease or disorder is cardiomyopathy.

Embodiment 146: The method of any one of embodiments 142 to 144, wherein the heart disease or disorder is dilated cardiomyopathy.

Embodiment 147: The method of any one of embodiments 142 to 144, wherein the heart disease or disorder is myocardial infarction.

Embodiment 148: The method of any one of embodiments 142 to 144, wherein the heart disease or disorder is chronic myocardial infarction.

Embodiment 149: The method of any one of embodiments 142 to 144, wherein the heart disease or disorder is acute myocardial infarction.

Embodiment 150: The method of any one of embodiments 142 to 149, wherein the subject has an inherited risk allele for a heart disease or disorder.

Embodiment 151: The method of any one of embodiments 142 to 150, wherein the inherited risk allele comprises a mutation to the PLN gene.

Embodiment 152: The method of embodiment 151, wherein the mutation to the PLN gene is a PLN promoter mutation.

Embodiment 153: The method of embodiment 151, wherein the mutation to the PLN gene is a PLN^{L39stop} mutation.

Embodiment 154: The method of embodiment 151, wherein the mutation to the PLN gene is a RC9 mutation.

Embodiment 155: The method of embodiment 151, wherein the mutation to the PLN gene is a R9L mutation.

Embodiment 156: The method of embodiment 151, wherein the mutation to the PLN gene is a PLN gene duplication.

Embodiment 157: The method of embodiment 151, wherein the mutation to the PLN gene is a R14del mutation.

Embodiment 158: The method of any one of embodiments 142 to 157, wherein the heart disease or disorder is with reduced ejection fraction (HFrEF).

Embodiment 159: The method of any one of embodiments 142 to 157, wherein the heart disease of disorder is with preserved ejection fraction (HFpEF).

Embodiment 160: The method of any one of embodiments 142 to 159, wherein the method causes expression of the DWORF polypeptide in the heart of the subject.

Embodiment 161: The method of any one of embodiments 142 to 160, wherein the method causes expression of the DWORF polypeptide in cardiomyocytes.

Embodiment 162: The method of any one of embodiments 142 to 161, wherein the method causes no detectable expression of the DWORF polypeptide in the muscles of the subject except the heart.

Embodiment 163: The method of any one of embodiments 142 to 162, wherein the method causes no detectable expression of the DWORF polypeptide in the liver of the subject.

Embodiment 164: The method of any one of embodiments 142 to 163, wherein the method causes no detectable expression of the DWORF polypeptide in cardiac fibroblasts.

Embodiment 165: The method of any one of embodiments 142 to 164, wherein the method improves one or more measures of cardiac function, optionally fraction shortening and/or left ventricular internal dimension (LVID).

Embodiment 166: The method of any one of embodiments 142 to 165, wherein the improvement in cardiac function is observed at weeks 2 through week 16.

Embodiment 167: The method of any one of embodiments 142 to 166, wherein the method reduces cardiac remodeling.

Embodiment 168: The method of any one of embodiments 142 to 166, wherein the method counteracts a decrease in DWORF expression in subjects suffering from or at risk of a heart disease.

Embodiment 169: The method of any one of embodiments 142 to 168, wherein the rAAV virion is administered by systemic administration.

Embodiment 170: The method of embodiment 169, wherein the systemic administration is selected from intravenous or intracoronary injection.

Embodiment 171: The method of embodiment 169 or 170, wherein the rAAV is administered as a unit dose.

Embodiment 172: The method of embodiment 171, wherein the unit dose comprises about 3×10¹⁴ vg/kg or less, about 2×10¹⁴ vg/kg or less, about 1×10¹⁴ vg/kg or less, about 9×10¹³ vg/kg or less, about 8×10¹³ vg/kg or less, about 7×10¹³ vg/kg or less, about 6×10¹³ vg/kg or less, about 5×10¹³ vg/kg or less, about 4×10¹³ vg/kg or less, about 3×10¹³ vg/kg or less, about 2×10¹³ vg/kg or less, or about 1×10¹³ vg/kg or less.

Embodiment 173: A method of alleviating one or more symptoms of a heart disease or disorder in a subject in need thereof comprising administering the rAAV virion of any one of embodiments 1 to 56 or the composition of embodiment 108.

Embodiment 174: A method of improving one or more symptoms of a heart disease or disorder in a subject in need thereof comprising administering the rAAV virion of any one of embodiments 1 to 56 or the composition of embodiment 108.

Embodiment 175: A method of preventing one or more symptoms of a heart disease or disorder in a subject in need thereof comprising administering the rAAV virion of any one of embodiments 1 to 56 or the composition of embodiment 108.

Embodiment 176: An expression cassette comprising a polynucleotide comprising a 5′ to 3′ arrangement of elements, wherein the elements comprise:
i. one or more promoters;
ii. optionally one or more enhancers;
iii. optionally one or more introns;
iv. one or more transgenes;
v. optionally one or more WPRE sequences; and
vi. optionally one or more polyadenylation sequences, p(A).

Embodiment 177: The expression cassette of embodiment 176, wherein the 5′ to 3′ arrangement of elements is selected from:
i. 5′-promoter-intron-transgene-WPRE-p(A)-3′;
ii. 5′-enhancer-promoter-transgene-WPRE-p(A)-3′;
iii. 5′-enhancer-enhancer-promoter-transgene-WPRE-p(A)-3′;
iv. 5′-enhancer-enhancer-promoter-intron-transgene-WPRE-p(A)-3′;
v. 5′-enhancer-enhancer-promoter-intron-transgene-WPRE-p(A)-3′;
vi. 5′-enhancer-promoter-intron-transgene-WPRE-p(A)-p(A)-transgene-intron-promoter-enhancer-3′;
vii. 5′-enhancer-promoter-intron-transgene-WPRE-p(A)-enhancer-promoter-intron-transgene-p(A)-3′;
viii. 5′-p(A)-WPRE-transgene-intron-promoter-enhancer-enhancer-promoter-intron-transgene-p(A)-3′;
ix. 5′-promoter-intron-transgene-WPRE-p(A)-p(A)-transgene-intron-promoter-3′;
x. 5′-promoter-intron-transgene-WPRE-p(A)-promoter-intron-transgene-p(A)-3′; and
xi. 5′-p(A)-WPRE-transgene-intron-promoter-promoter-intron-transgene-p(A)-3′.

Embodiment 178: The expression cassette of embodiment 176 or embodiment 177, wherein the transgene has an increased expression level compared to a second expression cassette comprising a polynucleotide having an arrangement of elements from 5′ to 3′ comprising: 5′-promoter-transgene-WPRE-p(A)-3′.

Embodiment 179: The expression cassette of embodiment 178, wherein the increased expression level is between about 1.5-fold and about 150-fold compared to the second expression cassette.

Embodiment 180: A recombinant adeno-associated virus (rAAV) virion, comprising a capsid protein and a viral genome comprising an expression cassette of any one of embodiments 176 to 179, the expression cassette flanked by inverted terminal repeats.

Embodiment 181: The rAAV of embodiment 180, wherein the expression cassette comprises a transgene, wherein the transgene encodes a polypeptide use for treating or a preventing a heart disease, or alleviating symptoms associated with a heart disease.

Embodiment 182: The rAAV of embodiment 180 or embodiment 181, wherein the capsid protein is selected from any one of SEQ ID NOs: 145-200.

### Numbered Embodiments of the Invention II

Embodiment 1: A recombinant adeno-associated virus (rAAV) virion, comprising a capsid protein and a viral genome comprising an expression cassette comprising a polynucleotide sequence encoding a polypeptide operatively linked to a promoter, the expression cassette flanked by inverted terminal repeats, optionally wherein the polypeptide is for expression in a cardiac cell or tissue and/or for use in treating or a preventing a heart disease.

Embodiment 2: The rAAV virion of embodiment 1, wherein the polypeptide is selected from DWORF, JPH2, BAG3, CRYAB, Lamin A isoform of LMNA, Lamin C isoform of LMNA, TNNI3, PLN, LAMP2a, LAMP2b, LAMP2c, DPI isoform of DSP, DPII isoform of DSP, DSG2, and JUP.

Embodiment 3: The rAAV virion of embodiment 1 or 2, wherein the polypeptide shares at least 90%, 95%, 96%, 97%, 98%, 99% or 100% sequence identity to a sequence selected from polypeptide sequences in Tables 2a and 2b.

Embodiment 4: The rAAV virion of any one of embodiments 1-3, wherein the promoter is a chicken cTnT promoter.

Embodiment 5: The rAAV virion of embodiment 4, wherein the chicken cTnT promoter shares at least 90%, 95%, 96%, 97%, 98%, or 99% identity to SEQ ID NO: 11.

Embodiment 6: The rAAV virion of embodiment 4, wherein the chicken cTnT promoter comprises SEQ ID NO: 11.

Embodiment 7: The rAAV virion of any one of embodiments 1-3, wherein the promoter is a human cTnT promoter.

Embodiment 8: The rAAV virion of embodiment 7, wherein the human cTnT promoter shares at least 90%, 95%, 96%, 97%, 98%, or 99% identity to SEQ ID NO: 12 or SEQ ID NO: 13.

Embodiment 9: The rAAV virion of embodiment 7, wherein the human cTnT promoter comprises SEQ ID NO: 12 or SEQ ID NO: 13.

Embodiment 10: The rAAV virion of any one of embodiments 1 to 9, wherein the expression cassette further comprises one or more enhancers.

Embodiment 11: The rAAV virion of embodiment 10, wherein the enhancer the one or more enhancers are selected from a ACTC1 cardiac enhancer and a αMHC enhancer.

Embodiment 12: The rAAV virion of embodiment 11, wherein the ACTC1 cardiac enhancer shares at least 90%, 95%, 96%, 97%, 98%, or 99% identity to SEQ ID NO: 78.

Embodiment 13: The rAAV virion of embodiment 11, wherein the ACTC1 cardiac enhancer comprises SEQ ID NO: 78.

Embodiment 14: The rAAV virion of embodiment 11, wherein the αMHC enhancer shares at least 90%, 95%, 96%, 97%, 98%, or 99% identity to SEQ ID NO: 79.

Embodiment 15: The rAAV virion of embodiment 11, wherein the αMHC enhancer comprises SEQ ID NO: 79.

Embodiment 16: The rAAV virion of any one of embodiments 1 to 15, wherein the expression cassette further comprises an intron.

Embodiment 17: The rAAV virion of embodiment 16, wherein the intron is selected from a CMV intron and a chimeric intron.

Embodiment 18: The rAAV virion of embodiment 17, wherein the CMV intron shares at least 90%, 95%, 96%, 97%, 98%, or 99% identity to SEQ ID NO: 80.

Embodiment 19: The rAAV virion of embodiment 17, wherein the CMV intron comprises SEQ ID NO: 80.

Embodiment 20: The rAAV virion of embodiment 17, wherein the chimeric intron shares at least 90%, 95%, 96%, 97%, 98%, or 99% identity to SEQ ID NO: 81.

Embodiment 21: The rAAV virion of embodiment 17, wherein the chimeric intron comprises SEQ ID NO: 81.

Embodiment 22: The rAAV virion of any one of embodiments 1 to 21, wherein the expression cassette further comprises a WPRE sequence.

Embodiment 23: The rAAV virion of embodiment 22, wherein the WPRE sequence shares at least 90%, 95%, 96%, 97%, 98%, or 99% identity to SEQ ID NO: 26.

Embodiment 24: The rAAV virion of embodiment 22, wherein the WPRE sequence comprises SEQ ID NO: 26.

Embodiment 25: The rAAV virion of any one of embodiments 1 to 24, wherein the expression cassette further comprises a polyadenylation sequence.

Embodiment 26: The rAAV virion of embodiment 25, wherein the polyadenylation sequence is selected from a BGH polyadenylation sequence and a SV40 polyadenylation sequence.

Embodiment 27: The rAAV virion of embodiment 26, wherein the BGH polyadenylation sequence shares at least 90%, 95%, 96%, 97%, 98%, or 99% identity to SEQ ID NO: 27.

Embodiment 28: The rAAV virion of embodiment 26, wherein the BGH polyadenylation sequence comprises SEQ ID NO: 27.

Embodiment 29: The rAAV virion of embodiment 26, wherein the SV40 polyadenylation sequence shares at least 90%, 95%, 96%, 97%, 98%, or 99% identity to SEQ ID NO: 28.

Embodiment 30: The rAAV virion of embodiment 26, wherein the SV40 polyadenylation sequence comprises SEQ ID NO: 28.

Embodiment 31: The rAAV virion of any one of embodiments 1 to 30, wherein the expression cassette is flanked by ITRs.

Embodiment 32: The rAAV virion of embodiment 31, wherein the ITRs share at least 90%, 95%, 96%, 97%, 98%, or 99% identity to one or more of SEQ ID NO: 14 and SEQ ID NO: 15.

Embodiment 33: The rAAV virion of embodiment 31, wherein the ITRs comprise one or more of SEQ ID NO: 14 and SEQ ID NO: 15.

Embodiment 34: The rAAV virion of any one of embodiments 1 to 33, wherein the expression cassette comprises a single promoter.

Embodiment 35: The rAAV virion of any one of embodiments 1 to 33, wherein the expression cassette comprises two promoters.

Embodiment 36: The rAAV virion of any one of embodiments 1 to 35, wherein the expression cassette comprises a single copy a sequence encoding the polypeptide.

Embodiment 37: The rAAV virion of any one of embodiments 1 to 35, wherein the expression cassette comprises two copies of a sequence encoding the polypeptide.

Embodiment 38: The rAAV virion of any one of embodiments 1 to 37, wherein the expression cassette comprises one, two, three, or four enhancers.

Embodiment 39: The rAAV virion of any one of embodiments 1 to 38, wherein the expression cassette comprises one or two introns.

Embodiment 40: The rAAV virion of any one of embodiments 1 to 39, wherein the expression cassette comprises one or two WPRE sequences.

Embodiment 41: The rAAV virion of any one of embodiments 1 to 40, wherein the expression cassette comprises one or two polyadenylation sequences.

Embodiment 42: The rAAV virion of any one of embodiments 1 to 41, wherein the expression cassette comprises about 3.2 kb, about, about 3.3 kb, about 3.4 kb, about 3.5 kb, about 3.6 kb, about 3.7 kb, or less.

Embodiment 43: The rAAV virion of any one of embodiments 1 to 42, wherein the expression cassette comprises about 1.9 kb, about 2.1 kb, about 2.2 kb, about 2.3 kb, about 2.4 kb, about 2.5 kb, about 2.6 kb, about 2.7 kb, about 2.8 kb, about 2.9 kb, about 3.0 kb, about 3.1 kb, about 3.2 kb, or more.

Embodiment 44: The rAAV virion of any one of embodiments 1-3, wherein the expression cassette comprises a polynucleotide sequence that shares at least 75%, 80%, 90%, 95%, 96%, 97%, 98%, 99%, or 100% sequence identity to any one of SEQ ID NOs: 20-24 or SEQ ID NOs: 45-75, optionally without the sequence or sequences encoding DWORF (open reading frame or open reading frames encoding DWORF). In some embodiments, where the expression cassette is for expression of a polypeptide other than DWORF, the polynucleotide sequence shares at least 75%, 80%, 90%, 95%, 96%, 97%, 98%, 99%, or 100% identity to a sequence within any one of SEQ ID NOs: 20-24 or SEQ ID NOs: 45-75 where the sequence or sequences encoding DWORF (open reading frame or open reading frames encoding DWORF) are replaced by a sequence or sequences encoding the polypeptide other than DWORF (e.g., any polypeptide described herein, such as any polypeptide listed in Table 2b which also provide sequences of such polypeptides).

Embodiment 45: The rAAV virion of any one of embodiments 1-3, wherein the expression cassette comprises any one of SEQ ID NOs: 20-24 or SEQ ID NOs: 45-75, optionally without the sequence or sequences encoding DWORF (open reading frame or open reading frames encoding DWORF). In some embodiments, where the expression cassette is for expression of a polypeptide other than DWORF, the polynucleotide sequence comprises a sequence within any one of SEQ ID NOs: 20-24 or SEQ ID NOs: 45-75 where the sequence or sequences encoding DWORF (open reading frame or open reading frames encoding DWORF) are replaced by a sequence or sequences encoding the polypeptide other than DWORF (e.g., any polypeptide described herein, such as any polypeptide listed in Table 2b which also provide sequences of such polypeptides).

Embodiment 46: The rAAV virion of any one of embodiments 1-3, wherein the expression cassette comprises a polynucleotide sequence that shares at least 75%, 80%, 90%, 95%, 96%, 97%, 98%, or 99% identity to any one of SEQ ID NO: 49, SEQ ID NO: 51, SEQ ID NO: 55, SEQ ID NO: 56, SEQ ID NO: 57, SEQ ID NO: 58, SEQ ID NO: 59 and SEQ ID NO: 60, optionally without the sequence or sequences encoding DWORF (open reading frame or open reading frames encoding DWORF). In some embodiments, where the expression cassette is for expression of a polypeptide other than DWORF, the polynucleotide sequence comprises a sequence sharing at least 75%, 80%, 90%, 95%, 96%, 97%, 98%, or 99% identity to SEQ ID NO: 49, SEQ ID NO: 51, SEQ ID NO: 55, SEQ ID NO: 56, SEQ ID NO: 57, SEQ ID NO: 58, SEQ ID NO: 59 and SEQ ID NO: 60 in which the sequence or sequences encoding DWORF (open reading frame or open reading frames encoding DWORF) are replaced by a sequence or sequences encoding the polypeptide other than DWORF (e.g., any polypeptide described herein, such as any polypeptide listed in Table 2b which also provide sequences of such polypeptides).

Embodiment 47: The rAAV virion of any one of embodiments 1-3, wherein the expression cassette comprises any one of SEQ ID NO: 49, SEQ ID NO: 51, SEQ ID NO: 55, SEQ ID NO: 56, SEQ ID NO: 57, SEQ ID NO: 58, SEQ ID NO: 59 and SEQ ID NO: 60, optionally without the sequence or sequences encoding DWORF (open reading frame or open reading frames encoding DWORF). In some embodiments, where the expression cassette is for expression of a polypeptide other than DWORF, the polynucleotide sequence comprises a sequence of SEQ ID NO: 49, SEQ ID NO: 51, SEQ ID NO: 55, SEQ ID NO: 56, SEQ ID NO: 57, SEQ ID NO: 58, SEQ ID NO: 59 and SEQ ID NO: 60 in which the sequence or sequences encoding DWORF (open reading frame or open reading frames encoding DWORF) are replaced by a sequence or sequences encoding the polypeptide other than DWORF (e.g., any polypeptide described herein, such as any polypeptide listed in Table 2b which also provide sequences of such polypeptides).

Embodiment 48: The rAAV virion of any one of embodiments 1-3, wherein the expression cassette comprises a polynucleotide sequence that shares at least 75%, 80%, 90%, 95%, 96%, 97%, 98%, or 99% identity to any one of SEQ ID NO: 55, SEQ ID NO: 56, SEQ ID NO: 57, and SEQ ID NO: 58, optionally without the sequence or sequences encoding DWORF (open reading frame or open reading frames encoding DWORF). In some embodiments, where the expression cassette is for expression of a polypeptide other than DWORF, the polynucleotide sequence comprises a sequence sharing at least 75%, 80%, 90%, 95%, 96%, 97%, 98%, or 99% identity to any one of SEQ ID NO: 55, SEQ ID NO: 56, SEQ ID NO: 57, and SEQ ID NO: 58 in which the sequence or sequences encoding DWORF (open reading frame or open reading frames encoding DWORF) are replaced by a sequence or sequences encoding the polypeptide other than DWORF (e.g., any polypeptide described herein, such as any polypeptide listed in Table 2b which also provide sequences of such polypeptides).

Embodiment 49: The rAAV virion of any one of embodiments 1-3, wherein the expression cassette comprises any one of SEQ ID NO: 55, SEQ ID NO: 56, SEQ ID NO: 57, and SEQ ID NO: 58, optionally without the sequence or sequences encoding DWORF (open reading frame or open reading frames encoding DWORF). In some embodiments, where the expression cassette is for expression of a polypeptide other than DWORF, the polynucleotide sequence comprises a sequence of any one of SEQ ID NO: 55, SEQ ID NO: 56, SEQ ID NO: 57, and SEQ ID NO: 58 in which the sequence or sequences encoding DWORF (open reading frame or open reading frames encoding DWORF) are replaced by a sequence or sequences encoding the polypeptide other than DWORF (e.g., any polypeptide described herein, such as any polypeptide listed in Table 2b which also provide sequences of such polypeptides).

Embodiment 50: The rAAV virion of any one of embodiments 1-3, wherein the expression cassette comprises a polynucleotide sequence that shares at least 75%, 80%, 90%, 95%, 96%, 97%, 98%, or 99% identity to any one of SEQ ID NO: 62, SEQ ID NO: 63, SEQ ID NO: 67, SEQ ID NO: 69, SEQ ID NO: 74, and SEQ ID NO: 75, optionally without the sequence or sequences encoding DWORF (open reading frame or open reading frames encoding DWORF). In some embodiments, where the expression cassette is for expression of a polypeptide other than DWORF, the polynucleotide sequence comprises a sequence sharing at least 75%, 80%, 90%, 95%, 96%, 97%, 98%, or 99% identity to any one of SEQ ID NO: 62, SEQ ID NO: 63, SEQ ID NO: 67, SEQ ID NO: 69, SEQ ID NO: 74, and SEQ ID NO: 75 in which the sequence or sequences encoding DWORF (open reading frame or open reading frames encoding DWORF) are replaced by a sequence or sequences encoding the polypeptide other than DWORF (e.g., any polypeptide described herein, such as any polypeptide listed in Table 2b which also provide sequences of such polypeptides).

Embodiment 51: The rAAV virion of any one of embodiments 1-3, wherein the expression cassette comprises any one of SEQ ID NO: 62, SEQ ID NO: 63, SEQ ID NO: 67, SEQ ID NO: 69, SEQ ID NO: 74, and SEQ ID NO: 75, optionally without the sequence or sequences encoding DWORF (open reading frame or open reading frames encoding DWORF). In some embodiments, where the expression cassette is for expression of a polypeptide other than DWORF, the polynucleotide sequence comprises a sequence of any one of SEQ ID NO: 62, SEQ ID NO: 63, SEQ ID NO: 67, SEQ ID NO: 69, SEQ ID NO: 74, and SEQ ID NO: 75 in which the sequence or sequences encoding DWORF (open reading frame or open reading frames encoding DWORF) are replaced by a sequence or sequences encoding the polypeptide other than DWORF (e.g., any polypeptide described herein, such as any polypeptide listed in Table 2b which also provide sequences of such polypeptides).

Embodiment 52: The rAAV virion of any one of embodiments 1 to 51, wherein the capsid protein shares at least 98%, at least 99%, or 100% identity to an AAV9 capsid protein (SEQ ID NO: 143).

Embodiment 53: The rAAV virion of any one of embodiments 1 to 51, wherein the capsid protein shares at least 98%, at least 99%, or 100% identity to an AAV5 capsid protein (SEQ ID NO: 144).

Embodiment 54: The rAAV virion of any one of embodiments 1 to 51, wherein the capsid protein is a chimeric capsid protein.

Embodiment 55: The rAAV virion of any one of embodiments 1 to 51, wherein the capsid protein is an AAV5/AAV9 chimeric capsid protein.

Embodiment 56: The rAAV virion of any one of embodiments 1 to 51, wherein the capsid protein is selected from any one of SEQ ID NOs: 145-200.

Embodiment 57: An expression cassette comprising a polynucleotide sequence encoding a polypeptide operatively linked to a promoter, optionally wherein the polypeptide is for expression in a cardiac cell or tissue and/or for use in treating or a preventing a heart disease.

Embodiment 58: The expression cassette of embodiment 57, wherein the polypeptide is selected from DWORF, JPH2, BAG3, CRYAB, Lamin A isoform of LMNA, Lamin C isoform of LMNA, TNNI3, PLN, LAMP2a, LAMP2b, LAMP2c, DPI isoform of DSP, DPII isoform of DSP, DSG2, and JUP, optionally wherein the polypeptide shares at least 90%, 95%, 96%, 97%, 98%, 99% or 100% sequence identity to a sequence selected from polypeptide sequences in Tables 2a and 2b.

Embodiment 59: The expression cassette of embodiment 57 or 58, wherein the promoter is a chicken cTnT promoter.

Embodiment 60: The expression cassette of embodiment 59, wherein the chicken cTnT promoter shares at least 90%, 95%, 96%, 97%, 98%, or 99% identity to SEQ ID NO: 11.

Embodiment 61: The expression cassette of embodiment 59, wherein the chicken cTnT promoter comprises SEQ ID NO: 11.

Embodiment 62: The expression cassette of embodiment 57 or 58, wherein the promoter is a human cTnT promoter.

Embodiment 63: The expression cassette of embodiment 62, wherein the human cTnT promoter shares at least 90%, 95%, 96%, 97%, 98%, or 99% identity to SEQ ID NO: 12 or SEQ ID NO: 13.

Embodiment 64: The expression cassette of embodiment 62, wherein the human cTnT promoter comprises SEQ ID NO: 12 or SEQ ID NO: 13.

Embodiment 65: The expression cassette of any one of embodiments 57 to 64, wherein the expression cassette further comprises one or more enhancers.

Embodiment 66: The expression cassette of embodiment 65, wherein the enhancer the one or more enhancers are selected from a ACTC1 cardiac enhancer and a αMHC enhancer.

Embodiment 67: The expression cassette of embodiment 66, wherein the ACTC1 cardiac enhancer shares at least 90%, 95%, 96%, 97%, 98%, or 99% identity to SEQ ID NO: 78.

Embodiment 68: The expression cassette of embodiment 66, wherein the ACTC1 cardiac enhancer comprises SEQ ID NO: 78.

Embodiment 69: The expression cassette of embodiment 66, wherein the αMHC enhancer shares at least 90%, 95%, 96%, 97%, 98%, or 99% identity to SEQ ID NO: 79.

Embodiment 70: The expression cassette of embodiment 66, wherein the αMHC enhancer comprises SEQ ID NO: 79.

Embodiment 71: The expression cassette of any one of embodiments 57 to 70, wherein the expression cassette further comprises an intron.

Embodiment 72: The expression cassette of any one of embodiments 57 to 70, wherein the intron is selected from a CMV intron and a chimeric intron.

Embodiment 73: The expression cassette of embodiment 72, wherein the CMV intron shares at least 90%, 95%, 96%, 97%, 98%, or 99% identity to SEQ ID NO: 80.

Embodiment 74: The expression cassette of embodiment 72, wherein the CMV intron comprises SEQ ID NO: 80.

Embodiment 75: The expression cassette of embodiment 72, wherein the chimeric intron shares at least 90%, 95%, 96%, 97%, 98%, or 99% identity to SEQ ID NO: 81.

Embodiment 76: The expression cassette of embodiment 72, wherein the chimeric intron comprises SEQ ID NO: 81.

Embodiment 77: The expression cassette of any one of embodiments 57 to 76, wherein the expression cassette further comprises a WPRE sequence.

Embodiment 78: The expression cassette of embodiment 77, wherein the WPRE sequence shares at least 90%, 95%, 96%, 97%, 98%, or 99% identity to SEQ ID NO: 26.

Embodiment 79: The expression cassette of embodiment 77, wherein the WPRE sequence comprises SEQ ID NO: 26.

Embodiment 80: The expression cassette of any one of embodiments 57 to 79, wherein the expression cassette further comprises a polyadenylation sequence.

Embodiment 81: The expression cassette of embodiment 80, wherein the polyadenylation sequence is selected from a BGH polyadenylation sequence and a SV40 polyadenylation sequence.

Embodiment 82: The expression cassette of embodiment 81, wherein the BGH polyadenylation sequence shares at least 90%, 95%, 96%, 97%, 98%, or 99% identity to SEQ ID NO: 27.

Embodiment 83: The expression cassette of embodiment 81, wherein the BGH polyadenylation sequence comprises SEQ ID NO: 27.

Embodiment 84: The expression cassette of embodiment 81, wherein the SV40 polyadenylation sequence shares at least 90%, 95%, 96%, 97%, 98%, or 99% identity to SEQ ID NO: 28.

Embodiment 85: The expression cassette of embodiment 81, wherein the SV40 polyadenylation sequence comprises SEQ ID NO: 28.

Embodiment 86: The expression cassette of any one of embodiments 57 to 85, wherein the expression cassette is flanked by ITRs.

Embodiment 87: The expression cassette of embodiment 86, wherein the ITRs share at least 90%, 95%, 96%, 97%, 98%, or 99% identity to one or more of SEQ ID NO: 14 and SEQ ID NO: 15.

Embodiment 88: The expression cassette of embodiment 86, wherein the ITRs comprise one or more of SEQ ID NO: 14 and SEQ ID NO: 15.

Embodiment 89: The expression cassette of any one of embodiments 57 to 88 comprising a single promoter.

Embodiment 90: The expression cassette of any one of embodiments 57 to 88 comprising two promoters.

Embodiment 91: The expression cassette of any one of embodiments 57 to 90 comprising a single copy a sequence encoding the polypeptide.

Embodiment 92: The expression cassette of any one of embodiments 57 to 90 comprising two copies of a sequence encoding the polypeptide.

Embodiment 93: The expression cassette of any one of embodiments 57 to 92 comprising one, two, three, or four enhancers.

Embodiment 94: The expression cassette of any one of embodiments 57 to 93 comprising one or two introns.

Embodiment 95: The expression cassette of any one of embodiments 57 to 94 comprising one or two WPRE sequences.

Embodiment 96: The expression cassette of any one of embodiments 57 to 95 comprising one or two polyadenylation sequences.

Embodiment 97: The expression cassette of any one of embodiments 57 to 96 comprising about 3.2 kb, about, about 3.3 kb, about 3.4 kb, about 3.5 kb, about 3.6 kb, about 3.7 kb, or less.

Embodiment 98: The expression cassette of any one of embodiments 57 to 97 comprising about 1.9 kb, about 2.1 kb, about 2.2 kb, about 2.3 kb, about 2.4 kb, about 2.5 kb, about 2.6 kb, about 2.7 kb, about 2.8 kb, about 2.9 kb, about 3.0 kb, about 3.1 kb, about 3.2 kb, or more.

Embodiment 99: The expression cassette of embodiment 57 or 58, comprising a polynucleotide sequence that shares at least 75%, 80%, 90%, 95%, 96%, 97%, 98%, 99%, or 100% identity to any one of SEQ ID NOs: 20-24 or SEQ ID NOs: 45-75, optionally without the sequence or sequences encoding DWORF (open reading frame or open reading frames encoding DWORF).

In some embodiments, where the expression cassette is for expression of a polypeptide other than DWORF, the polynucleotide sequence shares at least 75%, 80%, 90%, 95%, 96%, 97%, 98%, 99%, or 100% identity to a sequence of any one of SEQ ID NOs: 20-24 or SEQ ID NOs: 45-75 in which the sequence or sequences encoding DWORF (open reading frame or open reading frames encoding DWORF) are replaced by a sequence or sequences encoding the polypeptide other than DWORF (e.g., any polypeptide described herein, such as any polypeptide listed in Table 2b which also provide sequences of such polypeptides).

Embodiment 100: The expression cassette of embodiment 57 or 58, comprising a polynucleotide sequence of any one of SEQ ID NOs: 20-24 or SEQ ID NOs: 45-75, optionally without the sequence or sequences encoding DWORF (open reading frame or open reading frames encoding DWORF). In some embodiments, where the expression cassette is for expression of a polypeptide other than DWORF, the polynucleotide sequence comprises any one of SEQ ID NOs: 20-24 or SEQ ID NOs: 45-75 in which the sequence or sequences encoding DWORF (open reading frame or open reading frames encoding DWORF) are replaced by a sequence or sequences encoding the polypeptide other than DWORF (e.g., any polypeptide described herein, such as any polypeptide listed in Table 2b which also provide sequences of such polypeptides).

Embodiment 101: The expression cassette of embodiment 57 or 58, comprising a polynucleotide sequence that shares at least 75%, 80%, 90%, 95%, 96%, 97%, 98%, or 99% identity to any one of SEQ ID NO: 49, SEQ ID NO: 51, SEQ ID NO: 55, SEQ ID NO: 56, SEQ ID NO: 57, SEQ ID NO: 58, SEQ ID NO: 59, and SEQ ID NO: 60, optionally without the sequence or sequences encoding DWORF (open reading frame or open reading frames encoding DWORF). In some embodiments, where the expression cassette is for expression of a polypeptide other than DWORF, the polynucleotide sequence shares at least 75%, 80%, 90%, 95%, 96%, 97%, 98%, or 99% identity to any one of SEQ ID NO: 49, SEQ ID NO: 51, SEQ ID NO: 55, SEQ ID NO: 56, SEQ ID NO: 57, SEQ ID NO: 58, SEQ ID NO: 59, and SEQ ID NO: 60 in which the sequence or sequences encoding DWORF (open reading frame or open reading frames encoding DWORF) are replaced by a sequence or sequences encoding the polypeptide other than DWORF (e.g., any polypeptide described herein, such as any polypeptide listed in Table 2b which also provide sequences of such polypeptides).

Embodiment 102: The expression cassette of embodiment 57 or 58, comprising a polynucleotide sequence of any one of SEQ ID NO: 49, SEQ ID NO: 51, SEQ ID NO: 55, SEQ ID NO: 56, SEQ ID NO: 57, SEQ ID NO: 58, SEQ ID NO: 59, and SEQ ID NO: 60, optionally without the sequence or sequences encoding DWORF (open reading frame or open reading frames encoding DWORF). In some embodiments, where the expression cassette is for expression of a polypeptide other than DWORF, the polynucleotide sequence comprises a sequence of any one of SEQ ID NO: 49, SEQ ID NO: 51, SEQ ID NO: 55, SEQ ID NO: 56, SEQ ID NO: 57, SEQ ID NO: 58, SEQ ID NO: 59, and SEQ ID NO: 60 in which the sequence or sequences encoding DWORF (open reading frame or open reading frames encoding DWORF) are replaced by a sequence or sequences encoding the polypeptide other than DWORF (e.g., any polypeptide described herein, such as any polypeptide listed in Table 2b which also provide sequences of such polypeptides).

Embodiment 103: The expression cassette of embodiment 57 or 58, comprising a polynucleotide sequence that shares at least 75%, 80%, 90%, 95%, 96%, 97%, 98%, or 99% identity to any one of SEQ ID NO: 55, SEQ ID NO: 56, SEQ ID NO: 57, and SEQ ID NO: 58, optionally without the sequence or sequences encoding DWORF (open reading frame or open reading frames encoding DWORF). In some embodiments, where the expression cassette is for expression of a polypeptide other than DWORF, the polynucleotide sequence shares at least 75%, 80%, 90%, 95%, 96%, 97%, 98%, or 99% identity to any one of SEQ ID NO: 55, SEQ ID NO: 56, SEQ ID NO: 57, and SEQ ID NO: 58 in which the sequence or sequences encoding DWORF (open reading frame or open reading frames encoding DWORF) are replaced by a sequence or sequences encoding the polypeptide other than DWORF (e.g., any polypeptide described herein, such as any polypeptide listed in Table 2b which also provide sequences of such polypeptides).

Embodiment 104: The expression cassette of embodiment 57, comprising any one of SEQ ID NO: 55, SEQ ID NO: 56, SEQ ID NO: 57, and SEQ ID NO: 58, optionally without the sequence or sequences encoding DWORF (open reading frame or open reading frames encoding DWORF). In some embodiments, where the expression cassette is for expression of a polypeptide other than DWORF, the polynucleotide sequence comprises a sequence of any one of SEQ ID NO: 55, SEQ ID NO: 56, SEQ ID NO: 57, and SEQ ID NO: 58 in which the sequence or sequences encoding DWORF (open reading frame or open reading frames encoding DWORF) are replaced by a sequence or sequences encoding the polypeptide other than DWORF (e.g., any polypeptide described herein, such as any polypeptide listed in Table 2b which also provide sequences of such polypeptides).

Embodiment 105: The expression cassette of embodiment 57 or 58, comprising a polynucleotide sequence that shares at least 75%, 80%, 90%, 95%, 96%, 97%, 98%, or 99% identity to any one of SEQ ID NO: 62, SEQ ID NO: 63, SEQ ID NO: 67, SEQ ID NO: 69, SEQ ID NO: 74, and SEQ ID NO: 75, optionally without the sequence or sequences encoding DWORF (open reading frame or open reading frames encoding DWORF). In some embodiments, where the expression cassette is for expression of a polypeptide other than DWORF, the polynucleotide sequence comprises a sequence that shares at least 75%, 80%, 90%, 95%, 96%, 97%, 98%, or 99% identity to any one of SEQ ID NO: 62, SEQ ID NO: 63, SEQ ID NO: 67, SEQ ID NO: 69, SEQ ID NO: 74, and SEQ ID NO: 75 in which the sequence or sequences encoding DWORF (open reading frame or open reading frames encoding DWORF) are replaced by a sequence or sequences encoding the polypeptide other than DWORF (e.g., any polypeptide described herein, such as any polypeptide listed in Table 2b which also provide sequences of such polypeptides).

Embodiment 106: The expression cassette of embodiment 57 or 58, comprising any one of SEQ ID NO: 62, SEQ ID NO: 63, SEQ ID NO: 67, SEQ ID NO: 69, SEQ ID NO: 74, and SEQ ID NO: 75, optionally without the sequence or sequences encoding DWORF (open reading frame or open reading frames encoding DWORF). In some embodiments, where the expression cassette is for expression of a polypeptide other than DWORF, the polynucleotide sequence comprises a sequence of any one of SEQ ID NO: 62, SEQ ID NO: 63, SEQ ID NO: 67, SEQ ID NO: 69, SEQ ID NO: 74, and SEQ ID NO: 75 in which the sequence or sequences encoding DWORF (open reading frame or open reading frames encoding DWORF) are replaced by a sequence or sequences encoding the polypeptide other than DWORF (e.g., any polypeptide described herein, such as any polypeptide listed in Table 2b which also provide sequences of such polypeptides).

Embodiment 107: The expression cassette of any one of embodiments 99 to 106, wherein the expression cassette does not comprise a 5′ inverted terminal repeat and a 3′ inverted terminal repeat of the polynucleotide sequence.

Embodiment 108: A pharmaceutical composition comprising the rAAV virion of any one of embodiments 1 to 56 and a pharmaceutically acceptable carrier, or a pharmaceutical composition comprising a vector comprising the expression cassette of any of embodiments 57 to 107 and a pharmaceutically acceptable carrier.

Embodiment 109: A kit comprising the pharmaceutical composition of embodiment 108.

Embodiment 110: A method of increasing a polypeptide expression in a cell comprising contacting a cell with the rAAV virion of any one of embodiments 1 to 56, a vector comprising the expression cassette of any one of embodiments 57-107, or the composition of embodiment 108.

Embodiment 111: The method of embodiment 110, wherein the cell is a cardiac cell.

Embodiment 112: The method of embodiment 111, wherein the cardiac cell is a cardiomyocyte.

Embodiment 113: The method of any one of embodiments 110 to 112, wherein the polypeptide expression is increased between about 1.5-fold and 150-fold.

Embodiment 114: The method of any one of embodiments 110 to 113, wherein the contacting is in vitro.

Embodiment 115: The method of any one of embodiments 110 to 113, wherein the contacting is in vivo.

Embodiment 116: A method of increasing a polypeptide expression in a tissue comprising contacting the tissue with the rAAV virion of any one of embodiments 1 to 56, a vector comprising the expression cassette of any one of embodiments 57-107, or the composition of embodiment 108.

Embodiment 117: The method of embodiment 116, wherein the tissue is cardiac tissue.

Embodiment 118: The method of embodiment 116 or 117, wherein polypeptide expression is increased between about 1.5-fold and 150-fold.

Embodiment 119: The method of any one of embodiments 116 to 118, wherein the contacting is in vitro.

Embodiment 120: The method of embodiment 116 or 118, wherein the contacting is in vivo.

Embodiment 121: A method of increasing a polypeptide expression in an organ comprising contacting the organ with the rAAV virion of any one of embodiments 1 to 56, a vector comprising the expression cassette of any one of embodiments 57-107, or the composition of embodiment 108.

Embodiment 122: The method of embodiment 121, wherein the organ is a heart.

Embodiment 123: The method of embodiment 122, wherein the heart is diseased or is at risk of disease.

Embodiment 124: The method of embodiment 122 or embodiment 123, wherein the heart has reduced or borderline ejection fraction.

Embodiment 125: The method of embodiment 122 or embodiment 123, wherein the heart has a normal ejection fraction.

Embodiment 126: The method of any one of embodiments 122 to 125, wherein the heart comprises a genetic mutation associated with a heart disease.

Embodiment 127: The method of embodiment 126, wherein the genetic mutation is a PLN mutation.

Embodiment 128: The method of any one of embodiments 121 to 127, wherein the heart has low or undetectable polypeptide expression compared to a healthy heart.

Embodiment 129: The method of any one of embodiments 121 to 128, wherein the polypeptide expression is increased between about 1.5-fold and 150-fold.

Embodiment 130: The method of any one of embodiments 121 to 129, wherein the contacting is in vitro.

Embodiment 131: The method of any one of embodiments 121 to 129, wherein the contacting is in vivo.

Embodiment 132: A method of increasing a polypeptide expression in an subject comprising administering to the subject the rAAV virion of any one of embodiments 1 to 56, a vector comprising the expression cassette of any one of embodiments 57-107, or the composition of embodiment 108.

Embodiment 133: The method of embodiment 132, wherein the subject is an animal.

Embodiment 134: The method of embodiment 132, wherein the subject is a human.

Embodiment 135: The method of any one of embodiments 132 to 134, wherein the polypeptide expression is increased in the heart of the subject.

Embodiment 136: The method of any one of embodiments 132 to 135, wherein subject has a heart disease or is at risk of a heart disease.

Embodiment 137: The method of any one of embodiments 132 to 136, wherein subject has borderline or reduced ejection fraction.

Embodiment 138: The method of any one of embodiments 132 to 136, wherein the subject has normal ejection fraction.

Embodiment 139: The method of any one of embodiments 132 to 138, wherein the subject has a genetic mutation associated with a heart disease.

Embodiment 140: The method of embodiment 139, wherein the genetic mutation is a PLN mutation.

Embodiment 141: The method of any one of embodiments 132 to 140, wherein the subject has a low or undetectable level of the polypeptide expression compared to a healthy subject.

Embodiment 142: A method of treating a heart disease or disorder in a subject in need thereof comprising administering to the subject the rAAV virion of any one of embodiments 1 to 56, a vector comprising the expression cassette of any one of embodiments 57-107, or the composition of embodiment 108.

Embodiment 143: The method of embodiment 142, wherein the subject has a heart disease or disorder.

Embodiment 144: The method of embodiment 142, wherein the subject is a risk of developing a heart disease or disorder.

Embodiment 145: The method or any one of embodiments 142 to 144, wherein the heart disease or disorder is cardiomyopathy.

Embodiment 146: The method of any one of embodiments 142 to 144, wherein the heart disease or disorder is dilated cardiomyopathy.

Embodiment 147: The method of any one of embodiments 142 to 144, wherein the heart disease or disorder is myocardial infarction.

Embodiment 148: The method of any one of embodiments 142 to 144, wherein the heart disease or disorder is chronic myocardial infarction.

Embodiment 149: The method of any one of embodiments 142 to 144, wherein the heart disease or disorder is acute myocardial infarction.

Embodiment 150: The method of any one of embodiments 142 to 149, wherein the subject has an inherited risk allele for a heart disease or disorder.

Embodiment 151: The method of any one of embodiments 142 to 150, wherein the inherited risk allele comprises a mutation to the PLN gene.

Embodiment 152: The method of embodiment 151, wherein the mutation to the PLN gene is a PLN promoter mutation.

Embodiment 153: The method of embodiment 151, wherein the mutation to the PLN gene is a PLN^{L39stop} mutation.

Embodiment 154: The method of embodiment 151, wherein the mutation to the PLN gene is a RC9 mutation.

Embodiment 155: The method of embodiment 151, wherein the mutation to the PLN gene is a R9L mutation.

Embodiment 156: The method of embodiment 151, wherein the mutation to the PLN gene is a PLN gene duplication.

Embodiment 157: The method of embodiment 151, wherein the mutation to the PLN gene is a R14del mutation.

Embodiment 158: The method of any one of embodiments 142 to 157, wherein the heart disease or disorder is with reduced ejection fraction (HFrEF).

Embodiment 159: The method of any one of embodiments 142 to 157, wherein the heart disease of disorder is with preserved ejection fraction (HFpEF).

Embodiment 160: The method of any one of embodiments 142 to 159, wherein the method causes expression of the polypeptide in the heart of the subject.

Embodiment 161: The method of any one of embodiments 142 to 160, wherein the method causes expression of the polypeptide in cardiomyocytes.

Embodiment 162: The method of any one of embodiments 142 to 161, wherein the method causes no detectable expression of the polypeptide in the muscles of the subject except the heart.

Embodiment 163: The method of any one of embodiments 142 to 162, wherein the method causes no detectable expression of the polypeptide in the liver of the subject.

Embodiment 164: The method of any one of embodiments 142 to 163, wherein the method causes no detectable expression of the polypeptide in cardiac fibroblasts.

Embodiment 165: The method of any one of embodiments 142 to 164, wherein the method improves one or more measures of cardiac function, optionally fraction shortening and/or left ventricular internal dimension (LVID).

Embodiment 166: The method of any one of embodiments 142 to 165, wherein the improvement in cardiac function is observed at weeks 2 through week 24.

Embodiment 167: The method of any one of embodiments 142 to 166, wherein the method reduces cardiac remodeling.

Embodiment 168: The method of any one of embodiments 142 to 166, wherein the method counteracts a decrease in the polypeptide expression in subjects suffering from or at risk of a heart disease.

Embodiment 169: The method of any one of embodiments 142 to 168, wherein the administering is by systemic administration.

Embodiment 170: The method of embodiment 169, wherein the systemic administration is selected from intravenous or intracoronary injection.

Embodiment 171: The method of embodiment 169 or 170, wherein the rAAV is administered as a unit dose.

Embodiment 172: The method of embodiment 171, wherein the unit dose comprises about 3×10¹⁴ vg/kg or less, about 2×10¹⁴ vg/kg or less, about 1×10¹⁴ vg/kg or less, about 9×10¹³ vg/kg or less, about 8×10¹³ vg/kg or less, about 7×10¹³ vg/kg or less, about 6×10¹³ vg/kg or less, about 5×10¹³ vg/kg or less, about 4×10¹³ vg/kg or less, about 3×10¹³ vg/kg or less, about 2×10¹³ vg/kg or less, or about 1×10¹³ vg/kg or less.

Embodiment 173: A method of alleviating one or more symptoms of a heart disease or disorder in a subject in need thereof comprising administering the rAAV virion of any one of embodiments 1 to 56, a vector comprising the expression cassette of any one of embodiments 57-107, or the composition of embodiment 108.

Embodiment 174: A method of improving one or more symptoms of a heart disease or disorder in a subject in need thereof comprising administering the rAAV virion of any one of embodiments 1 to 56, a vector comprising the expression cassette of any one of embodiments 57-107, or the composition of embodiment 108.

Embodiment 175: A method of preventing one or more symptoms of a heart disease or disorder in a subject in need thereof comprising administering the rAAV virion of any one of embodiments 1 to 56, a vector comprising the expression cassette of any one of embodiments 57-107, or the composition of embodiment 108.

Embodiment 176: An expression cassette comprising a polynucleotide comprising:
i. one or more promoters;
ii. optionally one or more enhancers;
iii. optionally one or more introns;
iv. one or more transgenes, optionally wherein the one or more transgenes encode one or more polypeptides for use in treating or a preventing a heart disease;
v. optionally one or more WPRE sequences; and
vi. optionally one or more polyadenylation sequences, p(A).

Embodiment 177: The expression cassette of embodiment 176, wherein the 5′ to 3′ arrangement of elements is selected from:
(i) 5′-promoter-transgene-WPRE-p(A)-3′;
(ii) 5′-promoter-intron-transgene-WPRE-p(A)-3′;
(iii) 5′-promoter-transgene-WPRE-p(A)-promoter-transgene-WPRE-p(A);
(iv) 5′-enhancer-promoter-transgene-WPRE-p(A)-3′;
(v) 5′-enhancer-promoter-intron-transgene-WPRE-p(A)-3′;
(vi) 5′-enhancer-enhancer-promoter-transgene-WPRE-p(A)-3′;
(vii) 5′-enhancer-enhancer-promoter-intron-transgene-WPRE-p(A)-3′;
(viii) 5′-enhancer-promoter-intron-transgene-WPRE-p(A)-p(A)-transgene-intron-promoter-enhancer-3′;
(ix) 5′-enhancer-promoter-intron-transgene-WPRE-p(A)-enhancer-promoter-intron-transgene-p(A)-3′;
(x) 5′-p(A)-WPRE-transgene-intron-promoter-enhancer-enhancer-promoter-intron-transgene-p(A)-3′;
(xi) 5′-promoter-intron-transgene-WPRE-p(A)-p(A)-transgene-intron-promoter-3′;
(xii) 5′-promoter-intron-transgene-WPRE-p(A)-promoter-intron-transgene-p(A)-3′; and
(xiii) 5′-p(A)-WPRE-transgene-intron-promoter-promoter-intron-transgene-p(A)-3′; wherein optionally the transgene encodes a polypeptide for use in treating or a preventing a heart disease.

Embodiment 178: The expression cassette of embodiment 176 or embodiment 177, wherein the transgene has an increased expression level compared to a second expression cassette comprising a polynucleotide having an arrangement of elements from 5′ to 3′ comprising: 5′-promoter-transgene-WPRE-p(A)-3′.

Embodiment 179: The expression cassette of embodiment 178, wherein the increased expression level is between about 1.5-fold and about 150-fold compared to the second expression cassette.

Embodiment 180: A recombinant adeno-associated virus (rAAV) virion, comprising a capsid protein and a viral genome comprising an expression cassette of any one of embodiments 176 to 179, the expression cassette flanked by inverted terminal repeats.

Embodiment 181: The rAAV of embodiment 180, wherein the expression cassette comprises a transgene, wherein the transgene encodes a polypeptide for use in treating or a preventing a heart disease, or alleviating symptoms associated with a heart disease.

Embodiment 182: The rAAV of embodiment 180 or embodiment 181 wherein the capsid protein is selected from any one of SEQ ID NOs: 145-200.

### EXAMPLES

### Example 1: Novel Expression Cassettes

The purpose of this study was to evaluate several engineered expression cassettes for their ability to express a transgene in human induced pluripotent stem cell derived cardiomyocytes (hiPSC-CM) and a mouse model.

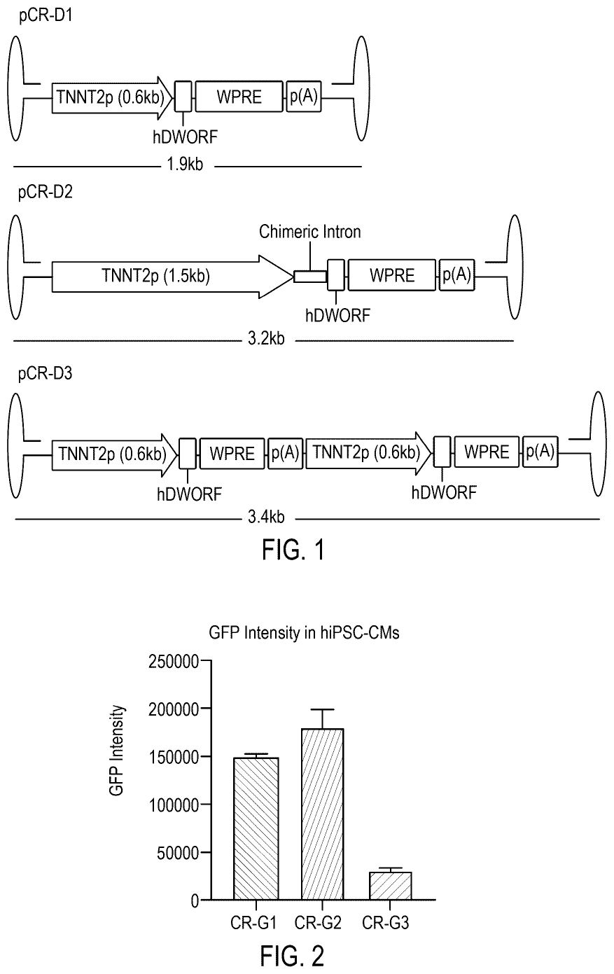

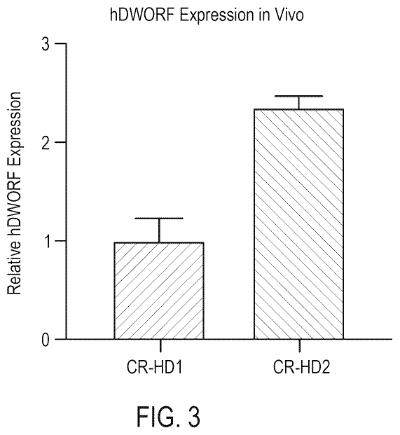

The human DWORF (hDWORF) polynucleotide (SEQ ID NO: 33) was inserted into expression cassettes designed to induce strong cardiomyocyte-specific expression while maintaining a total size of 3-4.7 kbp in length. Three unique expression cassettes were generated, pCR-HD1 (SEQ ID NO: 22), pCR-HD2 (SEQ ID NO: 23), and pCR-HD3. FIG. 1 shows an illustrative embodiment of each expression cassette, which contained iterations of a cardiac Troponin T promoter (see Table 3), WPRE (SEQ ID NO: 26) and BGH poly(A) signal (SEQ ID NO: 27). To evaluate expression of a transgene encoded by each expression cassette in hiPSC-CMs, the DWORF transgene was replaced with GFP in each expression cassettes, designated CR-G1, CR-G2, and CR-G3 corresponding to pCR-HD1, pCR-HD2, and pCR-HD3, respectively. The CR-G1, CR-G2, and CR-G3 expression cassettes were packaged into rAAV virions using an AAV6 capsid (SEQ ID NO: 39) protein. Cells were infected with each rAAV virion containing the expression cassette variants at an MOI of 50,000, and GFP expression was quantified by flow cytometry. FIG. 2 shows the pCR-G1, pCR-G2, and pCR-G3 expression cassettes showed high levels of expression in hiPSC-CMs. Surprisingly, the double transgene construct exhibited lower expression, likely due to the homologous recombination between the tandem repeat sequences. Next, the expression cassettes containing hDWORF, pCR-HD1 and pCR-HD2 were evaluated in a wild type CD1 mouse model. Each cassette was packaged into rAAV virions using an AAV9 capsid protein. The rAAV virions (5×10¹¹ vg) were administered systemically by retro-orbital injection into wild type CD1 mice (N=2). Two weeks following injection, animals were sacrificed and hDWORF expression was assessed in hearts. FIG. 3 shows the pCR-HD2 expression cassette results in about a 2-fold increase in hDWORF expression. It was concluded that increasing the length of the canonical hTNNT2 promoter from 600 bp to 1.5 kb, increasing total vector size from 1.9 kb to 3.2 kb, and/or inserting an intron surprisingly doubles gene expression.

### Example 2: Evaluation in hiPSC-CMs

The purpose of this study was to evaluate the ability of chimeric capsid proteins to facilitate infection of hiPSC-CMs with an rAAV virion containing an expression cassette encoding an HA-tagged hDWORF protein and the chimeric capsid protein.

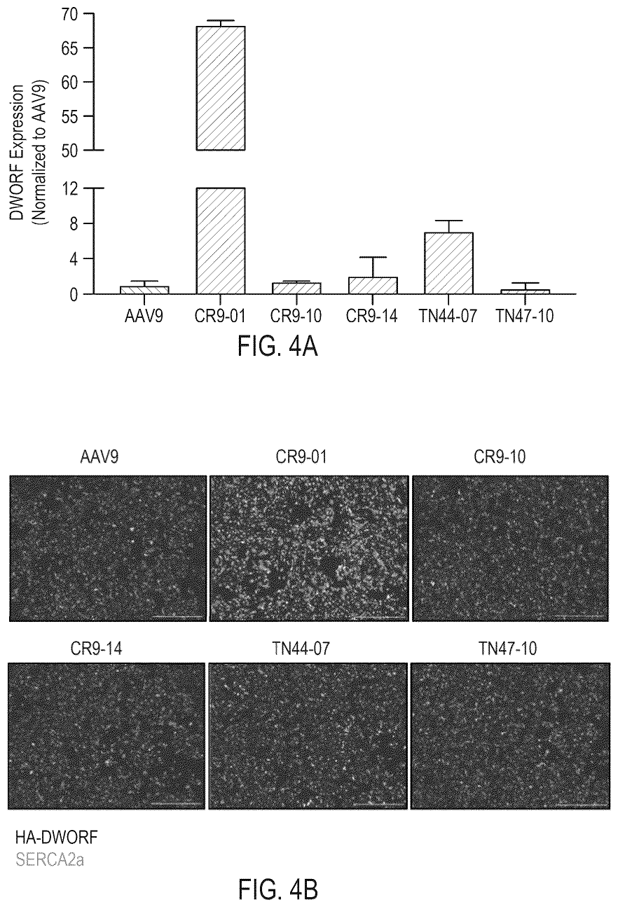

Expression cassettes encoding an HA-tagged hDWORF protein were packaged into rAAV virions with using a one of five chimeric capsid proteins. The chimeric capsid proteins tested include CR9-01 (SEQ ID NO: 29), CR9-10 (SEQ ID NO: 19), CR9-14 (SEQ ID NO: 30), TN44-07 (SEQ ID NO: 31), or TN47-10 (SEQ ID NO: 18), and the AAV9 capsid protein (SEQ ID NO: 16) was used as a control. Wild type hiPSC-CMs were infected at a MOI of 3,900. Five days following infection, cells were fixed and stained for SERCA2a and HA-tagged hDWORF. Stained cells were imaged (FIG. 4B) and quantified (FIG. 4A) using a cytation imager, and expression was normalized to AAV9. FIG. 4A shows HA-tagged hDWORF expression in hiPSC-CM cell cultures infected with rAAV virions containing the CR9-01, CR9-10, CR9-14, or TN44-07 chimeric capsid proteins. Each of these capsids performed as well or better than the AAV9 capsid protein in delivering a functional expression cassette.

### Example 3: Evaluation in a Murine Model

The purpose of this study was to evaluate the ability of chimeric capsid proteins to facilitate infection of heart tissue in wildtype mice with an rAAV virion containing an expression cassette encoding an HA-tagged hDWORF protein and the chimeric capsid protein.

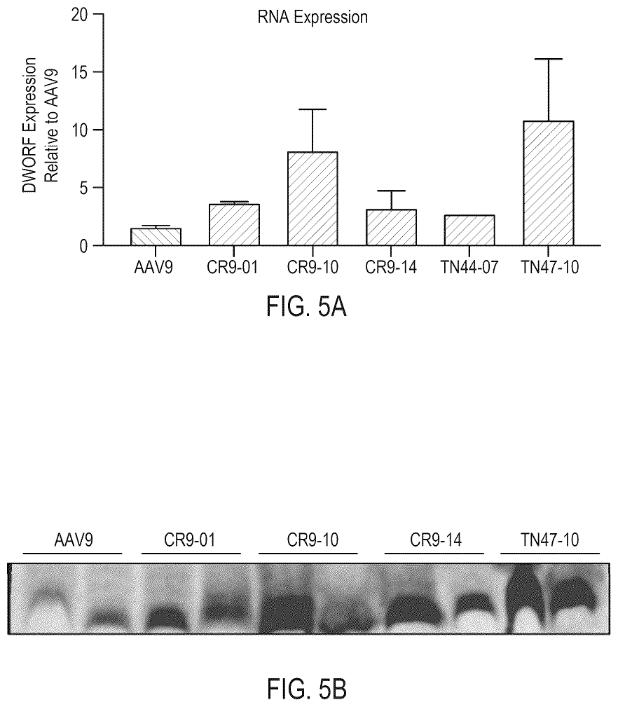

Expression cassettes encoding an HA-tagged hDWORF protein were packaged into rAAV virions with using one of five chimeric capsid proteins. The chimeric capsid proteins tested include CR9-01 (SEQ ID NO: 29), CR9-10 (SEQ ID NO: 19), CR9-14 (SEQ ID NO: 30), TN44-07 (SEQ ID NO: 31), or TN47-10 (SEQ ID NO: 18), and the AAV9 capsid protein (SEQ ID NO: 16) was used as a control. The rAAV virions were delivered by retro-orbital injection into wildtype mice at a dose of 5×10¹¹ vg/mouse (N=3). Fourteen days following injection, animals were sacrificed, and heart tissue was collected for RNA and protein analysis. FIG. 5A shows hDWORF RNA expression in heart tissue infected with the indicated capsid. All expression levels were normalized to the expression levels in the AAV9 group. The results indicate that in vivo delivery of rAAV virions packaged with the CR9-01, CR9-10, CR9-14, TN44-07, and TN47-10 each showed increased hDWORF expression compared to parental AAV9. The rAAV virions containing the CR9-10 and TN47-10 capsids showed the highest levels of expression compared to the other tested capsids (˜9-fold and -11-fold increased, respectively). FIG. 5B shows that hDWORF protein expression confirmed the RNA expression levels in the heart tissue, indicating that hDWORF RNA was translated to protein in the infected cells and that the modified capsids dramatically increased protein expression.

### Example 4: DWORF Gene Therapy in a PLN-R14^{Δ/Δ} A Model

The purpose of this study was to evaluate the ability of rAAV virions containing an expression cassette encoding an mDWORF protein to reduce symptoms associated with cardiomyopathy in mice harboring the PLN-R14 deletion mutation.

Transgenic mice expressing a homozygous PLN gene harboring the arginine 14 deletion (PLN-R14^{Δ/Δ}) recapitulates human cardiomyopathy, exhibiting similar histopathologic abnormalities and premature death (Haghighi K et al. Proc. Natl. Acad. Sci. U.S.A 103:1388-1393 (2006)). In particular, the transgenic mice can be used as a model of dilated cardiomyopathy. The model is significantly more severe than a MLP^{-/-} knockout and clinically relevant, as there are no known MLP knockout cardiomyopathy patients.

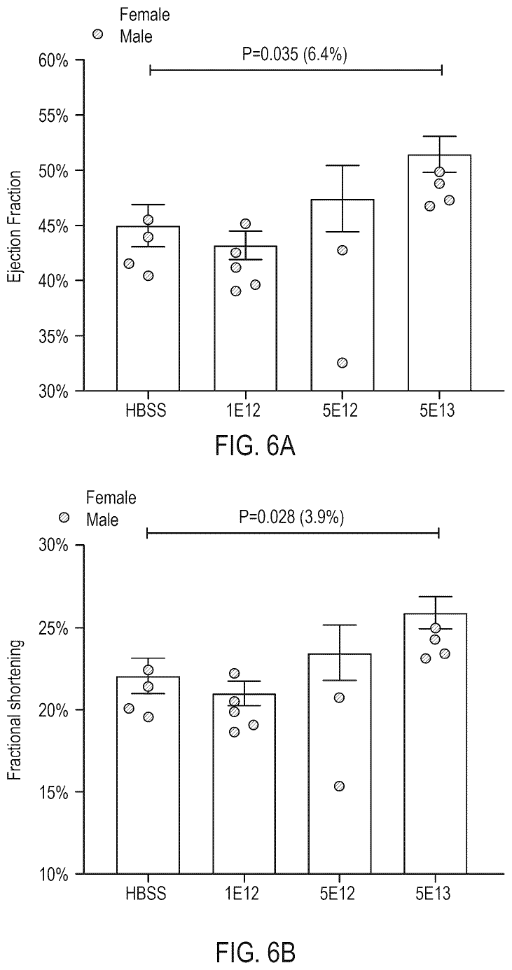

In this study, three-week-old homozygous PLN-R14del animals were injected with either Hank's Balanced Salt Solution (HBSS) as sham control or rAAV virions comprised of the pCR-MD1 expression cassette (SEQ ID NO: 20) and the AAV9 capsid protein. PLN-R14^{Δ/Δ} mice were administered rAAV virions by retro-orbital injection with a dose ranging from 5×10¹² vg/kg to 5×10¹³ vg/kg (N=7-8/group). Ejection fraction and fractional shortening were assessed by echocardiography as markers of cardiac function at 6.5 weeks of age. FIG. 6A show a dose-dependent improvement in ejection fraction compared to sham control (p=0.035). FIG. 6B shows a dose-dependent improvement in fractional shortening compared to sham control (p=0.028).

Together these results indicate systemic administration of rAAV virions expressing DWORF improves cardiac function in an animal model of cardiomyopathy. Observation of this effect in mice treated at three weeks of age demonstrates that this gene therapy can both treat and prevent development of hypertrophic cardiomyopathy (HCM).

### Example 5: rAAV-Medicated DWORF Expression

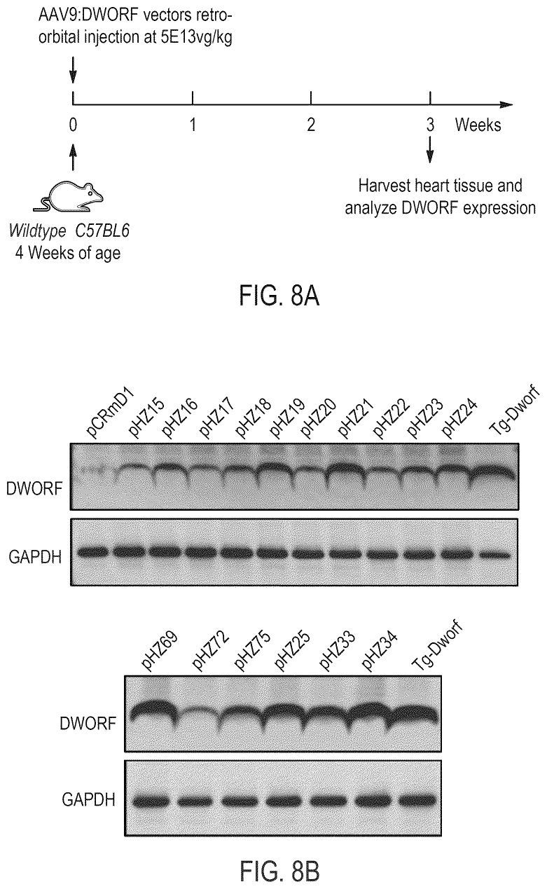

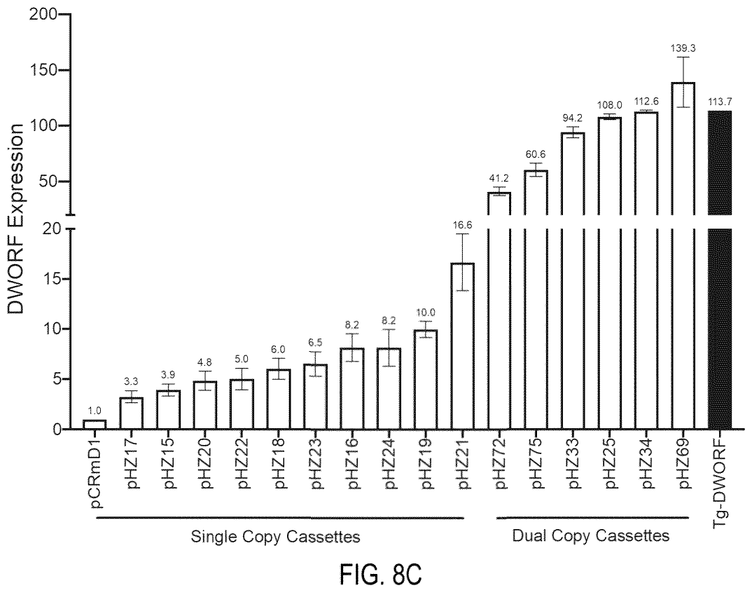

Expression cassettes were packaged with AAV9 capsid into rAAV virions and injected into 4 week old wildtype C57B16 mice at a dose of 5×10¹³ vg/kg (N=4). Mouse hearts were harvested at 3 weeks following injection. DWORF protein expression was determined by Western blots and quantified (FIG. 8C, Table 10). A transgenic mouse (Tg-dworf) with a high DWORF expression level was used as a positive control.

The results show that combinations of specific promoters and enhancers and their orientation in the expression cassette lead to different levels of DWORF expression. The specific expression cassettes used in this example and the associated elements and their orientation are shown in FIG. 7A-7C. DWORF expression was increased with added elements compared to promoter alone, but the extent of increased DWORF expression was not predictable based on elements and orientation alone.

FIG. 8A shows the strategy for evaluating the expression of transgene (DWORF) in cardiomyocytes of naïve mice. Three weeks post-injection, animals were sacrificed and DWORF expression was assessed in hearts. FIG. 8B shows expression analysis results for 16 AAV constructs with various regulatory elements and arrangements to increase CM-selective expression of transgene (DWORF). In particular, FIG. 8B shows the expression of DWORF in wild type C57BL6 mouse cardiomyocytes harvested three weeks after retro-orbital inject with AAV9:DWORF vectors as assessed by western blot; the lower western blot panels display the expression of GAPDH as a positive control. Quantification of these data in FIG. 8C revealed that combinations of specific promoters and enhancers and their orientation in the expression cassette result in different levels of transgene expression. A transgenic mouse (Tg-dworf) with a high DWORF expression level was used as a positive control in this example.

For each expression cassette, DWORF expression was normalized to the observed level for the pCRmD1 expression cassette, which has only the human cTnT promoter without added enhancer elements. Adding the ACTC1 enhancer (pHZ15) or αMHC enhancer (pHZ17) increased DWORF expression about 3-fold to about 4-fold relative to pCRmD1. Adding the CMV intron (pHZ20) was observed to increase DWORF expression about 5-fold. Combining the promoter, a single enhancer, and an intron (pHZ22 and pHZ23) did not significantly increase DWORF expression compared to any element alone. Combining both enhancers with the promoter only marginally increased DWORF expression to about 6- to 8-fold compared to promoter alone. Surprising, the combination of both enhancers, a promoter, and an intron increased DWORF expression about 10- to 16-fold. Interestingly, the 5′ to 3′ order of enhancers plays a fine-tune role in regulating protein expression. Orienting the ACTC1 enhancer 5′ to the second enhancer (pHZ16 or pHZ21) appears to increase DWORF expression compared to orienting the ACTC1 enhancer 3′ to the second enhancer (pHZ18 or pHZ19). Including a codon optimized DWORF transgene (pHZ24) also increased expression.

TABLE 10
DWORF Expression

| | DWORF |
|---|---|
| | Expression |
| | Fold |
| Expression | Increase over |
| Cassette | Promoter Only |
| pCRmD1 | 1.0 |
| pHZ15 | 3.9 |
| pHZ16 | 8.2 |
| pHZ17 | 3.3 |
| pHZ18 | 6.0 |
| pHZ19 | 10.0 |
| pHZ20 | 4.8 |
| pHZ21 | 16.6 |
| pHZ22 | 5.0 |
| pHZ23 | 6.5 |
| pHZ24 | 8.2 |
| pHZ25 | 108.0 |
| pHZ33 | 94.2 |
| pHZ34 | 112.6 |
| pHZ69 | 139.3 |
| pHZ72 | 41.2 |
| pHZ75 | 60.6 |
| Tg-DWORF | 113.7 |
| (Positive Control) | |

Although adding various regulatory elements greatly increased DWORF expression, adding more copies of the transgene (pHZ25, pHZ33, pHZ34, pHZ69, pHZ72 and pHZ75) has an unexpected synergistic impact on DWORF levels. We used different enhancers, promoters, introns, and codon-optimized DWORF in each copy to avoid the homologous recombination between the tandem repeat sequences. The dual copies vectors have about 40- to 140-fold more expression than DWORF under transcriptional control of the promoter alone. Surprisingly, the orientation of the two copies has an unexpected role in regulating gene expression. For example, a tail-to-tail orientation shows the best expression, followed by head-to-head and head-to-tail arrangements. The results also suggest that different arrangements of the two copies of the transgene can be used to fine-tune the transgene expression.

### Example 6: Effect of DWORF Expression on Ejection Fraction

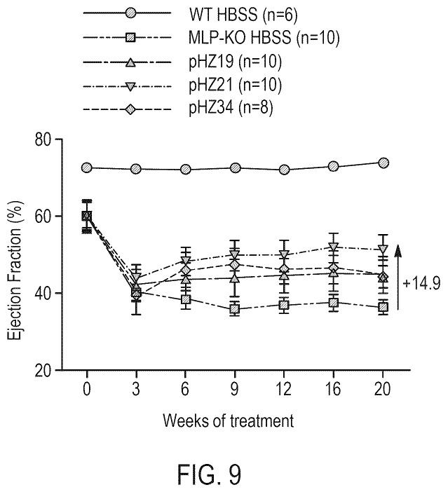

The effect of rAAV mediated DWORF expression on ejection fraction was determined in an MLP knockout (MLP-KO) dilated cardiomyopathy (DCM) model. Three of the rAAV virions were tested, including those with vector genomes having a single copy expression cassettes pHZ19 and pHZ21, and one having a dual copy expression cassette pHZ34. MLP-KO mice were dosed with either pHZ19 at 5×10¹³ vg/kg, pHZ21 at 5×10¹³ vg/kg, or pHZ34 at 1×10¹³ vg/kg. Virions were delivered by retro-orbital injection at 6 weeks of age, at which time the mice were presenting with moderate heart failure. Cardiac functions were accessed by echocardiography at 3, 6, 9, 12, 16 and 20 weeks post-treatment. As shown in FIG. 9, pHZ21 at a 5×10¹³ vg/kg dose significantly improved ejection fraction (14.4%) by 16 weeks post-virus injection compared to the HBSS group. Although pHZ21 only has a 1.6 fold more DWORF expression than the pHZ19, this modest improvement in expression seems to have a very profound effect on improving cardiac function. The dose of pHZ34 is five times lower than the pHZ19 and pHZ21, but it achieved similar improvement as the pHZ21 and better improvement than pHZ19, highlight the importance of DWORF expression.

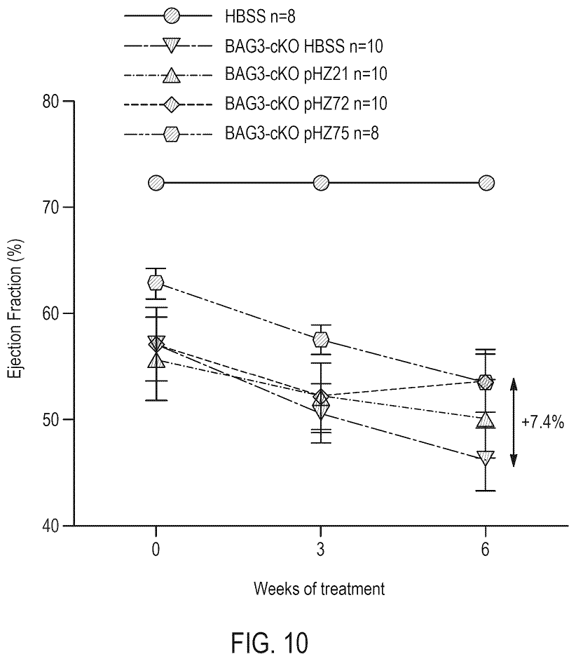

Three of the DWORF expression cassettes were tested in another well characterized DCM model, the BAG3 cardiac conditional knock-out (BAG3-cKO) model. DWORF expression cassettes were tested in this model included one single copy vector, the pHZ21, and two dual-copy vectors, pHZ72 and pHZ75. BAG3-cKO mice were dosed with either 5×10¹³ vg/kg AAV9-pHZ21, AAV9-pHZ72 and AAV9-pHZ75. Virions were delivered by retro-orbital injection at 8 weeks of age when the mice have already developed moderate heart failure. Cardiac functions were accessed by echocardiography at 3 and 6 weeks post-treatment. As shown in FIG. 10, pHZ72 at a 5×10¹³ vg/kg dose substantially improved ejection fraction (7.4%) by 6 weeks post-virus injection compared to the HBSS group, suggesting that such optimized DWORF gene therapy can improve cardiac function in this model.

### Example 7: Effect of DWORF Constructs in a Severe MLP-KO DCM Mouse Model

The purpose of this study was to test how optimized DWORF vectors can improve heart function and exercise capacity in a well-characterized MLP knockout (MLP-KO) dilated cardiomyopathy model.

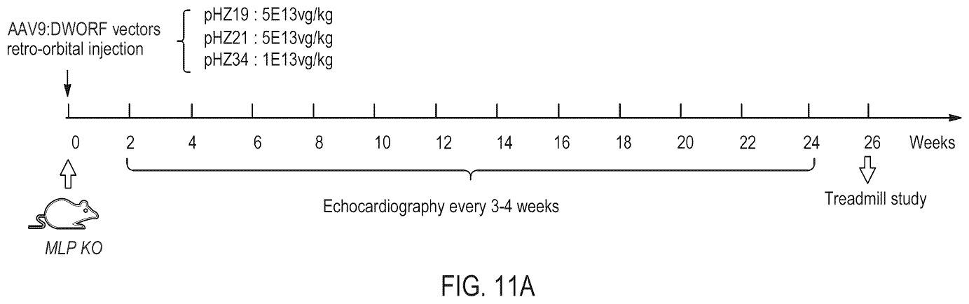

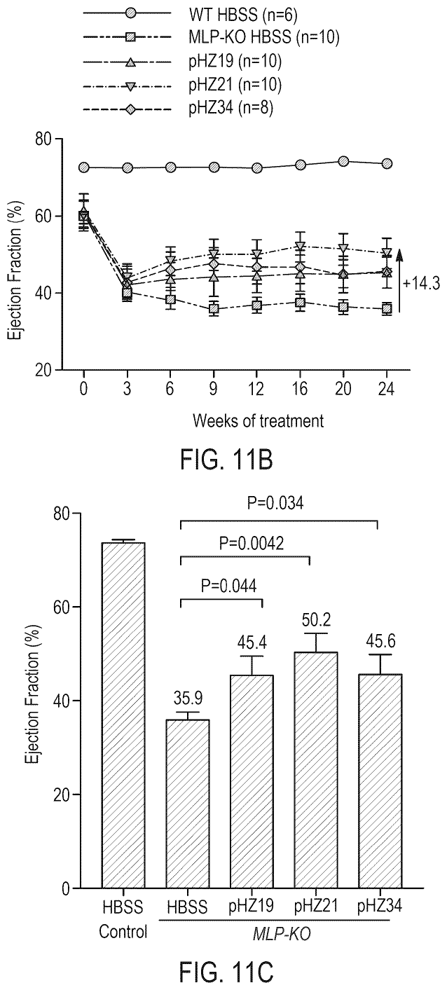

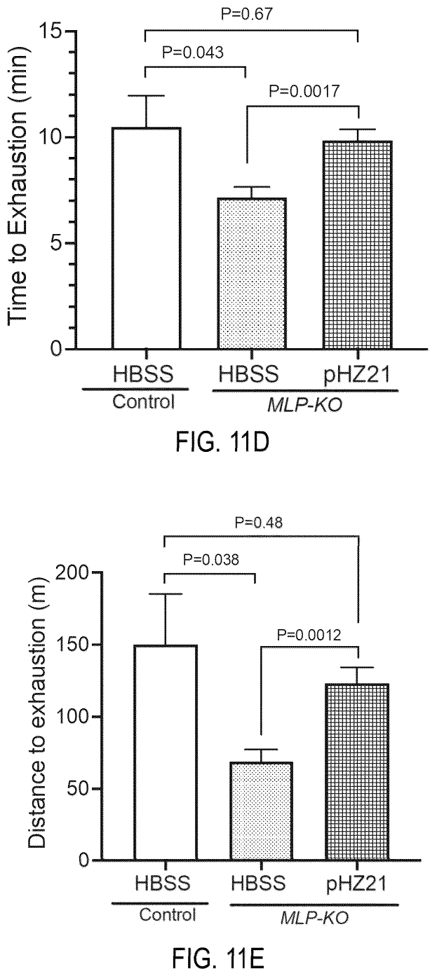

FIG. 11A is the schematic diagram of the study design. Three of the rAAV virions were tested, including those with vector genomes having a single copy expression cassettes pHZ19 and pHZ21, and one having a dual copy expression cassette pHZ34. MLP-KO mice were dosed with either pHZ19 at 5×10¹³ vg/kg, pHZ21 at 5×10¹³ vg/kg, or pHZ34 at 1×10¹³ vg/kg. Virions were delivered by retro-orbital injection at 6 weeks of age, at which time the mice were presenting with moderate heart failure. Cardiac functions were accessed by echocardiography 3-4 weeks and up to 24 weeks post-treatment. MLP-KO mice have limited exercise capacity compared to their wild-type littermates. To assess how much DWORF gene therapy can improve exercise capacity in MLP-KO mice, mice were allowed to run on a rodent treadmill and their running duration was monitored at 26 weeks.

As shown in FIG. 11B and FIG. 11C, pHZ21 at a 5×10¹³ vg/kg dose significantly improved ejection fraction (14.3%) by 24 weeks post-virus injection compared to the HBSS group. Although pHZ21 only has a 1.6 fold more DWORF expression than the pHZ19, this modest improvement in expression seems to have a very profound effect on improving cardiac function. The dose of pHZ34 is five times lower than the pHZ19 and pHZ21, but it achieved similar improvement as the pHZ21 and better improvement than pHZ19, highlighting the importance of DWORF expression. FIG. 11D and FIG. 11E show that MLP-KO mice have limited exercise capacity compared to their wild-type littermates. pHZ21, the best DWORF vector to improve ejection fraction, also significantly improved exercise capacity, including running distance and time to exhaustion, in the MLP KO DCM mouse model 26 weeks post-treatment relative to the saline control.

Overall, this study shows that AAV-delivered DWORF mitigated the contractile dysfunction and improved exercise capacity in this MLP-KO DCM model. AAV:DWORF cassettes expressed higher levels of DWORF, supporting the most significant degree of efficacy that was durable out to 24 weeks. These results show that DWORF gene therapy can be used for normalizing calcium homeostasis and limiting disease progression.

### Example 8: Tolerability of DWORF Gene Therapy in NaÎve Mice

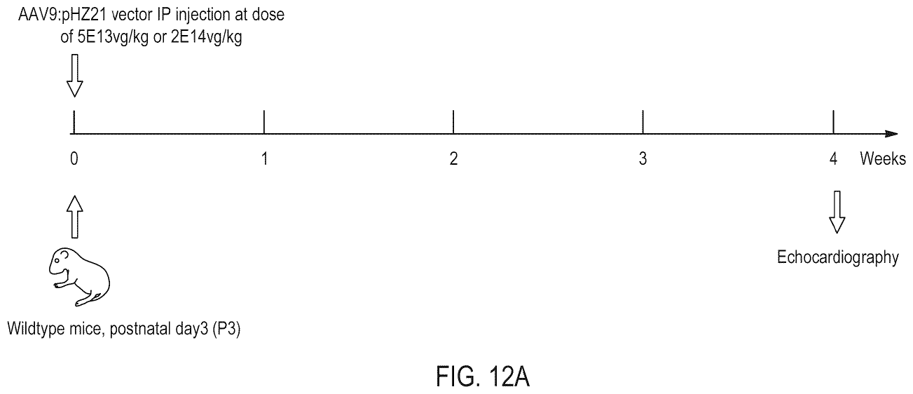

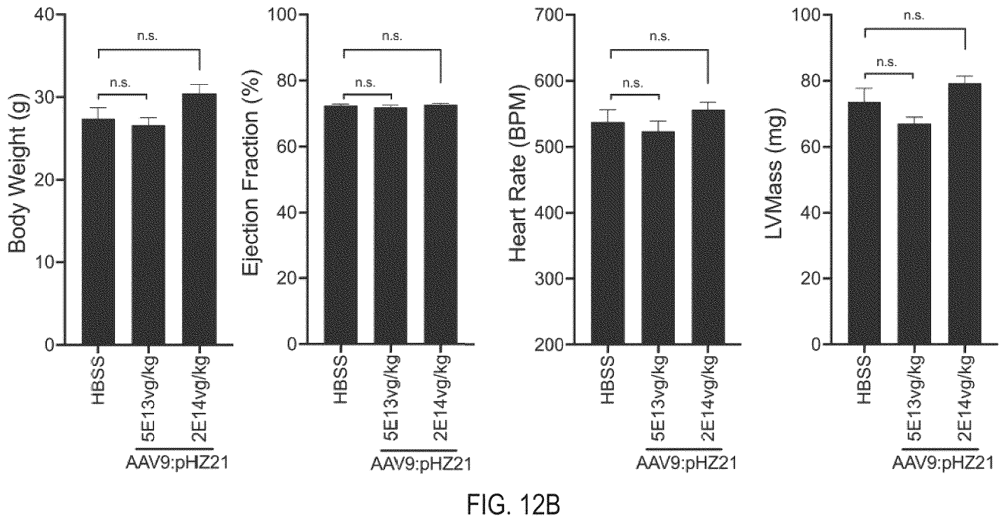

The purpose of this study was to evaluate the tolerability of DWORF gene therapy in naïve mice. FIG. 12A is the schematic diagram of the study design. Naïve mice were dosed with either HBSS saline control or AAV9:pHZ21 vector at two doses: 5×10¹³ vg/kg and 2×10¹⁴ vg/kg. Viruses were delivered via intraperitoneal (IP) injection at postnatal day 3 (P3). Cardiac functions were evaluated by echocardiography 4 weeks after viral injection.

FIG. 12B shows that AAV9:pHZ21 is well tolerated in wild-type mice up to 2×10¹⁴ vg/kg. Compared with the saline control group, there are no differences in body weight, ejection fraction, heart rate, and left ventricular mass (LV mass) for those groups that were dosed with AAV9:pHZ21.

### INCORPORATION BY REFERENCE

Various references such as patents, patent applications, and publications are cited herein, the disclosures of which are hereby incorporated herein by reference in their entireties. Also, all references mentioned herein are specifically incorporated by reference to disclose and describe the methods and/or materials in connection with which the publications are cited.

What is claimed is:

1. A method of treating a heart disease or disorder in a subject in need thereof, comprising administering to the subject a recombinant adeno-associated virus (rAAV) virion comprising (i) a capsid protein and (ii) a viral genome comprising an expression cassette, wherein the expression cassette comprises
a polynucleotide sequence encoding a dwarf open reading frame (DWORF) polypeptide operatively linked to a cardiac troponin T (cTnT) promoter, an actin, alpha cardiac muscle 1 (ACTC1) cardiac enhancer, and an alpha-myosin heavy chain (αMHC) enhancer,
wherein the expression cassette further comprises a polyadenylation sequence, and
wherein the ACTC1 cardiac enhancer comprises a polynucleotide sequence that is at least 95% identical to SEQ ID NO: 78.

2. The method of claim 1, wherein the DWORF polypeptide is a human DWORF polypeptide or a functional variant thereof sharing at least 80%, at least 90%, or 100% sequence identity to SEQ ID NO: 3.

3. The method of claim 1, wherein the cTnT promoter is a human cTnT promoter.

4. The method of claim 1, wherein the human cTnT promoter comprises a polynucleotide sequence that shares at least 90%, 95%, 96%, 97%, 98%, 99%, or 100% identity to SEQ ID NO: 12.

5. The method of claim 1, wherein the αMHC enhancer comprises a polynucleotide sequence that shares at least 90%, 95%, 96%, 97%, 98%, 99%, or 100% identity to SEQ ID NO: 79.

6. The method of claim 1, wherein the ACTC1 cardiac enhancer comprises the polynucleotide sequence of SEQ ID NO: 78.

7. The method of claim 1, wherein the expression cassette further comprises an intron.

8. The method of claim 7, wherein the intron is selected from a CMV intron and a chimeric intron.

9. The method of claim 8, wherein the CMV intron comprises a polynucleotide sequence that shares at least 90%, 95%, 96%, 97%, 98%, 99%, or 100% identity to SEQ ID NO: 80, and wherein the chimeric intron comprises a polynucleotide sequence that shares at least 90%, 95%, 96%, 97%, 98%, 99%, or 100% identity to SEQ ID NO: 81.

10. The method of claim 1, wherein the expression cassette further comprises a woodchuck hepatitis virus post-transcriptional element (WPRE) sequence, wherein the WPRE sequence comprises a polynucleotide sequence that shares at least 90%, 95%, 96%, 97%, 98%, 99%, or 100% identity to SEQ ID NO: 26.

11. The method of claim 1, wherein the polyadenylation sequence is selected from a BGH polyadenylation sequence and an SV40 polyadenylation sequence.

12. The method of claim 11, wherein the BGH polyadenylation sequence comprises a polynucleotide sequence that shares at least 90%, 95%, 96%, 97%, 98%, 99%, or 100% identity to SEQ ID NO: 27, and wherein the SV40 polyadenylation sequence comprises a polynucleotide sequence that shares at least 90%, 95%, 96%, 97%, 98%, 99%, or 100% identity to SEQ ID NO: 28.

13. The method of claim 1, comprising a single copy of the polynucleotide sequence encoding the DWORF polypeptide.

14. The method of claim 1, wherein the expression cassette comprises a single copy of the polynucleotide sequence encoding the DWORF polypeptide, wherein the DWORF polypeptide is a human DWORF polypeptide, wherein the cTnT promoter is a human cTnT promoter, and wherein the expression cassette further comprises a CMV intron and a BGH polyadenylation sequence.

15. The method of claim 14, wherein the expression cassette has a 5′ to 3′ arrangement of: the ACTC1 cardiac enhancer, the αMHC enhancer, the human cTnT promoter, the CMV intron, the polynucleotide sequence encoding the human DWORF polypeptide, and the BGH polyadenylation sequence; and wherein the ACTC1 cardiac enhancer, the αMHC enhancer, the human cTnT promoter, and the polynucleotide sequence encoding the human DWORF polypeptide are in the same orientation.

16. The method of claim 15, wherein the expression cassette further comprises a WPRE sequence between the polynucleotide sequence encoding the human DWORF polypeptide and the BGH polyadenylation sequence.

17. The method of claim 15, wherein the expression cassette comprises a polynucleotide sequence that shares at least 75%, 80%, 90%, 95%, 96%, 97%, 98%, 99%, or 100% sequence identity to SEQ ID NO: 69.

18. The method of claim 1, wherein the heart disease or disorder is cardiomyopathy, myocardial infarction, a heart disease or disorder with reduced ejection fraction (HFrEF), or a heart disease or disorder with preserved ejection fraction (HFpEF).

19. A method of increasing dwarf open reading frame (DWORF) expression in a cell, tissue, or organ, comprising contacting the cell, tissue, or organ with a recombinant adeno-associated virus (rAAV) virion comprising (i) a capsid protein and (ii) a viral genome comprising an expression cassette, wherein the expression cassette comprises
a polynucleotide sequence encoding a dwarf open reading frame (DWORF) polypeptide operatively linked to a cardiac troponin T (cTnT) promoter, an actin, alpha cardiac muscle 1 (ACTC1) cardiac enhancer, and an alpha-myosin heavy chain (αMHC) enhancer,
wherein the expression cassette further comprises a polyadenylation sequence, and wherein the ACTC1 cardiac enhancer comprises a polynucleotide sequence that is at least 95% identical to SEQ ID NO: 78.

20. The method of claim 19, wherein the DWORF polypeptide is a human DWORF polypeptide or a functional variant thereof sharing at least 80%, at least 90%, or 100% sequence identity to SEQ ID NO: 3.

21. The method of claim 19, wherein the human cTnT promoter comprises a polynucleotide sequence that shares at least 90%, 95%, 96%, 97%, 98%, 99%, or 100% identity to SEQ ID NO: 12.

22. The method of claim 19, wherein the αMHC enhancer comprises a polynucleotide sequence that shares at least 90%, 95%, 96%, 97%, 98%, 99%, or 100% identity to SEQ ID NO: 79.

23. The method of claim 18, wherein the ACTC1 cardiac enhancer comprises the polynucleotide sequence of SEQ ID NO: 78.

24. The method of claim 19, wherein the expression cassette further comprises an intron.

25. The method of claim 24, wherein the intron is selected from a CMV intron and a chimeric intron.

26. The method of claim 25, wherein the CMV intron comprises a polynucleotide sequence that shares at least 90%, 95%, 96%, 97%, 98%, 99%, or 100% identity to SEQ ID NO: 80, and wherein the chimeric intron comprises a polynucleotide sequence that shares at least 90%, 95%, 96%, 97%, 98%, 99%, or 100% identity to SEQ ID NO: 81.

27. The method of claim 19, wherein the expression cassette further comprises a woodchuck hepatitis virus post-transcriptional element (WPRE) sequence, wherein the WPRE sequence comprises a polynucleotide sequence that shares at least 90%, 95%, 96%, 97%, 98%, 99%, or 100% identity to SEQ ID NO: 26.

28. The method of claim 19, wherein the polyadenylation sequence is selected from a BGH polyadenylation sequence and an SV40 polyadenylation sequence.

29. The method of claim 28, wherein the BGH polyadenylation sequence comprises a polynucleotide sequence that shares at least 90%, 95%, 96%, 97%, 98%, 99%, or 100% identity to SEQ ID NO: 27, and wherein the SV40 polyadenylation sequence comprises a polynucleotide sequence that shares at least 90%, 95%, 96%, 97%, 98%, 99%, or 100% identity to SEQ ID NO: 28.

30. The method of claim 19, wherein the expression cassette comprises a single copy of the polynucleotide sequence encoding the DWORF polypeptide, wherein the DWORF polypeptide is a human DWORF polypeptide, wherein the cTnT promoter is a human cTnT promoter, and wherein the expression cassette further comprises a CMV intron and a BGH polyadenylation sequence.

31. The expression cassette of claim 30, wherein the expression cassette comprises a polynucleotide sequence that shares at least 75%, 80%, 90%, 95%, 96%, 97%, 98%, 99%, or 100% sequence identity to SEQ ID NO: 69.


SEQUENCE LISTING (230 sequences)

SEQ ID NO: 1 - protein, 34 aa, Mus musculus
MAEKESTSPHLMVPILLLVGWIVGCIIVIYIVFF

SEQ ID NO: 2 - DNA, 105 bp, Mus musculus
  source 1..105: mol_type = other DNA
atggctgagaaagagtcaacatcaccacacctcatggttcccattcttctcctggttggatggattgtaggctgcatcatcgttatttacattgtcttcttctaa

SEQ ID NO: 3 - protein, 35 aa, Homo sapiens
MAEKAGSTFSHLLVPILLLIGWIVGCIIMIYVVFS

SEQ ID NO: 4 - DNA, 108 bp, Homo sapiens
  source 1..108: mol_type = other DNA
atggctgaaaaagcggggtctacattttcacaccttctggttcctattcttctcctgattggctggattgtgggctgcatcataatgatttatgttgtcttctcttag

SEQ ID NO: 5 - protein, 35 aa, synthetic construct
  REGION 1..35: note = Recombinant DWORF variant
MAEKAESTSPHLMVPILLLVGWIVGCIIVIYIVFF

SEQ ID NO: 6 - DNA, 108 bp, synthetic construct
  misc_feature 1..108: note = Recombinant DWORF variant
  source 1..108: mol_type = other DNA
atggctgagaaagcagagtcaacatcaccacacctcatggttcccattcttctcctggttggatggattgtaggctgcatcatcgttatttacattgtcttcttctaa

SEQ ID NO: 7 - protein, 34 aa, synthetic construct
  REGION 1..34: note = Recombinant DWORF variant
MAEKESTSPHLIVPILLLVGWIVGCIIVIYIVFF

SEQ ID NO: 8 - DNA, 105 bp, synthetic construct
  misc_feature 1..105: note = Recombinant DWORF variant
  source 1..105: mol_type = other DNA
atggctgagaaagagtcaacatcaccacacctcattgttcccattcttctcctggttggatggattgtaggctgcatcatcgttatttacattgtcttcttctaa

SEQ ID NO: 9 - protein, 35 aa, synthetic construct
  REGION 1..35: note = Recombinant DWORF variant
MAEKAESTSPHLIVPILLLVGWIVGCIIVIYIVFF

SEQ ID NO: 10 - DNA, 108 bp, synthetic construct
  misc_feature 1..108: note = Recombinant DWORF variant
  source 1..108: mol_type = other DNA
atggctgagaaagcagagtcaacatcaccacacctcattgttcccattcttctcctggttggatggattgtaggctgcatcatcgttatttacattgtcttcttctaa

SEQ ID NO: 11 - DNA, 413 bp, synthetic construct
  misc_feature 1..413: note = Recombinant expression cassette or expression cassette component
  source 1..413: mol_type = other DNA
gggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtggga

SEQ ID NO: 12 - DNA, 600 bp, synthetic construct
  misc_feature 1..600: note = Recombinant expression cassette or expression cassette component
  source 1..600: mol_type = other DNA
gtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 13 - DNA, 1497 bp, synthetic construct
  misc_feature 1..1497: note = Recombinant expression cassette or expression cassette component
  source 1..1497: mol_type = other DNA
agaggaccctttcaaggacattagtggtggaggcagcatagtagctcccaaggcagagggattgagagaagagtttgaggactgggaaggcgggacacatgattgggtgatgggagaagggggcagagaatagcgagattgctttctttgcccacggagaaacagaggagtgtggatcatgaatgggcaagatctttaagtgccagggggggtcatggaggaggggagggcctgctccagaggaggaccattcctgcttcagagccaagcaggacctaggctgtgaagattcggagaaagagatggaggggagagtcagctcagctgcttactggcttgctttcctcctgtctctttcattttcataatctaccaaaccctgcaatgggccagccttgaacatacaagtgcatgtgcatggtcagacacaggcaagcaagcaagacccctaggcctgacctatgcatctgcaatctagtaggtttagcagatcatagccccgcactgcttgattttaaagccgttaggggatgacctttgacagtccgcatcacccctctcacacaacgagcgcctgttcaaggttcttgactggaagttctaccttgtatctggcctcctgtagcagtttcagtccattccctgtgaggagggtgtgccacatggctttgggggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcagctgctgttctgaggtaaggctcgggcagggctctggggaagaggagagcagagaatggacggggagatgtgagggtcttgggccctggcatatttacccagagtctgcctgtgtccgcagaagtccatggcccctcctggtggaggccacacttcagaggacaggttgccaggtctgggctccaagattggtacaatagagcagagaga

SEQ ID NO: 14 - DNA, 130 bp, synthetic construct
  misc_feature 1..130: note = Recombinant expression cassette or expression cassette component
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 15 - DNA, 130 bp, synthetic construct
  misc_feature 1..130: note = Recombinant expression cassette or expression cassette component
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 16 - protein, 735 aa, synthetic construct
  REGION 1..735: note = Recombinant expression cassette or expression cassette component
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRN

SEQ ID NO: 17 - DNA, 2209 bp, synthetic construct
  misc_feature 1..2209: note = Recombinant expression cassette or expression cassette component
  source 1..2209: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgt

SEQ ID NO: 18 - protein, 736 aa, synthetic construct
  REGION 1..736: note = Recombinant expression cassette or expression cassette component
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 19 - protein, 736 aa, synthetic construct
  REGION 1..736: note = Recombinant expression cassette or expression cassette component
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 20 - DNA, 1919 bp, synthetic construct
  misc_feature 1..1919: note = Recombinant expression cassette or expression cassette component
  source 1..1919: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaaggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagcggccgcccgccaccatggctgagaaagagtcaacatcaccacacctcatggttcccattcttctcctggttggatggattgtaggctgcatcatcgttatttacattgtcttcttctaaaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 21 - DNA, 3177 bp, synthetic construct
  misc_feature 1..3177: note = Recombinant expression cassette or expression cassette component
  source 1..3177: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagagaggaccctttcaaggacattagtggtggaggcagcatagtagctcccaaggcagagggattgagagaagagtttgaggactgggaaggcgggacacatgattgggtgatgggagaagggggcagagaatagcgagattgctttctttgcccacggagaaacagaggagtgtggatcatgaatgggcaagatctttaagtgccagggggggtcatggaggaggggagggcctgctccagaggaggaccattcctgcttcagagccaagcaggacctaggctgtgaagattcggagaaagagatggaggggagagtcagctcagctgcttactggcttgctttcctcctgtctctttcattttcataatctaccaaaccctgcaatgggccagccttgaacatacaagtgcatgtgcatggtcagacacaggcaagcaagcaagacccctaggcctgacctatgcatctgcaatctagtaggtttagcagatcatagccccgcactgcttgattttaaagccgttaggggatgacctttgacagtccgcatcacccctctcacacaacgagcgcctgttcaaggttcttgactggaagttctaccttgtatctggcctcctgtagcagtttcagtccattccctgtgaggagggtgtgccacatggctttgggggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcagctgctgttctgaggtaaggctcgggcagggctctggggaagaggagagcagagaatggacggggagatgtgagggtcttgggccctggcatatttacccagagtctgcctgtgtccgcagaagtccatggcccctcctggtggaggccacacttcagaggacaggttgccaggtctgggctccaagattggtacaatagagcagagagaggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcccaatcgatacccaatcgatacagatctagcggccgcgccgccaccatggctgagaaagagtcaacatcaccacacctcatggttcccattcttctcctggttggatggattgtaggctgcatcatcgttatttacattgtcttcttctaaccagaggttgattggatccaagctttggatccaatggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 22 - DNA, 1922 bp, synthetic construct
  misc_feature 1..1922: note = Recombinant expression cassette or expression cassette component
  source 1..1922: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaaggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagcggccgcccgccaccatggcagagaaggctggaagcactttctctcacctgctcgtgccgattttgcttttgattgggtggatagttggctgtatcataatgatctacgttgtcttttcatagaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 23 - DNA, 3180 bp, synthetic construct
  misc_feature 1..3180: note = Recombinant expression cassette or expression cassette component
  source 1..3180: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagagaggaccctttcaaggacattagtggtggaggcagcatagtagctcccaaggcagagggattgagagaagagtttgaggactgggaaggcgggacacatgattgggtgatgggagaagggggcagagaatagcgagattgctttctttgcccacggagaaacagaggagtgtggatcatgaatgggcaagatctttaagtgccagggggggtcatggaggaggggagggcctgctccagaggaggaccattcctgcttcagagccaagcaggacctaggctgtgaagattcggagaaagagatggaggggagagtcagctcagctgcttactggcttgctttcctcctgtctctttcattttcataatctaccaaaccctgcaatgggccagccttgaacatacaagtgcatgtgcatggtcagacacaggcaagcaagcaagacccctaggcctgacctatgcatctgcaatctagtaggtttagcagatcatagccccgcactgcttgattttaaagccgttaggggatgacctttgacagtccgcatcacccctctcacacaacgagcgcctgttcaaggttcttgactggaagttctaccttgtatctggcctcctgtagcagtttcagtccattccctgtgaggagggtgtgccacatggctttgggggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcagctgctgttctgaggtaaggctcgggcagggctctggggaagaggagagcagagaatggacggggagatgtgagggtcttgggccctggcatatttacccagagtctgcctgtgtccgcagaagtccatggcccctcctggtggaggccacacttcagaggacaggttgccaggtctgggctccaagattggtacaatagagcagagagaggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcccaatcgatacccaatcgatacagatctagcggccgcgccgccaccatggcagagaaggctggaagcactttctctcacctgctcgtgccgattttgcttttgattgggtggatagttggctgtatcataatgatctacgttgtcttttcatagccagaggttgattggatccaagctttggatccaatggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 24 - DNA, 1684 bp, synthetic construct
  misc_feature 1..1684: note = Recombinant expression cassette or expression cassette component
  source 1..1684: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagaattcgcccttacgggccccccctcgaggtcgggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacgatccccgaagctctagagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccgccaccatggctgagaaagagtcaacatcaccacacctcatggttcccattcttctcctggttggatggattgtaggctgcatcatcgttatttacattgtcttcttctaacggccgcgcggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 25 - DNA, 44 bp, synthetic construct
  misc_feature 1..44: note = Recombinant expression cassette or expression cassette component
  source 1..44: mol_type = other DNA
aaaaaaaaaaaaaaaaaaaaaaaaaaaaaaaaaaaaaaaaaaaa

SEQ ID NO: 26 - DNA, 540 bp, synthetic construct
  misc_feature 1..540: note = Recombinant expression cassette or expression cassette component
  source 1..540: mol_type = other DNA
tcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcg

SEQ ID NO: 27 - DNA, 215 bp, synthetic construct
  misc_feature 1..215: note = Recombinant expression cassette or expression cassette component
  source 1..215: mol_type = other DNA
gcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 28 - DNA, 134 bp, synthetic construct
  misc_feature 1..134: note = Recombinant expression cassette or expression cassette component
  source 1..134: mol_type = other DNA
gatccagacatgataagatacattgatgagtttggacaaaccacaactagaatgcagtgaaaaaaatgctttatttgtgaaatttgtgatgctattgctttatttgtaaccattataagctgcaataaacaagt

SEQ ID NO: 29 - protein, 736 aa, synthetic construct
  REGION 1..736: note = Recombinant expression cassette or expression cassette component
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTISQVNGRPRDLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 30 - protein, 736 aa, synthetic construct
  REGION 1..736: note = Recombinant expression cassette or expression cassette component
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 31 - protein, 727 aa, synthetic construct
  REGION 1..727: note = Recombinant expression cassette or expression cassette component
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPFGLVEEGAKTAPTGKRIDDHFPKRKKARTEEDSKPSTSSDAEAGPSGSQQLQIPAQPASSLGADTMSAGGGGPLGDNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 32 - protein, 35 aa, Homo sapiens; identical to SEQ ID NO: 3

SEQ ID NO: 33 - DNA, 108 bp, Homo sapiens
  source 1..108: mol_type = other DNA
atggcagagaaggctggaagcactttctctcacctgctcgtgccgattttgcttttgattgggtggatagttggctgtatcataatgatctacgttgtcttttcatag

SEQ ID NO: 34 - DNA, 2208 bp, synthetic construct
  misc_feature 1..2208: note = Recombinant expression cassette or expression cassette component
  source 1..2208: mol_type = other DNA
atggccgccgacggctacctgcccgactggctggaggacaacctgagcgagggcatcagggagtggtgggccctgaagcccggcgccccccagcccaaggccaaccagcagcaccaggacaacgccaggggcctggtgctgcccggctacaactacctgggccccggcaacggcctggacaggggcgagcccgtgaacagggccgacgaggtggccagggagcacgacatcagctacaacgagcagctggaggccggcgacaacccctacctgaagtacaaccacgccgacgccgagttccaggagaggctgaaggaggacaccagcttcggcggcaacctgggcagggccgtgttccaggccaagaagaggctgctggagcccctgggcctggtggaggaggccgccaagaccgcccccggcaagaagaggcccgtggagcagagcccccaggagcccgacagcagcgccggcatcggcaagagcggcgcccagcccgccaagaagaggctgaacttcggccagaccggcgacaccgagagcgtgcccgacccccagcccatcggcgagccccccgccgcccccagcggcgtgggcagcctgaccatggccagcggcggcggcgcccccgtggccgacaacaacgagggcgccgacggcgtgggcagcagcagcggcaactggcactgcgacagccagtggctgggcgacagggtgatcaccaccagcaccaggacctgggccctgcccacctacaacaaccacctgtacaagcagatcagcaacagcaccagcggcggcagcagcaacgacaacgcctacttcggctacagcaccccctggggctacttcgacttcaacaggttccactgccacttcagccccagggactggcagaggctgatcaacaacaactggggcttcaggcccaagaggctgaacttcaagctgttcaacatccaggtgaaggaggtgaccgacaacaacggcgtgaagaccatcgccaacaacctgaccagcaccgtgcaggtgttcaccgacagcgactaccagctgccctacgtgctgggcagcgcccacgagggctgcctgccccccttccccgccgacgtgttcatgatcccccagtacggctacctgaccctgaacgacggcagccaggccgtgggcaggagcagcttctactgcctggagtacttccccagccagatgctgaggaccggcaacaacttccagttcagctacgagttcgagaacgtgcccttccacagcagctacgcccacagccagagcctggacaggctgatgaaccccctgatcgaccagtacctgtactacctgagcaagaccatcaacggcagcggccagaaccagcagaccctgaagttcagcgtggccggccccagcaacatggccgtgcagggcaggaactacatccccggccccagctacaggcagcagagggtgagcaccaccgtgacccagaacaacaacagcgagttcgcctggcccggcgccagcagctgggccctgaacggcaggaacagcctgatgaaccccggccccgccatggccagccacaaggagggcgaggacaggttcttccccctgagcggcagcctgatcttcggcaagcagggcaccggcagggacaacgtggacgccgacaaggtgatgatcaccaacgaggaggagatcaagaccaccaaccccgtggccaccgagagctacggccaggtggccaccaaccaccaggccaactacggccaggcccagaccggctgggtgcagaaccagggcatcctgcccggcatggtgtggcaggacagggacgtgtacctgcagggccccatctgggccaagatcccccacaccgacggcaacttccaccccagccccctgatgggcggcttcggcatgaagcacccccccccccagatcctgatcaagaacacccccgtgcccgccgacccccccaccgccttcaacaaggacaagctgaacagcttcatcacccagtacagcaccggccaggtgagcgtggagatcgagtgggagctgcagaaggagaacagcaagaggtggaaccccgagatccagtacaccagcaactactacaagagcaacaacgtggagttcgccgtgaacaccgagggcgtgtacagcgagcccaggcccatcggcaccaggtacctgaccaggaacctg

SEQ ID NO: 35 - DNA, 2208 bp, synthetic construct
  misc_feature 1..2208: note = Recombinant expression cassette or expression cassette component
  source 1..2208: mol_type = other DNA
atggccgccgacggctacctgcccgactggctggaggacaacctgagcgagggcatcagggagtggtgggccctgaagcccggcgccccccagcccaaggccaaccagcagcaccaggacaacgccaggggcctggtgctgcccggctacaagtacctgggccccggcaacggcctggacaagggcgagcccgtgaacgccgccgacgccgccgccctggagcacgacaaggcctacgaccagcagctgaaggccggcgacaacccctacctgaagtacaaccacgccgacgccgagttccaggagaggctgaaggaggacaccagcttcggcggcaacctgggcagggccgtgttccaggccaagaagaggctgctggagcccctgggcctggtggaggaggccgccaagaccgcccccggcaagaagaggcccgtggagcagagcccccaggagcccgacagcagcgccggcatcggcaagagcggcgcccagcccgccaagaagaggctgaacttcggccagaccggcgacaccgagagcgtgcccgacccccagcccatcggcgagccccccgccgcccccagcggcgtgggcagcctgaccatggccagcggcggcggcgcccccgtggccgacaacaacgagggcgccgacggcgtgggcagcagcagcggcaactggcactgcgacagccagtggctgggcgacagggtgatcaccaccagcaccaggacctgggccctgcccacctacaacaaccacctgtacaagcagatcagcaacagcaccagcggcggcagcagcaacgacaacgcctacttcggctacagcaccccctggggctacttcgacttcaacaggttccactgccacttcagccccagggactggcagaggctgatcaacaacaactggggcttcaggcccaagaggctgaacttcaagctgttcaacatccaggtgaaggaggtgaccgacaacaacggcgtgaagaccatcgccaacaacctgaccagcaccgtgcaggtgttcaccgacagcgactaccagctgccctacgtgctgggcagcgcccacgagggctgcctgccccccttccccgccgacgtgttcatgatcccccagtacggctacctgaccctgaacgacggcagccaggccgtgggcaggagcagcttctactgcctggagtacttccccagccagatgctgaggaccggcaacaacttccagttcagctacgagttcgagaacgtgcccttccacagcagctacgcccacagccagagcctggacaggctgatgaaccccctgatcgaccagtacctgtactacctgagcaagaccatcaacggcagcggccagaaccagcagaccctgaagttcagcgtggccggccccagcaacatggccgtgcagggcaggaactacatccccggccccagctacaggcagcagagggtgagcaccaccgtgacccagaacaacaacagcgagttcgcctggcccggcgccagcagctgggccctgaacggcaggaacagcctgatgaaccccggccccgccatggccagccacaaggagggcgaggacaggttcttccccctgagcggcagcctgatcttcggcaagcagggcaccggcagggacaacgtggacgccgacaaggtgatgatcaccaacgaggaggagatcaagaccaccaaccccgtggccaccgagagctacggccaggtggccaccaaccaccaggccaactacggccaggcccagaccggctgggtgcagaaccagggcatcctgcccggcatggtgtggcaggacagggacgtgtacctgcagggccccatctgggccaagatcccccacaccgacggcaacttccaccccagccccctgatgggcggcttcggcatgaagcacccccccccccagatcctgatcaagaacacccccgtgcccgccgacccccccaccgccttcaacaaggacaagctgaacagcttcatcacccagtacagcaccggccaggtgagcgtggagatcgagtgggagctgcagaaggagaacagcaagaggtggaaccccgagatccagtacaccagcaactactacaagagcaacaacgtggagttcgccgtgaacaccgagggcgtgtacagcgagcccaggcccatcggcaccaggtacctgaccaggaacctg

SEQ ID NO: 36 - DNA, 2208 bp, synthetic construct
  misc_feature 1..2208: note = Recombinant expression cassette or expression cassette component
  source 1..2208: mol_type = other DNA
atggccgccgacggctacctgcccgactggctggaggacaacctgagcgagggcatcagggagtggtgggccctgaagcccggcgccccccagcccaaggccaaccagcagcaccaggacaacgccaggggcctggtgctgcccggctacaagtacctgggccccggcaacggcctggacaagggcgagcccgtgaacgccgccgacgccgccgccctggagcacgacaaggcctacgaccagcagctgaaggccggcgacaacccctacctgaagtacaaccacgccgacgccgagttccaggagaggctgaaggaggacaccagcttcggcggcaacctgggcagggccgtgttccaggccaagaagaggctgctggagcccctgggcctggtggaggaggccgccaagaccgcccccggcaagaagaggcccgtggagcagagcccccaggagcccgacagcagcgccggcatcggcaagagcggcgcccagcccgccaagaagaggctgaacttcggccagaccggcgacaccgagagcgtgcccgacccccagcccatcggcgagccccccgccgcccccagcggcgtgggcagcctgaccatggccagcggcggcggcgcccccgtggccgacaacaacgagggcgccgacggcgtgggcagcagcagcggcaactggcactgcgacagccagtggctgggcgacagggtgatcaccaccagcaccaggacctgggccctgcccacctacaacaaccacctgtacaagcagatcagcaacagcaccagcggcggcagcagcaacgacaacgcctacttcggctacagcaccccctggggctacttcgacttcaacaggttccactgccacttcagccccagggactggcagaggctgatcaacaacaactggggcttcaggcccaagaggctgaacttcaagctgttcaacatccaggtgaaggaggtgaccgacaacaacggcgtgaagaccatcgccaacaacctgaccagcaccgtgcaggtgttcaccgacagcgactaccagctgccctacgtgctgggcagcgcccacgagggctgcctgccccccttccccgccgacgtgttcatgatcccccagtacggctacctgaccctgaacgacggcagccaggccgtgggcaggagcagcttctactgcctggagtacttccccagccagatgctgaggaccggcaacaacttccagttcagctacgagttcgagaacgtgcccttccacagcagctacgcccacagccagagcctggacaggctgatgaaccccctgatcgaccagtacctgtactacctgagcaagaccatcagccaggtgaacggcaggcccagggacctgaagttcagcgtggccggccccagcaacatggccgtgcagggcaggaactacatccccggccccagctacaggcagcagagggtgagcaccaccgtgacccagaacaacaacagcgagttcgcctggcccggcgccagcagctgggccctgaacggcaggaacagcctgatgaaccccggccccgccatggccagccacaaggagggcgaggacaggttcttccccctgagcggcagcctgatcttcggcaagcagggcaccggcagggacaacgtggacgccgacaaggtgatgatcaccaacgaggaggagatcaagaccaccaaccccgtggccaccgagagctacggccaggtggccaccaaccaccagagcgcccaggcccaggcccagaccggctgggtgcagaaccagggcatcctgcccggcatggtgtggcaggacagggacgtgtacctgcagggccccatctgggccaagatcccccacaccgacggcaacttccaccccagccccctgatgggcggcttcggcatgaagcacccccccccccagatcctgatcaagaacacccccgtgcccgccgacccccccaccgccttcaacaaggacaagctgaacagcttcatcacccagtacagcaccggccaggtgagcgtggagatcgagtgggagctgcagaaggagaacagcaagaggtggaaccccgagatccagtacaccagcaactactacaagagcaacaacgtggagttcgccgtgaacaccgagggcgtgtacagcgagcccaggcccatcggcaccaggtacctgaccaggaacctg

SEQ ID NO: 37 - DNA, 2208 bp, synthetic construct
  misc_feature 1..2208: note = Recombinant expression cassette or expression cassette component
  source 1..2208: mol_type = other DNA
atggccgccgacggctacctgcccgactggctggaggacaacctgagcgagggcatcagggagtggtgggccctgaagcccggcgccccccagcccaaggccaaccagcagcaccaggacaacgccaggggcctggtgctgcccggctacaagtacctgggccccggcaacggcctggacaagggcgagcccgtgaacgccgccgacgccgccgccctggagcacgacaaggcctacgaccagcagctgaaggccggcgacaacccctacctgaagtacaaccacgccgacgccgagttccaggagaggctgaaggaggacaccagcttcggcggcaacctgggcagggccgtgttccaggccaagaagaggctgctggagcccctgggcctggtggaggaggccgccaagaccgcccccggcaagaagaggcccgtggagcagagcccccaggagcccgacagcagcgccggcatcggcaagagcggcgcccagcccgccaagaagaggctgaacttcggccagaccggcgacaccgagagcgtgcccgacccccagcccatcggcgagccccccgccgcccccagcggcgtgggcagcctgaccatggccagcggcggcggcgcccccgtggccgacaacaacgagggcgccgacggcgtgggcagcagcagcggcaactggcactgcgacagccagtggctgggcgacagggtgatcaccaccagcaccaggacctgggccctgcccacctacaacaaccacctgtacaagcagatcagcaacagcaccagcggcggcagcagcaacgacaacgcctacttcggctacagcaccccctggggctacttcgacttcaacaggttccactgccacttcagccccagggactggcagaggctgatcaacaacaactggggcttcaggcccaagaggctgaacttcaagctgttcaacatccaggtgaaggaggtgaccgacaacaacggcgtgaagaccatcgccaacaacctgaccagcaccgtgcaggtgttcaccgacagcgactaccagctgccctacgtgctgggcagcgcccacgagggctgcctgccccccttccccgccgacgtgttcatgatcccccagtacggctacctgaccctgaacgacggcagccaggccgtgggcaggagcagcttctactgcctggagtacttccccagccagatgctgaggaccggcaacaacttccagttcagctacgagttcgagaacgtgcccttccacagcagctacgcccacagccagagcctggacaggctgatgaaccccctgatcgaccagtacctgtactacctgagcaagaccatcaacggcagcggccagaaccagcagaccctgaagttcagcgtggccggccccagcaacatggccgtgcagggcaggaactacatccccggccccagctacaggcagcagagggtgagcaccaccgtgacccagaacctgaacagcatgctgatctggcccggcgccagcagctgggccctgaacggcaggaacagcctgatgaaccccggccccgccatggccagccacaaggagggcgaggacaggttcttccccctgagcggcagcctgatcttcggcaagcagggcaccggcagggacaacgtggacgccgacaaggtgatgatcaccaacgaggaggagatcaagaccaccaaccccgtggccaccgagagctacggccaggtggccaccaaccaccagagcgcccaggcccaggcccagaccggctgggtgcagaaccagggcatcctgcccggcatggtgtggcaggacagggacgtgtacctgcagggccccatctgggccaagatcccccacaccgacggcaacttccaccccagccccctgatgggcggcttcggcatgaagcacccccccccccagatcctgatcaagaacacccccgtgcccgccgacccccccaccgccttcaacaaggacaagctgaacagcttcatcacccagtacagcaccggccaggtgagcgtggagatcgagtgggagctgcagaaggagaacagcaagaggtggaaccccgagatccagtacaccagcaactactacaagagcaacaacgtggagttcgccgtgaacaccgagggcgtgtacagcgagcccaggcccatcggcaccaggtacctgaccaggaacctg

SEQ ID NO: 38 - DNA, 2181 bp, synthetic construct
  misc_feature 1..2181: note = Recombinant expression cassette or expression cassette component
  source 1..2181: mol_type = other DNA
atggccgccgacggctacctgcccgactggctggaggacaacctgagcgagggcatcagggagtggtgggccctgaagcccggcgccccccagcccaaggccaaccagcagcaccaggacaacgccaggggcctggtgctgcccggctacaagtacctgggccccggcaacggcctggacaagggcgagcccgtgaacgccgccgacgccgccgccctggagcacgacaaggcctacgaccagcagctgaaggccggcgacaacccctacctgaagtacaaccacgccgacgccgagttccaggagaggctgaaggaggacaccagcttcggcggcaacctgggcagggccgtgttccaggccaagaagaggctgctggagcccttcggcctggtggaggagggcgccaagaccgcccccaccggcaagaggatcgacgaccacttccccaagaggaagaaggccaggaccgaggaggacagcaagcccagcaccagcagcgacgccgaggccggccccagcggcagccagcagctgcagatccccgcccagcccgccagcagcctgggcgccgacaccatgagcgccggcggcggcggccccctgggcgacaacaacgagggcgccgacggcgtgggcagcagcagcggcaactggcactgcgacagccagtggctgggcgacagggtgatcaccaccagcaccaggacctgggccctgcccacctacaacaaccacctgtacaagcagatcagcaacagcaccagcggcggcagcagcaacgacaacgcctacttcggctacagcaccccctggggctacttcgacttcaacaggttccactgccacttcagccccagggactggcagaggctgatcaacaacaactggggcttcaggcccaagaggctgaacttcaagctgttcaacatccaggtgaaggaggtgaccgacaacaacggcgtgaagaccatcgccaacaacctgaccagcaccgtgcaggtgttcaccgacagcgactaccagctgccctacgtgctgggcagcgcccacgagggctgcctgccccccttccccgccgacgtgttcatgatcccccagtacggctacctgaccctgaacgacggcagccaggccgtgggcaggagcagcttctactgcctggagtacttccccagccagatgctgaggaccggcaacaacttccagttcagctacgagttcgagaacgtgcccttccacagcagctacgcccacagccagagcctggacaggctgatgaaccccctgatcgaccagtacctgtactacctgagcaagaccatcggctaccacaagagcggcgccgcccagctgaagttcagcgtggccggccccagcaacatggccgtgcagggcaggaactacatccccggccccagctacaggcagcagagggtgagcaccaccgtgacccagaacaacaacagcgagttcgcctggcccggcgccagcagctgggccctgaacggcaggaacagcctgatgaaccccggccccgccatggccagccacaaggagggcgaggacaggttcttccccctgagcggcagcctgatcttcggcaagcagggcaccggcagggacaacgtggacgccgacaaggtgatgatcaccaacgaggaggagatcaagaccaccaaccccgtggccaccgagagctacggccaggtggccaccaaccaccagagcgcccaggcccaggcccagaccggctgggtgcagaaccagggcatcctgcccggcatggtgtggcaggacagggacgtgtacctgcagggccccatctgggccaagatcccccacaccgacggcaacttccaccccagccccctgatgggcggcttcggcatgaagcacccccccccccagatcctgatcaagaacacccccgtgcccgccgacccccccaccgccttcaacaaggacaagctgaacagcttcatcacccagtacagcaccggccaggtgagcgtggagatcgagtgggagctgcagaaggagaacagcaagaggtggaaccccgagatccagtacaccagcaactactacaagagcaacaacgtggagttcgccgtgaacaccgagggcgtgtacagcgagcccaggcccatcggcaccaggtacctgaccaggaacctg

SEQ ID NO: 39 - protein, 736 aa, synthetic construct
  REGION 1..736: note = Recombinant expression cassette or expression cassette component
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 40 - DNA, 2208 bp, synthetic construct
  misc_feature 1..2208: note = Recombinant expression cassette or expression cassette component
  source 1..2208: mol_type = other DNA
atggccgccgacggctacctgcccgactggctggaggacaacctgagcgagggcatcagggagtggtgggacctgaagcccggcgcccccaagcccaaggccaaccagcagaagcaggacgacggcaggggcctggtgctgcccggctacaagtacctgggccccttcaacggcctggacaagggcgagcccgtgaacgccgccgacgccgccgccctggagcacgacaaggcctacgaccagcagctgaaggccggcgacaacccctacctgaggtacaaccacgccgacgccgagttccaggagaggctgcaggaggacaccagcttcggcggcaacctgggcagggccgtgttccaggccaagaagagggtgctggagcccttcggcctggtggaggagggcgccaagaccgcccccggcaagaagaggcccgtggagcagagcccccaggagcccgacagcagcagcggcatcggcaagaccggccagcagcccgccaagaagaggctgaacttcggccagaccggcgacagcgagagcgtgcccgacccccagcccctgggcgagccccccgccacccccgccgccgtgggccccaccaccatggccagcggcggcggcgcccccatggccgacaacaacgagggcgccgacggcgtgggcaacgccagcggcaactggcactgcgacagcacctggctgggcgacagggtgatcaccaccagcaccaggacctgggccctgcccacctacaacaaccacctgtacaagcagatcagcagcgccagcaccggcgccagcaacgacaaccactacttcggctacagcaccccctggggctacttcgacttcaacaggttccactgccacttcagccccagggactggcagaggctgatcaacaacaactggggcttcaggcccaagaggctgaacttcaagctgttcaacatccaggtgaaggaggtgaccaccaacgacggcgtgaccaccatcgccaacaacctgaccagcaccgtgcaggtgttcagcgacagcgagtaccagctgccctacgtgctgggcagcgcccaccagggctgcctgccccccttccccgccgacgtgttcatgatcccccagtacggctacctgaccctgaacaacggcagccaggccgtgggcaggagcagcttctactgcctggagtacttccccagccagatgctgaggaccggcaacaacttcaccttcagctacaccttcgaggacgtgcccttccacagcagctacgcccacagccagagcctggacaggctgatgaaccccctgatcgaccagtacctgtactacctgaacaggacccagaaccagagcggcagcgcccagaacaaggacctgctgttcagcaggggcagccccgccggcatgagcgtgcagcccaagaactggctgcccggcccctgctacaggcagcagagggtgagcaagaccaagaccgacaacaacaacagcaacttcacctggaccggcgccagcaagtacaacctgaacggcagggagagcatcatcaaccccggcaccgccatggccagccacaaggacgacaaggacaagttcttccccatgagcggcgtgatgatcttcggcaaggagagcgccggcgccagcaacaccgccctggacaacgtgatgatcaccgacgaggaggagatcaaggccaccaaccccgtggccaccgagaggttcggcaccgtggccgtgaacctgcagagcagcagcaccgaccccgccaccggcgacgtgcacgtgatgggcgccctgcccggcatggtgtggcaggacagggacgtgtacctgcagggccccatctgggccaagatcccccacaccgacggccacttccaccccagccccctgatgggcggcttcggcctgaagcacccccccccccagatcctgatcaagaacacccccgtgcccgccaacccccccgccgagttcagcgccaccaagttcgccagcttcatcacccagtacagcaccggccaggtgagcgtggagatcgagtgggagctgcagaaggagaacagcaagaggtggaaccccgaggtgcagtacaccagcaactacgccaagagcgccaacgtggacttcaccgtggacaacaacggcctgtacaccgagcccaggcccatcggcaccaggtacctgaccaggcccctg

SEQ ID NO: 41 - protein, 724 aa, synthetic construct
  REGION 1..724: note = Recombinant expression cassette or expression cassette component
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 42 - DNA, 2172 bp, synthetic construct
  misc_feature 1..2172: note = Recombinant expression cassette or expression cassette component
  source 1..2172: mol_type = other DNA
atgagcttcgtggaccacccccccgactggctggaggaggtgggcgagggcctgagggagttcctgggcctggaggccggcccccccaagcccaagcccaaccagcagcaccaggaccaggccaggggcctggtgctgcccggctacaactacctgggccccggcaacggcctggacaggggcgagcccgtgaacagggccgacgaggtggccagggagcacgacatcagctacaacgagcagctggaggccggcgacaacccctacctgaagtacaaccacgccgacgccgagttccaggagaagctggccgacgacaccagcttcggcggcaacctgggcaaggccgtgttccaggccaagaagagggtgctggagcccttcggcctggtggaggagggcgccaagaccgcccccaccggcaagaggatcgacgaccacttccccaagaggaagaaggccaggaccgaggaggacagcaagcccagcaccagcagcgacgccgaggccggccccagcggcagccagcagctgcagatccccgcccagcccgccagcagcctgggcgccgacaccatgagcgccggcggcggcggccccctgggcgacaacaaccagggcgccgacggcgtgggcaacgccagcggcgactggcactgcgacagcacctggatgggcgacagggtggtgaccaagagcaccaggacctgggtgctgcccagctacaacaaccaccagtacagggagatcaagagcggcagcgtggacggcagcaacgccaacgcctacttcggctacagcaccccctggggctacttcgacttcaacaggttccacagccactggagccccagggactggcagaggctgatcaacaactactggggcttcaggcccaggagcctgagggtgaagatcttcaacatccaggtgaaggaggtgaccgtgcaggacagcaccaccaccatcgccaacaacctgaccagcaccgtgcaggtgttcaccgacgacgactaccagctgccctacgtggtgggcaacggcaccgagggctgcctgcccgccttccccccccaggtgttcaccctgccccagtacggctacgccaccctgaacagggacaacaccgagaaccccaccgagaggagcagcttcttctgcctggagtacttccccagcaagatgctgaggaccggcaacaacttcgagttcacctacaacttcgaggaggtgcccttccacagcagcttcgcccccagccagaacctgttcaagctggccaaccccctggtggaccagtacctgtacaggttcgtgagcaccaacaacaccggcggcgtgcagttcaacaagaacctggccggcaggtacgccaacacctacaagaactggttccccggccccatgggcaggacccagggctggaacctgggcagcggcgtgaacagggccagcgtgagcgccttcgccaccaccaacaggatggagctggagggcgccagctaccaggtgcccccccagcccaacggcatgaccaacaacctgcagggcagcaacacctacgccctggagaacaccatgatcttcaacagccagcccgccaaccccggcaccaccgccacctacctggagggcaacatgctgatcaccagcgagagcgagacccagcccgtgaacagggtggcctacaacgtgggcggccagatggccaccaacaaccagagcagcaccaccgcccccgccaccggcacctacaacctgcaggagatcgtgcccggcagcgtgtggatggagagggacgtgtacctgcagggccccatctgggccaagatccccgagaccggcgcccacttccaccccagccccgccatgggcggcttcggcctgaagcaccccccccccatgatgctgatcaagaacacccccgtgcccggcaacatcaccagcttcagcgacgtgcccgtgagcagcttcatcacccagtacagcaccggccaggtgaccgtggagatggagtgggagctgaagaaggagaacagcaagaggtggaaccccgagatccagtacaccaacaactacaacgacccccagttcgtggacttcgcccccgacagcaccggcgagtacaggaccaccaggcccatcggcaccaggtacctgaccaggcccctg

SEQ ID NO: 43 - protein, 34 aa, synthetic construct
  REGION 1..34: note = Recombinant expression cassette or expression cassette component
MAEKGSTFSHLLVPILLLIGWIVGCIIMIYVVFS

SEQ ID NO: 44 - DNA, 102 bp, synthetic construct
  misc_feature 1..102: note = Recombinant expression cassette or expression cassette component
  source 1..102: mol_type = other DNA
atggccgagaagggcagcaccttcagccacctgctggtgcccatcctgctgctgatcggctggatcgtgggctgcatcatcatgatctacgtggtgttcagc

SEQ ID NO: 45 - DNA, 2322 bp, synthetic construct
  misc_feature 1..2322: note = Recombinant expression cassette or expression cassette component
  source 1..2322: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagaactggcctgcccgagaccaaacgtgcggaacgtagttaagtgttagaggtaggatttgaagcctgtcgatcattctgattctccttttctctacgtctgcttcctgtcaatgggcatcctcactgtcaaatgcagatggtacagcagggcttggtctcagccaggcaggcctctccccagtctccatggctcagctgtccagcagtttcatccctagaccatcccaaacatggttgagaagctctgaggggaggacccagcactgcccggcccctgaagataatcagcagtcctgctcagcatatcaatccaagcccactctagacagagatgccggtgcccagttttctatttttaactggtgtgaactgaaggaaaagcacagcattagaagtccaagcagtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagcggccgcccgccaccatggctgagaaagagtcaacatcaccacacctcatggttcccattcttctcctggttggatggattgtaggctgcatcatcgttatttacattgtcttcttctaaaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 46 - DNA, 2513 bp, synthetic construct
  misc_feature 1..2513: note = Recombinant expression cassette or expression cassette component
  source 1..2513: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagaactggcctgcccgagaccaaacgtgcggaacgtagttaagtgttagaggtaggatttgaagcctgtcgatcattctgattctccttttctctacgtctgcttcctgtcaatgggcatcctcactgtcaaatgcagatggtacagcagggcttggtctcagccaggcaggcctctccccagtctccatggctcagctgtccagcagtttcatccctagaccatcccaaacatggttgagaagctctgaggggaggacccagcactgcccggcccctgaagataatcagcagtcctgctcagcatatcaatccaagcccactctagacagagatgccggtgcccagttttctatttttaactggtgtgaactgaaggaaaagcacagcattagaagtccaagcaccttcagattaaaaataactaaggtaagggccatgtgggtaggggaggtggtgtgagacggtcctgtctctcctctatctgcccatcggccctttggggaggaggaatgtgcccaaggactaaaaaaaggccctggagccagaggggcgagggcagcagacctttcatgggcaaacctcagggctgctgtcgtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagcggccgcccgccaccatggctgagaaagagtcaacatcaccacacctcatggttcccattcttctcctggttggatggattgtaggctgcatcatcgttatttacattgtcttcttctaaaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 47 - DNA, 2110 bp, synthetic construct
  misc_feature 1..2110: note = Recombinant expression cassette or expression cassette component
  source 1..2110: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagccttcagattaaaaataactaaggtaagggccatgtgggtaggggaggtggtgtgagacggtcctgtctctcctctatctgcccatcggccctttggggaggaggaatgtgcccaaggactaaaaaaaggccctggagccagaggggcgagggcagcagacctttcatgggcaaacctcagggctgctgtcgtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagcggccgcccgccaccatggctgagaaagagtcaacatcaccacacctcatggttcccattcttctcctggttggatggattgtaggctgcatcatcgttatttacattgtcttcttctaaaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 48 - DNA, 2513 bp, synthetic construct
  misc_feature 1..2513: note = Recombinant expression cassette or expression cassette component
  source 1..2513: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagccttcagattaaaaataactaaggtaagggccatgtgggtaggggaggtggtgtgagacggtcctgtctctcctctatctgcccatcggccctttggggaggaggaatgtgcccaaggactaaaaaaaggccctggagccagaggggcgagggcagcagacctttcatgggcaaacctcagggctgctgtcaactggcctgcccgagaccaaacgtgcggaacgtagttaagtgttagaggtaggatttgaagcctgtcgatcattctgattctccttttctctacgtctgcttcctgtcaatgggcatcctcactgtcaaatgcagatggtacagcagggcttggtctcagccaggcaggcctctccccagtctccatggctcagctgtccagcagtttcatccctagaccatcccaaacatggttgagaagctctgaggggaggacccagcactgcccggcccctgaagataatcagcagtcctgctcagcatatcaatccaagcccactctagacagagatgccggtgcccagttttctatttttaactggtgtgaactgaaggaaaagcacagcattagaagtccaagcagtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagcggccgcccgccaccatggctgagaaagagtcaacatcaccacacctcatggttcccattcttctcctggttggatggattgtaggctgcatcatcgttatttacattgtcttcttctaaaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 49 - DNA, 2691 bp, synthetic construct
  misc_feature 1..2691: note = Recombinant expression cassette or expression cassette component
  source 1..2691: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagccttcagattaaaaataactaaggtaagggccatgtgggtaggggaggtggtgtgagacggtcctgtctctcctctatctgcccatcggccctttggggaggaggaatgtgcccaaggactaaaaaaaggccctggagccagaggggcgagggcagcagacctttcatgggcaaacctcagggctgctgtcaactggcctgcccgagaccaaacgtgcggaacgtagttaagtgttagaggtaggatttgaagcctgtcgatcattctgattctccttttctctacgtctgcttcctgtcaatgggcatcctcactgtcaaatgcagatggtacagcagggcttggtctcagccaggcaggcctctccccagtctccatggctcagctgtccagcagtttcatccctagaccatcccaaacatggttgagaagctctgaggggaggacccagcactgcccggcccctgaagataatcagcagtcctgctcagcatatcaatccaagcccactctagacagagatgccggtgcccagttttctatttttaactggtgtgaactgaaggaaaagcacagcattagaagtccaagcagtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccataactggtaagtaccgcctatagactctataggcacacccctttggctcttatgcatgctgacagactaacagactgttcctttcctgggtcttttctgcaggcctgtacggaagtgttacttctgctctaaaagctgcggaattgtacccgcggccgatccaatcgatacagatctagcggccgcccgccaccatggctgagaaagagtcaacatcaccacacctcatggttcccattcttctcctggttggatggattgtaggctgcatcatcgttatttacattgtcttcttctaaaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 50 - DNA, 2097 bp, synthetic construct
  misc_feature 1..2097: note = Recombinant expression cassette or expression cassette component
  source 1..2097: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaaggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccataactggtaagtaccgcctatagactctataggcacacccctttggctcttatgcatgctgacagactaacagactgttcctttcctgggtcttttctgcaggcctgtacggaagtgttacttctgctctaaaagctgcggaattgtacccgcggccgatccaatcgatacagatctagcggccgcccgccaccatggctgagaaagagtcaacatcaccacacctcatggttcccattcttctcctggttggatggattgtaggctgcatcatcgttatttacattgtcttcttctaaaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 51 - DNA, 2691 bp, synthetic construct
  misc_feature 1..2691: note = Recombinant expression cassette or expression cassette component
  source 1..2691: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagaactggcctgcccgagaccaaacgtgcggaacgtagttaagtgttagaggtaggatttgaagcctgtcgatcattctgattctccttttctctacgtctgcttcctgtcaatgggcatcctcactgtcaaatgcagatggtacagcagggcttggtctcagccaggcaggcctctccccagtctccatggctcagctgtccagcagtttcatccctagaccatcccaaacatggttgagaagctctgaggggaggacccagcactgcccggcccctgaagataatcagcagtcctgctcagcatatcaatccaagcccactctagacagagatgccggtgcccagttttctatttttaactggtgtgaactgaaggaaaagcacagcattagaagtccaagcaccttcagattaaaaataactaaggtaagggccatgtgggtaggggaggtggtgtgagacggtcctgtctctcctctatctgcccatcggccctttggggaggaggaatgtgcccaaggactaaaaaaaggccctggagccagaggggcgagggcagcagacctttcatgggcaaacctcagggctgctgtcgtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccataactggtaagtaccgcctatagactctataggcacacccctttggctcttatgcatgctgacagactaacagactgttcctttcctgggtcttttctgcaggcctgtacggaagtgttacttctgctctaaaagctgcggaattgtacccgcggccgatccaatcgatacagatctagcggccgcccgccaccatggccgagaaggaatctaccagcccccacctgatggtgcctattctgctgctggtgggctggatcgtcggctgcatcatcgtgatctacatcgtgttcttctgaaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 52 - DNA, 2500 bp, synthetic construct
  misc_feature 1..2500: note = Recombinant expression cassette or expression cassette component
  source 1..2500: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagaactggcctgcccgagaccaaacgtgcggaacgtagttaagtgttagaggtaggatttgaagcctgtcgatcattctgattctccttttctctacgtctgcttcctgtcaatgggcatcctcactgtcaaatgcagatggtacagcagggcttggtctcagccaggcaggcctctccccagtctccatggctcagctgtccagcagtttcatccctagaccatcccaaacatggttgagaagctctgaggggaggacccagcactgcccggcccctgaagataatcagcagtcctgctcagcatatcaatccaagcccactctagacagagatgccggtgcccagttttctatttttaactggtgtgaactgaaggaaaagcacagcattagaagtccaagcagtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccataactggtaagtaccgcctatagactctataggcacacccctttggctcttatgcatgctgacagactaacagactgttcctttcctgggtcttttctgcaggcctgtacggaagtgttacttctgctctaaaagctgcggaattgtacccgcggccgatccaatcgatacagatctagcggccgcccgccaccatggctgagaaagagtcaacatcaccacacctcatggttcccattcttctcctggttggatggattgtaggctgcatcatcgttatttacattgtcttcttctaaaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 53 - DNA, 2288 bp, synthetic construct
  misc_feature 1..2288: note = Recombinant expression cassette or expression cassette component
  source 1..2288: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagccttcagattaaaaataactaaggtaagggccatgtgggtaggggaggtggtgtgagacggtcctgtctctcctctatctgcccatcggccctttggggaggaggaatgtgcccaaggactaaaaaaaggccctggagccagaggggcgagggcagcagacctttcatgggcaaacctcagggctgctgtcgtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccataactggtaagtaccgcctatagactctataggcacacccctttggctcttatgcatgctgacagactaacagactgttcctttcctgggtcttttctgcaggcctgtacggaagtgttacttctgctctaaaagctgcggaattgtacccgcggccgatccaatcgatacagatctagcggccgcccgccaccatggctgagaaagagtcaacatcaccacacctcatggttcccattcttctcctggttggatggattgtaggctgcatcatcgttatttacattgtcttcttctaaaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 54 - DNA, 1919 bp, synthetic construct
  misc_feature 1..1919: note = Recombinant expression cassette or expression cassette component
  source 1..1919: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaaggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagcggccgcccgccaccatggccgagaaggaatctaccagcccccacctgatggtgcctattctgctgctggtgggctggatcgtcggctgcatcatcgtgatctacatcgtgttcttctgaaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 55 - DNA, 3865 bp, synthetic construct
  misc_feature 1..3865: note = Recombinant expression cassette or expression cassette component
  source 1..3865: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagccttcagattaaaaataactaaggtaagggccatgtgggtaggggaggtggtgtgagacggtcctgtctctcctctatctgcccatcggccctttggggaggaggaatgtgcccaaggactaaaaaaaggccctggagccagaggggcgagggcagcagacctttcatgggcaaacctcagggctgctgtcgtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccataactggtaagtaccgcctatagactctataggcacacccctttggctcttatgcatgctgacagactaacagactgttcctttcctgggtcttttctgcaggcctgtacggaagtgttacttctgctctaaaagctgcggaattgtacccgcggccgatccaatcgatacagatctagcggccgcccgccaccatggctgagaaagagtcaacatcaccacacctcatggttcccattcttctcctggttggatggattgtaggctgcatcatcgttatttacattgtcttcttctaaaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcagccagaagtcagatgctcaaggggcttcatgatgtccccataatttttggcagagggaaaaagatcggatcctcaggcgtagttcaccccgtcctcgaggccgcccgggtcgactaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccgcgcggccgtcagaagaacacgatgtagatcacgatgatgcagccgacgatccagcccaccagcagcagaataggcaccatcaggtgggggctggtagattccttctcggccatggtggcggctagcctatagtgagtcgtattaagtactctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttctgcagcttaagttcgagactgttgtgtcagaagcactgactgcgttagcaatttaactgtgataaactaccgcaataaagctctagagcttcggggatcgtcccacggagcggtgggtgccggcggctgtctgggaagggctccttggggggcagaggctttaaggtccccccggcgcccaccccggggcgggcagagccagcaggaatgtgcccggcgcccagagaggaatgcaacacttgtgagctgctattttggcagcagcggccccggccccctccgtgctcccccttcccccacaggagcccatataagcccaagctattgtgtggcctcagagttttgctattttaaacccgtcggacggagatacgtgagtgcccgaggggctgacacaagccagccagctgtcacctcccagggctggggacgctgataaggcagcgcttcggacccgaccctctgccgcagccccagatgctgtcatgtgaaagcccagactgcttttatccctgcttggacttctaatgctgtgcttttccttcagttcacaccagttaaaaatagaaaactgggcaccggcatctctgtctagagtgggcttggattgatatgctgagcaggactgctgattatcttcaggggccgggcagtgctgggtcctcccctcagagcttctcaaccatgtttgggatggtctagggatgaaactgctggacagctgagccatggagactggggagaggcctgcctggctgagaccaagccctgctgtaccatctgcatttgacagtgaggatgcccattgacaggaagcagacgtagagaaaaggagaatcagaatgatcgacaggcttcaaatcctacctctaacacttaactacgttccgcacgtttggtctcgggcaggccagttgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 56 - DNA, 3865 bp, synthetic construct
  misc_feature 1..3865: note = Recombinant expression cassette or expression cassette component
  source 1..3865: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagccttcagattaaaaataactaaggtaagggccatgtgggtaggggaggtggtgtgagacggtcctgtctctcctctatctgcccatcggccctttggggaggaggaatgtgcccaaggactaaaaaaaggccctggagccagaggggcgagggcagcagacctttcatgggcaaacctcagggctgctgtcgtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccataactggtaagtaccgcctatagactctataggcacacccctttggctcttatgcatgctgacagactaacagactgttcctttcctgggtcttttctgcaggcctgtacggaagtgttacttctgctctaaaagctgcggaattgtacccgcggccgatccaatcgatacagatctagcggccgcccgccaccatggctgagaaagagtcaacatcaccacacctcatggttcccattcttctcctggttggatggattgtaggctgcatcatcgttatttacattgtcttcttctaaaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcagccagaagtcagatgctcaaggggcttcatgatgtccccataatttttggcagagggaaaaagatcggatcctcaggcgtagttcaccccgtcctcgaggccgcccgggtcgactaaaaaacctcccacacctccccctgaacctgaaacataaaatgaatgcaattgttgttgttaaactggcctgcccgagaccaaacgtgcggaacgtagttaagtgttagaggtaggatttgaagcctgtcgatcattctgattctccttttctctacgtctgcttcctgtcaatgggcatcctcactgtcaaatgcagatggtacagcagggcttggtctcagccaggcaggcctctccccagtctccatggctcagctgtccagcagtttcatccctagaccatcccaaacatggttgagaagctctgaggggaggacccagcactgcccggcccctgaagataatcagcagtcctgctcagcatatcaatccaagcccactctagacagagatgccggtgcccagttttctatttttaactggtgtgaactgaaggaaaagcacagcattagaagtccaagcagggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacgatccccgaagctctagagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccgccaccatggccgagaaggaatctaccagcccccacctgatggtgcctattctgctgctggtgggctggatcgtcggctgcatcatcgtgatctacatcgtgttcttctgacggccgcgcggatccagacatgataagatacattgatgagtttggacaaaccacaactagaatgcagtgaaaaaaatgctttatttgtgaaatttgtgatgctattgctttatttgtaaccattataagctgcaataaacaagtgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 57 - DNA, 3856 bp, synthetic construct
  misc_feature 1..3856: note = Recombinant expression cassette or expression cassette component
  source 1..3856: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggcagatctcgaagacgcggaagaggccgcagagccggcagcaggccgcgggaaggaaggtccgctggattgagggccgaagggacgtagcagaaggacgtcccgcgcagaatccaggtggcaacacaggcgagcagccaaggaaaggacgatgatttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattggatccaaagcttttagaagaagacaatgtaaataacgatgatgcagcctacaatccatccaaccaggagaagaatgggaaccatgaggtgtggtgatgttgactctttctcagccatggtggcgggcggccgctagatctgtatcgattggatcggccgcgggtacaattccgcagcttttagagcagaagtaacacttccgtacaggcctgcagaaaagacccaggaaaggaacagtctgttagtctgtcagcatgcataagagccaaaggggtgtgcctatagagtctataggcggtacttaccagttatggaggcgtctgctcagtctcagcggggactgggtgaggcagaggatggagagggctttaagcaggcatgtgggctggggcctggtgagccagccctgcggagggaggaatgtgcgacaggggacgggtggggcagggggatggcggtgggggtggggggtgttggctgctattttggcaggtgccagggacaaggctacaggaacatgtaccccacgccatataagcccatgtggtcctccagctgctcagataagctatttaaaaccagagcagatatgcagggaacagtcatgcaacataaaccagctgtccctcttgagaatcctgataaagcagaggccagcaacccaggcctgggagggccagctgggagcagggttggggggcagaaggcaacctccaagacactccataagtctcagcaccagaatcttggaaggcagagggcaagagttatgtgctgctccacttgaactgatgctgggggtaaagacatcttccaggctactggctcctaatggactgagcagccttaggcaggttgccggctctgccagccccagtgaggacatctgcaaggtgggtcttctccatgacgacagcagccctgaggtttgcccatgaaaggtctgctgccctcgcccctctggctccagggcctttttttagtccttgggcacattcctcctccccaaagggccgatgggcagatagaggagagacaggaccgtctcacaccacctcccctacccacatggcccttaccttagttatttttaatctgaaggctcgagttaagggcagccagaagtcagatgctcaaggggcttcatgatgtccccataatttttggcagagggaaaaagatcggatcctcaggcgtagttcaccccgtcctcgaggccgcccgggtcgactaaaaaacctcccacacctccccctgaacctgaaacataaaatgaatgcaattgttgttgttaaactggcctgcccgagaccaaacgtgcggaacgtagttaagtgttagaggtaggatttgaagcctgtcgatcattctgattctccttttctctacgtctgcttcctgtcaatgggcatcctcactgtcaaatgcagatggtacagcagggcttggtctcagccaggcaggcctctccccagtctccatggctcagctgtccagcagtttcatccctagaccatcccaaacatggttgagaagctctgaggggaggacccagcactgcccggcccctgaagataatcagcagtcctgctcagcatatcaatccaagcccactctagacagagatgccggtgcccagttttctatttttaactggtgtgaactgaaggaaaagcacagcattagaagtccaagcagggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacgatccccgaagctctagagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccgccaccatggccgagaaggaatctaccagcccccacctgatggtgcctattctgctgctggtgggctggatcgtcggctgcatcatcgtgatctacatcgtgttcttctgacggccgcgcggatccagacatgataagatacattgatgagtttggacaaaccacaactagaatgcagtgaaaaaaatgctttatttgtgaaatttgtgatgctattgctttatttgtaaccattataagctgcaataaacaagtgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 58 - DNA, 3271 bp, synthetic construct
  misc_feature 1..3271: note = Recombinant expression cassette or expression cassette component
  source 1..3271: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaaggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccataactggtaagtaccgcctatagactctataggcacacccctttggctcttatgcatgctgacagactaacagactgttcctttcctgggtcttttctgcaggcctgtacggaagtgttacttctgctctaaaagctgcggaattgtacccgcggccgatccaatcgatacagatctagcggccgcccgccaccatggctgagaaagagtcaacatcaccacacctcatggttcccattcttctcctggttggatggattgtaggctgcatcatcgttatttacattgtcttcttctaaaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcagccagaagtcagatgctcaaggggcttcatgatgtccccataatttttggcagagggaaaaagatcggatcctcaggcgtagttcaccccgtcctcgaggccgcccgggtcgactaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccgcgcggccgtcagaagaacacgatgtagatcacgatgatgcagccgacgatccagcccaccagcagcagaataggcaccatcaggtgggggctggtagattccttctcggccatggtggcggctagcctatagtgagtcgtattaagtactctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttctgcagcttaagttcgagactgttgtgtcagaagcactgactgcgttagcaatttaactgtgataaactaccgcaataaagctctagagcttcggggatcgtcccacggagcggtgggtgccggcggctgtctgggaagggctccttggggggcagaggctttaaggtccccccggcgcccaccccggggcgggcagagccagcaggaatgtgcccggcgcccagagaggaatgcaacacttgtgagctgctattttggcagcagcggccccggccccctccgtgctcccccttcccccacaggagcccatataagcccaagctattgtgtggcctcagagttttgctattttaaacccgtcggacggagatacgtgagtgcccgaggggctgacacaagccagccagctgtcacctcccagggctggggacgctgataaggcagcgcttcggacccgaccctctgccgcagccccagatgctgtcatgtgaaagcccagactgcttttatcccgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 59 - DNA, 3271 bp, synthetic construct
  misc_feature 1..3271: note = Recombinant expression cassette or expression cassette component
  source 1..3271: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaaggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccataactggtaagtaccgcctatagactctataggcacacccctttggctcttatgcatgctgacagactaacagactgttcctttcctgggtcttttctgcaggcctgtacggaagtgttacttctgctctaaaagctgcggaattgtacccgcggccgatccaatcgatacagatctagcggccgcccgccaccatggctgagaaagagtcaacatcaccacacctcatggttcccattcttctcctggttggatggattgtaggctgcatcatcgttatttacattgtcttcttctaaaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcagccagaagtcagatgctcaaggggcttcatgatgtccccataatttttggcagagggaaaaagatcggatcctcaggcgtagttcaccccgtcctcgaggccgcccgggtcgactaaaaaacctcccacacctccccctgaacctgaaacataaaatgaatgcaattgttgttgttagggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacgatccccgaagctctagagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccgccaccatggccgagaaggaatctaccagcccccacctgatggtgcctattctgctgctggtgggctggatcgtcggctgcatcatcgtgatctacatcgtgttcttctgacggccgcgcggatccagacatgataagatacattgatgagtttggacaaaccacaactagaatgcagtgaaaaaaatgctttatttgtgaaatttgtgatgctattgctttatttgtaaccattataagctgcaataaacaagtgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 60 - DNA, 3262 bp, synthetic construct
  misc_feature 1..3262: note = Recombinant expression cassette or expression cassette component
  source 1..3262: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggcagatctcgaagacgcggaagaggccgcagagccggcagcaggccgcgggaaggaaggtccgctggattgagggccgaagggacgtagcagaaggacgtcccgcgcagaatccaggtggcaacacaggcgagcagccaaggaaaggacgatgatttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattggatccaaagcttttagaagaagacaatgtaaataacgatgatgcagcctacaatccatccaaccaggagaagaatgggaaccatgaggtgtggtgatgttgactctttctcagccatggtggcgggcggccgctagatctgtatcgattggatcggccgcgggtacaattccgcagcttttagagcagaagtaacacttccgtacaggcctgcagaaaagacccaggaaaggaacagtctgttagtctgtcagcatgcataagagccaaaggggtgtgcctatagagtctataggcggtacttaccagttatggaggcgtctgctcagtctcagcggggactgggtgaggcagaggatggagagggctttaagcaggcatgtgggctggggcctggtgagccagccctgcggagggaggaatgtgcgacaggggacgggtggggcagggggatggcggtgggggtggggggtgttggctgctattttggcaggtgccagggacaaggctacaggaacatgtaccccacgccatataagcccatgtggtcctccagctgctcagataagctatttaaaaccagagcagatatgcagggaacagtcatgcaacataaaccagctgtccctcttgagaatcctgataaagcagaggccagcaacccaggcctgggagggccagctgggagcagggttggggggcagaaggcaacctccaagacactccataagtctcagcaccagaatcttggaaggcagagggcaagagttatgtgctgctccacttgaactgatgctgggggtaaagacatcttccaggctactggctcctaatggactgagcagccttaggcaggttgccggctctgccagccccagtgaggacatctgcaaggtgggtcttctccatgacctcgagttaagggcagccagaagtcagatgctcaaggggcttcatgatgtccccataatttttggcagagggaaaaagatcggatcctcaggcgtagttcaccccgtcctcgaggccgcccgggtcgactaaaaaacctcccacacctccccctgaacctgaaacataaaatgaatgcaattgttgttgttagggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacgatccccgaagctctagagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccgccaccatggccgagaaggaatctaccagcccccacctgatggtgcctattctgctgctggtgggctggatcgtcggctgcatcatcgtgatctacatcgtgttcttctgacggccgcgcggatccagacatgataagatacattgatgagtttggacaaaccacaactagaatgcagtgaaaaaaatgctttatttgtgaaatttgtgatgctattgctttatttgtaaccattataagctgcaataaacaagtgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 61 - DNA, 2325 bp, synthetic construct
  misc_feature 1..2325: note = Recombinant expression cassette or expression cassette component
  source 1..2325: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagaactggcctgcccgagaccaaacgtgcggaacgtagttaagtgttagaggtaggatttgaagcctgtcgatcattctgattctccttttctctacgtctgcttcctgtcaatgggcatcctcactgtcaaatgcagatggtacagcagggcttggtctcagccaggcaggcctctccccagtctccatggctcagctgtccagcagtttcatccctagaccatcccaaacatggttgagaagctctgaggggaggacccagcactgcccggcccctgaagataatcagcagtcctgctcagcatatcaatccaagcccactctagacagagatgccggtgcccagttttctatttttaactggtgtgaactgaaggaaaagcacagcattagaagtccaagcagtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagcggccgcccgccaccatggctgaaaaagcggggtctacattttcacaccttctggttcctattcttctcctgattggctggattgtgggctgcatcataatgatttatgttgtcttctcttagaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 62 - DNA, 3277 bp, synthetic construct
  misc_feature 1..3277: note = Recombinant expression cassette or expression cassette component
  source 1..3277: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaaggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccataactggtaagtaccgcctatagactctataggcacacccctttggctcttatgcatgctgacagactaacagactgttcctttcctgggtcttttctgcaggcctgtacggaagtgttacttctgctctaaaagctgcggaattgtacccgcggccgatccaatcgatacagatctagcggccgcccgccaccatggctgaaaaagcggggtctacattttcacaccttctggttcctattcttctcctgattggctggattgtgggctgcatcataatgatttatgttgtcttctcttagaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcagccagaagtcagatgctcaaggggcttcatgatgtccccataatttttggcagagggaaaaagatcggatcctcaggcgtagttcaccccgtcctcgaggccgcccgggtcgactaaaaaacctcccacacctccccctgaacctgaaacataaaatgaatgcaattgttgttgttagggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacgatccccgaagctctagagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccgccaccatggccgagaaggccggatctaccttcagccacctgctggtccctattctgctgctgatcggctggatcgtgggctgcatcatcatgatctacgtggtgttcagctgacggccgcgcggatccagacatgataagatacattgatgagtttggacaaaccacaactagaatgcagtgaaaaaaatgctttatttgtgaaatttgtgatgctattgctttatttgtaaccattataagctgcaataaacaagtgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 63 - DNA, 3268 bp, synthetic construct
  misc_feature 1..3268: note = Recombinant expression cassette or expression cassette component
  source 1..3268: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggcagatctcgaagacgcggaagaggccgcagagccggcagcaggccgcgggaaggaaggtccgctggattgagggccgaagggacgtagcagaaggacgtcccgcgcagaatccaggtggcaacacaggcgagcagccaaggaaaggacgatgatttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattggatccaaagcttctaagagaagacaacataaatcattatgatgcagcccacaatccagccaatcaggagaagaataggaaccagaaggtgtgaaaatgtagaccccgctttttcagccatggtggcgggcggccgctagatctgtatcgattggatcggccgcgggtacaattccgcagcttttagagcagaagtaacacttccgtacaggcctgcagaaaagacccaggaaaggaacagtctgttagtctgtcagcatgcataagagccaaaggggtgtgcctatagagtctataggcggtacttaccagttatggaggcgtctgctcagtctcagcggggactgggtgaggcagaggatggagagggctttaagcaggcatgtgggctggggcctggtgagccagccctgcggagggaggaatgtgcgacaggggacgggtggggcagggggatggcggtgggggtggggggtgttggctgctattttggcaggtgccagggacaaggctacaggaacatgtaccccacgccatataagcccatgtggtcctccagctgctcagataagctatttaaaaccagagcagatatgcagggaacagtcatgcaacataaaccagctgtccctcttgagaatcctgataaagcagaggccagcaacccaggcctgggagggccagctgggagcagggttggggggcagaaggcaacctccaagacactccataagtctcagcaccagaatcttggaaggcagagggcaagagttatgtgctgctccacttgaactgatgctgggggtaaagacatcttccaggctactggctcctaatggactgagcagccttaggcaggttgccggctctgccagccccagtgaggacatctgcaaggtgggtcttctccatgacctcgagttaagggcagccagaagtcagatgctcaaggggcttcatgatgtccccataatttttggcagagggaaaaagatcggatcctcaggcgtagttcaccccgtcctcgaggccgcccgggtcgactaaaaaacctcccacacctccccctgaacctgaaacataaaatgaatgcaattgttgttgttagggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacgatccccgaagctctagagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccgccaccatggccgagaaggccggatctaccttcagccacctgctggtccctattctgctgctgatcggctggatcgtgggctgcatcatcatgatctacgtggtgttcagctgacggccgcgcggatccagacatgataagatacattgatgagtttggacaaaccacaactagaatgcagtgaaaaaaatgctttatttgtgaaatttgtgatgctattgctttatttgtaaccattataagctgcaataaacaagtgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 64 - DNA, 2516 bp, synthetic construct
  misc_feature 1..2516: note = Recombinant expression cassette or expression cassette component
  source 1..2516: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagaactggcctgcccgagaccaaacgtgcggaacgtagttaagtgttagaggtaggatttgaagcctgtcgatcattctgattctccttttctctacgtctgcttcctgtcaatgggcatcctcactgtcaaatgcagatggtacagcagggcttggtctcagccaggcaggcctctccccagtctccatggctcagctgtccagcagtttcatccctagaccatcccaaacatggttgagaagctctgaggggaggacccagcactgcccggcccctgaagataatcagcagtcctgctcagcatatcaatccaagcccactctagacagagatgccggtgcccagttttctatttttaactggtgtgaactgaaggaaaagcacagcattagaagtccaagcaccttcagattaaaaataactaaggtaagggccatgtgggtaggggaggtggtgtgagacggtcctgtctctcctctatctgcccatcggccctttggggaggaggaatgtgcccaaggactaaaaaaaggccctggagccagaggggcgagggcagcagacctttcatgggcaaacctcagggctgctgtcgtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagcggccgcccgccaccatggctgaaaaagcggggtctacattttcacaccttctggttcctattcttctcctgattggctggattgtgggctgcatcataatgatttatgttgtcttctcttagaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 65 - DNA, 2113 bp, synthetic construct
  misc_feature 1..2113: note = Recombinant expression cassette or expression cassette component
  source 1..2113: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagccttcagattaaaaataactaaggtaagggccatgtgggtaggggaggtggtgtgagacggtcctgtctctcctctatctgcccatcggccctttggggaggaggaatgtgcccaaggactaaaaaaaggccctggagccagaggggcgagggcagcagacctttcatgggcaaacctcagggctgctgtcgtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagcggccgcccgccaccatggctgaaaaagcggggtctacattttcacaccttctggttcctattcttctcctgattggctggattgtgggctgcatcataatgatttatgttgtcttctcttagaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 66 - DNA, 2516 bp, synthetic construct
  misc_feature 1..2516: note = Recombinant expression cassette or expression cassette component
  source 1..2516: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagccttcagattaaaaataactaaggtaagggccatgtgggtaggggaggtggtgtgagacggtcctgtctctcctctatctgcccatcggccctttggggaggaggaatgtgcccaaggactaaaaaaaggccctggagccagaggggcgagggcagcagacctttcatgggcaaacctcagggctgctgtcaactggcctgcccgagaccaaacgtgcggaacgtagttaagtgttagaggtaggatttgaagcctgtcgatcattctgattctccttttctctacgtctgcttcctgtcaatgggcatcctcactgtcaaatgcagatggtacagcagggcttggtctcagccaggcaggcctctccccagtctccatggctcagctgtccagcagtttcatccctagaccatcccaaacatggttgagaagctctgaggggaggacccagcactgcccggcccctgaagataatcagcagtcctgctcagcatatcaatccaagcccactctagacagagatgccggtgcccagttttctatttttaactggtgtgaactgaaggaaaagcacagcattagaagtccaagcagtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagcggccgcccgccaccatggctgaaaaagcggggtctacattttcacaccttctggttcctattcttctcctgattggctggattgtgggctgcatcataatgatttatgttgtcttctcttagaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 67 - DNA, 2694 bp, synthetic construct
  misc_feature 1..2694: note = Recombinant expression cassette or expression cassette component
  source 1..2694: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagccttcagattaaaaataactaaggtaagggccatgtgggtaggggaggtggtgtgagacggtcctgtctctcctctatctgcccatcggccctttggggaggaggaatgtgcccaaggactaaaaaaaggccctggagccagaggggcgagggcagcagacctttcatgggcaaacctcagggctgctgtcaactggcctgcccgagaccaaacgtgcggaacgtagttaagtgttagaggtaggatttgaagcctgtcgatcattctgattctccttttctctacgtctgcttcctgtcaatgggcatcctcactgtcaaatgcagatggtacagcagggcttggtctcagccaggcaggcctctccccagtctccatggctcagctgtccagcagtttcatccctagaccatcccaaacatggttgagaagctctgaggggaggacccagcactgcccggcccctgaagataatcagcagtcctgctcagcatatcaatccaagcccactctagacagagatgccggtgcccagttttctatttttaactggtgtgaactgaaggaaaagcacagcattagaagtccaagcagtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccataactggtaagtaccgcctatagactctataggcacacccctttggctcttatgcatgctgacagactaacagactgttcctttcctgggtcttttctgcaggcctgtacggaagtgttacttctgctctaaaagctgcggaattgtacccgcggccgatccaatcgatacagatctagcggccgcccgccaccatggctgaaaaagcggggtctacattttcacaccttctggttcctattcttctcctgattggctggattgtgggctgcatcataatgatttatgttgtcttctcttagaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 68 - DNA, 2100 bp, synthetic construct
  misc_feature 1..2100: note = Recombinant expression cassette or expression cassette component
  source 1..2100: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaaggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccataactggtaagtaccgcctatagactctataggcacacccctttggctcttatgcatgctgacagactaacagactgttcctttcctgggtcttttctgcaggcctgtacggaagtgttacttctgctctaaaagctgcggaattgtacccgcggccgatccaatcgatacagatctagcggccgcccgccaccatggctgaaaaagcggggtctacattttcacaccttctggttcctattcttctcctgattggctggattgtgggctgcatcataatgatttatgttgtcttctcttagaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 69 - DNA, 2694 bp, synthetic construct
  misc_feature 1..2694: note = Recombinant expression cassette or expression cassette component
  source 1..2694: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagaactggcctgcccgagaccaaacgtgcggaacgtagttaagtgttagaggtaggatttgaagcctgtcgatcattctgattctccttttctctacgtctgcttcctgtcaatgggcatcctcactgtcaaatgcagatggtacagcagggcttggtctcagccaggcaggcctctccccagtctccatggctcagctgtccagcagtttcatccctagaccatcccaaacatggttgagaagctctgaggggaggacccagcactgcccggcccctgaagataatcagcagtcctgctcagcatatcaatccaagcccactctagacagagatgccggtgcccagttttctatttttaactggtgtgaactgaaggaaaagcacagcattagaagtccaagcaccttcagattaaaaataactaaggtaagggccatgtgggtaggggaggtggtgtgagacggtcctgtctctcctctatctgcccatcggccctttggggaggaggaatgtgcccaaggactaaaaaaaggccctggagccagaggggcgagggcagcagacctttcatgggcaaacctcagggctgctgtcgtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccataactggtaagtaccgcctatagactctataggcacacccctttggctcttatgcatgctgacagactaacagactgttcctttcctgggtcttttctgcaggcctgtacggaagtgttacttctgctctaaaagctgcggaattgtacccgcggccgatccaatcgatacagatctagcggccgcccgccaccatggctgaaaaagcggggtctacattttcacaccttctggttcctattcttctcctgattggctggattgtgggctgcatcataatgatttatgttgtcttctcttagaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 70 - DNA, 2694 bp, synthetic construct; identical to SEQ ID NO: 69
  misc_feature 1..2694: note = Recombinant expression cassette or expression cassette component
  source 1..2694: mol_type = other DNA

SEQ ID NO: 71 - DNA, 2291 bp, synthetic construct
  misc_feature 1..2291: note = Recombinant expression cassette or expression cassette component
  source 1..2291: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagccttcagattaaaaataactaaggtaagggccatgtgggtaggggaggtggtgtgagacggtcctgtctctcctctatctgcccatcggccctttggggaggaggaatgtgcccaaggactaaaaaaaggccctggagccagaggggcgagggcagcagacctttcatgggcaaacctcagggctgctgtcgtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccataactggtaagtaccgcctatagactctataggcacacccctttggctcttatgcatgctgacagactaacagactgttcctttcctgggtcttttctgcaggcctgtacggaagtgttacttctgctctaaaagctgcggaattgtacccgcggccgatccaatcgatacagatctagcggccgcccgccaccatggctgaaaaagcggggtctacattttcacaccttctggttcctattcttctcctgattggctggattgtgggctgcatcataatgatttatgttgtcttctcttagaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 72 - DNA, 1922 bp, synthetic construct
  misc_feature 1..1922: note = Recombinant expression cassette or expression cassette component
  source 1..1922: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaaggtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagcggccgcccgccaccatggccgagaaggccggatctaccttcagccacctgctggtccctattctgctgctgatcggctggatcgtgggctgcatcatcatgatctacgtggtgttcagctgaaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 73 - DNA, 3871 bp, synthetic construct
  misc_feature 1..3871: note = Recombinant expression cassette or expression cassette component
  source 1..3871: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagccttcagattaaaaataactaaggtaagggccatgtgggtaggggaggtggtgtgagacggtcctgtctctcctctatctgcccatcggccctttggggaggaggaatgtgcccaaggactaaaaaaaggccctggagccagaggggcgagggcagcagacctttcatgggcaaacctcagggctgctgtcgtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccataactggtaagtaccgcctatagactctataggcacacccctttggctcttatgcatgctgacagactaacagactgttcctttcctgggtcttttctgcaggcctgtacggaagtgttacttctgctctaaaagctgcggaattgtacccgcggccgatccaatcgatacagatctagcggccgcccgccaccatggctgaaaaagcggggtctacattttcacaccttctggttcctattcttctcctgattggctggattgtgggctgcatcataatgatttatgttgtcttctcttagaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcagccagaagtcagatgctcaaggggcttcatgatgtccccataatttttggcagagggaaaaagatcggatcctcaggcgtagttcaccccgtcctcgaggccgcccgggtcgactaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccgcgcggccgtcagctgaacaccacgtagatcatgatgatgcagcccacgatccagccgatcagcagcagaatagggaccagcaggtggctgaaggtagatccggccttctcggccatggtggcggctagcctatagtgagtcgtattaagtactctagccttaagagctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttctgcagcttaagttcgagactgttgtgtcagaagcactgactgcgttagcaatttaactgtgataaactaccgcaataaagctctagagcttcggggatcgtcccacggagcggtgggtgccggcggctgtctgggaagggctccttggggggcagaggctttaaggtccccccggcgcccaccccggggcgggcagagccagcaggaatgtgcccggcgcccagagaggaatgcaacacttgtgagctgctattttggcagcagcggccccggccccctccgtgctcccccttcccccacaggagcccatataagcccaagctattgtgtggcctcagagttttgctattttaaacccgtcggacggagatacgtgagtgcccgaggggctgacacaagccagccagctgtcacctcccagggctggggacgctgataaggcagcgcttcggacccgaccctctgccgcagccccagatgctgtcatgtgaaagcccagactgcttttatccctgcttggacttctaatgctgtgcttttccttcagttcacaccagttaaaaatagaaaactgggcaccggcatctctgtctagagtgggcttggattgatatgctgagcaggactgctgattatcttcaggggccgggcagtgctgggtcctcccctcagagcttctcaaccatgtttgggatggtctagggatgaaactgctggacagctgagccatggagactggggagaggcctgcctggctgagaccaagccctgctgtaccatctgcatttgacagtgaggatgcccattgacaggaagcagacgtagagaaaaggagaatcagaatgatcgacaggcttcaaatcctacctctaacacttaactacgttccgcacgtttggtctcgggcaggccagttgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 74 - DNA, 3871 bp, synthetic construct
  misc_feature 1..3871: note = Recombinant expression cassette or expression cassette component
  source 1..3871: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagccttcagattaaaaataactaaggtaagggccatgtgggtaggggaggtggtgtgagacggtcctgtctctcctctatctgcccatcggccctttggggaggaggaatgtgcccaaggactaaaaaaaggccctggagccagaggggcgagggcagcagacctttcatgggcaaacctcagggctgctgtcgtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccataactggtaagtaccgcctatagactctataggcacacccctttggctcttatgcatgctgacagactaacagactgttcctttcctgggtcttttctgcaggcctgtacggaagtgttacttctgctctaaaagctgcggaattgtacccgcggccgatccaatcgatacagatctagcggccgcccgccaccatggctgaaaaagcggggtctacattttcacaccttctggttcctattcttctcctgattggctggattgtgggctgcatcataatgatttatgttgtcttctcttagaagctttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcagccagaagtcagatgctcaaggggcttcatgatgtccccataatttttggcagagggaaaaagatcggatcctcaggcgtagttcaccccgtcctcgaggccgcccgggtcgactaaaaaacctcccacacctccccctgaacctgaaacataaaatgaatgcaattgttgttgttaaactggcctgcccgagaccaaacgtgcggaacgtagttaagtgttagaggtaggatttgaagcctgtcgatcattctgattctccttttctctacgtctgcttcctgtcaatgggcatcctcactgtcaaatgcagatggtacagcagggcttggtctcagccaggcaggcctctccccagtctccatggctcagctgtccagcagtttcatccctagaccatcccaaacatggttgagaagctctgaggggaggacccagcactgcccggcccctgaagataatcagcagtcctgctcagcatatcaatccaagcccactctagacagagatgccggtgcccagttttctatttttaactggtgtgaactgaaggaaaagcacagcattagaagtccaagcagggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacgatccccgaagctctagagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccgccaccatggccgagaaggccggatctaccttcagccacctgctggtccctattctgctgctgatcggctggatcgtgggctgcatcatcatgatctacgtggtgttcagctgacggccgcgcggatccagacatgataagatacattgatgagtttggacaaaccacaactagaatgcagtgaaaaaaatgctttatttgtgaaatttgtgatgctattgctttatttgtaaccattataagctgcaataaacaagtgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 75 - DNA, 3862 bp, synthetic construct
  misc_feature 1..3862: note = Recombinant expression cassette or expression cassette component
  source 1..3862: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggcagatctcgaagacgcggaagaggccgcagagccggcagcaggccgcgggaaggaaggtccgctggattgagggccgaagggacgtagcagaaggacgtcccgcgcagaatccaggtggcaacacaggcgagcagccaaggaaaggacgatgatttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattggatccaaagcttctaagagaagacaacataaatcattatgatgcagcccacaatccagccaatcaggagaagaataggaaccagaaggtgtgaaaatgtagaccccgctttttcagccatggtggcgggcggccgctagatctgtatcgattggatcggccgcgggtacaattccgcagcttttagagcagaagtaacacttccgtacaggcctgcagaaaagacccaggaaaggaacagtctgttagtctgtcagcatgcataagagccaaaggggtgtgcctatagagtctataggcggtacttaccagttatggaggcgtctgctcagtctcagcggggactgggtgaggcagaggatggagagggctttaagcaggcatgtgggctggggcctggtgagccagccctgcggagggaggaatgtgcgacaggggacgggtggggcagggggatggcggtgggggtggggggtgttggctgctattttggcaggtgccagggacaaggctacaggaacatgtaccccacgccatataagcccatgtggtcctccagctgctcagataagctatttaaaaccagagcagatatgcagggaacagtcatgcaacataaaccagctgtccctcttgagaatcctgataaagcagaggccagcaacccaggcctgggagggccagctgggagcagggttggggggcagaaggcaacctccaagacactccataagtctcagcaccagaatcttggaaggcagagggcaagagttatgtgctgctccacttgaactgatgctgggggtaaagacatcttccaggctactggctcctaatggactgagcagccttaggcaggttgccggctctgccagccccagtgaggacatctgcaaggtgggtcttctccatgacgacagcagccctgaggtttgcccatgaaaggtctgctgccctcgcccctctggctccagggcctttttttagtccttgggcacattcctcctccccaaagggccgatgggcagatagaggagagacaggaccgtctcacaccacctcccctacccacatggcccttaccttagttatttttaatctgaaggctcgagttaagggcagccagaagtcagatgctcaaggggcttcatgatgtccccataatttttggcagagggaaaaagatcggatcctcaggcgtagttcaccccgtcctcgaggccgcccgggtcgactaaaaaacctcccacacctccccctgaacctgaaacataaaatgaatgcaattgttgttgttaaactggcctgcccgagaccaaacgtgcggaacgtagttaagtgttagaggtaggatttgaagcctgtcgatcattctgattctccttttctctacgtctgcttcctgtcaatgggcatcctcactgtcaaatgcagatggtacagcagggcttggtctcagccaggcaggcctctccccagtctccatggctcagctgtccagcagtttcatccctagaccatcccaaacatggttgagaagctctgaggggaggacccagcactgcccggcccctgaagataatcagcagtcctgctcagcatatcaatccaagcccactctagacagagatgccggtgcccagttttctatttttaactggtgtgaactgaaggaaaagcacagcattagaagtccaagcagggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacgatccccgaagctctagagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccgccaccatggccgagaaggccggatctaccttcagccacctgctggtccctattctgctgctgatcggctggatcgtgggctgcatcatcatgatctacgtggtgttcagctgacggccgcgcggatccagacatgataagatacattgatgagtttggacaaaccacaactagaatgcagtgaaaaaaatgctttatttgtgaaatttgtgatgctattgctttatttgtaaccattataagctgcaataaacaagtgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 76 - DNA, 105 bp, synthetic construct
  misc_feature 1..105: note = Recombinant expression cassette or expression cassette component
  source 1..105: mol_type = other DNA
atggccgagaaggaatctaccagcccccacctgatggtgcctattctgctgctggtgggctggatcgtcggctgcatcatcgtgatctacatcgtgttcttctga

SEQ ID NO: 77 - DNA, 108 bp, synthetic construct
  misc_feature 1..108: note = Recombinant expression cassette or expression cassette component
  source 1..108: mol_type = other DNA
atggccgagaaggccggatctaccttcagccacctgctggtccctattctgctgctgatcggctggatcgtgggctgcatcatcatgatctacgtggtgttcagctga

SEQ ID NO: 78 - DNA, 403 bp, synthetic construct
  misc_feature 1..403: note = Recombinant expression cassette or expression cassette component
  source 1..403: mol_type = other DNA
aactggcctgcccgagaccaaacgtgcggaacgtagttaagtgttagaggtaggatttgaagcctgtcgatcattctgattctccttttctctacgtctgcttcctgtcaatgggcatcctcactgtcaaatgcagatggtacagcagggcttggtctcagccaggcaggcctctccccagtctccatggctcagctgtccagcagtttcatccctagaccatcccaaacatggttgagaagctctgaggggaggacccagcactgcccggcccctgaagataatcagcagtcctgctcagcatatcaatccaagcccactctagacagagatgccggtgcccagttttctatttttaactggtgtgaactgaaggaaaagcacagcattagaagtccaagca

SEQ ID NO: 79 - DNA, 191 bp, synthetic construct
  misc_feature 1..191: note = Recombinant expression cassette or expression cassette component
  source 1..191: mol_type = other DNA
ccttcagattaaaaataactaaggtaagggccatgtgggtaggggaggtggtgtgagacggtcctgtctctcctctatctgcccatcggccctttggggaggaggaatgtgcccaaggactaaaaaaaggccctggagccagaggggcgagggcagcagacctttcatgggcaaacctcagggctgctgtc

SEQ ID NO: 80 - DNA, 96 bp, synthetic construct
  misc_feature 1..96: note = Recombinant expression cassette or expression cassette component
  source 1..96: mol_type = other DNA
gtaagtaccgcctatagactctataggcacacccctttggctcttatgcatgctgacagactaacagactgttcctttcctgggtcttttctgcag

SEQ ID NO: 81 - DNA, 133 bp, synthetic construct
  misc_feature 1..133: note = Recombinant expression cassette or expression cassette component
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 82-142 - skipped (61 entries)

SEQ ID NO: 143 - protein, 736 aa, unidentified
  source 1..736: note = Dependoparvovirus Adeno-associated virus serotype 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 144 - protein, 724 aa, unidentified; identical to SEQ ID NO: 41
  source 1..724: note = Dependoparvovirus Adeno-associated virus serotype 5

SEQ ID NO: 145 - protein, 736 aa, synthetic construct; identical to SEQ ID NO: 29
  REGION 1..736: note = Recombinant AAV Capsid Protein

SEQ ID NO: 146 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 147 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVSYQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 148 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSMVNQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 149 - protein, 736 aa, synthetic construct; identical to SEQ ID NO: 19
  REGION 1..736: note = Recombinant AAV Capsid Protein

SEQ ID NO: 150 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVGTQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 151 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVSYQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 152 - protein, 736 aa, synthetic construct; identical to SEQ ID NO: 30
  REGION 1..736: note = Recombinant AAV Capsid Protein

SEQ ID NO: 153 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNQDASLLWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 154 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNGMSFTWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 155 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 156 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNQDASLLWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 157 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNGMSFTWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 158 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 159 - protein, 726 aa, synthetic construct
  REGION 1..726: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTNNYNDPQFVDFAPDSTGEYRTTRPIGTRYLTRPL

SEQ ID NO: 160 - protein, 725 aa, synthetic construct
  REGION 1..725: note = Recombinant AAV Capsid Protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTSNYYKSNNVEFAVNTEGVYSEPRPIGTRYLTRNL

SEQ ID NO: 161 - protein, 725 aa, synthetic construct
  REGION 1..725: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEAAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 162 - protein, 727 aa, synthetic construct
  REGION 1..727: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 163 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVTVEMEWELKKENSKRWNPEIQYTNNYNDPQFVDFAPDSTGEYRTTRPIGTRYLTRPL

SEQ ID NO: 164 - protein, 726 aa, synthetic construct
  REGION 1..726: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSFAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 165 - protein, 726 aa, synthetic construct
  REGION 1..726: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 166 - protein, 726 aa, synthetic construct
  REGION 1..726: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTNNYNDPQFVDFAPDSTGEYRTTRPIGTRYLTRPL

SEQ ID NO: 167 - protein, 727 aa, synthetic construct
  REGION 1..727: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVTVEMEWELKKENSKRWNPEIQYTSNYYKSNNVEFAVNTEGVYSEPRPIGTRYLTRNL

SEQ ID NO: 168 - protein, 726 aa, synthetic construct
  REGION 1..726: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 169 - protein, 726 aa, synthetic construct
  REGION 1..726: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 170 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGNASGDWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSKMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVTVEMEWELKKENSKRWNPEIQYTNNYNDPQFVDFAPDSTGEYRTTRPIGTRYLTRNL

SEQ ID NO: 171 - protein, 735 aa, synthetic construct
  REGION 1..735: note = Recombinant AAV Capsid Protein
MSFVDHPPDWLEEVGEGLREFLGLEAGPPKPKPNQQHQDQARGLVLPGYNYLGPGNGLDRGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTNNYNDPQFVDFAPDSTGEYRTTRPIGTRYLTRPL

SEQ ID NO: 172 - protein, 735 aa, synthetic construct
  REGION 1..735: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 173 - protein, 726 aa, synthetic construct; identical to SEQ ID NO: 169
  REGION 1..726: note = Recombinant AAV Capsid Protein

SEQ ID NO: 174 - protein, 726 aa, synthetic construct
  REGION 1..726: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 175 - protein, 726 aa, synthetic construct
  REGION 1..726: note = Recombinant AAV Capsid Protein
MSFVDHPPDWLEEVGEGLREFLGLEAGPPKPKPNQQHQDQARGLVLPGYKYLGPGNGLDKGEPVNAADAAALEHDKAYDQQLKAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 176 - protein, 727 aa, synthetic construct
  REGION 1..727: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPFGLVEEGAKTAPTGKRIDDHFPKRKKARTEEDSKPSTSSDAEAGPSGSQQLQIPAQPASSLGADTMSAGGGGPLGDNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 177 - protein, 727 aa, synthetic construct
  REGION 1..727: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 178 - protein, 735 aa, synthetic construct
  REGION 1..735: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 179 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 180 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQEKLADDTSFGGNLGKAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 181 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 182 - protein, 735 aa, synthetic construct; identical to SEQ ID NO: 171
  REGION 1..735: note = Recombinant AAV Capsid Protein

SEQ ID NO: 183 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 184 - protein, 736 aa, synthetic construct; identical to SEQ ID NO: 18
  REGION 1..736: note = Recombinant AAV Capsid Protein

SEQ ID NO: 185 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVSYQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 186 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 187 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 188 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 189 - protein, 735 aa, synthetic construct
  REGION 1..735: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 190 - protein, 735 aa, synthetic construct
  REGION 1..735: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 191 - protein, 735 aa, synthetic construct
  REGION 1..735: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVSYQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 192 - protein, 735 aa, synthetic construct
  REGION 1..735: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 193 - protein, 735 aa, synthetic construct
  REGION 1..735: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 194 - protein, 735 aa, synthetic construct
  REGION 1..735: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 195 - protein, 727 aa, synthetic construct; identical to SEQ ID NO: 31
  REGION 1..727: note = Recombinant AAV Capsid Protein

SEQ ID NO: 196 - protein, 727 aa, synthetic construct; identical to SEQ ID NO: 176
  REGION 1..727: note = Recombinant AAV Capsid Protein

SEQ ID NO: 197 - protein, 727 aa, synthetic construct
  REGION 1..727: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPFGLVEEGAKTAPTGKRIDDHFPKRKKARTEEDSKPSTSSDAEAGPSGSQQLQIPAQPASSLGADTMSAGGGGPLGDNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVSYQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 198 - protein, 727 aa, synthetic construct
  REGION 1..727: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPFGLVEEGAKTAPTGKRIDDHFPKRKKARTEEDSKPSTSSDAEAGPSGSQQLQIPAQPASSLGADTMSAGGGGPLGDNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 199 - protein, 727 aa, synthetic construct
  REGION 1..727: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPFGLVEEGAKTAPTGKRIDDHFPKRKKARTEEDSKPSTSSDAEAGPSGSQQLQIPAQPASSLGADTMSAGGGGPLGDNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 200 - protein, 727 aa, synthetic construct
  REGION 1..727: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPFGLVEEGAKTAPTGKRIDDHFPKRKKARTEEDSKPSTSSDAEAGPSGSQQLQIPAQPASSLGADTMSAGGGGPLGDNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 201 - DNA, 2091 bp, Homo sapiens
  source 1..2091: mol_type = other DNA
atgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgcgcaccacgccgcccgagcccccaccctttgaggaccagcccgagcccgaggtctccgggtccgagtccgcgccctcgtccccggccaccgccccgctgcaggcccccacgctccgaggccccgagcctgcacgcgagacccccgccaagctggagcccaagcccatcatccccaaagccgagcccagggccaaggcccgcaagactgaggctcgagggctgaccaaggcgggggccaagaagaaggcgcggaaggaggccgcactggcggcagaggcggaggtggaggtggaagaggtccccaacaccatcctcatctgcatggtgatcctgctgaacatcggcctggccatcctctttgttcacctcctgacctga

SEQ ID NO: 202 - protein, 696 aa, Homo sapiens
MSGGRFDFDDGGAYCGGWEGGKAHGHGLCTGPKGQGEYSGSWNFGFEVAGVYTWPSGNTFEGYWSQGKRHGLGIETKGRWLYKGEWTHGFKGRYGIRQSSSSGAKYEGTWNNGLQDGYGTETYADGGTYQGQFTNGMRHGYGVRQSVPYGMAVVVRSPLRTSLSSLRSEHSNGTVAPDSPASPASDGPALPSPAIPRGGFALSLLANAEAAARAPKGGGLFQRGALLGKLRRAESRTSVGSQRSRVSFLKSDLSSGASDAASTASLGEAAEGADEAAPFEADIDATTTETYMGEWKNDKRSGFGVSERSSGLRYEGEWLDNLRHGYGCTTLPDGHREEGKYRHNVLVKDTKRRMLQLKSNKVRQKVEHSVEGAQRAAAIARQKAEIAASRTSHAKAKAEAAEQAALAANQESNIARTLARELAPDFYQPGPEYQKRRLLQEILENSESLLEPPDRGAGAAGLPQPPRESPQLHERETPRPEGGSPSPAGTPPQPKRPRPGVSKDGLLSPGAWNGEPSGEGSRSVTPSEGAGRRSPARPATERMAIEALQAPPAPSREPEVALYQGYHSYAVRTTPPEPPPFEDQPEPEVSGSESAPSSPATAPLQAPTLRGPEPARETPAKLEPKPIIPKAEPRAKARKTEARGLTKAGAKKKARKEAALAAEAEVEVEEVPNTILICMVILLNIGLAILFVHLLT

SEQ ID NO: 203 - DNA, 1728 bp, Homo sapiens
  source 1..1728: mol_type = other DNA
atgagcgccgccacccactcgcccatgatgcaggtggcgtccggcaacggtgaccgcgaccctttgccccccggatgggagatcaagatcgacccgcagaccggctggcccttcttcgtggaccacaacagccgcaccactacgtggaacgacccgcgcgtgccctctgagggccccaaggagactccatcctctgccaatggcccttcccgggagggctctaggctgccgcctgctagggaaggccaccctgtgtacccccagctccgaccaggctacattcccattcctgtgctccatgaaggcgctgagaaccggcaggtgcaccctttccatgtctatccccagcctgggatgcagcgattccgaactgaggcggcagcagcggctcctcagaggtcccagtcacctctgcggggcatgccagaaaccactcagccagataaacagtgtggacaggtggcagcggcggcggcagcccagcccccagcctcccacggacctgagcggtcccagtctccagctgcctctgactgctcatcctcatcctcctcggccagcctgccttcctccggcaggagcagcctgggcagtcaccagctcccgcgggggtacatctccattccggtgatacacgagcagaacgttacccggccagcagcccagccctccttccaccaagcccagaagacgcactacccagcgcagcagggggagtaccagacccaccagcctgtgtaccacaagatccagggggatgactgggagccccggcccctgcgggcggcatccccgttcaggtcatctgtccagggtgcatcgagccgggagggctcaccagccaggagcagcacgccactccactccccctcgcccatccgtgtgcacaccgtggtcgacaggcctcagcagcccatgacccatcgagaaactgcacctgtttcccagcctgaaaacaaaccagaaagtaagccaggcccagttggaccagaactccctcctggacacatcccaattcaagtgatccgcaaagaggtggattctaaacctgtttcccagaagcccccacctccctctgagaaggtagaggtgaaagttccccctgctccagttccttgtcctcctcccagccctggcccttctgctgtcccctcttcccccaagagtgtggctacagaagagagggcagcccccagcactgcccctgcagaagctacacctccaaaaccaggagaagccgaggctcccccaaaacatccaggagtgctgaaagtggaagccatcctggagaaggtacaggggctggagcaggctgtagacaactttgaaggcaagaagactgacaaaaagtacctgatgatcgaagagtatttgaccaaagagctgctggccctggattcagtggaccccgagggacgagccgatgtgcgtcaggccaggagagacggtgtcaggaaggttcagaccatcttggaaaaacttgaacagaaagccattgatgtcccaggtcaagtccaggtctatgaactccagcccagcaaccttgaagcagatcagccactgcaggcaatcatggagatgggtgccgtggcagcagacaagggcaagaaaaatgctggaaatgcagaagatccccacacagaaacccagcagccagaagccacagcagcagcgacttcaaaccccagcagcatgacagacacccctggtaacccagcagcaccgtag

SEQ ID NO: 204 - protein, 575 aa, Homo sapiens
MSAATHSPMMQVASGNGDRDPLPPGWEIKIDPQTGWPFFVDHNSRTTTWNDPRVPSEGPKETPSSANGPSREGSRLPPAREGHPVYPQLRPGYIPIPVLHEGAENRQVHPFHVYPQPGMQRFRTEAAAAAPQRSQSPLRGMPETTQPDKQCGQVAAAAAAQPPASHGPERSQSPAASDCSSSSSSASLPSSGRSSLGSHQLPRGYISIPVIHEQNVTRPAAQPSFHQAQKTHYPAQQGEYQTHQPVYHKIQGDDWEPRPLRAASPFRSSVQGASSREGSPARSSTPLHSPSPIRVHTVVDRPQQPMTHRETAPVSQPENKPESKPGPVGPELPPGHIPIQVIRKEVDSKPVSQKPPPPSEKVEVKVPPAPVPCPPPSPGPSAVPSSPKSVATEERAAPSTAPAEATPPKPGEAEAPPKHPGVLKVEAILEKVQGLEQAVDNFEGKKTDKKYLMIEEYLTKELLALDSVDPEGRADVRQARRDGVRKVQTILEKLEQKAIDVPGQVQVYELQPSNLEADQPLQAIMEMGAVAADKGKKNAGNAEDPHTETQQPEATAAATSNPSSMTDTPGNPAAP

SEQ ID NO: 205 - DNA, 528 bp, Homo sapiens
  source 1..528: mol_type = other DNA
atggacatcgccatccaccacccctggatccgccgccccttctttcctttccactcccccagccgcctctttgaccagttcttcggagagcacctgttggagtctgatcttttcccgacgtctacttccctgagtcccttctaccttcggccaccctccttcctgcgggcacccagctggtttgacactggactctcagagatgcgcctggagaaggacaggttctctgtcaacctggatgtgaagcacttctccccagaggaactcaaagttaaggtgttgggagatgtgattgaggtgcatggaaaacatgaagagcgccaggatgaacatggtttcatctccagggagttccacaggaaataccggatcccagctgatgtagaccctctcaccattacttcatccctgtcatctgatggggtcctcactgtgaatggaccaaggaaacaggtctctggccctgagcgcaccattcccatcacccgtgaagagaagcctgctgtcaccgcagcccccaagaaatag

SEQ ID NO: 206 - protein, 175 aa, Homo sapiens
MDIAIHHPWIRRPFFPFHSPSRLFDQFFGEHLLESDLFPTSTSLSPFYLRPPSFLRAPSWFDTGLSEMRLEKDRFSVNLDVKHFSPEELKVKVLGDVIEVHGKHEERQDEHGFISREFHRKYRIPADVDPLTITSSLSSDGVLTVNGPRKQVSGPERTIPITREEKPAVTAAPKK

SEQ ID NO: 207 - DNA, 1995 bp, Homo sapiens
  source 1..1995: mol_type = other DNA
atggagaccccgtcccagcggcgcgccacccgcagcggggcgcaggccagctccactccgctgtcgcccacccgcatcacccggctgcaggagaaggaggacctgcaggagctcaatgatcgcttggcggtctacatcgaccgtgtgcgctcgctggaaacggagaacgcagggctgcgccttcgcatcaccgagtctgaagaggtggtcagccgcgaggtgtccggcatcaaggccgcctacgaggccgagctcggggatgcccgcaagacccttgactcagtagccaaggagcgcgcccgcctgcagctggagctgagcaaagtgcgtgaggagtttaaggagctgaaagcgcgcaataccaagaaggagggtgacctgatagctgctcaggctcggctgaaggacctggaggctctgctgaactccaaggaggccgcactgagcactgctctcagtgagaagcgcacgctggagggcgagctgcatgatctgcggggccaggtggccaagcttgaggcagccctaggtgaggccaagaagcaacttcaggatgagatgctgcggcgggtggatgctgagaacaggctgcagaccatgaaggaggaactggacttccagaagaacatctacagtgaggagctgcgtgagaccaagcgccgtcatgagacccgactggtggagattgacaatgggaagcagcgtgagtttgagagccggctggcggatgcgctgcaggaactgcgggcccagcatgaggaccaggtggagcagtataagaaggagctggagaagacttattctgccaagctggacaatgccaggcagtctgctgagaggaacagcaacctggtgggggctgcccacgaggagctgcagcagtcgcgcatccgcatcgacagcctctctgcccagctcagccagctccagaagcagctggcagccaaggaggcgaagcttcgagacctggaggactcactggcccgtgagcgggacaccagccggcggctgctggcggaaaaggagcgggagatggccgagatgcgggcaaggatgcagcagcagctggacgagtaccaggagcttctggacatcaagctggccctggacatggagatccacgcctaccgcaagctcttggagggcgaggaggagaggctacgcctgtcccccagccctacctcgcagcgcagccgtggccgtgcttcctctcactcatcccagacacagggtgggggcagcgtcaccaaaaagcgcaaactggagtccactgagagccgcagcagcttctcacagcacgcacgcactagcgggcgcgtggccgtggaggaggtggatgaggagggcaagtttgtccggctgcgcaacaagtccaatgaggaccagtccatgggcaattggcagatcaagcgccagaatggagatgatcccttgctgacttaccggttcccaccaaagttcaccctgaaggctgggcaggtggtgacgatctgggctgcaggagctggggccacccacagcccccctaccgacctggtgtggaaggcacagaacacctggggctgcgggaacagcctgcgtacggctctcatcaactccactggggaagaagtggccatgcgcaagctggtgcgctcagtgactgtggttgaggacgacgaggatgaggatggagatgacctgctccatcaccaccacggctcccactgcagcagctcgggggaccccgctgagtacaacctgcgctcgcgcaccgtgctgtgcgggacctgcgggcagcctgccgacaaggcatctgccagcggctcaggagcccaggtgggcggacccatctcctctggctcttctgcctccagtgtcacggtcactcgcagctaccgcagtgtggggggcagtgggggtggcagcttcggggacaatctggtcacccgctcctacctcctgggcaactccagcccccgaacccagagcccccagaactgcagcatcatgtaa

SEQ ID NO: 208 - protein, 664 aa, Homo sapiens
METPSQRRATRSGAQASSTPLSPTRITRLQEKEDLQELNDRLAVYIDRVRSLETENAGLRLRITESEEVVSREVSGIKAAYEAELGDARKTLDSVAKERARLQLELSKVREEFKELKARNTKKEGDLIAAQARLKDLEALLNSKEAALSTALSEKRTLEGELHDLRGQVAKLEAALGEAKKQLQDEMLRRVDAENRLQTMKEELDFQKNIYSEELRETKRRHETRLVEIDNGKQREFESRLADALQELRAQHEDQVEQYKKELEKTYSAKLDNARQSAERNSNLVGAAHEELQQSRIRIDSLSAQLSQLQKQLAAKEAKLRDLEDSLARERDTSRRLLAEKEREMAEMRARMQQQLDEYQELLDIKLALDMEIHAYRKLLEGEEERLRLSPSPTSQRSRGRASSHSSQTQGGGSVTKKRKLESTESRSSFSQHARTSGRVAVEEVDEEGKFVRLRNKSNEDQSMGNWQIKRQNGDDPLLTYRFPPKFTLKAGQVVTIWAAGAGATHSPPTDLVWKAQNTWGCGNSLRTALINSTGEEVAMRKLVRSVTVVEDDEDEDGDDLLHHHHGSHCSSSGDPAEYNLRSRTVLCGTCGQPADKASASGSGAQVGGPISSGSSASSVTVTRSYRSVGGSGGGSFGDNLVTRSYLLGNSSPRTQSPQNCSIM

SEQ ID NO: 209 - DNA, 1719 bp, Homo sapiens
  source 1..1719: mol_type = other DNA
atggagaccccgtcccagcggcgcgccacccgcagcggggcgcaggccagctccactccgctgtcgcccacccgcatcacccggctgcaggagaaggaggacctgcaggagctcaatgatcgcttggcggtctacatcgaccgtgtgcgctcgctggaaacggagaacgcagggctgcgccttcgcatcaccgagtctgaagaggtggtcagccgcgaggtgtccggcatcaaggccgcctacgaggccgagctcggggatgcccgcaagacccttgactcagtagccaaggagcgcgcccgcctgcagctggagctgagcaaagtgcgtgaggagtttaaggagctgaaagcgcgcaataccaagaaggagggtgacctgatagctgctcaggctcggctgaaggacctggaggctctgctgaactccaaggaggccgcactgagcactgctctcagtgagaagcgcacgctggagggcgagctgcatgatctgcggggccaggtggccaagcttgaggcagccctaggtgaggccaagaagcaacttcaggatgagatgctgcggcgggtggatgctgagaacaggctgcagaccatgaaggaggaactggacttccagaagaacatctacagtgaggagctgcgtgagaccaagcgccgtcatgagacccgactggtggagattgacaatgggaagcagcgtgagtttgagagccggctggcggatgcgctgcaggaactgcgggcccagcatgaggaccaggtggagcagtataagaaggagctggagaagacttattctgccaagctggacaatgccaggcagtctgctgagaggaacagcaacctggtgggggctgcccacgaggagctgcagcagtcgcgcatccgcatcgacagcctctctgcccagctcagccagctccagaagcagctggcagccaaggaggcgaagcttcgagacctggaggactcactggcccgtgagcgggacaccagccggcggctgctggcggaaaaggagcgggagatggccgagatgcgggcaaggatgcagcagcagctggacgagtaccaggagcttctggacatcaagctggccctggacatggagatccacgcctaccgcaagctcttggagggcgaggaggagaggctacgcctgtcccccagccctacctcgcagcgcagccgtggccgtgcttcctctcactcatcccagacacagggtgggggcagcgtcaccaaaaagcgcaaactggagtccactgagagccgcagcagcttctcacagcacgcacgcactagcgggcgcgtggccgtggaggaggtggatgaggagggcaagtttgtccggctgcgcaacaagtccaatgaggaccagtccatgggcaattggcagatcaagcgccagaatggagatgatcccttgctgacttaccggttcccaccaaagttcaccctgaaggctgggcaggtggtgacgatctgggctgcaggagctggggccacccacagcccccctaccgacctggtgtggaaggcacagaacacctggggctgcgggaacagcctgcgtacggctctcatcaactccactggggaagaagtggccatgcgcaagctggtgcgctcagtgactgtggttgaggacgacgaggatgaggatggagatgacctgctccatcaccaccacgtgagtggtagccgccgctga

SEQ ID NO: 210 - protein, 572 aa, Homo sapiens
METPSQRRATRSGAQASSTPLSPTRITRLQEKEDLQELNDRLAVYIDRVRSLETENAGLRLRITESEEVVSREVSGIKAAYEAELGDARKTLDSVAKERARLQLELSKVREEFKELKARNTKKEGDLIAAQARLKDLEALLNSKEAALSTALSEKRTLEGELHDLRGQVAKLEAALGEAKKQLQDEMLRRVDAENRLQTMKEELDFQKNIYSEELRETKRRHETRLVEIDNGKQREFESRLADALQELRAQHEDQVEQYKKELEKTYSAKLDNARQSAERNSNLVGAAHEELQQSRIRIDSLSAQLSQLQKQLAAKEAKLRDLEDSLARERDTSRRLLAEKEREMAEMRARMQQQLDEYQELLDIKLALDMEIHAYRKLLEGEEERLRLSPSPTSQRSRGRASSHSSQTQGGGSVTKKRKLESTESRSSFSQHARTSGRVAVEEVDEEGKFVRLRNKSNEDQSMGNWQIKRQNGDDPLLTYRFPPKFTLKAGQVVTIWAAGAGATHSPPTDLVWKAQNTWGCGNSLRTALINSTGEEVAMRKLVRSVTVVEDDEDEDGDDLLHHHHVSGSRR

SEQ ID NO: 211 - DNA, 633 bp, Homo sapiens
  source 1..633: mol_type = other DNA
atggcggatgggagcagcgatgcggctagggaacctcgccctgcaccagccccaatcagacgccgctcctccaactaccgcgcttatgccacggagccgcacgccaagaaaaaatctaagatctccgcctcgagaaaattgcagctgaagactctgctgctgcagattgcaaagcaagagctggagcgagaggcggaggagcggcgcggagagaaggggcgcgctctgagcacccgctgccagccgctggagttggccgggctgggcttcgcggagctgcaggacttgtgccgacagctccacgcccgtgtggacaaggtggatgaagagagatacgacatagaggcaaaagtcaccaagaacatcacggagattgcagatctgactcagaagatctttgaccttcgaggcaagtttaagcggcccaccctgcggagagtgaggatctctgcagatgccatgatgcaggcgctgctgggggcccgggctaaggagtccctggacctgcgggcccacctcaagcaggtgaagaaggaggacaccgagaaggaaaaccgggaggtgggagactggcgcaagaacatcgatgcactgagtggaatggagggccgcaagaaaaagtttgagagctga

SEQ ID NO: 212 - protein, 210 aa, Homo sapiens
MADGSSDAAREPRPAPAPIRRRSSNYRAYATEPHAKKKSKISASRKLQLKTLLLQIAKQELEREAEERRGEKGRALSTRCQPLELAGLGFAELQDLCRQLHARVDKVDEERYDIEAKVTKNITEIADLTQKIFDLRGKFKRPTLRRVRISADAMMQALLGARAKESLDLRAHLKQVKKEDTEKENREVGDWRKNIDALSGMEGRKKKFES

SEQ ID NO: 213 - DNA, 1233 bp, Homo sapiens
  source 1..1233: mol_type = other DNA
atggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacagctcaagactgcagtgcagatgacgacaacttccttgtgcccatagcggtgggagctgccttggcaggagtacttattctagtgttgctggcttattttattggtctcaagcaccatcatgctggatatgagcaattttag

SEQ ID NO: 214 - protein, 410 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAQDCSADDDNFLVPIAVGAALAGVLILVLLAYFIGLKHHHAGYEQF

SEQ ID NO: 215 - DNA, 1233 bp, Homo sapiens
  source 1..1233: mol_type = other DNA
atggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacagcccaagagtgttcgctggatgatgacaccattctaatcccaattatagttggtgctggtctttcaggcttgattatcgttatagtgattgcttacgtaattggcagaagaaaaagttatgctggatatcagactctgtaa

SEQ ID NO: 216 - protein, 410 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAQECSLDDDTILIPIIVGAGLSGLIIVIVIAYVIGRRKSYAGYQTL

SEQ ID NO: 217 - DNA, 1236 bp, Homo sapiens
  source 1..1236: mol_type = other DNA
atggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacagctgaagaatgttctgctgactctgacctcaactttcttattcctgttgcagtgggtgtggccttgggcttccttataattgttgtctttatctcttatatgattggaagaaggaaaagtcgtactggttatcagtctgtgtaa

SEQ ID NO: 218 - protein, 411 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAEECSADSDLNFLIPVAVGVALGFLIIVVFISYMIGRRKSRTGYQSV

SEQ ID NO: 219 - DNA, 8616 bp, Homo sapiens
  source 1..8616: mol_type = other DNA
atgagctgcaacggaggctcccacccgcggatcaacactctgggccgcatgatccgcgccgagtctggcccggacctgcgctacgaggtgaccagcggcggcgggggcaccagcaggatgtactattctcggcgcggcgtgatcaccgaccagaactcggacggctactgtcaaaccggcacgatgtccaggcaccagaaccagaacaccatccaggagctgctgcagaactgctccgactgcttgatgcgagcagagctcatcgtgcagcctgaattgaagtatggagatggaatacaactgactcggagtcgagaattggatgagtgttttgcccaggccaatgaccaaatggaaatcctcgacagcttgatcagagagatgcggcagatgggccagccctgtgatgcttaccagaaaaggcttcttcagctccaagagcaaatgcgagccctttataaagccatcagtgtccctcgagtccgcagggccagctccaagggtggtggaggctacacttgtcagagtggctctggctgggatgagttcaccaaacatgtcaccagtgaatgtttggggtggatgaggcagcaaagggcggagatggacatggtggcctggggtgtggacctggcctcagtggagcagcacattaacagccaccggggcatccacaactccatcggcgactatcgctggcagctggacaaaatcaaagccgacctgcgcgagaaatctgcgatctaccagttggaggaggagtatgaaaacctgctgaaagcgtcctttgagaggatggatcacctgcgacagctgcagaacatcattcaggccacgtccagggagatcatgtggatcaatgactgcgaggaggaggagctgctgtacgactggagcgacaagaacaccaacatcgctcagaaacaggaggccttctccatacgcatgagtcaactggaagttaaagaaaaagagctcaataagctgaaacaagaaagtgaccaacttgtcctcaatcagcatccagcttcagacaaaattgaggcctatatggacactctgcagacgcagtggagttggattcttcagatcaccaagtgcattgatgttcatctgaaagaaaatgctgcctactttcagttttttgaagaggcgcagtctactgaagcatacctgaaggggctccaggactccatcaggaagaagtacccctgcgacaagaacatgcccctgcagcacctgctggaacagatcaaggagctggagaaagaacgagagaaaatccttgaatacaagcgtcaggtgcagaacttggtaaacaagtctaagaagattgtacagctgaagcctcgtaacccagactacagaagcaataaacccattattctcagagctctctgtgactacaaacaagatcagaaaatcgtgcataagggggatgagtgtatcctgaaggacaacaacgagcgcagcaagtggtacgtgacgggcccgggaggcgttgacatgcttgttccctctgtggggctgatcatccctcctccgaacccactggccgtggacctctcttgcaagattgagcagtactacgaagccatcttggctctgtggaaccagctctacatcaacatgaagagcctggtgtcctggcactactgcatgattgacatagagaagatcagggccatgacaatcgccaagctgaaaacaatgcggcaggaagattacatgaagacgatagccgaccttgagttacattaccaagagttcatcagaaatagccaaggctcagagatgtttggagatgatgacaagcggaaaatacagtctcagttcaccgatgcccagaagcattaccagaccctggtcattcagctccctggctatccccagcaccagacagtgaccacaactgaaatcactcatcatggaacctgccaagatgtcaaccataataaagtaattgaaaccaacagagaaaatgacaagcaagaaacatggatgctgatggagctgcagaagattcgcaggcagatagagcactgcgagggcaggatgactctcaaaaacctccctctagcagaccagggatcttctcaccacatcacagtgaaaattaacgagcttaagagtgtgcagaatgattcacaagcaattgctgaggttctcaaccagcttaaagatatgcttgccaacttcagaggttctgaaaagtactgctatttacagaatgaagtatttggactatttcagaaactggaaaatatcaatggtgttacagatggctacttaaatagcttatgcacagtaagggcactgctccaggctattctccaaacagaagacatgttaaaggtttatgaagccaggctcactgaggaggaaactgtctgcctggacctggataaagtggaagcttaccgctgtggactgaagaaaataaaaaatgacttgaacttgaagaagtcgttgttggccactatgaagacagaactacagaaagcccagcagatccactctcagacttcacagcagtatccactttatgatctggacttgggcaagttcggtgaaaaagtcacacagctgacagaccgctggcaaaggatagataaacagatcgactttaggttatgggacctggagaaacaaatcaagcaattgaggaattatcgtgataactatcaggctttctgcaagtggctctatgatgctaaacgccgccaggattccttagaatccatgaaatttggagattccaacacagtcatgcggtttttgaatgagcagaagaacttgcacagtgaaatatctggcaaacgagacaaatcagaggaagtacaaaaaattgctgaactttgcgccaattcaattaaggattatgagctccagctggcctcatacacctcaggactggaaactctgctgaacatacctatcaagaggaccatgattcagtccccttctggggtgattctgcaagaggctgcagatgttcatgctcggtacattgaactacttacaagatctggagactattacaggttcttaagtgagatgctgaagagtttggaagatctgaagctgaaaaataccaagatcgaagttttggaagaggagctcagactggcccgagatgccaactcggaaaactgtaataagaacaaattcctggatcagaacctgcagaaataccaggcagagtgttcccagttcaaagcgaagcttgcgagcctggaggagctgaagagacaggctgagctggatgggaagtcggctaagcaaaatctagacaagtgctacggccaaataaaagaactcaatgagaagatcacccgactgacttatgagattgaagatgaaaagagaagaagaaaatctgtggaagacagatttgaccaacagaagaatgactatgaccaactgcagaaagcaaggcaatgtgaaaaggagaaccttggttggcagaaattagagtctgagaaagccatcaaggagaaggagtacgagattgaaaggttgagggttctactgcaggaagaaggcacccggaagagagaatatgaaaatgagctggcaaaggtaagaaaccactataatgaggagatgagtaatttaaggaacaagtatgaaacagagattaacattacgaagaccaccatcaaggagatatccatgcaaaaagaggatgattccaaaaatcttagaaaccagcttgatagactttcaagggaaaatcgagatctgaaggatgaaattgtcaggctcaatgacagcatcttgcaggccactgagcagcgaaggcgagctgaagaaaacgcccttcagcaaaaggcctgtggctctgagataatgcagaagaagcagcatctggagatagaactgaagcaggtcatgcagcagcgctctgaggacaatgcccggcacaagcagtccctggaggaggctgccaagaccattcaggacaaaaataaggagatcgagagactcaaagctgagtttcaggaggaggccaagcgccgctgggaatatgaaaatgaactgagtaaggtaagaaacaattatgatgaggagatcattagcttaaaaaatcagtttgagaccgagatcaacatcaccaagaccaccatccaccagctcaccatgcagaaggaagaggataccagtggctaccgggctcagatagacaatctcacccgagaaaacaggagcttatctgaagaaataaagaggctgaagaacactctaacccagaccacagagaatctcaggagggtggaagaagacatccaacagcaaaaggccactggctctgaggtgtctcagaggaaacagcagctggaggttgagctgagacaagtcactcagatgcgaacagaggagagcgtaagatataagcaatctcttgatgatgctgccaaaaccatccaggataaaaacaaggagatagaaaggttaaaacaactgatcgacaaagaaacaaatgaccggaaatgcctggaagatgaaaacgcgagattacaaagggtccagtatgacctgcagaaagcaaacagtagtgcgacggagacaataaacaaactgaaggttcaggagcaagaactgacacgcctgaggatcgactatgaaagggtttcccaggagaggactgtgaaggaccaggatatcacgcggttccagaactctctgaaagagctgcagctgcagaagcagaaggtggaagaggagctgaatcggctgaagaggaccgcgtcagaagactcctgcaagaggaagaagctggaggaagagctggaaggcatgaggaggtcgctgaaggagcaagccatcaaaatcaccaacctgacccagcagctggagcaggcatccattgttaagaagaggagtgaggatgacctccggcagcagagggacgtgctggatggccacctgagggaaaagcagaggacccaggaagagctgaggaggctctcttctgaggtcgaggccctgaggcggcagttactccaggaacaggaaagtgtcaaacaagctcacttgaggaatgagcatttccagaaggcgatagaagataaaagcagaagcttaaatgaaagcaaaatagaaattgagaggctgcagtctctcacagagaacctgaccaaggagcacttgatgttagaagaagaactgcggaacctgaggctggagtacgatgacctgaggagaggacgaagcgaagcggacagtgataaaaatgcaaccatcttggaactaaggagccagctgcagatcagcaacaaccggaccctggaactgcaggggctgattaatgatttacagagagagagggaaaatttgagacaggaaattgagaaattccaaaagcaggctttagaggcatctaataggattcaggaatcaaagaatcagtgtactcaggtggtacaggaaagagagagccttctggtgaaaatcaaagtcctggagcaagacaaggcaaggctgcagaggctggaggatgagctgaatcgtgcaaaatcaactctagaggcagaaaccagggtgaaacagcgcctggagtgtgagaaacagcaaattcagaatgacctgaatcagtggaagactcaatattcccgcaaggaggaggctattaggaagatagaatcggaaagagaaaagagtgagagagagaagaacagtcttaggagtgagatcgaaagactccaagcagagatcaagagaattgaagagaggtgcaggcgtaagctggaggattctaccagggagacacagtcacagttagaaacagaacgctcccgatatcagagggagattgataaactcagacagcgcccatatgggtcccatcgagagacccagactgagtgtgagtggaccgttgacacctccaagctggtgtttgatgggctgaggaagaaggtgacagcaatgcagctctatgagtgtcagctgatcgacaaaacaaccttggacaaactattgaaggggaagaagtcagtggaagaagttgcttctgaaatccagccattccttcggggtgcaggatctatcgctggagcatctgcttctcctaaggaaaaatactctttggtagaggccaagagaaagaaattaatcagcccagaatccacagtcatgcttctggaggcccaggcagctacaggtggtataattgatccccatcggaatgagaagctgactgtcgacagtgccatagctcgggacctcattgacttcgatgaccgtcagcagatatatgcagcagaaaaagctatcactggttttgatgatccattttcaggcaagacagtatctgtttcagaagccatcaagaaaaatttgattgatagagaaaccggaatgcgcctgctggaagcccagattgcttcagggggtgtagtagaccctgtgaacagtgtctttttgccaaaagatgtcgccttggcccgggggctgattgatagagatttgtatcgatccctgaatgatccccgagatagtcagaaaaactttgtggatccagtcaccaaaaagaaggtcagttacgtgcagctgaaggaacggtgcagaatcgaaccacatactggtctgctcttgctttcagtacagaagagaagcatgtccttccaaggaatcagacaacctgtgaccgtcactgagctagtagattctggtatattgagaccgtccactgtcaatgaactggaatctggtcagatttcttatgacgaggttggtgagagaattaaggacttcctccagggttcaagctgcatagcaggcatatacaatgagaccacaaaacagaagcttggcatttatgaggccatgaaaattggcttagtccgacctggtactgctctggagttgctggaagcccaagcagctactggctttatagtggatcctgttagcaacttgaggttaccagtggaggaagcctacaagagaggtctggtgggcattgagttcaaagagaagctcctgtctgcagaacgagctgtcactgggtataatgatcctgaaacaggaaacatcatctctttgttccaagccatgaataaggaactcatcgaaaagggccacggtattcgcttattagaagcacagatcgcaaccggggggatcattgacccaaaggagagccatcgtttaccagttgacatagcatataagaggggctatttcaatgaggaactcagtgagattctctcagatccaagtgatgataccaaaggattttttgaccccaacactgaagaaaatcttacctatctgcaactaaaagaaagatgcattaaggatgaggaaacagggctctgtcttctgcctctgaaagaaaagaagaaacaggtgcagacatcacaaaagaataccctcaggaagcgtagagtggtcatagttgacccagaaaccaataaagaaatgtctgttcaggaggcctacaagaagggcctaattgattatgaaaccttcaaagaactgtgtgagcaggaatgtgaatgggaagaaataaccatcacgggatcagatggctccaccagggtggtcctggtagatagaaagacaggcagtcagtatgatattcaagatgctattgacaagggccttgttgacaggaagttctttgatcagtaccgatccggcagcctcagcctcactcaatttgctgacatgatctccttgaaaaatggtgtcggcaccagcagcagcatgggcagtggtgtcagcgatgatgtttttagcagctcccgacatgaatcagtaagtaagatttccaccatatccagcgtcaggaatttaaccataaggagcagctctttttcagacaccctggaagaatcgagccccattgcagccatctttgacacagaaaacctggagaaaatctccattacagaaggtatagagcggggcatcgttgacagcatcacgggtcagaggcttctggaggctcaggcctgcacaggtggcatcatccacccaaccacgggccagaagctgtcacttcaggacgcagtctcccagggtgtgattgaccaagacatggccaccaggctgaagcctgctcagaaagccttcataggcttcgagggtgtgaagggaaagaagaagatgtcagcagcagaggcagtgaaagaaaaatggctcccgtatgaggctggccagcgcttcctggagttccagtacctcacgggaggtcttgttgacccggaagtgcatgggaggataagcaccgaagaagccatccggaaggggttcatagatggccgcgccgcacagaggctgcaagacaccagcagctatgccaaaatcctgacctgccccaaaaccaaattaaaaatatcctataaggatgccataaatcgctccatggtagaagatatcactgggctgcgccttctggaagccgcctccgtgtcgtccaagggcttacccagcccttacaacatgtcttcggctccggggtcccgctccggctcccgctcgggatctcgctccggatctcgctccgggtcccgcagtgggtcccggagaggaagctttgacgccacagggaattcttcctactcttattcctactcatttagcagtagttctattgggcactag

SEQ ID NO: 220 - protein, 2871 aa, Homo sapiens
MSCNGGSHPRINTLGRMIRAESGPDLRYEVTSGGGGTSRMYYSRRGVITDQNSDGYCQTGTMSRHQNQNTIQELLQNCSDCLMRAELIVQPELKYGDGIQLTRSRELDECFAQANDQMEILDSLIREMRQMGQPCDAYQKRLLQLQEQMRALYKAISVPRVRRASSKGGGGYTCQSGSGWDEFTKHVTSECLGWMRQQRAEMDMVAWGVDLASVEQHINSHRGIHNSIGDYRWQLDKIKADLREKSAIYQLEEEYENLLKASFERMDHLRQLQNIIQATSREIMWINDCEEEELLYDWSDKNTNIAQKQEAFSIRMSQLEVKEKELNKLKQESDQLVLNQHPASDKIEAYMDTLQTQWSWILQITKCIDVHLKENAAYFQFFEEAQSTEAYLKGLQDSIRKKYPCDKNMPLQHLLEQIKELEKEREKILEYKRQVQNLVNKSKKIVQLKPRNPDYRSNKPIILRALCDYKQDQKIVHKGDECILKDNNERSKWYVTGPGGVDMLVPSVGLIIPPPNPLAVDLSCKIEQYYEAILALWNQLYINMKSLVSWHYCMIDIEKIRAMTIAKLKTMRQEDYMKTIADLELHYQEFIRNSQGSEMFGDDDKRKIQSQFTDAQKHYQTLVIQLPGYPQHQTVTTTEITHHGTCQDVNHNKVIETNRENDKQETWMLMELQKIRRQIEHCEGRMTLKNLPLADQGSSHHITVKINELKSVQNDSQAIAEVLNQLKDMLANFRGSEKYCYLQNEVFGLFQKLENINGVTDGYLNSLCTVRALLQAILQTEDMLKVYEARLTEEETVCLDLDKVEAYRCGLKKIKNDLNLKKSLLATMKTELQKAQQIHSQTSQQYPLYDLDLGKFGEKVTQLTDRWQRIDKQIDFRLWDLEKQIKQLRNYRDNYQAFCKWLYDAKRRQDSLESMKFGDSNTVMRFLNEQKNLHSEISGKRDKSEEVQKIAELCANSIKDYELQLASYTSGLETLLNIPIKRTMIQSPSGVILQEAADVHARYIELLTRSGDYYRFLSEMLKSLEDLKLKNTKIEVLEEELRLARDANSENCNKNKFLDQNLQKYQAECSQFKAKLASLEELKRQAELDGKSAKQNLDKCYGQIKELNEKITRLTYEIEDEKRRRKSVEDRFDQQKNDYDQLQKARQCEKENLGWQKLESEKAIKEKEYEIERLRVLLQEEGTRKREYENELAKVRNHYNEEMSNLRNKYETEINITKTTIKEISMQKEDDSKNLRNQLDRLSRENRDLKDEIVRLNDSILQATEQRRRAEENALQQKACGSEIMQKKQHLEIELKQVMQQRSEDNARHKQSLEEAAKTIQDKNKEIERLKAEFQEEAKRRWEYENELSKVRNNYDEEIISLKNQFETEINITKTTIHQLTMQKEEDTSGYRAQIDNLTRENRSLSEEIKRLKNTLTQTTENLRRVEEDIQQQKATGSEVSQRKQQLEVELRQVTQMRTEESVRYKQSLDDAAKTIQDKNKEIERLKQLIDKETNDRKCLEDENARLQRVQYDLQKANSSATETINKLKVQEQELTRLRIDYERVSQERTVKDQDITRFQNSLKELQLQKQKVEEELNRLKRTASEDSCKRKKLEEELEGMRRSLKEQAIKITNLTQQLEQASIVKKRSEDDLRQQRDVLDGHLREKQRTQEELRRLSSEVEALRRQLLQEQESVKQAHLRNEHFQKAIEDKSRSLNESKIEIERLQSLTENLTKEHLMLEEELRNLRLEYDDLRRGRSEADSDKNATILELRSQLQISNNRTLELQGLINDLQRERENLRQEIEKFQKQALEASNRIQESKNQCTQVVQERESLLVKIKVLEQDKARLQRLEDELNRAKSTLEAETRVKQRLECEKQQIQNDLNQWKTQYSRKEEAIRKIESEREKSEREKNSLRSEIERLQAEIKRIEERCRRKLEDSTRETQSQLETERSRYQREIDKLRQRPYGSHRETQTECEWTVDTSKLVFDGLRKKVTAMQLYECQLIDKTTLDKLLKGKKSVEEVASEIQPFLRGAGSIAGASASPKEKYSLVEAKRKKLISPESTVMLLEAQAATGGIIDPHRNEKLTVDSAIARDLIDFDDRQQIYAAEKAITGFDDPFSGKTVSVSEAIKKNLIDRETGMRLLEAQIASGGVVDPVNSVFLPKDVALARGLIDRDLYRSLNDPRDSQKNFVDPVTKKKVSYVQLKERCRIEPHTGLLLLSVQKRSMSFQGIRQPVTVTELVDSGILRPSTVNELESGQISYDEVGERIKDFLQGSSCIAGIYNETTKQKLGIYEAMKIGLVRPGTALELLEAQAATGFIVDPVSNLRLPVEEAYKRGLVGIEFKEKLLSAERAVTGYNDPETGNIISLFQAMNKELIEKGHGIRLLEAQIATGGIIDPKESHRLPVDIAYKRGYFNEELSEILSDPSDDTKGFFDPNTEENLTYLQLKERCIKDEETGLCLLPLKEKKKQVQTSQKNTLRKRRVVIVDPETNKEMSVQEAYKKGLIDYETFKELCEQECEWEEITITGSDGSTRVVLVDRKTGSQYDIQDAIDKGLVDRKFFDQYRSGSLSLTQFADMISLKNGVGTSSSMGSGVSDDVFSSSRHESVSKISTISSVRNLTIRSSSFSDTLEESSPIAAIFDTENLEKISITEGIERGIVDSITGQRLLEAQACTGGIIHPTTGQKLSLQDAVSQGVIDQDMATRLKPAQKAFIGFEGVKGKKKMSAAEAVKEKWLPYEAGQRFLEFQYLTGGLVDPEVHGRISTEEAIRKGFIDGRAAQRLQDTSSYAKILTCPKTKLKISYKDAINRSMVEDITGLRLLEAASVSSKGLPSPYNMSSAPGSRSGSRSGSRSGSRSGSRSGSRRGSFDATGNSSYSYSYSFSSSSIGH

SEQ ID NO: 221 - DNA, 6819 bp, Homo sapiens
  source 1..6819: mol_type = other DNA
atgagctgcaacggaggctcccacccgcggatcaacactctgggccgcatgatccgcgccgagtctggcccggacctgcgctacgaggtgaccagcggcggcgggggcaccagcaggatgtactattctcggcgcggcgtgatcaccgaccagaactcggacggctactgtcaaaccggcacgatgtccaggcaccagaaccagaacaccatccaggagctgctgcagaactgctccgactgcttgatgcgagcagagctcatcgtgcagcctgaattgaagtatggagatggaatacaactgactcggagtcgagaattggatgagtgttttgcccaggccaatgaccaaatggaaatcctcgacagcttgatcagagagatgcggcagatgggccagccctgtgatgcttaccagaaaaggcttcttcagctccaagagcaaatgcgagccctttataaagccatcagtgtccctcgagtccgcagggccagctccaagggtggtggaggctacacttgtcagagtggctctggctgggatgagttcaccaaacatgtcaccagtgaatgtttggggtggatgaggcagcaaagggcggagatggacatggtggcctggggtgtggacctggcctcagtggagcagcacattaacagccaccggggcatccacaactccatcggcgactatcgctggcagctggacaaaatcaaagccgacctgcgcgagaaatctgcgatctaccagttggaggaggagtatgaaaacctgctgaaagcgtcctttgagaggatggatcacctgcgacagctgcagaacatcattcaggccacgtccagggagatcatgtggatcaatgactgcgaggaggaggagctgctgtacgactggagcgacaagaacaccaacatcgctcagaaacaggaggccttctccatacgcatgagtcaactggaagttaaagaaaaagagctcaataagctgaaacaagaaagtgaccaacttgtcctcaatcagcatccagcttcagacaaaattgaggcctatatggacactctgcagacgcagtggagttggattcttcagatcaccaagtgcattgatgttcatctgaaagaaaatgctgcctactttcagttttttgaagaggcgcagtctactgaagcatacctgaaggggctccaggactccatcaggaagaagtacccctgcgacaagaacatgcccctgcagcacctgctggaacagatcaaggagctggagaaagaacgagagaaaatccttgaatacaagcgtcaggtgcagaacttggtaaacaagtctaagaagattgtacagctgaagcctcgtaacccagactacagaagcaataaacccattattctcagagctctctgtgactacaaacaagatcagaaaatcgtgcataagggggatgagtgtatcctgaaggacaacaacgagcgcagcaagtggtacgtgacgggcccgggaggcgttgacatgcttgttccctctgtggggctgatcatccctcctccgaacccactggccgtggacctctcttgcaagattgagcagtactacgaagccatcttggctctgtggaaccagctctacatcaacatgaagagcctggtgtcctggcactactgcatgattgacatagagaagatcagggccatgacaatcgccaagctgaaaacaatgcggcaggaagattacatgaagacgatagccgaccttgagttacattaccaagagttcatcagaaatagccaaggctcagagatgtttggagatgatgacaagcggaaaatacagtctcagttcaccgatgcccagaagcattaccagaccctggtcattcagctccctggctatccccagcaccagacagtgaccacaactgaaatcactcatcatggaacctgccaagatgtcaaccataataaagtaattgaaaccaacagagaaaatgacaagcaagaaacatggatgctgatggagctgcagaagattcgcaggcagatagagcactgcgagggcaggatgactctcaaaaacctccctctagcagaccagggatcttctcaccacatcacagtgaaaattaacgagcttaagagtgtgcagaatgattcacaagcaattgctgaggttctcaaccagcttaaagatatgcttgccaacttcagaggttctgaaaagtactgctatttacagaatgaagtatttggactatttcagaaactggaaaatatcaatggtgttacagatggctacttaaatagcttatgcacagtaagggcactgctccaggctattctccaaacagaagacatgttaaaggtttatgaagccaggctcactgaggaggaaactgtctgcctggacctggataaagtggaagcttaccgctgtggactgaagaaaataaaaaatgacttgaacttgaagaagtcgttgttggccactatgaagacagaactacagaaagcccagcagatccactctcagacttcacagcagtatccactttatgatctggacttgggcaagttcggtgaaaaagtcacacagctgacagaccgctggcaaaggatagataaacagatcgactttaggttatgggacctggagaaacaaatcaagcaattgaggaattatcgtgataactatcaggctttctgcaagtggctctatgatgctaaacgccgccaggattccttagaatccatgaaatttggagattccaacacagtcatgcggtttttgaatgagcagaagaacttgcacagtgaaatatctggcaaacgagacaaatcagaggaagtacaaaaaattgctgaactttgcgccaattcaattaaggattatgagctccagctggcctcatacacctcaggactggaaactctgctgaacatacctatcaagaggaccatgattcagtccccttctggggtgattctgcaagaggctgcagatgttcatgctcggtacattgaactacttacaagatctggagactattacaggttcttaagtgagatgctgaagagtttggaagatctgaagctgaaaaataccaagatcgaagttttggaagaggagctcagactggcccgagatgccaactcggaaaactgtaataagaacaaattcctggatcagaacctgcagaaataccaggcagagtgttcccagttcaaagcgaagcttgcgagcctggaggagctgaagagacaggctgagctggatgggaagtcggctaagcaaaatctagacaagtgctacggccaaataaaagaactcaatgagaagatcacccgactgacttatgagattgaagatgaaaagagaagaagaaaatctgtggaagacagatttgaccaacagaagaatgactatgaccaactgcagaaagcaaggcaatgtgaaaaggagaaccttggttggcagaaattagagtctgagaaagccatcaaggagaaggagtacgagattgaaaggttgagggttctactgcaggaagaaggcacccggaagagagaatatgaaaatgagctggcaaaggcatctaataggattcaggaatcaaagaatcagtgtactcaggtggtacaggaaagagagagccttctggtgaaaatcaaagtcctggagcaagacaaggcaaggctgcagaggctggaggatgagctgaatcgtgcaaaatcaactctagaggcagaaaccagggtgaaacagcgcctggagtgtgagaaacagcaaattcagaatgacctgaatcagtggaagactcaatattcccgcaaggaggaggctattaggaagatagaatcggaaagagaaaagagtgagagagagaagaacagtcttaggagtgagatcgaaagactccaagcagagatcaagagaattgaagagaggtgcaggcgtaagctggaggattctaccagggagacacagtcacagttagaaacagaacgctcccgatatcagagggagattgataaactcagacagcgcccatatgggtcccatcgagagacccagactgagtgtgagtggaccgttgacacctccaagctggtgtttgatgggctgaggaagaaggtgacagcaatgcagctctatgagtgtcagctgatcgacaaaacaaccttggacaaactattgaaggggaagaagtcagtggaagaagttgcttctgaaatccagccattccttcggggtgcaggatctatcgctggagcatctgcttctcctaaggaaaaatactctttggtagaggccaagagaaagaaattaatcagcccagaatccacagtcatgcttctggaggcccaggcagctacaggtggtataattgatccccatcggaatgagaagctgactgtcgacagtgccatagctcgggacctcattgacttcgatgaccgtcagcagatatatgcagcagaaaaagctatcactggttttgatgatccattttcaggcaagacagtatctgtttcagaagccatcaagaaaaatttgattgatagagaaaccggaatgcgcctgctggaagcccagattgcttcagggggtgtagtagaccctgtgaacagtgtctttttgccaaaagatgtcgccttggcccgggggctgattgatagagatttgtatcgatccctgaatgatccccgagatagtcagaaaaactttgtggatccagtcaccaaaaagaaggtcagttacgtgcagctgaaggaacggtgcagaatcgaaccacatactggtctgctcttgctttcagtacagaagagaagcatgtccttccaaggaatcagacaacctgtgaccgtcactgagctagtagattctggtatattgagaccgtccactgtcaatgaactggaatctggtcagatttcttatgacgaggttggtgagagaattaaggacttcctccagggttcaagctgcatagcaggcatatacaatgagaccacaaaacagaagcttggcatttatgaggccatgaaaattggcttagtccgacctggtactgctctggagttgctggaagcccaagcagctactggctttatagtggatcctgttagcaacttgaggttaccagtggaggaagcctacaagagaggtctggtgggcattgagttcaaagagaagctcctgtctgcagaacgagctgtcactgggtataatgatcctgaaacaggaaacatcatctctttgttccaagccatgaataaggaactcatcgaaaagggccacggtattcgcttattagaagcacagatcgcaaccggggggatcattgacccaaaggagagccatcgtttaccagttgacatagcatataagaggggctatttcaatgaggaactcagtgagattctctcagatccaagtgatgataccaaaggattttttgaccccaacactgaagaaaatcttacctatctgcaactaaaagaaagatgcattaaggatgaggaaacagggctctgtcttctgcctctgaaagaaaagaagaaacaggtgcagacatcacaaaagaataccctcaggaagcgtagagtggtcatagttgacccagaaaccaataaagaaatgtctgttcaggaggcctacaagaagggcctaattgattatgaaaccttcaaagaactgtgtgagcaggaatgtgaatgggaagaaataaccatcacgggatcagatggctccaccagggtggtcctggtagatagaaagacaggcagtcagtatgatattcaagatgctattgacaagggccttgttgacaggaagttctttgatcagtaccgatccggcagcctcagcctcactcaatttgctgacatgatctccttgaaaaatggtgtcggcaccagcagcagcatgggcagtggtgtcagcgatgatgtttttagcagctcccgacatgaatcagtaagtaagatttccaccatatccagcgtcaggaatttaaccataaggagcagctctttttcagacaccctggaagaatcgagccccattgcagccatctttgacacagaaaacctggagaaaatctccattacagaaggtatagagcggggcatcgttgacagcatcacgggtcagaggcttctggaggctcaggcctgcacaggtggcatcatccacccaaccacgggccagaagctgtcacttcaggacgcagtctcccagggtgtgattgaccaagacatggccaccaggctgaagcctgctcagaaagccttcataggcttcgagggtgtgaagggaaagaagaagatgtcagcagcagaggcagtgaaagaaaaatggctcccgtatgaggctggccagcgcttcctggagttccagtacctcacgggaggtcttgttgacccggaagtgcatgggaggataagcaccgaagaagccatccggaaggggttcatagatggccgcgccgcacagaggctgcaagacaccagcagctatgccaaaatcctgacctgccccaaaaccaaattaaaaatatcctataaggatgccataaatcgctccatggtagaagatatcactgggctgcgccttctggaagccgcctccgtgtcgtccaagggcttacccagcccttacaacatgtcttcggctccggggtcccgctccggctcccgctcgggatctcgctccggatctcgctccgggtcccgcagtgggtcccggagaggaagctttgacgccacagggaattcttcctactcttattcctactcatttagcagtagttctattgggcactag

SEQ ID NO: 222 - protein, 2272 aa, Homo sapiens
MSCNGGSHPRINTLGRMIRAESGPDLRYEVTSGGGGTSRMYYSRRGVITDQNSDGYCQTGTMSRHQNQNTIQELLQNCSDCLMRAELIVQPELKYGDGIQLTRSRELDECFAQANDQMEILDSLIREMRQMGQPCDAYQKRLLQLQEQMRALYKAISVPRVRRASSKGGGGYTCQSGSGWDEFTKHVTSECLGWMRQQRAEMDMVAWGVDLASVEQHINSHRGIHNSIGDYRWQLDKIKADLREKSAIYQLEEEYENLLKASFERMDHLRQLQNIIQATSREIMWINDCEEEELLYDWSDKNTNIAQKQEAFSIRMSQLEVKEKELNKLKQESDQLVLNQHPASDKIEAYMDTLQTQWSWILQITKCIDVHLKENAAYFQFFEEAQSTEAYLKGLQDSIRKKYPCDKNMPLQHLLEQIKELEKEREKILEYKRQVQNLVNKSKKIVQLKPRNPDYRSNKPIILRALCDYKQDQKIVHKGDECILKDNNERSKWYVTGPGGVDMLVPSVGLIIPPPNPLAVDLSCKIEQYYEAILALWNQLYINMKSLVSWHYCMIDIEKIRAMTIAKLKTMRQEDYMKTIADLELHYQEFIRNSQGSEMFGDDDKRKIQSQFTDAQKHYQTLVIQLPGYPQHQTVTTTEITHHGTCQDVNHNKVIETNRENDKQETWMLMELQKIRRQIEHCEGRMTLKNLPLADQGSSHHITVKINELKSVQNDSQAIAEVLNQLKDMLANFRGSEKYCYLQNEVFGLFQKLENINGVTDGYLNSLCTVRALLQAILQTEDMLKVYEARLTEEETVCLDLDKVEAYRCGLKKIKNDLNLKKSLLATMKTELQKAQQIHSQTSQQYPLYDLDLGKFGEKVTQLTDRWQRIDKQIDFRLWDLEKQIKQLRNYRDNYQAFCKWLYDAKRRQDSLESMKFGDSNTVMRFLNEQKNLHSEISGKRDKSEEVQKIAELCANSIKDYELQLASYTSGLETLLNIPIKRTMIQSPSGVILQEAADVHARYIELLTRSGDYYRFLSEMLKSLEDLKLKNTKIEVLEEELRLARDANSENCNKNKFLDQNLQKYQAECSQFKAKLASLEELKRQAELDGKSAKQNLDKCYGQIKELNEKITRLTYEIEDEKRRRKSVEDRFDQQKNDYDQLQKARQCEKENLGWQKLESEKAIKEKEYEIERLRVLLQEEGTRKREYENELAKASNRIQESKNQCTQVVQERESLLVKIKVLEQDKARLQRLEDELNRAKSTLEAETRVKQRLECEKQQIQNDLNQWKTQYSRKEEAIRKIESEREKSEREKNSLRSEIERLQAEIKRIEERCRRKLEDSTRETQSQLETERSRYQREIDKLRQRPYGSHRETQTECEWTVDTSKLVFDGLRKKVTAMQLYECQLIDKTTLDKLLKGKKSVEEVASEIQPFLRGAGSIAGASASPKEKYSLVEAKRKKLISPESTVMLLEAQAATGGIIDPHRNEKLTVDSAIARDLIDFDDRQQIYAAEKAITGFDDPFSGKTVSVSEAIKKNLIDRETGMRLLEAQIASGGVVDPVNSVFLPKDVALARGLIDRDLYRSLNDPRDSQKNFVDPVTKKKVSYVQLKERCRIEPHTGLLLLSVQKRSMSFQGIRQPVTVTELVDSGILRPSTVNELESGQISYDEVGERIKDFLQGSSCIAGIYNETTKQKLGIYEAMKIGLVRPGTALELLEAQAATGFIVDPVSNLRLPVEEAYKRGLVGIEFKEKLLSAERAVTGYNDPETGNIISLFQAMNKELIEKGHGIRLLEAQIATGGIIDPKESHRLPVDIAYKRGYFNEELSEILSDPSDDTKGFFDPNTEENLTYLQLKERCIKDEETGLCLLPLKEKKKQVQTSQKNTLRKRRVVIVDPETNKEMSVQEAYKKGLIDYETFKELCEQECEWEEITITGSDGSTRVVLVDRKTGSQYDIQDAIDKGLVDRKFFDQYRSGSLSLTQFADMISLKNGVGTSSSMGSGVSDDVFSSSRHESVSKISTISSVRNLTIRSSSFSDTLEESSPIAAIFDTENLEKISITEGIERGIVDSITGQRLLEAQACTGGIIHPTTGQKLSLQDAVSQGVIDQDMATRLKPAQKAFIGFEGVKGKKKMSAAEAVKEKWLPYEAGQRFLEFQYLTGGLVDPEVHGRISTEEAIRKGFIDGRAAQRLQDTSSYAKILTCPKTKLKISYKDAINRSMVEDITGLRLLEAASVSSKGLPSPYNMSSAPGSRSGSRSGSRSGSRSGSRSGSRRGSFDATGNSSYSYSYSFSSSSIGH

SEQ ID NO: 223 - DNA, 3357 bp, Homo sapiens
  source 1..3357: mol_type = other DNA
atggcgcggagcccgggacgcgcgtacgccctgctgcttctcctgatctgctttaacgttggaagtggacttcacttacaggtcttaagcacaagaaatgaaaataagctgcttcctaaacatcctcatttagtgcggcaaaagcgcgcctggatcaccgcccccgtggctcttcgggagggagaggatctgtccaagaagaatccaattgccaagatacattctgatcttgcagaagaaagaggactcaaaattacttacaaatacactggaaaagggattacagagccaccttttggtatatttgtctttaacaaagatactggagaactgaatgttaccagcattcttgatcgagaagaaacaccattttttctgctaacaggttacgctttggatgcaagaggaaacaatgtagagaaacccttagagctacgcattaaggttcttgatatcaatgacaacgaaccagtgttcacacaggatgtctttgttgggtctgttgaagagttgagtgcagcacatactcttgtgatgaaaatcaatgcaacagatgcagatgagcccaataccctgaattcgaaaatttcctatagaatcgtatctctggagcctgcttatcctccagtgttctacctaaataaagatacaggagagatttatacaaccagtgttaccttggacagagaggaacacagcagctacactttgacagtagaagcaagagatggcaatggagaagttacagacaaacctgtaaaacaagctcaagttcagattcgtattttggatgtcaatgacaatatacctgtagtagaaaataaagtgcttgaagggatggttgaagaaaatcaagtcaacgtagaagttacgcgcataaaagtgttcgatgcagatgaaataggttctgataattggctggcaaattttacatttgcatcaggaaatgaaggaggttatttccacatagaaacagatgctcaaactaacgaaggaattgtgacccttattaaggaagtagattatgaagaaatgaagaatcttgacttcagtgttattgtcgctaataaagcagcttttcacaagtcgattaggagtaaatacaagcctacacccattcccatcaaggtcaaagtgaaaaatgtgaaagaaggcattcattttaaaagcagcgtcatctcaatttatgttagcgagagcatggatagatcaagcaaaggccaaataattggaaattttcaagcttttgatgaggacactggactaccagcccatgcaagatatgtaaaattagaagatagagataattggatctctgtggattctgtcacatctgaaattaaacttgcaaaacttcctgattttgaatctagatatgttcaaaatggcacatacactgtaaagattgtggccatatcagaagattatcctagaaaaaccatcactggcacagtccttatcaatgttgaagacatcaacgacaactgtcccacactgatagagcctgtgcagacaatctgtcacgatgcagagtatgtgaatgttactgcagaggacctggatggacacccaaacagtggccctttcagtttctccgtcattgacaaaccacctggcatggcagaaaaatggaaaatagcacgccaagaaagtaccagtgtgctgctgcaacaaagtgagaaaaagcttgggagaagtgaaattcagttcctgatttcagacaatcagggttttagttgtcctgaaaagcaggtccttacactcacagtttgtgagtgtctgcatggcagcggctgcagggaagcacagcatgactcctatgtgggcctgggacccgcagcaattgcgctcatgattttggcctttctgctcctgctattggtaccacttttactgctgatgtgccattgcggaaagggcgccaaaggctttacccccatacctggcaccatagagatgctgcatccttggaataatgaaggagcaccacctgaagacaaggtggtgccatcatttctgccagtggatcaagggggcagtctagtaggaagaaatggagtaggaggtatggccaaggaagccacgatgaaaggaagtagctctgcttccattgtcaaagggcaacatgagatgtccgagatggatggaaggtgggaagaacacagaagcctgctttctggtagagctacccagtttacaggggccacaggcgctatcatgaccactgaaaccacgaagaccgcaagggccacaggggcttccagagacatggccggagctcaggcagctgctgttgcactgaacgaagaattcttaagaaattatttcactgataaagcggcctcttacactgaggaagatgaaaatcacacagccaaagattgccttctggtttattctcaggaagaaactgaatcgctgaatgcttctattggttgttgcagttttattgaaggagagctagatgaccgcttcttagatgatttgggacttaaattcaagacactagctgaagtttgcctgggtcaaaaaatagatataaataaggaaattgagcagagacaaaaacctgccacagaaacaagtatgaacacagcttcacattcactctgtgagcaaactatggttaattcagagaatacctactcctctggcagtagcttcccagttccaaaatctttgcaagaagccaatgcagagaaagtaactcaggaaatagtcactgaaagatctgtgtcttctaggcaggcgcaaaaggtagctacacctcttcctgacccaatggcttctagaaatgtgatagcaacagaaacttcctatgtcacagggtccactatgccaccaaccactgtgatcctgggtcctagccagccacagagccttattgtgacagagagggtgtatgctccagcttctaccttggtagatcagccttatgctaatgaaggtacagttgtggtcactgaaagagtaatacagcctcatgggggtggatcgaatcctctggaaggcactcagcatcttcaagatgtaccttacgtcatggtgagggaaagagagagcttccttgcccccagctcaggtgtgcagcctactctggccatgcctaatatagcagtaggacagaatgtgacagtgacagaaagagttctagcacctgcttccactctgcaatccagttaccagattcccactgaaaattctatgacggctaggaacaccacggtgtctggagctggagtccctggccctctgccagattttggtttagaggaatctggtcattctaattctaccataaccacatcttccaccagagttaccaagcatagcactgtacagcattcttactcctaa

SEQ ID NO: 224 - protein, 1118 aa, Homo sapiens
MARSPGRAYALLLLLICFNVGSGLHLQVLSTRNENKLLPKHPHLVRQKRAWITAPVALREGEDLSKKNPIAKIHSDLAEERGLKITYKYTGKGITEPPFGIFVFNKDTGELNVTSILDREETPFFLLTGYALDARGNNVEKPLELRIKVLDINDNEPVFTQDVFVGSVEELSAAHTLVMKINATDADEPNTLNSKISYRIVSLEPAYPPVFYLNKDTGEIYTTSVTLDREEHSSYTLTVEARDGNGEVTDKPVKQAQVQIRILDVNDNIPVVENKVLEGMVEENQVNVEVTRIKVFDADEIGSDNWLANFTFASGNEGGYFHIETDAQTNEGIVTLIKEVDYEEMKNLDFSVIVANKAAFHKSIRSKYKPTPIPIKVKVKNVKEGIHFKSSVISIYVSESMDRSSKGQIIGNFQAFDEDTGLPAHARYVKLEDRDNWISVDSVTSEIKLAKLPDFESRYVQNGTYTVKIVAISEDYPRKTITGTVLINVEDINDNCPTLIEPVQTICHDAEYVNVTAEDLDGHPNSGPFSFSVIDKPPGMAEKWKIARQESTSVLLQQSEKKLGRSEIQFLISDNQGFSCPEKQVLTLTVCECLHGSGCREAQHDSYVGLGPAAIALMILAFLLLLLVPLLLLMCHCGKGAKGFTPIPGTIEMLHPWNNEGAPPEDKVVPSFLPVDQGGSLVGRNGVGGMAKEATMKGSSSASIVKGQHEMSEMDGRWEEHRSLLSGRATQFTGATGAIMTTETTKTARATGASRDMAGAQAAAVALNEEFLRNYFTDKAASYTEEDENHTAKDCLLVYSQEETESLNASIGCCSFIEGELDDRFLDDLGLKFKTLAEVCLGQKIDINKEIEQRQKPATETSMNTASHSLCEQTMVNSENTYSSGSSFPVPKSLQEANAEKVTQEIVTERSVSSRQAQKVATPLPDPMASRNVIATETSYVTGSTMPPTTVILGPSQPQSLIVTERVYAPASTLVDQPYANEGTVVVTERVIQPHGGGSNPLEGTQHLQDVPYVMVRERESFLAPSSGVQPTLAMPNIAVGQNVTVTERVLAPASTLQSSYQIPTENSMTARNTTVSGAGVPGPLPDFGLEESGHSNSTITTSSTRVTKHSTVQHSYS

SEQ ID NO: 225 - DNA, 2238 bp, Homo sapiens
  source 1..2238: mol_type = other DNA
atggaggtgatgaacctgatggagcagcctatcaaggtgactgagtggcagcagacatacacctacgactcgggtatccactcgggcgccaacacctgcgtgccctccgtcagcagcaagggcatcatggaggaggatgaggcctgcgggcgccagtacacgctcaagaaaaccaccacttacacccagggggtgccccccagccaaggtgatctggagtaccagatgtccacaacagccagggccaaacgggtgcgggaggccatgtgccctggtgtgtcaggcgaggacagctcgcttctgctggccacccaggtggaggggcaggccaccaacctgcagcgactggccgagccgtcccagctgctcaagtcggccattgtgcatctcatcaactaccaggacgatgccgagctggccactcgcgccctgcccgagctcaccaaactgctcaacgacgaggacccggtggtggtgaccaaggcggccatgattgtgaaccagctgtcgaagaaggaggcgtcgcggcgggccctgatgggctcgccccagctggtggccgctgtcgtgcgtaccatgcagaataccagcgacctggacacagcccgctgcaccaccagcatcctgcacaacctctcccaccaccgggaggggctgctcgccatcttcaagtcgggtggcatccctgctctggtccgcatgctcagctcccctgtggagtcggtcctgttctatgccatcaccacgctgcacaacctgctcctgtaccaggagggcgccaagatggccgtgcgcctggccgacgggctgcaaaagatggtgcccctgctcaacaagaacaaccccaagttcctggccatcaccaccgactgcctgcagctcctggcctacggcaaccaggagagcaagctgatcatcctggccaatggtgggccccaggccctcgtgcagatcatgcgtaactacagttatgaaaagctgctctggaccaccagtcgtgtgctcaaggtgctatccgtgtgtcccagcaataagcctgccattgtggaggctggtgggatgcaggccctgggcaagcacctgaccagcaacagcccccgcctggtgcagaactgcctgtggaccctgcgcaacctctcagatgtggccaccaagcaggagggcctggagagtgtgctgaagattctggtgaatcagctgagtgtggatgacgtcaacgtcctcacctgtgccacgggcacactctccaacctgacatgcaacaacagcaagaacaagacgctggtgacacagaacagcggtgtggaggctctcatccatgccatcctgcgtgctggtgacaaggacgacatcacggagcctgccgtctgcgctctgcgccacctcactagccgccaccctgaggccgagatggcccagaactctgtgcgtctcaactatggcatcccagccatcgtgaagctgctcaaccagcccaaccagtggccactggtcaaggcaaccatcggcttgatcaggaatctggccctgtgcccagccaaccatgccccgctgcaggaggcagcggtcatcccccgcctcgtccaactgctggtgaaggcccaccaggatgcccagcgccacgtagctgcaggcacacagcagccctacacggatggtgtgaggatggaggagattgtggagggctgcaccggagcactgcacatcctcgcccgggaccccatgaaccgcatggagatcttccggctcaacaccattcccctgtttgtgcagctcctgtactcgtcggtggagaacatccagcgcgtggctgccggggtgctgtgtgagctggcccaggacaaggaggcggccgacgccattgatgcagagggggcctcggccccactcatggagttgctgcactcccgcaacgagggcactgccacctacgctgctgccgtcctgttccgcatctccgaggacaagaacccagactaccggaagcgcgtgtccgtggagctcaccaactccctcttcaagcatgacccggctgcctgggaggctgcccagagcatgattcccatcaatgagccctatggagatgacatggatgccacctaccgccccatgtactccagcgatgtgccccttgacccgctggagatgcacatggacatggatggagactaccccatcgacacctacagcgacggcctcaggcccccgtaccccactgcagaccacatgctggcctag

SEQ ID NO: 226 - protein, 745 aa, Homo sapiens
MEVMNLMEQPIKVTEWQQTYTYDSGIHSGANTCVPSVSSKGIMEEDEACGRQYTLKKTTTYTQGVPPSQGDLEYQMSTTARAKRVREAMCPGVSGEDSSLLLATQVEGQATNLQRLAEPSQLLKSAIVHLINYQDDAELATRALPELTKLLNDEDPVVVTKAAMIVNQLSKKEASRRALMGSPQLVAAVVRTMQNTSDLDTARCTTSILHNLSHHREGLLAIFKSGGIPALVRMLSSPVESVLFYAITTLHNLLLYQEGAKMAVRLADGLQKMVPLLNKNNPKFLAITTDCLQLLAYGNQESKLIILANGGPQALVQIMRNYSYEKLLWTTSRVLKVLSVCPSNKPAIVEAGGMQALGKHLTSNSPRLVQNCLWTLRNLSDVATKQEGLESVLKILVNQLSVDDVNVLTCATGTLSNLTCNNSKNKTLVTQNSGVEALIHAILRAGDKDDITEPAVCALRHLTSRHPEAEMAQNSVRLNYGIPAIVKLLNQPNQWPLVKATIGLIRNLALCPANHAPLQEAAVIPRLVQLLVKAHQDAQRHVAAGTQQPYTDGVRMEEIVEGCTGALHILARDPMNRMEIFRLNTIPLFVQLLYSSVENIQRVAAGVLCELAQDKEAADAIDAEGASAPLMELLHSRNEGTATYAAAVLFRISEDKNPDYRKRVSVELTNSLFKHDPAAWEAAQSMIPINEPYGDDMDATYRPMYSSDVPLDPLEMHMDMDGDYPIDTYSDGLRPPYPTADHMLA

SEQ ID NO: 227 - DNA, 1716 bp, Homo sapiens
  source 1..1716: mol_type = other DNA
atgagtgggggccgcttcgactttgatgatggaggggcgtactgcgggggctgggaggggggaaaggcccatgggcatggactgtgcacaggccccaagggccagggcgaatactctggctcctggaactttggctttgaggtggcaggtgtctacacctggcccagcggaaacacctttgagggatactggagccagggcaaacggcatgggctgggcatagagaccaaggggcgctggctctacaagggcgagtggacacatggcttcaagggacgctacggaatccggcagagctcaagcagcggtgccaagtatgagggcacctggaacaatggcctgcaagacggctatggcaccgagacctatgctgatggagggacgtaccaaggccagttcaccaacggcatgcgccatggctacggagtacgccagagcgtgccctacgggatggccgtggtggtgcgctcgccgctgcgcacgtcgctgtcgtccctgcgcagcgagcacagcaacggcacggtggccccggactctcccgcctcgccggcctccgacggccccgcgctgccctcgcccgccatcccgcgtggcggcttcgcgctcagcctcctggccaatgccgaggcggccgcgcgggcgcccaagggcggcggcctcttccagcggggcgcgctgctgggcaagctgcggcgcgcagagtcgcgcacgtccgtgggtagccagcgcagccgtgtcagcttccttaagagcgacctcagctcgggcgccagcgacgccgcgtccaccgccagcctgggagaggccgccgagggcgccgacgaggccgcacccttcgaggccgatatcgacgccaccaccaccgagacctacatgggcgagtggaagaacgacaaacgctcgggcttcggcgtgagcgaacgctccagtggcctccgctacgagggcgagtggctggacaacctgcgccacggctatggctgcaccacgctgcccgacggccaccgcgaggagggcaagtaccgccacaacgtgctggtcaaggacaccaagcgccgcatgctgcagctcaagagcaacaaggtccgccagaaagtggagcacagtgtggagggtgcccagcgcgccgctgctatcgcgcgccagaaggccgagattgccgcctccaggacaagccacgccaaggccaaagctgaggcagcggaacaggccgccctggctgccaaccaggagtccaacattgctcgcactttggccagggagctggctccggacttctaccagccaggtccggaatatcagaagcgccggctgctgcaggagatcctggagaactcggagagcctgctggagccccccgaccggggcgccggcgcagcgggcctcccacagccgccccgcgagagcccgcagctgcacgagcgtgagacccctcggcccgagggtggctccccgtcaccggccgggacgcccccgcagcccaagcggcccaggcccggggtgtccaaggacggcctgctgagcccaggcgcctggaacggcgagcccagcggtgagggcagccggtcagtcactccgtccgagggcgcgggccgccgcagccccgcgcgtccagccaccgagcgcatggccatcgaggctctgcaggcaccgcctgcgccgtcgcgggagccggaggtggcgctttaccagggctaccacagctatgctgtgcgc

SEQ ID NO: 228 - protein, 572 aa, Homo sapiens
MSGGRFDFDDGGAYCGGWEGGKAHGHGLCTGPKGQGEYSGSWNFGFEVAGVYTWPSGNTFEGYWSQGKRHGLGIETKGRWLYKGEWTHGFKGRYGIRQSSSSGAKYEGTWNNGLQDGYGTETYADGGTYQGQFTNGMRHGYGVRQSVPYGMAVVVRSPLRTSLSSLRSEHSNGTVAPDSPASPASDGPALPSPAIPRGGFALSLLANAEAAARAPKGGGLFQRGALLGKLRRAESRTSVGSQRSRVSFLKSDLSSGASDAASTASLGEAAEGADEAAPFEADIDATTTETYMGEWKNDKRSGFGVSERSSGLRYEGEWLDNLRHGYGCTTLPDGHREEGKYRHNVLVKDTKRRMLQLKSNKVRQKVEHSVEGAQRAAAIARQKAEIAASRTSHAKAKAEAAEQAALAANQESNIARTLARELAPDFYQPGPEYQKRRLLQEILENSESLLEPPDRGAGAAGLPQPPRESPQLHERETPRPEGGSPSPAGTPPQPKRPRPGVSKDGLLSPGAWNGEPSGEGSRSVTPSEGAGRRSPARPATERMAIEALQAPPAPSREPEVALYQGYHSYAVR

SEQ ID NO: 229 - DNA, 159 bp, Homo sapiens
  source 1..159: mol_type = other DNA
atggagaaagtccaatacctcactcgctcagctataagaagagcctcaaccattgaaatgcctcaacaagcacgtcaaaagctacagaatctatttatcaatttctgtctcatcttaatatgtctcttgctgatctgtatcatcgtgatgcttctctga

SEQ ID NO: 230 - protein, 52 aa, Homo sapiens
MEKVQYLTRSAIRRASTIEMPQQARQKLQNLFINFCLILICLLLICIIVMLL